(19) United States
(12) Patent Application Publication
(10) Pub. No.: US 2023/0338477 A1
(43) Pub. Date: Oct. 26, 2023

(54) ANTI-TFR:GAA AND ANTI-CD63:GAA INSERTION FOR TREATMENT OF POMPE DISEASE

(71) Applicant: Regeneron Pharmaceuticals, Inc., Tarrytown, NY (US)

(72) Inventors: Andrew Baik, Scarsdale, NY (US); Maria Praggastis, Cortlandt Manor, NY (US); Katherine Cygnar, New York, NY (US); Leah Sabin, Goldens Bridge, NY (US); Poulami Samai, New York, NY (US); Evangelos Pefanis, Somers, NY (US); Philip Calafati, Tarrytown, NY (US); Nicole Keating, Yorktown Heights, NY (US); Pascaline Aimé-Wilson, New York, NY (US); John Dugan, Hoboken, NJ (US); Min Gao, Woodcliff Lake, NJ (US); Robert Babb, River Edge, NJ (US); Anthony Forget, Cambridge, MA (US)

(21) Appl. No.: 18/163,698

(22) Filed: Feb. 2, 2023

Related U.S. Application Data
(60) Provisional application No. 63/369,902, filed on Jul. 29, 2022.
(60) Provisional application No. 63/306,040, filed on Feb. 2, 2022.

(51) Int. Cl. C07K 16/28; A61K 38/47; A61K 48/00; C12N 15/62; A61P 3/08; C12N 9/26

(52) U.S. Cl. CPC A61K 38/47; A61K 48/0066; A61P 3/08; C07K 16/2881; C07K 16/2896; C12N 9/2408; C12N 15/62; C12Y 302/0102; C07K 2317/622; C07K 2319/33; C12N 2310/20; C12N 2800/80

(57) ABSTRACT

Nucleic acid constructs and compositions that allow insertion of a multidomain therapeutic protein (e.g., GAA fusion protein) coding sequence into a target genomic locus such as an endogenous ALB locus and/or expression of the multidomain therapeutic protein (e.g., GAA fusion protein) coding sequence are also provided. The nucleic acid constructs and compositions can be used in methods of integration of a multidomain therapeutic protein (e.g., GAA fusion protein) nucleic acid into a target genomic locus, methods of expression of a multidomain therapeutic protein (e.g., GAA fusion protein) in a cell, methods of reducing glycogen accumulation, methods of treating Pompe disease or GAA deficiency in a subject, and method of preventing or reducing the onset of a sign or symptom of Pompe disease in a subject, including neonatal cells and subjects.

### CROSS-REFERENCE TO RELATED APPLICATIONS

This application claims the benefit of U.S. Application No. 63/306,040, filed Feb. 2, 2022, and U.S. Application No. 63/369,902, filed Jul. 29, 2022, each of which is herein incorporated by reference in its entirety for all purposes.

### REFERENCE TO A SEQUENCE LISTING SUBMITTED AS AN XML FILE VIA EFS WEB

The Sequence Listing written in file 590205SEQLIST.xml is 1.16 megabytes, was created on Feb. 2, 2023, and is hereby incorporated by reference.

### BACKGROUND

Gene therapy has long been recognized for its enormous potential in how human diseases are approached and treated. Instead of relying on drugs or surgery, patients with underlying genetic factors can be treated by directly targeting the underlying cause. Furthermore, by targeting the underlying genetic cause, gene therapy can provide the potential to effectively cure patients. However, clinical applications of gene therapy approaches still require improvement in several aspects. In addition, treatment early in life can present additional hurdles due to the unique environment in neonatal patients.

Pompe disease (PD), or glycogen storage disease type II, is a monogenic, lysosomal disease caused by a deficiency in the activity of the enzyme lysosomal acid alpha-glucosidase (GAA). GAA deficiency results in an accumulation of its substrate, glycogen, in the lysosomes of cells in tissues including skeletal and cardiac muscle. This aberrant accumulation of glycogen in myofibers results in progressive damage of muscle tissue, with symptoms that can include cardiomegaly, mild to profound muscle weakness, and ultimately death due to cardiac or respiratory failure. Infantile onset PD (IOPD) is associated with GAA activity of <1% of normal. It is severe and affects visceral organs, muscles, and the central nervous system (CNS). Late onset PD (LOPD) is associated with GAA activity of 2-40%. It is less severe, with primarily respiratory and skeletal muscle involvement.

The only approved therapy for PD is enzyme replacement therapy (ERT). Recombinant human (rh) GAA is delivered by intravenous infusion into patients every other week. While ERT has been very successful in treating the cardiac manifestations of PD, skeletal muscle and the CNS remain minimally treated by ERT. The primary mechanism by which rhGAA reaches lysosomes is through uptake by the cation-independent mannose 6-phosphate (M6P) receptor (CIMPR), which binds M6P on rhGAA. However, CI-MPR expression in skeletal muscle is very low, and rhGAA is poorly mannose 6-phosphorylated. In addition, CI-MPR may be misdirected into autophagosomes in affected cells, rather than lysosomes, while a large amount of the drug is also taken up by the liver, an organ that does not have primary pathology in PD. The ERT does not cross the blood-brain-barrier. In addition, PD can require treatment early in life, which presents additional hurdles due to the unique environment in neonatal and juvenile patients.

### SUMMARY

Nucleic acid constructs and compositions that allow insertion of a multidomain therapeutic protein (e.g., GAA fusion protein) coding sequence into a target genomic locus such as an endogenous ALB locus and/or expression of the multidomain therapeutic protein (e.g., GAA fusion protein) coding sequence are provided. The nucleic acid constructs and compositions can be used in methods of integrating or inserting a multidomain therapeutic protein (e.g., GAA fusion protein) nucleic acid into a target genomic locus in a cell or a population of cells or a subject, methods of expressing a multidomain therapeutic protein (e.g., GAA fusion protein) in a cell or a population of cells or a subject, methods of reducing glycogen accumulation in a cell or a population of cells or a subject, and methods of treating Pompe disease or GAA deficiency in a subject, and method of preventing or reducing the onset of a sign or symptom of Pompe disease in a subject such as subjects with reduced GAA activity or expression and in a subject diagnosed with Pompe disease, including neonatal subjects. In some embodiments the cell, population of cells, or subject is a neonatal cell, a neonatal population of cells, or a neonatal subject.

In one aspect, provided are methods of inserting a nucleic acid encoding a multidomain therapeutic protein comprising a delivery domain fused to a lysosomal alpha-glucosidase into a target genomic locus in a neonatal cell or a population of neonatal cells, optionally wherein the delivery domain is a CD63-binding delivery domain or a TfR-binding delivery domain. Some such methods comprise administering to the neonatal cell or the population of neonatal cells: (a) a nucleic acid construct comprising a coding sequence for the multidomain therapeutic protein comprising the delivery domain fused to the lysosomal alpha-glucosidase, optionally wherein the delivery domain is a CD63-binding delivery domain or a TfR-binding delivery domain; and (b) a nuclease agent or one or more nucleic acids encoding the nuclease agent, wherein the nuclease agent targets a nuclease target site in the target genomic locus, wherein the nuclease agent cleaves the nuclease target site, and the nucleic acid construct is inserted into the target genomic locus. In one aspect, provided are methods of inserting a nucleic acid encoding a multidomain therapeutic protein comprising a CD63-binding delivery domain fused to a lysosomal alpha-glucosidase into a target genomic locus in a neonatal cell or a population of neonatal cells. Some such methods comprise administering to the neonatal cell or the population of neonatal cells: (a) a nucleic acid construct comprising a coding sequence for the multidomain therapeutic protein comprising the CD63-binding delivery domain fused to the lysosomal alpha-glucosidase; and (b) a nuclease agent or one or more nucleic acids encoding the nuclease agent, wherein the nuclease agent targets a nuclease target site in the target genomic locus, wherein the nuclease agent cleaves the nuclease target site, and the nucleic acid construct is inserted into the target genomic locus. In another aspect, provided are methods of expressing a multidomain therapeutic protein comprising a delivery domain fused to a lysosomal alpha-glucosidase in a neonatal cell or a population of neonatal cells, optionally wherein the delivery domain is a CD63-binding delivery domain or a TfR-binding delivery domain. In another aspect, provided are methods of expressing a multidomain therapeutic protein comprising a delivery domain fused to a lysosomal alpha-glucosidase from a target genomic locus in a neonatal cell or a population of neonatal cells, optionally wherein the delivery domain is a CD63-binding delivery domain or a TfR-binding delivery domain. Some such methods comprise administering to the neonatal cell or the population of neonatal cells: (a) a nucleic acid construct comprising a coding sequence for the multidomain therapeutic protein comprising the delivery domain fused to the lysosomal alpha-glucosidase, optionally wherein the delivery domain is a CD63-binding delivery domain or a TfR-binding delivery domain; and (b) a nuclease agent or one or more nucleic acids encoding the nuclease agent, wherein the nuclease agent targets a nuclease target site in the target genomic locus, wherein the nuclease agent cleaves the nuclease target site, the nucleic acid construct is inserted into the target genomic locus to create a modified target genomic locus, and the multidomain therapeutic protein comprising the delivery domain fused to the lysosomal alpha-glucosidase is expressed from the modified target genomic locus. In another aspect, provided are methods of expressing a multidomain therapeutic protein comprising a CD63-binding delivery domain fused to a lysosomal alpha-glucosidase in a neonatal cell or a population of neonatal cells. In another aspect, provided are methods of expressing a multidomain therapeutic protein comprising a CD63-binding delivery domain fused to a lysosomal alpha-glucosidase from a target genomic locus in a neonatal cell or a population of neonatal cells. Some such methods comprise administering to the neonatal cell or the population of neonatal cells: (a) a nucleic acid construct comprising a coding sequence for the multidomain therapeutic protein comprising the CD63-binding delivery domain fused to the lysosomal alpha-glucosidase; and (b) a nuclease agent or one or more nucleic acids encoding the nuclease agent, wherein the nuclease agent targets a nuclease target site in the target genomic locus, wherein the nuclease agent cleaves the nuclease target site, the nucleic acid construct is inserted into the target genomic locus to create a modified target genomic locus, and the multidomain therapeutic protein comprising the CD63-binding delivery domain fused to the lysosomal alpha-glucosidase is expressed from the modified target genomic locus. In some such methods, the neonatal cell is a liver cell or the population of neonatal cells is a population of liver cells. In some such methods, the neonatal cell is a hepatocyte or the population of neonatal cells is a population of hepatocytes. In some such methods, the neonatal cell is a human cell or the population of neonatal cells is a population of human cells. In some such methods, the neonatal cell or the population of neonatal cells is from a human neonatal subject within 24 weeks after birth. In some such methods, the neonatal cell or the population of neonatal cells is from a human neonatal subject within 12 weeks after birth. In some such methods, the neonatal cell or the population of neonatal cells is from a human neonatal subject within 8 weeks after birth. In some such methods, the neonatal cell or the population of neonatal cells is from a human neonatal subject within 4 weeks after birth. In some such methods, the neonatal cell is in vitro or ex vivo or the population of neonatal cells is in vitro or ex vivo. In some such methods, the neonatal cell is in vivo in a neonatal subject or the population of neonatal cells is in vivo in a neonatal subject.

In another aspect, provided are methods of inserting a nucleic acid encoding a multidomain therapeutic protein comprising a delivery domain fused to a lysosomal alpha-glucosidase into a target genomic locus in a neonatal cell or a population of neonatal cells in a neonatal subject, optionally wherein the delivery domain is a CD63-binding delivery domain or a TfR-binding delivery domain. Some such methods comprise administering to the neonatal subject: (a) a nucleic acid construct comprising a coding sequence for the multidomain therapeutic protein comprising the delivery domain fused to the lysosomal alpha-glucosidase, optionally wherein the delivery domain is a CD63-binding delivery domain or a TfR-binding delivery domain; and (b) a nuclease agent or one or more nucleic acids encoding the nuclease agent, wherein the nuclease agent targets a nuclease target site in the target genomic locus, wherein the nuclease agent cleaves the nuclease target site, and the nucleic acid construct is inserted into the target genomic locus. In another aspect, provided are methods of inserting a nucleic acid encoding a multidomain therapeutic protein comprising a CD63-binding delivery domain fused to a lysosomal alpha-glucosidase into a target genomic locus in a neonatal cell or a population of neonatal cells in a neonatal subject. Some such methods comprise administering to the neonatal subject: (a) a nucleic acid construct comprising a coding sequence for the multidomain therapeutic protein comprising the CD63-binding delivery domain fused to the lysosomal alpha-glucosidase; and (b) a nuclease agent or one or more nucleic acids encoding the nuclease agent, wherein the nuclease agent targets a nuclease target site in the target genomic locus, wherein the nuclease agent cleaves the nuclease target site, and the nucleic acid construct is inserted into the target genomic locus. In another aspect, provided are methods of expressing a multidomain therapeutic protein comprising a delivery domain fused to a lysosomal alpha-glucosidase protein in a neonatal cell or a population of neonatal cells in a neonatal subject, optionally wherein the delivery domain is a CD63-binding delivery domain or a TfR-binding delivery domain. In another aspect, provided are methods of expressing a multidomain therapeutic protein comprising a delivery domain fused to a lysosomal alpha-glucosidase protein from a target genomic locus in a neonatal cell or a population of neonatal cells in a neonatal subject, optionally wherein the delivery domain is a CD63-binding delivery domain or a TfR-binding delivery domain. Some such methods comprise administering to the neonatal subject: (a) a nucleic acid construct comprising a coding sequence for the multidomain therapeutic protein comprising the delivery domain fused to the lysosomal alpha-glucosidase, optionally wherein the delivery domain is a CD63-binding delivery domain or a TfR-binding delivery domain; and (b) a nuclease agent or one or more nucleic acids encoding the nuclease agent, wherein the nuclease agent targets a nuclease target site in the target genomic locus, wherein the nuclease agent cleaves the nuclease target site, the nucleic acid construct is inserted into the target genomic locus to create a modified target genomic locus, and the multidomain therapeutic protein comprising the delivery domain fused to the lysosomal alpha-glucosidase is expressed from the modified target genomic locus. In another aspect, provided are methods of expressing a multidomain therapeutic protein comprising a CD63-binding delivery domain fused to a lysosomal alpha-glucosidase protein in a neonatal cell or a population of neonatal cells in a neonatal subject. In another aspect, provided are methods of expressing a multidomain therapeutic protein comprising a CD63-binding delivery domain fused to a lysosomal alpha-glucosidase protein from a target genomic locus in a neonatal cell or a population of neonatal cells in a neonatal subject. Some such methods comprise administering to the neonatal subject: (a) a nucleic acid construct comprising a coding sequence for the multidomain therapeutic protein comprising the CD63-binding delivery domain fused to the lysosomal alpha-glucosidase; and (b) a nuclease agent or one or more nucleic acids encoding the nuclease agent, wherein the nuclease agent targets a nuclease target site in the target genomic locus, wherein the nuclease agent cleaves the nuclease target site, the nucleic acid construct is inserted into the target genomic locus to create a modified target genomic locus, and the multidomain therapeutic protein comprising the CD63-binding delivery domain fused to the lysosomal alpha-glucosidase is expressed from the modified target genomic locus. In some such methods, the expressed multidomain therapeutic protein is delivered to and internalized by skeletal muscle and heart tissue in the subject. In some such methods, the neonatal cell is a liver cell or the population of neonatal cells is a population of liver cells. In some such methods, the neonatal cell is a hepatocyte or the population of neonatal cells is a population of hepatocytes. In some such methods, the neonatal cell is a human cell or the population of neonatal cells is a population of human cells. In some such methods, the subject has Pompe disease.

In another aspect, provided are methods of treating a lysosomal alpha-glucosidase deficiency in a neonatal subject in need thereof. Some such methods comprise administering to the neonatal subject: (a) a nucleic acid construct comprising a coding sequence for a multidomain therapeutic protein comprising a delivery domain fused to a lysosomal alpha-glucosidase, optionally wherein the delivery domain is a CD63-binding delivery domain or a TfR-binding delivery domain; and (b) a nuclease agent or one or more nucleic acids encoding the nuclease agent, wherein the nuclease agent targets a nuclease target site in a target genomic locus, wherein the nuclease agent cleaves the nuclease target site, the nucleic acid construct is inserted into the target genomic locus to create a modified target genomic locus, and the multidomain therapeutic protein comprising the delivery domain fused to the lysosomal alpha-glucosidase is expressed from the modified target genomic locus. Some such methods comprise administering to the neonatal subject: (a) a nucleic acid construct comprising a coding sequence for a multidomain therapeutic protein comprising a CD63-binding delivery domain fused to a lysosomal alpha-glucosidase; and (b) a nuclease agent or one or more nucleic acids encoding the nuclease agent, wherein the nuclease agent targets a nuclease target site in a target genomic locus, wherein the nuclease agent cleaves the nuclease target site, the nucleic acid construct is inserted into the target genomic locus to create a modified target genomic locus, and the multidomain therapeutic protein comprising the CD63-binding delivery domain fused to the lysosomal alpha-glucosidase is expressed from the modified target genomic locus. In another aspect, provided are methods of reducing glycogen accumulation in a tissue in a neonatal subject in need thereof. Some such methods comprise administering to the neonatal subject: (a) a nucleic acid construct comprising a coding sequence for a multidomain therapeutic protein comprising a delivery domain fused a lysosomal alpha-glucosidase, optionally wherein the delivery domain is a CD63-binding delivery domain or a TfR-binding delivery domain; and (b) a nuclease agent or one or more nucleic acids encoding the nuclease agent, wherein the nuclease agent targets a nuclease target site in a target genomic locus, wherein the nuclease agent cleaves the nuclease target site, the nucleic acid construct is inserted into the target genomic locus to create a modified target genomic locus, and the multidomain therapeutic protein comprising the delivery domain fused to the lysosomal alpha-glucosidase is expressed from the modified target genomic locus and reduces glycogen accumulation in the tissue. Some such methods comprise administering to the neonatal subject: (a) a nucleic acid construct comprising a coding sequence for a multidomain therapeutic protein comprising a CD63-binding delivery domain fused a lysosomal alpha-glucosidase; and (b) a nuclease agent or one or more nucleic acids encoding the nuclease agent, wherein the nuclease agent targets a nuclease target site in a target genomic locus, wherein the nuclease agent cleaves the nuclease target site, the nucleic acid construct is inserted into the target genomic locus to create a modified target genomic locus, and the multidomain therapeutic protein comprising the CD63-binding delivery domain fused to the lysosomal alpha-glucosidase is expressed from the modified target genomic locus and reduces glycogen accumulation in the tissue. In some such methods, the subject has Pompe disease. In another aspect, provided are methods of treating Pompe disease in a neonatal subject in need thereof. Some such methods comprise administering to the neonatal subject: (a) a nucleic acid construct comprising a coding sequence for a multidomain therapeutic protein comprising a delivery domain fused to a lysosomal alpha-glucosidase, optionally wherein the delivery domain is a CD63-binding delivery domain or a TfR-binding delivery domain; and (b) a nuclease agent or one or more nucleic acids encoding the nuclease agent, wherein the nuclease agent targets a nuclease target site in a target genomic locus, wherein the nuclease agent cleaves the nuclease target site, the nucleic acid construct is inserted into the target genomic locus to create a modified target genomic locus, and the multidomain therapeutic protein comprising the delivery domain fused to the lysosomal alpha-glucosidase is expressed from the modified target genomic locus, thereby treating the Pompe disease. Some such methods comprise administering to the neonatal subject: (a) a nucleic acid construct comprising a coding sequence for a multidomain therapeutic protein comprising a CD63-binding delivery domain fused to a lysosomal alpha-glucosidase; and (b) a nuclease agent or one or more nucleic acids encoding the nuclease agent, wherein the nuclease agent targets a nuclease target site in a target genomic locus, wherein the nuclease agent cleaves the nuclease target site, the nucleic acid construct is inserted into the target genomic locus to create a modified target genomic locus, and the multidomain therapeutic protein comprising the CD63-binding delivery domain fused to the lysosomal alpha-glucosidase is expressed from the modified target genomic locus, thereby treating the Pompe disease. In some such methods, the Pompe disease is infantile-onset Pompe disease. In some such methods, the method results in a therapeutically effective level of circulating multidomain therapeutic protein or lysosomal alpha-glucosidase in the subject. In some such methods, the method reduces glycogen accumulation in skeletal muscle, heart, or central nervous system tissue in the subject. In some such methods, the method reduces glycogen accumulation in skeletal muscle and heart tissue in the subject. In some such methods, the method results in reduced glycogen levels in skeletal muscle and/or heart tissue in the subject comparable to wild type levels at the same age. In some such methods, the method improves muscle strength in the subject or prevents loss of muscle strength in the subject compared to a control subject. In some such methods, the method results in the subject having muscle strength comparable to wild type levels at the same age.

In another aspect, provided are methods of preventing or reducing the onset of a sign or symptom of Pompe disease in a neonatal subject in need thereof. Some such methods comprise administering to the neonatal subject: (a) a nucleic acid construct comprising a coding sequence for a multidomain therapeutic protein comprising a delivery domain fused to a lysosomal alpha-glucosidase, optionally wherein the delivery domain is a CD63-binding delivery domain or a TfR-binding delivery domain; and (b) a nuclease agent or one or more nucleic acids encoding the nuclease agent, wherein the nuclease agent targets a nuclease target site in a target genomic locus, wherein the nuclease agent cleaves the nuclease target site, the nucleic acid construct is inserted into the target genomic locus to create a modified target genomic locus, and the multidomain therapeutic protein comprising the delivery domain fused to the lysosomal alpha-glucosidase is expressed from the modified target genomic locus, thereby preventing or reducing the onset of a sign or symptom of the Pompe disease in the subject. Some such methods comprise administering to the neonatal subject: (a) a nucleic acid construct comprising a coding sequence for a multidomain therapeutic protein comprising a CD63-binding delivery domain fused to a lysosomal alpha-glucosidase; and (b) a nuclease agent or one or more nucleic acids encoding the nuclease agent, wherein the nuclease agent targets a nuclease target site in a target genomic locus, wherein the nuclease agent cleaves the nuclease target site, the nucleic acid construct is inserted into the target genomic locus to create a modified target genomic locus, and the multidomain therapeutic protein comprising the CD63-binding delivery domain fused to the lysosomal alpha-glucosidase is expressed from the modified target genomic locus, thereby preventing or reducing the onset of a sign or symptom of the Pompe disease in the subject. In some such methods, the Pompe disease is infantile-onset Pompe disease. In some such methods, the Pompe disease is late-onset Pompe disease. In some such methods, the method results in a therapeutically effective level of circulating multidomain therapeutic protein or lysosomal alpha-glucosidase in the subject. In some such methods, the method prevents or reduces glycogen accumulation in skeletal muscle, heart, or central nervous system tissue in the subject. In some such methods, the method prevents or reduces glycogen accumulation in skeletal muscle and heart tissue in the subject.

In some such methods, the neonatal subject is a human subject within 24 weeks after birth. In some such methods, the neonatal subject is a human subject within 12 weeks after birth. In some such methods, the neonatal subject is a human subject within 8 weeks after birth. In some such methods, the neonatal subject is a human subject within 4 weeks after birth.

In some such methods, the method results in increased expression of the multidomain therapeutic protein in the subject compared to a method comprising administering an episomal expression vector encoding the multidomain therapeutic protein to a control subject. In some such methods, the method results in increased serum levels of the multidomain therapeutic protein in the subject compared to a method comprising administering an episomal expression vector encoding the multidomain therapeutic protein to a control subject. In some such methods, the method results in serum levels of the multidomain therapeutic protein in the subject of at least about 1 µg/mL, at least about 2 µg/mL, at least about 3 µg/mL, at least about 4 µg/mL, at least about 5 µg/mL, at least about 6 µg/mL, at least about 7 µg/mL, at least about 8 µg/mL, at least about 9 µg/mL, or at least about 10 µg/mL. In some such methods, the method results in serum levels of the multidomain therapeutic protein in the subject of at least about 2 µg/mL or at least about 5 µg/mL. In some such methods, the method results in serum levels of the multidomain therapeutic protein in the subject of between about 2 µg/mL and about 30 µg/mL or between about 2 µg/mL and about 20 µg/mL. In some such methods, the method results in serum levels of the multidomain therapeutic protein in the subject of between about 5 µg/mL and about 30 µg/mL or between about 5 µg/mL and about 20 µg/mL. In some such methods, the method achieves lysosomal alpha-glucosidase activity levels of at least about 40% of normal, at least about 45% of normal, at least about 50%, at least about 60%, at least about 70%, at least about 80%, at least about 90%, or 100% of normal. In some such methods, the subject has infantile-onset Pompe disease, and the method achieves lysosomal alpha-glucosidase activity levels of at least about 1% or more than about 1% of normal; or . In some such methods, the subject has late-onset Pompe disease, and the method achieves lysosomal alpha-glucosidase activity levels of at least about 40% of normal, or more than about 40% of normal. In some such methods, the method increases lysosomal alpha-glucosidase activity over the subject’s baseline lysosomal alpha-glucosidase activity by at least about 1%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or at least about 100%. In some such methods, the expression or activity of the multidomain therapeutic protein is at least 50% of the expression or activity of the multidomain therapeutic protein at a peak level of expression measured for the subject at six months after the administering. In some such methods, the expression or activity of the multidomain therapeutic protein is at least 50% of the expression or activity of the multidomain therapeutic protein at a peak level of expression measured for the subject at one year after the administering. In some such methods, the expression or activity of the multidomain therapeutic protein is at least 60% of the expression or activity of the multidomain therapeutic protein at a peak level of expression measured for the subject at six months after the administering. In some such methods, the expression or activity of the multidomain therapeutic protein is at least 50% of the expression or activity of the multidomain therapeutic protein at a peak level of expression measured for the subject at two years after the administering. In some such methods, the expression or activity of the multidomain therapeutic protein is at least 60% of the expression or activity of the multidomain therapeutic protein at a peak level of expression measured for the subject at two years after the administering. In some such methods, the expression or activity of the multidomain therapeutic protein is at least 60% of the expression or activity of the multidomain therapeutic protein at a peak level of expression measured for the subject at six months after the administering.

In some such methods, the subject is a human subject.

In some such methods, the method further comprises assessing preexisting AAV immunity in the neonatal subject prior to administering the nucleic acid construct to the subject. In some such methods, the preexisting AAV immunity is preexisting AAV8 immunity. In some such methods, assessing preexisting AAV immunity comprises assessing immunogenicity using a total antibody immune assay or a neutralizing antibody assay.

In some such methods, the nucleic acid construct is administered simultaneously with the nuclease agent or the one or more nucleic acids encoding the nuclease agent. In some such methods, the nucleic acid construct is not administered simultaneously with the nuclease agent or the one or more nucleic acids encoding the nuclease agent. In some such methods, the nucleic acid construct is administered prior to the nuclease agent or the one or more nucleic acids encoding the nuclease agent. In some such methods, the nucleic acid construct is administered after the nuclease agent or the one or more nucleic acids encoding the nuclease agent.

In some such methods, the CD63-binding delivery domain is fused to the lysosomal alpha-glucosidase protein via a peptide linker. In some such methods, the coding sequence for the CD63-binding delivery domain is codon-optimized or CpG-depleted. In some such methods, the coding sequence for the CD63-binding delivery domain is codon-optimized and CpG-depleted. In some such methods, the CD63-binding delivery domain comprises an anti-CD63 antigen-binding protein. In some such methods, the CD63-binding delivery domain comprises an anti-CD63 antibody, antibody fragment, or single-chain variable fragment (scFv). In some such methods, the CD63-binding delivery domain is the single-chain variable fragment (scFv). In some such methods, the scFv comprises the sequence set forth in SEQ ID NO: 183. In some such methods, the scFv consists of the sequence set forth in SEQ ID NO: 183. In some such methods, the scFv coding sequence is at least 90%, at least 91%, at least 92%, at least 93%, at least 94%, at least 95%, at least 96%, at least 97%, at least 98%, or at least 99% identical to any one of SEQ ID NOS: 184-192, optionally wherein the scFv coding sequence is at least 90%, at least 91%, at least 92%, at least 93%, at least 94%, at least 95%, at least 96%, at least 97%, at least 98%, or at least 99% identical to SEQ ID NO: 186. In some such methods, the scFv coding sequence is at least 90%, at least 91%, at least 92%, at least 93%, at least 94%, at least 95%, at least 96%, at least 97%, at least 98%, or at least 99% identical to any one of SEQ ID NOS: 184-192 and encodes an scFv comprising SEQ ID NO: 183, optionally wherein the scFv coding sequence is at least 90%, at least 91%, at least 92%, at least 93%, at least 94%, at least 95%, at least 96%, at least 97%, at least 98%, or at least 99% identical to SEQ ID NO: 186 and encodes an scFv comprising SEQ ID NO: 183. In some such methods, the scFv coding sequence is at least 90%, at least 91%, at least 92%, at least 93%, at least 94%, at least 95%, at least 96%, at least 97%, at least 98%, or at least 99% identical to any one of SEQ ID NOS: 184-192, is codon-optimized and CpG-depleted, and encodes an scFv comprising SEQ ID NO: 183, optionally wherein the scFv coding sequence is at least 90%, at least 91%, at least 92%, at least 93%, at least 94%, at least 95%, at least 96%, at least 97%, at least 98%, or at least 99% identical to SEQ ID NO: 186, is codon-optimized and CpG-depleted, and encodes an scFv comprising SEQ ID NO: 183. In some such methods, the scFv coding sequence comprises the sequence set forth in any one of SEQ ID NOS: 184-192, optionally wherein the scFv coding sequence comprises the sequence set forth in SEQ ID NO: 186. In some such methods, the scFv coding sequence consists of the sequence set forth in any one of SEQ ID NOS: 184-192, optionally wherein the scFv coding sequence consists of the sequence set forth in SEQ ID NO: 186.

In some such methods, the lysosomal alpha-glucosidase lacks the lysosomal alpha-glucosidase signal peptide and propeptide. In some such methods, the lysosomal alpha-glucosidase comprises the sequence set forth in SEQ ID NO: 173. In some such methods, the lysosomal alpha-glucosidase consists of the sequence set forth in SEQ ID NO: 173. In some such methods, the lysosomal alpha-glucosidase coding sequence is codon-optimized or CpG-depleted. In some such methods, the lysosomal alpha-glucosidase coding sequence is codon-optimized and CpG-depleted. In some such methods, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and 205-212, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176. In some such methods, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and 205-212 and encodes a lysosomal alpha-glucosidase protein comprising SEQ ID NO: 173, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176 and encodes a lysosomal alpha-glucosidase protein comprising SEQ ID NO: 173. In some such methods, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and 205-212, is codon-optimized and CpG-depleted, and encodes a lysosomal alpha-glucosidase protein comprising SEQ ID NO: 173, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176, is codon-optimized and CpG-depleted, and encodes a lysosomal alpha-glucosidase protein comprising SEQ ID NO: 173. In some such methods, the lysosomal alpha-glucosidase coding sequence comprises the sequence set forth in any one of SEQ ID NOS: 174-182 and 205-212, optionally wherein the lysosomal alpha-glucosidase coding sequence comprises the sequence set forth in SEQ ID NO: 176. In some such methods, the lysosomal alpha-glucosidase coding sequence consists of the sequence set forth in any one of SEQ ID NOS: 174-182 and 205-212, optionally wherein the lysosomal alpha-glucosidase coding sequence consists of the sequence set forth in SEQ ID NO: 176.

In some such methods, the lysosomal alpha-glucosidase lacks the lysosomal alpha-glucosidase signal peptide and propeptide. In some such methods, the lysosomal alpha-glucosidase comprises the sequence set forth in SEQ ID NO: 173. In some such methods, the lysosomal alpha-glucosidase consists of the sequence set forth in SEQ ID NO: 173. In some such methods, the lysosomal alpha-glucosidase coding sequence is codon-optimized or CpG-depleted. In some such methods, the lysosomal alpha-glucosidase coding sequence is codon-optimized and CpG-depleted. In some such methods, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176. In some such methods, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and encodes a lysosomal alpha-glucosidase protein comprising SEQ ID NO: 173, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176 and encodes a lysosomal alpha-glucosidase protein comprising SEQ ID NO: 173. In some such methods, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182, is codon-optimized and CpG-depleted, and encodes a lysosomal alpha-glucosidase protein comprising SEQ ID NO: 173, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176, is codon-optimized and CpG-depleted, and encodes a lysosomal alpha-glucosidase protein comprising SEQ ID NO: 173. In some such methods, the lysosomal alpha-glucosidase coding sequence comprises the sequence set forth in any one of SEQ ID NOS: 174-182, optionally wherein the lysosomal alpha-glucosidase coding sequence comprises the sequence set forth in SEQ ID NO: 176. In some such methods, the lysosomal alpha-glucosidase coding sequence consists of the sequence set forth in any one of SEQ ID NOS: 174-182, optionally wherein the lysosomal alpha-glucosidase coding sequence consists of the sequence set forth in SEQ ID NO: 176.

In some such methods, the coding sequence for the multidomain therapeutic protein is codon-optimized or CpG-depleted. In some such methods, the coding sequence for the multidomain therapeutic protein is codon-optimized and CpG-depleted. In some such methods, the multidomain therapeutic protein comprises the sequence set forth in SEQ ID NO: 193. In some such methods, the multidomain therapeutic protein consists of the sequence set forth in SEQ ID NO: 193. In some such methods, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 194-202, optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 196, optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 736. In some such methods, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 194-202, and the multidomain therapeutic protein comprises the sequence set forth in SEQ ID NO: 193, optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 196, and the multidomain therapeutic protein comprises the sequence set forth in SEQ ID NO: 193, optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 736, and the multidomain therapeutic protein comprises the sequence set forth in SEQ ID NO: 193. In some such methods, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 194-202 and is codon-optimized and CpG-depleted, and the multidomain therapeutic protein comprises the sequence set forth in SEQ ID NO: 193, optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 196 and is codon-optimized and CpG-depleted, and the multidomain therapeutic protein comprises the sequence set forth in SEQ ID NO: 193, optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 736, the coding sequence for the multidomain therapeutic protein is codon-optimized and CpG-depleted, and the multidomain therapeutic protein comprises the sequence set forth in SEQ ID NO: 193. In some such methods, the coding sequence for the multidomain therapeutic protein comprises the sequence set forth in any one of SEQ ID NOS: 194-202, optionally wherein the coding sequence for the multidomain therapeutic protein comprises the sequence set forth in SEQ ID NO: 196, optionally wherein the nucleic acid construct comprises the sequence set forth in SEQ ID NO: 736. In some such methods, the coding sequence for the multidomain therapeutic protein consists of the sequence set forth in any one of SEQ ID NOS: 194-202, optionally wherein the coding sequence for the multidomain therapeutic protein consists of the sequence set forth in SEQ ID NOS: 196, optionally wherein the nucleic acid construct comprises the sequence set forth in SEQ ID NO: 736.

In some such methods, the nucleic acid construct comprises a splice acceptor upstream of the coding sequence for the multidomain therapeutic protein. In some such methods, the nucleic acid construct comprises a polyadenylation signal or sequence downstream of the coding sequence for the multidomain therapeutic protein. In some such methods, the nucleic acid construct comprises a splice acceptor upstream of the coding sequence for the multidomain therapeutic protein, and the nucleic acid construct comprises a polyadenylation signal or sequence downstream of the coding sequence for the multidomain therapeutic protein. In some such methods, the nucleic acid construct does not comprise a homology arm. In some such methods, the nucleic acid construct is inserted into the target genomic locus via non-homologous end joining. In some such methods, the nucleic acid construct comprises homology arms. In some such methods, the nucleic acid construct is inserted into the target genomic locus via homology-directed repair. In some such methods, the nucleic acid construct does not comprise a promoter that drives the expression of the multidomain therapeutic protein. In some such methods, the coding sequence for the multidomain therapeutic protein is operably linked to a promoter, optionally wherein the promoter is a liver-specific promoter. In some such methods, the nucleic acid construct is single-stranded DNA or double-stranded DNA. In some such methods, the nucleic acid construct is single-stranded DNA. In some such methods, the nucleic acid construct comprises from 5′ to 3′: a splice acceptor, the coding sequence for the multidomain therapeutic protein, and a polyadenylation signal or sequence, wherein the coding sequence for the multidomain therapeutic protein comprises any one of SEQ ID NOS: 194-202, optionally wherein the coding sequence for the multidomain therapeutic protein comprises the sequence set forth in SEQ ID NO: 196, optionally wherein the nucleic acid construct comprises the sequence set forth in SEQ ID NO: 736, wherein the nucleic acid construct does not comprise a promoter that drives the expression of the multidomain therapeutic protein, and wherein the nucleic acid construct does not comprise a homology arm.

In some such methods, the nucleic acid construct is in a nucleic acid vector or a lipid nanoparticle. In some such methods, the nucleic acid construct is in the nucleic acid vector. In some such methods, the nucleic acid vector is a viral vector. In some such methods, the nucleic acid vector is an adeno-associated viral (AAV) vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160. In some such methods, the AAV vector is a single-stranded AAV (ssAAV) vector. In some such methods, the AAV vector is derived from an AAV8 vector, an AAV3B vector, an AAV5 vector, an AAV6 vector, an AAV7 vector, an AAV9 vector, an AAVrh.74 vector, or an AAVhu.37 vector. In some such methods, the AAV vector is a recombinant AAV8 (rAAV8) vector. In some such methods, the AAV vector is a single-stranded rAAV8 vector. In some such methods, the nucleic acid construct comprises from 5′ to 3′: a splice acceptor, the coding sequence for the multidomain therapeutic protein, and a polyadenylation signal or sequence, wherein the coding sequence for the multidomain therapeutic protein comprises any one of SEQ ID NOS: 194-202, optionally wherein the coding sequence for the multidomain therapeutic protein comprises the sequence set forth in SEQ ID NO: 196, optionally wherein the nucleic acid construct comprises the sequence set forth in SEQ ID NO: 736, wherein the nucleic acid construct does not comprise a promoter that drives the expression of the multidomain therapeutic protein, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160. In some such methods, the nucleic acid construct is CpG-depleted.

In some such methods, the target genomic locus is an albumin gene, optionally wherein the albumin gene is a human albumin gene. In some such methods, the nuclease target site is in intron 1 of the albumin gene. In some such methods, the nuclease agent comprises: (a) a zinc finger nuclease (ZFN); (b) a transcription activator-like effector nuclease (TALEN); or (c) (i) a Cas protein or a nucleic acid encoding the Cas protein; and (ii) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

In some such methods, the nuclease agent comprises: (a) a Cas protein or a nucleic acid encoding the Cas protein; and (b) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence. In some such methods, the guide RNA target sequence is in intron 1 of an albumin gene. In some such methods, the albumin gene is a human albumin gene. In some such methods, the DNA-targeting segment comprises at least 17, at least 18, at least 19, or at least 20 contiguous nucleotides of the sequence set forth in any one of SEQ ID NOS: 30-61, optionally wherein the DNA-targeting segment comprises at least 17, at least 18, at least 19, or at least 20 contiguous nucleotides of the sequence set forth in any one of SEQ ID NOS: 36, 30, 33, and 41. In some such methods, the DNA-targeting segment is at least 90% or at least 95% identical to the sequence set forth in any one of SEQ ID NOS: 30-61, optionally wherein the DNA-targeting segment is at least 90% or at least 95% identical to the sequence set forth in any one of SEQ ID NOS: 36, 30, 33, and 41. In some such methods, the DNA-targeting segment comprises any one of SEQ ID NOS: 30-61, optionally wherein the DNA-targeting segment comprises any one of SEQ ID NOS: 36, 30, 33, and 41. In some such methods, the DNA-targeting segment consists of any one of SEQ ID NOS: 30-61, optionally wherein the DNA-targeting segment consists of any one of SEQ ID NOS: 36, 30, 33, and 41. In some such methods, the guide RNA comprises any one of SEQ ID NOS: 62-125, optionally wherein the guide RNA comprises any one of SEQ ID NOS: 68, 100, 62, 94, 65, 97, 73, and 105. In some such methods, the DNA-targeting segment comprises at least 17, at least 18, at least 19, or at least 20 contiguous nucleotides of SEQ ID NO: 36. In some such methods, the DNA-targeting segment is at least 90% or at least 95% identical to SEQ ID NO: 36. In some such methods, the DNA-targeting segment comprises SEQ ID NO: 36. In some such methods, the DNA-targeting segment consists of SEQ ID NO: 36. In some such methods, the guide RNA comprises SEQ ID NO: 68 or 100.

In some such methods, the method comprises administering the guide RNA in the form of RNA. In some such methods, the guide RNA comprises at least one modification. In some such methods, the at least one modification comprises a 2′-O-methyl-modified nucleotide. In some such methods, the at least one modification comprises a phosphorothioate bond between nucleotides. In some such methods, the at least one modification comprises a modification at one or more of the first five nucleotides at the 5′ end of the guide RNA. In some such methods, the at least one modification comprises a modification at one or more of the last five nucleotides at the 3′ end of the guide RNA. In some such methods, the at least one modification comprises phosphorothioate bonds between the first four nucleotides at the 5′ end of the guide RNA. In some such methods, the at least one modification comprises phosphorothioate bonds between the last four nucleotides at the 3′ end of the guide RNA. In some such methods, the at least one modification comprises 2′-O-methyl-modified nucleotides at the first three nucleotides at the 5′ end of the guide RNA. In some such methods, the at least one modification comprises 2′-O-methyl-modified nucleotides at the last three nucleotides at the 3′ end of the guide RNA. In some such methods, the at least one modification comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA. In some such methods, the guide RNA is a single guide RNA (sgRNA). In some such methods, the method comprises administering the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

In some such methods, the Cas protein is a Cas9 protein. In some such methods, the Cas9 protein is derived from a Streptococcus pyogenes Cas9 protein, a Staphylococcus aureus Cas9 protein, a Campylobacter jejuni Cas9 protein, a Streptococcus thermophilus Cas9 protein, or a Neisseria meningitidis Cas9 protein. In some such methods, the Cas protein is derived from a Streptococcus pyogenes Cas9 protein. In some such methods, the Cas protein comprises the sequence set forth in SEQ ID NO: 11. In some such methods, the nucleic acid encoding the Cas protein is codon-optimized for expression in a mammalian cell or a human cell. In some such methods, the method comprises administering the nucleic acid encoding the Cas protein, wherein the nucleic acid comprises an mRNA encoding the Cas protein. In some such methods, the mRNA encoding the Cas protein comprises at least one modification. In some such methods, the mRNA encoding the Cas protein is modified to comprise a modified uridine at one or more or all uridine positions. In some such methods, the modified uridine is pseudouridine or N1-methyl-pseudouridine, optionally N1-methyl-pseudouridine. In some such methods, the mRNA encoding the Cas protein is fully substituted with pseudouridine or N1-methyl-pseudouridine, optionally N1-methyl-pseudouridine. In some such methods, the modified uridine is pseudouridine. In some such methods, the mRNA encoding the Cas protein is fully substituted with pseudouridine. In some such methods, the mRNA encoding the Cas protein comprises a 5′ cap. In some such methods, the mRNA encoding the Cas protein comprises a polyadenylation sequence. In some such methods, the mRNA encoding the Cas protein comprises the sequence set forth in SEQ ID NO: 226, 225, or 12. In some such methods, the method comprises administering the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine or N1-methyl-pseudouridine, optionally N1-methyl-pseudouridine, comprises a 5′ cap, and comprises a polyadenylation sequence. In some such methods, the method comprises administering the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine, comprises a 5′ cap, and comprises a polyadenylation sequence.

In some such methods, the method comprises administering the guide RNA in the form of RNA, and the guide RNA comprises SEQ ID NO: 68 or 100, and wherein the method comprises administering the nucleic acid encoding the Cas protein, wherein the nucleic acid comprises an mRNA encoding the Cas protein, and the mRNA encoding the Cas protein comprises the sequence set forth in SEQ ID NO: 226, 225, or 12. In some such methods, the nucleic acid construct comprises from 5′ to 3′: a splice acceptor, the coding sequence for the multidomain therapeutic protein, and a polyadenylation signal or sequence, wherein the coding sequence for the multidomain therapeutic protein comprises any one of SEQ ID NOS: 194-202, optionally wherein the coding sequence for the multidomain therapeutic protein comprises the sequence set forth in SEQ ID NO: 196, optionally wherein the nucleic acid construct comprises the sequence set forth in SEQ ID NO: 736, wherein the nucleic acid construct does not comprise a promoter that drives the expression of the multidomain therapeutic protein, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160. In some such methods, the method comprises administering the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the method comprises administering the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine or N1-methyl-pseudouridine, optionally N1-methyl-pseudouridine, comprises a 5′ cap, and comprises a polyadenylation sequence. In some such methods, the nucleic acid construct comprises from 5′ to 3′: a splice acceptor, the coding sequence for the multidomain therapeutic protein, and a polyadenylation signal or sequence, wherein the coding sequence for the multidomain therapeutic protein comprises any one of SEQ ID NOS: 194-202, optionally wherein the coding sequence for the multidomain therapeutic protein comprises the sequence set forth in SEQ ID NO: 196, optionally wherein the nucleic acid construct comprises the sequence set forth in SEQ ID NO: 736, wherein the nucleic acid construct does not comprise a promoter that drives the expression of the multidomain therapeutic protein, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160. In some such methods, the method comprises administering the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the method comprises administering the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine, comprises a 5′ cap, and comprises a polyadenylation sequence. In some such methods, the nucleic acid construct comprises from 5′ to 3′: a splice acceptor, the coding sequence for the multidomain therapeutic protein, and a polyadenylation signal or sequence, wherein the coding sequence for the multidomain therapeutic protein comprises any one of SEQ ID NOS: 194-202, optionally wherein the coding sequence for the multidomain therapeutic protein comprises the sequence set forth in SEQ ID NO: 196, optionally wherein the nucleic acid construct comprises the sequence set forth in SEQ ID NO: 736, wherein the nucleic acid construct does not comprise a promoter that drives the expression of the multidomain therapeutic protein, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

In some such methods, the Cas protein or the nucleic acid encoding the Cas protein and the guide RNA or the one or more DNAs encoding the guide RNA are associated with a lipid nanoparticle. In some such methods, the lipid nanoparticle comprises a cationic lipid, a neutral lipid, a helper lipid, and a stealth lipid. In some such methods, the cationic lipid is Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate). In some such methods, the neutral lipid is distearoylphosphatidylcholine or 1,2-distearoyl-sn-glycero-3-phosphocholine (DSPC). In some such methods, the helper lipid is cholesterol. In some such methods, the stealth lipid is PEG2k-DMG. In some such methods, the cationic lipid is Lipid A, the neutral lipid is DSPC, the helper lipid is cholesterol, and the stealth lipid is PEG2k-DMG. In some such methods, the lipid nanoparticle comprises four lipids at the following molar ratios: about 50 mol% Lipid A, about 9 mol% DSPC, about 38 mol% cholesterol, and about 3 mol% PEG2k-DMG.

In some such methods, the albumin gene is a human albumin gene, wherein the method comprises administering the guide RNA in the form of RNA, and the guide RNA comprises SEQ ID NO: 68 or 100, wherein the method comprises administering the nucleic acid encoding the Cas protein, wherein the nucleic acid comprises an mRNA encoding the Cas protein, and the mRNA encoding the Cas protein comprises the sequence set forth in SEQ ID NO: 226, 225, or 12, and wherein the guide RNA and the mRNA encoding the Cas protein are associated with a lipid nanoparticle comprising Lipid A, DSPC, cholesterol, and PEG2k-DMG, optionally at the following molar ratios: about 50 mol% Lipid A, about 9 mol% DSPC, about 38 mol% cholesterol, and about 3 mol% PEG2k-DMG. In some such methods, the nucleic acid construct comprises from 5′ to 3′: a splice acceptor, the coding sequence for the multidomain therapeutic protein, and a polyadenylation signal or sequence, wherein the coding sequence for the multidomain therapeutic protein comprises any one of SEQ ID NOS: 194-202, optionally wherein the coding sequence for the multidomain therapeutic protein comprises the sequence set forth in SEQ ID NO: 196, optionally wherein the nucleic acid construct comprises the sequence set forth in SEQ ID NO: 736, wherein the nucleic acid construct does not comprise a promoter that drives the expression of the multidomain therapeutic protein, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

In some such methods, the albumin gene is a human albumin gene, wherein the method comprises administering the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the method comprises administering the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine or N1-methyl-pseudouridine, optionally N1-methyl-pseudouridine, comprises a 5′ cap, and comprises a polyadenylation sequence, and wherein the guide RNA and the mRNA encoding the Cas protein are associated with a lipid nanoparticle comprising Lipid A, DSPC, cholesterol, and PEG2k-DMG, optionally at the following molar ratios: about 50 mol% Lipid A, about 9 mol% DSPC, about 38 mol% cholesterol, and about 3 mol% PEG2k-DMG. In some such methods, the albumin gene is a human albumin gene, wherein the method comprises administering the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the method comprises administering the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine, comprises a 5′ cap, and comprises a polyadenylation sequence, and wherein the guide RNA and the mRNA encoding the Cas protein are associated with a lipid nanoparticle comprising Lipid A, DSPC, cholesterol, and PEG2k-DMG, optionally at the following molar ratios: about 50 mol% Lipid A, about 9 mol% DSPC, about 38 mol% cholesterol, and about 3 mol% PEG2k-DMG. In some such methods, the nucleic acid construct comprises from 5′ to 3′: a splice acceptor, the coding sequence for the multidomain therapeutic protein, and a polyadenylation signal or sequence, wherein the coding sequence for the multidomain therapeutic protein comprises any one of SEQ ID NOS: 194-202, optionally wherein the coding sequence for the multidomain therapeutic protein comprises the sequence set forth in SEQ ID NO: 196, optionally wherein the nucleic acid construct comprises the sequence set forth in SEQ ID NO: 736, wherein the nucleic acid construct does not comprise a promoter that drives the expression of the multidomain therapeutic protein, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

In another aspect, provided are a neonatal cell or a population of neonatal cells made by any of the above methods. In another aspect, provided are a cell or a population of cells made by any of the above methods. In another aspect, provided are a neonatal cell or a population of neonatal cells comprising a nucleic acid construct inserted into a target genomic locus, wherein the nucleic acid construct comprises a coding sequence for a multidomain therapeutic protein comprising a delivery domain fused to a lysosomal alpha-glucosidase inserted into a target genomic locus, optionally wherein the delivery domain is a CD63-binding delivery domain or a TfR-binding delivery domain. In another aspect, provided are a neonatal cell or a population of neonatal cells comprising a nucleic acid construct inserted into a target genomic locus, wherein the nucleic acid construct comprises a coding sequence for a multidomain therapeutic protein comprising a CD63-binding delivery domain fused to a lysosomal alpha-glucosidase inserted into a target genomic locus. In some such neonatal cells or populations of neonatal cells, the neonatal cell is a liver cell or the population of neonatal cells is a population of liver cells. In some such neonatal cells or populations of neonatal cells, the neonatal cell is a hepatocyte or the population of neonatal cells is a population of hepatocytes. In some such neonatal cells or populations of neonatal cells, the neonatal cell is a human cell or the population of neonatal cells is a population of human cells. In some such neonatal cells or populations of neonatal cells, the neonatal cell or the population of neonatal cells is from a human neonatal subject within 24 weeks after birth. In some such neonatal cells or populations of neonatal cells, the neonatal cell or the population of neonatal cells is from a human neonatal subject within 12 weeks after birth. In some such neonatal cells or populations of neonatal cells, the neonatal cell or the population of neonatal cells is from a human neonatal subject within 8 weeks after birth. In some such neonatal cells or populations of neonatal cells, the neonatal cell or the population of neonatal cells is from a human neonatal subject within 4 weeks after birth. In some such neonatal cells or populations of neonatal cells, the neonatal cell is in vitro or ex vivo or the population of neonatal cells is in vitro or ex vivo. In some such neonatal cells or populations of neonatal cells, the neonatal cell is in vivo in a subject or the population of neonatal cells is in vivo. In some such neonatal cells or populations of neonatal cells, the multidomain therapeutic protein is expressed.

In some such neonatal cells or populations of neonatal cells, the CD63-binding delivery domain is fused to the lysosomal alpha-glucosidase protein via a peptide linker. In some such neonatal cells or populations of neonatal cells, the coding sequence for the CD63-binding delivery domain is codon-optimized or CpG-depleted. In some such neonatal cells or populations of neonatal cells, the coding sequence for the CD63-binding delivery domain is codon-optimized and CpG-depleted. In some such neonatal cells or populations of neonatal cells, the CD63-binding delivery domain comprises an anti-CD63 antigen-binding protein. In some such neonatal cells or populations of neonatal cells, the CD63-binding delivery domain comprises an anti-CD63 antibody, antibody fragment, or single-chain variable fragment (scFv). In some such neonatal cells or populations of neonatal cells, the CD63-binding delivery domain is the single-chain variable fragment (scFv). In some such neonatal cells or populations of neonatal cells, the scFv comprises the sequence set forth in SEQ ID NO: 183. In some such neonatal cells or populations of neonatal cells, the scFv consists of the sequence set forth in SEQ ID NO: 183. In some such neonatal cells or populations of neonatal cells, the scFv coding sequence is at least 90%, at least 91%, at least 92%, at least 93%, at least 94%, at least 95%, at least 96%, at least 97%, at least 98%, or at least 99% identical to any one of SEQ ID NOS: 184-192, optionally wherein the scFv coding sequence is at least 90%, at least 91%, at least 92%, at least 93%, at least 94%, at least 95%, at least 96%, at least 97%, at least 98%, or at least 99% identical to SEQ ID NO: 186. In some such neonatal cells or populations of neonatal cells, the scFv coding sequence is at least 90%, at least 91%, at least 92%, at least 93%, at least 94%, at least 95%, at least 96%, at least 97%, at least 98%, or at least 99% identical to any one of SEQ ID NOS: 184-192 and encodes an scFv comprising SEQ ID NO: 183, optionally wherein the scFv coding sequence is at least 90%, at least 91%, at least 92%, at least 93%, at least 94%, at least 95%, at least 96%, at least 97%, at least 98%, or at least 99% identical to SEQ ID NO: 186 and encodes an scFv comprising SEQ ID NO: 183. In some such neonatal cells or populations of neonatal cells, the scFv coding sequence is at least 90%, at least 91%, at least 92%, at least 93%, at least 94%, at least 95%, at least 96%, at least 97%, at least 98%, or at least 99% identical to any one of SEQ ID NOS: 184-192, is codon-optimized and CpG-depleted, and encodes an scFv comprising SEQ ID NO: 183, optionally wherein the scFv coding sequence is at least 90%, at least 91%, at least 92%, at least 93%, at least 94%, at least 95%, at least 96%, at least 97%, at least 98%, or at least 99% identical to SEQ ID NO: 186, is codon-optimized and CpG-depleted, and encodes an scFv comprising SEQ ID NO: 183. In some such neonatal cells or populations of neonatal cells, the scFv coding sequence comprises the sequence set forth in any one of SEQ ID NOS: 184-192, optionally wherein the scFv coding sequence comprises the sequence set forth in SEQ ID NO: 186. In some such neonatal cells or populations of neonatal cells, the scFv coding sequence consists of the sequence set forth in any one of SEQ ID NOS: 184-192, optionally wherein the scFv coding sequence consists of the sequence set forth in SEQ ID NO: 186.

In some such neonatal cells or populations of neonatal cells, the lysosomal alpha-glucosidase lacks the lysosomal alpha-glucosidase signal peptide and propeptide. In some such neonatal cells or populations of neonatal cells, the lysosomal alpha-glucosidase comprises the sequence set forth in SEQ ID NO: 173. In some such neonatal cells or populations of neonatal cells, the lysosomal alpha-glucosidase consists of the sequence set forth in SEQ ID NO: 173. In some such neonatal cells or populations of neonatal cells, the lysosomal alpha-glucosidase coding sequence is codon-optimized or CpG-depleted. In some such neonatal cells or populations of neonatal cells, the lysosomal alpha-glucosidase coding sequence is codon-optimized and CpG-depleted. In some such neonatal cells or populations of neonatal cells, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and 205-212, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176. In some such neonatal cells or populations of neonatal cells, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and 205-212 and encodes a lysosomal alpha-glucosidase protein comprising SEQ ID NO: 173, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176 and encodes a lysosomal alpha-glucosidase protein comprising SEQ ID NO: 173. In some such neonatal cells or populations of neonatal cells, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and 205-212, is codon-optimized and CpG-depleted, and encodes a lysosomal alpha-glucosidase protein comprising SEQ ID NO: 173, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176, is codon-optimized and CpG-depleted, and encodes a lysosomal alpha-glucosidase protein comprising SEQ ID NO: 173. In some such neonatal cells or populations of neonatal cells, the lysosomal alpha-glucosidase coding sequence comprises the sequence set forth in any one of SEQ ID NOS: 174-182 and 205-212, optionally wherein the lysosomal alpha-glucosidase coding sequence comprises the sequence set forth in SEQ ID NO: 176. In some such neonatal cells or populations of neonatal cells, the lysosomal alpha-glucosidase coding sequence consists of the sequence set forth in any one of SEQ ID NOS: 174-182 and 205-212, optionally wherein the lysosomal alpha-glucosidase coding sequence consists of the sequence set forth in SEQ ID NO: 176.

In some such neonatal cells or populations of neonatal cells, the lysosomal alpha-glucosidase lacks the lysosomal alpha-glucosidase signal peptide and propeptide. In some such neonatal cells or populations of neonatal cells, the lysosomal alpha-glucosidase comprises the sequence set forth in SEQ ID NO: 173. In some such neonatal cells or populations of neonatal cells, the lysosomal alpha-glucosidase consists of the sequence set forth in SEQ ID NO: 173. In some such neonatal cells or populations of neonatal cells, the lysosomal alpha-glucosidase coding sequence is codon-optimized or CpG-depleted. In some such neonatal cells or populations of neonatal cells, the lysosomal alpha-glucosidase coding sequence is codon-optimized and CpG-depleted. In some such neonatal cells or populations of neonatal cells, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176. In some such neonatal cells or populations of neonatal cells, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and encodes a lysosomal alpha-glucosidase protein comprising SEQ ID NO: 173, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176 and encodes a lysosomal alpha-glucosidase protein comprising SEQ ID NO: 173. In some such neonatal cells or populations of neonatal cells, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182, is codon-optimized and CpG-depleted, and encodes a lysosomal alpha-glucosidase protein comprising SEQ ID NO: 173, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176, is codon-optimized and CpG-depleted, and encodes a lysosomal alpha-glucosidase protein comprising SEQ ID NO: 173. In some such neonatal cells or populations of neonatal cells, the lysosomal alpha-glucosidase coding sequence comprises the sequence set forth in any one of SEQ ID NOS: 174-182, optionally wherein the lysosomal alpha-glucosidase coding sequence comprises the sequence set forth in SEQ ID NO: 176. In some such neonatal cells or populations of neonatal cells, the lysosomal alpha-glucosidase coding sequence consists of the sequence set forth in any one of SEQ ID NOS: 174-182, optionally wherein the lysosomal alpha-glucosidase coding sequence consists of the sequence set forth in SEQ ID NO: 176.

In some such neonatal cells or populations of neonatal cells, the coding sequence for the multidomain therapeutic protein is codon-optimized or CpG-depleted. In some such neonatal cells or populations of neonatal cells, the coding sequence for the multidomain therapeutic protein is codon-optimized and CpG-depleted. In some such neonatal cells or populations of neonatal cells, the multidomain therapeutic protein comprises the sequence set forth in SEQ ID NO: 193. In some such neonatal cells or populations of neonatal cells, the multidomain therapeutic protein consists of the sequence set forth in SEQ ID NO: 193. In some such neonatal cells or populations of neonatal cells, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 194-202, optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 196, optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 736. In some such neonatal cells or populations of neonatal cells, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 194-202, and the multidomain therapeutic protein comprises the sequence set forth in SEQ ID NO: 193, optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 196, and the multidomain therapeutic protein comprises the sequence set forth in SEQ ID NO: 193, optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 736, and the multidomain therapeutic protein comprises the sequence set forth in SEQ ID NO: 193. In some such neonatal cells or populations of neonatal cells, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 194-202 and is codon-optimized and CpG-depleted, and the multidomain therapeutic protein comprises the sequence set forth in SEQ ID NO: 193, optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 196 and is codon-optimized and CpG-depleted, and the multidomain therapeutic protein comprises the sequence set forth in SEQ ID NO: 193, optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 736, the coding sequence for the multidomain therapeutic protein is codon-optimized and CpG-depleted, and the multidomain therapeutic protein comprises the sequence set forth in SEQ ID NO: 193. In some such neonatal cells or populations of neonatal cells, the coding sequence for the multidomain therapeutic protein comprises the sequence set forth in any one of SEQ ID NOS: 194-202, optionally wherein the coding sequence for the multidomain therapeutic protein comprises the sequence set forth in SEQ ID NO: 196, optionally wherein the nucleic acid construct comprises the sequence set forth in SEQ ID NO: 736. In some such neonatal cells or populations of neonatal cells, the coding sequence for the multidomain therapeutic protein consists of the sequence set forth in any one of SEQ ID NOS: 194-202, optionally wherein the coding sequence for the multidomain therapeutic protein consists of the sequence set forth in SEQ ID NO: 196, optionally wherein the nucleic acid construct comprises the sequence set forth in SEQ ID NO: 736.

In some such neonatal cells or populations of neonatal cells, the nucleic acid construct comprises a splice acceptor upstream of the coding sequence for the multidomain therapeutic protein. In some such neonatal cells or populations of neonatal cells, the nucleic acid construct comprises a polyadenylation signal or sequence downstream of the coding sequence for the multidomain therapeutic protein. In some such neonatal cells or populations of neonatal cells, the nucleic acid construct comprises a splice acceptor upstream of the coding sequence for the multidomain therapeutic protein, and the nucleic acid construct comprises a polyadenylation signal or sequence downstream of the coding sequence for the multidomain therapeutic protein. In some such neonatal cells or populations of neonatal cells, the nucleic acid construct does not comprise a promoter that drives the expression of the multidomain therapeutic protein, and wherein the coding sequence for the multidomain therapeutic protein is operably linked to an endogenous promoter at the target genomic locus. In some such neonatal cells or populations of neonatal cells, the coding sequence for the multidomain therapeutic protein is operably linked to a promoter, optionally wherein the promoter is a liver-specific promoter. In some such neonatal cells or populations of neonatal cells, the nucleic acid construct comprises from 5′ to 3′: a splice acceptor, the coding sequence for the multidomain therapeutic protein, and a polyadenylation signal or sequence, wherein the coding sequence for the multidomain therapeutic protein comprises any one of SEQ ID NOS: 194-202, optionally wherein the coding sequence for the multidomain therapeutic protein comprises the sequence set forth in SEQ ID NO: 196, optionally wherein the nucleic acid construct comprises the sequence set forth in SEQ ID NO: 736, wherein the nucleic acid construct does not comprise a promoter that drives the expression of the multidomain therapeutic protein, and wherein the coding sequence for the multidomain therapeutic protein is operably linked to an endogenous promoter at the target genomic locus.

In some such neonatal cells or populations of neonatal cells, the target genomic locus is an albumin gene, optionally wherein the albumin gene is a human albumin gene. In some such neonatal cells or populations of neonatal cells, the nuclease target site is in intron 1 of the albumin gene.

In another aspect, provided are compositions comprising a nucleic acid construct comprising a coding sequence for a multidomain therapeutic protein comprising a delivery domain fused to a lysosomal alpha-glucosidase, wherein the lysosomal alpha-glucosidase coding sequence is CpG-depleted relative to a wild type lysosomal alpha-glucosidase coding sequence, optionally wherein the delivery domain is a CD63-binding delivery domain or a TfR-binding delivery domain. In another aspect, provided are compositions comprising a nucleic acid construct comprising a coding sequence for a multidomain therapeutic protein comprising a CD63-binding delivery domain fused to a lysosomal alpha-glucosidase, wherein the lysosomal alpha-glucosidase coding sequence is CpG-depleted relative to a wild type lysosomal alpha-glucosidase coding sequence. In some such compositions, the CD63-binding delivery domain is fused to the lysosomal alpha-glucosidase protein via a peptide linker. In some such compositions, the lysosomal alpha-glucosidase lacks the lysosomal alpha-glucosidase signal peptide and propeptide. In some such compositions, the lysosomal alpha-glucosidase comprises the sequence set forth in SEQ ID NO: 173. In some such compositions, the lysosomal alpha-glucosidase consists of the sequence set forth in SEQ ID NO: 173. In some such compositions, the lysosomal alpha-glucosidase coding sequence is codon-optimized and CpG-depleted. In some such compositions, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and 205-212, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176. In some such compositions, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and 205-212 and encodes a lysosomal alpha-glucosidase protein comprising SEQ ID NO: 173, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176 and encodes a lysosomal alpha-glucosidase protein comprising SEQ ID NO: 173. In some such compositions, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and 205-212, is codon-optimized and CpG-depleted, and encodes a lysosomal alpha-glucosidase protein comprising SEQ ID NO: 173, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176, is codon-optimized and CpG-depleted, and encodes a lysosomal alpha-glucosidase protein comprising SEQ ID NO: 173. In some such compositions, the lysosomal alpha-glucosidase coding sequence comprises the sequence set forth in any one of SEQ ID NOS: 174-182 and 205-212, optionally wherein the lysosomal alpha-glucosidase coding sequence comprises the sequence set forth in SEQ ID NO: 176. In some such compositions, the lysosomal alpha-glucosidase coding sequence consists of the sequence set forth in any one of SEQ ID NOS: 174-182 and 205-212, optionally wherein the lysosomal alpha-glucosidase coding sequence consists of the sequence set forth in SEQ ID NO: 176.

In another aspect, provided are compositions comprising a nucleic acid construct comprising a coding sequence for a multidomain therapeutic protein comprising a CD63-binding delivery domain fused to a lysosomal alpha-glucosidase, wherein the lysosomal alpha-glucosidase coding sequence is CpG-depleted relative to a wild type lysosomal alpha-glucosidase coding sequence. In some such compositions, the CD63-binding delivery domain is fused to the lysosomal alpha-glucosidase protein via a peptide linker. In some such compositions, the lysosomal alpha-glucosidase lacks the lysosomal alpha-glucosidase signal peptide and propeptide. In some such compositions, the lysosomal alpha-glucosidase comprises the sequence set forth in SEQ ID NO: 173. In some such compositions, the lysosomal alpha-glucosidase consists of the sequence set forth in SEQ ID NO: 173. In some such compositions, the lysosomal alpha-glucosidase coding sequence is codon-optimized and CpG-depleted. In some such compositions, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176. In some such compositions, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and encodes a lysosomal alpha-glucosidase protein comprising SEQ ID NO: 173, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176 and encodes a lysosomal alpha-glucosidase protein comprising SEQ ID NO: 173. In some such compositions, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182, is codon-optimized and CpG-depleted, and encodes a lysosomal alpha-glucosidase protein comprising SEQ ID NO: 173, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176, is codon-optimized and CpG-depleted, and encodes a lysosomal alpha-glucosidase protein comprising SEQ ID NO: 173. In some such compositions, the lysosomal alpha-glucosidase coding sequence comprises the sequence set forth in any one of SEQ ID NOS: 174-182, optionally wherein the lysosomal alpha-glucosidase coding sequence comprises the sequence set forth in SEQ ID NO: 176. In some such compositions, the lysosomal alpha-glucosidase coding sequence consists of the sequence set forth in any one of SEQ ID NOS: 174-182, optionally wherein the lysosomal alpha-glucosidase coding sequence consists of the sequence set forth in SEQ ID NO: 176.

In some such compositions, the coding sequence for the CD63-binding delivery domain is codon-optimized or CpG-depleted. In some such compositions, the coding sequence for the CD63-binding delivery domain is codon-optimized and CpG-depleted. In some such compositions, the CD63-binding delivery domain comprises an anti-CD63 antigen-binding protein. In some such compositions, the CD63-binding delivery domain comprises an anti-CD63 antibody, antibody fragment, or single-chain variable fragment (scFv). In some such compositions, the CD63-binding delivery domain is the single-chain variable fragment (scFv). In some such compositions, the scFv comprises the sequence set forth in SEQ ID NO: 183. In some such compositions, the scFv consists of the sequence set forth in SEQ ID NO: 183. In some such compositions, the scFv coding sequence is at least 90%, at least 91%, at least 92%, at least 93%, at least 94%, at least 95%, at least 96%, at least 97%, at least 98%, or at least 99% identical to any one of SEQ ID NOS: 184-192, optionally wherein the scFv coding sequence is at least 90%, at least 91%, at least 92%, at least 93%, at least 94%, at least 95%, at least 96%, at least 97%, at least 98%, or at least 99% identical to SEQ ID NO: 186. In some such compositions, the scFv coding sequence is at least 90%, at least 91%, at least 92%, at least 93%, at least 94%, at least 95%, at least 96%, at least 97%, at least 98%, or at least 99% identical to any one of SEQ ID NOS: 184-192 and encodes an scFv comprising SEQ ID NO: 183, optionally wherein the scFv coding sequence is at least 90%, at least 91%, at least 92%, at least 93%, at least 94%, at least 95%, at least 96%, at least 97%, at least 98%, or at least 99% identical to SEQ ID NO: 186 and encodes an scFv comprising SEQ ID NO: 183. In some such compositions, the scFv coding sequence is at least 90%, at least 91%, at least 92%, at least 93%, at least 94%, at least 95%, at least 96%, at least 97%, at least 98%, or at least 99% identical to any one of SEQ ID NOS: 184-192, is codon-optimized and CpG-depleted, and encodes an scFv comprising SEQ ID NO: 183, optionally wherein the scFv coding sequence is at least 90%, at least 91%, at least 92%, at least 93%, at least 94%, at least 95%, at least 96%, at least 97%, at least 98%, or at least 99% identical to SEQ ID NO: 186, is codon-optimized and CpG-depleted, and encodes an scFv comprising SEQ ID NO: 183. In some such compositions, the scFv coding sequence comprises the sequence set forth in any one of SEQ ID NOS: 184-192, optionally wherein the scFv coding sequence comprises the sequence set forth in SEQ ID NO: 186. In some such compositions, the scFv coding sequence consists of the sequence set forth in any one of SEQ ID NOS: 184-192, optionally wherein the scFv coding sequence consists of the sequence set forth in SEQ ID NO: 186.

In some such compositions, the coding sequence for the multidomain therapeutic protein is codon-optimized or CpG-depleted. In some such compositions, the coding sequence for the multidomain therapeutic protein is codon-optimized and CpG-depleted. In some such compositions, the multidomain therapeutic protein comprises the sequence set forth in SEQ ID NO: 193. In some such compositions, the multidomain therapeutic protein consists of the sequence set forth in SEQ ID NO: 193. In some such compositions, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 194-202, optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 196, optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 736. In some such compositions, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 194-202, and the multidomain therapeutic protein comprises the sequence set forth in SEQ ID NO: 193, optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 196, and the multidomain therapeutic protein comprises the sequence set forth in SEQ ID NO: 193, optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 736, and the multidomain therapeutic protein comprises the sequence set forth in SEQ ID NO: 193. In some such compositions, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 194-202 and is codon-optimized and CpG-depleted, and the multidomain therapeutic protein comprises the sequence set forth in SEQ ID NO: 193, optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 196 and is codon-optimized and CpG-depleted, and the multidomain therapeutic protein comprises the sequence set forth in SEQ ID NO: 193, optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 736, the coding sequence for the multidomain therapeutic protein is codon-optimized and CpG-depleted, and the multidomain therapeutic protein comprises the sequence set forth in SEQ ID NO: 193. In some such compositions, the coding sequence for the multidomain therapeutic protein comprises the sequence set forth in any one of SEQ ID NOS: 194-202, optionally wherein the coding sequence for the multidomain therapeutic protein comprises the sequence set forth in SEQ ID NO: 196, optionally wherein the nucleic acid construct comprises the sequence set forth in SEQ ID NO: 736. In some such compositions, the coding sequence for the multidomain therapeutic protein consists of the sequence set forth in any one of SEQ ID NOS: 194-202, optionally wherein the coding sequence for the multidomain therapeutic protein consists of the sequence set forth in SEQ ID NO: 196, optionally wherein the nucleic acid construct comprises the sequence set forth in SEQ ID NO: 736.

In some such compositions, the nucleic acid construct comprises a splice acceptor upstream of the coding sequence for the multidomain therapeutic protein. In some such compositions, the nucleic acid construct comprises a polyadenylation signal or sequence downstream of the coding sequence for the multidomain therapeutic protein. In some such compositions, the nucleic acid construct comprises a splice acceptor upstream of the coding sequence for the multidomain therapeutic protein, and the nucleic acid construct comprises a polyadenylation signal or sequence downstream of the coding sequence for the multidomain therapeutic protein. In some such compositions, the nucleic acid construct does not comprise a homology arm. In some such compositions, the nucleic acid construct comprises homology arms. In some such compositions, the nucleic acid construct does not comprise a promoter that drives the expression of the multidomain therapeutic protein. In some such compositions, the nucleic acid construct is single-stranded DNA or double-stranded DNA. In some such compositions, the nucleic acid construct is single-stranded DNA. In some such compositions, the nucleic acid construct comprises from 5′ to 3′: a splice acceptor, the coding sequence for the multidomain therapeutic protein, and a polyadenylation signal or sequence, wherein the coding sequence for the multidomain therapeutic protein comprises any one of SEQ ID NOS: 194-202, optionally wherein the coding sequence for the multidomain therapeutic protein comprises the sequence set forth in SEQ ID NO: 196, optionally wherein the nucleic acid construct comprises the sequence set forth in SEQ ID NO: 736, wherein the nucleic acid construct does not comprise a promoter that drives the expression of the multidomain therapeutic protein, and wherein the nucleic acid construct does not comprise a homology arm.

In some such compositions, the nucleic acid construct is in a nucleic acid vector or a lipid nanoparticle. In some such compositions, the nucleic acid construct is in the nucleic acid vector. In some such compositions, the nucleic acid vector is a viral vector. In some such compositions, the nucleic acid vector is an adeno-associated viral (AAV) vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160. In some such compositions, the AAV vector is a single-stranded AAV (ssAAV) vector. In some such compositions, the AAV vector is derived from an AAV8 vector, an AAV3B vector, an AAV5 vector, an AAV6 vector, an AAV7 vector, an AAV9 vector, an AAVrh.74 vector, or an AAVhu.37 vector. In some such compositions, the AAV vector is a recombinant AAV8 (rAAV8) vector. In some such compositions, the AAV vector is a single-stranded rAAV8 vector. In some such compositions, the nucleic acid construct comprises from 5′ to 3′: a splice acceptor, the coding sequence for the multidomain therapeutic protein, and a polyadenylation signal or sequence, wherein the coding sequence for the multidomain therapeutic protein comprises any one of SEQ ID NOS: 194-202, optionally wherein the coding sequence for the multidomain therapeutic protein comprises the sequence set forth in SEQ ID NO: 196, optionally wherein the nucleic acid construct comprises the sequence set forth in SEQ ID NO: 736, wherein the nucleic acid construct does not comprise a promoter that drives the expression of the multidomain therapeutic protein, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160. In some such compositions, the nucleic acid construct is CpG-depleted.

In some such compositions, the composition further comprise a nuclease agent that targets a nuclease target site in a target genomic locus. In some such compositions, the target genomic locus is an albumin gene, optionally wherein the albumin gene is a human albumin gene. In some such compositions, the nuclease target site is in intron 1 of the albumin gene. In some such compositions, the nuclease agent comprises: (a) a zinc finger nuclease (ZFN); (b) a transcription activator-like effector nuclease (TALEN); or (c) (i) a Cas protein or a nucleic acid encoding the Cas protein; and (ii) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

In some such compositions, the nuclease agent comprises: (a) a Cas protein or a nucleic acid encoding the Cas protein; and (b) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence. In some such compositions, the guide RNA target sequence is in intron 1 of an albumin gene. In some such compositions, the albumin gene is a human albumin gene. In some such compositions, the DNA-targeting segment comprises at least 17, at least 18, at least 19, or at least 20 contiguous nucleotides of the sequence set forth in any one of SEQ ID NOS: 30-61, optionally wherein the DNA-targeting segment comprises at least 17, at least 18, at least 19, or at least 20 contiguous nucleotides of the sequence set forth in any one of SEQ ID NOS: 36, 30, 33, and 41. In some such compositions, the DNA-targeting segment is at least 90% or at least 95% identical to the sequence set forth in any one of SEQ ID NOS: 30-61, optionally wherein the DNA-targeting segment is at least 90% or at least 95% identical to the sequence set forth in any one of SEQ ID NOS: 36, 30, 33, and 41. In some such compositions, the DNA-targeting segment comprises any one of SEQ ID NOS: 30-61, optionally wherein the DNA-targeting segment comprises any one of SEQ ID NOS: 36, 30, 33, and 41. In some such compositions, the DNA-targeting segment consists of any one of SEQ ID NOS: 30-61, optionally wherein the DNA-targeting segment consists of any one of SEQ ID NOS: 36, 30, 33, and 41. In some such compositions, the guide RNA comprises any one of SEQ ID NOS: 62-125, optionally wherein the guide RNA comprises any one of SEQ ID NOS: 68, 100, 62, 94, 65, 97, 73, and 105. In some such compositions, the DNA-targeting segment comprises at least 17, at least 18, at least 19, or at least 20 contiguous nucleotides of SEQ ID NO: 36. In some such compositions, the DNA-targeting segment is at least 90% or at least 95% identical to SEQ ID NO: 36. In some such compositions, the DNA-targeting segment comprises SEQ ID NO: 36. In some such compositions, the DNA-targeting segment consists of SEQ ID NO: 36. In some such compositions, the guide RNA comprises SEQ ID NO: 68 or 100.

In some such compositions, the guide RNA in the form of RNA. In some such compositions, the guide RNA comprises at least one modification. In some such compositions, the at least one modification comprises a 2′-O-methyl-modified nucleotide. In some such compositions, the at least one modification comprises a phosphorothioate bond between nucleotides. In some such compositions, the at least one modification comprises a modification at one or more of the first five nucleotides at the 5′ end of the guide RNA. In some such compositions, the at least one modification comprises a modification at one or more of the last five nucleotides at the 3′ end of the guide RNA. In some such compositions, the at least one modification comprises phosphorothioate bonds between the first four nucleotides at the 5′ end of the guide RNA. In some such compositions, the at least one modification comprises phosphorothioate bonds between the last four nucleotides at the 3′ end of the guide RNA. In some such compositions, the at least one modification comprises 2′-O-methyl-modified nucleotides at the first three nucleotides at the 5′ end of the guide RNA. In some such compositions, the at least one modification comprises 2′-O-methyl-modified nucleotides at the last three nucleotides at the 3′ end of the guide RNA. In some such compositions, the at least one modification comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA. In some such compositions, the guide RNA is a single guide RNA (sgRNA). In some such compositions, the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

In some such compositions, the Cas protein is a Cas9 protein. In some such compositions, the Cas9 protein is derived from a Streptococcus pyogenes Cas9 protein, a Staphylococcus aureus Cas9 protein, a Campylobacter jejuni Cas9 protein, a Streptococcus thermophilus Cas9 protein, or a Neisseria meningitidis Cas9 protein. In some such compositions, the Cas protein is derived from a Streptococcus pyogenes Cas9 protein. In some such compositions, the Cas protein comprises the sequence set forth in SEQ ID NO: 11. In some such compositions, the nucleic acid encoding the Cas protein is codon-optimized for expression in a mammalian cell or a human cell. In some such compositions, the composition comprises the nucleic acid encoding the Cas protein, wherein the nucleic acid comprises an mRNA encoding the Cas protein. In some such compositions, the mRNA encoding the Cas protein comprises at least one modification. In some such compositions, the mRNA encoding the Cas protein is modified to comprise a modified uridine at one or more or all uridine positions. In some such compositions, the modified uridine is pseudouridine or N1-methyl-pseudouridine, optionally N1-methyl-pseudouridine. In some such compositions, the mRNA encoding the Cas protein is fully substituted with pseudouridine or N1-methyl-pseudouridine, optionally N1-methyl-pseudouridine. In some such compositions, the modified uridine is pseudouridine. In some such compositions, the mRNA encoding the Cas protein is fully substituted with pseudouridine. In some such compositions, the mRNA encoding the Cas protein comprises a 5′ cap. In some such compositions, the mRNA encoding the Cas protein comprises a polyadenylation sequence. In some such compositions, the mRNA encoding the Cas protein comprises the sequence set forth in SEQ ID NO: 226, 225, or 12. In some such compositions, the composition comprises the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine or N1-methyl-pseudouridine, optionally N1-methyl-pseudouridine, comprises a 5′ cap, and comprises a polyadenylation sequence. In some such compositions, the composition comprises the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine, comprises a 5′ cap, and comprises a polyadenylation sequence.

In some such compositions, the guide RNA in the form of RNA, and the guide RNA comprises SEQ ID NO: 68 or 100, and wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 226, 225, or 12. In some such compositions, the nucleic acid construct comprises from 5′ to 3′: a splice acceptor, the coding sequence for the multidomain therapeutic protein, and a polyadenylation signal or sequence, wherein the coding sequence for the multidomain therapeutic protein comprises any one of SEQ ID NOS: 194-202, optionally wherein the coding sequence for the multidomain therapeutic protein comprises the sequence set forth in SEQ ID NO: 196, optionally wherein the nucleic acid construct comprises the sequence set forth in SEQ ID NO: 736, wherein the nucleic acid construct does not comprise a promoter that drives the expression of the multidomain therapeutic protein, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160. In some such compositions, the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine or N1-methyl-pseudouridine, optionally N1-methyl-pseudouridine, comprises a 5′ cap, and comprises a polyadenylation sequence. In some such compositions, the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine, comprises a 5′ cap, and comprises a polyadenylation sequence. In some such compositions, the nucleic acid construct comprises from 5′ to 3′: a splice acceptor, the coding sequence for the multidomain therapeutic protein, and a polyadenylation signal or sequence, wherein the coding sequence for the multidomain therapeutic protein comprises any one of SEQ ID NOS: 194-202, optionally wherein the coding sequence for the multidomain therapeutic protein comprises the sequence set forth in SEQ ID NO: 196, optionally wherein the nucleic acid construct comprises the sequence set forth in SEQ ID NO: 736, wherein the nucleic acid construct does not comprise a promoter that drives the expression of the multidomain therapeutic protein, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

In some such compositions, the Cas protein or the nucleic acid encoding the Cas protein and the guide RNA or the one or more DNAs encoding the guide RNA are associated with a lipid nanoparticle. In some such compositions, the lipid nanoparticle comprises a cationic lipid, a neutral lipid, a helper lipid, and a stealth lipid. In some such compositions, the cationic lipid is Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate). In some such compositions, the neutral lipid is distearoylphosphatidylcholine or 1,2-distearoyl-sn-glycero-3-phosphocholine (DSPC). In some such compositions, the helper lipid is cholesterol. In some such compositions, the stealth lipid is PEG2k-DMG. In some such compositions, the cationic lipid is Lipid A, the neutral lipid is DSPC, the helper lipid is cholesterol, and the stealth lipid is PEG2k-DMG. In some such compositions, the lipid nanoparticle comprises four lipids at the following molar ratios: about 50 mol% Lipid A, about 9 mol% DSPC, about 38 mol% cholesterol, and about 3 mol% PEG2k-DMG.

In some such compositions, the albumin gene is a human albumin gene, wherein the guide RNA in the form of RNA, and the guide RNA comprises SEQ ID NO: 68 or 100, wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 226, 225, or 12, and wherein the guide RNA and the mRNA encoding the Cas protein are associated with a lipid nanoparticle comprising Lipid A, DSPC, cholesterol, and PEG2k-DMG, optionally at the following molar ratios: about 50 mol% Lipid A, about 9 mol% DSPC, about 38 mol% cholesterol, and about 3 mol% PEG2k-DMG. In some such compositions, the nucleic acid construct comprises from 5′ to 3′: a splice acceptor, the coding sequence for the multidomain therapeutic protein, and a polyadenylation signal or sequence, wherein the coding sequence for the multidomain therapeutic protein comprises any one of SEQ ID NOS: 194-202, optionally wherein the coding sequence for the multidomain therapeutic protein comprises the sequence set forth in SEQ ID NO: 196, optionally wherein the nucleic acid construct comprises the sequence set forth in SEQ ID NO: 736, wherein the nucleic acid construct does not comprise a promoter that drives the expression of the multidomain therapeutic protein, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160. In some such compositions, the albumin gene is a human albumin gene, wherein the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine or N1-methyl-pseudouridine, optionally N1-methyl-pseudouridine, comprises a 5′ cap, and comprises a polyadenylation sequence, and wherein the guide RNA and the mRNA encoding the Cas protein are associated with a lipid nanoparticle comprising Lipid A, DSPC, cholesterol, and PEG2k-DMG, optionally at the following molar ratios: about 50 mol% Lipid A, about 9 mol% DSPC, about 38 mol% cholesterol, and about 3 mol% PEG2k-DMG. In some such compositions, the albumin gene is a human albumin gene, wherein the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine, comprises a 5′ cap, and comprises a polyadenylation sequence, and wherein the guide RNA and the mRNA encoding the Cas protein are associated with a lipid nanoparticle comprising Lipid A, DSPC, cholesterol, and PEG2k-DMG, optionally at the following molar ratios: about 50 mol% Lipid A, about 9 mol% DSPC, about 38 mol% cholesterol, and about 3 mol% PEG2k-DMG. In some such compositions, the nucleic acid construct comprises from 5′ to 3′: a splice acceptor, the coding sequence for the multidomain therapeutic protein, and a polyadenylation signal or sequence, wherein the coding sequence for the multidomain therapeutic protein comprises any one of SEQ ID NOS: 194-202, optionally wherein the coding sequence for the multidomain therapeutic protein comprises the sequence set forth in SEQ ID NO: 196, optionally wherein the nucleic acid construct comprises the sequence set forth in SEQ ID NO: 736, wherein the nucleic acid construct does not comprise a promoter that drives the expression of the multidomain therapeutic protein, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

In some such compositions, the composition is for use in a method of inserting the nucleic acid encoding the multidomain therapeutic protein into a target genomic locus in a cell or a population of cells. In some such compositions, the composition is for use in a method of expressing the multidomain therapeutic protein from a target genomic locus in a cell or a population of cells. In some such compositions, the composition is for use in a method of expressing the multidomain therapeutic protein in a cell or a population of cells. In some such compositions, the composition is for use in a method of inserting the nucleic acid encoding the multidomain therapeutic protein into a target genomic locus in a cell or a population of cells in a subject. In some such compositions, the composition is for use in a method of expressing the multidomain therapeutic protein from a target genomic locus in a cell or a population of cells in a subject. In some such compositions, the composition is for use in a method of expressing the multidomain therapeutic protein in a cell or a population of cells in a subject. In some such compositions, the cell is a neonatal cell and the population of cells is a population of neonatal cells. In some such compositions, the cell is a not a neonatal cell and the population of cells is not a population of neonatal cells. In some such compositions, the composition is for use in a method of treating a lysosomal alpha-glucosidase deficiency in a subject in need thereof. In some such compositions, the composition is for use in a method of reducing glycogen accumulation in a tissue in a subject in need thereof. In some such compositions, the composition is for use in a method of treating Pompe disease in a subject in need thereof. In some such compositions, the composition is for use in a method of preventing or reducing the onset of a sign or symptom of Pompe disease in a neonatal subject in need thereof. In some such compositions, the subject is a neonatal subject. In some such compositions, the subject is not a neonatal subject.

In another aspect, provided is a cell comprising any of the above compositions. In some such cells, the nucleic acid construct is integrated into a target genomic locus, and wherein the multidomain therapeutic protein is expressed from the target genomic locus, or wherein the nucleic acid construct is integrated into intron 1 of an endogenous albumin locus, and wherein the multidomain therapeutic protein is expressed from the endogenous albumin locus. In some such cells, the cell is a human cell. In some such cells, the cell is a liver cell. In some such cells, the liver cell is a hepatocyte. In some such cells, the cell is a neonatal cell. In some such cells, the neonatal cell is from a human neonatal subject within 24 weeks after birth. In some such cells, the neonatal cell is from a human neonatal subject within 12 weeks after birth. In some such cells, the neonatal cell is from a human neonatal subject within 8 weeks after birth. In some such cells, the neonatal cell is from a human neonatal subject within 4 weeks after birth. In some such cells, the cell is not a neonatal cell. In some such cells, the cell is in vivo. In some such cells, the cell is in vitro or ex vivo.

In another aspect, provided are methods of inserting a nucleic acid encoding a multidomain therapeutic protein comprising a delivery domain fused to a lysosomal alpha-glucosidase into a target genomic locus in a cell or a population of cells, optionally wherein the delivery domain is a CD63-binding delivery domain or a TfR-binding delivery domain. Some such methods comprise administering to the cell or the population of cells any of the above compositions, optionally wherein the delivery domain is a CD63-binding delivery domain or a TfR-binding delivery domain, wherein the nuclease agent cleaves the nuclease target site in the target genomic locus, and the nucleic acid construct is inserted into the target genomic locus. In another aspect, provided are methods of inserting a nucleic acid encoding a multidomain therapeutic protein comprising a CD63-binding delivery domain fused to a lysosomal alpha-glucosidase into a target genomic locus in a cell or a population of cells. Some such methods comprise administering to the cell or the population of cells any of the above compositions, wherein the nuclease agent cleaves the nuclease target site in the target genomic locus, and the nucleic acid construct is inserted into the target genomic locus. In another aspect, provided are methods of expressing a multidomain therapeutic protein comprising a delivery domain fused to a lysosomal alpha-glucosidase in a cell or a population of cells, optionally wherein the delivery domain is a CD63-binding delivery domain or a TfR-binding delivery domain. Some such methods comprise administering to the cell or the population of cells any of the above compositions, optionally wherein the delivery domain is a CD63-binding delivery domain or a TfR-binding delivery domain, wherein the coding sequence for the multidomain therapeutic protein is operably linked to a promoter in the nucleic acid construct and is expressed in the cell or population of cells. In another aspect, provided are methods of expressing a multidomain therapeutic protein comprising a delivery domain fused to a lysosomal alpha-glucosidase from a target genomic locus in a cell or a population of cells, optionally wherein the delivery domain is a CD63-binding delivery domain or a TfR-binding delivery domain. Some such methods comprise administering to the cell or the population of cells any of the above compositions, optionally wherein the delivery domain is a CD63-binding delivery domain or a TfR-binding delivery domain, wherein the nuclease agent cleaves the nuclease target site in the target genomic locus, the nucleic acid construct is inserted into the target genomic locus to create a modified target genomic locus, and the multidomain therapeutic protein comprising the delivery domain fused to the lysosomal alpha-glucosidase is expressed from the modified target genomic locus. In another aspect, provided are methods of expressing a multidomain therapeutic protein comprising a CD63-binding delivery domain fused to a lysosomal alpha-glucosidase in a cell or a population of cells. Some such methods comprise administering to the cell or the population of cells any of the above compositions, wherein the coding sequence for the multidomain therapeutic protein is operably linked to a promoter in the nucleic acid construct and is expressed in the cell or population of cells. In another aspect, provided are methods of expressing a multidomain therapeutic protein comprising a CD63-binding delivery domain fused to a lysosomal alpha-glucosidase from a target genomic locus in a cell or a population of cells. Some such methods comprise administering to the cell or the population of cells any of the above compositions, wherein the nuclease agent cleaves the nuclease target site in the target genomic locus, the nucleic acid construct is inserted into the target genomic locus to create a modified target genomic locus, and the multidomain therapeutic protein comprising the CD63-binding delivery domain fused to the lysosomal alpha-glucosidase is expressed from the modified target genomic locus. In some such methods, the cell is a liver cell or the population of cells is a population of liver cells. In some such methods, the cell is a hepatocyte or the population of cells is a population of hepatocytes. In some such methods, the cell is a human cell or the population of cells is a population of human cells. In some such methods, the cell is a neonatal cell or the population of cells is a population of neonatal cells. In some such methods, the neonatal cell or the population of neonatal cells is from a human neonatal subject within 24 weeks after birth. In some such methods, the neonatal cell or the population of neonatal cells is from a human neonatal subject within 12 weeks after birth. In some such methods, the neonatal cell or the population of neonatal cells is from a human neonatal subject within 8 weeks after birth. In some such methods, the neonatal cell or the population of neonatal cells is from a human neonatal subject within 4 weeks after birth. In some such methods, the cell is not a neonatal cell or the population of cells is not a population of neonatal cells. In some such methods, the cell is in vitro or ex vivo or the population of cells is in vitro or ex vivo. In some such methods, the cell is in vivo in a subject or the population of cells is in vivo in a subject.

In another aspect, provided are methods of inserting a nucleic acid encoding a multidomain therapeutic protein comprising a delivery domain fused to a lysosomal alpha-glucosidase into a target genomic locus in a cell or a population of cells in a subject, optionally wherein the delivery domain is a CD63-binding delivery domain or a TfR-binding delivery domain. Some such methods comprise administering to the subject any of the above compositions, optionally wherein the delivery domain is a CD63-binding delivery domain or a TfR-binding delivery domain, wherein the nuclease agent cleaves the nuclease target site in the target genomic locus, and the nucleic acid construct is inserted into the target genomic locus. In another aspect, provided are methods of inserting a nucleic acid encoding a multidomain therapeutic protein comprising a CD63-binding delivery domain fused to a lysosomal alpha-glucosidase into a target genomic locus in a cell or a population of cells in a subject. Some such methods comprise administering to the subject any of the above compositions, wherein the nuclease agent cleaves the nuclease target site in the target genomic locus, and the nucleic acid construct is inserted into the target genomic locus. In another aspect, provided are methods of expressing a multidomain therapeutic protein comprising a delivery domain fused to a lysosomal alpha-glucosidase protein in a cell or a population of cells in a subject, optionally wherein the delivery domain is a CD63-binding delivery domain or a TfR-binding delivery domain. Some such methods comprise administering to the subject any of the above compositions, optionally wherein the delivery domain is a CD63-binding delivery domain or a TfR-binding delivery domain, wherein the coding sequence for the multidomain therapeutic protein is operably linked to a promoter in the nucleic acid construct and is expressed in the cell. In another aspect, provided are methods of expressing a multidomain therapeutic protein comprising a delivery domain fused to a lysosomal alpha-glucosidase protein from a target genomic locus in a cell or a population of cells in a subject, optionally wherein the delivery domain is a CD63-binding delivery domain or a TfR-binding delivery domain. Some such methods comprise administering to the subject any of the above compositions, optionally wherein the delivery domain is a CD63-binding delivery domain or a TfR-binding delivery domain, wherein the nuclease agent cleaves the nuclease target site in the target genomic locus, the nucleic acid construct is inserted into the target genomic locus to create a modified target genomic locus, and the multidomain therapeutic protein comprising the delivery domain fused to the lysosomal alpha-glucosidase is expressed from the modified target genomic locus. In another aspect, provided are methods of expressing a multidomain therapeutic protein comprising a CD63-binding delivery domain fused to a lysosomal alpha-glucosidase protein in a cell or a population of cells in a subject. Some such methods comprise administering to the subject any of the above compositions, wherein the coding sequence for the multidomain therapeutic protein is operably linked to a promoter in the nucleic acid construct and is expressed in the cell. In another aspect, provided are methods of expressing a multidomain therapeutic protein comprising a CD63-binding delivery domain fused to a lysosomal alpha-glucosidase protein from a target genomic locus in a cell or a population of cells in a subject. Some such methods comprise administering to the subject any of the above compositions, wherein the nuclease agent cleaves the nuclease target site in the target genomic locus, the nucleic acid construct is inserted into the target genomic locus to create a modified target genomic locus, and the multidomain therapeutic protein comprising the CD63-binding delivery domain fused to the lysosomal alpha-glucosidase is expressed from the modified target genomic locus. In some such methods, the expressed multidomain therapeutic protein is delivered to and internalized by skeletal muscle and heart tissue in the subject. In some such methods, the cell is a liver cell or the population of cells is a population of liver cells. In some such methods, the cell is a hepatocyte or the population of cells is a population of hepatocytes. In some such methods, the cell is a human cell or the population of cells is a population of human cells. In some such methods, the cell is a neonatal cell or the population of cells is a population of neonatal cells. In some such methods, the neonatal cell or the population of neonatal cells is from a human neonatal subject within 24 weeks after birth. In some such methods, the neonatal cell or the population of neonatal cells is from a human neonatal subject within 12 weeks after birth. In some such methods, the neonatal cell or the population of neonatal cells is from a human neonatal subject within 8 weeks after birth. In some such methods, the neonatal cell or the population of neonatal cells is from a human neonatal subject within 4 weeks after birth. In some such methods, the cell is not a neonatal cell or the population of cells is not a population of neonatal cells.

In another aspect, provided are methods of treating a lysosomal alpha-glucosidase deficiency in a subject in need thereof. Some such methods comprise administering to the subject any of the above compositions, wherein the coding sequence for the multidomain therapeutic protein is operably linked to a promoter in the nucleic acid construct and is expressed in the subject, optionally wherein the delivery domain is a CD63-binding delivery domain or a TfR-binding delivery domain. Some such methods comprise administering to the subject any of the above compositions, wherein the nuclease agent cleaves the nuclease target site in the target genomic locus, the nucleic acid construct is inserted into the target genomic locus to create a modified target genomic locus, and the multidomain therapeutic protein comprising the delivery domain fused to the lysosomal alpha-glucosidase is expressed from the modified target genomic locus, optionally wherein the delivery domain is a CD63-binding delivery domain or a TfR-binding delivery domain. Some such methods comprise administering to the subject any of the above compositions, wherein the coding sequence for the multidomain therapeutic protein is operably linked to a promoter in the nucleic acid construct and the multidomain therapeutic protein comprising the CD63-binding delivery domain fused to the lysosomal alpha-glucosidase is expressed in the subject. Some such methods comprise administering to the subject any of the above compositions, wherein the nuclease agent cleaves the nuclease target site in the target genomic locus, the nucleic acid construct is inserted into the target genomic locus to create a modified target genomic locus, and the multidomain therapeutic protein comprising the CD63-binding delivery domain fused to the lysosomal alpha-glucosidase is expressed from the modified target genomic locus. In another aspect, provided are methods of reducing glycogen accumulation in a tissue in a subject in need thereof. Some such methods comprise administering to the subject any of the above compositions, wherein the coding sequence for the multidomain therapeutic protein is operably linked to a promoter in the nucleic acid construct and is expressed in the subject and reduces glycogen accumulation in the tissue, optionally wherein the delivery domain is a CD63-binding delivery domain or a TfR-binding delivery domain. Some such methods comprise administering to the subject any of the above compositions, wherein the nuclease agent cleaves the nuclease target site in the target genomic locus, the nucleic acid construct is inserted into the target genomic locus to create a modified target genomic locus, and the multidomain therapeutic protein comprising the delivery domain fused to the lysosomal alpha-glucosidase is expressed from the modified target genomic locus and reduces glycogen accumulation in the tissue, optionally wherein the delivery domain is a CD63-binding delivery domain or a TfR-binding delivery domain. Some such methods comprise administering to the subject any of the above compositions, wherein the coding sequence for the multidomain therapeutic protein is operably linked to a promoter in the nucleic acid construct and the multidomain therapeutic protein comprising the CD63-binding delivery domain fused to the lysosomal alpha-glucosidase is expressed in the subject and reduces glycogen accumulation in the tissue, optionally wherein the delivery domain is a CD63-binding delivery domain or a TfR-binding delivery domain. Some such methods comprise administering to the subject any of the above compositions, wherein the nuclease agent cleaves the nuclease target site in the target genomic locus, the nucleic acid construct is inserted into the target genomic locus to create a modified target genomic locus, and the multidomain therapeutic protein comprising the CD63-binding delivery domain fused to the lysosomal alpha-glucosidase is expressed from the modified target genomic locus and reduces glycogen accumulation in the tissue. In some such methods, the subject has Pompe disease. In another aspect, provided are methods of treating Pompe disease in a subject in need thereof. Some such methods comprise administering to the subject any of the above compositions, wherein the coding sequence for the multidomain therapeutic protein is operably linked to a promoter in the nucleic acid construct and is expressed in the subject, thereby treating the Pompe disease, optionally wherein the delivery domain is a CD63-binding delivery domain or a TfR-binding delivery domain. Some such methods comprise administering to the subject any of the above compositions, wherein the nuclease agent cleaves the nuclease target site in the target genomic locus, the nucleic acid construct is inserted into the target genomic locus to create a modified target genomic locus, and the multidomain therapeutic protein comprising the delivery domain fused to the lysosomal alpha-glucosidase is expressed from the modified target genomic locus, thereby treating the Pompe disease, optionally wherein the delivery domain is a CD63-binding delivery domain or a TfR-binding delivery domain. Some such methods comprise administering to the subject any of the above compositions, wherein the coding sequence for the multidomain therapeutic protein is operably linked to a promoter in the nucleic acid construct and the multidomain therapeutic protein comprising the CD63-binding delivery domain fused to the lysosomal alpha-glucosidase is expressed in the subject, thereby treating the Pompe disease. Some such methods comprise administering to the subject any of the above compositions, wherein the nuclease agent cleaves the nuclease target site in the target genomic locus, the nucleic acid construct is inserted into the target genomic locus to create a modified target genomic locus, and the multidomain therapeutic protein comprising the CD63-binding delivery domain fused to the lysosomal alpha-glucosidase is expressed from the modified target genomic locus, thereby treating the Pompe disease. In some such methods, the Pompe disease is infantile-onset Pompe disease. In some such methods, the Pompe disease is late-onset Pompe disease.

In some such methods, the subject is a neonatal subject. In some such methods, the neonatal subject is a human subject within 24 weeks after birth. In some such methods, the neonatal subject is a human subject within 12 weeks after birth. In some such methods, the neonatal subject is a human subject within 8 weeks after birth. In some such methods, the neonatal subject is a human subject within 4 weeks after birth. In some such methods, the subject is not a neonatal subject. In some such methods, the method results in a therapeutically effective level of circulating multidomain therapeutic protein or lysosomal alpha-glucosidase in the subject. In some such methods, the method reduces glycogen accumulation in skeletal muscle, heart tissue, or central nervous system tissue in the subject. In some such methods, the method reduces glycogen accumulation in skeletal muscle and heart tissue in the subject. In some such methods, the method results in reduced glycogen levels in skeletal muscle and/or heart tissue in the subject comparable to wild type levels at the same age. In some such methods, the method improves muscle strength in the subject or prevents loss of muscle strength in the subject compared to a control subject. In some such methods, the method results in the subject having muscle strength comparable to wild type levels at the same age.

In another aspect, provided are methods of preventing or reducing the onset of a sign or symptom of Pompe disease in a subject in need thereof. Some such methods comprise administering to the subject any of the above compositions, wherein the coding sequence for the multidomain therapeutic protein is operably linked to a promoter in the nucleic acid construct and is expressed in the subject, thereby preventing or reducing the onset of a sign or symptom of the Pompe disease in the subject, optionally wherein the delivery domain is a CD63-binding delivery domain or a TfR-binding delivery domain. Some such methods comprise administering to the subject any of the above compositions, wherein the nuclease agent cleaves the nuclease target site, the nucleic acid construct is inserted into the target genomic locus to create a modified target genomic locus, and the multidomain therapeutic protein comprising the delivery domain fused to the lysosomal alpha-glucosidase is expressed from the modified target genomic locus, thereby preventing or reducing the onset of a sign or symptom of the Pompe disease in the subject, optionally wherein the delivery domain is a CD63-binding delivery domain or a TfR-binding delivery domain. Some such methods comprise administering to the subject any of the above compositions, wherein the coding sequence for the multidomain therapeutic protein is operably linked to a promoter in the nucleic acid construct and the multidomain therapeutic protein comprising the CD63-binding delivery domain fused to the lysosomal alpha-glucosidase is expressed in the subject, thereby preventing or reducing the onset of a sign or symptom of the Pompe disease in the subject. Some such methods comprise administering to the subject any of the above compositions, wherein the nuclease agent cleaves the nuclease target site, the nucleic acid construct is inserted into the target genomic locus to create a modified target genomic locus, and the multidomain therapeutic protein comprising the CD63-binding delivery domain fused to the lysosomal alpha-glucosidase is expressed from the modified target genomic locus, thereby preventing or reducing the onset of a sign or symptom of the Pompe disease in the subject. In some such methods, the Pompe disease is infantile-onset Pompe disease. In some such methods, the Pompe disease is late-onset Pompe disease. In some such methods, the method results in a therapeutically effective level of circulating multidomain therapeutic protein or lysosomal alpha-glucosidase in the subject. In some such methods, the method prevents or reduces glycogen accumulation in skeletal muscle, heart, or central nervous system tissue in the subject. In some such methods, the method prevents or reduces glycogen accumulation in skeletal muscle, heart, and central nervous system tissue in the subject. In some such methods, the subject is a neonatal subject. In some such methods, the neonatal subject is a human subject within 24 weeks after birth. In some such methods, the neonatal subject is a human subject within 12 weeks after birth. In some such methods, the neonatal subject is a human subject within 8 weeks after birth. In some such methods, the neonatal subject is a human subject within 4 weeks after birth. In some such methods, the subject is not a neonatal subject.

In some such methods, the method results in increased expression of the multidomain therapeutic protein in the subject compared to a method comprising administering an episomal expression vector encoding the multidomain therapeutic protein to a control subject. In some such methods, the method results in increased serum levels of the multidomain therapeutic protein in the subject compared to a method comprising administering an episomal expression vector encoding the multidomain therapeutic protein to a control subject. In some such methods, the method results in serum levels of the multidomain therapeutic protein in the subject of at least about 1 µg/mL, at least about 2 µg/mL, at least about 3 µg/mL, at least about 4 µg/mL, at least about 5 µg/mL, at least about 6 µg/mL, at least about 7 µg/mL, at least about 8 µg/mL, at least about 9 µg/mL, or at least about 10 µg/mL. In some such methods, the method results in serum levels of the multidomain therapeutic protein in the subject of at least about 2 µg/mL or at least about 5 µg/mL. In some such methods, the method results in serum levels of the multidomain therapeutic protein in the subject of between about 2 µg/mL and about 30 µg/mL or between about 2 µg/mL and about 20 µg/mL. In some such methods, the method results in serum levels of the multidomain therapeutic protein in the subject of between about 5 µg/mL and about 30 µg/mL or between about 5 µg/mL and about 20 µg/mL. In some such methods, the method achieves lysosomal alpha-glucosidase activity levels of at least about 40% of normal, at least about 45% of normal, at least about 50%, at least about 60%, at least about 70%, at least about 80%, at least about 90%, or 100% of normal. In some such methods, the subject has infantile-onset Pompe disease, and the method achieves lysosomal alpha-glucosidase expression or activity levels of at least about 1% or more than about 1% of normal. In some such methods, the subject has late-onset Pompe disease, and the method achieves lysosomal alpha-glucosidase expression or activity levels of at least about 40% of normal or more than about 40% of normal. In some such methods, the method increases lysosomal alpha-glucosidase activity over the subject’s baseline lysosomal alpha-glucosidase activity by at least about 1%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or at least about 100%. In some such methods, the expression or activity of the multidomain therapeutic protein is at least 50% of the expression or activity of the multidomain therapeutic protein at a peak level of expression measured for the subject at six months or 24 weeks after the administering. In some such methods, the expression or activity of the multidomain therapeutic protein is at least 50% of the expression or activity of the multidomain therapeutic protein at a peak level of expression measured for the subject at one year after the administering. In some such methods, the expression or activity of the multidomain therapeutic protein is at least 60% of the expression or activity of the multidomain therapeutic protein at a peak level of expression measured for the subject at six months or 24 weeks after the administering. In some such methods, the expression or activity of the multidomain therapeutic protein is at least 50% of the expression or activity of the multidomain therapeutic protein at a peak level of expression measured for the subject at two years after the administering. In some such methods, the expression or activity of the multidomain therapeutic protein is at least 60% of the expression or activity of the multidomain therapeutic protein at a peak level of expression measured for the subject at two years after the administering. In some such methods, the expression or activity of the multidomain therapeutic protein is at least 60% of the expression or activity of the multidomain therapeutic protein at a peak level of expression measured for the subject at six months or 24 weeks after the administering.

In some such methods, the method further comprises assessing preexisting AAV immunity in the subject prior to administering the nucleic acid construct to the subject. In some such methods, the preexisting AAV immunity is preexisting AAV8 immunity. In some such methods, assessing preexisting AAV immunity comprises assessing immunogenicity using a total antibody immune assay or a neutralizing antibody assay.

In some such methods, the nucleic acid construct is administered simultaneously with the nuclease agent or the one or more nucleic acids encoding the nuclease agent. In some such methods, the nucleic acid construct is not administered simultaneously with the nuclease agent or the one or more nucleic acids encoding the nuclease agent. In some such methods, the nucleic acid construct is administered prior to the nuclease agent or the one or more nucleic acids encoding the nuclease agent. In some such methods, the nucleic acid construct is administered after the nuclease agent or the one or more nucleic acids encoding the nuclease agent.

In some such methods, the subject is a human subject. In some such methods, the subject is a neonatal subject. In some such methods, the neonatal subject is a human subject within 24 weeks after birth. In some such methods, the neonatal subject is a human subject within 12 weeks after birth. In some such methods, the neonatal subject is a human subject within 8 weeks after birth. In some such methods, the neonatal subject is a human subject within 4 weeks after birth. In some such methods, the subject is not a neonatal subject.

Nucleic acid constructs and compositions that allow insertion of a multidomain therapeutic protein (e.g., GAA fusion protein) coding sequence into a target genomic locus such as an endogenous ALB locus and/or expression of the multidomain therapeutic protein (e.g., GAA fusion protein) coding sequence are provided. The nucleic acid constructs and compositions can be used in methods of integrating or inserting a multidomain therapeutic protein (e.g., GAA fusion protein) nucleic acid into a target genomic locus in a cell or a population of cells or a subject, methods of expressing a multidomain therapeutic protein (e.g., GAA fusion protein) in a cell or a population of cells or a subject, methods of reducing glycogen accumulation in a cell or a population of cells or a subject, and methods of treating Pompe disease or GAA deficiency in a subject, and method of preventing or reducing the onset of a sign or symptom of Pompe disease in a subject such as subjects with reduced GAA activity or expression and in a subject diagnosed with Pompe disease, including neonatal cells and subjects. In some embodiments the cell, population of cells, or subject is a neonatal cell, a neonatal population of cells, or a neonatal subject

In one aspect, provided are methods of inserting a nucleic acid encoding a multidomain therapeutic protein comprising a TfR-binding delivery domain fused to a lysosomal alpha-glucosidase into a target genomic locus in a neonatal cell or a population of neonatal cells. Some such methods comprise administering to the neonatal cell or the population of neonatal cells: (a) a nucleic acid construct comprising a coding sequence for the multidomain therapeutic protein comprising the TfR-binding delivery domain fused to the lysosomal alpha-glucosidase; and (b) a nuclease agent or one or more nucleic acids encoding the nuclease agent, wherein the nuclease agent targets a nuclease target site in the target genomic locus, wherein the nuclease agent cleaves the nuclease target site, and the nucleic acid construct is inserted into the target genomic locus. In another aspect, provided are methods of expressing a multidomain therapeutic protein comprising a TfR-binding delivery domain fused to a lysosomal alpha-glucosidase in a neonatal cell or a population of neonatal cells. In another aspect, provided are methods of expressing a multidomain therapeutic protein comprising a TfR-binding delivery domain fused to a lysosomal alpha-glucosidase from a target genomic locus in a neonatal cell or a population of neonatal cells. Some such methods comprise administering to the neonatal cell or the population of neonatal cells: (a) a nucleic acid construct comprising a coding sequence for the multidomain therapeutic protein comprising the TfR-binding delivery domain fused to the lysosomal alpha-glucosidase; and (b) a nuclease agent or one or more nucleic acids encoding the nuclease agent, wherein the nuclease agent targets a nuclease target site in the target genomic locus, wherein the nuclease agent cleaves the nuclease target site, the nucleic acid construct is inserted into the target genomic locus to create a modified target genomic locus, and the multidomain therapeutic protein comprising the TfR-binding delivery domain fused to the lysosomal alpha-glucosidase is expressed from the modified target genomic locus. In some such methods, the neonatal cell is a liver cell or the population of neonatal cells is a population of liver cells. In some such methods, the neonatal cell is a hepatocyte or the population of neonatal cells is a population of hepatocytes. In some such methods, the neonatal cell is a human cell or the population of neonatal cells is a population of human cells. In some such methods, the neonatal cell or the population of neonatal cells is from a human neonatal subject within 24 weeks after birth. In some such methods, the neonatal cell or the population of neonatal cells is from a human neonatal subject within 12 weeks after birth. In some such methods, the neonatal cell or the population of neonatal cells is from a human neonatal subject within 8 weeks after birth. In some such methods, the neonatal cell or the population of neonatal cells is from a human neonatal subject within 4 weeks after birth. In some such methods, the neonatal cell is in vitro or ex vivo or the population of neonatal cells is in vitro or ex vivo. In some such methods, the neonatal cell is in vivo in a neonatal subject or the population of neonatal cells is in vivo in a neonatal subject.

In another aspect, provided are methods of inserting a nucleic acid encoding a multidomain therapeutic protein comprising a TfR-binding delivery domain fused to a lysosomal alpha-glucosidase into a target genomic locus in a neonatal cell or a population of neonatal cells in a neonatal subject. Some such methods comprise administering to the neonatal subject: (a) a nucleic acid construct comprising a coding sequence for the multidomain therapeutic protein comprising the TfR-binding delivery domain fused to the lysosomal alpha-glucosidase; and (b) a nuclease agent or one or more nucleic acids encoding the nuclease agent, wherein the nuclease agent targets a nuclease target site in the target genomic locus, wherein the nuclease agent cleaves the nuclease target site, and the nucleic acid construct is inserted into the target genomic locus. In another aspect, provided are methods of expressing a multidomain therapeutic protein comprising a TfR-binding delivery domain fused to a lysosomal alpha-glucosidase protein in a neonatal cell or a population of neonatal cells in a neonatal subject. In another aspect, provided are methods of expressing a multidomain therapeutic protein comprising a TfR-binding delivery domain fused to a lysosomal alpha-glucosidase protein from a target genomic locus in a neonatal cell or a population of neonatal cells in a neonatal subject. Some such methods comprise administering to the neonatal subject: (a) a nucleic acid construct comprising a coding sequence for the multidomain therapeutic protein comprising the TfR-binding delivery domain fused to the lysosomal alpha-glucosidase; and (b) a nuclease agent or one or more nucleic acids encoding the nuclease agent, wherein the nuclease agent targets a nuclease target site in a target gene at the target genomic locus, wherein the nuclease agent cleaves the nuclease target site, the nucleic acid construct is inserted into the target genomic locus to create a modified target genomic locus, and the multidomain therapeutic protein comprising the TfR-binding delivery domain fused to the lysosomal alpha-glucosidase is expressed from the modified target genomic locus. In some such methods, the expressed multidomain therapeutic protein is delivered to and internalized by skeletal muscle heart, and central nervous system tissue in the subject. In some such methods, the neonatal cell is a liver cell or the population of neonatal cells is a population of liver cells. In some such methods, the neonatal cell is a hepatocyte or the population of neonatal cells is a population of hepatocytes. In some such methods, the neonatal cell is a human cell or the population of neonatal cells is a population of human cells.

In another aspect, provided are methods of treating a lysosomal alpha-glucosidase deficiency in a neonatal subject in need thereof. Some such methods comprise administering to the neonatal subject: (a) a nucleic acid construct comprising a coding sequence for a multidomain therapeutic protein comprising a TfR-binding delivery domain fused to a lysosomal alpha-glucosidase; and (b) a nuclease agent or one or more nucleic acids encoding the nuclease agent, wherein the nuclease agent targets a nuclease target site in a target genomic locus, wherein the nuclease agent cleaves the nuclease target site, the nucleic acid construct is inserted into the target genomic locus to create a modified target genomic locus, and the multidomain therapeutic protein comprising the TfR-binding delivery domain fused to the lysosomal alpha-glucosidase is expressed from the modified target genomic locus. In another aspect, provided are methods of reducing glycogen accumulation in a tissue in a neonatal subject in need thereof. Some such methods comprise administering to the neonatal subject: (a) a nucleic acid construct comprising a coding sequence for a multidomain therapeutic protein comprising a TfR-binding delivery domain fused to a lysosomal alpha-glucosidase; and (b) a nuclease agent or one or more nucleic acids encoding the nuclease agent, wherein the nuclease agent targets a nuclease target site in a target genomic locus, wherein the nuclease agent cleaves the nuclease target site, the nucleic acid construct is inserted into the target genomic locus to create a modified target genomic locus, and the multidomain therapeutic protein comprising the TfR-binding delivery domain fused to the lysosomal alpha-glucosidase is expressed from the modified target genomic locus and reduces glycogen accumulation in the tissue. In some such methods, the subject has Pompe disease. In another aspect, provided are methods of treating Pompe disease in a neonatal subject in need thereof. Some such methods comprise administering to the neonatal subject: (a) a nucleic acid construct comprising a coding sequence for a multidomain therapeutic protein comprising a TfR-binding delivery domain fused to a lysosomal alpha-glucosidase; and (b) a nuclease agent or one or more nucleic acids encoding the nuclease agent, wherein the nuclease agent targets a nuclease target site in a target genomic locus, wherein the nuclease agent cleaves the nuclease target site, the nucleic acid construct is inserted into the target genomic locus to create a modified target genomic locus, and the multidomain therapeutic protein comprising the TfR-binding delivery domain fused to the lysosomal alpha-glucosidase is expressed from the modified target genomic locus, thereby treating the Pompe disease. In some such methods, the Pompe disease is infantile-onset Pompe disease.

In some such methods, the method results in a therapeutically effective level of circulating multidomain therapeutic protein or lysosomal alpha-glucosidase in the subject. In some such methods, the method reduces glycogen accumulation in skeletal muscle, heart, or central nervous system tissue in the subject. In some such methods, the method reduces glycogen accumulation in skeletal muscle, heart, and central nervous system tissue in the subject. In some such methods, the method results in reduced glycogen levels in skeletal muscle, heart, and/or central nervous system tissue in the subject comparable to wild type levels at the same age. In some such methods, the method improves muscle strength in the subject or prevents loss of muscle strength in the subject compared to a control subject. In some such methods, the method results in the subject having muscle strength comparable to wild type levels at the same age.

In another aspect, provided are methods of preventing or reducing the onset of a sign or symptom of Pompe disease in a neonatal subject in need thereof. Some such methods comprise administering to the neonatal subject: (a) a nucleic acid construct comprising a coding sequence for a multidomain therapeutic protein comprising a TfR-binding delivery domain fused to a lysosomal alpha-glucosidase; and (b) a nuclease agent or one or more nucleic acids encoding the nuclease agent, wherein the nuclease agent targets a nuclease target site in a target genomic locus, wherein the nuclease agent cleaves the nuclease target site, the nucleic acid construct is inserted into the target genomic locus to create a modified target genomic locus, and the multidomain therapeutic protein comprising the TfR-binding delivery domain fused to the lysosomal alpha-glucosidase is expressed from the modified target genomic locus, thereby preventing or reducing the onset of a sign or symptom of the Pompe disease in the subject. In some such methods, the Pompe disease is infantile-onset Pompe disease. In some such methods, the Pompe disease is late-onset Pompe disease. In some such methods, the method results in a therapeutically effective level of circulating multidomain therapeutic protein or lysosomal alpha-glucosidase in the subject. In some such methods, the method prevents or reduces glycogen accumulation in skeletal muscle, heart, or central nervous system tissue in the subject. In some such methods, the method prevents or reduces glycogen accumulation in skeletal muscle, heart, and central nervous system tissue in the subject. In some such methods, the neonatal subject is a human subject within 24 weeks after birth. In some such methods, the neonatal subject is a human subject within 12 weeks after birth. In some such methods, the neonatal subject is a human subject within 8 weeks after birth. In some such methods, the neonatal subject is a human subject within 4 weeks after birth.

In some such methods, the method results in increased expression of the multidomain therapeutic protein in the subject compared to a method comprising administering an episomal expression vector encoding the multidomain therapeutic protein to a control subject. In some such methods, the method results in increased serum levels of the multidomain therapeutic protein in the subject compared to a method comprising administering an episomal expression vector encoding the multidomain therapeutic protein to a control subject. In some such methods, the method results in serum levels of the multidomain therapeutic protein in the subject of at least about 1 µg/mL, at least about 2 µg/mL, at least about 3 µg/mL, at least about 4 µg/mL, at least about 5 µg/mL, at least about 6 µg/mL, at least about 7 µg/mL, at least about 8 µg/mL, at least about 9 µg/mL, or at least about 10 µg/mL. In some such methods, the method results in serum levels of the multidomain therapeutic protein in the subject of at least about 2 µg/mL or at least about 5 µg/mL. In some such methods, the method results in serum levels of the multidomain therapeutic protein in the subject of between about 2 µg/mL and about 30 µg/mL or between about 2 µg/mL and about 20 µg/mL. In some such methods, the method results in serum levels of the multidomain therapeutic protein in the subject of between about 5 µg/mL and about 30 µg/mL or between about 5 µg/mL and about 20 µg/mL. In some such methods, the method achieves lysosomal alpha-glucosidase activity levels of at least about 40% of normal, at least about 45% of normal, at least about 50%, at least about 60%, at least about 70%, at least about 80%, at least about 90%, or 100% of normal. In some such methods, the subject has infantile-onset Pompe disease, and the method achieves lysosomal alpha-glucosidase expression or activity levels of at least about 1% or more than about 1% of normal. In some such methods, the subject has late-onset Pompe disease, and the method achieves lysosomal alpha-glucosidase expression or activity levels of at least about 40% of normal or more than about 40% of normal. In some such methods, the method increases lysosomal alpha-glucosidase activity over the subject’s baseline lysosomal alpha-glucosidase activity by at least about 1%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or at least about 100%. In some such methods, the expression or activity of the multidomain therapeutic protein is at least 50% of the expression or activity of the multidomain therapeutic protein at a peak level of expression measured for the subject at six months or 24 weeks after the administering. In some such methods, the expression or activity of the multidomain therapeutic protein is at least 50% of the expression or activity of the multidomain therapeutic protein at a peak level of expression measured for the subject at one year after the administering. In some such methods, the expression or activity of the multidomain therapeutic protein is at least 60% of the expression or activity of the multidomain therapeutic protein at a peak level of expression measured for the subject at six months or 24 weeks after the administering. In some such methods, the expression or activity of the multidomain therapeutic protein is at least 50% of the expression or activity of the multidomain therapeutic protein at a peak level of expression measured for the subject at two years after the administering. In some such methods, the expression or activity of the multidomain therapeutic protein is at least 60% of the expression or activity of the multidomain therapeutic protein at a peak level of expression measured for the subject at two years after the administering. In some such methods, the expression or activity of the multidomain therapeutic protein is at least 60% of the expression or activity of the multidomain therapeutic protein at a peak level of expression measured for the subject at six months or 24 weeks after the administering.

In some such methods, the method further comprises assessing preexisting AAV immunity in the neonatal subject prior to administering the nucleic acid construct to the subject. In some such methods, the preexisting AAV immunity is preexisting AAV8 immunity. In some such methods, assessing preexisting AAV immunity comprises assessing immunogenicity using a total antibody immune assay or a neutralizing antibody assay.

In some such methods, the nucleic acid construct is administered simultaneously with the nuclease agent or the one or more nucleic acids encoding the nuclease agent. In some such methods, the nucleic acid construct is not administered simultaneously with the nuclease agent or the one or more nucleic acids encoding the nuclease agent. In some such methods, the nucleic acid construct is administered prior to the nuclease agent or the one or more nucleic acids encoding the nuclease agent. In some such methods, the nucleic acid construct is administered after the nuclease agent or the one or more nucleic acids encoding the nuclease agent.

In some such methods, the TfR-binding delivery domain is fused to the lysosomal alpha-glucosidase protein via a peptide linker. In some such methods, the coding sequence for the TfR-binding delivery domain is codon-optimized or CpG-depleted. In some such methods, the coding sequence for the TfR-binding delivery domain is codon-optimized and CpG-depleted. In some such methods, the TfR-binding delivery domain comprises an anti-TfR antigen-binding protein. In some such methods, the anti-TfR antigen binding protein comprises: (i) a HCVR that comprises the HCDR1, HCDR2 and HCDR3 of a HCVR comprising the amino acid sequence set forth in SEQ ID NO: 217, 227, 237, 247, 257, 267, 277, 287, 297, 307, 317, 327, 337, 347, 357, 367, 377, 387, 397, 407, 417, 427, 437, 447, 457, 467, 477, 487, 497, 507, 517, or 527 (or a variant thereof); and/or (ii) a LCVR that comprises the LCDR1, LCDR2 and LCDR3 of a LCVR comprising the amino acid sequence set forth in SEQ ID NO: 222, 232, 242, 252, 262, 272, 282, 292, 302, 312, 322, 332, 342, 352, 362, 372, 382, 392, 402, 412, 422, 432, 442, 452, 462, 472, 482, 492, 502, 512, 522, or 532 (or a variant thereof). In some such methods, the anti-TfR antigen binding protein comprises: (1) a HCVR comprising the HCDR1, HCDR2 and HCDR3 of a HCVR that comprises the amino acid sequence set forth in SEQ ID NO: 217 (or a variant thereof); and a LCVR comprising the LCDR1, LCDR2 and LCDR3 of a LCVR that comprises the amino acid sequence set forth in SEQ ID NO: 222 (or a variant thereof); (2) a HCVR comprising the HCDR1, HCDR2 and HCDR3 of a HCVR that comprises the amino acid sequence set forth in SEQ ID NO: 227 (or a variant thereof); and a LCVR comprising the LCDR1, LCDR2 and LCDR3 of a LCVR that comprises the amino acid sequence set forth in SEQ ID NO: 232 (or a variant thereof); (3) a HCVR comprising the HCDR1, HCDR2 and HCDR3 of a HCVR that comprises the amino acid sequence set forth in SEQ ID NO: 237 (or a variant thereof); and a LCVR comprising the LCDR1, LCDR2 and LCDR3 of a LCVR that comprises the amino acid sequence set forth in SEQ ID NO: 242 (or a variant thereof); (4) a HCVR comprising the HCDR1, HCDR2 and HCDR3 of a HCVR that comprises the amino acid sequence set forth in SEQ ID NO: 247 (or a variant thereof); and a LCVR comprising the LCDR1, LCDR2 and LCDR3 of a LCVR that comprises the amino acid sequence set forth in SEQ ID NO: 252 (or a variant thereof); (5) a HCVR comprising the HCDR1, HCDR2 and HCDR3 of a HCVR that comprises the amino acid sequence set forth in SEQ ID NO: 257 (or a variant thereof); and a LCVR comprising the LCDR1, LCDR2 and LCDR3 of a LCVR that comprises the amino acid sequence set forth in SEQ ID NO: 262 (or a variant thereof); (6) a HCVR comprising the HCDR1, HCDR2 and HCDR3 of a HCVR that comprises the amino acid sequence set forth in SEQ ID NO: 267 (or a variant thereof); and a LCVR comprising the LCDR1, LCDR2 and LCDR3 of a LCVR that comprises the amino acid sequence set forth in SEQ ID NO: 272 (or a variant thereof); (7) a HCVR comprising the HCDR1, HCDR2 and HCDR3 of a HCVR that comprises the amino acid sequence set forth in SEQ ID NO: 277 (or a variant thereof); and a LCVR comprising the LCDR1, LCDR2 and LCDR3 of a LCVR that comprises the amino acid sequence set forth in SEQ ID NO: 282 (or a variant thereof); (8) a HCVR comprising the HCDR1, HCDR2 and HCDR3 of a HCVR that comprises the amino acid sequence set forth in SEQ ID NO: 287 (or a variant thereof); and a LCVR comprising the LCDR1, LCDR2 and LCDR3 of a LCVR that comprises the amino acid sequence set forth in SEQ ID NO: 292 (or a variant thereof); (9) a HCVR comprising the HCDR1, HCDR2 and HCDR3 of a HCVR that comprises the amino acid sequence set forth in SEQ ID NO: 297 (or a variant thereof); and a LCVR comprising the LCDR1, LCDR2 and LCDR3 of a LCVR that comprises the amino acid sequence set forth in SEQ ID NO: 302 (or a variant thereof); (10) a HCVR comprising the HCDR1, HCDR2 and HCDR3 of a HCVR that comprises the amino acid sequence set forth in SEQ ID NO: 307 (or a variant thereof); and a LCVR comprising the LCDR1, LCDR2 and LCDR3 of a LCVR that comprises the amino acid sequence set forth in SEQ ID NO: 312 (or a variant thereof); (11) a HCVR comprising the HCDR1, HCDR2 and HCDR3 of a HCVR that comprises the amino acid sequence set forth in SEQ ID NO: 317 (or a variant thereof); and a LCVR comprising the LCDR1, LCDR2 and LCDR3 of a LCVR that comprises the amino acid sequence set forth in SEQ ID NO: 322 (or a variant thereof); (12) a HCVR comprising the HCDR1, HCDR2 and HCDR3 of a HCVR that comprises the amino acid sequence set forth in SEQ ID NO: 327 (or a variant thereof); and a LCVR comprising the LCDR1, LCDR2 and LCDR3 of a LCVR that comprises the amino acid sequence set forth in SEQ ID NO: 332 (or a variant thereof); (13) a HCVR comprising the HCDR1, HCDR2 and HCDR3 of a HCVR that comprises the amino acid sequence set forth in SEQ ID NO: 337 (or a variant thereof); and a LCVR comprising the LCDR1, LCDR2 and LCDR3 of a LCVR that comprises the amino acid sequence set forth in SEQ ID NO: 342 (or a variant thereof); (14) a HCVR comprising the HCDR1, HCDR2 and HCDR3 of a HCVR that comprises the amino acid sequence set forth in SEQ ID NO: 347 (or a variant thereof); and a LCVR comprising the LCDR1, LCDR2 and LCDR3 of a LCVR that comprises the amino acid sequence set forth in SEQ ID NO: 352 (or a variant thereof); (15) a HCVR comprising the HCDR1, HCDR2 and HCDR3 of a HCVR that comprises the amino acid sequence set forth in SEQ ID NO: 357 (or a variant thereof); and a LCVR comprising the LCDR1, LCDR2 and LCDR3 of a LCVR that comprises the amino acid sequence set forth in SEQ ID NO: 362 (or a variant thereof); (16) a HCVR comprising the HCDR1, HCDR2 and HCDR3 of a HCVR that comprises the amino acid sequence set forth in SEQ ID NO: 367 (or a variant thereof); and a LCVR comprising the LCDR1, LCDR2 and LCDR3 of a LCVR that comprises the amino acid sequence set forth in SEQ ID NO: 372 (or a variant thereof); (17) a HCVR comprising the HCDR1, HCDR2 and HCDR3 of a HCVR that comprises the amino acid sequence set forth in SEQ ID NO: 377 (or a variant thereof); and a LCVR comprising the LCDR1, LCDR2 and LCDR3 of a LCVR that comprises the amino acid sequence set forth in SEQ ID NO: 382 (or a variant thereof); (18) a HCVR comprising the HCDR1, HCDR2 and HCDR3 of a HCVR that comprises the amino acid sequence set forth in SEQ ID NO: 387 (or a variant thereof); and a LCVR comprising the LCDR1, LCDR2 and LCDR3 of a LCVR that comprises the amino acid sequence set forth in SEQ ID NO: 392 (or a variant thereof); (19) a HCVR comprising the HCDR1, HCDR2 and HCDR3 of a HCVR that comprises the amino acid sequence set forth in SEQ ID NO: 397 (or a variant thereof); and a LCVR comprising the LCDR1, LCDR2 and LCDR3 of a LCVR that comprises the amino acid sequence set forth in SEQ ID NO: 402 (or a variant thereof); (20) a HCVR comprising the HCDR1, HCDR2 and HCDR3 of a HCVR that comprises the amino acid sequence set forth in SEQ ID NO: 407 (or a variant thereof); and a LCVR comprising the LCDR1, LCDR2 and LCDR3 of a LCVR that comprises the amino acid sequence set forth in SEQ ID NO: 412 (or a variant thereof); (21) a HCVR comprising the HCDR1, HCDR2 and HCDR3 of a HCVR that comprises the amino acid sequence set forth in SEQ ID NO: 417 (or a variant thereof); and a LCVR comprising the LCDR1, LCDR2 and LCDR3 of a LCVR that comprises the amino acid sequence set forth in SEQ ID NO: 422 (or a variant thereof); (22) a HCVR comprising the HCDR1, HCDR2 and HCDR3 of a HCVR that comprises the amino acid sequence set forth in SEQ ID NO: 427 (or a variant thereof); and a LCVR comprising the LCDR1, LCDR2 and LCDR3 of a LCVR that comprises the amino acid sequence set forth in SEQ ID NO: 432 (or a variant thereof); (23) a HCVR comprising the HCDR1, HCDR2 and HCDR3 of a HCVR that comprises the amino acid sequence set forth in SEQ ID NO: 437 (or a variant thereof); and a LCVR comprising the LCDR1, LCDR2 and LCDR3 of a LCVR that comprises the amino acid sequence set forth in SEQ ID NO: 442 (or a variant thereof); (24) a HCVR comprising the HCDR1, HCDR2 and HCDR3 of a HCVR that comprises the amino acid sequence set forth in SEQ ID NO: 447 (or a variant thereof); and a LCVR comprising the LCDR1, LCDR2 and LCDR3 of a LCVR that comprises the amino acid sequence set forth in SEQ ID NO: 452 (or a variant thereof); (25) a HCVR comprising the HCDR1, HCDR2 and HCDR3 of a HCVR that comprises the amino acid sequence set forth in SEQ ID NO: 457 (or a variant thereof); and a LCVR comprising the LCDR1, LCDR2 and LCDR3 of a LCVR that comprises the amino acid sequence set forth in SEQ ID NO: 462 (or a variant thereof); (26) a HCVR comprising the HCDR1, HCDR2 and HCDR3 of a HCVR that comprises the amino acid sequence set forth in SEQ ID NO: 467 (or a variant thereof); and a LCVR comprising the LCDR1, LCDR2 and LCDR3 of a LCVR that comprises the amino acid sequence set forth in SEQ ID NO: 472 (or a variant thereof); (27) a HCVR comprising the HCDR1, HCDR2 and HCDR3 of a HCVR that comprises the amino acid sequence set forth in SEQ ID NO: 477 (or a variant thereof); and a LCVR comprising the LCDR1, LCDR2 and LCDR3 of a LCVR that comprises the amino acid sequence set forth in SEQ ID NO: 482 (or a variant thereof); (28) a HCVR comprising the HCDR1, HCDR2 and HCDR3 of a HCVR that comprises the amino acid sequence set forth in SEQ ID NO: 487 (or a variant thereof); and a LCVR comprising the LCDR1, LCDR2 and LCDR3 of a LCVR that comprises the amino acid sequence set forth in SEQ ID NO: 492 (or a variant thereof); (29) a HCVR comprising the HCDR1, HCDR2 and HCDR3 of a HCVR that comprises the amino acid sequence set forth in SEQ ID NO: 497 (or a variant thereof); and a LCVR comprising the LCDR1, LCDR2 and LCDR3 of a LCVR that comprises the amino acid sequence set forth in SEQ ID NO: 502 (or a variant thereof); (30) a HCVR comprising the HCDR1, HCDR2 and HCDR3 of a HCVR that comprises the amino acid sequence set forth in SEQ ID NO: 507 (or a variant thereof); and a LCVR comprising the LCDR1, LCDR2 and LCDR3 of a LCVR that comprises the amino acid sequence set forth in SEQ ID NO: 512 (or a variant thereof); (31) a HCVR comprising the HCDR1, HCDR2 and HCDR3 of a HCVR that comprises the amino acid sequence set forth in SEQ ID NO: 517 (or a variant thereof); and a LCVR comprising the LCDR1, LCDR2 and LCDR3 of a LCVR that comprises the amino acid sequence set forth in SEQ ID NO: 522 (or a variant thereof); or (32) a HCVR comprising the HCDR1, HCDR2 and HCDR3 of a HCVR that comprises the amino acid sequence set forth in SEQ ID NO: 527 (or a variant thereof); and a LCVR comprising the LCDR1, LCDR2 and LCDR3 of a LCVR that comprises the amino acid sequence set forth in SEQ ID NO: 532 (or a variant thereof). In some such methods, the anti-TfR antigen binding protein comprises: (1) a HCVR comprising the HCDR1, HCDR2 and HCDR3 of a HCVR that comprises the amino acid sequence set forth in SEQ ID NO: 437 (or a variant thereof); and a LCVR comprising the LCDR1, LCDR2 and LCDR3 of a LCVR that comprises the amino acid sequence set forth in SEQ ID NO: 442 (or a variant thereof); or (2) a HCVR comprising the HCDR1, HCDR2 and HCDR3 of a HCVR that comprises the amino acid sequence set forth in SEQ ID NO: 457 (or a variant thereof); and a LCVR comprising the LCDR1, LCDR2 and LCDR3 of a LCVR that comprises the amino acid sequence set forth in SEQ ID NO: 462 (or a variant thereof). In some such methods, the anti-TfR antigen binding protein comprises: (a) a HCVR that comprises: an HCDR1 comprising the amino acid sequence set forth in SEQ ID NO: 218 (or a variant thereof), an HCDR2 comprising the amino acid sequence set forth in SEQ ID NO: 219 (or a variant thereof), and an HCDR3 comprising the amino acid sequence set forth in SEQ ID NO: 220 (or a variant thereof); and a LCVR that comprises: an LCDR1 comprising the amino acid sequence set forth in SEQ ID NO: 223 (or a variant thereof), an LCDR2 comprising the amino acid sequence set forth in SEQ ID NO: 224 (or a variant thereof), and an LCDR3 comprising the amino acid sequence set forth in SEQ ID NO: 225 (or a variant thereof); (b) a HCVR that comprises: an HCDR1 comprising the amino acid sequence set forth in SEQ ID NO: 228 (or a variant thereof), an HCDR2 comprising the amino acid sequence set forth in SEQ ID NO: 229 (or a variant thereof), and an HCDR3 comprising the amino acid sequence set forth in SEQ ID NO: 230 (or a variant thereof); and a LCVR that comprises: an LCDR1 comprising the amino acid sequence set forth in SEQ ID NO: 233 (or a variant thereof), an LCDR2 comprising the amino acid sequence set forth in SEQ ID NO: 234 (or a variant thereof), and an LCDR3 comprising the amino acid sequence set forth in SEQ ID NO: 235 (or a variant thereof); (c) a HCVR that comprises: an HCDR1 comprising the amino acid sequence set forth in SEQ ID NO: 238 (or a variant thereof), an HCDR2 comprising the amino acid sequence set forth in SEQ ID NO: 239 (or a variant thereof), and an HCDR3 comprising the amino acid sequence set forth in SEQ ID NO: 240 (or a variant thereof); and a LCVR that comprises: an LCDR1 comprising the amino acid sequence set forth in SEQ ID NO: 243 (or a variant thereof), an LCDR2 comprising the amino acid sequence set forth in SEQ ID NO: 244 (or a variant thereof), and an LCDR3 comprising the amino acid sequence set forth in SEQ ID NO: 245 (or a variant thereof); (d) a HCVR that comprises: an HCDR1 comprising the amino acid sequence set forth in SEQ ID NO: 248 (or a variant thereof), an HCDR2 comprising the amino acid sequence set forth in SEQ ID NO: 249 (or a variant thereof), and an HCDR3 comprising the amino acid sequence set forth in SEQ ID NO: 250 (or a variant thereof); and a LCVR that comprises: an LCDR1 comprising the amino acid sequence set forth in SEQ ID NO: 253 (or a variant thereof), an LCDR2 comprising the amino acid sequence set forth in SEQ ID NO: 254 (or a variant thereof), and an LCDR3 comprising the amino acid sequence set forth in SEQ ID NO: 255 (or a variant thereof); (e) a HCVR that comprises: an HCDR1 comprising the amino acid sequence set forth in SEQ ID NO: 258 (or a variant thereof), an HCDR2 comprising the amino acid sequence set forth in SEQ ID NO: 259 (or a variant thereof), and an HCDR3 comprising the amino acid sequence set forth in SEQ ID NO: 260 (or a variant thereof); and a LCVR that comprises: an LCDR1 comprising the amino acid sequence set forth in SEQ ID NO: 263 (or a variant thereof), an LCDR2 comprising the amino acid sequence set forth in SEQ ID NO: 264 (or a variant thereof), and an LCDR3 comprising the amino acid sequence set forth in SEQ ID NO: 265 (or a variant thereof); (f) a HCVR that comprises: an HCDR1 comprising the amino acid sequence set forth in SEQ ID NO: 268 (or a variant thereof), an HCDR2 comprising the amino acid sequence set forth in SEQ ID NO: 269 (or a variant thereof), and an HCDR3 comprising the amino acid sequence set forth in SEQ ID NO: 270 (or a variant thereof); and a LCVR that comprises: an LCDR1 comprising the amino acid sequence set forth in SEQ ID NO: 273 (or a variant thereof), an LCDR2 comprising the amino acid sequence set forth in SEQ ID NO: 274 (or a variant thereof), and an LCDR3 comprising the amino acid sequence set forth in SEQ ID NO: 275 (or a variant thereof); (g) a HCVR that comprises: an HCDR1 comprising the amino acid sequence set forth in SEQ ID NO: 278 (or a variant thereof), an HCDR2 comprising the amino acid sequence set forth in SEQ ID NO: 279 (or a variant thereof), and an HCDR3 comprising the amino acid sequence set forth in SEQ ID NO: 280 (or a variant thereof); and a LCVR that comprises: an LCDR1 comprising the amino acid sequence set forth in SEQ ID NO: 283 (or a variant thereof), an LCDR2 comprising the amino acid sequence set forth in SEQ ID NO: 284 (or a variant thereof), and an LCDR3 comprising the amino acid sequence set forth in SEQ ID NO: 285 (or a variant thereof); (h) a HCVR that comprises: an HCDR1 comprising the amino acid sequence set forth in SEQ ID NO: 288 (or a variant thereof), an HCDR2 comprising the amino acid sequence set forth in SEQ ID NO: 289 (or a variant thereof), and an HCDR3 comprising the amino acid sequence set forth in SEQ ID NO: 290 (or a variant thereof); and a LCVR that comprises: an LCDR1 comprising the amino acid sequence set forth in SEQ ID NO: 293 (or a variant thereof), an LCDR2 comprising the amino acid sequence set forth in SEQ ID NO: 294 (or a variant thereof), and an LCDR3 comprising the amino acid sequence set forth in SEQ ID NO: 295 (or a variant thereof); (i) a HCVR that comprises: an HCDR1 comprising the amino acid sequence set forth in SEQ ID NO: 298 (or a variant thereof), an HCDR2 comprising the amino acid sequence set forth in SEQ ID NO: 299 (or a variant thereof), and an HCDR3 comprising the amino acid sequence set forth in SEQ ID NO: 300 (or a variant thereof); and a LCVR that comprises: an LCDR1 comprising the amino acid sequence set forth in SEQ ID NO: 303 (or a variant thereof), an LCDR2 comprising the amino acid sequence set forth in SEQ ID NO: 304 (or a variant thereof), and an LCDR3 comprising the amino acid sequence set forth in SEQ ID NO: 305 (or a variant thereof); (j) a HCVR that comprises: an HCDR1 comprising the amino acid sequence set forth in SEQ ID NO: 308 (or a variant thereof), an HCDR2 comprising the amino acid sequence set forth in SEQ ID NO: 309 (or a variant thereof), and an HCDR3 comprising the amino acid sequence set forth in SEQ ID NO: 310 (or a variant thereof); and a LCVR that comprises: an LCDR1 comprising the amino acid sequence set forth in SEQ ID NO: 313 (or a variant thereof), an LCDR2 comprising the amino acid sequence set forth in SEQ ID NO: 314 (or a variant thereof), and an LCDR3 comprising the amino acid sequence set forth in SEQ ID NO: 315 (or a variant thereof); (k) a HCVR that comprises: an HCDR1 comprising the amino acid sequence set forth in SEQ ID NO: 318 (or a variant thereof), an HCDR2 comprising the amino acid sequence set forth in SEQ ID NO: 319 (or a variant thereof), and an HCDR3 comprising the amino acid sequence set forth in SEQ ID NO: 320 (or a variant thereof); and a LCVR that comprises: an LCDR1 comprising the amino acid sequence set forth in SEQ ID NO: 323 (or a variant thereof), an LCDR2 comprising the amino acid sequence set forth in SEQ ID NO: 324 (or a variant thereof), and an LCDR3 comprising the amino acid sequence set forth in SEQ ID NO: 325 (or a variant thereof); (1) a HCVR that comprises: an HCDR1 comprising the amino acid sequence set forth in SEQ ID NO: 328 (or a variant thereof), an HCDR2 comprising the amino acid sequence set forth in SEQ ID NO: 329 (or a variant thereof), and an HCDR3 comprising the amino acid sequence set forth in SEQ ID NO: 330 (or a variant thereof); and a LCVR that comprises: an LCDR1 comprising the amino acid sequence set forth in SEQ ID NO: 333 (or a variant thereof), an LCDR2 comprising the amino acid sequence set forth in SEQ ID NO: 334 (or a variant thereof), and an LCDR3 comprising the amino acid sequence set forth in SEQ ID NO: 335 (or a variant thereof); (m) a HCVR that comprises: an HCDR1 comprising the amino acid sequence set forth in SEQ ID NO: 338 (or a variant thereof), an HCDR2 comprising the amino acid sequence set forth in SEQ ID NO: 339 (or a variant thereof), and an HCDR3 comprising the amino acid sequence set forth in SEQ ID NO: 340 (or a variant thereof); and a LCVR that comprises: an LCDR1 comprising the amino acid sequence set forth in SEQ ID NO: 343 (or a variant thereof), an LCDR2 comprising the amino acid sequence set forth in SEQ ID NO: 344 (or a variant thereof), and an LCDR3 comprising the amino acid sequence set forth in SEQ ID NO: 345 (or a variant thereof); (n) a HCVR that comprises: an HCDR1 comprising the amino acid sequence set forth in SEQ ID NO: 348 (or a variant thereof), an HCDR2 comprising the amino acid sequence set forth in SEQ ID NO: 349 (or a variant thereof), and an HCDR3 comprising the amino acid sequence set forth in SEQ ID NO: 350 (or a variant thereof); and a LCVR that comprises: an LCDR1 comprising the amino acid sequence set forth in SEQ ID NO: 353 (or a variant thereof), an LCDR2 comprising the amino acid sequence set forth in SEQ ID NO: 354 (or a variant thereof), and an LCDR3 comprising the amino acid sequence set forth in SEQ ID NO: 355 (or a variant thereof); (o) a HCVR that comprises: an HCDR1 comprising the amino acid sequence set forth in SEQ ID NO: 358 (or a variant thereof), an HCDR2 comprising the amino acid sequence set forth in SEQ ID NO: 359 (or a variant thereof), and an HCDR3 comprising the amino acid sequence set forth in SEQ ID NO: 360 (or a variant thereof); and a LCVR that comprises: an LCDR1 comprising the amino acid sequence set forth in SEQ ID NO: 363 (or a variant thereof), an LCDR2 comprising the amino acid sequence set forth in SEQ ID NO: 364 (or a variant thereof), and an LCDR3 comprising the amino acid sequence set forth in SEQ ID NO: 365 (or a variant thereof); (p) a HCVR that comprises: an HCDR1 comprising the amino acid sequence set forth in SEQ ID NO: 368 (or a variant thereof), an HCDR2 comprising the amino acid sequence set forth in SEQ ID NO: 369 (or a variant thereof), and an HCDR3 comprising the amino acid sequence set forth in SEQ ID NO: 370 (or a variant thereof); and a LCVR that comprises: an LCDR1 comprising the amino acid sequence set forth in SEQ ID NO: 373 (or a variant thereof), an LCDR2 comprising the amino acid sequence set forth in SEQ ID NO: 374 (or a variant thereof), and an LCDR3 comprising the amino acid sequence set forth in SEQ ID NO: 375 (or a variant thereof); (q) a HCVR that comprises: an HCDR1 comprising the amino acid sequence set forth in SEQ ID NO: 378 (or a variant thereof), an HCDR2 comprising the amino acid sequence set forth in SEQ ID NO: 379 (or a variant thereof), and an HCDR3 comprising the amino acid sequence set forth in SEQ ID NO: 380 (or a variant thereof); and a LCVR that comprises: an LCDR1 comprising the amino acid sequence set forth in SEQ ID NO: 383 (or a variant thereof), an LCDR2 comprising the amino acid sequence set forth in SEQ ID NO: 384 (or a variant thereof), and an LCDR3 comprising the amino acid sequence set forth in SEQ ID NO: 385 (or a variant thereof); (r) a HCVR that comprises: an HCDR1 comprising the amino acid sequence set forth in SEQ ID NO: 388 (or a variant thereof), an HCDR2 comprising the amino acid sequence set forth in SEQ ID NO: 389 (or a variant thereof), and an HCDR3 comprising the amino acid sequence set forth in SEQ ID NO: 390 (or a variant thereof); and a LCVR that comprises: an LCDR1 comprising the amino acid sequence set forth in SEQ ID NO: 393 (or a variant thereof), an LCDR2 comprising the amino acid sequence set forth in SEQ ID NO: 394 (or a variant thereof), and an LCDR3 comprising the amino acid sequence set forth in SEQ ID NO: 395 (or a variant thereof); (s) a HCVR that comprises: an HCDR1 comprising the amino acid sequence set forth in SEQ ID NO: 398 (or a variant thereof), an HCDR2 comprising the amino acid sequence set forth in SEQ ID NO: 399 (or a variant thereof), and an HCDR3 comprising the amino acid sequence set forth in SEQ ID NO: 400 (or a variant thereof); and a LCVR that comprises: an LCDR1 comprising the amino acid sequence set forth in SEQ ID NO: 403 (or a variant thereof), an LCDR2 comprising the amino acid sequence set forth in SEQ ID NO: 404 (or a variant thereof), and an LCDR3 comprising the amino acid sequence set forth in SEQ ID NO: 405 (or a variant thereof); (t) a HCVR that comprises: an HCDR1 comprising the amino acid sequence set forth in SEQ ID NO: 408 (or a variant thereof), an HCDR2 comprising the amino acid sequence set forth in SEQ ID NO: 409 (or a variant thereof), and an HCDR3 comprising the amino acid sequence set forth in SEQ ID NO: 410 (or a variant thereof); and a LCVR that comprises: an LCDR1 comprising the amino acid sequence set forth in SEQ ID NO: 413 (or a variant thereof), an LCDR2 comprising the amino acid sequence set forth in SEQ ID NO: 414 (or a variant thereof), and an LCDR3 comprising the amino acid sequence set forth in SEQ ID NO: 415 (or a variant thereof); (u) a HCVR that comprises: an HCDR1 comprising the amino acid sequence set forth in SEQ ID NO: 418 (or a variant thereof), an HCDR2 comprising the amino acid sequence set forth in SEQ ID NO: 419 (or a variant thereof), and an HCDR3 comprising the amino acid sequence set forth in SEQ ID NO: 420 (or a variant thereof); and a LCVR that comprises: an LCDR1 comprising the amino acid sequence set forth in SEQ ID NO: 423 (or a variant thereof), an LCDR2 comprising the amino acid sequence set forth in SEQ ID NO: 424 (or a variant thereof), and an LCDR3 comprising the amino acid sequence set forth in SEQ ID NO: 425 (or a variant thereof); (v) a HCVR that comprises: an HCDR1 comprising the amino acid sequence set forth in SEQ ID NO: 428 (or a variant thereof), an HCDR2 comprising the amino acid sequence set forth in SEQ ID NO: 429 (or a variant thereof), and an HCDR3 comprising the amino acid sequence set forth in SEQ ID NO: 430 (or a variant thereof); and a LCVR that comprises: an LCDR1 comprising the amino acid sequence set forth in SEQ ID NO: 433 (or a variant thereof), an LCDR2 comprising the amino acid sequence set forth in SEQ ID NO: 434 (or a variant thereof), and an LCDR3 comprising the amino acid sequence set forth in SEQ ID NO: 435 (or a variant thereof); (w) a HCVR that comprises: an HCDR1 comprising the amino acid sequence set forth in SEQ ID NO: 438 (or a variant thereof), an HCDR2 comprising the amino acid sequence set forth in SEQ ID NO: 439 (or a variant thereof), and an HCDR3 comprising the amino acid sequence set forth in SEQ ID NO: 440 (or a variant thereof); and a LCVR that comprises: an LCDR1 comprising the amino acid sequence set forth in SEQ ID NO: 443 (or a variant thereof), an LCDR2 comprising the amino acid sequence set forth in SEQ ID NO: 444 (or a variant thereof), and an LCDR3 comprising the amino acid sequence set forth in SEQ ID NO: 445 (or a variant thereof); (x) a HCVR that comprises: an HCDR1 comprising the amino acid sequence set forth in SEQ ID NO: 448 (or a variant thereof), an HCDR2 comprising the amino acid sequence set forth in SEQ ID NO: 449 (or a variant thereof), and an HCDR3 comprising the amino acid sequence set forth in SEQ ID NO: 450 (or a variant thereof); and a LCVR that comprises: an LCDR1 comprising the amino acid sequence set forth in SEQ ID NO: 453 (or a variant thereof), an LCDR2 comprising the amino acid sequence set forth in SEQ ID NO: 454 (or a variant thereof), and an LCDR3 comprising the amino acid sequence set forth in SEQ ID NO: 455 (or a variant thereof); (y) a HCVR that comprises: an HCDR1 comprising the amino acid sequence set forth in SEQ ID NO: 458 (or a variant thereof), an HCDR2 comprising the amino acid sequence set forth in SEQ ID NO: 459 (or a variant thereof), and an HCDR3 comprising the amino acid sequence set forth in SEQ ID NO: 460 (or a variant thereof); and a LCVR that comprises: an LCDR1 comprising the amino acid sequence set forth in SEQ ID NO: 463 (or a variant thereof), an LCDR2 comprising the amino acid sequence set forth in SEQ ID NO: 464 (or a variant thereof), and an LCDR3 comprising the amino acid sequence set forth in SEQ ID NO: 465 (or a variant thereof); (z) a HCVR that comprises: an HCDR1 comprising the amino acid sequence set forth in SEQ ID NO: 468 (or a variant thereof), an HCDR2 comprising the amino acid sequence set forth in SEQ ID NO: 469 (or a variant thereof), and an HCDR3 comprising the amino acid sequence set forth in SEQ ID NO: 470 (or a variant thereof); and a LCVR that comprises: an LCDR1 comprising the amino acid sequence set forth in SEQ ID NO: 473 (or a variant thereof), an LCDR2 comprising the amino acid sequence set forth in SEQ ID NO: 474 (or a variant thereof), and an LCDR3 comprising the amino acid sequence set forth in SEQ ID NO: 475 (or a variant thereof); (aa) a HCVR that comprises: an HCDR1 comprising the amino acid sequence set forth in SEQ ID NO: 478 (or a variant thereof), an HCDR2 comprising the amino acid sequence set forth in SEQ ID NO: 479 (or a variant thereof), and an HCDR3 comprising the amino acid sequence set forth in SEQ ID NO: 480 (or a variant thereof); and a LCVR that comprises: an LCDR1 comprising the amino acid sequence set forth in SEQ ID NO: 483 (or a variant thereof), an LCDR2 comprising the amino acid sequence set forth in SEQ ID NO: 484 (or a variant thereof), and an LCDR3 comprising the amino acid sequence set forth in SEQ ID NO: 485 (or a variant thereof); (ab) a HCVR that comprises: an HCDR1 comprising the amino acid sequence set forth in SEQ ID NO: 488 (or a variant thereof), an HCDR2 comprising the amino acid sequence set forth in SEQ ID NO: 489 (or a variant thereof), and an HCDR3 comprising the amino acid sequence set forth in SEQ ID NO: 490 (or a variant thereof); and a LCVR that comprises: an LCDR1 comprising the amino acid sequence set forth in SEQ ID NO: 493 (or a variant thereof), an LCDR2 comprising the amino acid sequence set forth in SEQ ID NO: 494 (or a variant thereof), and an LCDR3 comprising the amino acid sequence set forth in SEQ ID NO: 495 (or a variant thereof); (ac) a HCVR that comprises: an HCDR1 comprising the amino acid sequence set forth in SEQ ID NO: 498 (or a variant thereof), an HCDR2 comprising the amino acid sequence set forth in SEQ ID NO: 499 (or a variant thereof), and an HCDR3 comprising the amino acid sequence set forth in SEQ ID NO: 500 (or a variant thereof); and a LCVR that comprises: an LCDR1 comprising the amino acid sequence set forth in SEQ ID NO: 503 (or a variant thereof), an LCDR2 comprising the amino acid sequence set forth in SEQ ID NO: 504 (or a variant thereof), and an LCDR3 comprising the amino acid sequence set forth in SEQ ID NO: 505 (or a variant thereof); (ad) a HCVR that comprises: an HCDR1 comprising the amino acid sequence set forth in SEQ ID NO: 508 (or a variant thereof), an HCDR2 comprising the amino acid sequence set forth in SEQ ID NO: 509 (or a variant thereof), and an HCDR3 comprising the amino acid sequence set forth in SEQ ID NO: 510 (or a variant thereof); and a LCVR that comprises: an LCDR1 comprising the amino acid sequence set forth in SEQ ID NO: 513 (or a variant thereof), an LCDR2 comprising the amino acid sequence set forth in SEQ ID NO: 514 (or a variant thereof), and an LCDR3 comprising the amino acid sequence set forth in SEQ ID NO: 515 (or a variant thereof); (ae) a HCVR that comprises: an HCDR1 comprising the amino acid sequence set forth in SEQ ID NO: 518 (or a variant thereof), an HCDR2 comprising the amino acid sequence set forth in SEQ ID NO: 519 (or a variant thereof), and an HCDR3 comprising the amino acid sequence set forth in SEQ ID NO: 520 (or a variant thereof); and a LCVR that comprises: an LCDR1 comprising the amino acid sequence set forth in SEQ ID NO: 523 (or a variant thereof), an LCDR2 comprising the amino acid sequence set forth in SEQ ID NO: 524 (or a variant thereof), and an LCDR3 comprising the amino acid sequence set forth in SEQ ID NO: 525 (or a variant thereof); and/or (af) a HCVR that comprises: an HCDR1 comprising the amino acid sequence set forth in SEQ ID NO: 528 (or a variant thereof), an HCDR2 comprising the amino acid sequence set forth in SEQ ID NO: 529 (or a variant thereof), and an HCDR3 comprising the amino acid sequence set forth in SEQ ID NO: 530 (or a variant thereof); and a LCVR that comprises: an LCDR1 comprising the amino acid sequence set forth in SEQ ID NO: 533 (or a variant thereof), an LCDR2 comprising the amino acid sequence set forth in SEQ ID NO: 534 (or a variant thereof), and an LCDR3 comprising the amino acid sequence set forth in SEQ ID NO: 535 (or a variant thereof). In some such methods, the anti-TfR antigen binding protein comprises: (a) a HCVR that comprises: an HCDR1 comprising the amino acid sequence set forth in SEQ ID NO: 438 (or a variant thereof), an HCDR2 comprising the amino acid sequence set forth in SEQ ID NO: 439 (or a variant thereof), and an HCDR3 comprising the amino acid sequence set forth in SEQ ID NO: 440 (or a variant thereof); and a LCVR that comprises: an LCDR1 comprising the amino acid sequence set forth in SEQ ID NO: 443 (or a variant thereof), an LCDR2 comprising the amino acid sequence set forth in SEQ ID NO: 444 (or a variant thereof), and an LCDR3 comprising the amino acid sequence set forth in SEQ ID NO: 445 (or a variant thereof); or (b) a HCVR that comprises: an HCDR1 comprising the amino acid sequence set forth in SEQ ID NO: 458 (or a variant thereof), an HCDR2 comprising the amino acid sequence set forth in SEQ ID NO: 459 (or a variant thereof), and an HCDR3 comprising the amino acid sequence set forth in SEQ ID NO: 460 (or a variant thereof); and a LCVR that comprises: an LCDR1 comprising the amino acid sequence set forth in SEQ ID NO: 463 (or a variant thereof), an LCDR2 comprising the amino acid sequence set forth in SEQ ID NO: 464 (or a variant thereof), and an LCDR3 comprising the amino acid sequence set forth in SEQ ID NO: 465 (or a variant thereof). In some such methods, the anti-TfR antigen binding protein comprises: (i) a HCVR that comprises the amino acid sequence set forth in SEQ ID NO: 217 (or a variant thereof); and a LCVR that comprises the amino acid sequence set forth in SEQ ID NO: 222 (or a variant thereof); (ii) a HCVR that comprises the amino acid sequence set forth in SEQ ID NO: 227 (or a variant thereof); and a LCVR that comprises the amino acid sequence set forth in SEQ ID NO: 232 (or a variant thereof); (iii) a HCVR that comprises the amino acid sequence set forth in SEQ ID NO: 237 (or a variant thereof); and a LCVR that comprises the amino acid sequence set forth in SEQ ID NO: 242 (or a variant thereof); (iv) a HCVR that comprises the amino acid sequence set forth in SEQ ID NO: 247 (or a variant thereof); and a LCVR that comprises the amino acid sequence set forth in SEQ ID NO: 252 (or a variant thereof); (v) a HCVR that comprises the amino acid sequence set forth in SEQ ID NO: 257 (or a variant thereof); and a LCVR that comprises the amino acid sequence set forth in SEQ ID NO: 262 (or a variant thereof); (vi) a HCVR that comprises the amino acid sequence set forth in SEQ ID NO: 267 (or a variant thereof); and a LCVR that comprises the amino acid sequence set forth in SEQ ID NO: 272 (or a variant thereof); (vii) a HCVR that comprises the amino acid sequence set forth in SEQ ID NO: 277 (or a variant thereof); and a LCVR that comprises the amino acid sequence set forth in SEQ ID NO: 282 (or a variant thereof); (viii) a HCVR that comprises the amino acid sequence set forth in SEQ ID NO: 287 (or a variant thereof); and a LCVR that comprises the amino acid sequence set forth in SEQ ID NO: 292 (or a variant thereof); (ix) a HCVR that comprises the amino acid sequence set forth in SEQ ID NO: 297 (or a variant thereof); and a LCVR that comprises the amino acid sequence set forth in SEQ ID NO: 302 (or a variant thereof); (x) a HCVR that comprises the amino acid sequence set forth in SEQ ID NO: 307 (or a variant thereof); and a LCVR that comprises the amino acid sequence set forth in SEQ ID NO: 312 (or a variant thereof); (xi) a HCVR that comprises the amino acid sequence set forth in SEQ ID NO: 317 (or a variant thereof); and a LCVR that comprises the amino acid sequence set forth in SEQ ID NO: 322 (or a variant thereof); (xii) a HCVR that comprises the amino acid sequence set forth in SEQ ID NO: 327 (or a variant thereof); and a LCVR that comprises the amino acid sequence set forth in SEQ ID NO: 332 (or a variant thereof); (xiii) a HCVR that comprises the amino acid sequence set forth in SEQ ID NO: 337 (or a variant thereof); and a LCVR that comprises the amino acid sequence set forth in SEQ ID NO: 342 (or a variant thereof); (xiv) a HCVR that comprises the amino acid sequence set forth in SEQ ID NO: 347 (or a variant thereof); and a LCVR that comprises the amino acid sequence set forth in SEQ ID NO: 352 (or a variant thereof); (xv) a HCVR that comprises the amino acid sequence set forth in SEQ ID NO: 357 (or a variant thereof); and a LCVR that comprises the amino acid sequence set forth in SEQ ID NO: 362 (or a variant thereof); (xvi) a HCVR that comprises the amino acid sequence set forth in SEQ ID NO: 367 (or a variant thereof); and a LCVR that comprises the amino acid sequence set forth in SEQ ID NO: 372 (or a variant thereof); (xvii) a HCVR that comprises the amino acid sequence set forth in SEQ ID NO: 377 (or a variant thereof); and a LCVR that comprises the amino acid sequence set forth in SEQ ID NO: 382 (or a variant thereof); (xviii) a HCVR that comprises the amino acid sequence set forth in SEQ ID NO: 387 (or a variant thereof); and a LCVR that comprises the amino acid sequence set forth in SEQ ID NO: 392 (or a variant thereof); (xix) a HCVR that comprises the amino acid sequence set forth in SEQ ID NO: 397 (or a variant thereof); and a LCVR that comprises the amino acid sequence set forth in SEQ ID NO: 402 (or a variant thereof); (xx) a HCVR that comprises the amino acid sequence set forth in SEQ ID NO: 407 (or a variant thereof); and a LCVR that comprises the amino acid sequence set forth in SEQ ID NO: 412 (or a variant thereof); (xxi) a HCVR that comprises the amino acid sequence set forth in SEQ ID NO: 417 (or a variant thereof); and a LCVR that comprises the amino acid sequence set forth in SEQ ID NO: 422 (or a variant thereof); (xxii) a HCVR that comprises the amino acid sequence set forth in SEQ ID NO: 427 (or a variant thereof); and a LCVR that comprises the amino acid sequence set forth in SEQ ID NO: 432 (or a variant thereof); (xxiii) a HCVR that comprises the amino acid sequence set forth in SEQ ID NO: 437 (or a variant thereof); and a LCVR that comprises the amino acid sequence set forth in SEQ ID NO: 442 (or a variant thereof); (xxiv) a HCVR that comprises the amino acid sequence set forth in SEQ ID NO: 447 (or a variant thereof); and a LCVR that comprises the amino acid sequence set forth in SEQ ID NO: 452 (or a variant thereof); (xxv) a HCVR that comprises the amino acid sequence set forth in SEQ ID NO: 457 (or a variant thereof); and a LCVR that comprises the amino acid sequence set forth in SEQ ID NO: 462 (or a variant thereof); (xxvi) a HCVR that comprises the amino acid sequence set forth in SEQ ID NO: 467 (or a variant thereof); and a LCVR that comprises the amino acid sequence set forth in SEQ ID NO: 472 (or a variant thereof); (xxvii) a HCVR that comprises the amino acid sequence set forth in SEQ ID NO: 477 (or a variant thereof); and a LCVR that comprises the amino acid sequence set forth in SEQ ID NO: 482 (or a variant thereof); (xxviii) a HCVR that comprises the amino acid sequence set forth in SEQ ID NO: 487 (or a variant thereof); and a LCVR that comprises the amino acid sequence set forth in SEQ ID NO: 492 (or a variant thereof); (xxix) a HCVR that comprises the amino acid sequence set forth in SEQ ID NO: 497 (or a variant thereof); and a LCVR that comprises the amino acid sequence set forth in SEQ ID NO: 502 (or a variant thereof); (xxx) a HCVR that comprises the amino acid sequence set forth in SEQ ID NO: 507 (or a variant thereof); and a LCVR that comprises the amino acid sequence set forth in SEQ ID NO: 512 (or a variant thereof); (xxxi) a HCVR that comprises the amino acid sequence set forth in SEQ ID NO: 517 (or a variant thereof); and a LCVR that comprises the amino acid sequence set forth in SEQ ID NO: 522 (or a variant thereof); and/or (xxxii) a HCVR that comprises the amino acid sequence set forth in SEQ ID NO: 527 (or a variant thereof); and a LCVR that comprises the amino acid sequence set forth in SEQ ID NO: 532 (or a variant thereof). In some such methods, the anti-TfR antigen binding protein comprises: (i) a HCVR that comprises the amino acid sequence set forth in SEQ ID NO: 437 (or a variant thereof); and a LCVR that comprises the amino acid sequence set forth in SEQ ID NO: 442 (or a variant thereof); or (ii) a HCVR that comprises the amino acid sequence set forth in SEQ ID NO: 457 (or a variant thereof); and a LCVR that comprises the amino acid sequence set forth in SEQ ID NO: 462 (or a variant thereof). In some such methods, the TfR-binding delivery domain comprises an anti-TfR antibody, antibody fragment, or single-chain variable fragment (scFv). In some such methods, the TfR-binding delivery domain is the single-chain variable fragment (scFv), optionally wherein the multidomain therapeutic protein comprises domains arranged in the following orientation: N′-Heavy chain variable region-Light chain variable region-lysosomal alpha-glucosidase-C′ or N′-Light chain variable region-Heavy chain variable region-lysosomal alpha-glucosidase-C′, optionally wherein the scFv and lysosomal alpha-glucosidase are connected by a peptide linker, and optionally wherein the peptide linker which is -(GGGGS)ₘ- (SEQ ID NO: 600); wherein m is 1, 2, 3, 4, 5, 6, 7, 8, 9, or 10, optionally wherein the scFv variable regions are connected by a peptide linker, and optionally wherein the peptide linker which is -(GGGGS)ₘ- (SEQ ID NO: 600); wherein m is 1, 2, 3, 4, 5, 6, 7, 8, 9, or 10. In some such methods, the multidomain therapeutic protein comprises a heavy chain variable region (V_{H}) and a light chain variable region (V_{L}), and a lysosomal alpha-glucosidase, wherein the V_{H}, V_{L} and lysosomal alpha-glucosidase are arranged as follows: (i) V_{L}-V_{H}-lysosomal alpha-glucosidase; (ii) V_{H}-V_{L}-lysosomal alpha-glucosidase; (iii) V_{L}-[(GGGGS)₃]-V_{H}-[(GGGGS)₂]-lysosomal alpha-glucosidase; or V_{H}-[(GGGGS)₃]-V_{L}-[(GGGGS)₂]-lysosomal alpha-glucosidase. In some such methods, the scFv comprises the sequence set forth in any one of SEQ ID NOS: 540, 549, 551, and 554, optionally wherein the scFv comprises the sequence set forth in SEQ ID NO: 554. In some such methods, the scFv consists of the sequence set forth in any one of SEQ ID NOS: 540, 549, 551, and 554, optionally wherein the scFv consists of the sequence set forth in SEQ ID NOS: 554. In some such methods, the scFv coding sequence is at least 90%, at least 91%, at least 92%, at least 93%, at least 94%, at least 95%, at least 96%, at least 97%, at least 98%, or at least 99% identical to any one of SEQ ID NOS: 587-599. Optionally, the scFv coding sequence is at least 90%, at least 91%, at least 92%, at least 93%, at least 94%, at least 95%, at least 96%, at least 97%, at least 98%, or at least 99% identical to any one of SEQ ID NOS: 593-595, optionally wherein the scFv coding sequence is at least 90%, at least 91%, at least 92%, at least 93%, at least 94%, at least 95%, at least 96%, at least 97%, at least 98%, or at least 99% identical to SEQ ID NO: 593. Optionally the scFv coding sequence is at least 90%, at least 91%, at least 92%, at least 93%, at least 94%, at least 95%, at least 96%, at least 97%, at least 98%, or at least 99% identical to any one of SEQ ID NOS: 590-592, optionally wherein the scFv coding sequence is at least 90%, at least 91%, at least 92%, at least 93%, at least 94%, at least 95%, at least 96%, at least 97%, at least 98%, or at least 99% identical to SEQ ID NO: 590. In some such methods, the scFv coding sequence is at least 90%, at least 91%, at least 92%, at least 93%, at least 94%, at least 95%, at least 96%, at least 97%, at least 98%, or at least 99% identical to any one of SEQ ID NOS: 587-599 and encodes an scFv comprising any one of SEQ ID NOS: 540, 549, 551, and 554. Optionally, the scFv coding sequence is at least 90%, at least 91%, at least 92%, at least 93%, at least 94%, at least 95%, at least 96%, at least 97%, at least 98%, or at least 99% identical to any one of SEQ ID NOS: 593-595 and encodes an scFv comprising SEQ ID NO: 554, optionally wherein the scFv coding sequence is at least 90%, at least 91%, at least 92%, at least 93%, at least 94%, at least 95%, at least 96%, at least 97%, at least 98%, or at least 99% identical to SEQ ID NO: 593 and encodes an scFv comprising SEQ ID NO: 554. Optionally, the scFv coding sequence is at least 90%, at least 91%, at least 92%, at least 93%, at least 94%, at least 95%, at least 96%, at least 97%, at least 98%, or at least 99% identical to any one of SEQ ID NOS: 590-592 and encodes an scFv comprising SEQ ID NO: 551, optionally wherein the scFv coding sequence is at least 90%, at least 91%, at least 92%, at least 93%, at least 94%, at least 95%, at least 96%, at least 97%, at least 98%, or at least 99% identical to SEQ ID NO: 590 and encodes an scFv comprising SEQ ID NO: 551. In some such methods, the scFv coding sequence is at least 90%, at least 91%, at least 92%, at least 93%, at least 94%, at least 95%, at least 96%, at least 97%, at least 98%, or at least 99% identical to any one of SEQ ID NOS: 587-599, is codon-optimized and CpG-depleted, and encodes an scFv comprising any one of SEQ ID NOS: 540, 549, 551, and 554. Optionally, the scFv coding sequence is at least 90%, at least 91%, at least 92%, at least 93%, at least 94%, at least 95%, at least 96%, at least 97%, at least 98%, or at least 99% identical to any one of SEQ ID NOS: 593-595, is codon-optimized and CpG-depleted, and encodes an scFv comprising SEQ ID NO: 554, optionally wherein the scFv coding sequence is at least 90%, at least 91%, at least 92%, at least 93%, at least 94%, at least 95%, at least 96%, at least 97%, at least 98%, or at least 99% identical to SEQ ID NO: 593, the scFv coding sequence is codon-optimized and CpG-depleted, and encodes an scFv comprising SEQ ID NO: 554. Optionally, the scFv coding sequence is at least 90%, at least 91%, at least 92%, at least 93%, at least 94%, at least 95%, at least 96%, at least 97%, at least 98%, or at least 99% identical to any one of SEQ ID NOS: 590-592, is codon-optimized and CpG-depleted, and encodes an scFv comprising SEQ ID NO: 551, optionally wherein the scFv coding sequence is at least 90%, at least 91%, at least 92%, at least 93%, at least 94%, at least 95%, at least 96%, at least 97%, at least 98%, or at least 99% identical to SEQ ID NO: 590, the scFv coding sequence is codon-optimized and CpG-depleted, and encodes an scFv comprising SEQ ID NO: 551. In some such methods, the scFv coding sequence comprises the sequence set forth in any one of SEQ ID NOS: 587-599. Optionally, the scFv coding sequence comprises the sequence set forth in any one of SEQ ID NOS: 593-595, optionally wherein the scFv coding sequence comprises the sequence set forth in SEQ ID NO: 593. Optionally, the scFv coding sequence comprises the sequence set forth in any one of SEQ ID NOS: 590-592, optionally wherein the scFv coding sequence comprises the sequence set forth in SEQ ID NO: 590. In some such methods, the scFv coding sequence consists of the sequence set forth in any one of SEQ ID NOS: 587-599. Optionally, the scFv coding sequence consists of the sequence set forth in any one of SEQ ID NOS: 593-595, optionally wherein the scFv coding sequence consists of the sequence set forth in SEQ ID NO: 593. Optionally, the scFv coding sequence consists of the sequence set forth in any one of SEQ ID NOS: 590-592, optionally wherein the scFv coding sequence consists of the sequence set forth in SEQ ID NO: 590.

In some such methods, the lysosomal alpha-glucosidase lacks the lysosomal alpha-glucosidase signal peptide and propeptide. In some such methods, the lysosomal alpha-glucosidase comprises the sequence set forth in SEQ ID NO: 173. In some such methods, the lysosomal alpha-glucosidase consists of the sequence set forth in SEQ ID NO: 173. In some such methods, the lysosomal alpha-glucosidase coding sequence is codon-optimized or CpG-depleted. In some such methods, the lysosomal alpha-glucosidase coding sequence is codon-optimized and CpG-depleted. In some such methods, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and 205-212, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176. In some such methods, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and 205-212 and encodes a lysosomal alpha-glucosidase protein comprising SEQ ID NO: 173, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176 and encodes a lysosomal alpha-glucosidase protein comprising SEQ ID NO: 173. In some such methods, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and 205-212, is codon-optimized and CpG-depleted, and encodes a lysosomal alpha-glucosidase protein comprising SEQ ID NO: 173, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176, is codon-optimized and CpG-depleted, and encodes a lysosomal alpha-glucosidase protein comprising SEQ ID NO: 173. In some such methods, the lysosomal alpha-glucosidase coding sequence comprises the sequence set forth in any one of SEQ ID NOS: 174-182 and 205-212, optionally wherein the lysosomal alpha-glucosidase coding sequence comprises the sequence set forth in SEQ ID NO: 176. In some such methods, the lysosomal alpha-glucosidase coding sequence consists of the sequence set forth in any one of SEQ ID NOS: 174-182 and 205-212, optionally wherein the lysosomal alpha-glucosidase coding sequence consists of the sequence set forth in SEQ ID NO: 176.

In some such methods, the lysosomal alpha-glucosidase lacks the lysosomal alpha-glucosidase signal peptide and propeptide. In some such methods, the lysosomal alpha-glucosidase comprises the sequence set forth in SEQ ID NO: 173. In some such methods, the lysosomal alpha-glucosidase consists of the sequence set forth in SEQ ID NO: 173. In some such methods, the lysosomal alpha-glucosidase coding sequence is codon-optimized or CpG-depleted. In some such methods, the lysosomal alpha-glucosidase coding sequence is codon-optimized and CpG-depleted. In some such methods, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176. In some such methods, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and encodes a lysosomal alpha-glucosidase protein comprising SEQ ID NO: 173, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176 and encodes a lysosomal alpha-glucosidase protein comprising SEQ ID NO: 173. In some such methods, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182, is codon-optimized and CpG-depleted, and encodes a lysosomal alpha-glucosidase protein comprising SEQ ID NO: 173, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176, is codon-optimized and CpG-depleted, and encodes a lysosomal alpha-glucosidase protein comprising SEQ ID NO: 173. In some such methods, the lysosomal alpha-glucosidase coding sequence comprises the sequence set forth in any one of SEQ ID NOS: 174-182, optionally wherein the lysosomal alpha-glucosidase coding sequence comprises the sequence set forth in SEQ ID NO: 176. In some such methods, the lysosomal alpha-glucosidase coding sequence consists of the sequence set forth in any one of SEQ ID NOS: 174-182, optionally wherein the lysosomal alpha-glucosidase coding sequence consists of the sequence set forth in SEQ ID NO: 176.

In some such methods, the coding sequence for the multidomain therapeutic protein is codon-optimized or CpG-depleted. In some such methods, the coding sequence for the multidomain therapeutic protein is codon-optimized and CpG-depleted. In some such methods, the multidomain therapeutic protein comprises the sequence set forth in any one of SEQ ID NOS: 570-573, optionally wherein the multidomain therapeutic protein comprises the sequence set forth in SEQ ID NO: 573. In some such methods, the multidomain therapeutic protein consists of the sequence set forth in any one of SEQ ID NOS: 570-573, optionally wherein the multidomain therapeutic protein consists of the sequence set forth in SEQ ID NO: 573. In some such methods, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 574-586. Optionally, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 584-586, optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 584, optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 733. Optionally, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 581-583, optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 581, optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 729.In some such methods, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 574-586, and the multidomain therapeutic protein comprises the sequence set forth in any one of SEQ ID NOS: 570-573. Optionally, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 584-586, and the multidomain therapeutic protein comprises the sequence set forth in SEQ ID NO: 573, optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 584, and the multidomain therapeutic protein comprises the sequence set forth in SEQ ID NO: 573, optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 733, and the multidomain therapeutic protein comprises the sequence set forth in SEQ ID NO: 573. Optionally, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 581-583, and the multidomain therapeutic protein comprises the sequence set forth in SEQ ID NO: 572, optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 581, and the multidomain therapeutic protein comprises the sequence set forth in SEQ ID NO: 572, optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 729, and the multidomain therapeutic protein comprises the sequence set forth in SEQ ID NO: 572. In some such methods, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 574-586 and is codon-optimized and CpG-depleted, and the multidomain therapeutic protein comprises the sequence set forth in any one of SEQ ID NOS: 570-573. Optionally, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 584-586 and is codon-optimized and CpG-depleted, and the multidomain therapeutic protein comprises the sequence set forth in SEQ ID NO: 573, optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 584, the coding sequence for the multidomain therapeutic protein is codon-optimized and CpG-depleted, and the multidomain therapeutic protein comprises the sequence set forth in SEQ ID NO: 573, optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 733, the coding sequence for the multidomain therapeutic protein is codon-optimized and CpG-depleted, and the multidomain therapeutic protein comprises the sequence set forth in SEQ ID NO: 573. Optionally, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 581-583 and is codon-optimized and CpG-depleted, and the multidomain therapeutic protein comprises the sequence set forth in SEQ ID NO: 572, optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 581, the coding sequence for the multidomain therapeutic protein is codon-optimized and CpG-depleted, and the multidomain therapeutic protein comprises the sequence set forth in SEQ ID NO: 572, optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 729, the coding sequence for the multidomain therapeutic protein is codon-optimized and CpG-depleted, and the multidomain therapeutic protein comprises the sequence set forth in SEQ ID NO: 572. In some such methods, the coding sequence for the multidomain therapeutic protein comprises the sequence set forth in any one of SEQ ID NOS: 574-586. Optionally, the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733. Optionally, the coding sequence for the multidomain therapeutic protein comprises the sequence set forth in any one of SEQ ID NOS: 581-583, optionally wherein the coding sequence for the multidomain therapeutic protein comprises the sequence set forth in SEQ ID NO: 581, optionally wherein the nucleic acid construct comprises the sequence set forth in SEQ ID NO: 729. In some such methods, the coding sequence for the multidomain therapeutic protein consists of the sequence set forth in any one of SEQ ID NOS: 574-586. Optionally, the coding sequence for the multidomain therapeutic protein consists of the sequence set forth in any one of SEQ ID NOS: 584-586, optionally wherein the coding sequence for the multidomain therapeutic protein consists of the sequence set forth in SEQ ID NO: 584, optionally wherein the nucleic acid construct comprises the sequence set forth in SEQ ID NO: 733. Optionally, the coding sequence for the multidomain therapeutic protein consists of the sequence set forth in any one of SEQ ID NOS: 581-583, optionally wherein the coding sequence for the multidomain therapeutic protein consists of the sequence set forth in SEQ ID NO: 581, optionally wherein the nucleic acid construct comprises the sequence set forth in SEQ ID NO: 729. In some such methods, the nucleic acid construct comprises a splice acceptor upstream of the coding sequence for the multidomain therapeutic protein. In some such methods, the nucleic acid construct comprises a polyadenylation signal or sequence downstream of the coding sequence for the multidomain therapeutic protein. In some such methods, the nucleic acid construct comprises a splice acceptor upstream of the coding sequence for the multidomain therapeutic protein, and the nucleic acid construct comprises a polyadenylation signal or sequence downstream of the coding sequence for the multidomain therapeutic protein. In some such methods, the nucleic acid construct does not comprise a homology arm. In some such methods, the nucleic acid construct is inserted into the target genomic locus via non-homologous end joining. In some such methods, the nucleic acid construct comprises homology arms. In some such methods, the nucleic acid construct is inserted into the target genomic locus via homology-directed repair. In some such methods, the nucleic acid construct does not comprise a promoter that drives the expression of the multidomain therapeutic protein. In some such methods, the coding sequence for the multidomain therapeutic protein is operably linked to a promoter, optionally wherein the promoter is a liver-specific promoter. In some such methods, the nucleic acid construct is single-stranded DNA or doublestranded DNA. In some such methods, the nucleic acid construct is single-stranded DNA. In some such methods, the nucleic acid construct comprises from 5′ to 3′: a splice acceptor, the coding sequence for the multidomain therapeutic protein, and a polyadenylation signal or sequence, wherein the lysosomal alpha-glucosidase coding sequence comprises the sequence set forth in any one of SEQ ID NOS: 174-182 and 205-212, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733, or optionally wherein the coding sequence for the multidomain therapeutic protein comprises the sequence set forth in any one of SEQ ID NOS: 581-583, optionally wherein the coding sequence for the multidomain therapeutic protein comprises the sequence set forth in SEQ ID NO: 581, optionally wherein the nucleic acid construct comprises the sequence set forth in SEQ ID NO: 729, wherein the nucleic acid construct does not comprise a promoter that drives the expression of the multidomain therapeutic protein, and wherein the nucleic acid construct does not comprise a homology arm. In some such methods, the nucleic acid construct comprises from 5′ to 3′: a splice acceptor, the coding sequence for the multidomain therapeutic protein, and a polyadenylation signal or sequence, wherein the lysosomal alpha-glucosidase coding sequence comprises the sequence set forth in any one of SEQ ID NOS: 174-182, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733, or optionally wherein the coding sequence for the multidomain therapeutic protein comprises the sequence set forth in any one of SEQ ID NOS: 581-583, optionally wherein the coding sequence for the multidomain therapeutic protein comprises the sequence set forth in SEQ ID NO: 581, optionally wherein the nucleic acid construct comprises the sequence set forth in SEQ ID NO: 729, wherein the nucleic acid construct does not comprise a promoter that drives the expression of the multidomain therapeutic protein, and wherein the nucleic acid construct does not comprise a homology arm.

In some such methods, the nucleic acid construct is in a nucleic acid vector or a lipid nanoparticle. In some such methods, the nucleic acid construct is in the nucleic acid vector. In some such methods, the nucleic acid vector is a viral vector. In some such methods, the nucleic acid vector is an adeno-associated viral (AAV) vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160. In some such methods, the AAV vector is a single-stranded AAV (ssAAV) vector. In some such methods, the AAV vector is derived from an AAV8 vector, an AAV3B vector, an AAV5 vector, an AAV6 vector, an AAV7 vector, an AAV9 vector, an AAVrh.74 vector, or an AAVhu.37 vector. In some such methods, the AAV vector is a recombinant AAV8 (rAAV8) vector. In some such methods, the AAV vector is a single-stranded rAAV8 vector. In some such methods, the nucleic acid construct comprises from 5′ to 3′: a splice acceptor, the coding sequence for the multidomain therapeutic protein, and a polyadenylation signal or sequence, wherein the lysosomal alpha-glucosidase coding sequence comprises the sequence set forth in any one of SEQ ID NOS: 174-182 and 205-212, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733, or optionally wherein the coding sequence for the multidomain therapeutic protein comprises the sequence set forth in any one of SEQ ID NOS: 581-583, optionally wherein the coding sequence for the multidomain therapeutic protein comprises the sequence set forth in SEQ ID NO: 581, optionally wherein the nucleic acid construct comprises the sequence set forth in SEQ ID NO: 729, wherein the nucleic acid construct does not comprise a promoter that drives the expression of the multidomain therapeutic protein, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160. In some such methods, the nucleic acid construct comprises from 5′ to 3′: a splice acceptor, the coding sequence for the multidomain therapeutic protein, and a polyadenylation signal or sequence, wherein the lysosomal alpha-glucosidase coding sequence comprises the sequence set forth in any one of SEQ ID NOS: 174-182, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733, or optionally wherein the coding sequence for the multidomain therapeutic protein comprises the sequence set forth in any one of SEQ ID NOS: 581-583, optionally wherein the coding sequence for the multidomain therapeutic protein comprises the sequence set forth in SEQ ID NO: 581, optionally wherein the nucleic acid construct comprises the sequence set forth in SEQ ID NO: 729, wherein the nucleic acid construct does not comprise a promoter that drives the expression of the multidomain therapeutic protein, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160. In some such methods, the nucleic acid construct is CpG-depleted.

In some such methods, the target genomic locus is an albumin gene, optionally wherein the albumin gene is a human albumin gene. In some such methods, the nuclease target site is in intron 1 of the albumin gene. In some such methods, the nuclease agent comprises: (a) a zinc finger nuclease (ZFN); (b) a transcription activator-like effector nuclease (TALEN); or (c) (i) a Cas protein or a nucleic acid encoding the Cas protein; and (ii) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

In some such methods, the nuclease agent comprises: (a) a Cas protein or a nucleic acid encoding the Cas protein; and (b) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence. In some such methods, the guide RNA target sequence is in intron 1 of an albumin gene. In some such methods, the albumin gene is a human albumin gene. In some such methods, the DNA-targeting segment comprises at least 17, at least 18, at least 19, or at least 20 contiguous nucleotides of the sequence set forth in any one of SEQ ID NOS: 30-61, optionally wherein the DNA-targeting segment comprises at least 17, at least 18, at least 19, or at least 20 contiguous nucleotides of the sequence set forth in any one of SEQ ID NOS: 36, 30, 33, and 41. In some such methods, the DNA-targeting segment is at least 90% or at least 95% identical to the sequence set forth in any one of SEQ ID NOS: 30-61, optionally wherein the DNA-targeting segment is at least 90% or at least 95% identical to the sequence set forth in any one of SEQ ID NOS: 36, 30, 33, and 41. In some such methods, the DNA-targeting segment comprises any one of SEQ ID NOS: 30-61, optionally wherein the DNA-targeting segment comprises any one of SEQ ID NOS: 36, 30, 33, and 41. In some such methods, the DNA-targeting segment consists of any one of SEQ ID NOS: 30-61, optionally wherein the DNA-targeting segment consists of any one of SEQ ID NOS: 36, 30, 33, and 41. In some such methods, the guide RNA comprises any one of SEQ ID NOS: 62-125, optionally wherein the guide RNA comprises any one of SEQ ID NOS: 68, 100, 62, 94, 65, 97, 73, and 105. In some such methods, the DNA-targeting segment comprises at least 17, at least 18, at least 19, or at least 20 contiguous nucleotides of SEQ ID NO: 36. In some such methods, the DNA-targeting segment is at least 90% or at least 95% identical to SEQ ID NO: 36. In some such methods, the DNA-targeting segment comprises SEQ ID NO: 36. In some such methods, the DNA-targeting segment consists of SEQ ID NO: 36. In some such methods, the guide RNA comprises SEQ ID NO: 68 or 100.

In some such methods, the method comprises administering the guide RNA in the form of RNA. In some such methods, the guide RNA comprises at least one modification. In some such methods, the at least one modification comprises a 2′-O-methyl-modified nucleotide. In some such methods, the at least one modification comprises a phosphorothioate bond between nucleotides. In some such methods, the at least one modification comprises a modification at one or more of the first five nucleotides at the 5′ end of the guide RNA. In some such methods, the at least one modification comprises a modification at one or more of the last five nucleotides at the 3′ end of the guide RNA. In some such methods, the at least one modification comprises phosphorothioate bonds between the first four nucleotides at the 5′ end of the guide RNA. In some such methods, the at least one modification comprises phosphorothioate bonds between the last four nucleotides at the 3′ end of the guide RNA. In some such methods, the at least one modification comprises 2′-O-methyl-modified nucleotides at the first three nucleotides at the 5′ end of the guide RNA. In some such methods, the at least one modification comprises 2′-O-methyl-modified nucleotides at the last three nucleotides at the 3′ end of the guide RNA. In some such methods, the at least one modification comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA. In some such methods, the guide RNA is a single guide RNA (sgRNA). In some such methods, the method comprises administering the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

In some such methods, the Cas protein is a Cas9 protein. In some such methods, the Cas9 protein is derived from a Streptococcus pyogenes Cas9 protein, a Staphylococcus aureus Cas9 protein, a Campylobacter jejuni Cas9 protein, a Streptococcus thermophilus Cas9 protein, or a Neisseria meningitidis Cas9 protein. In some such methods, the Cas protein is derived from a Streptococcus pyogenes Cas9 protein. In some such methods, the Cas protein comprises the sequence set forth in SEQ ID NO: 11. In some such methods, the nucleic acid encoding the Cas protein is codon-optimized for expression in a mammalian cell or a human cell. In some such methods, the method comprises administering the nucleic acid encoding the Cas protein, wherein the nucleic acid comprises an mRNA encoding the Cas protein. In some such methods, the mRNA encoding the Cas protein comprises at least one modification. In some such methods, the mRNA encoding the Cas protein is modified to comprise a modified uridine at one or more or all uridine positions. In some such methods, the modified uridine is pseudouridine or N1-methyl-pseudouridine, optionally N1-methyl-pseudouridine. In some such methods, the mRNA encoding the Cas protein is fully substituted with pseudouridine or N1-methyl-pseudouridine, optionally N1-methyl-pseudouridine. In some such methods, the modified uridine is pseudouridine. In some such methods, the mRNA encoding the Cas protein is fully substituted with pseudouridine. In some such methods, the mRNA encoding the Cas protein comprises a 5′ cap. In some such methods, the mRNA encoding the Cas protein comprises a polyadenylation sequence. In some such methods, the mRNA encoding the Cas protein comprises the sequence set forth in SEQ ID NO: 226, 225, or 12. In some such methods, the method comprises administering the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine or N1-methyl-pseudouridine, optionally N1-methyl-pseudouridine, comprises a 5′ cap, and comprises a polyadenylation sequence. In some such methods, the method comprises administering the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine, comprises a 5′ cap, and comprises a polyadenylation sequence.

In some such methods, the method comprises administering the guide RNA in the form of RNA, and the guide RNA comprises SEQ ID NO: 68 or 100, and wherein the method comprises administering the nucleic acid encoding the Cas protein, wherein the nucleic acid comprises an mRNA encoding the Cas protein, and the mRNA encoding the Cas protein comprises the sequence set forth in SEQ ID NO: 226, 225, or 12. In some such methods, the nucleic acid construct comprises from 5′ to 3′: a splice acceptor, the coding sequence for the multidomain therapeutic protein, and a polyadenylation signal or sequence, wherein the lysosomal alpha-glucosidase coding sequence comprises the sequence set forth in any one of SEQ ID NOS: 174-182 and 205-212, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733, or optionally wherein the coding sequence for the multidomain therapeutic protein comprises the sequence set forth in any one of SEQ ID NOS: 581-583, optionally wherein the coding sequence for the multidomain therapeutic protein comprises the sequence set forth in SEQ ID NO: 581, optionally wherein the nucleic acid construct comprises the sequence set forth in SEQ ID NO: 729, wherein the nucleic acid construct does not comprise a promoter that drives the expression of the multidomain therapeutic protein, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160. In some such methods, the nucleic acid construct comprises from 5′ to 3′: a splice acceptor, the coding sequence for the multidomain therapeutic protein, and a polyadenylation signal or sequence, wherein the lysosomal alpha-glucosidase coding sequence comprises the sequence set forth in any one of SEQ ID NOS: 174-182, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733, or optionally wherein the coding sequence for the multidomain therapeutic protein comprises the sequence set forth in any one of SEQ ID NOS: 581-583, optionally wherein the coding sequence for the multidomain therapeutic protein comprises the sequence set forth in SEQ ID NO: 581, optionally wherein the nucleic acid construct comprises the sequence set forth in SEQ ID NO: 729, wherein the nucleic acid construct does not comprise a promoter that drives the expression of the multidomain therapeutic protein, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160. In some such methods, the method comprises administering the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the method comprises administering the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine or N1-methyl-pseudouridine, optionally N1-methyl-pseudouridine, comprises a 5′ cap, and comprises a polyadenylation sequence. In some such methods, the method comprises administering the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the method comprises administering the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine, comprises a 5′ cap, and comprises a polyadenylation sequence. In some such methods, the nucleic acid construct comprises from 5′ to 3′: a splice acceptor, the coding sequence for the multidomain therapeutic protein, and a polyadenylation signal or sequence, wherein the lysosomal alpha-glucosidase coding sequence comprises the sequence set forth in any one of SEQ ID NOS: 174-182 and 205-212, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733, or optionally wherein the coding sequence for the multidomain therapeutic protein comprises the sequence set forth in any one of SEQ ID NOS: 581-583, optionally wherein the coding sequence for the multidomain therapeutic protein comprises the sequence set forth in SEQ ID NO: 581, optionally wherein the nucleic acid construct comprises the sequence set forth in SEQ ID NO: 729, wherein the nucleic acid construct does not comprise a promoter that drives the expression of the multidomain therapeutic protein, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160. In some such methods, the nucleic acid construct comprises from 5′ to 3′: a splice acceptor, the coding sequence for the multidomain therapeutic protein, and a polyadenylation signal or sequence, wherein the lysosomal alpha-glucosidase coding sequence comprises the sequence set forth in any one of SEQ ID NOS: 174-182, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733, or optionally wherein the coding sequence for the multidomain therapeutic protein comprises the sequence set forth in any one of SEQ ID NOS: 581-583, optionally wherein the coding sequence for the multidomain therapeutic protein comprises the sequence set forth in SEQ ID NO: 581, optionally wherein the nucleic acid construct comprises the sequence set forth in SEQ ID NO: 729, wherein the nucleic acid construct does not comprise a promoter that drives the expression of the multidomain therapeutic protein, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

In some such methods, the Cas protein or the nucleic acid encoding the Cas protein and the guide RNA or the one or more DNAs encoding the guide RNA are associated with a lipid nanoparticle. In some such methods, the lipid nanoparticle comprises a cationic lipid, a neutral lipid, a helper lipid, and a stealth lipid. In some such methods, the cationic lipid is Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate). In some such methods, the neutral lipid is distearoylphosphatidylcholine or 1,2-distearoyl-sn-glycero-3-phosphocholine (DSPC). In some such methods, the helper lipid is cholesterol. In some such methods, the stealth lipid is PEG2k-DMG. In some such methods, the cationic lipid is Lipid A, the neutral lipid is DSPC, the helper lipid is cholesterol, and the stealth lipid is PEG2k-DMG. In some such methods, the lipid nanoparticle comprises four lipids at the following molar ratios: about 50 mol% Lipid A, about 9 mol% DSPC, about 38 mol% cholesterol, and about 3 mol% PEG2k-DMG.

In some such methods, the albumin gene is a human albumin gene, wherein the method comprises administering the guide RNA in the form of RNA, and the guide RNA comprises SEQ ID NO: 68 or 100, wherein the method comprises administering the nucleic acid encoding the Cas protein, wherein the nucleic acid comprises an mRNA encoding the Cas protein, and the mRNA encoding the Cas protein comprises the sequence set forth in SEQ ID NO: 226, 225, or 12, and wherein the guide RNA and the mRNA encoding the Cas protein are associated with a lipid nanoparticle comprising Lipid A, DSPC, cholesterol, and PEG2k-DMG, optionally at the following molar ratios: about 50 mol% Lipid A, about 9 mol% DSPC, about 38 mol% cholesterol, and about 3 mol% PEG2k-DMG. In some such methods, the nucleic acid construct comprises from 5′ to 3′: a splice acceptor, the coding sequence for the multidomain therapeutic protein, and a polyadenylation signal or sequence, wherein the lysosomal alpha-glucosidase coding sequence comprises the sequence set forth in any one of SEQ ID NOS: 174-182 and 205-212, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733, or optionally wherein the coding sequence for the multidomain therapeutic protein comprises the sequence set forth in any one of SEQ ID NOS: 581-583, optionally wherein the coding sequence for the multidomain therapeutic protein comprises the sequence set forth in SEQ ID NO: 581, optionally wherein the nucleic acid construct comprises the sequence set forth in SEQ ID NO: 729, wherein the nucleic acid construct does not comprise a promoter that drives the expression of the multidomain therapeutic protein, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160. In some such methods, the nucleic acid construct comprises from 5′ to 3′: a splice acceptor, the coding sequence for the multidomain therapeutic protein, and a polyadenylation signal or sequence, wherein the lysosomal alpha-glucosidase coding sequence comprises the sequence set forth in any one of SEQ ID NOS: 174-182, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733, or optionally wherein the coding sequence for the multidomain therapeutic protein comprises the sequence set forth in any one of SEQ ID NOS: 581-583, optionally wherein the coding sequence for the multidomain therapeutic protein comprises the sequence set forth in SEQ ID NO: 581, optionally wherein the nucleic acid construct comprises the sequence set forth in SEQ ID NO: 729, wherein the nucleic acid construct does not comprise a promoter that drives the expression of the multidomain therapeutic protein, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

In some such methods, the albumin gene is a human albumin gene, wherein the method comprises administering the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the method comprises administering the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine or N1-methyl-pseudouridine, optionally N1-methyl-pseudouridine, comprises a 5′ cap, and comprises a polyadenylation sequence, and wherein the guide RNA and the mRNA encoding the Cas protein are associated with a lipid nanoparticle comprising Lipid A, DSPC, cholesterol, and PEG2k-DMG, optionally at the following molar ratios: about 50 mol% Lipid A, about 9 mol% DSPC, about 38 mol% cholesterol, and about 3 mol% PEG2k-DMG. In some such methods, the albumin gene is a human albumin gene, wherein the method comprises administering the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the method comprises administering the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine, comprises a 5′ cap, and comprises a polyadenylation sequence, and wherein the guide RNA and the mRNA encoding the Cas protein are associated with a lipid nanoparticle comprising Lipid A, DSPC, cholesterol, and PEG2k-DMG, optionally at the following molar ratios: about 50 mol% Lipid A, about 9 mol% DSPC, about 38 mol% cholesterol, and about 3 mol% PEG2k-DMG. In some such methods, the nucleic acid construct comprises from 5′ to 3′: a splice acceptor, the coding sequence for the multidomain therapeutic protein, and a polyadenylation signal or sequence, wherein the lysosomal alpha-glucosidase coding sequence comprises the sequence set forth in any one of SEQ ID NOS: 174-182 and 205-212, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733, or optionally wherein the coding sequence for the multidomain therapeutic protein comprises the sequence set forth in any one of SEQ ID NOS: 581-583, optionally wherein the coding sequence for the multidomain therapeutic protein comprises the sequence set forth in SEQ ID NO: 581, optionally wherein the nucleic acid construct comprises the sequence set forth in SEQ ID NO: 729, wherein the nucleic acid construct does not comprise a promoter that drives the expression of the multidomain therapeutic protein, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160. In some such methods, the nucleic acid construct comprises from 5′ to 3′: a splice acceptor, the coding sequence for the multidomain therapeutic protein, and a polyadenylation signal or sequence, wherein the lysosomal alpha-glucosidase coding sequence comprises the sequence set forth in any one of SEQ ID NOS: 174-182, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733, optionally wherein the coding sequence for the multidomain therapeutic protein comprises the sequence set forth in any one of SEQ ID NOS: 581-583, optionally wherein the coding sequence for the multidomain therapeutic protein comprises the sequence set forth in SEQ ID NO: 581, optionally wherein the nucleic acid construct comprises the sequence set forth in SEQ ID NO: 729, wherein the nucleic acid construct does not comprise a promoter that drives the expression of the multidomain therapeutic protein, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

In another aspect, provided is a neonatal cell or a population of neonatal cells made by any of the above methods. In another aspect, provided is a neonatal cell or a population of neonatal cells comprising a nucleic acid construct inserted into a target genomic locus, wherein the nucleic acid construct comprises a coding sequence for a multidomain therapeutic protein comprising a TfR-binding delivery domain fused to a lysosomal alpha-glucosidase inserted into a target genomic locus. In some such neonatal cells or populations of neonatal cells, the neonatal cell is a liver cell or the population of neonatal cells is a population of liver cells. In some such neonatal cells or populations of neonatal cells, the neonatal cell is a hepatocyte or the population of neonatal cells is a population of hepatocytes. In some such neonatal cells or populations of neonatal cells, the neonatal cell is a human cell or the population of neonatal cells is a population of human cells. In some such neonatal cells or populations of neonatal cells, the neonatal cell or the population of neonatal cells is from a human neonatal subject within 24 weeks after birth. In some such neonatal cells or populations of neonatal cells, the neonatal cell or the population of neonatal cells is from a human neonatal subject within 12 weeks after birth. In some such neonatal cells or populations of neonatal cells, the neonatal cell or the population of neonatal cells is from a human neonatal subject within 8 weeks after birth. In some such neonatal cells or populations of neonatal cells, the neonatal cell or the population of neonatal cells is from a human neonatal subject within 4 weeks after birth. In some such neonatal cells or populations of neonatal cells, the neonatal cell is in vitro or ex vivo or the population of neonatal cells is in vitro or ex vivo. In some such neonatal cells or populations of neonatal cells, the neonatal cell is in vivo in a subject or the population of neonatal cells is in vivo.

In some such neonatal cells or populations of neonatal cells, the multidomain therapeutic protein is expressed. In some such neonatal cells or populations of neonatal cells, the TfR-binding delivery domain is fused to the lysosomal alpha-glucosidase protein via a peptide linker. In some such neonatal cells or populations of neonatal cells, the coding sequence for the TfR-binding delivery domain is codon-optimized or CpG-depleted. In some such neonatal cells or populations of neonatal cells, the coding sequence for the TfR-binding delivery domain is codon-optimized and CpG-depleted. In some such neonatal cells or populations of neonatal cells, the TfR-binding delivery domain comprises an anti-TfR antigen-binding protein. In some such neonatal cells or populations of neonatal cells, the anti-TfR antigen binding protein comprises: (i) a HCVR that comprises the HCDR1, HCDR2 and HCDR3 of a HCVR comprising the amino acid sequence set forth in SEQ ID NO: 217, 227, 237, 247, 257, 267, 277, 287, 297, 307, 317, 327, 337, 347, 357, 367, 377, 387, 397, 407, 417, 427, 437, 447, 457, 467, 477, 487, 497, 507, 517, or 527 (or a variant thereof); and/or (ii) a LCVR that comprises the LCDR1, LCDR2 and LCDR3 of a LCVR comprising the amino acid sequence set forth in SEQ ID NO: 222, 232, 242, 252, 262, 272, 282, 292, 302, 312, 322, 332, 342, 352, 362, 372, 382, 392, 402, 412, 422, 432, 442, 452, 462, 472, 482, 492, 502, 512, 522, or 532 (or a variant thereof). In some such neonatal cells or populations of neonatal cells, the anti-TfR antigen binding protein comprises: (1) a HCVR comprising the HCDR1, HCDR2 and HCDR3 of a HCVR that comprises the amino acid sequence set forth in SEQ ID NO: 217 (or a variant thereof); and a LCVR comprising the LCDR1, LCDR2 and LCDR3 of a LCVR that comprises the amino acid sequence set forth in SEQ ID NO: 222 (or a variant thereof); (2) a HCVR comprising the HCDR1, HCDR2 and HCDR3 of a HCVR that comprises the amino acid sequence set forth in SEQ ID NO: 227 (or a variant thereof); and a LCVR comprising the LCDR1, LCDR2 and LCDR3 of a LCVR that comprises the amino acid sequence set forth in SEQ ID NO: 232 (or a variant thereof); (3) a HCVR comprising the HCDR1, HCDR2 and HCDR3 of a HCVR that comprises the amino acid sequence set forth in SEQ ID NO: 237 (or a variant thereof); and a LCVR comprising the LCDR1, LCDR2 and LCDR3 of a LCVR that comprises the amino acid sequence set forth in SEQ ID NO: 242 (or a variant thereof); (4) a HCVR comprising the HCDR1, HCDR2 and HCDR3 of a HCVR that comprises the amino acid sequence set forth in SEQ ID NO: 247 (or a variant thereof); and a LCVR comprising the LCDR1, LCDR2 and LCDR3 of a LCVR that comprises the amino acid sequence set forth in SEQ ID NO: 252 (or a variant thereof); (5) a HCVR comprising the HCDR1, HCDR2 and HCDR3 of a HCVR that comprises the amino acid sequence set forth in SEQ ID NO: 257 (or a variant thereof); and a LCVR comprising the LCDR1, LCDR2 and LCDR3 of a LCVR that comprises the amino acid sequence set forth in SEQ ID NO: 262 (or a variant thereof); (6) a HCVR comprising the HCDR1, HCDR2 and HCDR3 of a HCVR that comprises the amino acid sequence set forth in SEQ ID NO: 267 (or a variant thereof); and a LCVR comprising the LCDR1, LCDR2 and LCDR3 of a LCVR that comprises the amino acid sequence set forth in SEQ ID NO: 272 (or a variant thereof); (7) a HCVR comprising the HCDR1, HCDR2 and HCDR3 of a HCVR that comprises the amino acid sequence set forth in SEQ ID NO: 277 (or a variant thereof); and a LCVR comprising the LCDR1, LCDR2 and LCDR3 of a LCVR that comprises the amino acid sequence set forth in SEQ ID NO: 282 (or a variant thereof); (8) a HCVR comprising the HCDR1, HCDR2 and HCDR3 of a HCVR that comprises the amino acid sequence set forth in SEQ ID NO: 287 (or a variant thereof); and a LCVR comprising the LCDR1, LCDR2 and LCDR3 of a LCVR that comprises the amino acid sequence set forth in SEQ ID NO: 292 (or a variant thereof); (9) a HCVR comprising the HCDR1, HCDR2 and HCDR3 of a HCVR that comprises the amino acid sequence set forth in SEQ ID NO: 297 (or a variant thereof); and a LCVR comprising the LCDR1, LCDR2 and LCDR3 of a LCVR that comprises the amino acid sequence set forth in SEQ ID NO: 302 (or a variant thereof); (10) a HCVR comprising the HCDR1, HCDR2 and HCDR3 of a HCVR that comprises the amino acid sequence set forth in SEQ ID NO: 307 (or a variant thereof); and a LCVR comprising the LCDR1, LCDR2 and LCDR3 of a LCVR that comprises the amino acid sequence set forth in SEQ ID NO: 312 (or a variant thereof); (11) a HCVR comprising the HCDR1, HCDR2 and HCDR3 of a HCVR that comprises the amino acid sequence set forth in SEQ ID NO: 317 (or a variant thereof); and a LCVR comprising the LCDR1, LCDR2 and LCDR3 of a LCVR that comprises the amino acid sequence set forth in SEQ ID NO: 322 (or a variant thereof); (12) a HCVR comprising the HCDR1, HCDR2 and HCDR3 of a HCVR that comprises the amino acid sequence set forth in SEQ ID NO: 327 (or a variant thereof); and a LCVR comprising the LCDR1, LCDR2 and LCDR3 of a LCVR that comprises the amino acid sequence set forth in SEQ ID NO: 332 (or a variant thereof); (13) a HCVR comprising the HCDR1, HCDR2 and HCDR3 of a HCVR that comprises the amino acid sequence set forth in SEQ ID NO: 337 (or a variant thereof); and a LCVR comprising the LCDR1, LCDR2 and LCDR3 of a LCVR that comprises the amino acid sequence set forth in SEQ ID NO: 342 (or a variant thereof); (14) a HCVR comprising the HCDR1, HCDR2 and HCDR3 of a HCVR that comprises the amino acid sequence set forth in SEQ ID NO: 347 (or a variant thereof); and a LCVR comprising the LCDR1, LCDR2 and LCDR3 of a LCVR that comprises the amino acid sequence set forth in SEQ ID NO: 352 (or a variant thereof); (15) a HCVR comprising the HCDR1, HCDR2 and HCDR3 of a HCVR that comprises the amino acid sequence set forth in SEQ ID NO: 357 (or a variant thereof); and a LCVR comprising the LCDR1, LCDR2 and LCDR3 of a LCVR that comprises the amino acid sequence set forth in SEQ ID NO: 362 (or a variant thereof); (16) a HCVR comprising the HCDR1, HCDR2 and HCDR3 of a HCVR that comprises the amino acid sequence set forth in SEQ ID NO: 367 (or a variant thereof); and a LCVR comprising the LCDR1, LCDR2 and LCDR3 of a LCVR that comprises the amino acid sequence set forth in SEQ ID NO: 372 (or a variant thereof); (17) a HCVR comprising the HCDR1, HCDR2 and HCDR3 of a HCVR that comprises the amino acid sequence set forth in SEQ ID NO: 377 (or a variant thereof); and a LCVR comprising the LCDR1, LCDR2 and LCDR3 of a LCVR that comprises the amino acid sequence set forth in SEQ ID NO: 382 (or a variant thereof); (18) a HCVR comprising the HCDR1, HCDR2 and HCDR3 of a HCVR that comprises the amino acid sequence set forth in SEQ ID NO: 387 (or a variant thereof); and a LCVR comprising the LCDR1, LCDR2 and LCDR3 of a LCVR that comprises the amino acid sequence set forth in SEQ ID NO: 392 (or a variant thereof); (19) a HCVR comprising the HCDR1, HCDR2 and HCDR3 of a HCVR that comprises the amino acid sequence set forth in SEQ ID NO: 397 (or a variant thereof); and a LCVR comprising the LCDR1, LCDR2 and LCDR3 of a LCVR that comprises the amino acid sequence set forth in SEQ ID NO: 402 (or a variant thereof); (20) a HCVR comprising the HCDR1, HCDR2 and HCDR3 of a HCVR that comprises the amino acid sequence set forth in SEQ ID NO: 407 (or a variant thereof); and a LCVR comprising the LCDR1, LCDR2 and LCDR3 of a LCVR that comprises the amino acid sequence set forth in SEQ ID NO: 412 (or a variant thereof); (21) a HCVR comprising the HCDR1, HCDR2 and HCDR3 of a HCVR that comprises the amino acid sequence set forth in SEQ ID NO: 417 (or a variant thereof); and a LCVR comprising the LCDR1, LCDR2 and LCDR3 of a LCVR that comprises the amino acid sequence set forth in SEQ ID NO: 422 (or a variant thereof); (22) a HCVR comprising the HCDR1, HCDR2 and HCDR3 of a HCVR that comprises the amino acid sequence set forth in SEQ ID NO: 427 (or a variant thereof); and a LCVR comprising the LCDR1, LCDR2 and LCDR3 of a LCVR that comprises the amino acid sequence set forth in SEQ ID NO: 432 (or a variant thereof); (23) a HCVR comprising the HCDR1, HCDR2 and HCDR3 of a HCVR that comprises the amino acid sequence set forth in SEQ ID NO: 437 (or a variant thereof); and a LCVR comprising the LCDR1, LCDR2 and LCDR3 of a LCVR that comprises the amino acid sequence set forth in SEQ ID NO: 442 (or a variant thereof); (24) a HCVR comprising the HCDR1, HCDR2 and HCDR3 of a HCVR that comprises the amino acid sequence set forth in SEQ ID NO: 447 (or a variant thereof); and a LCVR comprising the LCDR1, LCDR2 and LCDR3 of a LCVR that comprises the amino acid sequence set forth in SEQ ID NO: 452 (or a variant thereof); (25) a HCVR comprising the HCDR1, HCDR2 and HCDR3 of a HCVR that comprises the amino acid sequence set forth in SEQ ID NO: 457 (or a variant thereof); and a LCVR comprising the LCDR1, LCDR2 and LCDR3 of a LCVR that comprises the amino acid sequence set forth in SEQ ID NO: 462 (or a variant thereof); (26) a HCVR comprising the HCDR1, HCDR2 and HCDR3 of a HCVR that comprises the amino acid sequence set forth in SEQ ID NO: 467 (or a variant thereof); and a LCVR comprising the LCDR1, LCDR2 and LCDR3 of a LCVR that comprises the amino acid sequence set forth in SEQ ID NO: 472 (or a variant thereof); (27) a HCVR comprising the HCDR1, HCDR2 and HCDR3 of a HCVR that comprises the amino acid sequence set forth in SEQ ID NO: 477 (or a variant thereof); and a LCVR comprising the LCDR1, LCDR2 and LCDR3 of a LCVR that comprises the amino acid sequence set forth in SEQ ID NO: 482 (or a variant thereof); (28) a HCVR comprising the HCDR1, HCDR2 and HCDR3 of a HCVR that comprises the amino acid sequence set forth in SEQ ID NO: 487 (or a variant thereof); and a LCVR comprising the LCDR1, LCDR2 and LCDR3 of a LCVR that comprises the amino acid sequence set forth in SEQ ID NO: 492 (or a variant thereof); (29) a HCVR comprising the HCDR1, HCDR2 and HCDR3 of a HCVR that comprises the amino acid sequence set forth in SEQ ID NO: 497 (or a variant thereof); and a LCVR comprising the LCDR1, LCDR2 and LCDR3 of a LCVR that comprises the amino acid sequence set forth in SEQ ID NO: 502 (or a variant thereof); (30) a HCVR comprising the HCDR1, HCDR2 and HCDR3 of a HCVR that comprises the amino acid sequence set forth in SEQ ID NO: 507 (or a variant thereof); and a LCVR comprising the LCDR1, LCDR2 and LCDR3 of a LCVR that comprises the amino acid sequence set forth in SEQ ID NO: 512 (or a variant thereof); (31) a HCVR comprising the HCDR1, HCDR2 and HCDR3 of a HCVR that comprises the amino acid sequence set forth in SEQ ID NO: 517 (or a variant thereof); and a LCVR comprising the LCDR1, LCDR2 and LCDR3 of a LCVR that comprises the amino acid sequence set forth in SEQ ID NO: 522 (or a variant thereof); or (32) a HCVR comprising the HCDR1, HCDR2 and HCDR3 of a HCVR that comprises the amino acid sequence set forth in SEQ ID NO: 527 (or a variant thereof); and a LCVR comprising the LCDR1, LCDR2 and LCDR3 of a LCVR that comprises the amino acid sequence set forth in SEQ ID NO: 532 (or a variant thereof). In some such neonatal cells or populations of neonatal cells, the anti-TfR antigen binding protein comprises: (1) a HCVR comprising the HCDR1, HCDR2 and HCDR3 of a HCVR that comprises the amino acid sequence set forth in SEQ ID NO: 437 (or a variant thereof); and a LCVR comprising the LCDR1, LCDR2 and LCDR3 of a LCVR that comprises the amino acid sequence set forth in SEQ ID NO: 442 (or a variant thereof); or (2) a HCVR comprising the HCDR1, HCDR2 and HCDR3 of a HCVR that comprises the amino acid sequence set forth in SEQ ID NO: 457 (or a variant thereof); and a LCVR comprising the LCDR1, LCDR2 and LCDR3 of a LCVR that comprises the amino acid sequence set forth in SEQ ID NO: 462 (or a variant thereof). In some such neonatal cells or populations of neonatal cells, the anti-TfR antigen binding protein comprises: (a) a HCVR that comprises: an HCDR1 comprising the amino acid sequence set forth in SEQ ID NO: 218 (or a variant thereof), an HCDR2 comprising the amino acid sequence set forth in SEQ ID NO: 219 (or a variant thereof), and an HCDR3 comprising the amino acid sequence set forth in SEQ ID NO: 220 (or a variant thereof); and a LCVR that comprises: an LCDR1 comprising the amino acid sequence set forth in SEQ ID NO: 223 (or a variant thereof), an LCDR2 comprising the amino acid sequence set forth in SEQ ID NO: 224 (or a variant thereof), and an LCDR3 comprising the amino acid sequence set forth in SEQ ID NO: 225 (or a variant thereof); (b) a HCVR that comprises: an HCDR1 comprising the amino acid sequence set forth in SEQ ID NO: 228 (or a variant thereof), an HCDR2 comprising the amino acid sequence set forth in SEQ ID NO: 229 (or a variant thereof), and an HCDR3 comprising the amino acid sequence set forth in SEQ ID NO: 230 (or a variant thereof); and a LCVR that comprises: an LCDR1 comprising the amino acid sequence set forth in SEQ ID NO: 233 (or a variant thereof), an LCDR2 comprising the amino acid sequence set forth in SEQ ID NO: 234 (or a variant thereof), and an LCDR3 comprising the amino acid sequence set forth in SEQ ID NO: 235 (or a variant thereof); (c) a HCVR that comprises: an HCDR1 comprising the amino acid sequence set forth in SEQ ID NO: 238 (or a variant thereof), an HCDR2 comprising the amino acid sequence set forth in SEQ ID NO: 239 (or a variant thereof), and an HCDR3 comprising the amino acid sequence set forth in SEQ ID NO: 240 (or a variant thereof); and a LCVR that comprises: an LCDR1 comprising the amino acid sequence set forth in SEQ ID NO: 243 (or a variant thereof), an LCDR2 comprising the amino acid sequence set forth in SEQ ID NO: 244 (or a variant thereof), and an LCDR3 comprising the amino acid sequence set forth in SEQ ID NO: 245 (or a variant thereof); (d) a HCVR that comprises: an HCDR1 comprising the amino acid sequence set forth in SEQ ID NO: 248 (or a variant thereof), an HCDR2 comprising the amino acid sequence set forth in SEQ ID NO: 249 (or a variant thereof), and an HCDR3 comprising the amino acid sequence set forth in SEQ ID NO: 250 (or a variant thereof); and a LCVR that comprises: an LCDR1 comprising the amino acid sequence set forth in SEQ ID NO: 253 (or a variant thereof), an LCDR2 comprising the amino acid sequence set forth in SEQ ID NO: 254 (or a variant thereof), and an LCDR3 comprising the amino acid sequence set forth in SEQ ID NO: 255 (or a variant thereof); (e) a HCVR that comprises: an HCDR1 comprising the amino acid sequence set forth in SEQ ID NO: 258 (or a variant thereof), an HCDR2 comprising the amino acid sequence set forth in SEQ ID NO: 259 (or a variant thereof), and an HCDR3 comprising the amino acid sequence set forth in SEQ ID NO: 260 (or a variant thereof); and a LCVR that comprises: an LCDR1 comprising the amino acid sequence set forth in SEQ ID NO: 263 (or a variant thereof), an LCDR2 comprising the amino acid sequence set forth in SEQ ID NO: 264 (or a variant thereof), and an LCDR3 comprising the amino acid sequence set forth in SEQ ID NO: 265 (or a variant thereof); (f) a HCVR that comprises: an HCDR1 comprising the amino acid sequence set forth in SEQ ID NO: 268 (or a variant thereof), an HCDR2 comprising the amino acid sequence set forth in SEQ ID NO: 269 (or a variant thereof), and an HCDR3 comprising the amino acid sequence set forth in SEQ ID NO: 270 (or a variant thereof); and a LCVR that comprises: an LCDR1 comprising the amino acid sequence set forth in SEQ ID NO: 273 (or a variant thereof), an LCDR2 comprising the amino acid sequence set forth in SEQ ID NO: 274 (or a variant thereof), and an LCDR3 comprising the amino acid sequence set forth in SEQ ID NO: 275 (or a variant thereof); (g) a HCVR that comprises: an HCDR1 comprising the amino acid sequence set forth in SEQ ID NO: 278 (or a variant thereof), an HCDR2 comprising the amino acid sequence set forth in SEQ ID NO: 279 (or a variant thereof), and an HCDR3 comprising the amino acid sequence set forth in SEQ ID NO: 280 (or a variant thereof); and a LCVR that comprises: an LCDR1 comprising the amino acid sequence set forth in SEQ ID NO: 283 (or a variant thereof), an LCDR2 comprising the amino acid sequence set forth in SEQ ID NO: 284 (or a variant thereof), and an LCDR3 comprising the amino acid sequence set forth in SEQ ID NO: 285 (or a variant thereof); (h) a HCVR that comprises: an HCDR1 comprising the amino acid sequence set forth in SEQ ID NO: 288 (or a variant thereof), an HCDR2 comprising the amino acid sequence set forth in SEQ ID NO: 289 (or a variant thereof), and an HCDR3 comprising the amino acid sequence set forth in SEQ ID NO: 290 (or a variant thereof); and a LCVR that comprises: an LCDR1 comprising the amino acid sequence set forth in SEQ ID NO: 293 (or a variant thereof), an LCDR2 comprising the amino acid sequence set forth in SEQ ID NO: 294 (or a variant thereof), and an LCDR3 comprising the amino acid sequence set forth in SEQ ID NO: 295 (or a variant thereof); (i) a HCVR that comprises: an HCDR1 comprising the amino acid sequence set forth in SEQ ID NO: 298 (or a variant thereof), an HCDR2 comprising the amino acid sequence set forth in SEQ ID NO: 299 (or a variant thereof), and an HCDR3 comprising the amino acid sequence set forth in SEQ ID NO: 300 (or a variant thereof); and a LCVR that comprises: an LCDR1 comprising the amino acid sequence set forth in SEQ ID NO: 303 (or a variant thereof), an LCDR2 comprising the amino acid sequence set forth in SEQ ID NO: 304 (or a variant thereof), and an LCDR3 comprising the amino acid sequence set forth in SEQ ID NO: 305 (or a variant thereof); (j) a HCVR that comprises: an HCDR1 comprising the amino acid sequence set forth in SEQ ID NO: 308 (or a variant thereof), an HCDR2 comprising the amino acid sequence set forth in SEQ ID NO: 309 (or a variant thereof), and an HCDR3 comprising the amino acid sequence set forth in SEQ ID NO: 310 (or a variant thereof); and a LCVR that comprises: an LCDR1 comprising the amino acid sequence set forth in SEQ ID NO: 313 (or a variant thereof), an LCDR2 comprising the amino acid sequence set forth in SEQ ID NO: 314 (or a variant thereof), and an LCDR3 comprising the amino acid sequence set forth in SEQ ID NO: 315 (or a variant thereof); (k) a HCVR that comprises: an HCDR1 comprising the amino acid sequence set forth in SEQ ID NO: 318 (or a variant thereof), an HCDR2 comprising the amino acid sequence set forth in SEQ ID NO: 319 (or a variant thereof), and an HCDR3 comprising the amino acid sequence set forth in SEQ ID NO: 320 (or a variant thereof); and a LCVR that comprises: an LCDR1 comprising the amino acid sequence set forth in SEQ ID NO: 323 (or a variant thereof), an LCDR2 comprising the amino acid sequence set forth in SEQ ID NO: 324 (or a variant thereof), and an LCDR3 comprising the amino acid sequence set forth in SEQ ID NO: 325 (or a variant thereof); (1) a HCVR that comprises: an HCDR1 comprising the amino acid sequence set forth in SEQ ID NO: 328 (or a variant thereof), an HCDR2 comprising the amino acid sequence set forth in SEQ ID NO: 329 (or a variant thereof), and an HCDR3 comprising the amino acid sequence set forth in SEQ ID NO: 330 (or a variant thereof); and a LCVR that comprises: an LCDR1 comprising the amino acid sequence set forth in SEQ ID NO: 333 (or a variant thereof), an LCDR2 comprising the amino acid sequence set forth in SEQ ID NO: 334 (or a variant thereof), and an LCDR3 comprising the amino acid sequence set forth in SEQ ID NO: 335 (or a variant thereof); (m) a HCVR that comprises: an HCDR1 comprising the amino acid sequence set forth in SEQ ID NO: 338 (or a variant thereof), an HCDR2 comprising the amino acid sequence set forth in SEQ ID NO: 339 (or a variant thereof), and an HCDR3 comprising the amino acid sequence set forth in SEQ ID NO: 340 (or a variant thereof); and a LCVR that comprises: an LCDR1 comprising the amino acid sequence set forth in SEQ ID NO: 343 (or a variant thereof), an LCDR2 comprising the amino acid sequence set forth in SEQ ID NO: 344 (or a variant thereof), and an LCDR3 comprising the amino acid sequence set forth in SEQ ID NO: 345 (or a variant thereof); (n) a HCVR that comprises: an HCDR1 comprising the amino acid sequence set forth in SEQ ID NO: 348 (or a variant thereof), an HCDR2 comprising the amino acid sequence set forth in SEQ ID NO: 349 (or a variant thereof), and an HCDR3 comprising the amino acid sequence set forth in SEQ ID NO: 350 (or a variant thereof); and a LCVR that comprises: an LCDR1 comprising the amino acid sequence set forth in SEQ ID NO: 353 (or a variant thereof), an LCDR2 comprising the amino acid sequence set forth in SEQ ID NO: 354 (or a variant thereof), and an LCDR3 comprising the amino acid sequence set forth in SEQ ID NO: 355 (or a variant thereof); (o) a HCVR that comprises: an HCDR1 comprising the amino acid sequence set forth in SEQ ID NO: 358 (or a variant thereof), an HCDR2 comprising the amino acid sequence set forth in SEQ ID NO: 359 (or a variant thereof), and an HCDR3 comprising the amino acid sequence set forth in SEQ ID NO: 360 (or a variant thereof); and a LCVR that comprises: an LCDR1 comprising the amino acid sequence set forth in SEQ ID NO: 363 (or a variant thereof), an LCDR2 comprising the amino acid sequence set forth in SEQ ID NO: 364 (or a variant thereof), and an LCDR3 comprising the amino acid sequence set forth in SEQ ID NO: 365 (or a variant thereof); (p) a HCVR that comprises: an HCDR1 comprising the amino acid sequence set forth in SEQ ID NO: 368 (or a variant thereof), an HCDR2 comprising the amino acid sequence set forth in SEQ ID NO: 369 (or a variant thereof), and an HCDR3 comprising the amino acid sequence set forth in SEQ ID NO: 370 (or a variant thereof); and a LCVR that comprises: an LCDR1 comprising the amino acid sequence set forth in SEQ ID NO: 373 (or a variant thereof), an LCDR2 comprising the amino acid sequence set forth in SEQ ID NO: 374 (or a variant thereof), and an LCDR3 comprising the amino acid sequence set forth in SEQ ID NO: 375 (or a variant thereof); (q) a HCVR that comprises: an HCDR1 comprising the amino acid sequence set forth in SEQ ID NO: 378 (or a variant thereof), an HCDR2 comprising the amino acid sequence set forth in SEQ ID NO: 379 (or a variant thereof), and an HCDR3 comprising the amino acid sequence set forth in SEQ ID NO: 380 (or a variant thereof); and a LCVR that comprises: an LCDR1 comprising the amino acid sequence set forth in SEQ ID NO: 383 (or a variant thereof), an LCDR2 comprising the amino acid sequence set forth in SEQ ID NO: 384 (or a variant thereof), and an LCDR3 comprising the amino acid sequence set forth in SEQ ID NO: 385 (or a variant thereof); (r) a HCVR that comprises: an HCDR1 comprising the amino acid sequence set forth in SEQ ID NO: 388 (or a variant thereof), an HCDR2 comprising the amino acid sequence set forth in SEQ ID NO: 389 (or a variant thereof), and an HCDR3 comprising the amino acid sequence set forth in SEQ ID NO: 390 (or a variant thereof); and a LCVR that comprises: an LCDR1 comprising the amino acid sequence set forth in SEQ ID NO: 393 (or a variant thereof), an LCDR2 comprising the amino acid sequence set forth in SEQ ID NO: 394 (or a variant thereof), and an LCDR3 comprising the amino acid sequence set forth in SEQ ID NO: 395 (or a variant thereof); (s) a HCVR that comprises: an HCDR1 comprising the amino acid sequence set forth in SEQ ID NO: 398 (or a variant thereof), an HCDR2 comprising the amino acid sequence set forth in SEQ ID NO: 399 (or a variant thereof), and an HCDR3 comprising the amino acid sequence set forth in SEQ ID NO: 400 (or a variant thereof); and a LCVR that comprises: an LCDR1 comprising the amino acid sequence set forth in SEQ ID NO: 403 (or a variant thereof), an LCDR2 comprising the amino acid sequence set forth in SEQ ID NO: 404 (or a variant thereof), and an LCDR3 comprising the amino acid sequence set forth in SEQ ID NO: 405 (or a variant thereof); (t) a HCVR that comprises: an HCDR1 comprising the amino acid sequence set forth in SEQ ID NO: 408 (or a variant thereof), an HCDR2 comprising the amino acid sequence set forth in SEQ ID NO: 409 (or a variant thereof), and an HCDR3 comprising the amino acid sequence set forth in SEQ ID NO: 410 (or a variant thereof); and a LCVR that comprises: an LCDR1 comprising the amino acid sequence set forth in SEQ ID NO: 413 (or a variant thereof), an LCDR2 comprising the amino acid sequence set forth in SEQ ID NO: 414 (or a variant thereof), and an LCDR3 comprising the amino acid sequence set forth in SEQ ID NO: 415 (or a variant thereof); (u) a HCVR that comprises: an HCDR1 comprising the amino acid sequence set forth in SEQ ID NO: 418 (or a variant thereof), an HCDR2 comprising the amino acid sequence set forth in SEQ ID NO: 419 (or a variant thereof), and an HCDR3 comprising the amino acid sequence set forth in SEQ ID NO: 420 (or a variant thereof); and a LCVR that comprises: an LCDR1 comprising the amino acid sequence set forth in SEQ ID NO: 423 (or a variant thereof), an LCDR2 comprising the amino acid sequence set forth in SEQ ID NO: 424 (or a variant thereof), and an LCDR3 comprising the amino acid sequence set forth in SEQ ID NO: 425 (or a variant thereof); (v) a HCVR that comprises: an HCDR1 comprising the amino acid sequence set forth in SEQ ID NO: 428 (or a variant thereof), an HCDR2 comprising the amino acid sequence set forth in SEQ ID NO: 429 (or a variant thereof), and an HCDR3 comprising the amino acid sequence set forth in SEQ ID NO: 430 (or a variant thereof); and a LCVR that comprises: an LCDR1 comprising the amino acid sequence set forth in SEQ ID NO: 433 (or a variant thereof), an LCDR2 comprising the amino acid sequence set forth in SEQ ID NO: 434 (or a variant thereof), and an LCDR3 comprising the amino acid sequence set forth in SEQ ID NO: 435 (or a variant thereof); (w) a HCVR that comprises: an HCDR1 comprising the amino acid sequence set forth in SEQ ID NO: 438 (or a variant thereof), an HCDR2 comprising the amino acid sequence set forth in SEQ ID NO: 439 (or a variant thereof), and an HCDR3 comprising the amino acid sequence set forth in SEQ ID NO: 440 (or a variant thereof); and a LCVR that comprises: an LCDR1 comprising the amino acid sequence set forth in SEQ ID NO: 443 (or a variant thereof), an LCDR2 comprising the amino acid sequence set forth in SEQ ID NO: 444 (or a variant thereof), and an LCDR3 comprising the amino acid sequence set forth in SEQ ID NO: 445 (or a variant thereof); (x) a HCVR that comprises: an HCDR1 comprising the amino acid sequence set forth in SEQ ID NO: 448 (or a variant thereof), an HCDR2 comprising the amino acid sequence set forth in SEQ ID NO: 449 (or a variant thereof), and an HCDR3 comprising the amino acid sequence set forth in SEQ ID NO: 450 (or a variant thereof); and a LCVR that comprises: an LCDR1 comprising the amino acid sequence set forth in SEQ ID NO: 453 (or a variant thereof), an LCDR2 comprising the amino acid sequence set forth in SEQ ID NO: 454 (or a variant thereof), and an LCDR3 comprising the amino acid sequence set forth in SEQ ID NO: 455 (or a variant thereof); (y) a HCVR that comprises: an HCDR1 comprising the amino acid sequence set forth in SEQ ID NO: 458 (or a variant thereof), an HCDR2 comprising the amino acid sequence set forth in SEQ ID NO: 459 (or a variant thereof), and an HCDR3 comprising the amino acid sequence set forth in SEQ ID NO: 460 (or a variant thereof); and a LCVR that comprises: an LCDR1 comprising the amino acid sequence set forth in SEQ ID NO: 463 (or a variant thereof), an LCDR2 comprising the amino acid sequence set forth in SEQ ID NO: 464 (or a variant thereof), and an LCDR3 comprising the amino acid sequence set forth in SEQ ID NO: 465 (or a variant thereof); (z) a HCVR that comprises: an HCDR1 comprising the amino acid sequence set forth in SEQ ID NO: 468 (or a variant thereof), an HCDR2 comprising the amino acid sequence set forth in SEQ ID NO: 469 (or a variant thereof), and an HCDR3 comprising the amino acid sequence set forth in SEQ ID NO: 470 (or a variant thereof); and a LCVR that comprises: an LCDR1 comprising the amino acid sequence set forth in SEQ ID NO: 473 (or a variant thereof), an LCDR2 comprising the amino acid sequence set forth in SEQ ID NO: 474 (or a variant thereof), and an LCDR3 comprising the amino acid sequence set forth in SEQ ID NO: 475 (or a variant thereof); (aa) a HCVR that comprises: an HCDR1 comprising the amino acid sequence set forth in SEQ ID NO: 478 (or a variant thereof), an HCDR2 comprising the amino acid sequence set forth in SEQ ID NO: 479 (or a variant thereof), and an HCDR3 comprising the amino acid sequence set forth in SEQ ID NO: 480 (or a variant thereof); and a LCVR that comprises: an LCDR1 comprising the amino acid sequence set forth in SEQ ID NO: 483 (or a variant thereof), an LCDR2 comprising the amino acid sequence set forth in SEQ ID NO: 484 (or a variant thereof), and an LCDR3 comprising the amino acid sequence set forth in SEQ ID NO: 485 (or a variant thereof); (ab) a HCVR that comprises: an HCDR1 comprising the amino acid sequence set forth in SEQ ID NO: 488 (or a variant thereof), an HCDR2 comprising the amino acid sequence set forth in SEQ ID NO: 489 (or a variant thereof), and an HCDR3 comprising the amino acid sequence set forth in SEQ ID NO: 490 (or a variant thereof); and a LCVR that comprises: an LCDR1 comprising the amino acid sequence set forth in SEQ ID NO: 493 (or a variant thereof), an LCDR2 comprising the amino acid sequence set forth in SEQ ID NO: 494 (or a variant thereof), and an LCDR3 comprising the amino acid sequence set forth in SEQ ID NO: 495 (or a variant thereof); (ac) a HCVR that comprises: an HCDR1 comprising the amino acid sequence set forth in SEQ ID NO: 498 (or a variant thereof), an HCDR2 comprising the amino acid sequence set forth in SEQ ID NO: 499 (or a variant thereof), and an HCDR3 comprising the amino acid sequence set forth in SEQ ID NO: 500 (or a variant thereof); and a LCVR that comprises: an LCDR1 comprising the amino acid sequence set forth in SEQ ID NO: 503 (or a variant thereof), an LCDR2 comprising the amino acid sequence set forth in SEQ ID NO: 504 (or a variant thereof), and an LCDR3 comprising the amino acid sequence set forth in SEQ ID NO: 505 (or a variant thereof); (ad) a HCVR that comprises: an HCDR1 comprising the amino acid sequence set forth in SEQ ID NO: 508 (or a variant thereof), an HCDR2 comprising the amino acid sequence set forth in SEQ ID NO: 509 (or a variant thereof), and an HCDR3 comprising the amino acid sequence set forth in SEQ ID NO: 510 (or a variant thereof); and a LCVR that comprises: an LCDR1 comprising the amino acid sequence set forth in SEQ ID NO: 513 (or a variant thereof), an LCDR2 comprising the amino acid sequence set forth in SEQ ID NO: 514 (or a variant thereof), and an LCDR3 comprising the amino acid sequence set forth in SEQ ID NO: 515 (or a variant thereof); (ae) a HCVR that comprises: an HCDR1 comprising the amino acid sequence set forth in SEQ ID NO: 518 (or a variant thereof), an HCDR2 comprising the amino acid sequence set forth in SEQ ID NO: 519 (or a variant thereof), and an HCDR3 comprising the amino acid sequence set forth in SEQ ID NO: 520 (or a variant thereof); and a LCVR that comprises: an LCDR1 comprising the amino acid sequence set forth in SEQ ID NO: 523 (or a variant thereof), an LCDR2 comprising the amino acid sequence set forth in SEQ ID NO: 524 (or a variant thereof), and an LCDR3 comprising the amino acid sequence set forth in SEQ ID NO: 525 (or a variant thereof); and/or (af) a HCVR that comprises: an HCDR1 comprising the amino acid sequence set forth in SEQ ID NO: 528 (or a variant thereof), an HCDR2 comprising the amino acid sequence set forth in SEQ ID NO: 529 (or a variant thereof), and an HCDR3 comprising the amino acid sequence set forth in SEQ ID NO: 530 (or a variant thereof); and a LCVR that comprises: an LCDR1 comprising the amino acid sequence set forth in SEQ ID NO: 533 (or a variant thereof), an LCDR2 comprising the amino acid sequence set forth in SEQ ID NO: 534 (or a variant thereof), and an LCDR3 comprising the amino acid sequence set forth in SEQ ID NO: 535 (or a variant thereof). In some such neonatal cells or populations of neonatal cells, the anti-TfR antigen binding protein comprises: (a) a HCVR that comprises: an HCDR1 comprising the amino acid sequence set forth in SEQ ID NO: 438 (or a variant thereof), an HCDR2 comprising the amino acid sequence set forth in SEQ ID NO: 439 (or a variant thereof), and an HCDR3 comprising the amino acid sequence set forth in SEQ ID NO: 440 (or a variant thereof); and a LCVR that comprises: an LCDR1 comprising the amino acid sequence set forth in SEQ ID NO: 443 (or a variant thereof), an LCDR2 comprising the amino acid sequence set forth in SEQ ID NO: 444 (or a variant thereof), and an LCDR3 comprising the amino acid sequence set forth in SEQ ID NO: 445 (or a variant thereof); or (b) a HCVR that comprises: an HCDR1 comprising the amino acid sequence set forth in SEQ ID NO: 458 (or a variant thereof), an HCDR2 comprising the amino acid sequence set forth in SEQ ID NO: 459 (or a variant thereof), and an HCDR3 comprising the amino acid sequence set forth in SEQ ID NO: 460 (or a variant thereof); and a LCVR that comprises: an LCDR1 comprising the amino acid sequence set forth in SEQ ID NO: 463 (or a variant thereof), an LCDR2 comprising the amino acid sequence set forth in SEQ ID NO: 464 (or a variant thereof), and an LCDR3 comprising the amino acid sequence set forth in SEQ ID NO: 465 (or a variant thereof). In some such neonatal cells or populations of neonatal cells, the anti-TfR antigen binding protein comprises: (i) a HCVR that comprises the amino acid sequence set forth in SEQ ID NO: 217 (or a variant thereof); and a LCVR that comprises the amino acid sequence set forth in SEQ ID NO: 222 (or a variant thereof); (ii) a HCVR that comprises the amino acid sequence set forth in SEQ ID NO: 227 (or a variant thereof); and a LCVR that comprises the amino acid sequence set forth in SEQ ID NO: 232 (or a variant thereof); (iii) a HCVR that comprises the amino acid sequence set forth in SEQ ID NO: 237 (or a variant thereof); and a LCVR that comprises the amino acid sequence set forth in SEQ ID NO: 242 (or a variant thereof); (iv) a HCVR that comprises the amino acid sequence set forth in SEQ ID NO: 247 (or a variant thereof); and a LCVR that comprises the amino acid sequence set forth in SEQ ID NO: 252 (or a variant thereof); (v) a HCVR that comprises the amino acid sequence set forth in SEQ ID NO: 257 (or a variant thereof); and a LCVR that comprises the amino acid sequence set forth in SEQ ID NO: 262 (or a variant thereof); (vi) a HCVR that comprises the amino acid sequence set forth in SEQ ID NO: 267 (or a variant thereof); and a LCVR that comprises the amino acid sequence set forth in SEQ ID NO: 272 (or a variant thereof); (vii) a HCVR that comprises the amino acid sequence set forth in SEQ ID NO: 277 (or a variant thereof); and a LCVR that comprises the amino acid sequence set forth in SEQ ID NO: 282 (or a variant thereof); (viii) a HCVR that comprises the amino acid sequence set forth in SEQ ID NO: 287 (or a variant thereof); and a LCVR that comprises the amino acid sequence set forth in SEQ ID NO: 292 (or a variant thereof); (ix) a HCVR that comprises the amino acid sequence set forth in SEQ ID NO: 297 (or a variant thereof); and a LCVR that comprises the amino acid sequence set forth in SEQ ID NO: 302 (or a variant thereof); (x) a HCVR that comprises the amino acid sequence set forth in SEQ ID NO: 307 (or a variant thereof); and a LCVR that comprises the amino acid sequence set forth in SEQ ID NO: 312 (or a variant thereof); (xi) a HCVR that comprises the amino acid sequence set forth in SEQ ID NO: 317 (or a variant thereof); and a LCVR that comprises the amino acid sequence set forth in SEQ ID NO: 322 (or a variant thereof); (xii) a HCVR that comprises the amino acid sequence set forth in SEQ ID NO: 327 (or a variant thereof); and a LCVR that comprises the amino acid sequence set forth in SEQ ID NO: 332 (or a variant thereof); (xiii) a HCVR that comprises the amino acid sequence set forth in SEQ ID NO: 337 (or a variant thereof); and a LCVR that comprises the amino acid sequence set forth in SEQ ID NO: 342 (or a variant thereof); (xiv) a HCVR that comprises the amino acid sequence set forth in SEQ ID NO: 347 (or a variant thereof); and a LCVR that comprises the amino acid sequence set forth in SEQ ID NO: 352 (or a variant thereof); (xv) a HCVR that comprises the amino acid sequence set forth in SEQ ID NO: 357 (or a variant thereof); and a LCVR that comprises the amino acid sequence set forth in SEQ ID NO: 362 (or a variant thereof); (xvi) a HCVR that comprises the amino acid sequence set forth in SEQ ID NO: 367 (or a variant thereof); and a LCVR that comprises the amino acid sequence set forth in SEQ ID NO: 372 (or a variant thereof); (xvii) a HCVR that comprises the amino acid sequence set forth in SEQ ID NO: 377 (or a variant thereof); and a LCVR that comprises the amino acid sequence set forth in SEQ ID NO: 382 (or a variant thereof); (xviii) a HCVR that comprises the amino acid sequence set forth in SEQ ID NO: 387 (or a variant thereof); and a LCVR that comprises the amino acid sequence set forth in SEQ ID NO: 392 (or a variant thereof); (xix) a HCVR that comprises the amino acid sequence set forth in SEQ ID NO: 397 (or a variant thereof); and a LCVR that comprises the amino acid sequence set forth in SEQ ID NO: 402 (or a variant thereof); (xx) a HCVR that comprises the amino acid sequence set forth in SEQ ID NO: 407 (or a variant thereof); and a LCVR that comprises the amino acid sequence set forth in SEQ ID NO: 412 (or a variant thereof); (xxi) a HCVR that comprises the amino acid sequence set forth in SEQ ID NO: 417 (or a variant thereof); and a LCVR that comprises the amino acid sequence set forth in SEQ ID NO: 422 (or a variant thereof); (xxii) a HCVR that comprises the amino acid sequence set forth in SEQ ID NO: 427 (or a variant thereof); and a LCVR that comprises the amino acid sequence set forth in SEQ ID NO: 432 (or a variant thereof); (xxiii) a HCVR that comprises the amino acid sequence set forth in SEQ ID NO: 437 (or a variant thereof); and a LCVR that comprises the amino acid sequence set forth in SEQ ID NO: 442 (or a variant thereof); (xxiv) a HCVR that comprises the amino acid sequence set forth in SEQ ID NO: 447 (or a variant thereof); and a LCVR that comprises the amino acid sequence set forth in SEQ ID NO: 452 (or a variant thereof); (xxv) a HCVR that comprises the amino acid sequence set forth in SEQ ID NO: 457 (or a variant thereof); and a LCVR that comprises the amino acid sequence set forth in SEQ ID NO: 462 (or a variant thereof); (xxvi) a HCVR that comprises the amino acid sequence set forth in SEQ ID NO: 467 (or a variant thereof); and a LCVR that comprises the amino acid sequence set forth in SEQ ID NO: 472 (or a variant thereof); (xxvii) a HCVR that comprises the amino acid sequence set forth in SEQ ID NO: 477 (or a variant thereof); and a LCVR that comprises the amino acid sequence set forth in SEQ ID NO: 482 (or a variant thereof); (xxviii) a HCVR that comprises the amino acid sequence set forth in SEQ ID NO: 487 (or a variant thereof); and a LCVR that comprises the amino acid sequence set forth in SEQ ID NO: 492 (or a variant thereof); (xxix) a HCVR that comprises the amino acid sequence set forth in SEQ ID NO: 497 (or a variant thereof); and a LCVR that comprises the amino acid sequence set forth in SEQ ID NO: 502 (or a variant thereof); (xxx) a HCVR that comprises the amino acid sequence set forth in SEQ ID NO: 507 (or a variant thereof); and a LCVR that comprises the amino acid sequence set forth in SEQ ID NO: 512 (or a variant thereof); (xxxi) a HCVR that comprises the amino acid sequence set forth in SEQ ID NO: 517 (or a variant thereof); and a LCVR that comprises the amino acid sequence set forth in SEQ ID NO: 522 (or a variant thereof); and/or (xxxii) a HCVR that comprises the amino acid sequence set forth in SEQ ID NO: 527 (or a variant thereof); and a LCVR that comprises the amino acid sequence set forth in SEQ ID NO: 532 (or a variant thereof). In some such neonatal cells or populations of neonatal cells, the anti-TfR antigen binding protein comprises: (i) a HCVR that comprises the amino acid sequence set forth in SEQ ID NO: 437 (or a variant thereof); and a LCVR that comprises the amino acid sequence set forth in SEQ ID NO: 442 (or a variant thereof); or (ii) a HCVR that comprises the amino acid sequence set forth in SEQ ID NO: 457 (or a variant thereof); and a LCVR that comprises the amino acid sequence set forth in SEQ ID NO: 462 (or a variant thereof). In some such neonatal cells or populations of neonatal cells, the TfR-binding delivery domain comprises an anti-TfR antibody, antibody fragment, or single-chain variable fragment (scFv). In some such neonatal cells or populations of neonatal cells, the TfR-binding delivery domain is the single-chain variable fragment (scFv), optionally wherein the multidomain therapeutic protein comprises domains arranged in the following orientation: N′-Heavy chain variable region-Light chain variable region-lysosomal alpha-glucosidase-C′ or N′-Light chain variable region-Heavy chain variable region-lysosomal alpha-glucosidase-C′, optionally wherein the scFv and lysosomal alpha-glucosidase are connected by a peptide linker, and optionally wherein the peptide linker which is -(GGGGS)ₘ- (SEQ ID NO: 600); wherein m is 1, 2, 3, 4, 5, 6, 7, 8, 9, or 10, optionally wherein the scFv variable regions are connected by a peptide linker, and optionally wherein the peptide linker which is -(GGGGS)ₘ- (SEQ ID NO: 600); wherein m is 1, 2, 3, 4, 5, 6, 7, 8, 9, or 10. In some such neonatal cells or populations of neonatal cells, the multidomain therapeutic protein comprises a heavy chain variable region (V_{H}) and a light chain variable region (V_{L}), and a lysosomal alpha-glucosidase, wherein the V_{H}, V_{L} and lysosomal alpha-glucosidase are arranged as follows: (i) V_{L}-V_{H}-lysosomal alpha-glucosidase; (ii) V_{H}-V_{L}-lysosomal alpha-glucosidase; (iii) V_{L}-[(GGGGS)_{3]}-V_{H}-[(GGGGS)_{2]}-lysosomal alpha-glucosidase; or (iv) V_{H}-[(GGGGS)₃]-V_{L}-[(GGGGS)₂]-lysosomal alpha-glucosidase. In some such neonatal cells or populations of neonatal cells, the scFv comprises the sequence set forth in any one of SEQ ID NOS: 540, 549, 551, and 554, optionally wherein the scFv comprises the sequence set forth in SEQ ID NO: 554. In some such neonatal cells or populations of neonatal cells, the scFv consists of the sequence set forth in any one of SEQ ID NOS: 540, 549, 551, and 554, optionally wherein the scFv consists of the sequence set forth in SEQ ID NOS: 554. In some such neonatal cells or populations of neonatal cells, the scFv coding sequence is at least 90%, at least 91%, at least 92%, at least 93%, at least 94%, at least 95%, at least 96%, at least 97%, at least 98%, or at least 99% identical to any one of SEQ ID NOS: 587-599. Optionally, the scFv coding sequence is at least 90%, at least 91%, at least 92%, at least 93%, at least 94%, at least 95%, at least 96%, at least 97%, at least 98%, or at least 99% identical to any one of SEQ ID NOS: 593-595, optionally wherein the scFv coding sequence is at least 90%, at least 91%, at least 92%, at least 93%, at least 94%, at least 95%, at least 96%, at least 97%, at least 98%, or at least 99% identical to SEQ ID NO: 593. Optionally, the scFv coding sequence is at least 90%, at least 91%, at least 92%, at least 93%, at least 94%, at least 95%, at least 96%, at least 97%, at least 98%, or at least 99% identical to any one of SEQ ID NOS: 590-592, optionally wherein the scFv coding sequence is at least 90%, at least 91%, at least 92%, at least 93%, at least 94%, at least 95%, at least 96%, at least 97%, at least 98%, or at least 99% identical to SEQ ID NO: 590. In some such neonatal cells or populations of neonatal cells, the scFv coding sequence is at least 90%, at least 91%, at least 92%, at least 93%, at least 94%, at least 95%, at least 96%, at least 97%, at least 98%, or at least 99% identical to any one of SEQ ID NOS: 587-599 and encodes an scFv comprising any one of SEQ ID NOS: 540, 549, 551, and 554. Optionally, the scFv coding sequence is at least 90%, at least 91%, at least 92%, at least 93%, at least 94%, at least 95%, at least 96%, at least 97%, at least 98%, or at least 99% identical to any one of SEQ ID NOS: 593-595 and encodes an scFv comprising SEQ ID NO: 554, optionally wherein the scFv coding sequence is at least 90%, at least 91%, at least 92%, at least 93%, at least 94%, at least 95%, at least 96%, at least 97%, at least 98%, or at least 99% identical to SEQ ID NO: 593 and encodes an scFv comprising SEQ ID NO: 554. Optionally, the scFv coding sequence is at least 90%, at least 91%, at least 92%, at least 93%, at least 94%, at least 95%, at least 96%, at least 97%, at least 98%, or at least 99% identical to any one of SEQ ID NOS: 590-592 and encodes an scFv comprising SEQ ID NO: 551, optionally wherein the scFv coding sequence is at least 90%, at least 91%, at least 92%, at least 93%, at least 94%, at least 95%, at least 96%, at least 97%, at least 98%, or at least 99% identical to SEQ ID NO: 590 and encodes an scFv comprising SEQ ID NO: 551. In some such neonatal cells or populations of neonatal cells, the scFv coding sequence is at least 90%, at least 91%, at least 92%, at least 93%, at least 94%, at least 95%, at least 96%, at least 97%, at least 98%, or at least 99% identical to any one of SEQ ID NOS: 587-599, is codon-optimized and CpG-depleted, and encodes an scFv comprising any one of SEQ ID NOS: 540, 549, 551, and 554. Optionally, the scFv coding sequence is at least 90%, at least 91%, at least 92%, at least 93%, at least 94%, at least 95%, at least 96%, at least 97%, at least 98%, or at least 99% identical to any one of SEQ ID NOS: 593-595, is codon-optimized and CpG-depleted, and encodes an scFv comprising SEQ ID NO: 554, optionally wherein the scFv coding sequence is at least 90%, at least 91%, at least 92%, at least 93%, at least 94%, at least 95%, at least 96%, at least 97%, at least 98%, or at least 99% identical to SEQ ID NO: 593, the scFv coding sequence is codon-optimized and CpG-depleted, and encodes an scFv comprising SEQ ID NO: 554. Optionally, the scFv coding sequence is at least 90%, at least 91%, at least 92%, at least 93%, at least 94%, at least 95%, at least 96%, at least 97%, at least 98%, or at least 99% identical to any one of SEQ ID NOS: 590-592, is codon-optimized and CpG-depleted, and encodes an scFv comprising SEQ ID NO: 551, optionally wherein the scFv coding sequence is at least 90%, at least 91%, at least 92%, at least 93%, at least 94%, at least 95%, at least 96%, at least 97%, at least 98%, or at least 99% identical to SEQ ID NO: 590, the scFv coding sequence is codon-optimized and CpG-depleted, and encodes an scFv comprising SEQ ID NO: 551. In some such neonatal cells or populations of neonatal cells, the scFv coding sequence comprises the sequence set forth in any one of SEQ ID NOS: 587-599. Optionally, the scFv coding sequence comprises the sequence set forth in any one of SEQ ID NOS: 593-595, optionally wherein the scFv coding sequence comprises the sequence set forth in SEQ ID NO: 593. Optionally, the scFv coding sequence comprises the sequence set forth in any one of SEQ ID NOS: 590-592, optionally wherein the scFv coding sequence comprises the sequence set forth in SEQ ID NO: 590. In some such neonatal cells or populations of neonatal cells, the scFv coding sequence consists of the sequence set forth in any one of SEQ ID NOS: 587-599. Optionally, the scFv coding sequence consists of the sequence set forth in any one of SEQ ID NOS: 593-595, optionally wherein the scFv coding sequence consists of sequence set forth in SEQ ID NO: 593. Optionally, the scFv coding sequence consists of the sequence set forth in any one of SEQ ID NOS: 590-592, optionally wherein the scFv coding sequence consists of sequence set forth in SEQ ID NO: 590.

In some such neonatal cells or populations of neonatal cells, the lysosomal alpha-glucosidase lacks the lysosomal alpha-glucosidase signal peptide and propeptide. In some such neonatal cells or populations of neonatal cells, the lysosomal alpha-glucosidase comprises the sequence set forth in SEQ ID NO: 173. In some such neonatal cells or populations of neonatal cells, the lysosomal alpha-glucosidase consists of the sequence set forth in SEQ ID NO: 173. In some such neonatal cells or populations of neonatal cells, the lysosomal alpha-glucosidase coding sequence is codon-optimized or CpG-depleted. In some such neonatal cells or populations of neonatal cells, the lysosomal alpha-glucosidase coding sequence is codon-optimized and CpG-depleted. In some such neonatal cells or populations of neonatal cells, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and 205-212, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176. In some such neonatal cells or populations of neonatal cells, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and 205-212 and encodes a lysosomal alpha-glucosidase protein comprising SEQ ID NO: 173, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176 and encodes a lysosomal alpha-glucosidase protein comprising SEQ ID NO: 173. In some such neonatal cells or populations of neonatal cells, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and 205-212, is codon-optimized and CpG-depleted, and encodes a lysosomal alpha-glucosidase protein comprising SEQ ID NO: 173, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176, is codon-optimized and CpG-depleted, and encodes a lysosomal alpha-glucosidase protein comprising SEQ ID NO: 173. In some such neonatal cells or populations of neonatal cells, the lysosomal alpha-glucosidase coding sequence comprises the sequence set forth in any one of SEQ ID NOS: 174-182 and 205-212, optionally wherein the lysosomal alpha-glucosidase coding sequence comprises the sequence set forth in SEQ ID NO: 176. In some such neonatal cells or populations of neonatal cells, the lysosomal alpha-glucosidase coding sequence consists of the sequence set forth in any one of SEQ ID NOS: 174-182 and 205-212, optionally wherein the lysosomal alpha-glucosidase coding sequence consists of the sequence set forth in SEQ ID NO: 176.

In some such neonatal cells or populations of neonatal cells, the lysosomal alpha-glucosidase lacks the lysosomal alpha-glucosidase signal peptide and propeptide. In some such neonatal cells or populations of neonatal cells, the lysosomal alpha-glucosidase comprises the sequence set forth in SEQ ID NO: 173. In some such neonatal cells or populations of neonatal cells, the lysosomal alpha-glucosidase consists of the sequence set forth in SEQ ID NO: 173. In some such neonatal cells or populations of neonatal cells, the lysosomal alpha-glucosidase coding sequence is codon-optimized or CpG-depleted. In some such neonatal cells or populations of neonatal cells, the lysosomal alpha-glucosidase coding sequence is codon-optimized and CpG-depleted. In some such neonatal cells or populations of neonatal cells, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176. In some such neonatal cells or populations of neonatal cells, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and encodes a lysosomal alpha-glucosidase protein comprising SEQ ID NO: 173, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176 and encodes a lysosomal alpha-glucosidase protein comprising SEQ ID NO: 173. In some such neonatal cells or populations of neonatal cells, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182, is codon-optimized and CpG-depleted, and encodes a lysosomal alpha-glucosidase protein comprising SEQ ID NO: 173, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176, is codon-optimized and CpG-depleted, and encodes a lysosomal alpha-glucosidase protein comprising SEQ ID NO: 173. In some such neonatal cells or populations of neonatal cells, the lysosomal alpha-glucosidase coding sequence comprises the sequence set forth in any one of SEQ ID NOS: 174-182, optionally wherein the lysosomal alpha-glucosidase coding sequence comprises the sequence set forth in SEQ ID NO: 176. In some such neonatal cells or populations of neonatal cells, the lysosomal alpha-glucosidase coding sequence consists of the sequence set forth in any one of SEQ ID NOS: 174-182, optionally wherein the lysosomal alpha-glucosidase coding sequence consists of the sequence set forth in SEQ ID NO: 176.

In some such neonatal cells or populations of neonatal cells, the coding sequence for the multidomain therapeutic protein is codon-optimized or CpG-depleted. In some such neonatal cells or populations of neonatal cells, the coding sequence for the multidomain therapeutic protein is codon-optimized and CpG-depleted. In some such neonatal cells or populations of neonatal cells, the multidomain therapeutic protein comprises the sequence set forth in any one of SEQ ID NOS: 570-573, optionally wherein the multidomain therapeutic protein comprises the sequence set forth in SEQ ID NO: 573. In some such neonatal cells or populations of neonatal cells, the multidomain therapeutic protein consists of the sequence set forth in any one of SEQ ID NOS: 570-573, optionally wherein the multidomain therapeutic protein consists of the sequence set forth in SEQ ID NO: 573. In some such neonatal cells or populations of neonatal cells, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 574-586. Optionally, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 584-586, optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 584, optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 733. Optionally, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 581-583, optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 581, optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 729. In some such neonatal cells or populations of neonatal cells, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 574-586, and the multidomain therapeutic protein comprises the sequence set forth in any one of SEQ ID NOS: 570-573. Optionally, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 584-586, and the multidomain therapeutic protein comprises the sequence set forth in SEQ ID NO: 573, optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 584, and the multidomain therapeutic protein comprises the sequence set forth in SEQ ID NO: 573, optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 733, and the multidomain therapeutic protein comprises the sequence set forth in SEQ ID NO: 573. Optionally, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 581-583, and the multidomain therapeutic protein comprises the sequence set forth in SEQ ID NO: 572, optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 581, and the multidomain therapeutic protein comprises the sequence set forth in SEQ ID NO: 572, optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 729, and the multidomain therapeutic protein comprises the sequence set forth in SEQ ID NO: 572. In some such neonatal cells or populations of neonatal cells, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 574-586 and is codon-optimized and CpG-depleted, and the multidomain therapeutic protein comprises the sequence set forth in any one of SEQ ID NOS: 570-573. Optionally, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 584-586 and is codon-optimized and CpG-depleted, and the multidomain therapeutic protein comprises the sequence set forth in SEQ ID NO: 573, optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 584, the coding sequence for the multidomain therapeutic protein is codon-optimized and CpG-depleted, and the multidomain therapeutic protein comprises the sequence set forth in SEQ ID NO: 573, optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 733, the coding sequence for the multidomain therapeutic protein is codon-optimized and CpG-depleted, and the multidomain therapeutic protein comprises the sequence set forth in SEQ ID NO: 573. Optionally, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 581-583 and is codon-optimized and CpG-depleted, and the multidomain therapeutic protein comprises the sequence set forth in SEQ ID NO: 572, optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 581, the coding sequence for the multidomain therapeutic protein is codon-optimized and CpG-depleted, and the multidomain therapeutic protein comprises the sequence set forth in SEQ ID NO: 572, optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 729, the coding sequence for the multidomain therapeutic protein is codon-optimized and CpG-depleted, and the multidomain therapeutic protein comprises the sequence set forth in SEQ ID NO: 572. In some such neonatal cells or populations of neonatal cells, the coding sequence for the multidomain therapeutic protein comprises the sequence set forth in any one of SEQ ID NOS: 574-586. Optionally, the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733. Optionally, the coding sequence for the multidomain therapeutic protein comprises the sequence set forth in any one of SEQ ID NOS: 581-583, optionally wherein the coding sequence for the multidomain therapeutic protein comprises the sequence set forth in SEQ ID NO: 581, optionally wherein the nucleic acid construct comprises the sequence set forth in SEQ ID NO: 729. In some such neonatal cells or populations of neonatal cells, the coding sequence for the multidomain therapeutic protein consists of the sequence set forth in any one of SEQ ID NOS: 574-586. Optionally, the coding sequence for the multidomain therapeutic protein consists of the sequence set forth in any one of SEQ ID NOS: 584-586, optionally wherein the coding sequence for the multidomain therapeutic protein consists of the sequence set forth in SEQ ID NO: 584, optionally wherein the nucleic acid construct comprises the sequence set forth in SEQ ID NO: 733. Optionally, the coding sequence for the multidomain therapeutic protein consists of the sequence set forth in any one of SEQ ID NOS: 581-583, optionally wherein the coding sequence for the multidomain therapeutic protein consists of the sequence set forth in SEQ ID NO: 581, optionally wherein the nucleic acid construct comprises the sequence set forth in SEQ ID NO: 729. In some such neonatal cells or populations of neonatal cells, the nucleic acid construct comprises a splice acceptor upstream of the coding sequence for the multidomain therapeutic protein. In some such neonatal cells or populations of neonatal cells, the nucleic acid construct comprises a polyadenylation signal or sequence downstream of the coding sequence for the multidomain therapeutic protein. In some such neonatal cells or populations of neonatal cells, the nucleic acid construct comprises a splice acceptor upstream of the coding sequence for the multidomain therapeutic protein, and the nucleic acid construct comprises a polyadenylation signal or sequence downstream of the coding sequence for the multidomain therapeutic protein. In some such neonatal cells or populations of neonatal cells, the nucleic acid construct does not comprise a promoter that drives the expression of the multidomain therapeutic protein, and wherein the coding sequence for the multidomain therapeutic protein is operably linked to an endogenous promoter at the target genomic locus. In some such neonatal cells or populations of neonatal cells, the coding sequence for the multidomain therapeutic protein in the nucleic acid construct is operably linked to a promoter, optionally wherein the promoter is a liver-specific promoter. In some such neonatal cells or populations of neonatal cells, the nucleic acid construct comprises from 5′ to 3′: a splice acceptor, the coding sequence for the multidomain therapeutic protein, and a polyadenylation signal or sequence, wherein the lysosomal alpha-glucosidase coding sequence comprises the sequence set forth in any one of SEQ ID NOS: 174-182 and 205-212, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733, or optionally wherein the coding sequence for the multidomain therapeutic protein comprises the sequence set forth in any one of SEQ ID NOS: 581-583, optionally wherein the coding sequence for the multidomain therapeutic protein comprises the sequence set forth in SEQ ID NO: 581, optionally wherein the nucleic acid construct comprises the sequence set forth in SEQ ID NO: 729, wherein the nucleic acid construct does not comprise a promoter that drives the expression of the multidomain therapeutic protein, and wherein the coding sequence for the multidomain therapeutic protein is operably linked to an endogenous promoter at the target genomic locus. In some such neonatal cells or populations of neonatal cells, the nucleic acid construct comprises from 5′ to 3′: a splice acceptor, the coding sequence for the multidomain therapeutic protein, and a polyadenylation signal or sequence, wherein the lysosomal alpha-glucosidase coding sequence comprises the sequence set forth in any one of SEQ ID NOS: 174-182, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733, or optionally wherein the coding sequence for the multidomain therapeutic protein comprises the sequence set forth in any one of SEQ ID NOS: 581-583, optionally wherein the coding sequence for the multidomain therapeutic protein comprises the sequence set forth in SEQ ID NO: 581, optionally wherein the nucleic acid construct comprises the sequence set forth in SEQ ID NO: 729, wherein the nucleic acid construct does not comprise a promoter that drives the expression of the multidomain therapeutic protein, and wherein the coding sequence for the multidomain therapeutic protein is operably linked to an endogenous promoter at the target genomic locus.

In some such neonatal cells or populations of neonatal cells, the target genomic locus is an albumin gene, optionally wherein the albumin gene is a human albumin gene. In some such neonatal cells or populations of neonatal cells, the nuclease target site is in intron 1 of the albumin gene.

In another aspect, provided are compositions comprising a nucleic acid construct comprising a coding sequence for a multidomain therapeutic protein comprising a TfR-binding delivery domain fused to a lysosomal alpha-glucosidase, wherein the lysosomal alpha-glucosidase coding sequence is CpG-depleted relative to a wild type lysosomal alpha-glucosidase coding sequence. In some such compositions, the TfR-binding delivery domain is fused to the lysosomal alpha-glucosidase protein via a peptide linker. In some such compositions, the lysosomal alpha-glucosidase lacks the lysosomal alpha-glucosidase signal peptide and propeptide. In some such compositions, the lysosomal alpha-glucosidase comprises the sequence set forth in SEQ ID NO: 173. In some such compositions, the lysosomal alpha-glucosidase consists of the sequence set forth in SEQ ID NO: 173. In some such compositions, the lysosomal alpha-glucosidase coding sequence is codon-optimized and CpG-depleted. In some such compositions, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and 205-212, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176. In some such compositions, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and 205-212 and encodes a lysosomal alpha-glucosidase protein comprising SEQ ID NO: 173, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176 and encodes a lysosomal alpha-glucosidase protein comprising SEQ ID NO: 173. In some such compositions, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and 205-212, is codon-optimized and CpG-depleted, and encodes a lysosomal alpha-glucosidase protein comprising SEQ ID NO: 173, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176, is codon-optimized and CpG-depleted, and encodes a lysosomal alpha-glucosidase protein comprising SEQ ID NO: 173. In some such compositions, the lysosomal alpha-glucosidase coding sequence comprises the sequence set forth in any one of SEQ ID NOS: 174-182 and 205-212, optionally wherein the lysosomal alpha-glucosidase coding sequence comprises the sequence set forth in SEQ ID NO: 176. In some such compositions, the lysosomal alpha-glucosidase coding sequence consists of the sequence set forth in any one of SEQ ID NOS: 174-182 and 205-212, optionally wherein the lysosomal alpha-glucosidase coding sequence consists of the sequence set forth in SEQ ID NO: 176.

In another aspect, provided are compositions comprising a nucleic acid construct comprising a coding sequence for a multidomain therapeutic protein comprising a TfR-binding delivery domain fused to a lysosomal alpha-glucosidase, wherein the lysosomal alpha-glucosidase coding sequence is CpG-depleted relative to a wild type lysosomal alpha-glucosidase coding sequence. In some such compositions, the TfR-binding delivery domain is fused to the lysosomal alpha-glucosidase protein via a peptide linker. In some such compositions, the lysosomal alpha-glucosidase lacks the lysosomal alpha-glucosidase signal peptide and propeptide. In some such compositions, the lysosomal alpha-glucosidase comprises the sequence set forth in SEQ ID NO: 173. In some such compositions, the lysosomal alpha-glucosidase consists of the sequence set forth in SEQ ID NO: 173. In some such compositions, the lysosomal alpha-glucosidase coding sequence is codon-optimized and CpG-depleted. In some such compositions, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176. In some such compositions, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and encodes a lysosomal alpha-glucosidase protein comprising SEQ ID NO: 173, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176 and encodes a lysosomal alpha-glucosidase protein comprising SEQ ID NO: 173. In some such compositions, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182, is codon-optimized and CpG-depleted, and encodes a lysosomal alpha-glucosidase protein comprising SEQ ID NO: 173, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176, is codon-optimized and CpG-depleted, and encodes a lysosomal alpha-glucosidase protein comprising SEQ ID NO: 173. In some such compositions, the lysosomal alpha-glucosidase coding sequence comprises the sequence set forth in any one of SEQ ID NOS: 174-182, optionally wherein the lysosomal alpha-glucosidase coding sequence comprises the sequence set forth in SEQ ID NO: 176. In some such compositions, the lysosomal alpha-glucosidase coding sequence consists of the sequence set forth in any one of SEQ ID NOS: 174-182, optionally wherein the lysosomal alpha-glucosidase coding sequence consists of the sequence set forth in SEQ ID NO: 176.

In some such compositions, the coding sequence for the TfR-binding delivery domain is codon-optimized or CpG-depleted. In some such compositions, the coding sequence for the TfR-binding delivery domain is codon-optimized and CpG-depleted. In some such compositions, the TfR-binding delivery domain comprises an anti-TfR antigen-binding protein. In some such compositions, the anti-TfR antigen binding protein comprises: (i) a HCVR that comprises the HCDR1, HCDR2 and HCDR3 of a HCVR comprising the amino acid sequence set forth in SEQ ID NO: 217, 227, 237, 247, 257, 267, 277, 287, 297, 307, 317, 327, 337, 347, 357, 367, 377, 387, 397, 407, 417, 427, 437, 447, 457, 467, 477, 487, 497, 507, 517, or 527 (or a variant thereof); and/or (ii) a LCVR that comprises the LCDR1, LCDR2 and LCDR3 of a LCVR comprising the amino acid sequence set forth in SEQ ID NO: 222, 232, 242, 252, 262, 272, 282, 292, 302, 312, 322, 332, 342, 352, 362, 372, 382, 392, 402, 412, 422, 432, 442, 452, 462, 472, 482, 492, 502, 512, 522, or 532 (or a variant thereof). In some such compositions, the anti-TfR antigen binding protein comprises: (1) a HCVR comprising the HCDR1, HCDR2 and HCDR3 of a HCVR that comprises the amino acid sequence set forth in SEQ ID NO: 217 (or a variant thereof); and a LCVR comprising the LCDR1, LCDR2 and LCDR3 of a LCVR that comprises the amino acid sequence set forth in SEQ ID NO: 222 (or a variant thereof); (2) a HCVR comprising the HCDR1, HCDR2 and HCDR3 of a HCVR that comprises the amino acid sequence set forth in SEQ ID NO: 227 (or a variant thereof); and a LCVR comprising the LCDR1, LCDR2 and LCDR3 of a LCVR that comprises the amino acid sequence set forth in SEQ ID NO: 232 (or a variant thereof); (3) a HCVR comprising the HCDR1, HCDR2 and HCDR3 of a HCVR that comprises the amino acid sequence set forth in SEQ ID NO: 237 (or a variant thereof); and a LCVR comprising the LCDR1, LCDR2 and LCDR3 of a LCVR that comprises the amino acid sequence set forth in SEQ ID NO: 242 (or a variant thereof); (4) a HCVR comprising the HCDR1, HCDR2 and HCDR3 of a HCVR that comprises the amino acid sequence set forth in SEQ ID NO: 247 (or a variant thereof); and a LCVR comprising the LCDR1, LCDR2 and LCDR3 of a LCVR that comprises the amino acid sequence set forth in SEQ ID NO: 252 (or a variant thereof); (5) a HCVR comprising the HCDR1, HCDR2 and HCDR3 of a HCVR that comprises the amino acid sequence set forth in SEQ ID NO: 257 (or a variant thereof); and a LCVR comprising the LCDR1, LCDR2 and LCDR3 of a LCVR that comprises the amino acid sequence set forth in SEQ ID NO: 262 (or a variant thereof); (6) a HCVR comprising the HCDR1, HCDR2 and HCDR3 of a HCVR that comprises the amino acid sequence set forth in SEQ ID NO: 267 (or a variant thereof); and a LCVR comprising the LCDR1, LCDR2 and LCDR3 of a LCVR that comprises the amino acid sequence set forth in SEQ ID NO: 272 (or a variant thereof); (7) a HCVR comprising the HCDR1, HCDR2 and HCDR3 of a HCVR that comprises the amino acid sequence set forth in SEQ ID NO: 277 (or a variant thereof); and a LCVR comprising the LCDR1, LCDR2 and LCDR3 of a LCVR that comprises the amino acid sequence set forth in SEQ ID NO: 282 (or a variant thereof); (8) a HCVR comprising the HCDR1, HCDR2 and HCDR3 of a HCVR that comprises the amino acid sequence set forth in SEQ ID NO: 287 (or a variant thereof); and a LCVR comprising the LCDR1, LCDR2 and LCDR3 of a LCVR that comprises the amino acid sequence set forth in SEQ ID NO: 292 (or a variant thereof); (9) a HCVR comprising the HCDR1, HCDR2 and HCDR3 of a HCVR that comprises the amino acid sequence set forth in SEQ ID NO: 297 (or a variant thereof); and a LCVR comprising the LCDR1, LCDR2 and LCDR3 of a LCVR that comprises the amino acid sequence set forth in SEQ ID NO: 302 (or a variant thereof); (10) a HCVR comprising the HCDR1, HCDR2 and HCDR3 of a HCVR that comprises the amino acid sequence set forth in SEQ ID NO: 307 (or a variant thereof); and a LCVR comprising the LCDR1, LCDR2 and LCDR3 of a LCVR that comprises the amino acid sequence set forth in SEQ ID NO: 312 (or a variant thereof); (11) a HCVR comprising the HCDR1, HCDR2 and HCDR3 of a HCVR that comprises the amino acid sequence set forth in SEQ ID NO: 317 (or a variant thereof); and a LCVR comprising the LCDR1, LCDR2 and LCDR3 of a LCVR that comprises the amino acid sequence set forth in SEQ ID NO: 322 (or a variant thereof); (12) a HCVR comprising the HCDR1, HCDR2 and HCDR3 of a HCVR that comprises the amino acid sequence set forth in SEQ ID NO: 327 (or a variant thereof); and a LCVR comprising the LCDR1, LCDR2 and LCDR3 of a LCVR that comprises the amino acid sequence set forth in SEQ ID NO: 332 (or a variant thereof); (13) a HCVR comprising the HCDR1, HCDR2 and HCDR3 of a HCVR that comprises the amino acid sequence set forth in SEQ ID NO: 337 (or a variant thereof); and a LCVR comprising the LCDR1, LCDR2 and LCDR3 of a LCVR that comprises the amino acid sequence set forth in SEQ ID NO: 342 (or a variant thereof); (14) a HCVR comprising the HCDR1, HCDR2 and HCDR3 of a HCVR that comprises the amino acid sequence set forth in SEQ ID NO: 347 (or a variant thereof); and a LCVR comprising the LCDR1, LCDR2 and LCDR3 of a LCVR that comprises the amino acid sequence set forth in SEQ ID NO: 352 (or a variant thereof); (15) a HCVR comprising the HCDR1, HCDR2 and HCDR3 of a HCVR that comprises the amino acid sequence set forth in SEQ ID NO: 357 (or a variant thereof); and a LCVR comprising the LCDR1, LCDR2 and LCDR3 of a LCVR that comprises the amino acid sequence set forth in SEQ ID NO: 362 (or a variant thereof); (16) a HCVR comprising the HCDR1, HCDR2 and HCDR3 of a HCVR that comprises the amino acid sequence set forth in SEQ ID NO: 367 (or a variant thereof); and a LCVR comprising the LCDR1, LCDR2 and LCDR3 of a LCVR that comprises the amino acid sequence set forth in SEQ ID NO: 372 (or a variant thereof); (17) a HCVR comprising the HCDR1, HCDR2 and HCDR3 of a HCVR that comprises the amino acid sequence set forth in SEQ ID NO: 377 (or a variant thereof); and a LCVR comprising the LCDR1, LCDR2 and LCDR3 of a LCVR that comprises the amino acid sequence set forth in SEQ ID NO: 382 (or a variant thereof); (18) a HCVR comprising the HCDR1, HCDR2 and HCDR3 of a HCVR that comprises the amino acid sequence set forth in SEQ ID NO: 387 (or a variant thereof); and a LCVR comprising the LCDR1, LCDR2 and LCDR3 of a LCVR that comprises the amino acid sequence set forth in SEQ ID NO: 392 (or a variant thereof); (19) a HCVR comprising the HCDR1, HCDR2 and HCDR3 of a HCVR that comprises the amino acid sequence set forth in SEQ ID NO: 397 (or a variant thereof); and a LCVR comprising the LCDR1, LCDR2 and LCDR3 of a LCVR that comprises the amino acid sequence set forth in SEQ ID NO: 402 (or a variant thereof); (20) a HCVR comprising the HCDR1, HCDR2 and HCDR3 of a HCVR that comprises the amino acid sequence set forth in SEQ ID NO: 407 (or a variant thereof); and a LCVR comprising the LCDR1, LCDR2 and LCDR3 of a LCVR that comprises the amino acid sequence set forth in SEQ ID NO: 412 (or a variant thereof); (21) a HCVR comprising the HCDR1, HCDR2 and HCDR3 of a HCVR that comprises the amino acid sequence set forth in SEQ ID NO: 417 (or a variant thereof); and a LCVR comprising the LCDR1, LCDR2 and LCDR3 of a LCVR that comprises the amino acid sequence set forth in SEQ ID NO: 422 (or a variant thereof); (22) a HCVR comprising the HCDR1, HCDR2 and HCDR3 of a HCVR that comprises the amino acid sequence set forth in SEQ ID NO: 427 (or a variant thereof); and a LCVR comprising the LCDR1, LCDR2 and LCDR3 of a LCVR that comprises the amino acid sequence set forth in SEQ ID NO: 432 (or a variant thereof); (23) a HCVR comprising the HCDR1, HCDR2 and HCDR3 of a HCVR that comprises the amino acid sequence set forth in SEQ ID NO: 437 (or a variant thereof); and a LCVR comprising the LCDR1, LCDR2 and LCDR3 of a LCVR that comprises the amino acid sequence set forth in SEQ ID NO: 442 (or a variant thereof); (24) a HCVR comprising the HCDR1, HCDR2 and HCDR3 of a HCVR that comprises the amino acid sequence set forth in SEQ ID NO: 447 (or a variant thereof); and a LCVR comprising the LCDR1, LCDR2 and LCDR3 of a LCVR that comprises the amino acid sequence set forth in SEQ ID NO: 452 (or a variant thereof); (25) a HCVR comprising the HCDR1, HCDR2 and HCDR3 of a HCVR that comprises the amino acid sequence set forth in SEQ ID NO: 457 (or a variant thereof); and a LCVR comprising the LCDR1, LCDR2 and LCDR3 of a LCVR that comprises the amino acid sequence set forth in SEQ ID NO: 462 (or a variant thereof); (26) a HCVR comprising the HCDR1, HCDR2 and HCDR3 of a HCVR that comprises the amino acid sequence set forth in SEQ ID NO: 467 (or a variant thereof); and a LCVR comprising the LCDR1, LCDR2 and LCDR3 of a LCVR that comprises the amino acid sequence set forth in SEQ ID NO: 472 (or a variant thereof); (27) a HCVR comprising the HCDR1, HCDR2 and HCDR3 of a HCVR that comprises the amino acid sequence set forth in SEQ ID NO: 477 (or a variant thereof); and a LCVR comprising the LCDR1, LCDR2 and LCDR3 of a LCVR that comprises the amino acid sequence set forth in SEQ ID NO: 482 (or a variant thereof); (28) a HCVR comprising the HCDR1, HCDR2 and HCDR3 of a HCVR that comprises the amino acid sequence set forth in SEQ ID NO: 487 (or a variant thereof); and a LCVR comprising the LCDR1, LCDR2 and LCDR3 of a LCVR that comprises the amino acid sequence set forth in SEQ ID NO: 492 (or a variant thereof); (29) a HCVR comprising the HCDR1, HCDR2 and HCDR3 of a HCVR that comprises the amino acid sequence set forth in SEQ ID NO: 497 (or a variant thereof); and a LCVR comprising the LCDR1, LCDR2 and LCDR3 of a LCVR that comprises the amino acid sequence set forth in SEQ ID NO: 502 (or a variant thereof); (30) a HCVR comprising the HCDR1, HCDR2 and HCDR3 of a HCVR that comprises the amino acid sequence set forth in SEQ ID NO: 507 (or a variant thereof); and a LCVR comprising the LCDR1, LCDR2 and LCDR3 of a LCVR that comprises the amino acid sequence set forth in SEQ ID NO: 512 (or a variant thereof); (31) a HCVR comprising the HCDR1, HCDR2 and HCDR3 of a HCVR that comprises the amino acid sequence set forth in SEQ ID NO: 517 (or a variant thereof); and a LCVR comprising the LCDR1, LCDR2 and LCDR3 of a LCVR that comprises the amino acid sequence set forth in SEQ ID NO: 522 (or a variant thereof); or (32) a HCVR comprising the HCDR1, HCDR2 and HCDR3 of a HCVR that comprises the amino acid sequence set forth in SEQ ID NO: 527 (or a variant thereof); and a LCVR comprising the LCDR1, LCDR2 and LCDR3 of a LCVR that comprises the amino acid sequence set forth in SEQ ID NO: 532 (or a variant thereof). In some such compositions, the anti-TfR antigen binding protein comprises: (1) a HCVR comprising the HCDR1, HCDR2 and HCDR3 of a HCVR that comprises the amino acid sequence set forth in SEQ ID NO: 437 (or a variant thereof); and a LCVR comprising the LCDR1, LCDR2 and LCDR3 of a LCVR that comprises the amino acid sequence set forth in SEQ ID NO: 442 (or a variant thereof); or (2) a HCVR comprising the HCDR1, HCDR2 and HCDR3 of a HCVR that comprises the amino acid sequence set forth in SEQ ID NO: 457 (or a variant thereof); and a LCVR comprising the LCDR1, LCDR2 and LCDR3 of a LCVR that comprises the amino acid sequence set forth in SEQ ID NO: 462 (or a variant thereof). In some such compositions, the anti-TfR antigen binding protein comprises: (a) a HCVR that comprises: an HCDR1 comprising the amino acid sequence set forth in SEQ ID NO: 218 (or a variant thereof), an HCDR2 comprising the amino acid sequence set forth in SEQ ID NO: 219 (or a variant thereof), and an HCDR3 comprising the amino acid sequence set forth in SEQ ID NO: 220 (or a variant thereof); and a LCVR that comprises: an LCDR1 comprising the amino acid sequence set forth in SEQ ID NO: 223 (or a variant thereof), an LCDR2 comprising the amino acid sequence set forth in SEQ ID NO: 224 (or a variant thereof), and an LCDR3 comprising the amino acid sequence set forth in SEQ ID NO: 225 (or a variant thereof); (b) a HCVR that comprises: an HCDR1 comprising the amino acid sequence set forth in SEQ ID NO: 228 (or a variant thereof), an HCDR2 comprising the amino acid sequence set forth in SEQ ID NO: 229 (or a variant thereof), and an HCDR3 comprising the amino acid sequence set forth in SEQ ID NO: 230 (or a variant thereof); and a LCVR that comprises: an LCDR1 comprising the amino acid sequence set forth in SEQ ID NO: 233 (or a variant thereof), an LCDR2 comprising the amino acid sequence set forth in SEQ ID NO: 234 (or a variant thereof), and an LCDR3 comprising the amino acid sequence set forth in SEQ ID NO: 235 (or a variant thereof); (c) a HCVR that comprises: an HCDR1 comprising the amino acid sequence set forth in SEQ ID NO: 238 (or a variant thereof), an HCDR2 comprising the amino acid sequence set forth in SEQ ID NO: 239 (or a variant thereof), and an HCDR3 comprising the amino acid sequence set forth in SEQ ID NO: 240 (or a variant thereof); and a LCVR that comprises: an LCDR1 comprising the amino acid sequence set forth in SEQ ID NO: 243 (or a variant thereof), an LCDR2 comprising the amino acid sequence set forth in SEQ ID NO: 244 (or a variant thereof), and an LCDR3 comprising the amino acid sequence set forth in SEQ ID NO: 245 (or a variant thereof); (d) a HCVR that comprises: an HCDR1 comprising the amino acid sequence set forth in SEQ ID NO: 248 (or a variant thereof), an HCDR2 comprising the amino acid sequence set forth in SEQ ID NO: 249 (or a variant thereof), and an HCDR3 comprising the amino acid sequence set forth in SEQ ID NO: 250 (or a variant thereof); and a LCVR that comprises: an LCDR1 comprising the amino acid sequence set forth in SEQ ID NO: 253 (or a variant thereof), an LCDR2 comprising the amino acid sequence set forth in SEQ ID NO: 254 (or a variant thereof), and an LCDR3 comprising the amino acid sequence set forth in SEQ ID NO: 255 (or a variant thereof); (e) a HCVR that comprises: an HCDR1 comprising the amino acid sequence set forth in SEQ ID NO: 258 (or a variant thereof), an HCDR2 comprising the amino acid sequence set forth in SEQ ID NO: 259 (or a variant thereof), and an HCDR3 comprising the amino acid sequence set forth in SEQ ID NO: 260 (or a variant thereof); and a LCVR that comprises: an LCDR1 comprising the amino acid sequence set forth in SEQ ID NO: 263 (or a variant thereof), an LCDR2 comprising the amino acid sequence set forth in SEQ ID NO: 264 (or a variant thereof), and an LCDR3 comprising the amino acid sequence set forth in SEQ ID NO: 265 (or a variant thereof); (f) a HCVR that comprises: an HCDR1 comprising the amino acid sequence set forth in SEQ ID NO: 268 (or a variant thereof), an HCDR2 comprising the amino acid sequence set forth in SEQ ID NO: 269 (or a variant thereof), and an HCDR3 comprising the amino acid sequence set forth in SEQ ID NO: 270 (or a variant thereof); and a LCVR that comprises: an LCDR1 comprising the amino acid sequence set forth in SEQ ID NO: 273 (or a variant thereof), an LCDR2 comprising the amino acid sequence set forth in SEQ ID NO: 274 (or a variant thereof), and an LCDR3 comprising the amino acid sequence set forth in SEQ ID NO: 275 (or a variant thereof); (g) a HCVR that comprises: an HCDR1 comprising the amino acid sequence set forth in SEQ ID NO: 278 (or a variant thereof), an HCDR2 comprising the amino acid sequence set forth in SEQ ID NO: 279 (or a variant thereof), and an HCDR3 comprising the amino acid sequence set forth in SEQ ID NO: 280 (or a variant thereof); and a LCVR that comprises: an LCDR1 comprising the amino acid sequence set forth in SEQ ID NO: 283 (or a variant thereof), an LCDR2 comprising the amino acid sequence set forth in SEQ ID NO: 284 (or a variant thereof), and an LCDR3 comprising the amino acid sequence set forth in SEQ ID NO: 285 (or a variant thereof); (h) a HCVR that comprises: an HCDR1 comprising the amino acid sequence set forth in SEQ ID NO: 288 (or a variant thereof), an HCDR2 comprising the amino acid sequence set forth in SEQ ID NO: 289 (or a variant thereof), and an HCDR3 comprising the amino acid sequence set forth in SEQ ID NO: 290 (or a variant thereof); and a LCVR that comprises: an LCDR1 comprising the amino acid sequence set forth in SEQ ID NO: 293 (or a variant thereof), an LCDR2 comprising the amino acid sequence set forth in SEQ ID NO: 294 (or a variant thereof), and an LCDR3 comprising the amino acid sequence set forth in SEQ ID NO: 295 (or a variant thereof); (i) a HCVR that comprises: an HCDR1 comprising the amino acid sequence set forth in SEQ ID NO: 298 (or a variant thereof), an HCDR2 comprising the amino acid sequence set forth in SEQ ID NO: 299 (or a variant thereof), and an HCDR3 comprising the amino acid sequence set forth in SEQ ID NO: 300 (or a variant thereof); and a LCVR that comprises: an LCDR1 comprising the amino acid sequence set forth in SEQ ID NO: 303 (or a variant thereof), an LCDR2 comprising the amino acid sequence set forth in SEQ ID NO: 304 (or a variant thereof), and an LCDR3 comprising the amino acid sequence set forth in SEQ ID NO: 305 (or a variant thereof); (j) a HCVR that comprises: an HCDR1 comprising the amino acid sequence set forth in SEQ ID NO: 308 (or a variant thereof), an HCDR2 comprising the amino acid sequence set forth in SEQ ID NO: 309 (or a variant thereof), and an HCDR3 comprising the amino acid sequence set forth in SEQ ID NO: 310 (or a variant thereof); and a LCVR that comprises: an LCDR1 comprising the amino acid sequence set forth in SEQ ID NO: 313 (or a variant thereof), an LCDR2 comprising the amino acid sequence set forth in SEQ ID NO: 314 (or a variant thereof), and an LCDR3 comprising the amino acid sequence set forth in SEQ ID NO: 315 (or a variant thereof); (k) a HCVR that comprises: an HCDR1 comprising the amino acid sequence set forth in SEQ ID NO: 318 (or a variant thereof), an HCDR2 comprising the amino acid sequence set forth in SEQ ID NO: 319 (or a variant thereof), and an HCDR3 comprising the amino acid sequence set forth in SEQ ID NO: 320 (or a variant thereof); and a LCVR that comprises: an LCDR1 comprising the amino acid sequence set forth in SEQ ID NO: 323 (or a variant thereof), an LCDR2 comprising the amino acid sequence set forth in SEQ ID NO: 324 (or a variant thereof), and an LCDR3 comprising the amino acid sequence set forth in SEQ ID NO: 325 (or a variant thereof); (1) a HCVR that comprises: an HCDR1 comprising the amino acid sequence set forth in SEQ ID NO: 328 (or a variant thereof), an HCDR2 comprising the amino acid sequence set forth in SEQ ID NO: 329 (or a variant thereof), and an HCDR3 comprising the amino acid sequence set forth in SEQ ID NO: 330 (or a variant thereof); and a LCVR that comprises: an LCDR1 comprising the amino acid sequence set forth in SEQ ID NO: 333 (or a variant thereof), an LCDR2 comprising the amino acid sequence set forth in SEQ ID NO: 334 (or a variant thereof), and an LCDR3 comprising the amino acid sequence set forth in SEQ ID NO: 335 (or a variant thereof); (m) a HCVR that comprises: an HCDR1 comprising the amino acid sequence set forth in SEQ ID NO: 338 (or a variant thereof), an HCDR2 comprising the amino acid sequence set forth in SEQ ID NO: 339 (or a variant thereof), and an HCDR3 comprising the amino acid sequence set forth in SEQ ID NO: 340 (or a variant thereof); and a LCVR that comprises: an LCDR1 comprising the amino acid sequence set forth in SEQ ID NO: 343 (or a variant thereof), an LCDR2 comprising the amino acid sequence set forth in SEQ ID NO: 344 (or a variant thereof), and an LCDR3 comprising the amino acid sequence set forth in SEQ ID NO: 345 (or a variant thereof); (n) a HCVR that comprises: an HCDR1 comprising the amino acid sequence set forth in SEQ ID NO: 348 (or a variant thereof), an HCDR2 comprising the amino acid sequence set forth in SEQ ID NO: 349 (or a variant thereof), and an HCDR3 comprising the amino acid sequence set forth in SEQ ID NO: 350 (or a variant thereof); and a LCVR that comprises: an LCDR1 comprising the amino acid sequence set forth in SEQ ID NO: 353 (or a variant thereof), an LCDR2 comprising the amino acid sequence set forth in SEQ ID NO: 354 (or a variant thereof), and an LCDR3 comprising the amino acid sequence set forth in SEQ ID NO: 355 (or a variant thereof); (o) a HCVR that comprises: an HCDR1 comprising the amino acid sequence set forth in SEQ ID NO: 358 (or a variant thereof), an HCDR2 comprising the amino acid sequence set forth in SEQ ID NO: 359 (or a variant thereof), and an HCDR3 comprising the amino acid sequence set forth in SEQ ID NO: 360 (or a variant thereof); and a LCVR that comprises: an LCDR1 comprising the amino acid sequence set forth in SEQ ID NO: 363 (or a variant thereof), an LCDR2 comprising the amino acid sequence set forth in SEQ ID NO: 364 (or a variant thereof), and an LCDR3 comprising the amino acid sequence set forth in SEQ ID NO: 365 (or a variant thereof); (p) a HCVR that comprises: an HCDR1 comprising the amino acid sequence set forth in SEQ ID NO: 368 (or a variant thereof), an HCDR2 comprising the amino acid sequence set forth in SEQ ID NO: 369 (or a variant thereof), and an HCDR3 comprising the amino acid sequence set forth in SEQ ID NO: 370 (or a variant thereof); and a LCVR that comprises: an LCDR1 comprising the amino acid sequence set forth in SEQ ID NO: 373 (or a variant thereof), an LCDR2 comprising the amino acid sequence set forth in SEQ ID NO: 374 (or a variant thereof), and an LCDR3 comprising the amino acid sequence set forth in SEQ ID NO: 375 (or a variant thereof); (q) a HCVR that comprises: an HCDR1 comprising the amino acid sequence set forth in SEQ ID NO: 378 (or a variant thereof), an HCDR2 comprising the amino acid sequence set forth in SEQ ID NO: 379 (or a variant thereof), and an HCDR3 comprising the amino acid sequence set forth in SEQ ID NO: 380 (or a variant thereof); and a LCVR that comprises: an LCDR1 comprising the amino acid sequence set forth in SEQ ID NO: 383 (or a variant thereof), an LCDR2 comprising the amino acid sequence set forth in SEQ ID NO: 384 (or a variant thereof), and an LCDR3 comprising the amino acid sequence set forth in SEQ ID NO: 385 (or a variant thereof); (r) a HCVR that comprises: an HCDR1 comprising the amino acid sequence set forth in SEQ ID NO: 388 (or a variant thereof), an HCDR2 comprising the amino acid sequence set forth in SEQ ID NO: 389 (or a variant thereof), and an HCDR3 comprising the amino acid sequence set forth in SEQ ID NO: 390 (or a variant thereof); and a LCVR that comprises: an LCDR1 comprising the amino acid sequence set forth in SEQ ID NO: 393 (or a variant thereof), an LCDR2 comprising the amino acid sequence set forth in SEQ ID NO: 394 (or a variant thereof), and an LCDR3 comprising the amino acid sequence set forth in SEQ ID NO: 395 (or a variant thereof); (s) a HCVR that comprises: an HCDR1 comprising the amino acid sequence set forth in SEQ ID NO: 398 (or a variant thereof), an HCDR2 comprising the amino acid sequence set forth in SEQ ID NO: 399 (or a variant thereof), and an HCDR3 comprising the amino acid sequence set forth in SEQ ID NO: 400 (or a variant thereof); and a LCVR that comprises: an LCDR1 comprising the amino acid sequence set forth in SEQ ID NO: 403 (or a variant thereof), an LCDR2 comprising the amino acid sequence set forth in SEQ ID NO: 404 (or a variant thereof), and an LCDR3 comprising the amino acid sequence set forth in SEQ ID NO: 405 (or a variant thereof); (t) a HCVR that comprises: an HCDR1 comprising the amino acid sequence set forth in SEQ ID NO: 408 (or a variant thereof), an HCDR2 comprising the amino acid sequence set forth in SEQ ID NO: 409 (or a variant thereof), and an HCDR3 comprising the amino acid sequence set forth in SEQ ID NO: 410 (or a variant thereof); and a LCVR that comprises: an LCDR1 comprising the amino acid sequence set forth in SEQ ID NO: 413 (or a variant thereof), an LCDR2 comprising the amino acid sequence set forth in SEQ ID NO: 414 (or a variant thereof), and an LCDR3 comprising the amino acid sequence set forth in SEQ ID NO: 415 (or a variant thereof); (u) a HCVR that comprises: an HCDR1 comprising the amino acid sequence set forth in SEQ ID NO: 418 (or a variant thereof), an HCDR2 comprising the amino acid sequence set forth in SEQ ID NO: 419 (or a variant thereof), and an HCDR3 comprising the amino acid sequence set forth in SEQ ID NO: 420 (or a variant thereof); and a LCVR that comprises: an LCDR1 comprising the amino acid sequence set forth in SEQ ID NO: 423 (or a variant thereof), an LCDR2 comprising the amino acid sequence set forth in SEQ ID NO: 424 (or a variant thereof), and an LCDR3 comprising the amino acid sequence set forth in SEQ ID NO: 425 (or a variant thereof); (v) a HCVR that comprises: an HCDR1 comprising the amino acid sequence set forth in SEQ ID NO: 428 (or a variant thereof), an HCDR2 comprising the amino acid sequence set forth in SEQ ID NO: 429 (or a variant thereof), and an HCDR3 comprising the amino acid sequence set forth in SEQ ID NO: 430 (or a variant thereof); and a LCVR that comprises: an LCDR1 comprising the amino acid sequence set forth in SEQ ID NO: 433 (or a variant thereof), an LCDR2 comprising the amino acid sequence set forth in SEQ ID NO: 434 (or a variant thereof), and an LCDR3 comprising the amino acid sequence set forth in SEQ ID NO: 435 (or a variant thereof); (w) a HCVR that comprises: an HCDR1 comprising the amino acid sequence set forth in SEQ ID NO: 438 (or a variant thereof), an HCDR2 comprising the amino acid sequence set forth in SEQ ID NO: 439 (or a variant thereof), and an HCDR3 comprising the amino acid sequence set forth in SEQ ID NO: 440 (or a variant thereof); and a LCVR that comprises: an LCDR1 comprising the amino acid sequence set forth in SEQ ID NO: 443 (or a variant thereof), an LCDR2 comprising the amino acid sequence set forth in SEQ ID NO: 444 (or a variant thereof), and an LCDR3 comprising the amino acid sequence set forth in SEQ ID NO: 445 (or a variant thereof); (x) a HCVR that comprises: an HCDR1 comprising the amino acid sequence set forth in SEQ ID NO: 448 (or a variant thereof), an HCDR2 comprising the amino acid sequence set forth in SEQ ID NO: 449 (or a variant thereof), and an HCDR3 comprising the amino acid sequence set forth in SEQ ID NO: 450 (or a variant thereof); and a LCVR that comprises: an LCDR1 comprising the amino acid sequence set forth in SEQ ID NO: 453 (or a variant thereof), an LCDR2 comprising the amino acid sequence set forth in SEQ ID NO: 454 (or a variant thereof), and an LCDR3 comprising the amino acid sequence set forth in SEQ ID NO: 455 (or a variant thereof); (y) a HCVR that comprises: an HCDR1 comprising the amino acid sequence set forth in SEQ ID NO: 458 (or a variant thereof), an HCDR2 comprising the amino acid sequence set forth in SEQ ID NO: 459 (or a variant thereof), and an HCDR3 comprising the amino acid sequence set forth in SEQ ID NO: 460 (or a variant thereof); and a LCVR that comprises: an LCDR1 comprising the amino acid sequence set forth in SEQ ID NO: 463 (or a variant thereof), an LCDR2 comprising the amino acid sequence set forth in SEQ ID NO: 464 (or a variant thereof), and an LCDR3 comprising the amino acid sequence set forth in SEQ ID NO: 465 (or a variant thereof); (z) a HCVR that comprises: an HCDR1 comprising the amino acid sequence set forth in SEQ ID NO: 468 (or a variant thereof), an HCDR2 comprising the amino acid sequence set forth in SEQ ID NO: 469 (or a variant thereof), and an HCDR3 comprising the amino acid sequence set forth in SEQ ID NO: 470 (or a variant thereof); and a LCVR that comprises: an LCDR1 comprising the amino acid sequence set forth in SEQ ID NO: 473 (or a variant thereof), an LCDR2 comprising the amino acid sequence set forth in SEQ ID NO: 474 (or a variant thereof), and an LCDR3 comprising the amino acid sequence set forth in SEQ ID NO: 475 (or a variant thereof); (aa) a HCVR that comprises: an HCDR1 comprising the amino acid sequence set forth in SEQ ID NO: 478 (or a variant thereof), an HCDR2 comprising the amino acid sequence set forth in SEQ ID NO: 479 (or a variant thereof), and an HCDR3 comprising the amino acid sequence set forth in SEQ ID NO: 480 (or a variant thereof); and a LCVR that comprises: an LCDR1 comprising the amino acid sequence set forth in SEQ ID NO: 483 (or a variant thereof), an LCDR2 comprising the amino acid sequence set forth in SEQ ID NO: 484 (or a variant thereof), and an LCDR3 comprising the amino acid sequence set forth in SEQ ID NO: 485 (or a variant thereof); (ab) a HCVR that comprises: an HCDR1 comprising the amino acid sequence set forth in SEQ ID NO: 488 (or a variant thereof), an HCDR2 comprising the amino acid sequence set forth in SEQ ID NO: 489 (or a variant thereof), and an HCDR3 comprising the amino acid sequence set forth in SEQ ID NO: 490 (or a variant thereof); and a LCVR that comprises: an LCDR1 comprising the amino acid sequence set forth in SEQ ID NO: 493 (or a variant thereof), an LCDR2 comprising the amino acid sequence set forth in SEQ ID NO: 494 (or a variant thereof), and an LCDR3 comprising the amino acid sequence set forth in SEQ ID NO: 495 (or a variant thereof); (ac) a HCVR that comprises: an HCDR1 comprising the amino acid sequence set forth in SEQ ID NO: 498 (or a variant thereof), an HCDR2 comprising the amino acid sequence set forth in SEQ ID NO: 499 (or a variant thereof), and an HCDR3 comprising the amino acid sequence set forth in SEQ ID NO: 500 (or a variant thereof); and a LCVR that comprises: an LCDR1 comprising the amino acid sequence set forth in SEQ ID NO: 503 (or a variant thereof), an LCDR2 comprising the amino acid sequence set forth in SEQ ID NO: 504 (or a variant thereof), and an LCDR3 comprising the amino acid sequence set forth in SEQ ID NO: 505 (or a variant thereof); (ad) a HCVR that comprises: an HCDR1 comprising the amino acid sequence set forth in SEQ ID NO: 508 (or a variant thereof), an HCDR2 comprising the amino acid sequence set forth in SEQ ID NO: 509 (or a variant thereof), and an HCDR3 comprising the amino acid sequence set forth in SEQ ID NO: 510 (or a variant thereof); and a LCVR that comprises: an LCDR1 comprising the amino acid sequence set forth in SEQ ID NO: 513 (or a variant thereof), an LCDR2 comprising the amino acid sequence set forth in SEQ ID NO: 514 (or a variant thereof), and an LCDR3 comprising the amino acid sequence set forth in SEQ ID NO: 515 (or a variant thereof); (ae) a HCVR that comprises: an HCDR1 comprising the amino acid sequence set forth in SEQ ID NO: 518 (or a variant thereof), an HCDR2 comprising the amino acid sequence set forth in SEQ ID NO: 519 (or a variant thereof), and an HCDR3 comprising the amino acid sequence set forth in SEQ ID NO: 520 (or a variant thereof); and a LCVR that comprises: an LCDR1 comprising the amino acid sequence set forth in SEQ ID NO: 523 (or a variant thereof), an LCDR2 comprising the amino acid sequence set forth in SEQ ID NO: 524 (or a variant thereof), and an LCDR3 comprising the amino acid sequence set forth in SEQ ID NO: 525 (or a variant thereof); and/or (af) a HCVR that comprises: an HCDR1 comprising the amino acid sequence set forth in SEQ ID NO: 528 (or a variant thereof), an HCDR2 comprising the amino acid sequence set forth in SEQ ID NO: 529 (or a variant thereof), and an HCDR3 comprising the amino acid sequence set forth in SEQ ID NO: 530 (or a variant thereof); and a LCVR that comprises: an LCDR1 comprising the amino acid sequence set forth in SEQ ID NO: 533 (or a variant thereof), an LCDR2 comprising the amino acid sequence set forth in SEQ ID NO: 534 (or a variant thereof), and an LCDR3 comprising the amino acid sequence set forth in SEQ ID NO: 535 (or a variant thereof). In some such compositions, the anti-TfR antigen binding protein comprises: (a) a HCVR that comprises: an HCDR1 comprising the amino acid sequence set forth in SEQ ID NO: 438 (or a variant thereof), an HCDR2 comprising the amino acid sequence set forth in SEQ ID NO: 439 (or a variant thereof), and an HCDR3 comprising the amino acid sequence set forth in SEQ ID NO: 440 (or a variant thereof); and a LCVR that comprises: an LCDR1 comprising the amino acid sequence set forth in SEQ ID NO: 443 (or a variant thereof), an LCDR2 comprising the amino acid sequence set forth in SEQ ID NO: 444 (or a variant thereof), and an LCDR3 comprising the amino acid sequence set forth in SEQ ID NO: 445 (or a variant thereof); or (b) a HCVR that comprises: an HCDR1 comprising the amino acid sequence set forth in SEQ ID NO: 458 (or a variant thereof), an HCDR2 comprising the amino acid sequence set forth in SEQ ID NO: 459 (or a variant thereof), and an HCDR3 comprising the amino acid sequence set forth in SEQ ID NO: 460 (or a variant thereof); and a LCVR that comprises: an LCDR1 comprising the amino acid sequence set forth in SEQ ID NO: 463 (or a variant thereof), an LCDR2 comprising the amino acid sequence set forth in SEQ ID NO: 464 (or a variant thereof), and an LCDR3 comprising the amino acid sequence set forth in SEQ ID NO: 465 (or a variant thereof). In some such compositions, the anti-TfR antigen binding protein comprises: (i) a HCVR that comprises the amino acid sequence set forth in SEQ ID NO: 217 (or a variant thereof); and a LCVR that comprises the amino acid sequence set forth in SEQ ID NO: 222 (or a variant thereof); (ii) a HCVR that comprises the amino acid sequence set forth in SEQ ID NO: 227 (or a variant thereof); and a LCVR that comprises the amino acid sequence set forth in SEQ ID NO: 232 (or a variant thereof); (iii) a HCVR that comprises the amino acid sequence set forth in SEQ ID NO: 237 (or a variant thereof); and a LCVR that comprises the amino acid sequence set forth in SEQ ID NO: 242 (or a variant thereof); (iv) a HCVR that comprises the amino acid sequence set forth in SEQ ID NO: 247 (or a variant thereof); and a LCVR that comprises the amino acid sequence set forth in SEQ ID NO: 252 (or a variant thereof); (v) a HCVR that comprises the amino acid sequence set forth in SEQ ID NO: 257 (or a variant thereof); and a LCVR that comprises the amino acid sequence set forth in SEQ ID NO: 262 (or a variant thereof); (vi) a HCVR that comprises the amino acid sequence set forth in SEQ ID NO: 267 (or a variant thereof); and a LCVR that comprises the amino acid sequence set forth in SEQ ID NO: 272 (or a variant thereof); (vii) a HCVR that comprises the amino acid sequence set forth in SEQ ID NO: 277 (or a variant thereof); and a LCVR that comprises the amino acid sequence set forth in SEQ ID NO: 282 (or a variant thereof); (viii) a HCVR that comprises the amino acid sequence set forth in SEQ ID NO: 287 (or a variant thereof); and a LCVR that comprises the amino acid sequence set forth in SEQ ID NO: 292 (or a variant thereof); (ix) a HCVR that comprises the amino acid sequence set forth in SEQ ID NO: 297 (or a variant thereof); and a LCVR that comprises the amino acid sequence set forth in SEQ ID NO: 302 (or a variant thereof); (x) a HCVR that comprises the amino acid sequence set forth in SEQ ID NO: 307 (or a variant thereof); and a LCVR that comprises the amino acid sequence set forth in SEQ ID NO: 312 (or a variant thereof); (xi) a HCVR that comprises the amino acid sequence set forth in SEQ ID NO: 317 (or a variant thereof); and a LCVR that comprises the amino acid sequence set forth in SEQ ID NO: 322 (or a variant thereof); (xii) a HCVR that comprises the amino acid sequence set forth in SEQ ID NO: 327 (or a variant thereof); and a LCVR that comprises the amino acid sequence set forth in SEQ ID NO: 332 (or a variant thereof); (xiii) a HCVR that comprises the amino acid sequence set forth in SEQ ID NO: 337 (or a variant thereof); and a LCVR that comprises the amino acid sequence set forth in SEQ ID NO: 342 (or a variant thereof); (xiv) a HCVR that comprises the amino acid sequence set forth in SEQ ID NO: 347 (or a variant thereof); and a LCVR that comprises the amino acid sequence set forth in SEQ ID NO: 352 (or a variant thereof); (xv) a HCVR that comprises the amino acid sequence set forth in SEQ ID NO: 357 (or a variant thereof); and a LCVR that comprises the amino acid sequence set forth in SEQ ID NO: 362 (or a variant thereof); (xvi) a HCVR that comprises the amino acid sequence set forth in SEQ ID NO: 367 (or a variant thereof); and a LCVR that comprises the amino acid sequence set forth in SEQ ID NO: 372 (or a variant thereof); (xvii) a HCVR that comprises the amino acid sequence set forth in SEQ ID NO: 377 (or a variant thereof); and a LCVR that comprises the amino acid sequence set forth in SEQ ID NO: 382 (or a variant thereof); (xviii) a HCVR that comprises the amino acid sequence set forth in SEQ ID NO: 387 (or a variant thereof); and a LCVR that comprises the amino acid sequence set forth in SEQ ID NO: 392 (or a variant thereof); (xix) a HCVR that comprises the amino acid sequence set forth in SEQ ID NO: 397 (or a variant thereof); and a LCVR that comprises the amino acid sequence set forth in SEQ ID NO: 402 (or a variant thereof); (xx) a HCVR that comprises the amino acid sequence set forth in SEQ ID NO: 407 (or a variant thereof); and a LCVR that comprises the amino acid sequence set forth in SEQ ID NO: 412 (or a variant thereof); (xxi) a HCVR that comprises the amino acid sequence set forth in SEQ ID NO: 417 (or a variant thereof); and a LCVR that comprises the amino acid sequence set forth in SEQ ID NO: 422 (or a variant thereof); (xxii) a HCVR that comprises the amino acid sequence set forth in SEQ ID NO: 427 (or a variant thereof); and a LCVR that comprises the amino acid sequence set forth in SEQ ID NO: 432 (or a variant thereof); (xxiii) a HCVR that comprises the amino acid sequence set forth in SEQ ID NO: 437 (or a variant thereof); and a LCVR that comprises the amino acid sequence set forth in SEQ ID NO: 442 (or a variant thereof); (xxiv) a HCVR that comprises the amino acid sequence set forth in SEQ ID NO: 447 (or a variant thereof); and a LCVR that comprises the amino acid sequence set forth in SEQ ID NO: 452 (or a variant thereof); (xxv) a HCVR that comprises the amino acid sequence set forth in SEQ ID NO: 457 (or a variant thereof); and a LCVR that comprises the amino acid sequence set forth in SEQ ID NO: 462 (or a variant thereof); (xxvi) a HCVR that comprises the amino acid sequence set forth in SEQ ID NO: 467 (or a variant thereof); and a LCVR that comprises the amino acid sequence set forth in SEQ ID NO: 472 (or a variant thereof); (xxvii) a HCVR that comprises the amino acid sequence set forth in SEQ ID NO: 477 (or a variant thereof); and a LCVR that comprises the amino acid sequence set forth in SEQ ID NO: 482 (or a variant thereof); (xxviii) a HCVR that comprises the amino acid sequence set forth in SEQ ID NO: 487 (or a variant thereof); and a LCVR that comprises the amino acid sequence set forth in SEQ ID NO: 492 (or a variant thereof); (xxix) a HCVR that comprises the amino acid sequence set forth in SEQ ID NO: 497 (or a variant thereof); and a LCVR that comprises the amino acid sequence set forth in SEQ ID NO: 502 (or a variant thereof); (xxx) a HCVR that comprises the amino acid sequence set forth in SEQ ID NO: 507 (or a variant thereof); and a LCVR that comprises the amino acid sequence set forth in SEQ ID NO: 512 (or a variant thereof); (xxxi) a HCVR that comprises the amino acid sequence set forth in SEQ ID NO: 517 (or a variant thereof); and a LCVR that comprises the amino acid sequence set forth in SEQ ID NO: 522 (or a variant thereof); and/or (xxxii) a HCVR that comprises the amino acid sequence set forth in SEQ ID NO: 527 (or a variant thereof); and a LCVR that comprises the amino acid sequence set forth in SEQ ID NO: 532 (or a variant thereof). In some such compositions, the anti-TfR antigen binding protein comprises: (i) a HCVR that comprises the amino acid sequence set forth in SEQ ID NO: 437 (or a variant thereof); and a LCVR that comprises the amino acid sequence set forth in SEQ ID NO: 442 (or a variant thereof); or (ii) a HCVR that comprises the amino acid sequence set forth in SEQ ID NO: 457 (or a variant thereof); and a LCVR that comprises the amino acid sequence set forth in SEQ ID NO: 462 (or a variant thereof). In some such compositions, the TfR-binding delivery domain comprises an anti-TfR antibody, antibody fragment, or single-chain variable fragment (scFv). In some such compositions, the TfR-binding delivery domain is the single-chain variable fragment (scFv), optionally wherein the multidomain therapeutic protein comprises domains arranged in the following orientation: N′-Heavy chain variable region-Light chain variable region-lysosomal alpha-glucosidase-C′ or N′-Light chain variable region-Heavy chain variable region-lysosomal alpha-glucosidase-C′, optionally wherein the scFv and lysosomal alpha-glucosidase are connected by a peptide linker, and optionally wherein the peptide linker which is -(GGGGS)ₘ-(SEQ ID NO: 600); wherein m is 1, 2, 3, 4, 5, 6, 7, 8, 9, or 10, optionally wherein the scFv variable regions are connected by a peptide linker, and optionally wherein the peptide linker which is -(GGGGS)ₘ- (SEQ ID NO: 600); wherein m is 1, 2, 3, 4, 5, 6, 7, 8, 9, or 10. In some such compositions, the multidomain therapeutic protein comprises a heavy chain variable region (V_{H}) and a light chain variable region (V_{L}), and a lysosomal alpha-glucosidase, wherein the V_{H}, V_{L} and lysosomal alpha-glucosidase are arranged as follows: (i) V_{L}-V_{H}-lysosomal alpha-glucosidase; (ii) V_{H}-V_{L}-lysosomal alpha-glucosidase; (iii) V_{L}-[(GGGGS)₃]-V_{H}-[(GGGGS)₂]-lysosomal alpha-glucosidase; or (iv) V_{H}-[(GGGGS)₃]-V_{L}-[(GGGGS)₂]-lysosomal alpha-glucosidase. In some such compositions, the scFv comprises the sequence set forth in any one of SEQ ID NOS: 540, 549, 551, and 554, optionally wherein the scFv comprises the sequence set forth in SEQ ID NO: 554. In some such compositions, the scFv consists of the sequence set forth in any one of SEQ ID NOS: 540, 549, 551, and 554, optionally wherein the scFv consists of the sequence set forth in SEQ ID NOS: 554. In some such compositions, the scFv coding sequence is at least 90%, at least 91%, at least 92%, at least 93%, at least 94%, at least 95%, at least 96%, at least 97%, at least 98%, or at least 99% identical to any one of SEQ ID NOS: 587-599. Optionally, the scFv coding sequence is at least 90%, at least 91%, at least 92%, at least 93%, at least 94%, at least 95%, at least 96%, at least 97%, at least 98%, or at least 99% identical to any one of SEQ ID NOS: 593-595, optionally wherein the scFv coding sequence is at least 90%, at least 91%, at least 92%, at least 93%, at least 94%, at least 95%, at least 96%, at least 97%, at least 98%, or at least 99% identical to SEQ ID NO: 593. Optionally, the scFv coding sequence is at least 90%, at least 91%, at least 92%, at least 93%, at least 94%, at least 95%, at least 96%, at least 97%, at least 98%, or at least 99% identical to any one of SEQ ID NOS: 590-592, optionally wherein the scFv coding sequence is at least 90%, at least 91%, at least 92%, at least 93%, at least 94%, at least 95%, at least 96%, at least 97%, at least 98%, or at least 99% identical to SEQ ID NO: 590. In some such compositions, the scFv coding sequence is at least 90%, at least 91%, at least 92%, at least 93%, at least 94%, at least 95%, at least 96%, at least 97%, at least 98%, or at least 99% identical to any one of SEQ ID NOS: 587-599 and encodes an scFv comprising any one of SEQ ID NOS: 540, 549, 551, and 554. Optionally, wherein the scFv coding sequence is at least 90%, at least 91%, at least 92%, at least 93%, at least 94%, at least 95%, at least 96%, at least 97%, at least 98%, or at least 99% identical to any one of SEQ ID NOS: 593-595 and encodes an scFv comprising SEQ ID NO: 554, optionally wherein the scFv coding sequence is at least 90%, at least 91%, at least 92%, at least 93%, at least 94%, at least 95%, at least 96%, at least 97%, at least 98%, or at least 99% identical to SEQ ID NO: 593 and encodes an scFv comprising SEQ ID NO: 554. Optionally, wherein the scFv coding sequence is at least 90%, at least 91%, at least 92%, at least 93%, at least 94%, at least 95%, at least 96%, at least 97%, at least 98%, or at least 99% identical to any one of SEQ ID NOS: 590-592 and encodes an scFv comprising SEQ ID NO: 551, optionally wherein the scFv coding sequence is at least 90%, at least 91%, at least 92%, at least 93%, at least 94%, at least 95%, at least 96%, at least 97%, at least 98%, or at least 99% identical to SEQ ID NO: 590 and encodes an scFv comprising SEQ ID NO: 551. In some such compositions, the scFv coding sequence is at least 90%, at least 91%, at least 92%, at least 93%, at least 94%, at least 95%, at least 96%, at least 97%, at least 98%, or at least 99% identical to any one of SEQ ID NOS: 587-599, is codon-optimized and CpG-depleted, and encodes an scFv comprising any one of SEQ ID NOS: 540, 549, 551, and 554. Optionally, the scFv coding sequence is at least 90%, at least 91%, at least 92%, at least 93%, at least 94%, at least 95%, at least 96%, at least 97%, at least 98%, or at least 99% identical to any one of SEQ ID NOS: 593-595, is codon-optimized and CpG-depleted, and encodes an scFv comprising SEQ ID NO: 554, optionally wherein the scFv coding sequence is at least 90%, at least 91%, at least 92%, at least 93%, at least 94%, at least 95%, at least 96%, at least 97%, at least 98%, or at least 99% identical to SEQ ID NO: 593, the scFv coding sequence is codon-optimized and CpG-depleted, and encodes an scFv comprising SEQ ID NO: 554. Optionally, the scFv coding sequence is at least 90%, at least 91%, at least 92%, at least 93%, at least 94%, at least 95%, at least 96%, at least 97%, at least 98%, or at least 99% identical to any one of SEQ ID NOS: 590-592, is codon-optimized and CpG-depleted, and encodes an scFv comprising SEQ ID NO: 551, optionally wherein the scFv coding sequence is at least 90%, at least 91%, at least 92%, at least 93%, at least 94%, at least 95%, at least 96%, at least 97%, at least 98%, or at least 99% identical to SEQ ID NO: 590, the scFv coding sequence is codon-optimized and CpG-depleted, and encodes an scFv comprising SEQ ID NO: 551. In some such compositions, the scFv coding sequence comprises the sequence set forth in any one of SEQ ID NOS: 587-599. Optionally, the scFv coding sequence comprises the sequence set forth in any one of SEQ ID NOS: 593-595, optionally wherein the scFv coding sequence comprises the sequence set forth in SEQ ID NO: 593. Optionally, the scFv coding sequence comprises the sequence set forth in any one of SEQ ID NOS: 590-592, optionally wherein the scFv coding sequence comprises the sequence set forth in SEQ ID NO: 590. In some such compositions, the scFv coding sequence consists of the sequence set forth in any one of SEQ ID NOS: 587-599. Optionally, the scFv coding sequence consists of the sequence set forth in any one of SEQ ID NOS: 593-595, optionally wherein the scFv coding sequence consists of the sequence set forth in SEQ ID NO: 593. Optionally, the scFv coding sequence consists of the sequence set forth in any one of SEQ ID NOS: 590-592, optionally wherein the scFv coding sequence consists of the sequence set forth in SEQ ID NO: 590.

In some such compositions, the coding sequence for the multidomain therapeutic protein is codon-optimized or CpG-depleted. In some such compositions, the coding sequence for the multidomain therapeutic protein is codon-optimized and CpG-depleted. In some such compositions, the multidomain therapeutic protein comprises the sequence set forth in any one of SEQ ID NOS: 570-573, optionally wherein the multidomain therapeutic protein comprises the sequence set forth in SEQ ID NO: 573. In some such compositions, the multidomain therapeutic protein consists of the sequence set forth in any one of SEQ ID NOS: 570-573, optionally wherein the multidomain therapeutic protein consists of the sequence set forth in SEQ ID NO: 573. In some such compositions, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 574-586. Optionally, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 584-586, optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 584, optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 733. Optionally, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 581-583, optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 581, optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 729. In some such compositions, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 574-586, and the multidomain therapeutic protein comprises the sequence set forth in any one of SEQ ID NOS: 570-573. Optionally, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 584-586, and the multidomain therapeutic protein comprises the sequence set forth in SEQ ID NO: 573, optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 584, and the multidomain therapeutic protein comprises the sequence set forth in SEQ ID NO: 573, optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 733, and the multidomain therapeutic protein comprises the sequence set forth in SEQ ID NO: 573. Optionally, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 581-583, and the multidomain therapeutic protein comprises the sequence set forth in SEQ ID NO: 572, optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 581, and the multidomain therapeutic protein comprises the sequence set forth in SEQ ID NO: 572, optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 729, and the multidomain therapeutic protein comprises the sequence set forth in SEQ ID NO: 572. In some such compositions, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 574-586 and is codon-optimized and CpG-depleted, and the multidomain therapeutic protein comprises the sequence set forth in any one of SEQ ID NOS: 570-573. Optionally, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 584-586 and is codon-optimized and CpG-depleted, and the multidomain therapeutic protein comprises the sequence set forth in SEQ ID NO: 573, optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 584, the coding sequence for the multidomain therapeutic protein is codon-optimized and CpG-depleted, and the multidomain therapeutic protein comprises the sequence set forth in SEQ ID NO: 573, optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 733, the coding sequence for the multidomain therapeutic protein is codon-optimized and CpG-depleted, and the multidomain therapeutic protein comprises the sequence set forth in SEQ ID NO: 573. Optionally, the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 581-583 and is codon-optimized and CpG-depleted, and the multidomain therapeutic protein comprises the sequence set forth in SEQ ID NO: 572, optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 581, the coding sequence for the multidomain therapeutic protein is codon-optimized and CpG-depleted, and the multidomain therapeutic protein comprises the sequence set forth in SEQ ID NO: 572, optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 729, the coding sequence for the multidomain therapeutic protein is codon-optimized and CpG-depleted, and the multidomain therapeutic protein comprises the sequence set forth in SEQ ID NO: 572. In some such compositions, the coding sequence for the multidomain therapeutic protein comprises the sequence set forth in any one of SEQ ID NOS: 574-586. Optionally, the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733. Optionally, the coding sequence for the multidomain therapeutic protein comprises the sequence set forth in any one of SEQ ID NOS: 581-583, optionally wherein the coding sequence for the multidomain therapeutic protein comprises the sequence set forth in SEQ ID NO: 581, optionally wherein the nucleic acid construct comprises the sequence set forth in SEQ ID NO: 729. In some such compositions, the coding sequence for the multidomain therapeutic protein consists of the sequence set forth in any one of SEQ ID NOS: 574-586. Optionally, the coding sequence for the multidomain therapeutic protein consists of the sequence set forth in any one of SEQ ID NOS: 584-586, optionally wherein the coding sequence for the multidomain therapeutic protein consists of the sequence set forth in SEQ ID NO: 584, optionally wherein the nucleic acid construct comprises the sequence set forth in SEQ ID NO: 733. Optionally, the coding sequence for the multidomain therapeutic protein consists of the sequence set forth in any one of SEQ ID NOS: 581-583, optionally wherein the coding sequence for the multidomain therapeutic protein consists of the sequence set forth in SEQ ID NO: 581, optionally wherein the nucleic acid construct comprises the sequence set forth in SEQ ID NO: 729. In some such compositions, the nucleic acid construct comprises a splice acceptor upstream of the coding sequence for the multidomain therapeutic protein. In some such compositions, the nucleic acid construct comprises a polyadenylation signal or sequence downstream of the coding sequence for the multidomain therapeutic protein. In some such compositions, the nucleic acid construct comprises a splice acceptor upstream of the coding sequence for the multidomain therapeutic protein, and the nucleic acid construct comprises a polyadenylation signal or sequence downstream of the coding sequence for the multidomain therapeutic protein. In some such compositions, the nucleic acid construct does not comprise a homology arm. In some such compositions, the nucleic acid construct comprises homology arms. In some such compositions, the nucleic acid construct does not comprise a promoter that drives the expression of the multidomain therapeutic protein. In some such compositions, the coding sequence for the multidomain therapeutic protein is operably linked to a promoter, optionally wherein the promoter is a liver-specific promoter. In some such compositions, the nucleic acid construct is single-stranded DNA or double-stranded DNA. In some such compositions, the nucleic acid construct is single-stranded DNA. In some such compositions, the nucleic acid construct comprises from 5′ to 3′: a splice acceptor, the coding sequence for the multidomain therapeutic protein, and a polyadenylation signal or sequence, wherein the lysosomal alpha-glucosidase coding sequence comprises the sequence set forth in any one of SEQ ID NOS: 174-182 and 205-212, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733, or optionally wherein the coding sequence for the multidomain therapeutic protein comprises the sequence set forth in any one of SEQ ID NOS: 581-583, optionally wherein the coding sequence for the multidomain therapeutic protein comprises the sequence set forth in SEQ ID NO: 581, optionally wherein the nucleic acid construct comprises the sequence set forth in SEQ ID NO: 729, wherein the nucleic acid construct does not comprise a promoter that drives the expression of the multidomain therapeutic protein, and wherein the nucleic acid construct does not comprise a homology arm. In some such compositions, the nucleic acid construct comprises from 5′ to 3′: a splice acceptor, the coding sequence for the multidomain therapeutic protein, and a polyadenylation signal or sequence, wherein the lysosomal alpha-glucosidase coding sequence comprises the sequence set forth in any one of SEQ ID NOS: 174-182, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733, or optionally wherein the coding sequence for the multidomain therapeutic protein comprises the sequence set forth in any one of SEQ ID NOS: 581-583, optionally wherein the coding sequence for the multidomain therapeutic protein comprises the sequence set forth in SEQ ID NO: 581, optionally wherein the nucleic acid construct comprises the sequence set forth in SEQ ID NO: 729, wherein the nucleic acid construct does not comprise a promoter that drives the expression of the multidomain therapeutic protein, and wherein the nucleic acid construct does not comprise a homology arm.

In some such compositions, the nucleic acid construct is in a nucleic acid vector or a lipid nanoparticle. In some such compositions, the nucleic acid construct is in the nucleic acid vector. In some such compositions, the nucleic acid vector is a viral vector. In some such compositions, the nucleic acid vector is an adeno-associated viral (AAV) vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160. In some such compositions, the AAV vector is a single-stranded AAV (ssAAV) vector. In some such compositions, the AAV vector is derived from an AAV8 vector, an AAV3B vector, an AAV5 vector, an AAV6 vector, an AAV7 vector, an AAV9 vector, an AAVrh.74 vector, or an AAVhu.37 vector. In some such compositions, the AAV vector is a recombinant AAV8 (rAAV8) vector. In some such compositions, the AAV vector is a single-stranded rAAV8 vector. In some such compositions, the nucleic acid construct comprises from 5′ to 3′: a splice acceptor, the coding sequence for the multidomain therapeutic protein, and a polyadenylation signal or sequence, wherein the lysosomal alpha-glucosidase coding sequence comprises the sequence set forth in any one of SEQ ID NOS: 174-182 and 205-212, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733, or optionally wherein the coding sequence for the multidomain therapeutic protein comprises the sequence set forth in any one of SEQ ID NOS: 581-583, optionally wherein the coding sequence for the multidomain therapeutic protein comprises the sequence set forth in SEQ ID NO: 581, optionally wherein the nucleic acid construct comprises the sequence set forth in SEQ ID NO: 729, wherein the nucleic acid construct does not comprise a promoter that drives the expression of the multidomain therapeutic protein, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160. In some such compositions, the nucleic acid construct comprises from 5′ to 3′: a splice acceptor, the coding sequence for the multidomain therapeutic protein, and a polyadenylation signal or sequence, wherein the lysosomal alpha-glucosidase coding sequence comprises the sequence set forth in any one of SEQ ID NOS: 174-182, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733, or optionally wherein the coding sequence for the multidomain therapeutic protein comprises the sequence set forth in any one of SEQ ID NOS: 581-583, optionally wherein the coding sequence for the multidomain therapeutic protein comprises the sequence set forth in SEQ ID NO: 581, optionally wherein the nucleic acid construct comprises the sequence set forth in SEQ ID NO: 729, wherein the nucleic acid construct does not comprise a promoter that drives the expression of the multidomain therapeutic protein, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160. In some such compositions, the nucleic acid construct is CpG-depleted.

In some such compositions, the composition further comprises a nuclease agent that targets a nuclease target site in a target genomic locus. In some such compositions, the target genomic locus is an albumin gene, optionally wherein the albumin gene is a human albumin gene. In some such compositions, the nuclease target site is in intron 1 of the albumin gene. In some such compositions, the nuclease agent comprises: (a) a zinc finger nuclease (ZFN); (b) a transcription activator-like effector nuclease (TALEN); or (c) (i) a Cas protein or a nucleic acid encoding the Cas protein; and (ii) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

In some such compositions, the nuclease agent comprises: (a) a Cas protein or a nucleic acid encoding the Cas protein; and (b) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence. In some such compositions, the guide RNA target sequence is in intron 1 of an albumin gene. In some such compositions, the albumin gene is a human albumin gene. In some such compositions, the DNA-targeting segment comprises at least 17, at least 18, at least 19, or at least 20 contiguous nucleotides of the sequence set forth in any one of SEQ ID NOS: 30-61, optionally wherein the DNA-targeting segment comprises at least 17, at least 18, at least 19, or at least 20 contiguous nucleotides of the sequence set forth in any one of SEQ ID NOS: 36, 30, 33, and 41. In some such compositions, the DNA-targeting segment is at least 90% or at least 95% identical to the sequence set forth in any one of SEQ ID NOS: 30-61, optionally wherein the DNA-targeting segment is at least 90% or at least 95% identical to the sequence set forth in any one of SEQ ID NOS: 36, 30, 33, and 41. In some such compositions, the DNA-targeting segment comprises any one of SEQ ID NOS: 30-61, optionally wherein the DNA-targeting segment comprises any one of SEQ ID NOS: 36, 30, 33, and 41. In some such compositions, the DNA-targeting segment consists of any one of SEQ ID NOS: 30-61, optionally wherein the DNA-targeting segment consists of any one of SEQ ID NOS: 36, 30, 33, and 41. In some such compositions, the guide RNA comprises any one of SEQ ID NOS: 62-125, optionally wherein the guide RNA comprises any one of SEQ ID NOS: 68, 100, 62, 94, 65, 97, 73, and 105. In some such compositions, the DNA-targeting segment comprises at least 17, at least 18, at least 19, or at least 20 contiguous nucleotides of SEQ ID NO: 36. In some such compositions, the DNA-targeting segment is at least 90% or at least 95% identical to SEQ ID NO: 36. In some such compositions, the DNA-targeting segment comprises SEQ ID NO: 36. In some such compositions, the DNA-targeting segment consists of SEQ ID NO: 36. In some such compositions, the guide RNA comprises SEQ ID NO: 68 or 100.

In some such compositions, the guide RNA in the form of RNA. In some such compositions, the guide RNA comprises at least one modification. In some such compositions, the at least one modification comprises a 2′-O-methyl-modified nucleotide. In some such compositions, the at least one modification comprises a phosphorothioate bond between nucleotides. In some such compositions, the at least one modification comprises a modification at one or more of the first five nucleotides at the 5′ end of the guide RNA. In some such compositions, the at least one modification comprises a modification at one or more of the last five nucleotides at the 3′ end of the guide RNA. In some such compositions, the at least one modification comprises phosphorothioate bonds between the first four nucleotides at the 5′ end of the guide RNA. In some such compositions, the at least one modification comprises phosphorothioate bonds between the last four nucleotides at the 3′ end of the guide RNA. In some such compositions, the at least one modification comprises 2′-O-methyl-modified nucleotides at the first three nucleotides at the 5′ end of the guide RNA. In some such compositions, the at least one modification comprises 2′-O-methyl-modified nucleotides at the last three nucleotides at the 3′ end of the guide RNA. In some such compositions, the at least one modification comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA. In some such compositions, the guide RNA is a single guide RNA (sgRNA). In some such compositions, the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

In some such compositions, the Cas protein is a Cas9 protein. In some such compositions, the Cas9 protein is derived from a Streptococcus pyogenes Cas9 protein, a Staphylococcus aureus Cas9 protein, a Campylobacter jejuni Cas9 protein, a Streptococcus thermophilus Cas9 protein, or a Neisseria meningitidis Cas9 protein. In some such compositions, the Cas protein is derived from a Streptococcus pyogenes Cas9 protein. In some such compositions, the Cas protein comprises the sequence set forth in SEQ ID NO: 11. In some such compositions, the nucleic acid encoding the Cas protein is codon-optimized for expression in a mammalian cell or a human cell. In some such compositions, the composition comprises the nucleic acid encoding the Cas protein, wherein the nucleic acid comprises an mRNA encoding the Cas protein. In some such compositions, the mRNA encoding the Cas protein comprises at least one modification. In some such compositions, the mRNA encoding the Cas protein is modified to comprise a modified uridine at one or more or all uridine positions. In some such compositions, the modified uridine is pseudouridine or N1-methyl-pseudouridine, optionally N1-methyl-pseudouridine. In some such compositions, the mRNA encoding the Cas protein is fully substituted with pseudouridine or N1-methyl-pseudouridine, optionally N1-methyl-pseudouridine. In some such compositions, the modified uridine is pseudouridine. In some such compositions, the mRNA encoding the Cas protein is fully substituted with pseudouridine. In some such compositions, the mRNA encoding the Cas protein comprises a 5′ cap. In some such compositions, the mRNA encoding the Cas protein comprises a polyadenylation sequence. In some such compositions, the mRNA encoding the Cas protein comprises the sequence set forth in SEQ ID NO: 226, 225, or 12. In some such compositions, the composition comprises the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine or N1-methyl-pseudouridine, optionally N1-methyl-pseudouridine, comprises a 5′ cap, and comprises a polyadenylation sequence. In some such compositions, the composition comprises the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine, comprises a 5′ cap, and comprises a polyadenylation sequence.

In some such compositions, the guide RNA in the form of RNA, and the guide RNA comprises SEQ ID NO: 68 or 100, and wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 226, 225, or 12. In some such compositions, the nucleic acid construct comprises from 5′ to 3′: a splice acceptor, the coding sequence for the multidomain therapeutic protein, and a polyadenylation signal or sequence, wherein the lysosomal alpha-glucosidase coding sequence comprises the sequence set forth in any one of SEQ ID NOS: 174-182 and 205-212, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733, or optionally wherein the coding sequence for the multidomain therapeutic protein comprises the sequence set forth in any one of SEQ ID NOS: 581-583, optionally wherein the coding sequence for the multidomain therapeutic protein comprises the sequence set forth in SEQ ID NO: 581, optionally wherein the nucleic acid construct comprises the sequence set forth in SEQ ID NO: 729, wherein the nucleic acid construct does not comprise a promoter that drives the expression of the multidomain therapeutic protein, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160. In some such compositions, the nucleic acid construct comprises from 5′ to 3′: a splice acceptor, the coding sequence for the multidomain therapeutic protein, and a polyadenylation signal or sequence, wherein the lysosomal alpha-glucosidase coding sequence comprises the sequence set forth in any one of SEQ ID NOS: 174-182, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733, or optionally wherein the coding sequence for the multidomain therapeutic protein comprises the sequence set forth in any one of SEQ ID NOS: 581-583, optionally wherein the coding sequence for the multidomain therapeutic protein comprises the sequence set forth in SEQ ID NO: 581, optionally wherein the nucleic acid construct comprises the sequence set forth in SEQ ID NO: 729, wherein the nucleic acid construct does not comprise a promoter that drives the expression of the multidomain therapeutic protein, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160. In some such compositions, the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine or N1-methyl-pseudouridine, optionally N1-methyl-pseudouridine, comprises a 5′ cap, and comprises a polyadenylation sequence. In some such compositions, the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine, comprises a 5′ cap, and comprises a polyadenylation sequence. In some such compositions, the nucleic acid construct comprises from 5′ to 3′: a splice acceptor, the coding sequence for the multidomain therapeutic protein, and a polyadenylation signal or sequence, wherein the lysosomal alpha-glucosidase coding sequence comprises the sequence set forth in any one of SEQ ID NOS: 174-182 and 205-212, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733, or optionally wherein the coding sequence for the multidomain therapeutic protein comprises the sequence set forth in any one of SEQ ID NOS: 581-583, optionally wherein the coding sequence for the multidomain therapeutic protein comprises the sequence set forth in SEQ ID NO: 581, optionally wherein the nucleic acid construct comprises the sequence set forth in SEQ ID NO: 729, wherein the nucleic acid construct does not comprise a promoter that drives the expression of the multidomain therapeutic protein, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160. In some such compositions, the nucleic acid construct comprises from 5′ to 3′: a splice acceptor, the coding sequence for the multidomain therapeutic protein, and a polyadenylation signal or sequence, wherein the lysosomal alpha-glucosidase coding sequence comprises the sequence set forth in any one of SEQ ID NOS: 174-182, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733, or optionally wherein the coding sequence for the multidomain therapeutic protein comprises the sequence set forth in any one of SEQ ID NOS: 581-583, optionally wherein the coding sequence for the multidomain therapeutic protein comprises the sequence set forth in SEQ ID NO: 581, optionally wherein the nucleic acid construct comprises the sequence set forth in SEQ ID NO: 729, wherein the nucleic acid construct does not comprise a promoter that drives the expression of the multidomain therapeutic protein, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

In some such compositions, the Cas protein or the nucleic acid encoding the Cas protein and the guide RNA or the one or more DNAs encoding the guide RNA are associated with a lipid nanoparticle. In some such compositions, the lipid nanoparticle comprises a cationic lipid, a neutral lipid, a helper lipid, and a stealth lipid. In some such compositions, the cationic lipid is Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate). In some such compositions, the neutral lipid is distearoylphosphatidylcholine or 1,2-distearoyl-sn-glycero-3-phosphocholine (DSPC). In some such compositions, the helper lipid is cholesterol. In some such compositions, the stealth lipid is PEG2k-DMG. In some such compositions, the cationic lipid is Lipid A, the neutral lipid is DSPC, the helper lipid is cholesterol, and the stealth lipid is PEG2k-DMG. In some such compositions, the lipid nanoparticle comprises four lipids at the following molar ratios: about 50 mol% Lipid A, about 9 mol% DSPC, about 38 mol% cholesterol, and about 3 mol% PEG2k-DMG.

In some such compositions, the albumin gene is a human albumin gene, wherein the guide RNA in the form of RNA, and the guide RNA comprises SEQ ID NO: 68 or 100, wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 226, 225, or 12, and wherein the guide RNA and the mRNA encoding the Cas protein are associated with a lipid nanoparticle comprising Lipid A, DSPC, cholesterol, and PEG2k-DMG, optionally at the following molar ratios: about 50 mol% Lipid A, about 9 mol% DSPC, about 38 mol% cholesterol, and about 3 mol% PEG2k-DMG. In some such compositions, the nucleic acid construct comprises from 5′ to 3′: a splice acceptor, the coding sequence for the multidomain therapeutic protein, and a polyadenylation signal or sequence, wherein the lysosomal alpha-glucosidase coding sequence comprises the sequence set forth in any one of SEQ ID NOS: 174-182 and 205-212, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733, or optionally wherein the coding sequence for the multidomain therapeutic protein comprises the sequence set forth in any one of SEQ ID NOS: 581-583, optionally wherein the coding sequence for the multidomain therapeutic protein comprises the sequence set forth in SEQ ID NO: 581, optionally wherein the nucleic acid construct comprises the sequence set forth in SEQ ID NO: 729, wherein the nucleic acid construct does not comprise a promoter that drives the expression of the multidomain therapeutic protein, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160. In some such compositions, the nucleic acid construct comprises from 5′ to 3′: a splice acceptor, the coding sequence for the multidomain therapeutic protein, and a polyadenylation signal or sequence, wherein the lysosomal alpha-glucosidase coding sequence comprises the sequence set forth in any one of SEQ ID NOS: 174-182, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733, or optionally wherein the coding sequence for the multidomain therapeutic protein comprises the sequence set forth in any one of SEQ ID NOS: 581-583, optionally wherein the coding sequence for the multidomain therapeutic protein comprises the sequence set forth in SEQ ID NO: 581, optionally wherein the nucleic acid construct comprises the sequence set forth in SEQ ID NO: 729, wherein the nucleic acid construct does not comprise a promoter that drives the expression of the multidomain therapeutic protein, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

In some such compositions, the albumin gene is a human albumin gene, wherein the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine or N1-methyl-pseudouridine, optionally N1-methyl-pseudouridine, comprises a 5′ cap, and comprises a polyadenylation sequence, and wherein the guide RNA and the mRNA encoding the Cas protein are associated with a lipid nanoparticle comprising Lipid A, DSPC, cholesterol, and PEG2k-DMG, optionally at the following molar ratios: about 50 mol% Lipid A, about 9 mol% DSPC, about 38 mol% cholesterol, and about 3 mol% PEG2k-DMG. In some such compositions, the albumin gene is a human albumin gene, wherein the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine, comprises a 5′ cap, and comprises a polyadenylation sequence, and wherein the guide RNA and the mRNA encoding the Cas protein are associated with a lipid nanoparticle comprising Lipid A, DSPC, cholesterol, and PEG2k-DMG, optionally at the following molar ratios: about 50 mol% Lipid A, about 9 mol% DSPC, about 38 mol% cholesterol, and about 3 mol% PEG2k-DMG. In some such compositions, the nucleic acid construct comprises from 5′ to 3′: a splice acceptor, the coding sequence for the multidomain therapeutic protein, and a polyadenylation signal or sequence, wherein the lysosomal alpha-glucosidase coding sequence comprises the sequence set forth in any one of SEQ ID NOS: 174-182 and 205-212, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733, or optionally wherein the coding sequence for the multidomain therapeutic protein comprises the sequence set forth in any one of SEQ ID NOS: 581-583, optionally wherein the coding sequence for the multidomain therapeutic protein comprises the sequence set forth in SEQ ID NO: 581, optionally wherein the nucleic acid construct comprises the sequence set forth in SEQ ID NO: 729, wherein the nucleic acid construct does not comprise a promoter that drives the expression of the multidomain therapeutic protein, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160. In some such compositions, the nucleic acid construct comprises from 5′ to 3′: a splice acceptor, the coding sequence for the multidomain therapeutic protein, and a polyadenylation signal or sequence, wherein the lysosomal alpha-glucosidase coding sequence comprises the sequence set forth in any one of SEQ ID NOS: 174-182, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733, or optionally wherein the coding sequence for the multidomain therapeutic protein comprises the sequence set forth in any one of SEQ ID NOS: 581-583, optionally wherein the coding sequence for the multidomain therapeutic protein comprises the sequence set forth in SEQ ID NO: 581, optionally wherein the nucleic acid construct comprises the sequence set forth in SEQ ID NO: 729, wherein the nucleic acid construct does not comprise a promoter that drives the expression of the multidomain therapeutic protein, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

In some such compositions, the composition is for use in a method of inserting the nucleic acid encoding the multidomain therapeutic protein into a target genomic locus in a cell or a population of cells. In some such compositions, the composition is for use in a method of expressing the multidomain therapeutic protein from a target genomic locus in a cell or a population of cells. In some such compositions, the composition is for use in a method of expressing the multidomain therapeutic protein in a cell or a population of cells. In some such compositions, the composition is for use in a method of inserting the nucleic acid encoding the multidomain therapeutic protein into a target genomic locus in a cell or a population of cells in a subject. In some such compositions, the composition is for use in a method of expressing the multidomain therapeutic protein from a target genomic locus in a cell or a population of cells in a subject. In some such compositions, the composition is for use in a method of expressing the multidomain therapeutic protein in a cell or a population of cells in a subject. In some such compositions, the cell is a neonatal cell and the population of cells is a population of neonatal cells. In some such compositions, the neonatal cell or the population of neonatal cells is from a human neonatal subject within 24 weeks after birth. In some such compositions, the neonatal cell or the population of neonatal cells is from a human neonatal subject within 12 weeks after birth. In some such compositions, the neonatal cell or the population of neonatal cells is from a human neonatal subject within 8 weeks after birth. In some such compositions, the neonatal cell or the population of neonatal cells is from a human neonatal subject within 4 weeks after birth. In some such compositions, the cell is a not a neonatal cell and the population of cells is not a population of neonatal cells. In some such compositions, the composition is for use in a method of treating a lysosomal alpha-glucosidase deficiency in a subject in need thereof. In some such compositions, the composition is for use in a method of reducing glycogen accumulation in a tissue in a subject in need thereof. In some such compositions, the composition is for use in a method of treating Pompe disease in a subject in need thereof. In some such compositions, the composition is for use in a method of preventing or reducing the onset of a sign or symptom of Pompe disease in a neonatal subject in need thereof. In some such compositions, the subject is a neonatal subject. In some such compositions, the neonatal subject is a human neonatal subject within 24 weeks after birth. In some such compositions, the neonatal subject is a human neonatal subject within 12 weeks after birth. In some such compositions, the neonatal subject is a human neonatal subject within 8 weeks after birth. In some such compositions, the neonatal subject is a human neonatal subject within 4 weeks after birth. In some such compositions, the subject is not a neonatal subject.

In another aspect, provided is a cell comprising any of the above compositions. In some such cells, the nucleic acid construct is integrated into a target genomic locus, and wherein the multidomain therapeutic protein is expressed from the target genomic locus, or wherein the nucleic acid construct is integrated into intron 1 of an endogenous albumin locus, and wherein the multidomain therapeutic protein is expressed from the endogenous albumin locus. In some such cells, the cell is a human cell. In some such cells, the cell is a liver cell. In some such cells, the liver cell is a hepatocyte. In some such cells, the cell is a neonatal cell. In some such cells, the neonatal cell is from a human neonatal subject within 24 weeks after birth. In some such cells, the neonatal cell is from a human neonatal subject within 12 weeks after birth. In some such cells, the neonatal cell is from a human neonatal subject within 8 weeks after birth. In some such cells, the neonatal cell is from a human neonatal subject within 4 weeks after birth. In some such cells, the cell is not a neonatal cell. In some such cells, the cell is in vivo. In some such cells, the cell is in vitro or ex vivo.

In another aspect, provided are methods of inserting a nucleic acid encoding a multidomain therapeutic protein comprising a TfR-binding delivery domain fused to a lysosomal alpha-glucosidase into a target genomic locus in a cell or a population of cells. Some such methods comprise administering to the cell or the population of cells any of the above compositions, wherein the nuclease agent cleaves the nuclease target site in the target genomic locus, and the nucleic acid construct is inserted into the target genomic locus. In another aspect, provided are methods of expressing a multidomain therapeutic protein comprising a TfR-binding delivery domain fused to a lysosomal alpha-glucosidase in a cell or a population of cells. Some such methods comprise administering to the cell or the population of cells any of the above compositions, wherein the coding sequence for the multidomain therapeutic protein is operably linked to a promoter in the nucleic acid construct and is expressed in the cell or population of cells. In another aspect, provided are methods of expressing a multidomain therapeutic protein comprising a TfR-binding delivery domain fused to a lysosomal alpha-glucosidase from a target genomic locus in a cell or a population of cells. Some such methods comprise administering to the cell or the population of cells any of the above compositions, wherein the nuclease agent cleaves the nuclease target site in the target genomic locus, the nucleic acid construct is inserted into the target genomic locus to create a modified target genomic locus, and the multidomain therapeutic protein comprising the TfR-binding delivery domain fused to the lysosomal alpha-glucosidase is expressed from the modified target genomic locus. In some such methods, the cell is a liver cell or the population of cells is a population of liver cells. In some such methods, the cell is a hepatocyte or the population of cells is a population of hepatocytes. In some such methods, the cell is a human cell or the population of cells is a population of human cells. In some such methods, the cell is a neonatal cell or the population of cells is a population of neonatal cells. In some such methods, the neonatal cell or the population of neonatal cells is from a human neonatal subject within 24 weeks after birth. In some such methods, the neonatal cell or the population of neonatal cells is from a human neonatal subject within 12 weeks after birth. In some such methods, the neonatal cell or the population of neonatal cells is from a human neonatal subject within 8 weeks after birth. In some such methods, the neonatal cell or the population of neonatal cells is from a human neonatal subject within 4 weeks after birth. In some such methods, the cell is not a neonatal cell or the population of cells is not a population of neonatal cells. In some such methods, the cell is in vitro or ex vivo or the population of cells is in vitro or ex vivo. In some such methods, the cell is in vivo in a subject or the population of cells is in vivo in a subject.

In another aspect, provided are methods of inserting a nucleic acid encoding a multidomain therapeutic protein comprising a TfR-binding delivery domain fused to a lysosomal alpha-glucosidase into a target genomic locus in a cell or a population of cells in a subject. Some such methods comprise administering to the subject any of the above compositions, wherein the nuclease agent cleaves the nuclease target site in the target genomic locus, and the nucleic acid construct is inserted into the target genomic locus. In another aspect, provided are methods of expressing a multidomain therapeutic protein comprising a TfR-binding delivery domain fused to a lysosomal alpha-glucosidase protein in a cell or a population of cells in a subject. Some such methods comprise administering to the subject any of the above compositions, wherein the coding sequence for the multidomain therapeutic protein is operably linked to a promoter in the nucleic acid construct and is expressed in the cell. In another aspect, provided are methods of expressing a multidomain therapeutic protein comprising a TfR-binding delivery domain fused to a lysosomal alpha-glucosidase protein from a target genomic locus in a cell or a population of cells in a subject. Some such methods comprise administering to the subject any of the above compositions, wherein the nuclease agent cleaves the nuclease target site in the target genomic locus, the nucleic acid construct is inserted into the target genomic locus to create a modified target genomic locus, and the multidomain therapeutic protein comprising the TfR-binding delivery domain fused to the lysosomal alpha-glucosidase is expressed from the modified target genomic locus. In some such methods, the expressed multidomain therapeutic protein is delivered to and internalized by skeletal muscle, heart, and central nervous system tissue in the subject. In some such methods, the cell is a liver cell or the population of cells is a population of liver cells. In some such methods, the cell is a hepatocyte or the population of cells is a population of hepatocytes. In some such methods, the cell is a human cell or the population of cells is a population of human cells. In some such methods, the cell is a neonatal cell or the population of cells is a population of neonatal cells. In some such methods, the neonatal cell or the population of neonatal cells is from a human neonatal subject within 24 weeks after birth. In some such methods, the neonatal cell or the population of neonatal cells is from a human neonatal subject within 12 weeks after birth. In some such methods, the neonatal cell or the population of neonatal cells is from a human neonatal subject within 8 weeks after birth. In some such methods, the neonatal cell or the population of neonatal cells is from a human neonatal subject within 4 weeks after birth. In some such methods, the cell is not a neonatal cell or the population of cells is not a population of neonatal cells.

In another aspect, provided are methods of treating a lysosomal alpha-glucosidase deficiency in a subject in need thereof. Some such methods comprise administering to the subject any of the above compositions, wherein the coding sequence for the multidomain therapeutic protein is operably linked to a promoter in the nucleic acid construct and is expressed in the subject. Some such methods comprise administering to the subject any of the above compositions, wherein the nuclease agent cleaves the nuclease target site in the target genomic locus, the nucleic acid construct is inserted into the target genomic locus to create a modified target genomic locus, and the multidomain therapeutic protein comprising the TfR-binding delivery domain fused to the lysosomal alpha-glucosidase is expressed from the modified target genomic locus. In another aspect, provided are methods of reducing glycogen accumulation in a tissue in a subject in need thereof. Some such methods comprise administering to the subject any of the above compositions, wherein the coding sequence for the multidomain therapeutic protein is operably linked to a promoter in the nucleic acid construct and is expressed in the subject and reduces glycogen accumulation in the tissue. Some such methods comprise administering to the subject any of the above compositions, wherein the nuclease agent cleaves the nuclease target site in the target genomic locus, the nucleic acid construct is inserted into the target genomic locus to create a modified target genomic locus, and the multidomain therapeutic protein comprising the TfR-binding delivery domain fused to the lysosomal alpha-glucosidase is expressed from the modified target genomic locus and reduces glycogen accumulation in the tissue. In some such methods, the subject has Pompe disease. In another aspect, provided are methods of treating Pompe disease in a subject in need thereof. Some such methods comprise administering to the subject any of the above compositions, wherein the coding sequence for the multidomain therapeutic protein is operably linked to a promoter in the nucleic acid construct and is expressed in the subject, thereby treating the Pompe disease. Some such methods comprise administering to the subject any of the above compositions, wherein the nuclease agent cleaves the nuclease target site in the target genomic locus, the nucleic acid construct is inserted into the target genomic locus to create a modified target genomic locus, and the multidomain therapeutic protein comprising the TfR-binding delivery domain fused to the lysosomal alpha-glucosidase is expressed from the modified target genomic locus, thereby treating the Pompe disease. In some such methods, the Pompe disease is infantile-onset Pompe disease. In some such methods, the Pompe disease is late-onset Pompe disease.

In some such methods, the neonatal subject is a human subject within 24 weeks after birth. In some such methods, the neonatal subject is a human subject within 12 weeks after birth. In some such methods, the neonatal subject is a human subject within 8 weeks after birth. In some such methods, the neonatal subject is a human subject within 4 weeks after birth. In some such methods, the subject is not a neonatal subject.

In some such methods, the method results in a therapeutically effective level of circulating multidomain therapeutic protein or lysosomal alpha-glucosidase in the subject. In some such methods, the method reduces glycogen accumulation in skeletal muscle, heart, or central nervous system tissue in the subject. In some such methods, the method reduces glycogen accumulation in skeletal muscle, heart, and central nervous system tissue in the subject. In some such methods, the method results in reduced glycogen levels in skeletal muscle, heart, and/or central nervous system tissue in the subject comparable to wild type levels at the same age. In some such methods, the method improves muscle strength in the subject or prevents loss of muscle strength in the subject compared to a control subject. In some such methods, the method results in the subject having muscle strength comparable to wild type levels at the same age.

In another aspect, provided are methods of preventing or reducing the onset of a sign or symptom of Pompe disease in a neonatal subject in need thereof. Some such methods comprise administering to the neonatal subject any of the above compositions, wherein the coding sequence for the multidomain therapeutic protein is operably linked to a promoter in the nucleic acid construct and is expressed in the subject, thereby preventing or reducing the onset of a sign or symptom of the Pompe disease in the subject. Some such methods comprise administering to the neonatal subject any of the above compositions, wherein the nuclease agent cleaves the nuclease target site, the nucleic acid construct is inserted into the target genomic locus to create a modified target genomic locus, and the multidomain therapeutic protein comprising the TfR-binding delivery domain fused to the lysosomal alpha-glucosidase is expressed from the modified target genomic locus, thereby preventing or reducing the onset of a sign or symptom of the Pompe disease in the subject. In some such methods, the Pompe disease is infantile-onset Pompe disease. In some such methods, the Pompe disease is late-onset Pompe disease. In some such methods, the method results in a therapeutically effective level of circulating multidomain therapeutic protein or lysosomal alpha-glucosidase in the subject. In some such methods, the method prevents or reduces glycogen accumulation in skeletal muscle, heart, or central nervous system tissue in the subject. In some such methods, the method prevents or reduces glycogen accumulation in skeletal muscle, heart, and central nervous system tissue in the subject.

In some such methods, the subject is a neonatal subject. In some such methods, the neonatal subject is a human subject within 24 weeks after birth. In some such methods, the neonatal subject is a human subject within 12 weeks after birth. In some such methods, the neonatal subject is a human subject within 8 weeks after birth. In some such methods, the neonatal subject is a human subject within 4 weeks after birth. In some such methods, the subject is not a neonatal subject.

In some such methods, the method results in increased expression of the multidomain therapeutic protein in the subject compared to a method comprising administering an episomal expression vector encoding the multidomain therapeutic protein to a control subject. In some such methods, the method results in increased serum levels of the multidomain therapeutic protein in the subject compared to a method comprising administering an episomal expression vector encoding the multidomain therapeutic protein to a control subject. In some such methods, the method results in serum levels of the multidomain therapeutic protein in the subject of at least about 1 µg/mL, at least about 2 µg/mL, at least about 3 µg/mL, at least about 4 µg/mL, at least about 5 µg/mL, at least about 6 µg/mL, at least about 7 µg/mL, at least about 8 µg/mL, at least about 9 µg/mL, or at least about 10 µg/mL. In some such methods, the method results in serum levels of the multidomain therapeutic protein in the subject of at least about 2 µg/mL or at least about 5 µg/mL. In some such methods, the method results in serum levels of the multidomain therapeutic protein in the subject of between about 2 µg/mL and about 30 µg/mL or between about 2 µg/mL and about 20 µg/mL. In some such methods, the method results in serum levels of the multidomain therapeutic protein in the subject of between about 5 µg/mL and about 30 µg/mL or between about 5 µg/mL and about 20 µg/mL. In some such methods, the method achieves lysosomal alpha-glucosidase activity levels of at least about 40% of normal, at least about 45% of normal, at least about 50%, at least about 60%, at least about 70%, at least about 80%, at least about 90%, or 100% of normal. In some such methods, the subject has infantile-onset Pompe disease, and the method achieves lysosomal alpha-glucosidase expression or activity levels of at least about 1% or more than about 1% of normal. In some such methods, the subject has late-onset Pompe disease, and the method achieves lysosomal alpha-glucosidase expression or activity levels of at least about 40% of normal or more than about 40% of normal. In some such methods, the method increases lysosomal alpha-glucosidase activity over the subject’s baseline lysosomal alpha-glucosidase activity by at least about 1%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or at least about 100%. In some such methods, the expression or activity of the multidomain therapeutic protein is at least 50% of the expression or activity of the multidomain therapeutic protein at a peak level of expression measured for the subject at six months or 24 weeks after the administering. In some such methods, the expression or activity of the multidomain therapeutic protein is at least 50% of the expression or activity of the multidomain therapeutic protein at a peak level of expression measured for the subject at one year after the administering. In some such methods, the expression or activity of the multidomain therapeutic protein is at least 60% of the expression or activity of the multidomain therapeutic protein at a peak level of expression measured for the subject at six months or 24 weeks after the administering. In some such methods, the expression or activity of the multidomain therapeutic protein is at least 50% of the expression or activity of the multidomain therapeutic protein at a peak level of expression measured for the subject at two years after the administering. In some such methods, the expression or activity of the multidomain therapeutic protein is at least 60% of the expression or activity of the multidomain therapeutic protein at a peak level of expression measured for the subject at two years after the administering. In some such methods, the expression or activity of the multidomain therapeutic protein is at least 60% of the expression or activity of the multidomain therapeutic protein at a peak level of expression measured for the subject at six months or 24 weeks after the administering.

In some such methods, the method further comprises assessing preexisting AAV immunity in the subject prior to administering the nucleic acid construct to the subject. In some such methods, the preexisting AAV immunity is preexisting AAV8 immunity. In some such methods, assessing preexisting AAV immunity comprises assessing immunogenicity using a total antibody immune assay or a neutralizing antibody assay.

In some such methods, the nucleic acid construct is administered simultaneously with the nuclease agent or the one or more nucleic acids encoding the nuclease agent. In some such methods, the nucleic acid construct is not administered simultaneously with the nuclease agent or the one or more nucleic acids encoding the nuclease agent. In some such methods, the nucleic acid construct is administered prior to the nuclease agent or the one or more nucleic acids encoding the nuclease agent. In some such methods, the nucleic acid construct is administered after the nuclease agent or the one or more nucleic acids encoding the nuclease agent. In some such methods, the subject is a human subject. In some such methods, the subject is a neonatal subject. In some such methods, the neonatal subject is a human subject within 24 weeks after birth. In some such methods, the neonatal subject is a human subject within 12 weeks after birth. In some such methods, the neonatal subject is a human subject within 8 weeks after birth. In some such methods, the neonatal subject is a human subject within 4 weeks after birth. In some such methods, the subject is not a neonatal subject.

In another aspect, provided are compositions comprising a nucleic acid construct comprising a coding sequence for lysosomal alpha-glucosidase, wherein the lysosomal alpha-glucosidase coding sequence is CpG-depleted relative to a wild type lysosomal alpha-glucosidase coding sequence. In some such compositions, the lysosomal alpha-glucosidase lacks the lysosomal alpha-glucosidase signal peptide and propeptide. In some such compositions, the lysosomal alpha-glucosidase comprises the sequence set forth in SEQ ID NO: 173. In some such compositions, the lysosomal alpha-glucosidase consists of the sequence set forth in SEQ ID NO: 173. In some such compositions, the lysosomal alpha-glucosidase coding sequence is codon-optimized and CpG-depleted. In some such compositions, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and 205-212, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176. In some such compositions, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and 205-212 and encodes a lysosomal alpha-glucosidase protein comprising SEQ ID NO: 173, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176 and encodes a lysosomal alpha-glucosidase protein comprising SEQ ID NO: 173. In some such compositions, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and 205-212, is codon-optimized and CpG-depleted, and encodes a lysosomal alpha-glucosidase protein comprising SEQ ID NO: 173, optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176, is codon-optimized and CpG-depleted, and encodes a lysosomal alpha-glucosidase protein comprising SEQ ID NO: 173. In some such compositions, the lysosomal alpha-glucosidase coding sequence comprises the sequence set forth in any one of SEQ ID NOS: 174-182 and 205-212, optionally wherein the lysosomal alpha-glucosidase coding sequence comprises the sequence set forth in SEQ ID NO: 176. In some such compositions, the lysosomal alpha-glucosidase coding sequence consists of the sequence set forth in any one of SEQ ID NOS: 174-182 and 205-212, optionally wherein the lysosomal alpha-glucosidase coding sequence consists of the sequence set forth in SEQ ID NO: 176.

In some such compositions, the nucleic acid construct comprises a splice acceptor upstream of the lysosomal alpha-glucosidase coding sequence. In some such compositions, the nucleic acid construct comprises a polyadenylation signal or sequence downstream of the lysosomal alpha-glucosidase coding sequence. In some such compositions, the nucleic acid construct comprises a splice acceptor upstream of the lysosomal alpha-glucosidase coding sequence, and the nucleic acid construct comprises a polyadenylation signal or sequence downstream of the lysosomal alpha-glucosidase coding sequence. In some such compositions, the nucleic acid construct does not comprise a homology arm. In some such compositions, the nucleic acid construct comprises homology arms. In some such compositions, the nucleic acid construct does not comprise a promoter that drives the expression of the lysosomal alpha-glucosidase. In some such compositions, the lysosomal alpha-glucosidase coding sequence is operably linked to a promoter, optionally wherein the promoter is a liver-specific promoter. In some such compositions, the nucleic acid construct is single-stranded DNA or double-stranded DNA. In some such compositions, the nucleic acid construct is single-stranded DNA.

In some such compositions, the nucleic acid construct comprises from 5′ to 3′: a splice acceptor, the lysosomal alpha-glucosidase coding sequence, and a polyadenylation signal or sequence, wherein the lysosomal alpha-glucosidase coding sequence comprises any one of SEQ ID NOS: 174-182 and 205-212, optionally wherein the lysosomal alpha-glucosidase coding sequence comprises SEQ ID NO: 176, wherein the nucleic acid construct does not comprise a promoter that drives the expression of the lysosomal alpha-glucosidase, and wherein the nucleic acid construct does not comprise a homology arm.

In some such compositions, the nucleic acid construct is in a nucleic acid vector or a lipid nanoparticle. In some such compositions, the nucleic acid construct is in the nucleic acid vector. In some such compositions, the nucleic acid vector is a viral vector. In some such compositions, the nucleic acid vector is an adeno-associated viral (AAV) vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160. In some such compositions, the AAV vector is a single-stranded AAV (ssAAV) vector. In some such compositions, the AAV vector is derived from an AAV8 vector, an AAV3B vector, an AAV5 vector, an AAV6 vector, an AAV7 vector, an AAV9 vector, an AAVrh.74 vector, or an AAVhu.37 vector. In some such compositions, the AAV vector is a recombinant AAV8 (rAAV8) vector. In some such compositions, the AAV vector is a single-stranded rAAV8 vector.

In some such compositions, the nucleic acid construct comprises from 5′ to 3′: a splice acceptor, the lysosomal alpha-glucosidase coding sequence, and a polyadenylation signal or sequence, wherein the lysosomal alpha-glucosidase coding sequence comprises any one of SEQ ID NOS: 174-182 and 205-212, optionally wherein the lysosomal alpha-glucosidase coding sequence comprises SEQ ID NO: 176, wherein the nucleic acid construct does not comprise a promoter that drives the expression of the lysosomal alpha-glucosidase, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160. In some such compositions, the nucleic acid construct is CpG-depleted.

Some such compositions further comprise a nuclease agent that targets a nuclease target site in a target genomic locus. In some such compositions, the target genomic locus is an albumin gene, optionally wherein the albumin gene is a human albumin gene. In some such compositions, the nuclease target site is in intron 1 of the albumin gene. In some such compositions, the nuclease agent comprises: (a) a zinc finger nuclease (ZFN); (b) a transcription activator-like effector nuclease (TALEN); or (c) (i) a Cas protein or a nucleic acid encoding the Cas protein; and (ii) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

In some such compositions, the nuclease agent comprises: (a) a Cas protein or a nucleic acid encoding the Cas protein; and (b) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence. In some such compositions, the guide RNA target sequence is in intron 1 of an albumin gene. In some such compositions, the albumin gene is a human albumin gene. In some such compositions: (I) the DNA-targeting segment comprises at least 17, at least 18, at least 19, or at least 20 contiguous nucleotides of the sequence set forth in any one of SEQ ID NOS: 30-61, optionally wherein the DNA-targeting segment comprises at least 17, at least 18, at least 19, or at least 20 contiguous nucleotides of the sequence set forth in any one of SEQ ID NOS: 36, 30, 33, and 41; and/or (II) the DNA-targeting segment is at least 90% or at least 95% identical to the sequence set forth in any one of SEQ ID NOS: 30-61, optionally wherein the DNA-targeting segment is at least 90% or at least 95% identical to the sequence set forth in any one of SEQ ID NOS: 36, 30, 33, and 41. In some such compositions, the DNA-targeting segment comprises any one of SEQ ID NOS: 30-61, optionally wherein the DNA-targeting segment comprises any one of SEQ ID NOS: 36, 30, 33, and 41. In some such compositions, the DNA-targeting segment consists of any one of SEQ ID NOS: 30-61, optionally wherein the DNA-targeting segment consists of any one of SEQ ID NOS: 36, 30, 33, and 41. In some such compositions, the guide RNA comprises any one of SEQ ID NOS: 62-125, optionally wherein the guide RNA comprises any one of SEQ ID NOS: 68, 100, 62, 94, 65, 97, 73, and 105. In some such compositions: (I) the DNA-targeting segment comprises at least 17, at least 18, at least 19, or at least 20 contiguous nucleotides of SEQ ID NO: 36; and/or (II) the DNA-targeting segment is at least 90% or at least 95% identical to SEQ ID NO: 36. In some such compositions, the DNA-targeting segment comprises SEQ ID NO: 36. In some such compositions, the DNA-targeting segment consists of SEQ ID NO: 36. In some such compositions, the guide RNA comprises SEQ ID NO: 68 or 100.

In some such compositions, the guide RNA in the form of RNA. In some such compositions, the guide RNA comprises at least one modification. In some such compositions, the at least one modification comprises a 2′-O-methyl-modified nucleotide. In some such compositions, the at least one modification comprises a phosphorothioate bond between nucleotides. In some such compositions, the at least one modification comprises a modification at one or more of the first five nucleotides at the 5′ end of the guide RNA. In some such compositions, the at least one modification comprises a modification at one or more of the last five nucleotides at the 3′ end of the guide RNA. In some such compositions, the at least one modification comprises phosphorothioate bonds between the first four nucleotides at the 5′ end of the guide RNA. In some such compositions, the at least one modification comprises phosphorothioate bonds between the last four nucleotides at the 3′ end of the guide RNA. In some such compositions, the at least one modification comprises 2′-O-methyl-modified nucleotides at the first three nucleotides at the 5′ end of the guide RNA. In some such compositions, the at least one modification comprises 2′-O-methyl-modified nucleotides at the last three nucleotides at the 3′ end of the guide RNA. In some such compositions, the at least one modification comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA. In some such compositions, the guide RNA is a single guide RNA (sgRNA). In some such compositions, the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

In some such compositions, the Cas protein is a Cas9 protein. In some such compositions, the Cas9 protein is derived from a Streptococcus pyogenes Cas9 protein, a Staphylococcus aureus Cas9 protein, a Campylobacter jejuni Cas9 protein, a Streptococcus thermophilus Cas9 protein, or a Neisseria meningitidis Cas9 protein. In some such compositions, the Cas protein is derived from a Streptococcus pyogenes Cas9 protein. In some such compositions, the Cas protein comprises the sequence set forth in SEQ ID NO: 11. In some such compositions, the nucleic acid encoding the Cas protein is codon-optimized for expression in a mammalian cell or a human cell. In some such compositions, the composition comprises the nucleic acid encoding the Cas protein, wherein the nucleic acid comprises an mRNA encoding the Cas protein. In some such compositions, the mRNA encoding the Cas protein comprises at least one modification. In some such compositions, the mRNA encoding the Cas protein is modified to comprise a modified uridine at one or more or all uridine positions. In some such compositions, the modified uridine is pseudouridine or N1-methyl-pseudouridine, optionally N1-methyl-pseudouridine. In some such compositions, the mRNA encoding the Cas protein is fully substituted with pseudouridine or N1-methyl-pseudouridine, optionally N1-methyl-pseudouridine. In some such compositions, the mRNA encoding the Cas protein comprises a 5′ cap. In some such compositions, the mRNA encoding the Cas protein comprises a polyadenylation sequence. In some such compositions, the mRNA encoding the Cas protein comprises the sequence set forth in SEQ ID NO: 226, 225, or 12.

In some such compositions, the composition comprises the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine or N1-methyl-pseudouridine, optionally N1-methyl-pseudouridine, comprises a 5′ cap, and comprises a polyadenylation sequence. In some such compositions, the guide RNA in the form of RNA, and the guide RNA comprises SEQ ID NO: 68 or 100, and wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 226, 225, or 12. In some such compositions, the nucleic acid construct comprises from 5′ to 3′: a splice acceptor, the lysosomal alpha-glucosidase coding sequence, and a polyadenylation signal or sequence, wherein the lysosomal alpha-glucosidase coding sequence comprises any one of SEQ ID NOS: 174-182 and 205-212, optionally wherein the lysosomal alpha-glucosidase coding sequence comprises SEQ ID NO: 176, wherein the nucleic acid construct does not comprise a promoter that drives the expression of the lysosomal alpha-glucosidase, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

In some such compositions, the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine or N1-methyl-pseudouridine, optionally N1-methyl-pseudouridine, comprises a 5′ cap, and comprises a polyadenylation sequence. In some such compositions, the nucleic acid construct comprises from 5′ to 3′: a splice acceptor, the lysosomal alpha-glucosidase coding sequence, and a polyadenylation signal or sequence, wherein the lysosomal alpha-glucosidase coding sequence comprises any one of SEQ ID NOS: 174-182 and 205-212, optionally wherein the lysosomal alpha-glucosidase coding sequence comprises SEQ ID NO: 176, wherein the nucleic acid construct does not comprise a promoter that drives the expression of the lysosomal alpha-glucosidase, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

In some such compositions, the Cas protein or the nucleic acid encoding the Cas protein and the guide RNA or the one or more DNAs encoding the guide RNA are associated with a lipid nanoparticle. In some such compositions, the lipid nanoparticle comprises a cationic lipid, a neutral lipid, a helper lipid, and a stealth lipid. In some such compositions, the cationic lipid is Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate). In some such compositions, the neutral lipid is distearoylphosphatidylcholine or 1,2-distearoyl-sn-glycero-3-phosphocholine (DSPC). In some such compositions, the helper lipid is cholesterol. In some such compositions, the stealth lipid is 1,2-dimyristoyl-rac-glycero-3-methoxypolyethylene glycol-2000 (PEG2k-DMG). In some such compositions, the cationic lipid is Lipid A, the neutral lipid is DSPC, the helper lipid is cholesterol, and the stealth lipid is PEG2k-DMG. In some such compositions, the lipid nanoparticle comprises four lipids at the following molar ratios: about 50 mol% Lipid A, about 9 mol% DSPC, about 38 mol% cholesterol, and about 3 mol% PEG2k-DMG.

In some such compositions, the albumin gene is a human albumin gene, wherein the guide RNA in the form of RNA, and the guide RNA comprises SEQ ID NO: 68 or 100, wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 226, 225, or 12, and wherein the guide RNA and the mRNA encoding the Cas protein are associated with a lipid nanoparticle comprising Lipid A, DSPC, cholesterol, and PEG2k-DMG, optionally at the following molar ratios: about 50 mol% Lipid A, about 9 mol% DSPC, about 38 mol% cholesterol, and about 3 mol% PEG2k-DMG. In some such compositions, the nucleic acid construct comprises from 5′ to 3′: a splice acceptor, the lysosomal alpha-glucosidase coding sequence, and a polyadenylation signal or sequence, wherein the lysosomal alpha-glucosidase coding sequence comprises any one of SEQ ID NOS: 174-182 and 205-212, optionally wherein the lysosomal alpha-glucosidase coding sequence comprises SEQ ID NO: 176, wherein the nucleic acid construct does not comprise a promoter that drives the expression of the lysosomal alpha-glucosidase, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

In some such compositions, the albumin gene is a human albumin gene, wherein the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine or N1-methyl-pseudouridine, optionally N1-methyl-pseudouridine, comprises a 5′ cap, and comprises a polyadenylation sequence, and wherein the guide RNA and the mRNA encoding the Cas protein are associated with a lipid nanoparticle comprising Lipid A, DSPC, cholesterol, and PEG2k-DMG, optionally at the following molar ratios: about 50 mol% Lipid A, about 9 mol% DSPC, about 38 mol% cholesterol, and about 3 mol% PEG2k-DMG. In some such compositions, the nucleic acid construct comprises from 5′ to 3′: a splice acceptor, the lysosomal alpha-glucosidase coding sequence, and a polyadenylation signal or sequence, wherein the lysosomal alpha-glucosidase coding sequence comprises any one of SEQ ID NOS: 174-182 and 205-212, optionally wherein the lysosomal alpha-glucosidase coding sequence comprises SEQ ID NO: 176, wherein the nucleic acid construct does not comprise a promoter that drives the expression of the lysosomal alpha-glucosidase, wherein the nucleic acid construct does not comprise a homology arm, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

Some such compositions are for use in a method of inserting the lysosomal alpha-glucosidase coding sequence into a target genomic locus in a cell or a population of cells. Some such compositions are for use in a method of expressing the lysosomal alpha-glucosidase from a target genomic locus in a cell or a population of cells. Some such compositions are for use in a method of expressing the lysosomal alpha-glucosidase in a cell or a population of cells. Some such compositions are for use in a method of inserting the lysosomal alpha-glucosidase coding sequence into a target genomic locus in a cell or a population of cells in a subject. Some such compositions are for use in a method of expressing the lysosomal alpha-glucosidase from a target genomic locus in a cell or a population of cells in a subject. Some such compositions are for use in a method of expressing the lysosomal alpha-glucosidase in a cell or a population of cells in a subject. Some such compositions are for use in a method of treating a lysosomal alpha-glucosidase deficiency in a subject in need thereof. Some such compositions are for use in a method of reducing glycogen accumulation in a tissue in a subject in need thereof. Some such compositions are for use in a method of treating Pompe disease in a subject in need thereof. Some such compositions are for use in a method of preventing or reducing the onset of a sign or symptom of Pompe disease in a neonatal subject in need thereof.

In another aspect, provided are cells comprising any of the above compositions. In some such cells, the nucleic acid construct is integrated into a target genomic locus, and wherein the lysosomal alpha-glucosidase is expressed from the target genomic locus, or wherein the nucleic acid construct is integrated into intron 1 of an endogenous albumin locus, and wherein the lysosomal alpha-glucosidase is expressed from the endogenous albumin locus. In some such cells, the cell is a liver cell. In some such cells, the liver cell is a hepatocyte. In some such cells, the cell is a human cell. In some such cells, the cell is a neonatal cell. In some such cells, the neonatal cell is from a human neonatal subject within 24 weeks after birth. In some such cells, the neonatal cell is from a human neonatal subject within 12 weeks after birth. In some such cells, the neonatal cell is from a human neonatal subject within 8 weeks after birth. In some such cells, the neonatal cell is from a human neonatal subject within 4 weeks after birth. In some such cells, the cell is not a neonatal cell. In some such cells, the cell is in vivo. In some such cells, the cell is in vitro or ex vivo.

In another aspect, provided are methods of inserting a lysosomal alpha-glucosidase coding sequence into a target genomic locus in a cell or a population of cells. Some such methods comprise administering to the cell or the population of cells any of the above compositions, wherein the nuclease agent cleaves the nuclease target site in the target genomic locus, and the nucleic acid construct is inserted into the target genomic locus. In another aspect, provided are methods of expressing a lysosomal alpha-glucosidase in a cell or a population of cells. Some such methods comprise administering to the cell or the population of cells any of the above compositions, wherein the lysosomal alpha-glucosidase coding sequence is operably linked to a promoter in the nucleic acid construct and is expressed in the cell or population of cells. In another aspect, provided are methods of expressing a lysosomal alpha-glucosidase from a target genomic locus in a cell or a population of cells. Some such methods comprise administering to the cell or the population of cells any of the above compositions, wherein the nuclease agent cleaves the nuclease target site in the target genomic locus, the nucleic acid construct is inserted into the target genomic locus to create a modified target genomic locus, and the lysosomal alpha-glucosidase is expressed from the modified target genomic locus. In some such methods, the cell is a liver cell or the population of cells is a population of liver cells. In some such methods, the cell is a hepatocyte or the population of cells is a population of hepatocytes. In some such methods, the cell is a human cell or the population of cells is a population of human cells. In some such methods, the cell is a neonatal cell or the population of cells is a population of neonatal cells. In some such methods, the neonatal cell or the population of neonatal cells is from a human neonatal subject within 24 weeks after birth. In some such methods, the neonatal cell or the population of neonatal cells is from a human neonatal subject within 12 weeks after birth. In some such methods, the neonatal cell or the population of neonatal cells is from a human neonatal subject within 8 weeks after birth. In some such methods, the neonatal cell or the population of neonatal cells is from a human neonatal subject within 4 weeks after birth. In some such methods, the cell is not a neonatal cell or the population of cells is not a population of neonatal cells. In some such methods, the cell is in vitro or ex vivo or the population of cells is in vitro or ex vivo. In some such methods, the cell is in vivo in a subject or the population of cells is in vivo in a subject.

In another aspect, provided are methods of inserting a lysosomal alpha-glucosidase coding sequence into a target genomic locus in a cell in a subject. Some such methods comprise administering to the subject any of the above compositions, wherein the nuclease agent cleaves the nuclease target site in the target genomic locus, and the nucleic acid construct is inserted into the target genomic locus. In another aspect, provided are methods of expressing a lysosomal alpha-glucosidase in a cell in a subject. Some such methods comprise administering to the subject any of the above compositions, wherein the lysosomal alpha-glucosidase coding sequence is operably linked to a promoter in the nucleic acid construct and is expressed in the cell. In another aspect, provided are methods of expressing a lysosomal alpha-glucosidase from a target genomic locus in a cell in a subject. Some such methods comprise administering to the subject any of the above compositions, wherein the nuclease agent cleaves the nuclease target site in the target genomic locus, the nucleic acid construct is inserted into the target genomic locus to create a modified target genomic locus, and the lysosomal alpha-glucosidase is expressed from the modified target genomic locus. In some such methods, the cell is a liver cell. In some such methods, the cell is a hepatocyte. In some such methods, the cell is a human cell. In some such methods, the cell is a neonatal cell. In some such methods, the neonatal subject is a human subject within 24 weeks after birth. In some such methods, the neonatal subject is a human subject within 12 weeks after birth. In some such methods, the neonatal subject is a human subject within 8 weeks after birth. In some such methods, the neonatal subject is a human subject within 4 weeks after birth. In some such methods, the cell is not a neonatal cell.

In another aspect, provided are methods of treating a lysosomal alpha-glucosidase deficiency in a subject in need thereof. Some such methods comprise administering to the subject any of the above compositions, wherein the lysosomal alpha-glucosidase coding sequence is operably linked to a promoter in the nucleic acid construct and is expressed in the subject. Some such methods comprise administering to the subject any of the above compositions, wherein the nuclease agent cleaves the nuclease target site in the target genomic locus, the nucleic acid construct is inserted into the target genomic locus to create a modified target genomic locus, and the lysosomal alpha-glucosidase is expressed from the modified target genomic locus. In another aspect, provided are methods of reducing glycogen accumulation in a tissue in a subject in need thereof. Some such methods comprise administering to the subject any of the above compositions, wherein the lysosomal alpha-glucosidase coding sequence is operably linked to a promoter in the nucleic acid construct and is expressed in the subject and reduces glycogen accumulation in the tissue. Some such methods comprise administering to the subject any of the above compositions, wherein the nuclease agent cleaves the nuclease target site in the target genomic locus, the nucleic acid construct is inserted into the target genomic locus to create a modified target genomic locus, and the lysosomal alpha-glucosidase is expressed from the modified target genomic locus and reduces glycogen accumulation in the tissue. In some such methods, the subject has Pompe disease. In another aspect, provided are methods of treating Pompe disease in a subject in need thereof. Some such methods comprise administering to the subject any of the above compositions, wherein the lysosomal alpha-glucosidase coding sequence is operably linked to a promoter in the nucleic acid construct and is expressed in the subject, thereby treating the Pompe disease. Some such methods comprise administering to the subject any of the above compositions, wherein the nuclease agent cleaves the nuclease target site in the target genomic locus, the nucleic acid construct is inserted into the target genomic locus to create a modified target genomic locus, and the lysosomal alpha-glucosidase is expressed from the modified target genomic locus, thereby treating the Pompe disease. In some such methods, the Pompe disease is infantile-onset Pompe disease. In some such methods, the Pompe disease is late-onset Pompe disease.

In some such methods, the subject is a human subject. In some such methods, the subject is a neonatal subject. In some such methods, the neonatal subject is a human subject within 24 weeks after birth. In some such methods, the neonatal subject is a human subject within 12 weeks after birth. In some such methods, the neonatal subject is a human subject within 8 weeks after birth. In some such methods, the neonatal subject is a human subject within 4 weeks after birth. In some such methods, the subject is not a neonatal subject.

In some such methods, the method results in a therapeutically effective level of circulating lysosomal alpha-glucosidase in the subject. In some such methods, the method reduces glycogen accumulation in skeletal muscle, heart tissue, or central nervous system tissue in the subject. In some such methods, the method reduces glycogen accumulation in skeletal muscle and heart tissue in the subject. In some such methods, the method results in reduced glycogen levels in skeletal muscle and heart tissue in the subject comparable to wild type levels at the same age. In some such methods, the method improves muscle strength in the subject or prevents loss of muscle strength in the subject compared to a control subject. In some such methods, the method results in the subject having muscle strength comparable to wild type levels at the same age.

In another aspect, provided are methods of preventing or reducing the onset of a sign or symptom of Pompe disease in a subject in need thereof. Some such methods comprise administering to the subject any of the above compositions, wherein the lysosomal alpha-glucosidase coding sequence is operably linked to a promoter in the nucleic acid construct and is expressed in the subject, thereby preventing or reducing the onset of a sign or symptom of the Pompe disease in the subject. Some such methods comprise administering to the subject any of the above compositions, wherein the nuclease agent cleaves the nuclease target site, the nucleic acid construct is inserted into the target genomic locus to create a modified target genomic locus, and the lysosomal alpha-glucosidase is expressed from the modified target genomic locus, thereby preventing or reducing the onset of a sign or symptom of the Pompe disease in the subject. In some such methods, the Pompe disease is infantile-onset Pompe disease. In some such methods, the Pompe disease is late-onset Pompe disease.

In some such methods, the method results in a therapeutically effective level of circulating lysosomal alpha-glucosidase in the subject. In some such methods, the method prevents or reduces glycogen accumulation in skeletal muscle, heart, or central nervous system tissue in the subject. In some such methods, the method prevents or reduces glycogen accumulation in skeletal muscle and heart tissue in the subject.

In some such methods, the subject is a human subject. In some such methods, the subject is a neonatal subject. In some such methods, the neonatal subject is a human subject within 24 weeks after birth. In some such methods, the neonatal subject is a human subject within 12 weeks after birth. In some such methods, the neonatal subject is a human subject within 8 weeks after birth. In some such methods, the neonatal subject is a human subject within 4 weeks after birth. In some such methods, the subject is not a neonatal subject.

In some such methods, the method results in increased expression of lysosomal alpha-glucosidase in the subject compared to a method comprising administering an episomal expression vector encoding the lysosomal alpha-glucosidase to a control subject. In some such methods, the method results in increased serum levels of the lysosomal alpha-glucosidase in the subject compared to a method comprising administering an episomal expression vector encoding the lysosomal alpha-glucosidase to a control subject. In some such methods, the method results in serum levels of the lysosomal alpha-glucosidase in the subject of at least about 1 µg/mL, at least about 2 µg/mL, at least about 3 µg/mL, at least about 4 µg/mL, at least about 5 µg/mL, at least about 6 µg/mL, at least about 7 µg/mL, at least about 8 µg/mL, at least about 9 µg/mL, or at least about 10 µg/mL. In some such methods, the method results in serum levels of the lysosomal alpha-glucosidase in the subject of at least about 2 µg/mL or at least about 5 µg/mL. In some such methods, the method results in serum levels of the lysosomal alpha-glucosidase in the subject of between about 2 µg/mL and about 30 µg/mL or between about 2 µg/mL and about 20 µg/mL. In some such methods, the method results in serum levels of the lysosomal alpha-glucosidase in the subject of between about 5 µg/mL and about 30 µg/mL or between about 5 µg/mL and about 20 µg/mL. In some such methods, the method achieves lysosomal alpha-glucosidase activity levels of at least about 40% of normal, at least about 45% of normal, at least about 50%, at least about 60%, at least about 70%, at least about 80%, at least about 90%, or 100% of normal. In some such methods: (I) the subject has infantile-onset Pompe disease, and the method achieves lysosomal alpha-glucosidase expression or activity levels of at least about 1% or more than about 1% of normal; or (II) the subject has late-onset Pompe disease, and the method achieves lysosomal alpha-glucosidase expression or activity levels of at least about 40% of normal or more than about 40% of normal. In some such methods, the expression or activity of the lysosomal alpha-glucosidase is at least 50% of the expression or activity of the lysosomal alpha-glucosidase at a peak level of expression measured for the subject at 24 weeks after the administering. In some such methods, the expression or activity of the lysosomal alpha-glucosidase is at least 50% of the expression or activity of the lysosomal alpha-glucosidase at a peak level of expression measured for the subject at one year after the administering. In some such methods, the expression or activity of the lysosomal alpha-glucosidase is at least 60% of the expression or activity of the lysosomal alpha-glucosidase at a peak level of expression measured for the subject at 24 weeks after the administering. In some such methods, the expression or activity of the lysosomal alpha-glucosidase is at least 50% of the expression or activity of the lysosomal alpha-glucosidase at a peak level of expression measured for the subject at two years after the administering. In some such methods, the expression or activity of the lysosomal alpha-glucosidase is at least 60% of the expression or activity of the lysosomal alpha-glucosidase at a peak level of expression measured for the subject at 2 years after the administering. In some such methods, the expression or activity of the lysosomal alpha-glucosidase is at least 60% of the expression or activity of the lysosomal alpha-glucosidase at a peak level of expression measured for the subject at 24 weeks after the administering.

In some such methods, the method further comprises assessing preexisting AAV immunity in the subject prior to administering the nucleic acid construct to the subject. In some such methods, the preexisting AAV immunity is preexisting AAV8 immunity. In some such methods, assessing preexisting AAV immunity comprises assessing immunogenicity using a total antibody immune assay or a neutralizing antibody assay.

In some such methods, the nucleic acid construct is administered simultaneously with the nuclease agent or the one or more nucleic acids encoding the nuclease agent. In some such methods, the nucleic acid construct is not administered simultaneously with the nuclease agent or the one or more nucleic acids encoding the nuclease agent. In some such methods, the nucleic acid construct is administered prior to the nuclease agent or the one or more nucleic acids encoding the nuclease agent. In some such methods, the nucleic acid construct is administered after the nuclease agent or the one or more nucleic acids encoding the nuclease agent.

### BRIEF DESCRIPTION OF THE FIGURES

FIG. 1 shows a schematic describing different human factor IX (hFIX) insertion templates tested in adult and neonatal mice.

FIG. 2A shows hFIX plasma levels in neonatal mice (n = 4-10 per group; male and female) and adult mice (n = 5 per group; female) at different time points post-administration of episomal hFIX (Episome), LNP-g666 + hFIX-HDR-500 template (HDR500), LNP-g666 + hFIX-HDR-800 template (HDR800), and LNP-g666 + hFIX-NHEJ template (NHEJ). The administration in neonatal mice occurred at P0 or P1. Saline-injected mice were used as negative controls. Data are shown on a log scale. FIG. 2B shows hFIX plasma levels in neonatal mice (n = 4-10 per group; male and female) at different time points post-administration of episomal hFIX (Episome), LNP-g666 + hFIX-HDR-500 template (HDR500), LNP-g666 + hFIX-HDR-800 template (HDR800), and LNP-g666 + hFIX-NHEJ template (NHEJ). The administration in neonatal mice occurred at P0 or P1. Saline-injected mice were used as negative controls. Data are shown on a linear scale.

FIG. 3 shows a schematic of LNP-g9860, which is a lipid nanoparticle containing Cas9 mRNA and sgRNA 9860 targeting human albumin (ALB) intron 1, and a recombinant AAV8 (rAAV8) capsid packaged with an anti-CD63:GAA insertion template.

FIG. 4 shows a schematic of targeting of GAA to the lysosome via fusion to anti-CD63 scFv.

FIG. 5 shows a schematic for CRISPR/Cas9-mediated insertion of an anti-CD63:GAA insertion template at the ALB locus. The human ALB locus is depicted, with the Cas9 cut site denoted with scissors. The splice acceptor site flanking the anti-CD63:GAA transgene in the insertion template is depicted. Following insertion and transcription driven by the endogenous ALB promoter, splicing between ALB exon 1 and the inserted anti-CD63:GAA DNA template occurs, diagrammed in dashed lines, to produce a hybrid ALB-anti-CD63:GAA mRNA. The ALB signal peptide promotes secretion of anti-CD63:GAA and is removed during protein maturation to yield anti-CD63:GAA in plasma.

FIG. 6 shows levels of anti-CD63:GAA in the serum over a 10-month time course following administration of LNP-g666 (1 mg/kg) and a recombinant AAV8 anti-CD63:GAA insertion template (1.2e13 vg/kg) (“Insertion”) or following administration of episomal AAV encoding of anti-CD63:GAA (4e12 vg/kg) (“Episomal”) to adult Pompe disease model male and female mice (n = 12; GAA ^{-/-}; CD63 ^{hu/hu}).

FIG. 7 shows glycogen levels in the heart, quadricep, diaphragm, and spinal cord in Pompe disease model mice (GAA ^{-/-}; CD63 ^{hu/hu}) at 10 months after administration of LNP-g666 and a recombinant AAV8 anti-CD63:GAA insertion template or at 10 months after administration of episomal AAV encoding anti-CD63:GAA to adult Pompe disease model male and female mice (n = 12; GAA ^{-/-}; CD63 ^{hu/hu).} Wild type GAA mice (GAA ^{+/+}; CD63 ^{hu/hu}; n = 4) and untreated Pompe disease model mice (n = 4) were used as controls. The horizontal dotted line is the lower limit of detection of the assay.

FIGS. 8A-8B show levels of anti-CD63:GAA in the serum over a 15-month time course following administration of LNP-g666 and a recombinant AAV8 anti-CD63:GAA insertion template (n = 10; male and female; “Insertion”) or following administration of episomal AAV encoding of anti-CD63:GAA (n = 6; male and female; “Episomal”) to neonatal (P1) Pompe disease model mice (GAA ^{-/-}; CD63 ^{hu/hu}). The horizontal dotted line is the lower limit of detection of the assay. The error bars in FIG. 8A are ± SD, and the error bars in FIG. 8B are ± SEM.

FIG. 9A shows glycogen levels in the heart, quadricep, gastrocnemius, and diaphragm in Pompe disease model mice (GAA ^{-/-}; CD63 ^{hu/hu}) at 3 months after administration of LNP-g666 and a recombinant AAV8 anti-CD63:GAA insertion template (n = 5; male and female, “I”) or at 3 months after administration of episomal AAV encoding anti-CD63:GAA (n = 3; male and female, “E”) to neonatal (P1) mice. Untreated Pompe disease model mice were used as controls.

FIG. 9B shows glycogen levels in the heart, quadricep, gastrocnemius, diaphragm, cerebrum, and spinal cord in Pompe disease model mice (GAA ^{-/-}; CD63 ^{hu/hu}) at 15 months after administration of LNP-g666 and a recombinant AAV8 anti-CD63:GAA insertion template (n = 10; male and female, “I”) or at 15 months after administration of episomal AAV encoding anti-CD63:GAA (n = 6; male and female, “E”) to neonatal (P1) mice. Untreated Pompe disease model mice (“U”) and wild type mice (“W”) were used as controls.

FIG. 10 shows grip strength in Pompe disease model mice (GAA ^{-/-}; CD63 ^{hu/hu}) at 15 months after administration of LNP-g666 and a recombinant AAV8 anti-CD63:GAA insertion template (n = 10; male and female, “P1 insertion AAV + LNP”) or at 15 months after administration of episomal AAV encoding anti-CD63:GAA (n = 6; male and female, “P1 episomal AAV”) to neonatal (P1) mice. Wild type GAA mice (GAA ^{+/+}; CD63 ^{hu/hu}; “Wild type”) and untreated Pompe disease model mice (“Untreated KO”) were used as controls.

FIG. 11 shows IFNα responses as measured by an IFNα ELISA in a primary human plasmacytoid DC-based assay. Various rAAV6 CpG-depleted anti-CD63:GAA templates were tested as compared to the first generation (non-CpG-depleted) anti-CD63:GAA template. rAAV6-GFP was used as a positive control, and a CpG-depleted (0 CpG) F9 template was used as a negative control.

FIG. 12 shows GAA enzymatic activity in the media after insertion of various anti-CD63:GAA and anti-TfR:GAA insertion templates into the albumin locus of primary human hepatocytes after delivery by rAAV2.

FIG. 13 shows GAA enzymatic activity in the media after insertion of various anti-CD63:GAA insertion templates into the albumin locus of primary human hepatocytes after delivery by rAAV6.

FIGS. 14A-14B show GAA serum expression in GAA ^{-/-} mice following administration of LNP-g666 and various recombinant AAV8 anti-CD63:GAA insertion templates. Untreated KO and untreated WT mice were used as controls.

FIG. 15 shows a schematic of LNP-g9860, which is a lipid nanoparticle containing Cas9 mRNA and sgRNA 9860 targeting human albumin (ALB) intron 1, and a recombinant AAV8 (rAAV8) capsid packaged with an anti-TfR:GAA insertion template.

FIG. 16 shows a schematic of targeting of GAA through multiple paths via fusion to anti-TfR scFv.

FIG. 17 shows a schematic for CRISPR/Cas9-mediated insertion of an anti-TfR:GAA insertion template at the ALB locus. The human ALB locus is depicted, with the Cas9 cut site denoted with scissors. The splice acceptor site flanking the anti-TfR:GAA transgene in the insertion template is depicted. Following insertion and transcription driven by the endogenous ALB promoter, splicing between ALB exon 1 and the inserted anti-TfR:GAA DNA template occurs, diagrammed in dashed lines, to produce a hybrid ALB-anti-TfR: GAA mRNA. The ALB signal peptide promotes secretion of anti-TfR:GAA and is removed during protein maturation to yield anti-TfR:GAA in plasma.

FIGS. 18A-18C show western blots showing that anti-human TfR antibody clones deliver GAA to the cerebrum of Tfrc^{hum} mice. Each lane = 1 mouse. Anti-mouse mTfR:GAA in Wt mice was used as a positive control. Anti-mouse mTfR:GAA in Tfrc^{hum} mice was used as a negative control.

FIG. 19 shows western blots showing that a subset of anti-hTfR antibody clones deliver mature GAA to the brain parenchyma in scfv:GAA format (delivery by HDD). Anti-mouse mTfR:GAA in Wt mice was used as a positive control. Anti-mouse mTfR:GAA in Tfrc^{hum} mice was used as a negative control.

FIG. 20 shows western blots showing that four selected anti-hTfR antibody clones deliver mature GAA to the brain parenchyma in scfv:GAA format (AAV8 episomal liver depot gene therapy). Anti-mouse mTfR:GAA in Wt mice was used as a positive control. Anti-mouse mTfR:GAA in Tftc^{hum} mice was used as a negative control.

FIG. 21 shows western blots showing that three selected episomal AAV8 liver depot anti-hTfR antibody clones deliver mature GAA to the CNS, heart, and muscle in Gaa^{-/-} /Tfrc^{hum} mice.

FIGS. 22A and 22B show that four selected episomal AAV8 liver depot anti-hTfR antibody clones rescue glycogen storage in CNS, heart, and muscle in Gaa^{-/-}/Tfrc^{hum} mice. Wt untreated mice were a positive control, and Gaa^{-/-} untreated mice were a negative control.

FIG. 22C shows that a selected episomal AAV8 liver depot anti-hTfR antibody clone rescues glycogen storage in dorsal root ganglia (DRGs) in Gaa^{-/-}/Tfrc^{hum} mice. Wt untreated mice were a positive control, and Gaa^{-/-} untreated mice were a negative control.

FIGS. 23A-23D show that three selected episomal AAV8 liver depot anti-hTfR antibody clones rescue glycogen storage in brain thalamus (FIG. 23A), brain cerebral cortex (FIG. 23B), brain hippocampus CA1 (FIG. 23C), and quadricep (FIG. 23D) in Gaa^{-/-} /Tfrc^{hum} mice. Wt untreated mice were a positive control, and Gaa^{-/-} untreated mice were a negative control.

FIG. 24A shows that insertion of anti-hTfR 12847scfv:GAA delivers mature GAA protein to CNS and muscle of Pompe model mice. FIG. 24B shows that insertion of anti-hTfR 12847scfv:GAA rescues glycogen storage in CNS and muscle of Pompe model mice. One Way ANOVA ^{∗}p<0.01; ^{∗∗}p<0.001; ^{∗∗∗}p<0.0001. Untreated Pompe disease model mice and wild type mice were used as controls. Mice injected with a recombinant AAV8 anti-TfR:GAA episomal template were used as a positive control. Mice injected with a recombinant AAV8 anti-TfR:GAA insertion template without LNP-g666 were used as a negative control.

FIGS. 25A and 25B show GAA enzymatic activity in the media after insertion of various anti-TfR:GAA insertion templates (CpG depleted and native) into the albumin locus of primary human hepatocytes after delivery by rAAV2.

FIG. 26A shows western blots showing that anti-human TfR antibody clones (0 CpG and native) deliver GAA to the brain (cerebrum) of 3-month-old Gaa^{-/-}/Tfrc^{hum} mice dosed intravenously with LNP-g666 (3 mg/kg) and various recombinant AAV8 anti-TfR:GAA or AAV8 anti-CD63:GAA insertion templates. Each lane = 1 mouse.

FIG. 26B shows that albumin insertion of anti-hTfR:GAA rescues glycogen storage in cerebrum, quadriceps, diaphragm, and heart in Gaa^{-/-}/Tfrc^{hum} mice dosed intravenously with LNP-g666 (3 mg/kg) and various recombinant AAV8 anti-TfR:GAA or AAV8 anti-CD63:GAA insertion templates. Glycogen levels were measured at 3 weeks post-administration. Wt untreated mice were a positive control, and Gaa^{-/-} untreated mice were a negative control.

FIG. 27A shows GAA activity in serum measured using a fluorometric substrate assay in cynomolgus macaques that were administered recombinant AAV8 containing a CpG depleted anti-CD63:GAA template and LNP-g9860. Three different AAV8 doses were used (0.3e13vg/kg, 1.5e13vg/kg, and 5.6e13vg/kg) with a 3 mg/kg LNP dose. N=1 in the vehicle control group, and N=3 in the dosed groups.

FIG. 27B shows expression of mature GAA in tissue lysates from cynomolgus macaques that were administered recombinant AAV8 containing a CpG depleted anti-CD63:GAA template and LNP-g9860. Three different AAV8 doses were used (0.3e13vg/kg, 1.5e13vg/kg, and 5.6e13vg/kg) with a 3 mg/kg LNP dose. N=1 in the vehicle control group, and N=3 in the dosed groups. Tissues were collected at sacrifice (Day 89) and probed by western blot for presence of a 76 kDa lysosomal form of GAA.

### DEFINITIONS

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

Proteins are said to have an “N-terminus” and a “C-terminus.” The term “N-terminus” relates to the start of a protein or polypeptide, terminated by an amino acid with a free amine group (—NH2). The term “C-terminus” relates to the end of an amino acid chain (protein or polypeptide), terminated by a free carboxyl group (—COOH).

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

The term “genomically integrated” refers to a nucleic acid that has been introduced into a cell such that the nucleotide sequence integrates into the genome of the cell. Any protocol may be used for the stable incorporation of a nucleic acid into the genome of a cell.

The term “viral vector” refers to a recombinant nucleic acid that includes at least one element of viral origin and includes elements sufficient for or permissive of packaging into a viral vector particle. The vector and/or particle can be utilized for the purpose of transferring DNA, RNA, or other nucleic acids into cells in vitro, ex vivo, or in vivo. Numerous forms of viral vectors are known.

The term “isolated” with respect to cells, tissues (e.g., liver samples), proteins, and nucleic acids includes cells, tissues (e.g., liver samples), proteins, and nucleic acids that are relatively purified with respect to other bacterial, viral, cellular, or other components that may normally be present in situ, up to and including a substantially pure preparation of the cells, tissues (e.g., liver samples), proteins, and nucleic acids. The term “isolated” also includes cells, tissues (e.g., liver samples), proteins, and nucleic acids that have no naturally occurring counterpart, have been chemically synthesized and are thus substantially uncontaminated by other cells, tissues (e.g., liver samples), proteins, and nucleic acids, or has been separated or purified from most other components (e.g., cellular components) with which they are naturally accompanied (e.g., other cellular proteins, polynucleotides, or cellular components).

The term “wild type” includes entities having a structure and/or activity as found in a normal (as contrasted with mutant, diseased, altered, or so forth) state or context. Wild type genes and polypeptides often exist in multiple different forms (e.g., alleles).

The term “endogenous sequence” refers to a nucleic acid sequence that occurs naturally within a cell or animal. For example, an endogenous ALB sequence of a human refers to a native ALB sequence that naturally occurs at the ALB locus in the human.

“Exogenous” molecules or sequences include molecules or sequences that are not normally present in a cell in that form. Normal presence includes presence with respect to the particular developmental stage and environmental conditions of the cell. An exogenous molecule or sequence, for example, can include a mutated version of a corresponding endogenous sequence within the cell, such as a humanized version of the endogenous sequence, or can include a sequence corresponding to an endogenous sequence within the cell but in a different form (i.e., not within a chromosome). In contrast, endogenous molecules or sequences include molecules or sequences that are normally present in that form in a particular cell at a particular developmental stage under particular environmental conditions.

The term “heterologous” when used in the context of a nucleic acid or a protein indicates that the nucleic acid or protein comprises at least two segments that do not naturally occur together in the same molecule. For example, the term “heterologous,” when used with reference to segments of a nucleic acid or segments of a protein, indicates that the nucleic acid or protein comprises two or more sub-sequences that are not found in the same relationship to each other (e.g., joined together) in nature. As one example, a “heterologous” region of a nucleic acid vector is a segment of nucleic acid within or attached to another nucleic acid molecule that is not found in association with the other molecule in nature. For example, a heterologous region of a nucleic acid vector could include a coding sequence flanked by sequences not found in association with the coding sequence in nature. Likewise, a “heterologous” region of a protein is a segment of amino acids within or attached to another peptide molecule that is not found in association with the other peptide molecule in nature (e.g., a fusion protein, or a protein with a tag). Similarly, a nucleic acid or protein can comprise a heterologous label or a heterologous secretion or localization sequence.

“Codon optimization” (i.e., “codon optimized” sequences) takes advantage of the degeneracy of codons, as exhibited by the multiplicity of three-base pair codon combinations that specify an amino acid, and generally includes a process of modifying a nucleic acid sequence for enhanced expression in particular host cells by replacing at least one codon of the native sequence with a codon that is more frequently or most frequently used in the genes of the host cell while maintaining the native amino acid sequence. For example, a nucleic acid encoding a polypeptide of interest can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host cell, as compared to the naturally occurring nucleic acid sequence. Codon usage tables are readily available, for example, at the “Codon Usage Database.” These tables can be adapted in a number of ways. See Nakamura et al. (2000) Nucleic Acids Res. 28(1):292, herein incorporated by reference in its entirety for all purposes. Computer algorithms for codon optimization of a particular sequence for expression in a particular host are also available (see, e.g., Gene Forge).

The term “locus” refers to a specific location of a gene (or significant sequence), DNA sequence, polypeptide-encoding sequence, or position on a chromosome of the genome of an organism. For example, an “ALB locus” may refer to the specific location of an ALB gene, ALB DNA sequence, albumin-encoding sequence, or ALB position on a chromosome of the genome of an organism that has been identified as to where such a sequence resides. An “ALB locus” may comprise a regulatory element of an ALB gene, including, for example, an enhancer, a promoter, 5′ and/or 3′ untranslated region (UTR), or a combination thereof.

The term “gene” refers to DNA sequences in a chromosome that may contain, if naturally present, at least one coding and at least one non-coding region. The DNA sequence in a chromosome that codes for a product (e.g., but not limited to, an RNA product and/or a polypeptide product) can include the coding region interrupted with non-coding introns and sequence located adjacent to the coding region on both the 5′ and 3′ ends such that the gene corresponds to the full-length mRNA (including the 5′ and 3′ untranslated sequences). Additionally, other non-coding sequences including regulatory sequences (e.g., but not limited to, promoters, enhancers, and transcription factor binding sites), polyadenylation signals, internal ribosome entry sites, silencers, insulating sequence, and matrix attachment regions may be present in a gene. These sequences may be close to the coding region of the gene (e.g., but not limited to, within 10 kb) or at distant sites, and they influence the level or rate of transcription and translation of the gene.

The term “allele” refers to a variant form of a gene. Some genes have a variety of different forms, which are located at the same position, or genetic locus, on a chromosome. A diploid organism has two alleles at each genetic locus. Each pair of alleles represents the genotype of a specific genetic locus. Genotypes are described as homozygous if there are two identical alleles at a particular locus and as heterozygous if the two alleles differ.

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a mouse cell, a rat cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety for all purposes.

“Operable linkage” or being “operably linked” includes juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

The methods and compositions provided herein employ a variety of different components. Some components throughout the description can have active variants and fragments. The term “functional” refers to the innate ability of a protein or nucleic acid (or a fragment or variant thereof) to exhibit a biological activity or function. The biological functions of functional fragments or variants may be the same or may in fact be changed (e.g., with respect to their specificity or selectivity or efficacy) in comparison to the original molecule, but with retention of the molecule’s basic biological function.

The term “variant” refers to a nucleotide sequence differing from the sequence most prevalent in a population (e.g., by one nucleotide) or a protein sequence different from the sequence most prevalent in a population (e.g., by one amino acid).

The term “fragment,” when referring to a protein, means a protein that is shorter or has fewer amino acids than the full-length protein. The term “fragment,” when referring to a nucleic acid, means a nucleic acid that is shorter or has fewer nucleotides than the full-length nucleic acid. A fragment can be, for example, when referring to a protein fragment, an N-terminal fragment (i.e., removal of a portion of the C-terminal end of the protein), a C-terminal fragment (i.e., removal of a portion of the N-terminal end of the protein), or an internal fragment (i.e., removal of a portion of each of the N-terminal and C-terminal ends of the protein). A fragment can be, for example, when referring to a nucleic acid fragment, a 5′ fragment (i.e., removal of a portion of the 3′ end of the nucleic acid), a 3′ fragment (i.e., removal of a portion of the 5′ end of the nucleic acid), or an internal fragment (i.e., removal of a portion each of the 5′ and 3′ ends of the nucleic acid).

“Sequence identity” or “identity” in the context of two polynucleotides or polypeptide sequences refers to the residues in the two sequences that are the same when aligned for maximum correspondence over a specified comparison window. When percentage of sequence identity is used in reference to proteins, residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values include the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized below.

TABLE 1

Amino Acid Categorizations

| | | | | | |
|---|---|---|---|---|---|
| Alanine | Ala | A | Nonpolar | Neutral | 1.8 |
| Arginine | Arg | R | Polar | Positive | -4.5 |
| Asparagine | Asn | N | Polar | Neutral | -3.5 |
| Aspartic acid | Asp | D | Polar | Negative | -3.5 |
| Cysteine | Cys | C | Nonpolar | Neutral | 2.5 |
| Glutamic acid | Glu | E | Polar | Negative | -3.5 |
| Glutamine | Gln | Q | Polar | Neutral | -3.5 |
| Glycine | Gly | G | Nonpolar | Neutral | -0.4 |
| Histidine | His | H | Polar | Positive | -3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | -3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | -1.6 |
| Serine | Ser | S | Polar | Neutral | -0.8 |
| Threonine | Thr | T | Polar | Neutral | -0.7 |
| Tryptophan | Trp | W | Nonpolar | Neutral | -0.9 |
| Tyrosine | Tyr | Y | Polar | Neutral | -1.3 |
| Valine | Val | V | Nonpolar | Neutral | 4.2 |

A “homologous” sequence (e.g., nucleic acid sequence) includes a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence. Homologous sequences can include, for example, orthologous sequence and paralogous sequences. Homologous genes, for example, typically descend from a common ancestral DNA sequence, either through a speciation event (orthologous genes) or a genetic duplication event (paralogous genes). “Orthologous” genes include genes in different species that evolved from a common ancestral gene by speciation. Orthologs typically retain the same function in the course of evolution. “Paralogous” genes include genes related by duplication within a genome. Paralogs can evolve new functions in the course of evolution.

The term “in vitro” includes artificial environments and to processes or reactions that occur within an artificial environment (e.g., a test tube or an isolated cell or cell line). The term “in vivo” includes natural environments (e.g., a cell or organism or body) and to processes or reactions that occur within a natural environment. The term “ex vivo” includes cells that have been removed from the body of an individual and processes or reactions that occur within such cells.

The term “antibody,” as used herein, includes immunoglobulin molecules comprising four polypeptide chains, two heavy (H) chains and two light (L) chains inter-connected by disulfide bonds. Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3. The term “high affinity” antibody refers to those antibodies having a binding affinity to their target of at least 10⁻⁹ M, at least 10⁻¹⁰ M; at least 10⁻¹¹ M; or at least 10⁻¹² M, as measured by surface plasmon resonance, e.g., BIACORE™ or solution-affinity ELISA. The term “antibody” may encompass any type of antibody, such as e.g. monoclonal or polyclonal. Moreover, the antibody may be or any origin, such as e.g. mammalian or non-mammalian. In one embodiment, the antibody may be mammalian or avian. In a further embodiment, the antibody may be or human origin and may further be a human monoclonal antibody.

The phrase “bispecific antibody” includes an antibody capable of selectively binding two or more epitopes. Bispecific antibodies generally comprise two different heavy chains, with each heavy chain specifically binding a different epitope-either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions, and such sequences can be expressed in a cell that expresses an immunoglobulin light chain. A typical bispecific antibody has two heavy chains each having three heavy chain CDRs, followed by (N-terminal to C-terminal) a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding or one or both of the heavy chains to one or both epitopes.

The phrase “heavy chain,” or “immunoglobulin heavy chain” includes an immunoglobulin heavy chain constant region sequence from any organism, and unless otherwise specified includes a heavy chain variable domain. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a CH1 domain, a hinge, a CH2 domain, and a CH3 domain. A functional fragment of a heavy chain includes a fragment that is capable of specifically recognizing an antigen (e.g., recognizing the antigen with a KD in the micromolar, nanomolar, or picomolar range), that is capable of expressing and secreting from a cell, and that comprises at least one CDR.

The phrase “light chain” includes an immunoglobulin light chain constant region sequence from any organism, and unless otherwise specified includes human kappa and lambda light chains. Light chain variable (VL) domains typically include three light chain CDRs and four framework (FR) regions, unless otherwise specified. Generally, a full-length light chain includes, from amino terminus to carboxyl terminus, a VL domain that includes FR1-CDR1-FR2-CDR2-FR3-CDR3-FR4, and a light chain constant domain. Light chains that can be used with this invention include, for example, those that do not selectively bind either the first or second antigen selectively bound by the antigen-binding protein. Suitable light chains include those that can be identified by screening for the most commonly employed light chains in existing antibody libraries (wet libraries or in silico), where the light chains do not substantially interfere with the affinity and/or selectivity of the antigen-binding domains of the antigen-binding proteins. Suitable light chains include those that can bind one or both epitopes that are bound by the antigen-binding regions of the antigen-binding protein.

The phrase “variable domain” includes an amino acid sequence of an immunoglobulin light or heavy chain (modified as desired) that comprises the following amino acid regions, in sequence from N-terminal to C-terminal (unless otherwise indicated): FR1, CDR1, FR2, CDR2, FR3, CDR3, FR4. A “variable domain” includes an amino acid sequence capable of folding into a canonical domain (VH or VL) having a dual beta sheet structure wherein the beta sheets are connected by a disulfide bond between a residue of a first beta sheet and a second beta sheet.

The phrase “complementarity determining region,” or the term “CDR,” includes an amino acid sequence encoded by a nucleic acid sequence of an organism’s immunoglobulin genes that normally (i.e., in a wild type animal) appears between two framework regions in a variable region of a light or a heavy chain of an immunoglobulin molecule (e.g., an antibody or a T cell receptor). A CDR can be encoded by, for example, a germline sequence or a rearranged or unrearranged sequence, and, for example, by a naive or a mature B cell or a T cell. In some circumstances (e.g., for a CDR3), CDRs can be encoded by two or more sequences (e.g., germline sequences) that are not contiguous (e.g., in an unrearranged nucleic acid sequence) but are contiguous in a B cell nucleic acid sequence, for example, as the result of splicing or connecting the sequences (e.g., V-D-J recombination to form a heavy chain CDR3).

The term “antibody fragment” refers to one or more fragments of an antibody that retain the ability to specifically bind to an antigen. Examples of binding fragments encompassed within the term “antibody fragment” include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) Nature 241:544-546), which consists of a VH domain, (vi) an isolated CDR, and (vii) an scFv, which consists of the two domains of the Fv fragment, VL and VH, joined by a synthetic linker to form a single protein chain in which the VL and VH regions pair to form monovalent molecules. Other forms of single chain antibodies, such as diabodies are also encompassed under the term “antibody” (see e.g., Holliger et al. (1993) Proc. Natl. Acad. Sci. U.S.A. 90:6444-6448; Poljak et al. (1994) Structure 2:1121-1123).

The phrase “Fc-containing protein” includes antibodies, bispecific antibodies, immunoadhesins, and other binding proteins that comprise at least a functional portion of an immunoglobulin CH2 and CH3 region. A “functional portion” refers to a CH2 and CH3 region that can bind a Fc receptor (e.g., an FcyR; or an FcRn, i.e., a neonatal Fc receptor), and/or that can participate in the activation of complement. If the CH2 and CH3 region contains deletions, substitutions, and/or insertions or other modifications that render it unable to bind any Fc receptor and also unable to activate complement, the CH2 and CH3 region is not functional.

Fc-containing proteins can comprise modifications in immunoglobulin domains, including where the modifications affect one or more effector function of the binding protein (e.g., modifications that affect FcyR binding, FcRn binding and thus half-life, and/or CDC activity). Such modifications include, but are not limited to, the following modifications and combinations thereof, with reference to EU numbering of an immunoglobulin constant region: 238, 239, 248, 249, 250, 252, 254, 255, 256, 258, 265, 267, 268, 269, 270, 272, 276, 278, 280, 283, 285, 286, 289, 290, 292, 293, 294, 295, 296, 297, 298, 301, 303, 305, 307, 308, 309, 311, 312, 315, 318, 320, 322, 324, 326, 327, 328, 329, 330, 331, 332, 333, 334, 335, 337, 338, 339, 340, 342, 344, 356, 358, 359, 360, 361, 362, 373, 375, 376, 378, 380, 382, 383, 384, 386, 388, 389, 398, 414, 416, 419, 428, 430, 433, 434, 435, 437, 438, and 439.

For example, and not by way of limitation, the binding protein is an Fc-containing protein and exhibits enhanced serum half-life (as compared with the same Fc-containing protein without the recited modification(s)) and have a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at 428 and/or 433 (e.g., L/R/SI/P/Q or K) and/or 434 (e.g., H/F or Y); or a modification at 250 and/or 428; or a modification at 307 or 308 (e.g., 308F, V308F), and 434. In another example, the modification can comprise a 428L (e.g., M428L) and 434S (e.g., N434S) modification; a 428L, 2591 (e.g., V259I), and a 308F (e.g., V308F) modification; a 433 K (e.g., H433K) and a 434 (e.g., 434Y) modification; a 252, 254, and 256 (e.g., 252Y, 254T, and 256E) modification; a 250Q and 428L modification (e.g., T250Q and M428L); a 307 and/or 308 modification (e.g., 308F or 308P).

The term “antigen-binding protein,” as used herein, refers to a polypeptide or protein (one or more polypeptides complexed in a functional unit) that specifically recognizes an epitope on an antigen, such as a cell-specific antigen and/or a target antigen of the present invention. An antigen-binding protein may be multi-specific. The term “multi-specific” with reference to an antigen-binding protein means that the protein recognizes different epitopes, either on the same antigen or on different antigens. A multi-specific antigen-binding protein of the present invention can be a single multifunctional polypeptide, or it can be a multimeric complex of two or more polypeptides that are covalently or non-covalently associated with one another. The term “antigen-binding protein” includes antibodies or fragments thereof of the present invention that may be linked to or co-expressed with another functional molecule, for example, another peptide or protein. For example, an antibody or fragment thereof can be functionally linked (e.g., by chemical coupling, genetic fusion, non-covalent association or otherwise) to one or more other molecular entities, such as a protein or fragment thereof to produce a bispecific or a multi-specific antigen-binding molecule with a second binding specificity.

As used herein, the term “epitope” refers to the portion of the antigen which is recognized by the multi-specific antigen-binding polypeptide. A single antigen (such as an antigenic polypeptide) may have more than one epitope. Epitopes may be defined as structural or functional. Functional epitopes are generally a subset of structural epitopes and are defined as those residues that directly contribute to the affinity of the interaction between the antigen-binding polypeptide and the antigen. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics. Epitopes formed from contiguous amino acids are typically retained on exposure to denaturing solvents, whereas epitopes formed by tertiary folding are typically lost on treatment with denaturing solvents.

The term “domain” refers to any part of a protein or polypeptide having a particular function or structure. Preferably, domains of the present invention bind to cell-specific or target antigens. Cell-specific antigen- or target antigen-binding domains, and the like, as used herein, include any naturally occurring, enzymatically obtainable, synthetic, or genetically engineered polypeptide or glycoprotein that specifically binds an antigen.

The term “half-body” or “half-antibody”, which are used interchangeably, refers to half of an antibody, which essentially contains one heavy chain and one light chain. Antibody heavy chains can form dimers, thus the heavy chain of one half-body can associate with heavy chain associated with a different molecule (e.g., another half-body) or another Fc-containing polypeptide. Two slightly different Fc-domains may “heterodimerize” as in the formation of bispecific antibodies or other heterodimers, -trimers, -tetramers, and the like. See Vincent and Murini (2012) Biotechnol. J. 7(12):1444-1450; and Shimamoto et al. (2012) MAbs 4(5):586-91. In one embodiment, the half-body variable domain specifically recognizes the internalization effector and the half body Fc-domain dimerizes with an Fc-fusion protein that comprises a replacement enzyme (e.g., a peptibody).

The term “single-chain variable fragment” or “scFv” includes a single chain fusion polypeptide containing an immunoglobulin heavy chain variable region (VH) and an immunoglobulin light chain variable region (VL). In some embodiments, the VH and VL are connect by a linker sequence of 10 to 25 amino acids. ScFv polypeptides may also include other amino acid sequences, such as CL or CH1 regions. ScFv molecules can be manufactured by phage display or made by directly subcloning the heavy and light chains from a hybridoma or B-cell. See Ahmad et al. (2012) Clin. Dev. Immunol. 2012:980250, herein incorporated by reference in its entirety for all purposes.

As used herein, the term “neonatal” in the context of humans covers human subjects up to or under the age of 1 year (52 weeks), preferably up to or under the age of 24 weeks, more preferably up to or under the age of 12 weeks, more preferably up to or under the age of 8 weeks, and even more preferably up to or under the age of 4 weeks. In certain embodiments, a neonatal human subject is up to 4 weeks of age. In certain embodiments, a neonatal human subject is up to 8 weeks of age. In another embodiment, a neonatal human subject is within 3 weeks after birth. In another embodiment, a neonatal human subject is within 2 weeks after birth. In another embodiment, a neonatal human subject is within 1 week after birth. In another embodiment, a neonatal human subject is within 7 days after birth. In another embodiment, a neonatal human subject is within 6 days after birth. In another embodiment, a neonatal human subject is within 5 days after birth. In another embodiment, a neonatal human subject is within 4 days after birth. In another embodiment, a neonatal human subject is within 3 days after birth. In another embodiment, a neonatal human subject is within 2 days after birth. In another embodiment, a neonatal human subject is within 1 day after birth. The time windows disclosed above are for human subjects and are also meant to cover the corresponding developmental time windows for other animals. As used herein, a “neonatal cell” is a cell of a neonatal subject, and a population of neonatal cells is a population of cells of a neonatal subject.

As used herein, a “control” as in a control sample or a control subject is a comparator for a measurement, e.g., a diagnostic measurement of a sign or symptom of a disease. In certain embodiments, a control can be a subject sample from the same subject an earlier time point, e.g., before a treatment intervention. In certain embodiments, a control can be a measurement from a normal subject, i.e., a subject not having the disease of the treated subject, to provide a normal control, e.g., an enzyme concentration or activity in a subject sample. In certain embodiments, a normal control can be a population control, i.e., the average of subjects in the general population. In certain embodiments, a control can be an untreated subject with the same disease. In certain embodiments, a control can be a subject treated with a different therapy, e.g., the standard of care. In certain embodiments, a control can be a subject or a population of subjects from a natural history study of subjects with the disease of the subject being compared. In certain embodiments, the control is matched for certain factors to the subject being tested, e.g., age, gender. In certain embodiments, a control may be a control level for a particular lab, e.g., a clinical lab. Selection of an appropriate control is within the ability of those of skill in the art.

Compositions or methods “comprising” or “including” one or more recited elements may include other elements not specifically recited. For example, a composition that “comprises” or “includes” a protein may contain the protein alone or in combination with other ingredients. The transitional phrase “consisting essentially of′ means that the scope of a claim is to be interpreted to encompass the specified elements recited in the claim and those that do not materially affect the basic and novel characteristic(s) of the claimed invention. Thus, the term “consisting essentially of′ when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which the event or circumstance does not.

Designation of a range of values includes all integers within or defining the range, and all subranges defined by integers within the range. For example, 5-10 nucleotides is understood as 5, 6, 7, 8, 9, or 10 nucleotides, whereas 5-10% is understood to contain 5% and all possible values through 10%.

At least 17 nucleotides of a 20 nucleotide sequence is understood to include 17, 18, 19, or 20 nucleotides of the sequence provided, thereby providing a upper limit even if one is not specifically provided as it would be clearly understood. Similarly, up to 3 nucleotides would be understood to encompass 0, 1, 2, or 3 nucleotides, providing a lower limit even if one is not specifically provided. When “at least”, “up to”, or other similar language modifies a number, it can be understood to modify each number in the series.

As used herein, “no more than” or “less than” is understood as the value adjacent to the phrase and logical lower values or integers, as logical from context, to zero. For example, a duplex region of “no more than 2 nucleotide base pairs” has a 2, 1, or 0 nucleotide base pairs. When “no more than” or “less than” is present before a series of numbers or a range, it is understood that each of the numbers in the series or range is modified.

As used herein, “detecting an analyte” and the like is understood as performing an assay in which the analyte can be detected, if present, wherein the analyte is present in an amount above the level of detection of the assay.

As used herein, “loss of function” is understood as an activity not being present, e.g., an enzyme activity not being present, for any reason. In certain embodiments, the absence of activity may be due to the absence of a protein having a function, e.g., protein is not transcribed or translated, protein is translated but not stable or not transported appropriately, either intracellularly or systemically. In certain embodiments, the absence of activity may be due to the presence of a mutation, e.g., point mutation, truncation, abnormal splicing, such that a protein is present, but not functional. A loss of function can be a partial or complete loss of function. In certain embodiments, various degrees of loss of function may be known that result in various conditions, severity of disease, or age of onset. As used herein, a loss of function is preferably not a transient loss of function, e.g., due to a stress response or other response that results in a temporary loss of a functional protein. Therapeutic interventions to correct for a loss of function of a protein may include compensation for the loss of function with the protein that is deficient, or with proteins that compensate for the loss of function, but that have a different sequence or structure than the protein for which the function is lost. It is understood that a loss of function of one protein may be compensated for by providing or altering the activity of another protein in the same biological pathway. In certain embodiments, the protein to compensate for the loss of function includes one or more of a truncation, mutation, or non-native sequence to direct trafficking of the protein, either intracellularly or systemically, to overcome the loss of function of the protein. The therapeutic intervention may or may not correct the loss of function of the protein in all cell types or tissues. The therapeutic intervention may include expression of the protein to compensate for a loss of function at a site remote from where the protein lacking function is typically expressed, e.g., where the deficiency results in dysfunction of a cell or organ. The therapeutic intervention may include expression of the protein in the liver to compensate for a loss of function at a site remote from the liver. A number of genetic mutations have been linked with specific loss of function mutations, in both humans and other species.

As used herein, “enzyme deficiency” is understood as an insufficient level of an enzyme activity due to a loss of function of the protein. An enzyme deficiency can be partial or total, and may result in differences in time of onset or severity of signs or symptoms of the enzyme deficiency depending on the level and site of the loss of function. As used herein, enzyme deficiency is preferably not a transient enzyme deficiency due to stress or other factors. A number of genetic mutations have been linked with enzyme deficiencies, in both humans and other species. In certain embodiments, enzyme deficiencies result in inborn errors of metabolism. In certain embodiments, enzyme deficiencies result in lysosomal storage diseases. In certain embodiments, enzyme deficiencies result in galactosemia. In certain embodiments, enzyme deficiencies result in bleeding disorders.

As used herein, it is understood that when the maximum amount of a value is represented by 100% (e.g., 100% inhibition or 100% encapsulation) that the value is limited by the method of detection. For example, 100% inhibition is understood as inhibition to a level below the level of detection of the assay, and 100% encapsulation is understood as no material intended for encapsulation can be detected outside the vesicles.

Unless otherwise apparent from the context, the term “about” encompasses values ± 5% of a stated value. In certain embodiments, the term “about” is understood to encompass tolerated variation or error within the art, e.g., 2 standard deviations from the mean, or the sensitivity of the method used to take a measurement, or a percent of a value as tolerated in the art, e.g., with age. When “about” is present before the first value of a series, it can be understood to modify each value in the series.

The term “and/or” refers to and encompasses any and all possible combinations of one or more of the associated listed items, as well as the lack of combinations when interpreted in the alternative (“or”).

The term “or” refers to any one member of a particular list and also includes any combination of members of that list.

The singular forms of the articles “a,” “an,” and “the” include plural references unless the context clearly dictates otherwise. For example, the term “a protein” or “at least one protein” can include a plurality of proteins, including mixtures thereof.

Statistically significant means p ≤0.05.

In the event of a conflict between a sequence in the application and an indicated accession number or position in an accession number, the sequence in the application predominates.

### DETAILED DESCRIPTION

### I. Overview

Compositions and methods for inserting a nucleic acid encoding a polypeptide of interest into a target genomic locus in a neonatal cell, a population of neonatal cells, or a neonatal subject or for expressing a nucleic acid encoding a polypeptide of interest in a neonatal cell, a population of neonatal cells, or a neonatal subject are provided. Also provided are methods of treating an enzyme deficiency, methods of treating a lysosomal storage disease, and methods of preventing or reducing the onset of a sign or symptom of an enzyme deficiency or a lysosomal storage disease in a subject. Also provided are neonatal cells or populations of neonatal cells comprising a nucleic acid construct comprising a coding sequence for a polypeptide of interest inserted into a target genomic locus.

In a specific example, compositions and methods for inserting a nucleic acid encoding a multidomain therapeutic protein (e.g., GAA fusion protein) into a target genomic locus in a neonatal cell, a population of neonatal cells, or a neonatal subject or for expressing a nucleic acid encoding a multidomain therapeutic protein (e.g., GAA fusion protein) from a target genomic locus in a neonatal cell, a population of neonatal cells, or a neonatal subject are also provided. Compositions and methods for treating GAA deficiency, reducing glycogen accumulation in a tissue, treating Pompe disease, or preventing or reducing the onset of a sign or symptom of Pompe disease in a neonatal subject are provided. Also provided are neonatal cells or populations of neonatal cells comprising a nucleic acid construct comprising a coding sequence for a multidomain therapeutic protein (e.g., GAA fusion protein) inserted into a target genomic locus.

The neonatal gene insertion platform described herein has advantages in terms of expression levels, durability of expression, and level of functional rescue of enzyme deficiencies over existing episomal platforms in neonates.

Also provided herein are nucleic acid constructs and compositions (e.g., episomal expression vectors) for expression of a multidomain therapeutic protein (e.g., GAA fusion protein). Also provided herein are nucleic acid constructs and compositions that allow insertion of a multidomain therapeutic protein (e.g., GAA fusion protein) coding sequence into a target genomic locus such as an endogenous ALB locus and/or expression of the multidomain therapeutic protein (e.g., GAA fusion protein) coding sequence. The nucleic acid constructs and compositions can be used in methods of integrating or inserting a multidomain therapeutic protein (e.g., GAA fusion protein) nucleic acid into a target genomic locus in a cell or a population of cells or a subject, methods of expressing a multidomain therapeutic protein (e.g., GAA fusion protein) in a cell or a population of cells or a subject, methods of reducing glycogen accumulation in a cell or a population of cells or a subject, methods of treating Pompe disease or GAA deficiency in a subject, and method of preventing or reducing the onset of a sign or symptom of Pompe disease in a subject, including neonatal cells and subjects.

More specifically, described herein in some embodiments is a therapeutic product based on the CRISPR/Cas9 gene editing technology and optionally contained in a lipid nanoparticle (LNP) delivery system, associated with a multidomain therapeutic protein (e.g., GAA fusion protein) DNA gene insertion template optionally contained in a recombinant adeno-associated virus serotype 8 (rAAV8). The CRISPR/Cas9 component has been designed to target and cut the double stranded DNA at a target gene locus (e.g., a safe harbor locus such as an ALB gene locus in hepatocytes), allowing for the multidomain therapeutic protein (e.g., GAA fusion protein) DNA template to be inserted in the genome at the target genomic locus. Transgene insertion provides a functional multidomain therapeutic protein (e.g., GAA fusion protein) gene, encoding the missing or defective genomic GAA in Pompe disease patients.

Some of the multidomain therapeutic protein (e.g., GAA fusion protein) coding sequences in the constructs disclosed herein are optimized for expression as compared to native GAA coding sequence. For example, the coding sequences in the constructs disclosed herein may include one or more modifications such as codon optimization (e.g., to human codons), depletion of CpG dinucleotides, mutation of cryptic splice sites, or any combination thereof. Other multidomain therapeutic protein coding sequences in the constructs disclosed herein comprise native GAA coding sequences.

### II. Compositions for Inserting Nucleic Acid Constructs Encoding and for Expressing Polypeptides of Interest in Cells or Neonatal Cells

Provided herein are nucleic acid constructs and compositions that allow insertion of a coding sequence for a polypeptide of interest into a target genomic locus such as an endogenous albumin (ALB) locus and/or expression of the coding sequence for the polypeptide of interest. Also provided herein are nucleic acid constructs and compositions (e.g. episomal expression vectors) for expression of a coding sequence for a polypeptide of interest. The nucleic acid constructs and compositions can be used in methods for integration into a target genomic locus and/or expression in a cell or a subject. Also provided are nuclease agents (e.g., targeting an endogenous ALB locus) or nucleic acids encoding nuclease agents to facilitate integration of the nucleic acid constructs into a target genomic locus such as an endogenous ALB locus.

Also provided herein are nucleic acid constructs and compositions that allow insertion of a multidomain therapeutic protein (e.g., GAA fusion protein) coding sequence into a target genomic locus such as an endogenous albumin (ALB) locus and/or expression of the multidomain therapeutic protein (e.g., GAA fusion protein) coding sequence. Also provided herein are nucleic acid constructs and compositions (e.g., episomal expression vectors) for expression of a multidomain therapeutic protein (e.g., GAA fusion protein). The nucleic acid constructs and compositions can be used in methods of introducing a nucleic acid construct comprising a multidomain therapeutic protein coding sequence into a cell or a population of cells or a subject, methods of inserting or integrating a nucleic acid construct comprising a multidomain therapeutic protein coding sequence into a target genomic locus, methods of expressing a multidomain therapeutic protein in a cell or a population of cells or a subject, methods of reducing glycogen accumulation in a cell or a population of cells or a tissue in a subject, and methods of treating Pompe disease or GAA deficiency in a subject. Also provided are nuclease agents (e.g., targeting an endogenous ALB locus) or nucleic acids encoding nuclease agents to facilitate integration of the nucleic acid constructs into a target genomic locus such as an endogenous ALB locus.

### A. Nucleic Acid Constructs Encoding a Polypeptide of Interest

The compositions and methods described herein include the use of a nucleic acid construct that comprises a coding sequence for a polypeptide of interest (e.g., an exogenous polypeptide coding sequence). The compositions and methods described herein can also include the use of a nucleic acid construct that comprises a polypeptide of interest coding sequence or a reverse complement of the polypeptide of interest coding sequence (e.g., an exogenous polypeptide coding sequence or a reverse complement of the exogenous polypeptide coding sequence). Such nucleic acid constructs can be for expression of the polypeptide of interest in a cell. Such nucleic acid constructs can be for insertion into a target genomic locus or into a cleavage site created by a nuclease agent or CRISPR/Cas system as disclosed elsewhere herein. The term cleavage site includes a DNA sequence at which a nick or double-strand break is created by a nuclease agent (e.g., a Cas9 protein complexed with a guide RNA). In some embodiments, a double-stranded break is created by a Cas9 protein complexed with a guide RNA, e.g., a Spy Cas9 protein complexed with a Spy Cas9 guide RNA. In some cases, the polypeptide of interest is an exogenous polypeptide as defined herein.

In a specific example, the compositions and methods described herein include the use of a nucleic acid construct that comprises a multidomain therapeutic protein coding sequence (a multidomain therapeutic protein nucleic acid). Such nucleic acid constructs can be for expression of the multidomain therapeutic protein in a cell. Such nucleic acid constructs can be for insertion into a target genomic locus following cleavage at a cleavage site by a nuclease agent or CRISPR/Cas system as disclosed elsewhere herein or can be for expression of the multidomain therapeutic protein without insertion into a target genomic locus or a cleavage site (e.g., in an episome). The term cleavage site includes a DNA sequence at which a nick or double-strand break is created by a nuclease agent (e.g., a Cas9 protein complexed with a guide RNA). In certain embodiments, the cleavage site includes a DNA sequence at which a double-strand break is created by a Cas9 protein complexed with a guide RNA, e.g., a Spy Cas9 protein complexed with a Spy Cas9 guide RNA.

The length of the nucleic acid constructs disclosed herein can vary. The construct can be, for example, from about 1 kb to about 5 kb, such as from about 1 kb to about 4.5 kb or about 1 kb to about 4 kb. An exemplary nucleic acid construct is between about 1 kb to about 5 kb in length or between about 1 kb to about 4 kb in length. Alternatively, a nucleic acid construct can be between about 1 kb to about 1.5 kb, about 1.5 kb to about 2 kb, about 2 kb to about 2.5 kb, about 2.5 kb to about 3 kb, about 3 kb to about 3.5 kb, about 3.5 kb to about 4 kb, about 4 kb to about 4.5 kb, or about 4.5 kb to about 5 kb in length. Alternatively, a nucleic acid construct can be, for example, no more than 5 kb, no more than 4.5 kb, no more than 4 kb, no more than 3.5 kb, no more than 3 kb, or no more than 2.5 kb in length.

The constructs can comprise deoxyribonucleic acid (DNA) or ribonucleic acid (RNA), can be single-stranded, double-stranded, or partially single-stranded and partially double-stranded, and can be introduced into a host cell in linear or circular (e.g., minicircle) form. See, e.g., US 2010/0047805, US 2011/0281361, and US 2011/0207221, each of which is herein incorporated by reference in their entirety for all purposes. If introduced in linear form, the ends of the construct can be protected (e.g., from exonucleolytic degradation) by known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in their entirety for all purposes. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. A construct can be introduced into a cell as part of a vector molecule having additional sequences such as, for example, replication origins, promoters, and genes encoding antibiotic resistance. A construct may omit viral elements. Moreover, constructs can be introduced as a naked nucleic acid, can be introduced as a nucleic acid complexed with an agent such as a liposome or poloxamer, or can be delivered by viruses (e.g., adenovirus, adeno-associated virus (AAV), herpesvirus, retrovirus, or lentivirus).

The constructs disclosed herein can be modified on either or both ends to include one or more suitable structural features as needed and/or to confer one or more functional benefit. For example, structural modifications can vary depending on the method(s) used to deliver the constructs disclosed herein to a host cell (e.g., use of viral vector delivery or packaging into lipid nanoparticles for delivery). Such modifications include, for example, terminal structures such as inverted terminal repeats (ITR), hairpin, loops, and other structures such as toroids. For example, the constructs disclosed herein can comprise one, two, or three ITRs or can comprise no more than two ITRs. Various methods of structural modifications are known.

Some constructs may be inserted so that their expression is driven by the endogenous promoter at the insertion site (e.g., the endogenous ALB promoter when the construct is integrated into the host cell’s ALB locus). Such constructs may not comprise a promoter that drives the expression of the polypeptide of interest (e.g., multidomain therapeutic protein or GAA fusion protein). For example, the expression of the polypeptide of interest (e.g., multidomain therapeutic protein or GAA fusion protein) can be driven by a promoter of the host cell (e.g., the endogenous ALB promoter when the transgene is integrated into a host cell’s ALB locus). In such cases, the construct may lack control elements (e.g., promoter and/or enhancer) that drive its expression (e.g., a promoterless construct). Nonetheless, in other cases the construct may comprise a promoter and/or enhancer, for example a constitutive promoter or an inducible or tissue-specific (e.g., liver- or platelet-specific) promoter that drives expression of the polypeptide of interest (e.g., multidomain therapeutic protein or GAA fusion protein) in an episome or upon integration. For example, the construct may be a construct for expression (e.g., an episomal construct) but not for insertion. In some embodiments, the construct is not for insertion. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EF1a) promoter, ubiquitin promoters, actin promoters, tubulin promoters, immunoglobulin promoters, a functional fragment thereof, or a combination of any of the foregoing. For example, the promoter may be a CMV promoter or a truncated CMV promoter. In another example, the promoter may be an EF1a promoter. Non-limiting exemplary inducible promoters include those inducible by heat shock, light, chemicals, peptides, metals, steroids, antibiotics, or alcohol. The inducible promoter may be one that has a low basal (non-induced) expression level, such as the Tet-On® promoter (Clontech). Although not required for expression, the constructs may comprise transcriptional or translational regulatory sequences such as promoters, enhancers, insulators, internal ribosome entry sites, additional sequences encoding peptides, and/or polyadenylation signals. The construct may comprise a sequence encoding a polypeptide of interest (e.g., multidomain therapeutic protein or GAA fusion protein) downstream of and operably linked to a signal sequence encoding a signal peptide. In some examples, the nucleic acid construct works in homology-independent insertion of a nucleic acid that encodes a polypeptide of interest (e.g., multidomain therapeutic protein or GAA fusion protein). Such nucleic acid constructs can work, for example, in non-dividing cells (e.g., cells in which non-homologous end joining (NHEJ), not homologous recombination (HR), is the primary mechanism by which double-stranded DNA breaks are repaired) or dividing cells (e.g., actively dividing cells). Such constructs can be, for example, homology-independent donor constructs. In preferred embodiments, promoters and other regulatory sequences are appropriate for use in humans, e.g., recognized by regulatory factors in human cells, e.g., in human liver cells, and acceptable to regulatory authorities for use in humans. Examples of liver-specific promoters include TTR promoters, such as human or mouse TTR promoters. In one example, the construct may comprise a TTR promoter, such as a mouse TTR promoter or a human TTR promoter (e.g., the coding sequence for the polypeptide of interest or multidomain therapeutic protein is operably linked to the TTR promoter). In one example, the construct may comprise a SERPINA1 enhancer, such as a mouse SERPINA1 enhancer or a human SERPINA1 enhancer (e.g., the coding sequence for the polypeptide of interest or multidomain therapeutic protein is operably linked to the SERPINA1 enhancer). In one example, the construct may comprise a TTR promoter and a SERPINA1 enhancer, such as a human SERPINA1 enhancer and a mouse TTR promoter (e.g., the coding sequence for the polypeptide of interest or multidomain therapeutic protein is operably linked to the SERPINA1 enhancer and the TTR promoter).

The constructs disclosed herein can be modified to include or exclude any suitable structural feature as needed for any particular use and/or that confers one or more desired function. For example, some constructs disclosed herein do not comprise a homology arm. Some constructs disclosed herein are capable of insertion into a target genomic locus or a cut site in a target DNA sequence for a nuclease agent (e.g., capable of insertion into a safe harbor gene, such as an ALB locus) by non-homologous end joining. For example, such constructs can be inserted into a blunt end double-strand break following cleavage with a nuclease agent (e.g., CRISPR/Cas system, e.g., a SpyCas9 CRISPR/Cas system) as disclosed herein. In a specific example, the construct can be delivered via AAV and can be capable of insertion by non-homologous end joining (e.g., the construct does not comprise a homology arm).

In a particular example, the construct can be inserted via homology-independent targeted integration. For example, the polypeptide of interest coding sequence (e.g., the multidomain therapeutic protein or GAA fusion protein coding sequence) in the construct can be flanked on each side by a target site for a nuclease agent (e.g., the same target site as in the target DNA sequence for targeted insertion (e.g., in a safe harbor gene), and the same nuclease agent being used to cleave the target DNA sequence for targeted insertion). The nuclease agent can then cleave the target sites flanking the polypeptide of interest coding sequence (e.g., the multidomain therapeutic protein or GAA fusion protein coding sequence). In a specific example, the construct is delivered AAV-mediated delivery, and cleavage of the target sites flanking the polypeptide of interest coding sequence (e.g., the multidomain therapeutic protein or GAA fusion protein coding sequence) can remove the inverted terminal repeats (ITRs) of the AAV. In some instances, the target DNA sequence for targeted insertion (e.g., target DNA sequence in a safe harbor locus such as a gRNA target sequence including the flanking protospacer adjacent motif) is no longer present if the polypeptide of interest coding sequence (e.g., the multidomain therapeutic protein or GAA fusion protein coding sequence) is inserted into the cut site or target DNA sequence in the correct orientation but it is reformed if the polypeptide of interest coding sequence (e.g., the multidomain therapeutic protein or GAA fusion protein coding sequence) is inserted into the cut site or target DNA sequence in the opposite orientation. This can help ensure that the polypeptide of interest coding sequence (e.g., the multidomain therapeutic protein or GAA fusion protein coding sequence) is inserted in the correct orientation for expression.

The constructs disclosed herein can comprise a polyadenylation sequence or polyadenylation tail sequence (e.g., downstream or 3′ of a polypeptide of interest coding sequence). Methods of designing a suitable polyadenylation tail sequence are well-known. The polyadenylation tail sequence can be encoded, for example, as a “poly-A” stretch downstream of the polypeptide of interest coding sequence. A poly-A tail can comprise, for example, at least 20, 30, 40, 50, 60, 70, 80, 90, or 100 adenines, and optionally up to 300 adenines. In a specific example, the poly-A tail comprises 95, 96, 97, 98, 99, or 100 adenine nucleotides. Methods of designing a suitable polyadenylation tail sequence and/or polyadenylation signal sequence are well known. For example, the polyadenylation signal sequence AAUAAA is commonly used in mammalian systems, although variants such as UAUAAA or AU/GUAAA have been identified. See, e.g., Proudfoot (2011) Genes & Dev. 25(17):1770-82, herein incorporated by reference in its entirety for all purposes. The term polyadenylation signal sequence refers to any sequence that directs termination of transcription and addition of a poly-A tail to the mRNA transcript. In eukaryotes, transcription terminators are recognized by protein factors, and termination is followed by polyadenylation, a process of adding a poly(A) tail to the mRNA transcripts in presence of the poly(A) polymerase. The mammalian poly(A) signal typically consists of a core sequence, about 45 nucleotides long, that may be flanked by diverse auxiliary sequences that serve to enhance cleavage and polyadenylation efficiency. The core sequence consists of a highly conserved upstream element (AATAAA or AAUAAA) in the mRNA, referred to as a poly A recognition motif or poly A recognition sequence), recognized by cleavage and polyadenylation-specificity factor (CPSF), and a poorly defined downstream region (rich in Us or Gs and Us), bound by cleavage stimulation factor (CstF). Examples of transcription terminators that can be used include, for example, the human growth hormone (HGH) polyadenylation signal, the simian virus 40 (SV40) late polyadenylation signal, the rabbit beta-globin polyadenylation signal, the bovine growth hormone (BGH) polyadenylation signal, the phosphoglycerate kinase (PGK) polyadenylation signal, an AOX1 transcription termination sequence, a CYC1 transcription termination sequence, or any transcription termination sequence known to be suitable for regulating gene expression in eukaryotic cells. In one example, the polyadenylation signal is a simian virus 40 (SV40) late polyadenylation signal. For example, the polyadenylation signal can comprise, consist essentially of, or consist of SEQ ID NO: 712, 169, or 161. For example, the polyadenylation signal can comprise, consist essentially of, or consist of SEQ ID NO: 169 or 161. For example, the polyadenylation signal can comprise, consist essentially of, or consist of SEQ ID NO: 169. For example, the polyadenylation signal can comprise, consist essentially of, or consist of SEQ ID NO: 712. In another example, the polyadenylation signal is a bovine growth hormone (BGH) polyadenylation signal or a CpG depleted BGH polyadenylation signal. For example, the polyadenylation signal can comprise, consist essentially of, or consist of SEQ ID NO: 162.

The constructs disclosed herein may also comprise splice acceptor sites (e.g., operably linked to the polypeptide of interest coding sequence (e.g., the multidomain therapeutic protein or GAA fusion protein coding sequence), such as upstream or 5′ of the polypeptide of interest coding sequence (e.g., the multidomain therapeutic protein or GAA fusion protein coding sequence)). The splice acceptor site can, for example, comprise NAG or consist of NAG. In a specific example, the splice acceptor is an ALB splice acceptor (e.g., an ALB splice acceptor used in the splicing together of exons 1 and 2 of ALB (i.e., ALB exon 2 splice acceptor)). For example, such a splice acceptor can be derived from the human ALB gene. In another example, the splice acceptor can be derived from the mouse Alb gene (e.g., an ALB splice acceptor used in the splicing together of exons 1 and 2 of mouse Alb (i.e., mouse Alb exon 2 splice acceptor)). In another example, the splice acceptor is a splice acceptor from a gene encoding the polypeptide of interest (e.g., a GAA splice acceptor). For example, such a splice acceptor can be derived from the human GAA gene. Alternatively, such a splice acceptor can be derived from the mouse GAA gene. Additional suitable splice acceptor sites useful in eukaryotes, including artificial splice acceptors, are well-known. See, e.g., Shapiro et al. (1987) Nucleic Acids Res. 15:7155-7174 and Burset et al. (2001) Nucleic Acids Res. 29:255-259, each of which is herein incorporated by reference in its entirety for all purposes. In a specific example, the splice acceptor is a mouse Alb exon 2 splice acceptor. In a specific example, the splice acceptor can comprise, consist essentially of, or consist of SEQ ID NO: 163.

In some examples, the nucleic acid constructs disclosed herein can be bidirectional constructs, which are described in more detail below. In some examples, the nucleic acid constructs disclosed herein can be unidirectional constructs, which are described in more detail below. Likewise, in some examples, the nucleic acid constructs disclosed herein can be in a vector (e.g., viral vector, such as AAV, or rAAV8) and/or a lipid nanoparticle as described in more detail elsewhere herein.

### Polypeptides of Interest and Multidomain Therapeutic Proteins

Any polypeptide of interest may be encoded by the nucleic acid constructs disclosed herein. In one example, the polypeptide of interest is a therapeutic polypeptide (e.g., a polypeptide that is lacking or deficient in a neonatal subject). In one example, the polypeptide of interest is an enzyme.

The polypeptide of interest can be a secreted polypeptide (e.g., a protein that is secreted by the cell and/or is functionally active as a soluble extracellular protein). Alternatively, the polypeptide of interest can be an intracellular polypeptide (e.g., a protein that is not secreted by the cell and is functionally active within the cell, including soluble cytosolic polypeptides).

The polypeptide of interest can be a wild type polypeptide. Alternatively, the polypeptide of interest can be a variant or mutant polypeptide.

In one example, the polypeptide of interest is a liver protein (e.g., a protein that is, endogenously produced in the liver and/or functionally active in the liver). In another example, the polypeptide of interest can be a circulating protein that is produced by the liver. In another example, the polypeptide of interest can be a non-liver protein.

The polypeptide of interest can be an exogenous polypeptide. An “exogenous” polypeptide coding sequence can refer to a coding sequence that has been introduced from an exogenous source to a site within a host cell genome (e.g., at a genomic locus such as a safe harbor locus, including ALB intron 1). That is, the exogenous polypeptide coding sequence is exogenous with respect to its insertion site, and the polypeptide of interest expressed from such an exogenous coding sequence is referred to as an exogenous polypeptide. The exogenous coding sequence can be naturally-occurring or engineered, and can be wild type or a variant. The exogenous coding sequence may include nucleotide sequences other than the sequence that encodes the exogenous polypeptide (e.g., an internal ribosomal entry site). The exogenous coding sequence can be a coding sequence that occurs naturally in the host genome, as a wild type or a variant (e.g., mutant). For example, although the host cell contains the coding sequence of interest (as a wild type or as a variant), the same coding sequence or variant thereof can be introduced as an exogenous source (e.g., for expression at a locus that is highly expressed). The exogenous coding sequence can also be a coding sequence that is not naturally occurring in the host genome, or that expresses an exogenous polypeptide that does not naturally occur in the host genome. An exogenous coding sequence can include an exogenous nucleic acid sequence (e.g., a nucleic acid sequence is not endogenous to the recipient cell), or may be exogenous with respect to its insertion site and/or with respect to its recipient cell.

In one example, the polypeptide of interest is a polypeptide associated with a genetic enzyme deficiency. In certain embodiments, the genetic enzyme deficiency results in infantile onset of disease. In certain embodiments, the genetic enzyme deficiency can be, or routinely is, diagnosed with newborn screening. In certain embodiments, the enzyme deficiency may manifest in various severity of disease such that the age of onset may include an infantile onset form of the disease and a later onset form of the disease (e.g., childhood, adolescent, or adult form of onset).

In one example, the polypeptide of interest is a lysosomal alpha-glucosidase (GAA) polypeptide.

In another example, the polypeptide of interest is a multidomain therapeutic protein. A multidomain therapeutic protein as described herein includes a lysosomal alpha-glucosidase (GAA; e.g., to provide GAA enzyme replacement activity) linked to or fused to a delivery domain that provides binding to an internalization effector (a protein that is capable of being internalized into a cell or that otherwise participates in or contributes to retrograde membrane trafficking). Examples of multidomain therapeutic proteins can be found in WO 2013/138400, WO 2017/007796, WO 2017/190079, WO 2017/100467, WO 2018/226861, WO 2019/157224, and WO 2019/222663, each of which is herein incorporated by reference in its entirety for all purposes. For example, the multidomain therapeutic proteins described herein can comprise a CD63-binding delivery domain linked to or fused to a lysosomal alpha-glucosidase (GAA). CD63-binding domains and GAA are described in more detail below. The CD63-binding domain provides binding to the internalization factor CD63. The multidomain therapeutic protein is targeted to the muscle by targeting CD63, which is a rapidly internalizing protein highly expressed in the muscle. In some multidomain therapeutic proteins, the CD63-binding delivery domain is covalently linked to the GAA. The covalent linkage may be any type of covalent bond (i.e., any bond that involved sharing of electrons). In some cases, the covalent bond is a peptide bond between two amino acids, such that the GAA and the CD63-binding delivery domain in whole or in part form a continuous polypeptide chain, as in a fusion protein. In some cases, the GAA portion and the CD63-binding delivery domain portion are directly linked. In other cases, a linker, such as a peptide linker, is used to tether the two portions. Any suitable linker can be used. See Chen et al., “Fusion protein linkers: property, design and functionality,” 65(10) Adv Drug Deliv Rev. 1357-69 (2013). In some cases, a cleavable linker is used. For example, a cathepsin cleavable linker can be inserted between the CD63-binding delivery domain and the GAA to facilitate removal of the CD63-binding delivery domain in the lysosome. In another example, the linker can comprise an amino acid sequence, e.g., about 10 amino acids in length, for example, 1, 2, 3, 4, 5, 6, 7, 8, 8, or 10 repeats of Gly₄Ser (SEQ ID NO: 600). In one example, the linker comprises, consists essentially of, or consists of three such repeats (SEQ ID NO: 713). For example, the coding sequence for the linker can comprise, consist essentially of, or consist of any one of SEQ ID NOS: 715-719. In another example, the linker comprises, consists essentially of, or consists of two such repeats (SEQ ID NO: 714). For example, the coding sequence for the linker can comprise, consist essentially of, or consist of any one of SEQ ID NOS: 720-726. In another example, the linker comprises, consists essentially of, or consists of one such repeat (SEQ ID NO: 600). For example, the coding sequence for the linker can comprise, consist essentially of, or consist of SEQ ID NO: 727.

In a particular multidomain therapeutic protein, the GAA is covalently linked to the C-terminus of the heavy chain of an anti-CD63 antibody or to the C-terminus of the light chain. In another particular multidomain therapeutic protein, the GAA is covalently linked to the N-terminus of the heavy chain of an anti-CD63 antibody or to the N-terminus of the light chain. In another particular embodiment, the GAA is linked to the C-terminus of an anti-CD63 scFv domain.

As another example, the multidomain therapeutic proteins described herein can comprise a TfR-binding delivery domain linked to or fused to a lysosomal alpha-glucosidase (GAA). TfR-binding domains and GAA are described in more detail below. The TfR-binding domain provides binding to the internalization factor TfR. The multidomain therapeutic protein produced by the liver is targeted the muscle and CNS by targeting TfR, which is expressed in muscle and on brain endothelial cells. Transcytosis of TfR in these cells enables blood-brain-barrier crossing. In some multidomain therapeutic proteins, the TfR-binding delivery domain is covalently linked to the GAA. The covalent linkage may be any type of covalent bond (i.e., any bond that involved sharing of electrons). In some cases, the covalent bond is a peptide bond between two amino acids, such that the GAA and the TfR-binding delivery domain in whole or in part form a continuous polypeptide chain, as in a fusion protein. In some cases, the GAA portion and the TfR-binding delivery domain portion are directly linked. In other cases, a linker, such as a peptide linker, is used to tether the two portions. Any suitable linker can be used. See Chen et al., “Fusion protein linkers: property, design and functionality,” 65(10) Adv Drug Deliv Rev. 1357-69 (2013). In some cases, a cleavable linker is used. For example, a cathepsin cleavable linker can be inserted between the TfR-binding delivery domain and the GAA to facilitate removal of the TfR-binding delivery domain in the lysosome. In another example, the linker can comprise an amino acid sequence, e.g., about 10 amino acids in length, for example, 1, 2, 3, 4, 5, 6, 7, 8, 8, or 10 repeats of Gly₄Ser (SEQ ID NO: 600). In one example, the linker comprises, consists essentially of, or consists of three such repeats (SEQ ID NO: 713). For example, the coding sequence for the linker can comprise, consist essentially of, or consist of any one of SEQ ID NOS: 715-719. In another example, the linker comprises, consists essentially of, or consists of two such repeats (SEQ ID NO: 714). For example, the coding sequence for the linker can comprise, consist essentially of, or consist of any one of SEQ ID NOS: 720-726. In another example, the linker comprises, consists essentially of, or consists of one such repeat (SEQ ID NO: 600). For example, the coding sequence for the linker can comprise, consist essentially of, or consist of SEQ ID NO: 727.

In a particular multidomain therapeutic protein, the GAA is covalently linked to the C-terminus of the heavy chain of an anti-TfR antibody or to the C-terminus of the light chain. In another particular multidomain therapeutic protein, the GAA is covalently linked to the N-terminus of the heavy chain of an anti-TfR antibody or to the N-terminus of the light chain. In another particular embodiment, the GAA is linked to the C-terminus of an anti-TfR scFv domain.

### (A) Lysosomal Alpha-Glucosidase (GAA)

Lysosomal alpha-glucosidase (GAA; also known as acid alpha-glucosidase, acid alpha-glucosidase preproprotein, acid maltase, aglucosidase alfa, alpha-1,4-glucosidase, amyloglucosidase, glucoamylase, LYAG) is encoded by GAA. This enzyme is active in lysosomes, where it breaks down glycogen into glucose.

The human GAA gene (NCBI GenelD 2548) encodes a 952 amino acid protein. In the lysosome, human GAA is sequentially processed by proteases to polypeptides of 76-, 19.4-, and 3.9-kDa that remain associated. Further cleavage between R(200) and A(204) inefficiently converts the 76-kDa polypeptide to the mature 70-kDa form with an additional 10.4-kDa polypeptide. GAA maturation increases its affinity for glycogen by 7-10 fold. A signal peptide is encoded by amino acids 1-27, a propeptide encoded by amino acids 28-69, lysosomal alpha-glucosidase after removal of the signal peptide and propeptide is encoded by amino acids 70-952, the 76 kDa lysosomal alpha-glucosidase is encoded by amino acids 123-952, and the 70 kDa lysosomal alpha-glucosidase is encoded by amino acids 204-952.

The GAA expressed from the compositions and methods disclosed herein can be any wild type or variant GAA. In one example, the GAA is a human GAA protein. Human GAA is assigned UniProt reference number P10253. An exemplary amino acid sequence for human GAA is assigned NCBI Accession No. NP_000143.2 and is set forth in SEQ ID NO: 170. An exemplary human GAA mRNA (cDNA) sequence is assigned NCBI Accession No. NM_000152.5 and is set forth in SEQ ID NO: 171. An exemplary human GAA coding sequence is assigned CCDS ID CCDS32760.1 and is set forth in SEQ ID NO: 172. An exemplary mature human GAA amino acid sequence (i.e., the human GAA sequence after removal of the signal peptide and propeptide) starting at amino acid 70 (i.e., GAA 70-952) is set forth in SEQ ID NO: 173. An exemplary coding sequence for GAA 70-952 is set forth in SEQ ID NO: 174.

In some examples, the GAA (e.g., human GAA) is a wild type GAA (e.g., wild type human GAA) sequence or a fragment thereof. For example, the GAA can be a fragment comprising the mature GAA amino acid sequence (i.e., the GAA sequence after removal of the signal peptide and propeptide), a fragment comprising the 77 kDa form of GAA, or a fragment comprising the 70 kDa form of GAA. In a specific example, the GAA can comprise SEQ ID NO: 173 or can be at least 90%, at least 91%, at least 92%, at least 93%, at least 94%, at least 95%, at least 96%, at least 97%, at least 98%, at least 99%, or at least 99.5% identical to SEQ ID NO: 173. In another specific example, the GAA can consist essentially of SEQ ID NO: 173. In another specific example, the GAA can consist of SEQ ID NO: 173.

The GAA coding sequences in the constructs disclosed herein may include one or more modifications such as codon optimization (e.g., to human codons), depletion of CpG dinucleotides, mutation of cryptic splice sites, addition of one or more glycosylation sites, or any combination thereof. CpG dinucleotides in a construct can limit the therapeutic utility of the construct. First, unmethylated CpG dinucleotides can interact with host toll-like receptor-9 (TLR-9) to stimulate innate, proinflammatory immune responses. Second, once the CpG dinucleotides become methylated, they can result in the suppression of transgene expression coordinated by methyl-CpG binding proteins. Cryptic splice sites are sequences in a pre-messenger RNA that are not normally used as splice sites, but that can be activated, for example, by mutations that either inactivate canonical splice sites or create splice sites where one did not exist before. Accurate splice site selection is critical for successful gene expression, and removal of cryptic splice sites can favor use of the normal or intended splice site.

In one example, a GAA coding sequence in a construct disclosed herein has one or more cryptic splice sites mutated or removed. In another example, a GAA coding sequence in a construct disclosed herein has all identified cryptic splice sites mutated or removed. In another example, a GAA coding sequence in a construct disclosed herein has one or more CpG dinucleotides removed (i.e., is CpG depleted). In another example, a GAA coding sequence in a construct disclosed herein has all CpG dinucleotides removed (i.e., is fully CpG depleted). In another example, a GAA coding sequence in a construct disclosed herein is codon optimized (e.g., codon optimized for expression in a human or mammal). In a specific example, a GAA coding sequence in a construct disclosed herein has one or more CpG dinucleotides removed (i.e., is CpG depleted) and has one or more cryptic splice sites mutated or removed. In another specific example, a GAA coding sequence in a construct disclosed herein has all CpG dinucleotides removed and has one or more or all identified cryptic splice sites mutated or removed. In another specific example, a GAA coding sequence in a construct disclosed herein has one or more CpG dinucleotides removed (i.e., is CpG depleted) and is codon optimized (e.g., codon optimized for expression in a human or mammal). In another specific example, a GAA coding sequence in a construct disclosed herein has all CpG dinucleotides removed (i.e., is fully CpG depleted) and is codon optimized (e.g., codon optimized for expression in a human or mammal).

Various GAA coding sequences are provided. In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 174-182 and 205-212. In another example, the GAA coding sequence is (or comprises a sequence) at least 95%, at least 96%, at least 97%, at least 98%, at least 99%, at least 99.5%, or 100% identical to any one of SEQ ID NOS: 174-182 and 205-212. In another example, the GAA coding sequence is (or comprises a sequence) at least 99%, at least 99.5%, or 100% identical to any one of SEQ ID NOS: 174-182 and 205-212. In another example, the GAA coding sequence comprises the sequence set forth in any one of SEQ ID NOS: 174-182 and 205-212. In another example, the GAA coding sequence consists essentially of the sequence set forth in any one of SEQ ID NOS: 174-182 and 205-212. In another example, the GAA coding sequence consists of the sequence set forth in any one of SEQ ID NOS: 174-182 and 205-212. Various GAA coding sequences are provided. In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 174-182. In another example, the GAA coding sequence is (or comprises a sequence) at least 95%, at least 96%, at least 97%, at least 98%, at least 99%, at least 99.5%, or 100% identical to any one of SEQ ID NOS: 174-182. In another example, the GAA coding sequence is (or comprises a sequence) at least 99%, at least 99.5%, or 100% identical to any one of SEQ ID NOS: 174-182. In another example, the GAA coding sequence comprises the sequence set forth in any one of SEQ ID NOS: 174-182. In another example, the GAA coding sequence consists essentially of the sequence set forth in any one of SEQ ID NOS: 174-182. In another example, the GAA coding sequence consists of the sequence set forth in any one of SEQ ID NOS: 174-182. In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 176. In another example, the GAA coding sequence is (or comprises a sequence) at least 95%, at least 96%, at least 97%, at least 98%, at least 99%, at least 99.5%, or 100% identical to SEQ ID NO: 176. In another example, the GAA coding sequence is (or comprises a sequence) at least 99%, at least 99.5%, or 100% identical to SEQ ID NO: 176. In another example, the GAA coding sequence comprises the sequence set forth in SEQ ID NO: 176. In another example, the GAA coding sequence consists essentially of the sequence set forth in SEQ ID NO: 176. In another example, the GAA coding sequence consists of the sequence set forth in SEQ ID NO: 176. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

Various codon optimized GAA coding sequences are provided. The GAA coding sequence can be, for example, CpG-depleted (e.g., fully CpG depleted) and/or codon optimized (e.g., CpG depleted (e.g., fully CpG-depleted) and codon optimized). In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 175-182. In another example, the GAA coding sequence is (or comprises a sequence) at least 95%, at least 96%, at least 97%, at least 98%, at least 99%, at least 99.5%, or 100% identical to any one of SEQ ID NOS: 175-182. In another example, the GAA coding sequence is (or comprises a sequence) at least 99%, at least 99.5%, or 100% identical to any one of SEQ ID NOS: 175-182. In another example, the GAA coding sequence comprises the sequence set forth in any one of SEQ ID NOS: 175-182. In another example, the GAA coding sequence consists essentially of the sequence set forth in any one of SEQ ID NOS: 175-182. In another example, the GAA coding sequence consists of the sequence set forth in any one of SEQ ID NOS: 175-182. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 176. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 176 and encodes a GAA protein (or a GAA protein comprising a sequence) at least 99%, at least 99.5%, or 100% identical to SEQ ID NO: 173. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 176 and encodes a GAA protein comprising the sequence set forth in SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 176. In another example, the GAA coding sequence is (or comprises a sequence) at least 95%, at least 96%, at least 97%, at least 98%, at least 99%, at least 99.5%, or 100% identical to SEQ ID NO: 176 and encodes a GAA protein (or a GAA protein comprising a sequence) at least 99%, at least 99.5%, or 100% identical to SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 176 and encodes a GAA protein comprising the sequence set forth in SEQ ID NO: 173. In another example, the GAA coding sequence is (or comprises a sequence) at least 99%, at least 99.5%, or 100% identical to SEQ ID NO: 176. In another example, the GAA coding sequence is (or comprises a sequence) at least 99%, at least 99.5%, or 100% identical to SEQ ID NO: 176 and encodes a GAA protein (or a GAA protein comprising a sequence) at least 99%, at least 99.5%, or 100% identical to SEQ ID NO: 173. In another example, the GAA coding sequence is (or comprises a sequence) at least 99%, at least 99.5%, or 100% identical to SEQ ID NO: 176 and encodes a GAA protein comprising the sequence set forth in SEQ ID NO: 173. In another example, the GAA coding sequence comprises the sequence set forth in SEQ ID NO: 176. In another example, the GAA coding sequence consists essentially of the sequence set forth in SEQ ID NO: 176. In another example, the GAA coding sequence consists of the sequence set forth in SEQ ID NO: 176. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 174. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 174 and encodes a GAA protein (or a GAA protein comprising a sequence) at least 99%, at least 99.5%, or 100% identical to SEQ ID NO: 173. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 174 and encodes a GAA protein comprising the sequence set forth in SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 174. In another example, the GAA coding sequence is (or comprises a sequence) at least 95%, at least 96%, at least 97%, at least 98%, at least 99%, at least 99.5%, or 100% identical to SEQ ID NO: 174 and encodes a GAA protein (or a GAA protein comprising a sequence) at least 99%, at least 99.5%, or 100% identical to SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 174 and encodes a GAA protein comprising the sequence set forth in SEQ ID NO: 173. In another example, the GAA coding sequence is (or comprises a sequence) at least 99%, at least 99.5%, or 100% identical to SEQ ID NO: 174. In another example, the GAA coding sequence is (or comprises a sequence) at least 99%, at least 99.5%, or 100% identical to SEQ ID NO: 174 and encodes a GAA protein (or a GAA protein comprising a sequence) at least 99%, at least 99.5%, or 100% identical to SEQ ID NO: 173. In another example, the GAA coding sequence is (or comprises a sequence) at least 99%, at least 99.5%, or 100% identical to SEQ ID NO: 174 and encodes a GAA protein comprising the sequence set forth in SEQ ID NO: 173. In another example, the GAA coding sequence comprises the sequence set forth in SEQ ID NO: 174. In another example, the GAA coding sequence consists essentially of the sequence set forth in SEQ ID NO: 174. In another example, the GAA coding sequence consists of the sequence set forth in SEQ ID NO: 174. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 181. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 181 and encodes a GAA protein (or a GAA protein comprising a sequence) at least 99%, at least 99.5%, or 100% identical to SEQ ID NO: 173. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 181 and encodes a GAA protein comprising the sequence set forth in SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 181. In another example, the GAA coding sequence is (or comprises a sequence) at least 95%, at least 96%, at least 97%, at least 98%, at least 99%, at least 99.5%, or 100% identical to SEQ ID NO: 181 and encodes a GAA protein (or a GAA protein comprising a sequence) at least 99%, at least 99.5%, or 100% identical to SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 181 and encodes a GAA protein comprising the sequence set forth in SEQ ID NO: 173. In another example, the GAA coding sequence is (or comprises a sequence) at least 99%, at least 99.5%, or 100% identical to SEQ ID NO: 181. In another example, the GAA coding sequence is (or comprises a sequence) at least 99%, at least 99.5%, or 100% identical to SEQ ID NO: 181 and encodes a GAA protein (or a GAA protein comprising a sequence) at least 99%, at least 99.5%, or 100% identical to SEQ ID NO: 173. In another example, the GAA coding sequence is (or comprises a sequence) at least 99%, at least 99.5%, or 100% identical to SEQ ID NO: 181 and encodes a GAA protein comprising the sequence set forth in SEQ ID NO: 173. In another example, the GAA coding sequence comprises the sequence set forth in SEQ ID NO: 181. In another example, the GAA coding sequence consists essentially of the sequence set forth in SEQ ID NO: 181. In another example, the GAA coding sequence consists of the sequence set forth in SEQ ID NO: 181. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 180. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 180 and encodes a GAA protein (or a GAA protein comprising a sequence) at least 99%, at least 99.5%, or 100% identical to SEQ ID NO: 173. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 180 and encodes a GAA protein comprising the sequence set forth in SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 180. In another example, the GAA coding sequence is (or comprises a sequence) at least 95%, at least 96%, at least 97%, at least 98%, at least 99%, at least 99.5%, or 100% identical to SEQ ID NO: 180 and encodes a GAA protein (or a GAA protein comprising a sequence) at least 99%, at least 99.5%, or 100% identical to SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 180 and encodes a GAA protein comprising the sequence set forth in SEQ ID NO: 173. In another example, the GAA coding sequence is (or comprises a sequence) at least 99%, at least 99.5%, or 100% identical to SEQ ID NO: 180. In another example, the GAA coding sequence is (or comprises a sequence) at least 99%, at least 99.5%, or 100% identical to SEQ ID NO: 180 and encodes a GAA protein (or a GAA protein comprising a sequence) at least 99%, at least 99.5%, or 100% identical to SEQ ID NO: 173. In another example, the GAA coding sequence is (or comprises a sequence) at least 99%, at least 99.5%, or 100% identical to SEQ ID NO: 180 and encodes a GAA protein comprising the sequence set forth in SEQ ID NO: 173. In another example, the GAA coding sequence comprises the sequence set forth in SEQ ID NO: 180. In another example, the GAA coding sequence consists essentially of the sequence set forth in SEQ ID NO: 180. In another example, the GAA coding sequence consists of the sequence set forth in SEQ ID NO: 180. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 178. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 178 and encodes a GAA protein (or a GAA protein comprising a sequence) at least 99%, at least 99.5%, or 100% identical to SEQ ID NO: 173. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 178 and encodes a GAA protein comprising the sequence set forth in SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 178. In another example, the GAA coding sequence is (or comprises a sequence) at least 95%, at least 96%, at least 97%, at least 98%, at least 99%, at least 99.5%, or 100% identical to SEQ ID NO: 178 and encodes a GAA protein (or a GAA protein comprising a sequence) at least 99%, at least 99.5%, or 100% identical to SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 178 and encodes a GAA protein comprising the sequence set forth in SEQ ID NO: 173. In another example, the GAA coding sequence is (or comprises a sequence) at least 99%, at least 99.5%, or 100% identical to SEQ ID NO: 178. In another example, the GAA coding sequence is (or comprises a sequence) at least 99%, at least 99.5%, or 100% identical to SEQ ID NO: 178 and encodes a GAA protein (or a GAA protein comprising a sequence) at least 99%, at least 99.5%, or 100% identical to SEQ ID NO: 173. In another example, the GAA coding sequence is (or comprises a sequence) at least 99%, at least 99.5%, or 100% identical to SEQ ID NO: 178 and encodes a GAA protein comprising the sequence set forth in SEQ ID NO: 173. In another example, the GAA coding sequence comprises the sequence set forth in SEQ ID NO: 178. In another example, the GAA coding sequence consists essentially of the sequence set forth in SEQ ID NO: 178. In another example, the GAA coding sequence consists of the sequence set forth in SEQ ID NO: 178. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

Various other GAA coding sequences are provided. In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 174 and 205-212. In another example, the GAA coding sequence is (or comprises a sequence) at least 95%, at least 96%, at least 97%, at least 98%, at least 99%, at least 99.5%, or 100% identical to any one of SEQ ID NOS: 174 and 205-212. In another example, the GAA coding sequence is (or comprises a sequence) at least 99%, at least 99.5%, or 100% identical to any one of SEQ ID NOS: 174 and 205-212. In another example, the GAA coding sequence comprises the sequence set forth in any one of SEQ ID NOS: 174 and 205-212. In another example, the GAA coding sequence consists essentially of the sequence set forth in any one of SEQ ID NOS: 174 and 205-212. In another example, the GAA coding sequence consists of the sequence set forth in any one of SEQ ID NOS: 174 and 205-212. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

Various other codon optimized GAA coding sequences are provided. The GAA coding sequence can be, for example, CpG-depleted (e.g., fully CpG depleted) and/or codon optimized (e.g., CpG depleted (e.g., fully CpG-depleted) and codon optimized). In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 205-212. In another example, the GAA coding sequence is (or comprises a sequence) at least 95%, at least 96%, at least 97%, at least 98%, at least 99%, at least 99.5%, or 100% identical to any one of SEQ ID NOS: 205-212. In another example, the GAA coding sequence is (or comprises a sequence) at least 99%, at least 99.5%, or 100% identical to any one of SEQ ID NOS: 205-212. In another example, the GAA coding sequence comprises the sequence set forth in any one of SEQ ID NOS: 205-212. In another example, the GAA coding sequence consists essentially of the sequence set forth in any one of SEQ ID NOS: 205-212. In another example, the GAA coding sequence consists of the sequence set forth in any one of SEQ ID NOS: 205-212. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 176. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 176 and encodes a GAA protein (or a GAA protein comprising a sequence) at least 99%, at least 99.5%, or 100% identical to SEQ ID NO: 173. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 176 and encodes a GAA protein comprising the sequence set forth in SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 176. In another example, the GAA coding sequence is (or comprises a sequence) at least 95%, at least 96%, at least 97%, at least 98%, at least 99%, at least 99.5%, or 100% identical to SEQ ID NO: 176 and encodes a GAA protein (or a GAA protein comprising a sequence) at least 99%, at least 99.5%, or 100% identical to SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 176 and encodes a GAA protein comprising the sequence set forth in SEQ ID NO: 173. In another example, the GAA coding sequence is (or comprises a sequence) at least 99%, at least 99.5%, or 100% identical to SEQ ID NO: 176. In another example, the GAA coding sequence is (or comprises a sequence) at least 99%, at least 99.5%, or 100% identical to SEQ ID NO: 176 and encodes a GAA protein (or a GAA protein comprising a sequence) at least 99%, at least 99.5%, or 100% identical to SEQ ID NO: 173. In another example, the GAA coding sequence is (or comprises a sequence) at least 99%, at least 99.5%, or 100% identical to SEQ ID NO: 176 and encodes a GAA protein comprising the sequence set forth in SEQ ID NO: 173. In another example, the GAA coding sequence comprises the sequence set forth in SEQ ID NO: 176. In another example, the GAA coding sequence consists essentially of the sequence set forth in SEQ ID NO: 176. In another example, the GAA coding sequence consists of the sequence set forth in SEQ ID NO: 176. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 174. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 174 and encodes a GAA protein (or a GAA protein comprising a sequence) at least 99%, at least 99.5%, or 100% identical to SEQ ID NO: 173. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 174 and encodes a GAA protein comprising the sequence set forth in SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 174. In another example, the GAA coding sequence is (or comprises a sequence) at least 95%, at least 96%, at least 97%, at least 98%, at least 99%, at least 99.5%, or 100% identical to SEQ ID NO: 174 and encodes a GAA protein (or a GAA protein comprising a sequence) at least 99%, at least 99.5%, or 100% identical to SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 174 and encodes a GAA protein comprising the sequence set forth in SEQ ID NO: 173. In another example, the GAA coding sequence is (or comprises a sequence) at least 99%, at least 99.5%, or 100% identical to SEQ ID NO: 174. In another example, the GAA coding sequence is (or comprises a sequence) at least 99%, at least 99.5%, or 100% identical to SEQ ID NO: 174 and encodes a GAA protein (or a GAA protein comprising a sequence) at least 99%, at least 99.5%, or 100% identical to SEQ ID NO: 173. In another example, the GAA coding sequence is (or comprises a sequence) at least 99%, at least 99.5%, or 100% identical to SEQ ID NO: 174 and encodes a GAA protein comprising the sequence set forth in SEQ ID NO: 173. In another example, the GAA coding sequence comprises the sequence set forth in SEQ ID NO: 174. In another example, the GAA coding sequence consists essentially of the sequence set forth in SEQ ID NO: 174. In another example, the GAA coding sequence consists of the sequence set forth in SEQ ID NO: 174. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 205. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 205 and encodes a GAA protein (or a GAA protein comprising a sequence) at least 99%, at least 99.5%, or 100% identical to SEQ ID NO: 173. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 205 and encodes a GAA protein comprising the sequence set forth in SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 205. In another example, the GAA coding sequence is (or comprises a sequence) at least 95%, at least 96%, at least 97%, at least 98%, at least 99%, at least 99.5%, or 100% identical to SEQ ID NO: 205 and encodes a GAA protein (or a GAA protein comprising a sequence) at least 99%, at least 99.5%, or 100% identical to SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 205 and encodes a GAA protein comprising the sequence set forth in SEQ ID NO: 173. In another example, the GAA coding sequence is (or comprises a sequence) at least 99%, at least 99.5%, or 100% identical to SEQ ID NO: 205. In another example, the GAA coding sequence is (or comprises a sequence) at least 99%, at least 99.5%, or 100% identical to SEQ ID NO: 205 and encodes a GAA protein (or a GAA protein comprising a sequence) at least 99%, at least 99.5%, or 100% identical to SEQ ID NO: 173. In another example, the GAA coding sequence is (or comprises a sequence) at least 99%, at least 99.5%, or 100% identical to SEQ ID NO: 205 and encodes a GAA protein comprising the sequence set forth in SEQ ID NO: 173. In another example, the GAA coding sequence comprises the sequence set forth in SEQ ID NO: 205. In another example, the GAA coding sequence consists essentially of the sequence set forth in SEQ ID NO: 205. In another example, the GAA coding sequence consists of the sequence set forth in SEQ ID NO: 205. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 206. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 206 and encodes a GAA protein (or a GAA protein comprising a sequence) at least 99%, at least 99.5%, or 100% identical to SEQ ID NO: 173. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 206 and encodes a GAA protein comprising the sequence set forth in SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 206. In another example, the GAA coding sequence is (or comprises a sequence) at least 95%, at least 96%, at least 97%, at least 98%, at least 99%, at least 99.5%, or 100% identical to SEQ ID NO: 206 and encodes a GAA protein (or a GAA protein comprising a sequence) at least 99%, at least 99.5%, or 100% identical to SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 206 and encodes a GAA protein comprising the sequence set forth in SEQ ID NO: 173. In another example, the GAA coding sequence is (or comprises a sequence) at least 99%, at least 99.5%, or 100% identical to SEQ ID NO: 206. In another example, the GAA coding sequence is (or comprises a sequence) at least 99%, at least 99.5%, or 100% identical to SEQ ID NO: 206 and encodes a GAA protein (or a GAA protein comprising a sequence) at least 99%, at least 99.5%, or 100% identical to SEQ ID NO: 173. In another example, the GAA coding sequence is (or comprises a sequence) at least 99%, at least 99.5%, or 100% identical to SEQ ID NO: 206 and encodes a GAA protein comprising the sequence set forth in SEQ ID NO: 173. In another example, the GAA coding sequence comprises the sequence set forth in SEQ ID NO: 206. In another example, the GAA coding sequence consists essentially of the sequence set forth in SEQ ID NO: 206. In another example, the GAA coding sequence consists of the sequence set forth in SEQ ID NO: 206. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 207. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 207 and encodes a GAA protein (or a GAA protein comprising a sequence) at least 99%, at least 99.5%, or 100% identical to SEQ ID NO: 173. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 207 and encodes a GAA protein comprising the sequence set forth in SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 207. In another example, the GAA coding sequence is (or comprises a sequence) at least 95%, at least 96%, at least 97%, at least 98%, at least 99%, at least 99.5%, or 100% identical to SEQ ID NO: 207 and encodes a GAA protein (or a GAA protein comprising a sequence) at least 99%, at least 99.5%, or 100% identical to SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 207 and encodes a GAA protein comprising the sequence set forth in SEQ ID NO: 173. In another example, the GAA coding sequence is (or comprises a sequence) at least 99%, at least 99.5%, or 100% identical to SEQ ID NO: 207. In another example, the GAA coding sequence is (or comprises a sequence) at least 99%, at least 99.5%, or 100% identical to SEQ ID NO: 207 and encodes a GAA protein (or a GAA protein comprising a sequence) at least 99%, at least 99.5%, or 100% identical to SEQ ID NO: 173. In another example, the GAA coding sequence is (or comprises a sequence) at least 99%, at least 99.5%, or 100% identical to SEQ ID NO: 207 and encodes a GAA protein comprising the sequence set forth in SEQ ID NO: 173. In another example, the GAA coding sequence comprises the sequence set forth in SEQ ID NO: 207. In another example, the GAA coding sequence consists essentially of the sequence set forth in SEQ ID NO: 207. In another example, the GAA coding sequence consists of the sequence set forth in SEQ ID NO: 207. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 208. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 208 and encodes a GAA protein (or a GAA protein comprising a sequence) at least 99%, at least 99.5%, or 100% identical to SEQ ID NO: 173. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 208 and encodes a GAA protein comprising the sequence set forth in SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 208. In another example, the GAA coding sequence is (or comprises a sequence) at least 95%, at least 96%, at least 97%, at least 98%, at least 99%, at least 99.5%, or 100% identical to SEQ ID NO: 208 and encodes a GAA protein (or a GAA protein comprising a sequence) at least 99%, at least 99.5%, or 100% identical to SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 208 and encodes a GAA protein comprising the sequence set forth in SEQ ID NO: 173. In another example, the GAA coding sequence is (or comprises a sequence) at least 99%, at least 99.5%, or 100% identical to SEQ ID NO: 208. In another example, the GAA coding sequence is (or comprises a sequence) at least 99%, at least 99.5%, or 100% identical to SEQ ID NO: 208 and encodes a GAA protein (or a GAA protein comprising a sequence) at least 99%, at least 99.5%, or 100% identical to SEQ ID NO: 173. In another example, the GAA coding sequence is (or comprises a sequence) at least 99%, at least 99.5%, or 100% identical to SEQ ID NO: 208 and encodes a GAA protein comprising the sequence set forth in SEQ ID NO: 173. In another example, the GAA coding sequence comprises the sequence set forth in SEQ ID NO: 208. In another example, the GAA coding sequence consists essentially of the sequence set forth in SEQ ID NO: 208. In another example, the GAA coding sequence consists of the sequence set forth in SEQ ID NO: 208. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 209. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 209 and encodes a GAA protein (or a GAA protein comprising a sequence) at least 99%, at least 99.5%, or 100% identical to SEQ ID NO: 173. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 209 and encodes a GAA protein comprising the sequence set forth in SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 209. In another example, the GAA coding sequence is (or comprises a sequence) at least 95%, at least 96%, at least 97%, at least 98%, at least 99%, at least 99.5%, or 100% identical to SEQ ID NO: 209 and encodes a GAA protein (or a GAA protein comprising a sequence) at least 99%, at least 99.5%, or 100% identical to SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 209 and encodes a GAA protein comprising the sequence set forth in SEQ ID NO: 173. In another example, the GAA coding sequence is (or comprises a sequence) at least 99%, at least 99.5%, or 100% identical to SEQ ID NO: 209. In another example, the GAA coding sequence is (or comprises a sequence) at least 99%, at least 99.5%, or 100% identical to SEQ ID NO: 209 and encodes a GAA protein (or a GAA protein comprising a sequence) at least 99%, at least 99.5%, or 100% identical to SEQ ID NO: 173. In another example, the GAA coding sequence is (or comprises a sequence) at least 99%, at least 99.5%, or 100% identical to SEQ ID NO: 209 and encodes a GAA protein comprising the sequence set forth in SEQ ID NO: 173. In another example, the GAA coding sequence comprises the sequence set forth in SEQ ID NO: 209. In another example, the GAA coding sequence consists essentially of the sequence set forth in SEQ ID NO: 209. In another example, the GAA coding sequence consists of the sequence set forth in SEQ ID NO: 209. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 210. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 210 and encodes a GAA protein (or a GAA protein comprising a sequence) at least 99%, at least 99.5%, or 100% identical to SEQ ID NO: 173. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 210 and encodes a GAA protein comprising the sequence set forth in SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 210. In another example, the GAA coding sequence is (or comprises a sequence) at least 95%, at least 96%, at least 97%, at least 98%, at least 99%, at least 99.5%, or 100% identical to SEQ ID NO: 210 and encodes a GAA protein (or a GAA protein comprising a sequence) at least 99%, at least 99.5%, or 100% identical to SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 210 and encodes a GAA protein comprising the sequence set forth in SEQ ID NO: 173. In another example, the GAA coding sequence is (or comprises a sequence) at least 99%, at least 99.5%, or 100% identical to SEQ ID NO: 210. In another example, the GAA coding sequence is (or comprises a sequence) at least 99%, at least 99.5%, or 100% identical to SEQ ID NO: 210 and encodes a GAA protein (or a GAA protein comprising a sequence) at least 99%, at least 99.5%, or 100% identical to SEQ ID NO: 173. In another example, the GAA coding sequence is (or comprises a sequence) at least 99%, at least 99.5%, or 100% identical to SEQ ID NO: 210 and encodes a GAA protein comprising the sequence set forth in SEQ ID NO: 173. In another example, the GAA coding sequence comprises the sequence set forth in SEQ ID NO: 210. In another example, the GAA coding sequence consists essentially of the sequence set forth in SEQ ID NO: 210. In another example, the GAA coding sequence consists of the sequence set forth in SEQ ID NO: 210. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 211. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 211 and encodes a GAA protein (or a GAA protein comprising a sequence) at least 99%, at least 99.5%, or 100% identical to SEQ ID NO: 173. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 211 and encodes a GAA protein comprising the sequence set forth in SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 211. In another example, the GAA coding sequence is (or comprises a sequence) at least 95%, at least 96%, at least 97%, at least 98%, at least 99%, at least 99.5%, or 100% identical to SEQ ID NO: 211 and encodes a GAA protein (or a GAA protein comprising a sequence) at least 99%, at least 99.5%, or 100% identical to SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 211 and encodes a GAA protein comprising the sequence set forth in SEQ ID NO: 173. In another example, the GAA coding sequence is (or comprises a sequence) at least 99%, at least 99.5%, or 100% identical to SEQ ID NO: 211. In another example, the GAA coding sequence is (or comprises a sequence) at least 99%, at least 99.5%, or 100% identical to SEQ ID NO: 211 and encodes a GAA protein (or a GAA protein comprising a sequence) at least 99%, at least 99.5%, or 100% identical to SEQ ID NO: 173. In another example, the GAA coding sequence is (or comprises a sequence) at least 99%, at least 99.5%, or 100% identical to SEQ ID NO: 211 and encodes a GAA protein comprising the sequence set forth in SEQ ID NO: 173. In another example, the GAA coding sequence comprises the sequence set forth in SEQ ID NO: 211. In another example, the GAA coding sequence consists essentially of the sequence set forth in SEQ ID NO: 211. In another example, the GAA coding sequence consists of the sequence set forth in SEQ ID NO: 211. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 212. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 212 and encodes a GAA protein (or a GAA protein comprising a sequence) at least 99%, at least 99.5%, or 100% identical to SEQ ID NO: 173. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 212 and encodes a GAA protein comprising the sequence set forth in SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 212. In another example, the GAA coding sequence is (or comprises a sequence) at least 95%, at least 96%, at least 97%, at least 98%, at least 99%, at least 99.5%, or 100% identical to SEQ ID NO: 212 and encodes a GAA protein (or a GAA protein comprising a sequence) at least 99%, at least 99.5%, or 100% identical to SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 212 and encodes a GAA protein comprising the sequence set forth in SEQ ID NO: 173. In another example, the GAA coding sequence is (or comprises a sequence) at least 99%, at least 99.5%, or 100% identical to SEQ ID NO: 212. In another example, the GAA coding sequence is (or comprises a sequence) at least 99%, at least 99.5%, or 100% identical to SEQ ID NO: 212 and encodes a GAA protein (or a GAA protein comprising a sequence) at least 99%, at least 99.5%, or 100% identical to SEQ ID NO: 173. In another example, the GAA coding sequence is (or comprises a sequence) at least 99%, at least 99.5%, or 100% identical to SEQ ID NO: 212 and encodes a GAA protein comprising the sequence set forth in SEQ ID NO: 173. In another example, the GAA coding sequence comprises the sequence set forth in SEQ ID NO: 212. In another example, the GAA coding sequence consists essentially of the sequence set forth in SEQ ID NO: 212. In another example, the GAA coding sequence consists of the sequence set forth in SEQ ID NO: 212. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

When specific GAA or multidomain therapeutic protein nucleic acid constructs sequences are disclosed herein, they are meant to encompass the sequence disclosed or the reverse complement of the sequence. For example, if a GAA or multidomain therapeutic protein nucleic acid construct disclosed herein consists of the hypothetical sequence 5′-CTGGACCGA-3′, it is also meant to encompass the reverse complement of that sequence (5′-TCGGTCCAG-3′). Likewise, when construct elements are disclosed herein in a specific 5′ to 3′ order, they are also meant to encompass the reverse complement of the order of those elements. One reason for this is that, in many embodiments disclosed herein, the GAA or multidomain therapeutic protein nucleic acid constructs are part of a single-stranded recombinant AAV vector. Single-stranded AAV genomes are packaged as either sense (plus-stranded) or anti-sense (minus-stranded genomes), and single-stranded AAV genomes of + and - polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

### (B) CD63-Binding Delivery Domain

The multidomain therapeutic proteins disclosed herein can comprise a CD63-binding delivery domain fused to a GAA. The CD63-binding domain provides binding to the internalization factor CD63 (UniProt Ref. P08962-1). CD63 (also known as CD63 antigen, granulophysin, lysosomal-associated membrane protein 3, LAMP-3, lysosome integral membrane protein 1, Limp1, melanoma-associated antigen ME491, OMA81H, ocular melanoma-associated antigen, tetraspanin-30, or Tspan-30) is a member of the tetraspanin superfamily of cell surface proteins that span the cell membrane four times. It is encoded by the CD63 gene (also known as MLA1 or TSPAN30). CD63 is expressed in virtually all tissues and is thought to be involved in forming and stabilizing signaling complexes. CD63 localizes to the cell membrane, lysosomal membrane, and late endosomal membrane. CD63 is known to associate with integrins and may be involved in epithelial-mesenchymal transitioning.

In some multidomain therapeutic proteins, the CD63-binding delivery domain is an antibody, an antibody fragment or other antigen-binding protein. In some multidomain therapeutic proteins, the CD63-binding delivery domain is an antigen-binding protein. Examples of antigen-binding proteins include, for example, a receptor-fusion molecule, a trap molecule, a receptor-Fc fusion molecule, an antibody, an Fab fragment, an F(ab′)2 fragment, an Fd fragment, an Fv fragment, a single-chain Fv (scFv) molecule, a dAb fragment, an isolated complementarity determining region (CDR), a CDR3 peptide, a constrained FR3-CDR3-FR4 peptide, a domain-specific antibody, a single domain antibody, a domain-deleted antibody, a chimeric antibody, a CDR-grafted antibody, a diabody, a triabody, a tetrabody, a minibody, a nanobody, a monovalent nanobody, a bivalent nanobody, a small modular immunopharmaceutical (SMIP), a camelid antibody (VHH heavy chain homodimeric antibody), and a shark variable IgNAR domain. Examples of CD63-binding delivery domains can be found in WO 2013/138400, WO 2017/007796, WO 2017/190079, WO 2017/100467, WO 2018/226861, WO 2019/157224, and WO 2019/222663, each of which is herein incorporated by reference in its entirety for all purposes.

In a particular multidomain therapeutic protein, the CD63-binding delivery domain is an anti-CD63 scFv. In a specific example, the anti-CD63 scFv can comprise SEQ ID NO: 183 or can be at least 90%, at least 91%, at least 92%, at least 93%, at least 94%, at least 95%, at least 96%, at least 97%, at least 98%, at least 99%, or at least 99.5% identical to SEQ ID NO: 183. In another specific example, the anti-CD63 scFv can consist essentially of SEQ ID NO: 183. In another specific example, the anti-CD63 scFv can consist of SEQ ID NO: 183.

The CD63-binding delivery domain coding sequences in the constructs disclosed herein may include one or more modifications such as codon optimization (e.g., to human codons), depletion of CpG dinucleotides, mutation of cryptic splice sites, addition of one or more glycosylation sites, or any combination thereof. CpG dinucleotides in a construct can limit the therapeutic utility of the construct. First, unmethylated CpG dinucleotides can interact with host toll-like receptor-9 (TLR-9) to stimulate innate, proinflammatory immune responses. Second, once the CpG dinucleotides become methylated, they can result in the suppression of transgene expression coordinated by methyl-CpG binding proteins. Cryptic splice sites are sequences in a pre-messenger RNA that are not normally used as splice sites, but that can be activated, for example, by mutations that either inactivate canonical splice sites or create splice sites where one did not exist before. Accurate splice site selection is critical for successful gene expression, and removal of cryptic splice sites can favor use of the normal or intended splice site.

In one example, a CD63-binding delivery domain coding sequence in a construct disclosed herein has one or more cryptic splice sites mutated or removed. In another example, a CD63-binding delivery domain coding sequence in a construct disclosed herein has all identified cryptic splice sites mutated or removed. In another example, a CD63-binding delivery domain coding sequence in a construct disclosed herein has one or more CpG dinucleotides removed (i.e., is CpG depleted). In another example, a CD63-binding delivery domain coding sequence in a construct disclosed herein has all CpG dinucleotides removed (i.e., is fully CpG depleted). In another example, a CD63-binding delivery domain coding sequence in a construct disclosed herein is codon optimized (e.g., codon optimized for expression in a human or mammal). In a specific example, a CD63-binding delivery domain coding sequence in a construct disclosed herein has one or more CpG dinucleotides removed (i.e., is CpG depleted) and has one or more cryptic splice sites mutated or removed. In another specific example, a CD63-binding delivery domain coding sequence in a construct disclosed herein has all CpG dinucleotides removed and has one or more or all identified cryptic splice sites mutated or removed. In another specific example, a CD63-binding delivery domain coding sequence in a construct disclosed herein has one or more CpG dinucleotides removed (i.e., is CpG depleted) and is codon optimized (e.g., codon optimized for expression in a human or mammal). In another specific example, a CD63-binding delivery domain coding sequence in a construct disclosed herein has all CpG dinucleotides removed (i.e., is fully CpG depleted) and is codon optimized (e.g., codon optimized for expression in a human or mammal).

Various anti-CD63 scFv coding sequences are provided. In one example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 184-192. In another example, the anti-CD63 scFv coding sequence is (or comprises a sequence) at least 95%, at least 96%, at least 97%, at least 98%, at least 99%, at least 99.5%, or 100% identical to any one of SEQ ID NOS: 184-192. In another example, the anti-CD63 scFv coding sequence is (or comprises a sequence) at least 99%, at least 99.5%, or 100% identical to any one of SEQ ID NOS: 184-192. In another example, the anti-CD63 scFv coding sequence comprises the sequence set forth in any one of SEQ ID NOS: 184-192. In another example, the anti-CD63 scFv coding sequence consists essentially of the sequence set forth in any one of SEQ ID NOS: 184-192. In another example, the anti-CD63 scFv coding sequence consists of the sequence set forth in any one of SEQ ID NOS: 184-192. In one example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 186. In another example, the anti-CD63 scFv coding sequence is (or comprises a sequence) at least 95%, at least 96%, at least 97%, at least 98%, at least 99%, at least 99.5%, or 100% identical to SEQ ID NO: 186. In another example, the anti-CD63 scFv coding sequence is (or comprises a sequence) at least 99%, at least 99.5%, or 100% identical to SEQ ID NO: 186. In another example, the anti-CD63 scFv coding sequence comprises the sequence set forth in SEQ ID NO: 186. In another example, the anti-CD63 scFv coding sequence consists essentially of the sequence set forth in SEQ ID NO: 186. In another example, the anti-CD63 scFv coding sequence consists of the sequence set forth in SEQ ID NO: 186. Optionally, the anti-CD63 scFv coding sequence encodes an anti-CD63 scFv protein (or an anti-CD63 scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 183 (and, e.g., retaining CD63-binding activity). Optionally, the anti-CD63 scFv coding sequence encodes an anti-CD63 scFv protein (or an anti-CD63 scFv protein comprising a sequence) at least 95%, at least 96%, at least 97%, at least 98%, at least 99%, at least 99.5%, or 100% identical to SEQ ID NO: 183 (and, e.g., retaining CD63-binding activity). Optionally, the anti-CD63 scFv coding sequence in the above examples encodes an anti-CD63 scFv protein (or an anti-CD63 scFv protein comprising a sequence) at least 99%, at least 99.5%, or 100% identical to SEQ ID NO: 183 (and, e.g., retaining CD63-binding activity). Optionally, the anti-CD63 scFv coding sequence in the above examples encodes an anti-CD63 scFv protein comprising the sequence set forth in SEQ ID NO: 183. Optionally, the anti-CD63 scFv coding sequence in the above examples encodes an anti-CD63 scFv protein consisting essentially of the sequence set forth in SEQ ID NO: 183. Optionally, the anti-CD63 scFv coding sequence in the above examples encodes an anti-CD63 scFv protein consisting of the sequence set forth in SEQ ID NO: 183.

Various codon optimized anti-CD63 scFv coding sequences are provided. The anti-CD63 scFv coding sequence can be, for example, CpG-depleted (e.g., fully CpG depleted) and/or codon optimized (e.g., CpG depleted (e.g., fully CpG-depleted) and codon optimized). In one example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 185-192. In another example, the anti-CD63 scFv coding sequence is (or comprises a sequence) at least 95%, at least 96%, at least 97%, at least 98%, at least 99%, at least 99.5%, or 100% identical to any one of SEQ ID NOS: 185-192. In another example, the anti-CD63 scFv coding sequence is (or comprises a sequence) at least 99%, at least 99.5%, or 100% identical to any one of SEQ ID NOS: 185-192. In another example, the anti-CD63 scFv coding sequence comprises the sequence set forth in any one of SEQ ID NOS: 185-192. In another example, the anti-CD63 scFv coding sequence consists essentially of the sequence set forth in any one of SEQ ID NOS: 185-192. In another example, the anti-CD63 scFv coding sequence consists of the sequence set forth in any one of SEQ ID NOS: 185-192. Optionally, the anti-CD63 scFv coding sequence encodes an anti-CD63 scFv protein (or an anti-CD63 scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 183 (and, e.g., retaining CD63-binding activity). Optionally, the anti-CD63 scFv coding sequence encodes an anti-CD63 scFv protein (or an anti-CD63 scFv protein comprising a sequence) at least 95%, at least 96%, at least 97%, at least 98%, at least 99%, at least 99.5%, or 100% identical to SEQ ID NO: 183 (and, e.g., retaining CD63-binding activity). Optionally, the anti-CD63 scFv coding sequence in the above examples encodes an anti-CD63 scFv protein (or an anti-CD63 scFv protein comprising a sequence) at least 99%, at least 99.5%, or 100% identical to SEQ ID NO: 183 (and, e.g., retaining CD63-binding activity). Optionally, the anti-CD63 scFv coding sequence in the above examples encodes an anti-CD63 scFv protein comprising the sequence set forth in SEQ ID NO: 183. Optionally, the anti-CD63 scFv coding sequence in the above examples encodes an anti-CD63 scFv protein consisting essentially of the sequence set forth in SEQ ID NO: 183. Optionally, the anti-CD63 scFv coding sequence in the above examples encodes an anti-CD63 scFv protein consisting of the sequence set forth in SEQ ID NO: 183.

In one example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 186. In another example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 186 and encodes an anti-CD63 scFv protein (or an anti-CD63 scFv protein comprising a sequence) at least 99%, at least 99.5%, or 100% identical to SEQ ID NO: 183. In another example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 186 and encodes an anti-CD63 scFv protein comprising the sequence set forth in SEQ ID NO: 183. In another example, the anti-CD63 scFv coding sequence is (or comprises a sequence) at least 95%, at least 96%, at least 97%, at least 98%, at least 99%, at least 99.5%, or 100% identical to SEQ ID NO: 186. In another example, the anti-CD63 scFv coding sequence is (or comprises a sequence) at least 95%, at least 96%, at least 97%, at least 98%, at least 99%, at least 99.5%, or 100% identical to SEQ ID NO: 186 and encodes an anti-CD63 scFv protein (or an anti-CD63 scFv protein comprising a sequence) at least 99%, at least 99.5%, or 100% identical to SEQ ID NO: 183. In another example, the anti-CD63 scFv coding sequence is (or comprises a sequence) at least 95%, at least 96%, at least 97%, at least 98%, at least 99%, at least 99.5%, or 100% identical to SEQ ID NO: 186 and encodes an anti-CD63 scFv protein comprising the sequence set forth in SEQ ID NO: 183. In another example, the anti-CD63 scFv coding sequence is (or comprises a sequence) at least 99%, at least 99.5%, or 100% identical to SEQ ID NO: 186. In another example, the anti-CD63 scFv coding sequence is (or comprises a sequence) at least 99%, at least 99.5%, or 100% identical to SEQ ID NO: 186 and encodes an anti-CD63 scFv protein (or an anti-CD63 scFv protein comprising a sequence) at least 99%, at least 99.5%, or 100% identical to SEQ ID NO: 183. In another example, the anti-CD63 scFv coding sequence is (or comprises a sequence) at least 99%, at least 99.5%, or 100% identical to SEQ ID NO: 186 and encodes an anti-CD63 scFv protein comprising the sequence set forth in SEQ ID NO: 183. In another example, the anti-CD63 scFv coding sequence comprises the sequence set forth in SEQ ID NO: 186. In another example, the anti-CD63 scFv coding sequence consists essentially of the sequence set forth in SEQ ID NO: 186. In another example, the anti-CD63 scFv coding sequence consists of the sequence set forth in SEQ ID NO: 186. The anti-CD63 scFv coding sequence can be, for example, CpG-depleted (e.g., fully CpG-depleted) and/or codon optimized. For example, the anti-CD63 scFv coding sequence can be CpG depleted (e.g., fully CpG-depleted) and codon optimized. Optionally, the anti-CD63 scFv coding sequence encodes an anti-CD63 scFv protein (or an anti-CD63 scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 183 (and, e.g., retaining CD63-binding activity). Optionally, the anti-CD63 scFv coding sequence encodes an anti-CD63 scFv protein (or an anti-CD63 scFv protein comprising a sequence) at least 95%, at least 96%, at least 97%, at least 98%, at least 99%, at least 99.5%, or 100% identical to SEQ ID NO: 183 (and, e.g., retaining CD63-binding activity). Optionally, the anti-CD63 scFv coding sequence in the above examples encodes an anti-CD63 scFv protein (or an anti-CD63 scFv protein comprising a sequence) at least 99%, at least 99.5%, or 100% identical to SEQ ID NO: 183 (and, e.g., retaining CD63-binding activity). Optionally, the anti-CD63 scFv coding sequence in the above examples encodes an anti-CD63 scFv protein comprising the sequence set forth in SEQ ID NO: 183. Optionally, the anti-CD63 scFv coding sequence in the above examples encodes an anti-CD63 scFv protein consisting essentially of the sequence set forth in SEQ ID NO: 183. Optionally, the anti-CD63 scFv coding sequence in the above examples encodes an anti-CD63 scFv protein consisting of the sequence set forth in SEQ ID NO: 183.

In one example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 184. In another example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 184 and encodes an anti-CD63 scFv protein (or an anti-CD63 scFv protein comprising a sequence) at least 99%, at least 99.5%, or 100% identical to SEQ ID NO: 183. In another example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 184 and encodes an anti-CD63 scFv protein comprising the sequence set forth in SEQ ID NO: 183. In another example, the anti-CD63 scFv coding sequence is (or comprises a sequence) at least 95%, at least 96%, at least 97%, at least 98%, at least 99%, at least 99.5%, or 100% identical to SEQ ID NO: 184. In another example, the anti-CD63 scFv coding sequence is (or comprises a sequence) at least 95%, at least 96%, at least 97%, at least 98%, at least 99%, at least 99.5%, or 100% identical to SEQ ID NO: 184 and encodes an anti-CD63 scFv protein (or an anti-CD63 scFv protein comprising a sequence) at least 99%, at least 99.5%, or 100% identical to SEQ ID NO: 183. In another example, the anti-CD63 scFv coding sequence is (or comprises a sequence) at least 95%, at least 96%, at least 97%, at least 98%, at least 99%, at least 99.5%, or 100% identical to SEQ ID NO: 184 and encodes an anti-CD63 scFv protein comprising the sequence set forth in SEQ ID NO: 183. In another example, the anti-CD63 scFv coding sequence is (or comprises a sequence) at least 99%, at least 99.5%, or 100% identical to SEQ ID NO: 184. In another example, the anti-CD63 scFv coding sequence is (or comprises a sequence) at least 99%, at least 99.5%, or 100% identical to SEQ ID NO: 184 and encodes an anti-CD63 scFv protein (or an anti-CD63 scFv protein comprising a sequence) at least 99%, at least 99.5%, or 100% identical to SEQ ID NO: 183. In another example, the anti-CD63 scFv coding sequence is (or comprises a sequence) at least 99%, at least 99.5%, or 100% identical to SEQ ID NO: 184 and encodes an anti-CD63 scFv protein comprising the sequence set forth in SEQ ID NO: 183. In another example, the anti-CD63 scFv coding sequence comprises the sequence set forth in SEQ ID NO: 184. In another example, the anti-CD63 scFv coding sequence consists essentially of the sequence set forth in SEQ ID NO: 184. In another example, the anti-CD63 scFv coding sequence consists of the sequence set forth in SEQ ID NO: 184. The anti-CD63 scFv coding sequence can be, for example, CpG-depleted (e.g., fully CpG-depleted) and/or codon optimized. For example, the anti-CD63 scFv coding sequence can be CpG depleted (e.g., fully CpG-depleted) and codon optimized. Optionally, the anti-CD63 scFv coding sequence encodes an anti-CD63 scFv protein (or an anti-CD63 scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 183 (and, e.g., retaining CD63-binding activity). Optionally, the anti-CD63 scFv coding sequence encodes an anti-CD63 scFv protein (or an anti-CD63 scFv protein comprising a sequence) at least 95%, at least 96%, at least 97%, at least 98%, at least 99%, at least 99.5%, or 100% identical to SEQ ID NO: 183 (and, e.g., retaining CD63-binding activity). Optionally, the anti-CD63 scFv coding sequence in the above examples encodes an anti-CD63 scFv protein (or an anti-CD63 scFv protein comprising a sequence) at least 99%, at least 99.5%, or 100% identical to SEQ ID NO: 183 (and, e.g., retaining CD63-binding activity). Optionally, the anti-CD63 scFv coding sequence in the above examples encodes an anti-CD63 scFv protein comprising the sequence set forth in SEQ ID NO: 183. Optionally, the anti-CD63 scFv coding sequence in the above examples encodes an anti-CD63 scFv protein consisting essentially of the sequence set forth in SEQ ID NO: 183. Optionally, the anti-CD63 scFv coding sequence in the above examples encodes an anti-CD63 scFv protein consisting of the sequence set forth in SEQ ID NO: 183.

In one example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 191. In another example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 191 and encodes an anti-CD63 scFv protein (or an anti-CD63 scFv protein comprising a sequence) at least 99%, at least 99.5%, or 100% identical to SEQ ID NO: 183. In another example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 191 and encodes an anti-CD63 scFv protein comprising the sequence set forth in SEQ ID NO: 183. In another example, the anti-CD63 scFv coding sequence is (or comprises a sequence) at least 95%, at least 96%, at least 97%, at least 98%, at least 99%, at least 99.5%, or 100% identical to SEQ ID NO: 191. In another example, the anti-CD63 scFv coding sequence is (or comprises a sequence) at least 95%, at least 96%, at least 97%, at least 98%, at least 99%, at least 99.5%, or 100% identical to SEQ ID NO: 191 and encodes an anti-CD63 scFv protein (or an anti-CD63 scFv protein comprising a sequence) at least 99%, at least 99.5%, or 100% identical to SEQ ID NO: 183. In another example, the anti-CD63 scFv coding sequence is (or comprises a sequence) at least 95%, at least 96%, at least 97%, at least 98%, at least 99%, at least 99.5%, or 100% identical to SEQ ID NO: 191 and encodes an anti-CD63 scFv protein comprising the sequence set forth in SEQ ID NO: 183. In another example, the anti-CD63 scFv coding sequence is (or comprises a sequence) at least 99%, at least 99.5%, or 100% identical to SEQ ID NO: 191. In another example, the anti-CD63 scFv coding sequence is (or comprises a sequence) at least 99%, at least 99.5%, or 100% identical to SEQ ID NO: 191 and encodes an anti-CD63 scFv protein (or an anti-CD63 scFv protein comprising a sequence) at least 99%, at least 99.5%, or 100% identical to SEQ ID NO: 183. In another example, the anti-CD63 scFv coding sequence is (or comprises a sequence) at least 99%, at least 99.5%, or 100% identical to SEQ ID NO: 191 and encodes an anti-CD63 scFv protein comprising the sequence set forth in SEQ ID NO: 183. In another example, the anti-CD63 scFv coding sequence comprises the sequence set forth in SEQ ID NO: 191. In another example, the anti-CD63 scFv coding sequence consists essentially of the sequence set forth in SEQ ID NO: 191. In another example, the anti-CD63 scFv coding sequence consists of the sequence set forth in SEQ ID NO: 191. The anti-CD63 scFv coding sequence can be, for example, CpG-depleted (e.g., fully CpG-depleted) and/or codon optimized. For example, the anti-CD63 scFv coding sequence can be CpG depleted (e.g., fully CpG-depleted) and codon optimized. Optionally, the anti-CD63 scFv coding sequence encodes an anti-CD63 scFv protein (or an anti-CD63 scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 183 (and, e.g., retaining CD63-binding activity). Optionally, the anti-CD63 scFv coding sequence encodes an anti-CD63 scFv protein (or an anti-CD63 scFv protein comprising a sequence) at least 95%, at least 96%, at least 97%, at least 98%, at least 99%, at least 99.5%, or 100% identical to SEQ ID NO: 183 (and, e.g., retaining CD63-binding activity). Optionally, the anti-CD63 scFv coding sequence in the above examples encodes an anti-CD63 scFv protein (or an anti-CD63 scFv protein comprising a sequence) at least 99%, at least 99.5%, or 100% identical to SEQ ID NO: 183 (and, e.g., retaining CD63-binding activity). Optionally, the anti-CD63 scFv coding sequence in the above examples encodes an anti-CD63 scFv protein comprising the sequence set forth in SEQ ID NO: 183. Optionally, the anti-CD63 scFv coding sequence in the above examples encodes an anti-CD63 scFv protein consisting essentially of the sequence set forth in SEQ ID NO: 183. Optionally, the anti-CD63 scFv coding sequence in the above examples encodes an anti-CD63 scFv protein consisting of the sequence set forth in SEQ ID NO: 183.

In one example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 190. In another example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 190 and encodes an anti-CD63 scFv protein (or an anti-CD63 scFv protein comprising a sequence) at least 99%, at least 99.5%, or 100% identical to SEQ ID NO: 183. In another example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 190 and encodes an anti-CD63 scFv protein comprising the sequence set forth in SEQ ID NO: 183. In another example, the anti-CD63 scFv coding sequence is (or comprises a sequence) at least 95%, at least 96%, at least 97%, at least 98%, at least 99%, at least 99.5%, or 100% identical to SEQ ID NO: 190. In another example, the anti-CD63 scFv coding sequence is (or comprises a sequence) at least 95%, at least 96%, at least 97%, at least 98%, at least 99%, at least 99.5%, or 100% identical to SEQ ID NO: 190 and encodes an anti-CD63 scFv protein (or an anti-CD63 scFv protein comprising a sequence) at least 99%, at least 99.5%, or 100% identical to SEQ ID NO: 183. In another example, the anti-CD63 scFv coding sequence is (or comprises a sequence) at least 95%, at least 96%, at least 97%, at least 98%, at least 99%, at least 99.5%, or 100% identical to SEQ ID NO: 190 and encodes an anti-CD63 scFv protein comprising the sequence set forth in SEQ ID NO: 183. In another example, the anti-CD63 scFv coding sequence is (or comprises a sequence) at least 99%, at least 99.5%, or 100% identical to SEQ ID NO: 190. In another example, the anti-CD63 scFv coding sequence is (or comprises a sequence) at least 99%, at least 99.5%, or 100% identical to SEQ ID NO: 190 and encodes an anti-CD63 scFv protein (or an anti-CD63 scFv protein comprising a sequence) at least 99%, at least 99.5%, or 100% identical to SEQ ID NO: 183. In another example, the anti-CD63 scFv coding sequence is (or comprises a sequence) at least 99%, at least 99.5%, or 100% identical to SEQ ID NO: 190 and encodes an anti-CD63 scFv protein comprising the sequence set forth in SEQ ID NO: 183. In another example, the anti-CD63 scFv coding sequence comprises the sequence set forth in SEQ ID NO: 190. In another example, the anti-CD63 scFv coding sequence consists essentially of the sequence set forth in SEQ ID NO: 190. In another example, the anti-CD63 scFv coding sequence consists of the sequence set forth in SEQ ID NO: 190. The anti-CD63 scFv coding sequence can be, for example, CpG-depleted (e.g., fully CpG-depleted) and/or codon optimized. For example, the anti-CD63 scFv coding sequence can be CpG depleted (e.g., fully CpG-depleted) and codon optimized. Optionally, the anti-CD63 scFv coding sequence encodes an anti-CD63 scFv protein (or an anti-CD63 scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 183 (and, e.g., retaining CD63-binding activity). Optionally, the anti-CD63 scFv coding sequence encodes an anti-CD63 scFv protein (or an anti-CD63 scFv protein comprising a sequence) at least 95%, at least 96%, at least 97%, at least 98%, at least 99%, at least 99.5%, or 100% identical to SEQ ID NO: 183 (and, e.g., retaining CD63-binding activity). Optionally, the anti-CD63 scFv coding sequence in the above examples encodes an anti-CD63 scFv protein (or an anti-CD63 scFv protein comprising a sequence) at least 99%, at least 99.5%, or 100% identical to SEQ ID NO: 183 (and, e.g., retaining CD63-binding activity). Optionally, the anti-CD63 scFv coding sequence in the above examples encodes an anti-CD63 scFv protein comprising the sequence set forth in SEQ ID NO: 183. Optionally, the anti-CD63 scFv coding sequence in the above examples encodes an anti-CD63 scFv protein consisting essentially of the sequence set forth in SEQ ID NO: 183. Optionally, the anti-CD63 scFv coding sequence in the above examples encodes an anti-CD63 scFv protein consisting of the sequence set forth in SEQ ID NO: 183.

In one example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 188. In another example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 188 and encodes an anti-CD63 scFv protein (or an anti-CD63 scFv protein comprising a sequence) at least 99%, at least 99.5%, or 100% identical to SEQ ID NO: 183. In another example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 188 and encodes an anti-CD63 scFv protein comprising the sequence set forth in SEQ ID NO: 183. In another example, the anti-CD63 scFv coding sequence is (or comprises a sequence) at least 95%, at least 96%, at least 97%, at least 98%, at least 99%, at least 99.5%, or 100% identical to SEQ ID NO: 188. In another example, the anti-CD63 scFv coding sequence is (or comprises a sequence) at least 95%, at least 96%, at least 97%, at least 98%, at least 99%, at least 99.5%, or 100% identical to SEQ ID NO: 188 and encodes an anti-CD63 scFv protein (or an anti-CD63 scFv protein comprising a sequence) at least 99%, at least 99.5%, or 100% identical to SEQ ID NO: 183. In another example, the anti-CD63 scFv coding sequence is (or comprises a sequence) at least 95%, at least 96%, at least 97%, at least 98%, at least 99%, at least 99.5%, or 100% identical to SEQ ID NO: 188 and encodes an anti-CD63 scFv protein comprising the sequence set forth in SEQ ID NO: 183. In another example, the anti-CD63 scFv coding sequence is (or comprises a sequence) at least 99%, at least 99.5%, or 100% identical to SEQ ID NO: 188. In another example, the anti-CD63 scFv coding sequence is (or comprises a sequence) at least 99%, at least 99.5%, or 100% identical to SEQ ID NO: 188 and encodes an anti-CD63 scFv protein (or an anti-CD63 scFv protein comprising a sequence) at least 99%, at least 99.5%, or 100% identical to SEQ ID NO: 183. In another example, the anti-CD63 scFv coding sequence is (or comprises a sequence) at least 99%, at least 99.5%, or 100% identical to SEQ ID NO: 188 and encodes an anti-CD63 scFv protein comprising the sequence set forth in SEQ ID NO: 183. In another example, the anti-CD63 scFv coding sequence comprises the sequence set forth in SEQ ID NO: 188. In another example, the anti-CD63 scFv coding sequence consists essentially of the sequence set forth in SEQ ID NO: 188. In another example, the anti-CD63 scFv coding sequence consists of the sequence set forth in SEQ ID NO: 188. The anti-CD63 scFv coding sequence can be, for example, CpG-depleted (e.g., fully CpG-depleted) and/or codon optimized. For example, the anti-CD63 scFv coding sequence can be CpG depleted (e.g., fully CpG-depleted) and codon optimized. Optionally, the anti-CD63 scFv coding sequence encodes an anti-CD63 scFv protein (or an anti-CD63 scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 183 (and, e.g., retaining CD63-binding activity). Optionally, the anti-CD63 scFv coding sequence encodes an anti-CD63 scFv protein (or an anti-CD63 scFv protein comprising a sequence) at least 95%, at least 96%, at least 97%, at least 98%, at least 99%, at least 99.5%, or 100% identical to SEQ ID NO: 183 (and, e.g., retaining CD63-binding activity). Optionally, the anti-CD63 scFv coding sequence in the above examples encodes an anti-CD63 scFv protein (or an anti-CD63 scFv protein comprising a sequence) at least 99%, at least 99.5%, or 100% identical to SEQ ID NO: 183 (and, e.g., retaining CD63-binding activity). Optionally, the anti-CD63 scFv coding sequence in the above examples encodes an anti-CD63 scFv protein comprising the sequence set forth in SEQ ID NO: 183. Optionally, the anti-CD63 scFv coding sequence in the above examples encodes an anti-CD63 scFv protein consisting essentially of the sequence set forth in SEQ ID NO: 183. Optionally, the anti-CD63 scFv coding sequence in the above examples encodes an anti-CD63 scFv protein consisting of the sequence set forth in SEQ ID NO: 183.

When specific anti-CD63 scFv or multidomain therapeutic protein nucleic acid constructs sequences are disclosed herein, they are meant to encompass the sequence disclosed or the reverse complement of the sequence. For example, if an anti-CD63 scFv or multidomain therapeutic protein nucleic acid construct disclosed herein consists of the hypothetical sequence 5′-CTGGACCGA-3′, it is also meant to encompass the reverse complement of that sequence (5′-TCGGTCCAG-3′). Likewise, when construct elements are disclosed herein in a specific 5′ to 3′ order, they are also meant to encompass the reverse complement of the order of those elements. One reason for this is that, in many embodiments disclosed herein, the anti-CD63 scFv or multidomain therapeutic protein nucleic acid constructs are part of a single-stranded recombinant AAV vector. Single-stranded AAV genomes are packaged as either sense (plus-stranded) or anti-sense (minus-stranded genomes), and single-stranded AAV genomes of + and -polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

### (C) TfR-Binding Delivery Domain

The multidomain therapeutic proteins disclosed herein can comprise a TfR-binding delivery domain fused to a GAA. The TfR-binding domain provides binding to the internalization factor transferrin receptor protein 1(TfR; UniProt Ref. P02786). TfR (also known as TR, TfR1, and Trfr) is encoded by the TFRC gene. TfR is expressed in muscle and on brain endothelial cells. Transcytosis of TfR in these cells enables blood-brain-barrier crossing. In some embodiments, the multidomain therapeutic proteins comprising a TfR-binding delivery domain (e.g., scFv) fused to a GAA do not alter transferrin uptake. In some embodiments, the multidomain therapeutic proteins comprising a TfR-binding delivery domain (e.g., scFv) fused to a GAA do not alter iron homeostasis. In some embodiments, the multidomain therapeutic proteins comprising a TfR-binding delivery domain (e.g., scFv) fused to a GAA do not alter transferrin uptake or iron homeostasis.

Transferrin receptor 1 (TfR) is a membrane receptor involved in the control of iron supply to the cell through the binding of transferrin, the major iron-carrier protein. Transferrin receptor 1 is expressed from the TFRC gene. Transferrin receptor 1 may be referred to, herein, at TFRC. This receptor plays a key role in the control of cell proliferation because iron is essential for sustaining ribonucleotide reductase activity, and is the only enzyme that catalyzes the conversion of ribonucleotides to deoxyribonucleotides. Preferably, the TfR is human TfR (hTfR). See e.g.., Accession numbers NP_ 001121620.1; BAD92491.1; and NP_001300894.1.; and e!Ensembl entry: ENSG00000072274. The human transferrin receptor 1 is expressed in several tissues, including but not limited to: cerebral cortex; cerebellum; hippocampus; caudate; parathyroid gland; adrenal gland; bronchus; lung; oral mucosa; esophagus; stomach; duodenum; small intestine; colon; rectum; liver; gallbladder; pancreas; kidney; urinary bladder; testis; epididymis; prostate; vagina; ovary; fallopian tube; endometrium; cervix; placenta; breast; heart muscle; smooth muscle; soft tissue; skin; appendix; lymph node; tonsil; and bone marrow. A related transferrin receptor is transferrin receptor 2 (TfR2). Human transferrin receptor 2 bears about 45% sequence identity to human transferrin receptor 1. Trinder & Baker, Transferrin receptor 2: a new molecule in iron metabolism. Int J Biochem Cell Biol. 2003 Mar;35(3):292-6. Unless otherwise stated, transferrin receptor as used herein generally refers to transferrin receptor 1 (e.g., human transferrin receptor 1).

Human Transferrin (Tf) is a single chain, 80 kDa member of the anion-binding superfamily of proteins. Transferrin is a 698 amino acid precursor that is divided into a 19 aa signal sequence plus a 679 aa mature segment that typically contains 19 intrachain disulfide bonds. The N- and C-terminal flanking regions (or domains) bind ferric iron through the interaction of an obligate anion (e.g., bicarbonate) and four amino acids (His, Asp, and two Tyr). Apotransferrin (or iron-free) will initially bind one atom of iron at the C-terminus, and this is followed by subsequent iron binding by the N-terminus to form holotransferrin (diferric Tf, Holo-Tf). Through its C-terminal iron-binding domain, holotransferrin will interact with the TfR on the surface of cells where it is internalized into acidified endosomes. Iron dissociates from the Tf molecule within these endosomes, and is transported into the cytosol as ferrous iron. In addition to TfR, transferrin is reported to bind to cubulin, IGFBP3, microbial iron-binding proteins and liver-specific TfR2.

The blood-brain barrier (BBB) is located within the microvasculature of the brain, and it regulates passage of molecules from the blood to the brain. Burkhart et al., Accessing targeted nanoparticles to the brain: the vascular route. Curr Med Chem. 2014;21(36):4092-9. The transcellular passage through the brain capillary endothelial cells can take place via 1) cell entry by leukocytes; 2) carrier-mediated influx of e.g., glucose by glucose transporter 1 (GLUT-1), amino acids by e.g., the L- type amino acid transporter 1 (LAT-1) and small peptides by e.g., organic anion-transporting peptide-B (OATP-B); 3) paracellular passage of small hydrophobic molecules; 4) adsorption-mediated transcytosis of e.g., albumin and cationized molecules; 5) passive diffusion of lipid soluble, non-polar solutes, including CO₂ and O₂; and 5) receptor-mediated transcytosis of, e.g., insulin by the insulin receptor and Tf by the TfR. Johnsen et al., Targeting the transferrin receptor for brain drug delivery, Prog Neurobiol. 2019 Oct;181:101665.

For example, anti-TfR:GAA fusion proteins exhibiting high affinity to the transferrin receptor and superior blood-brain barrier crossing are provided. Surprisingly, fusions exhibiting high binding affinity to TfR crossed the blood-brain barrier more efficiently than that of low affinity binders. The fusions of the present invention have an ability to efficiently deliver GAA to the brain and, thus, are an effective treatment of glycogen storage diseases such as Pompe Disease.

Provided herein are antigen-binding proteins, such as antibodies, antigen-binding fragments thereof, such as Fabs and scFvs, that bind specifically to the transferrin receptor, preferably the human transferrin receptor 1 (anti-hTfR). For example, in an embodiment, the anti-hTfR is in the form of a fusion protein. The fusion protein includes the anti-hTfR antigen-binding protein fused to GAA. The anti-hTfRs efficiently cross the blood-brain barrier (BBB) and can, thereby, deliver the fused GAA to the brain.

An antigen-binding protein that specifically binds to transferrin receptor and fusions thereof, for example, a tag such as His₆ and/or myc (e.g., human transferrin receptor (e.g., REGN2431) or monkey transferrin receptor (e.g., REGN2054)) binds at about 25° C., e.g., in a surface plasmon resonance assay, with a K_{D} of about 20 nM or a higher affinity. Such an antigen-binding protein may be referred to as “anti-TfR.”

In an embodiment, an anti-hTfR scFv:GAA fusion protein includes an scFv comprising the arrangement of variable regions as follows LCVR-HCVR or HCVR-LCVR, wherein the HCVR and LCVR are optionally connected by a linker and the scFv is connected, optionally by a linker, to GAA (e.g., LCVR-(Gly₄Ser)₃-HCVR-(Gly₄Ser)₂)-GAA; or LCVR-(Gly₄Ser)₃-HCVR-(Gly₄Ser)₂)-GAA) (Gly₄Ser = SEQ ID NO: 600)). In one example, the linker between the HCVR and LCVR comprises, consists essentially of, or consists of three such repeats (SEQ ID NO: 713). For example, the coding sequence for the linker can comprise, consist essentially of, or consist of any one of SEQ ID NOS: 715-719. In another example, the linker between the HCVR and LCVR comprises, consists essentially of, or consists of two such repeats (SEQ ID NO: 714). For example, the coding sequence for the linker can comprise, consist essentially of, or consist of any one of SEQ ID NOS: 720-726. In another example, the linker between the HCVR and LCVR comprises, consists essentially of, or consists of one such repeat (SEQ ID NO: 600). For example, the coding sequence for the linker can comprise, consist essentially of, or consist of SEQ ID NO: 727. In one example, the linker between the scFv and GAA comprises, consists essentially of, or consists of three such repeats (SEQ ID NO: 713). For example, the coding sequence for the linker can comprise, consist essentially of, or consist of any one of SEQ ID NOS: 715-719. In another example, the linker between the scFv and GAA comprises, consists essentially of, or consists of two such repeats (SEQ ID NO: 714). For example, the coding sequence for the linker can comprise, consist essentially of, or consist of any one of SEQ ID NOS: 720-726. In another example, the linker between the scFv and GAA comprises, consists essentially of, or consists of one such repeat (SEQ ID NO: 600). For example, the coding sequence for the linker can comprise, consist essentially of, or consist of SEQ ID NO: 727.

An anti-hTfR:GAA optionally comprises a signal peptide, connected to the antigen-binding protein that binds specifically to transferrin receptor (TfR), preferably, human transferrin receptor (hTfR) which is fused (optionally by a linker) to GAA. In an embodiment, the signal peptide is the mROR signal sequence (e.g., mROR signal sequence-LCVR-(Gly₄Ser)₃-HCVR-(Gly₄Ser)₂)-GAA; or LCVR-(Gly₄Ser)₃-HCVR-(Gly₄Ser)₂)-GAA) (Gly₄Ser = SEQ ID NO: 600)). The term “fused” or “tethered” with regard to fused polypeptides refers to polypeptides joined directly or indirectly (e.g., via a linker or other polypeptide).

In an embodiment of the invention, the assignment of amino acids to each framework or CDR domain in an immunoglobulin is in accordance with the definitions of Sequences of Proteins of Immunological Interest, Kabat et al.; National Institutes of Health, Bethesda, Md.; 5^{th} ed.; NIH Publ. No. 91-3242 (1991); Kabat (1978) Adv. Prot. Chem. 32:1-75; Kabat et al., (1977) J. Biol. Chem. 252:6609-6616; Chothia, et al., (1987) J Mol. Biol. 196:901-917 or Chothia, et al., (1989) Nature 342: 878-883. Thus, included are antibodies and antigen-binding fragments including the CDRs of a V_{H} and the CDRs of a V_{L}, which V_{H} and V_{L} comprise amino acid sequences as set forth herein (see, e.g., sequences of Table 29, or variants thereof), wherein the CDRs are as defined according to Kabat and/or Chothia.

In some multidomain therapeutic proteins, the TfR-binding delivery domain is an antibody, an antibody fragment or other antigen-binding protein. In some multidomain therapeutic proteins, the TfR-binding delivery domain is an antigen-binding protein. Examples of antigen-binding proteins include, for example, a receptor-fusion molecule, a trap molecule, a receptor-Fc fusion molecule, an antibody, an Fab fragment, an F(ab′)2 fragment, an Fd fragment, an Fv fragment, a single-chain Fv (scFv) molecule, a dAb fragment, an isolated complementarity determining region (CDR), a CDR3 peptide, a constrained FR3-CDR3-FR4 peptide, a domain-specific antibody, a single domain antibody, a domain-deleted antibody, a chimeric antibody, a CDR-grafted antibody, a diabody, a triabody, a tetrabody, a minibody, a nanobody, a monovalent nanobody, a bivalent nanobody, a small modular immunopharmaceutical (SMIP), a camelid antibody (VHH heavy chain homodimeric antibody), and a shark variable IgNAR domain.

Provided herein are antibodies that bind specifically to the human transferrin receptor 1. The term “antibody,” as used herein, refers to immunoglobulin molecules comprising four polypeptide chains, two heavy chains (HCs) and two light chains (LCs), inter-connected by disulfide bonds. In an embodiment, each antibody heavy chain (HC) comprises a heavy chain variable region (“HCVR” or “V_{H}”) (e.g., comprising SEQ ID NO: 217, 227, 237, 247, 257, 267, 277, 287, 297, 307, 317, 327, 337, 347, 357, 367, 377, 387, 397, 407, 417, 427, 437, 447, 457, 467, 477, 487, 497, 507, 517, and/or 527 or a variant thereof) and a heavy chain constant region (e.g., human IgG, human IgG1 or human IgG4); and each antibody light chain (LC) comprises a light chain variable region (“LCVR or “V_{L}”) (e.g., SEQ ID NO: 222, 232, 242, 252, 262, 272, 282, 292, 302, 312, 322, 332, 342, 352, 362, 372, 382, 392, 402, 412, 422, 432, 442, 452, 462, 472, 482, 492, 502, 512, 522, and/or 532 or a variant thereof) and a light chain constant region (e.g., human kappa or human lambda). The V_{H} and V_{L} regions can be further subdivided into regions of hypervariability, termed complementarity determining regions (CDR), interspersed with regions that are more conserved, termed framework regions (FR) Each V_{H} and V_{L} comprises three CDRs and four FRs. Anti-TfR antibodies disclosed herein can also be fused to GAA.

An anti-TfR antigen-binding protein of the present invention may be an antigen-binding fragment of an antibody which may be tethered to GAA. The terms “antigen-binding portion” or “antigen-binding fragment” of an antibody, as used herein, refers to an immunoglobulin molecule that binds antigen but that does not include all of the sequences of a full antibody (preferably, the full antibody is an IgG). Non-limiting examples of antigen-binding fragments include: (i) Fab fragments; (ii) F(ab′)₂ fragments; (iii) Fd fragments; (iv) Fv fragments; (v) single-chain Fv (scFv) molecules; and (vi) dAb fragments; consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies and small modular immunopharmaceuticals (SMIPs), are also encompassed within the expression “antigen-binding fragment,” as used herein.

An anti-TfR antigen-binding protein may be an scFv which may be tethered to a GAA. An scFv (single chain fragment variable) has variable regions of heavy (V_{H}) and light (V_{L}) domains (in either order), which, preferably, are joined together by a flexible linker (e.g., peptide linker). The length of the flexible linker used to link both of the V regions may be important for yielding the correct folding of the polypeptide chain. Previously, it has been estimated that the peptide linker must span 3.5 nm (35 Å) between the carboxy terminus of the variable domain and the amino terminus of the other domain without affecting the ability of the domains to fold and form an intact antigen-binding site (Huston et al., Protein engineering of single-chain Fv analogs and fusion proteins. Methods in Enzymology. 1991;203:46-88). In an embodiment, the linker comprises an amino acid sequence of such length to separate the variable domains by about 3.5 nm.

An antigen-binding fragment of an antibody will, in an embodiment, comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H} - V_{H}, V_{H} - V_{L} or V_{L} - V_{L} dimers. Alternatively, the antigen-binding fragment of an antibody may contain a monomeric V_{H} or V_{L} domain.

“Isolated” antigen-binding proteins (e.g., antibodies or antigen-binding fragments thereof), polypeptides, polynucleotides and vectors, are at least partially free of other biological molecules from the cells or cell culture from which they are produced. Such biological molecules include nucleic acids, proteins, other antibodies or antigen-binding fragments, lipids, carbohydrates, or other material such as cellular debris and growth medium. An isolated antigen-binding protein may further be at least partially free of expression system components such as biological molecules from a host cell or of the growth medium thereof. Generally, the term “isolated” is not intended to refer to a complete absence of such biological molecules (e.g., minor or insignificant amounts of impurity may remain) or to an absence of water, buffers, or salts or to components of a pharmaceutical formulation that includes the antigen-binding proteins (e.g., antibodies or antigen-binding fragments).

An anti-TfR antigen-binding protein of the present invention may be a monoclonal antibody or an antigen-binding fragment of a monoclonal antibody which may be tethered to GAA. The present invention includes monoclonal anti-TfR antigen-binding proteins, e.g., antibodies and antigen-binding fragments thereof, as well as monoclonal compositions comprising a plurality of isolated monoclonal antigen-binding proteins. The term “monoclonal antibody” or “mAb,” as used herein, refers to a member of a population of substantially homogeneous antibodies, i.e., the antibody molecules comprising the population are identical in amino acid sequence except for possible naturally occurring mutations that may be present in minor amounts. A “plurality” of such monoclonal antibodies and fragments in a composition refers to a concentration of identical (i.e., as discussed above, in amino acid sequence except for possible naturally occurring mutations that may be present in minor amounts) antibodies and fragments which is above that which would normally occur in nature, e.g., in the blood of a host organism such as a mouse or a human.

In an embodiment, an anti-TfR antigen-binding protein, e.g., antibody or antigen-binding fragment (which may be tethered to a Payload) comprises a heavy chain constant domain, e.g., of the type IgA (e.g., IgA1 or IgA2), IgD, IgE, IgG (e.g., IgG1, IgG2, IgG3 and IgG4) or IgM. In an embodiment of the invention, an antigen-binding protein, e.g., antibody or antigen-binding fragment, comprises a light chain constant domain, e.g., of the type kappa or lambda.

Included herein are human anti-TfR antigen-binding proteins which may be tethered to GAA. The term “human” antigen-binding protein, such as an antibody or antigen-binding fragment, as used herein, includes antibodies and fragments having variable and constant regions derived from human germline immunoglobulin sequences whether in a human cell or grafted into a non-human cell, e.g., a mouse cell. See, e.g., US8502018, US6596541 or US5789215. The anti-TfR human mAbs of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include mAbs in which CDR sequences derived from the germline of another mammalian species (e.g., mouse) have been grafted onto human FR sequences. The term includes antibodies recombinantly produced in a non-human mammal or in cells of a non-human mammal. The term is not intended to include natural antibodies directly isolated from a human subject.

Also included herein are anti-TfR chimeric antigen-binding proteins, e.g., antibodies and antigen-binding fragments thereof (which may be tethered to GAA), and methods of use thereof. As used herein, a “chimeric antibody” is an antibody having the variable domain from a first antibody and the constant domain from a second antibody, where the first and second antibodies are from different species. (see, e.g., US4816567; and Morrison et al., (1984) Proc. Natl. Acad. Sci. USA 81: 6851-6855).

The term “recombinant” anti-TfR antigen-binding proteins, such as antibodies or antigen-binding fragments thereof (which may be tethered to GAA), refers to such molecules created, expressed, isolated or obtained by technologies or methods known in the art as recombinant DNA technology which include, e.g., DNA splicing and transgenic expression. The term includes antibodies expressed in a non-human mammal (including transgenic non-human mammals, e.g., transgenic mice), or a cell (e.g., CHO cells) such as a cellular expression system or isolated from a recombinant combinatorial human antibody library.

A “variant” of a polypeptide refers to a polypeptide comprising an amino acid sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical or similar to a referenced amino acid sequence that is set forth herein (e.g., any of SEQ ID NOs: 217-220; 222-225; 227-230; 232-235; 237-240; 242-245; 247-250; 252-255; 257-260; 262-265; 267-270; 272-275; 277-280; 282-285; 287-290; 292-295; 297-300; 302-305; 307-310; 312-315; 317-320; 322-325; 327-330; 332-335; 337-340; 342-345; 347-350; 352-355; 357-360; 362-365; 367-370; 372-375; 377-380; 382-385; 387-390; 392-395; 397-400; 402-405; 407-410; 412-415; 417-420; 422-425; 427-430; 432-435; 437-440; 442-445; 447-450; 452-455; 457-460; 462-465; 467-470; 472-475; 477-480; 482-485; 487-490; 492-495; 497-500; 502-505; 507-510; 512-515; 517-520; 522-525; 527-530; 532-535; 703 (optionally not including the N-terminal MHRPRRRGTRPPPLALLAALLLAARGADA (SEQ ID NO: 673)), 704 (optionally not including the N-terminal MHRPRRRGTRPPPLALLAALLLAARGADA (SEQ ID NO: 673)), 705 (optionally not including the N-terminal MHRPRRRGTRPPPLALLAALLLAARGADA (SEQ ID NO: 673)), 706 (optionally not including the N-terminal MHRPRRRGTRPPPLALLAALLLAARGADA (SEQ ID NO: 673)); 538-573, 603-672, or 675-702); when the comparison is performed by a BLAST algorithm wherein the parameters of the algorithm are selected to give the largest match between the respective sequences over the entire length of the respective reference sequences (e.g., expect threshold: 10; word size: 3; max matches in a query range: 0; BLOSUM 62 matrix; gap costs: existence 11, extension 1; conditional compositional score matrix adjustment) and/or comprising the amino acid sequence but having one or more (e.g., 1, 2, 3, 4, 5, 6, 7, 8, 9 or 10) mutations (e.g., point mutation, insertion, truncation, and/or deletion). Preferably, functional GAA ectodomain.

The following references relate to BLAST algorithms often used for sequence analysis: BLAST ALGORITHMS: Altschul et al. (2005) FEBS J. 272(20): 5101-5109; Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, N.Y.

In an embodiment of the invention, an anti-hTfR:Payload or anti-hTfR:Payload (e.g., in scFv, Fab, antibody or antigen-binding fragment thereof format), e.g., wherein the Payload is human GAA, exhibits one or more of the following characteristics:
Affinity (K_{D}) for binding to human TfR at 25° C. in surface plasmon resonance format of about 41 nM or a higher affinity (e.g., about 1 or 0.1 nM or about 0.18 to about 1.2 nM, or higher);
Affinity (K_{D}) for binding to monkey TfR at 25° C. in surface plasmon resonance format of about 0 nM (no detectable binding) or a higher affinity (e.g., about 20 nM or higher);
Ratio of K_{D} for binding to monkey TfR/human TfR at 25° C. in surface plasmon resonance format of from 0 to 278 (e.g., about 17 or 18);
Blocks about 3, 5, 10 or 13 % hTfR (e.g., Hmm-hTFRC such as REGN2431) binding to Human Holo-Tf when in Fab format (IgG1), e.g., no more than about 45% blocking;
Blocks about 6, 8, 10 or 13 % hTfR (e.g., Hmm-hTFRC such as REGN2431) binding to Human Holo-Tf when in scFv (V_{K}-V_{H}) format, e.g., no more than about 45% blocking;
Blocks about 11, 17, 23 or 26 % hTfR (e.g., Hmm-hTFRC such as REGN2431) binding to Human Holo-Tf when in scFv (V_{H}-V_{L}) format, e.g., no more than about 45% blocking;
Exhibits a ratio of about 1 or greater; 0.67 or greater; 1.08 or greater; 0.91 or greater; 0.65 or greater; 0.55 or greater; 0.50 or greater; 0.27 or greater; 0.72 or greater; 1.05 or greater; 0.49 or greater; 0.29 or greater; 1.29 or greater; 1.72 or greater; 1.79 or greater; 3.08 or greater; 1.24or greater; 0.59 or greater; or 0.47 or greater (or about 1-2 or greater) mature hGAA protein in brain (normalized to that of positive control 8D3:GAA scFv) in mice (e.g., Tfrc^{hum/hum} knock-in mice) administered the molecule via HDD, when in anti-hTfR scFv:hGAA format; or delivers mature human GAA protein to the brain of humans administered said scFv:hGAA molecule;
Exhibits a ratio of about 0.44, 0.05, 1.13 or 0.60 (about 0.1-1.2) mature hGAA protein in brain parenchyma (normalized to that of positive control 8D3:GAA scFv) in mice (e.g., Tfrc^{hum/hum} knock-in mice) administered the molecule via HDD, when in anti-hTfR scFv:hGAA format; or delivers mature human GAA protein to the brain parenchyma of humans administered said scFv:hGAA molecule;
Exhibits a ratio of about 0.67, 1.80, 1.78 or 7.74 (about 1-2) mature hGAA protein in quadriceps (normalized to that of positive control 8D3:GAA scFv) in mice (e.g., Tfrc^{hum/hum} knock-in mice) administered the molecule via HDD, when in anti-hTfR scFv:hGAA format; or delivers mature human GAA protein to the quadricep or other muscle tissue of humans administered said scFv:hGAA molecule;
Exhibits a ratio of about 0.94, 0.49, 0.61 or 1.90 (about 0.1-1.2) mature hGAA protein in brain parenchyma (normalized to that of positive control 8D3:GAA scFv) in mice (e.g., Tfrc^{hum} knock-in mice) administered the molecule via AAV8 liver depot, when in anti-hTfR scFv:hGAA format; or delivers mature human GAA protein to the brain parenchyma of humans administered said scFv:hGAA molecule via viral, e.g., AAV, liver depot or parenterally delivered in protein scFv:hGAA fusion format;
Delivers mature hGAA protein to serum, liver, cerebrum, cerebellum, spinal cord, heart and/or quadricep in mice (e.g., Tfrc^{hum} knock-in mice) administered the molecule via AAV8 liver depot, when in anti-hTfR scFv:hGAA format; or delivers mature human GAA protein to the serum, liver, cerebrum, cerebellum, spinal cord, heart and/or quadricep of humans administered said scFv:hGAA molecule via viral, e.g., AAV, liver depot or parenterally delivered in protein scFv:hGAA fusion format;;
Reduces glycogen stored in cerebrum, cerebellum, spinal cord, heart and/or quadricep in mice (e.g., Tfrc^{hum} knock-in mice) administered the molecule via AAV8 liver depot, when in anti-hTfR scFv:hGAA format; e.g., by at least 75% to greater than 95% or greater than 99%; or reduces glycogen stored in cerebrum, cerebellum, spinal cord, heart and/or quadricep of humans administered said scFv:hGAA molecule via viral, e.g., AAV, liver depot, or parenterally delivered in protein scFv:hGAA fusion format;
Reduces glycogen levels in tissues (e.g., cerebellum) of Gaa^{-/-} / Tfrc^{hum} mice treated with liver-depot AAV8 anti-hTFRC scfv:hGAA (e.g., 4e11vg/kg AAV8) by at least about 90% (e.g., about 95% or more) relative to untreated Gaa^{-/-} / Tfrc^{hum} mice;
Reduces glycogen levels in tissues (e.g., quadricep) of Gaa^{-/-} / Tfrc^{hum} mice treated with liver-depot AAV8 anti-hTFRC scfv:hGAA (e.g., 4e11vg/kg AAV8) by at least about 89% (e.g., about 90% or 91% or more) relative to untreated Gaa^{-/-} / Tfrc^{hum} mice; or of humans treated with the fusion, e.g., by parenteral deliver of the fusion protein;
Does not cause abnormal iron homeostasis when administered (e.g., by HDD or AAV8 episomal liver depot) to Tfrc^{hum} mice; e.g., wherein the mice maintain normal serum, heart, liver and/or spleen iron levels, normal total iron-binding capacity (TIBC), and/or normal hepcidin levels); or when administered to humans, e.g., by parenteral deliver of the fusion protein;
When chromosomally inserted (e.g., into the albumin gene locus) or delivered episomally to a subject (e.g., to a human or Gaa^{-/-}/Tfrc^{hum/hum} mouse), for example, in an AAV8 vector, DNA encoding the fusion causes expression of mature human GAA to serum, liver, cerebrum and/or quadricep; and/or
When chromosomally inserted (e.g., into the albumin gene locus) or delivered episomally (e.g., to a human or Gaa^{-/-}/Tfrc^{hum/hum} mouse), for example, in an AAV8 vector, DNA encoding the fusion reduces glycogen levels in the cerebrum and/or quadricep.
^{∗} Tfrc^{hum} or Tfrc^{hum/hum} are homozygous knock-in mice.

The amino acid sequences of domains in anti-human transferrin receptor antigen-binding proteins of fusions disclosed herein are summarized below in Table 29. For example, anti-human transferrin receptor 1 antibodies and antigen-binding fragments thereof (e.g., scFvs and Fabs) comprising the HCVR and LCVR of the molecules in Table 29; or comprising the CDRs thereof, fused to GAA, are disclosed herein.

As discussed, an anti-hTfR:GAA scFv fusion protein (e.g., 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; or 69263) comprises an optional signal peptide, connected to an scFv (e.g., including a V_{L} and a V_{H} optionally connected by a linker), connected to an option linker, connected to a GAA. For example, the optional signal peptide can be the signal peptide from Mus musculus Ror1 (e.g., consisting of the amino acids

MHRPRRRGTRPPPLALLAALLLAARGADA (SEQ ID NO: 673).

In a particular multidomain therapeutic protein, the TfR-binding delivery domain is an anti-TfR scFv. For example, the scFv can include a V_{L} and a V_{H} optionally connected by a linker.

In one example, the anti-hTfR scFv can comprise: (i) a heavy chain variable region that comprises the HCDR1, HCDR2 and HCDR3 of a HCVR comprising the amino acid sequence set forth in SEQ ID NO: 217, 227, 237, 247, 257, 267, 277, 287, 297, 307, 317, 327, 337, 347, 357, 367, 377, 387, 397, 407, 417, 427, 437, 447, 457, 467, 477, 487, 497, 507, 517, or 527; and/or (ii) a light chain variable region that comprises the LCDR1, LCDR2 and LCDR3 of a LCVR comprising the amino acid sequence set forth in SEQ ID NO: 222, 232, 242, 252, 262, 272, 282, 292, 302, 312, 322, 332, 342, 352, 362, 372, 382, 392, 402, 412, 422, 432, 442, 452, 462, 472, 482, 492, 502, 512, 522, or 532.

In another example, the anti-TfR scFv can comprise: (1) a HCVR comprising the HCDR1, HCDR2 and HCDR3 of a HCVR that comprises the amino acid sequence set forth in SEQ ID NO: 217 (or a variant thereof); and a LCVR comprising the LCDR1, LCDR2 and LCDR3 of a LCVR that comprises the amino acid sequence set forth in SEQ ID NO: 222 (or a variant thereof); (2) a HCVR comprising the HCDR1, HCDR2 and HCDR3 of a HCVR that comprises the amino acid sequence set forth in SEQ ID NO: 227 (or a variant thereof); and a LCVR comprising the LCDR1, LCDR2 and LCDR3 of a LCVR that comprises the amino acid sequence set forth in SEQ ID NO: 232 (or a variant thereof); (3) a HCVR comprising the HCDR1, HCDR2 and HCDR3 of a HCVR that comprises the amino acid sequence set forth in SEQ ID NO: 237 (or a variant thereof); and a LCVR comprising the LCDR1, LCDR2 and LCDR3 of a LCVR that comprises the amino acid sequence set forth in SEQ ID NO: 242 (or a variant thereof); (4) a HCVR comprising the HCDR1, HCDR2 and HCDR3 of a HCVR that comprises the amino acid sequence set forth in SEQ ID NO: 247 (or a variant thereof); and a LCVR comprising the LCDR1, LCDR2 and LCDR3 of a LCVR that comprises the amino acid sequence set forth in SEQ ID NO: 252 (or a variant thereof); (5) a HCVR comprising the HCDR1, HCDR2 and HCDR3 of a HCVR that comprises the amino acid sequence set forth in SEQ ID NO: 257 (or a variant thereof); and a LCVR comprising the LCDR1, LCDR2 and LCDR3 of a LCVR that comprises the amino acid sequence set forth in SEQ ID NO: 262 (or a variant thereof); (6) a HCVR comprising the HCDR1, HCDR2 and HCDR3 of a HCVR that comprises the amino acid sequence set forth in SEQ ID NO: 267 (or a variant thereof); and a LCVR comprising the LCDR1, LCDR2 and LCDR3 of a LCVR that comprises the amino acid sequence set forth in SEQ ID NO: 272 (or a variant thereof); (7) a HCVR comprising the HCDR1, HCDR2 and HCDR3 of a HCVR that comprises the amino acid sequence set forth in SEQ ID NO: 277 (or a variant thereof); and a LCVR comprising the LCDR1, LCDR2 and LCDR3 of a LCVR that comprises the amino acid sequence set forth in SEQ ID NO: 282 (or a variant thereof); (8) a HCVR comprising the HCDR1, HCDR2 and HCDR3 of a HCVR that comprises the amino acid sequence set forth in SEQ ID NO: 287 (or a variant thereof); and a LCVR comprising the LCDR1, LCDR2 and LCDR3 of a LCVR that comprises the amino acid sequence set forth in SEQ ID NO: 292 (or a variant thereof); (9) a HCVR comprising the HCDR1, HCDR2 and HCDR3 of a HCVR that comprises the amino acid sequence set forth in SEQ ID NO: 297 (or a variant thereof); and a LCVR comprising the LCDR1, LCDR2 and LCDR3 of a LCVR that comprises the amino acid sequence set forth in SEQ ID NO: 302 (or a variant thereof); (10) a HCVR comprising the HCDR1, HCDR2 and HCDR3 of a HCVR that comprises the amino acid sequence set forth in SEQ ID NO: 307 (or a variant thereof); and a LCVR comprising the LCDR1, LCDR2 and LCDR3 of a LCVR that comprises the amino acid sequence set forth in SEQ ID NO: 312 (or a variant thereof); (11) a HCVR comprising the HCDR1, HCDR2 and HCDR3 of a HCVR that comprises the amino acid sequence set forth in SEQ ID NO: 317 (or a variant thereof); and a LCVR comprising the LCDR1, LCDR2 and LCDR3 of a LCVR that comprises the amino acid sequence set forth in SEQ ID NO: 322 (or a variant thereof); (12) a HCVR comprising the HCDR1, HCDR2 and HCDR3 of a HCVR that comprises the amino acid sequence set forth in SEQ ID NO: 327 (or a variant thereof); and a LCVR comprising the LCDR1, LCDR2 and LCDR3 of a LCVR that comprises the amino acid sequence set forth in SEQ ID NO: 332 (or a variant thereof); (13) a HCVR comprising the HCDR1, HCDR2 and HCDR3 of a HCVR that comprises the amino acid sequence set forth in SEQ ID NO: 337 (or a variant thereof); and a LCVR comprising the LCDR1, LCDR2 and LCDR3 of a LCVR that comprises the amino acid sequence set forth in SEQ ID NO: 342 (or a variant thereof); (14) a HCVR comprising the HCDR1, HCDR2 and HCDR3 of a HCVR that comprises the amino acid sequence set forth in SEQ ID NO: 347 (or a variant thereof); and a LCVR comprising the LCDR1, LCDR2 and LCDR3 of a LCVR that comprises the amino acid sequence set forth in SEQ ID NO: 352 (or a variant thereof); (15) a HCVR comprising the HCDR1, HCDR2 and HCDR3 of a HCVR that comprises the amino acid sequence set forth in SEQ ID NO: 357 (or a variant thereof); and a LCVR comprising the LCDR1, LCDR2 and LCDR3 of a LCVR that comprises the amino acid sequence set forth in SEQ ID NO: 362 (or a variant thereof); (16) a HCVR comprising the HCDR1, HCDR2 and HCDR3 of a HCVR that comprises the amino acid sequence set forth in SEQ ID NO: 367 (or a variant thereof); and a LCVR comprising the LCDR1, LCDR2 and LCDR3 of a LCVR that comprises the amino acid sequence set forth in SEQ ID NO: 372 (or a variant thereof); (17) a HCVR comprising the HCDR1, HCDR2 and HCDR3 of a HCVR that comprises the amino acid sequence set forth in SEQ ID NO: 377 (or a variant thereof); and a LCVR comprising the LCDR1, LCDR2 and LCDR3 of a LCVR that comprises the amino acid sequence set forth in SEQ ID NO: 382 (or a variant thereof); (18) a HCVR comprising the HCDR1, HCDR2 and HCDR3 of a HCVR that comprises the amino acid sequence set forth in SEQ ID NO: 387 (or a variant thereof); and a LCVR comprising the LCDR1, LCDR2 and LCDR3 of a LCVR that comprises the amino acid sequence set forth in SEQ ID NO: 392 (or a variant thereof); (19) a HCVR comprising the HCDR1, HCDR2 and HCDR3 of a HCVR that comprises the amino acid sequence set forth in SEQ ID NO: 397 (or a variant thereof); and a LCVR comprising the LCDR1, LCDR2 and LCDR3 of a LCVR that comprises the amino acid sequence set forth in SEQ ID NO: 402 (or a variant thereof); (20) a HCVR comprising the HCDR1, HCDR2 and HCDR3 of a HCVR that comprises the amino acid sequence set forth in SEQ ID NO: 407 (or a variant thereof); and a LCVR comprising the LCDR1, LCDR2 and LCDR3 of a LCVR that comprises the amino acid sequence set forth in SEQ ID NO: 412 (or a variant thereof); (21) a HCVR comprising the HCDR1, HCDR2 and HCDR3 of a HCVR that comprises the amino acid sequence set forth in SEQ ID NO: 417 (or a variant thereof); and a LCVR comprising the LCDR1, LCDR2 and LCDR3 of a LCVR that comprises the amino acid sequence set forth in SEQ ID NO: 422 (or a variant thereof); (22) a HCVR comprising the HCDR1, HCDR2 and HCDR3 of a HCVR that comprises the amino acid sequence set forth in SEQ ID NO: 427 (or a variant thereof); and a LCVR comprising the LCDR1, LCDR2 and LCDR3 of a LCVR that comprises the amino acid sequence set forth in SEQ ID NO: 432 (or a variant thereof); (23) a HCVR comprising the HCDR1, HCDR2 and HCDR3 of a HCVR that comprises the amino acid sequence set forth in SEQ ID NO: 437 (or a variant thereof); and a LCVR comprising the LCDR1, LCDR2 and LCDR3 of a LCVR that comprises the amino acid sequence set forth in SEQ ID NO: 442 (or a variant thereof); (24) a HCVR comprising the HCDR1, HCDR2 and HCDR3 of a HCVR that comprises the amino acid sequence set forth in SEQ ID NO: 447 (or a variant thereof); and a LCVR comprising the LCDR1, LCDR2 and LCDR3 of a LCVR that comprises the amino acid sequence set forth in SEQ ID NO: 452 (or a variant thereof); (25) a HCVR comprising the HCDR1, HCDR2 and HCDR3 of a HCVR that comprises the amino acid sequence set forth in SEQ ID NO: 457 (or a variant thereof); and a LCVR comprising the LCDR1, LCDR2 and LCDR3 of a LCVR that comprises the amino acid sequence set forth in SEQ ID NO: 462 (or a variant thereof); (26) a HCVR comprising the HCDR1, HCDR2 and HCDR3 of a HCVR that comprises the amino acid sequence set forth in SEQ ID NO: 467 (or a variant thereof); and a LCVR comprising the LCDR1, LCDR2 and LCDR3 of a LCVR that comprises the amino acid sequence set forth in SEQ ID NO: 472 (or a variant thereof); (27) a HCVR comprising the HCDR1, HCDR2 and HCDR3 of a HCVR that comprises the amino acid sequence set forth in SEQ ID NO: 477 (or a variant thereof); and a LCVR comprising the LCDR1, LCDR2 and LCDR3 of a LCVR that comprises the amino acid sequence set forth in SEQ ID NO: 482 (or a variant thereof); (28) a HCVR comprising the HCDR1, HCDR2 and HCDR3 of a HCVR that comprises the amino acid sequence set forth in SEQ ID NO: 487 (or a variant thereof); and a LCVR comprising the LCDR1, LCDR2 and LCDR3 of a LCVR that comprises the amino acid sequence set forth in SEQ ID NO: 492 (or a variant thereof); (29) a HCVR comprising the HCDR1, HCDR2 and HCDR3 of a HCVR that comprises the amino acid sequence set forth in SEQ ID NO: 497 (or a variant thereof); and a LCVR comprising the LCDR1, LCDR2 and LCDR3 of a LCVR that comprises the amino acid sequence set forth in SEQ ID NO: 502 (or a variant thereof); (30) a HCVR comprising the HCDR1, HCDR2 and HCDR3 of a HCVR that comprises the amino acid sequence set forth in SEQ ID NO: 507 (or a variant thereof); and a LCVR comprising the LCDR1, LCDR2 and LCDR3 of a LCVR that comprises the amino acid sequence set forth in SEQ ID NO: 512 (or a variant thereof); (31) a HCVR comprising the HCDR1, HCDR2 and HCDR3 of a HCVR that comprises the amino acid sequence set forth in SEQ ID NO: 517 (or a variant thereof); and a LCVR comprising the LCDR1, LCDR2 and LCDR3 of a LCVR that comprises the amino acid sequence set forth in SEQ ID NO: 522 (or a variant thereof); or (32) a HCVR comprising the HCDR1, HCDR2 and HCDR3 of a HCVR that comprises the amino acid sequence set forth in SEQ ID NO: 527 (or a variant thereof); and a LCVR comprising the LCDR1, LCDR2 and LCDR3 of a LCVR that comprises the amino acid sequence set forth in SEQ ID NO: 532 (or a variant thereof). A variant refers to a polypeptide comprising an amino acid sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical or similar to a referenced amino acid sequence that is set forth herein.

In another example, the anti-TfR scFv can comprise: (a) a HCVR that comprises: an HCDR1 comprising the amino acid sequence set forth in SEQ ID NO: 218 (or a variant thereof), an HCDR2 comprising the amino acid sequence set forth in SEQ ID NO: 219 (or a variant thereof), and an HCDR3 comprising the amino acid sequence set forth in SEQ ID NO: 220 (or a variant thereof); and a LCVR that comprises: an LCDR1 comprising the amino acid sequence set forth in SEQ ID NO: 223 (or a variant thereof), an LCDR2 comprising the amino acid sequence set forth in SEQ ID NO: 224 (or a variant thereof), and an LCDR3 comprising the amino acid sequence set forth in SEQ ID NO: 225 (or a variant thereof); (b) a HCVR that comprises: an HCDR1 comprising the amino acid sequence set forth in SEQ ID NO: 228 (or a variant thereof), an HCDR2 comprising the amino acid sequence set forth in SEQ ID NO: 229 (or a variant thereof), and an HCDR3 comprising the amino acid sequence set forth in SEQ ID NO: 230 (or a variant thereof); and a LCVR that comprises: an LCDR1 comprising the amino acid sequence set forth in SEQ ID NO: 233 (or a variant thereof), an LCDR2 comprising the amino acid sequence set forth in SEQ ID NO: 234 (or a variant thereof), and an LCDR3 comprising the amino acid sequence set forth in SEQ ID NO: 235 (or a variant thereof); (c) a HCVR that comprises: an HCDR1 comprising the amino acid sequence set forth in SEQ ID NO: 238 (or a variant thereof), an HCDR2 comprising the amino acid sequence set forth in SEQ ID NO: 239 (or a variant thereof), and an HCDR3 comprising the amino acid sequence set forth in SEQ ID NO: 240 (or a variant thereof); and a LCVR that comprises: an LCDR1 comprising the amino acid sequence set forth in SEQ ID NO: 243 (or a variant thereof), an LCDR2 comprising the amino acid sequence set forth in SEQ ID NO: 244 (or a variant thereof), and an LCDR3 comprising the amino acid sequence set forth in SEQ ID NO: 245 (or a variant thereof); (d) a HCVR that comprises: an HCDR1 comprising the amino acid sequence set forth in SEQ ID NO: 248 (or a variant thereof), an HCDR2 comprising the amino acid sequence set forth in SEQ ID NO: 249 (or a variant thereof), and an HCDR3 comprising the amino acid sequence set forth in SEQ ID NO: 250 (or a variant thereof); and a LCVR that comprises: an LCDR1 comprising the amino acid sequence set forth in SEQ ID NO: 253 (or a variant thereof), an LCDR2 comprising the amino acid sequence set forth in SEQ ID NO: 254 (or a variant thereof), and an LCDR3 comprising the amino acid sequence set forth in SEQ ID NO: 255 (or a variant thereof); (e) a HCVR that comprises: an HCDR1 comprising the amino acid sequence set forth in SEQ ID NO: 258 (or a variant thereof), an HCDR2 comprising the amino acid sequence set forth in SEQ ID NO: 259 (or a variant thereof), and an HCDR3 comprising the amino acid sequence set forth in SEQ ID NO: 260 (or a variant thereof); and a LCVR that comprises: an LCDR1 comprising the amino acid sequence set forth in SEQ ID NO: 263 (or a variant thereof), an LCDR2 comprising the amino acid sequence set forth in SEQ ID NO: 264 (or a variant thereof), and an LCDR3 comprising the amino acid sequence set forth in SEQ ID NO: 265 (or a variant thereof); (f) a HCVR that comprises: an HCDR1 comprising the amino acid sequence set forth in SEQ ID NO: 268 (or a variant thereof), an HCDR2 comprising the amino acid sequence set forth in SEQ ID NO: 269 (or a variant thereof), and an HCDR3 comprising the amino acid sequence set forth in SEQ ID NO: 270 (or a variant thereof); and a LCVR that comprises: an LCDR1 comprising the amino acid sequence set forth in SEQ ID NO: 273 (or a variant thereof), an LCDR2 comprising the amino acid sequence set forth in SEQ ID NO: 274 (or a variant thereof), and an LCDR3 comprising the amino acid sequence set forth in SEQ ID NO: 275 (or a variant thereof); (g) a HCVR that comprises: an HCDR1 comprising the amino acid sequence set forth in SEQ ID NO: 278 (or a variant thereof), an HCDR2 comprising the amino acid sequence set forth in SEQ ID NO: 279 (or a variant thereof), and an HCDR3 comprising the amino acid sequence set forth in SEQ ID NO: 280 (or a variant thereof); and a LCVR that comprises: an LCDR1 comprising the amino acid sequence set forth in SEQ ID NO: 283 (or a variant thereof), an LCDR2 comprising the amino acid sequence set forth in SEQ ID NO: 284 (or a variant thereof), and an LCDR3 comprising the amino acid sequence set forth in SEQ ID NO: 285 (or a variant thereof); (h) a HCVR that comprises: an HCDR1 comprising the amino acid sequence set forth in SEQ ID NO: 288 (or a variant thereof), an HCDR2 comprising the amino acid sequence set forth in SEQ ID NO: 289 (or a variant thereof), and an HCDR3 comprising the amino acid sequence set forth in SEQ ID NO: 290 (or a variant thereof); and a LCVR that comprises: an LCDR1 comprising the amino acid sequence set forth in SEQ ID NO: 293 (or a variant thereof), an LCDR2 comprising the amino acid sequence set forth in SEQ ID NO: 294 (or a variant thereof), and an LCDR3 comprising the amino acid sequence set forth in SEQ ID NO: 295 (or a variant thereof); (i) a HCVR that comprises: an HCDR1 comprising the amino acid sequence set forth in SEQ ID NO: 298 (or a variant thereof), an HCDR2 comprising the amino acid sequence set forth in SEQ ID NO: 299 (or a variant thereof), and an HCDR3 comprising the amino acid sequence set forth in SEQ ID NO: 300 (or a variant thereof); and a LCVR that comprises: an LCDR1 comprising the amino acid sequence set forth in SEQ ID NO: 303 (or a variant thereof), an LCDR2 comprising the amino acid sequence set forth in SEQ ID NO: 304 (or a variant thereof), and an LCDR3 comprising the amino acid sequence set forth in SEQ ID NO: 305 (or a variant thereof); (j) a HCVR that comprises: an HCDR1 comprising the amino acid sequence set forth in SEQ ID NO: 308 (or a variant thereof), an HCDR2 comprising the amino acid sequence set forth in SEQ ID NO: 309 (or a variant thereof), and an HCDR3 comprising the amino acid sequence set forth in SEQ ID NO: 310 (or a variant thereof); and a LCVR that comprises: an LCDR1 comprising the amino acid sequence set forth in SEQ ID NO: 313 (or a variant thereof), an LCDR2 comprising the amino acid sequence set forth in SEQ ID NO: 314 (or a variant thereof), and an LCDR3 comprising the amino acid sequence set forth in SEQ ID NO: 315 (or a variant thereof); (k) a HCVR that comprises: an HCDR1 comprising the amino acid sequence set forth in SEQ ID NO: 318 (or a variant thereof), an HCDR2 comprising the amino acid sequence set forth in SEQ ID NO: 319 (or a variant thereof), and an HCDR3 comprising the amino acid sequence set forth in SEQ ID NO: 320 (or a variant thereof); and a LCVR that comprises: an LCDR1 comprising the amino acid sequence set forth in SEQ ID NO: 323 (or a variant thereof), an LCDR2 comprising the amino acid sequence set forth in SEQ ID NO: 324 (or a variant thereof), and an LCDR3 comprising the amino acid sequence set forth in SEQ ID NO: 325 (or a variant thereof); (1) a HCVR that comprises: an HCDR1 comprising the amino acid sequence set forth in SEQ ID NO: 328 (or a variant thereof), an HCDR2 comprising the amino acid sequence set forth in SEQ ID NO: 329 (or a variant thereof), and an HCDR3 comprising the amino acid sequence set forth in SEQ ID NO: 330 (or a variant thereof); and a LCVR that comprises: an LCDR1 comprising the amino acid sequence set forth in SEQ ID NO: 333 (or a variant thereof), an LCDR2 comprising the amino acid sequence set forth in SEQ ID NO: 334 (or a variant thereof), and an LCDR3 comprising the amino acid sequence set forth in SEQ ID NO: 335 (or a variant thereof); (m) a HCVR that comprises: an HCDR1 comprising the amino acid sequence set forth in SEQ ID NO: 338 (or a variant thereof), an HCDR2 comprising the amino acid sequence set forth in SEQ ID NO: 339 (or a variant thereof), and an HCDR3 comprising the amino acid sequence set forth in SEQ ID NO: 340 (or a variant thereof); and a LCVR that comprises: an LCDR1 comprising the amino acid sequence set forth in SEQ ID NO: 343 (or a variant thereof), an LCDR2 comprising the amino acid sequence set forth in SEQ ID NO: 344 (or a variant thereof), and an LCDR3 comprising the amino acid sequence set forth in SEQ ID NO: 345 (or a variant thereof); (n) a HCVR that comprises: an HCDR1 comprising the amino acid sequence set forth in SEQ ID NO: 348 (or a variant thereof), an HCDR2 comprising the amino acid sequence set forth in SEQ ID NO: 349 (or a variant thereof), and an HCDR3 comprising the amino acid sequence set forth in SEQ ID NO: 350 (or a variant thereof); and a LCVR that comprises: an LCDR1 comprising the amino acid sequence set forth in SEQ ID NO: 353 (or a variant thereof), an LCDR2 comprising the amino acid sequence set forth in SEQ ID NO: 354 (or a variant thereof), and an LCDR3 comprising the amino acid sequence set forth in SEQ ID NO: 355 (or a variant thereof); (o) a HCVR that comprises: an HCDR1 comprising the amino acid sequence set forth in SEQ ID NO: 358 (or a variant thereof), an HCDR2 comprising the amino acid sequence set forth in SEQ ID NO: 359 (or a variant thereof), and an HCDR3 comprising the amino acid sequence set forth in SEQ ID NO: 360 (or a variant thereof); and a LCVR that comprises: an LCDR1 comprising the amino acid sequence set forth in SEQ ID NO: 363 (or a variant thereof), an LCDR2 comprising the amino acid sequence set forth in SEQ ID NO: 364 (or a variant thereof), and an LCDR3 comprising the amino acid sequence set forth in SEQ ID NO: 365 (or a variant thereof); (p) a HCVR that comprises: an HCDR1 comprising the amino acid sequence set forth in SEQ ID NO: 368 (or a variant thereof), an HCDR2 comprising the amino acid sequence set forth in SEQ ID NO: 369 (or a variant thereof), and an HCDR3 comprising the amino acid sequence set forth in SEQ ID NO: 370 (or a variant thereof); and a LCVR that comprises: an LCDR1 comprising the amino acid sequence set forth in SEQ ID NO: 373 (or a variant thereof), an LCDR2 comprising the amino acid sequence set forth in SEQ ID NO: 374 (or a variant thereof), and an LCDR3 comprising the amino acid sequence set forth in SEQ ID NO: 375 (or a variant thereof); (q) a HCVR that comprises: an HCDR1 comprising the amino acid sequence set forth in SEQ ID NO: 378 (or a variant thereof), an HCDR2 comprising the amino acid sequence set forth in SEQ ID NO: 379 (or a variant thereof), and an HCDR3 comprising the amino acid sequence set forth in SEQ ID NO: 380 (or a variant thereof); and a LCVR that comprises: an LCDR1 comprising the amino acid sequence set forth in SEQ ID NO: 383 (or a variant thereof), an LCDR2 comprising the amino acid sequence set forth in SEQ ID NO: 384 (or a variant thereof), and an LCDR3 comprising the amino acid sequence set forth in SEQ ID NO: 385 (or a variant thereof); (r) a HCVR that comprises: an HCDR1 comprising the amino acid sequence set forth in SEQ ID NO: 388 (or a variant thereof), an HCDR2 comprising the amino acid sequence set forth in SEQ ID NO: 389 (or a variant thereof), and an HCDR3 comprising the amino acid sequence set forth in SEQ ID NO: 390 (or a variant thereof); and a LCVR that comprises: an LCDR1 comprising the amino acid sequence set forth in SEQ ID NO: 393 (or a variant thereof), an LCDR2 comprising the amino acid sequence set forth in SEQ ID NO: 394 (or a variant thereof), and an LCDR3 comprising the amino acid sequence set forth in SEQ ID NO: 395 (or a variant thereof); (s) a HCVR that comprises: an HCDR1 comprising the amino acid sequence set forth in SEQ ID NO: 398 (or a variant thereof), an HCDR2 comprising the amino acid sequence set forth in SEQ ID NO: 399 (or a variant thereof), and an HCDR3 comprising the amino acid sequence set forth in SEQ ID NO: 400 (or a variant thereof); and a LCVR that comprises: an LCDR1 comprising the amino acid sequence set forth in SEQ ID NO: 403 (or a variant thereof), an LCDR2 comprising the amino acid sequence set forth in SEQ ID NO: 404 (or a variant thereof), and an LCDR3 comprising the amino acid sequence set forth in SEQ ID NO: 405 (or a variant thereof); (t) a HCVR that comprises: an HCDR1 comprising the amino acid sequence set forth in SEQ ID NO: 408 (or a variant thereof), an HCDR2 comprising the amino acid sequence set forth in SEQ ID NO: 409 (or a variant thereof), and an HCDR3 comprising the amino acid sequence set forth in SEQ ID NO: 410 (or a variant thereof); and a LCVR that comprises: an LCDR1 comprising the amino acid sequence set forth in SEQ ID NO: 413 (or a variant thereof), an LCDR2 comprising the amino acid sequence set forth in SEQ ID NO: 414 (or a variant thereof), and an LCDR3 comprising the amino acid sequence set forth in SEQ ID NO: 415 (or a variant thereof); (u) a HCVR that comprises: an HCDR1 comprising the amino acid sequence set forth in SEQ ID NO: 418 (or a variant thereof), an HCDR2 comprising the amino acid sequence set forth in SEQ ID NO: 419 (or a variant thereof), and an HCDR3 comprising the amino acid sequence set forth in SEQ ID NO: 420 (or a variant thereof); and a LCVR that comprises: an LCDR1 comprising the amino acid sequence set forth in SEQ ID NO: 423 (or a variant thereof), an LCDR2 comprising the amino acid sequence set forth in SEQ ID NO: 424 (or a variant thereof), and an LCDR3 comprising the amino acid sequence set forth in SEQ ID NO: 425 (or a variant thereof); (v) a HCVR that comprises: an HCDR1 comprising the amino acid sequence set forth in SEQ ID NO: 428 (or a variant thereof), an HCDR2 comprising the amino acid sequence set forth in SEQ ID NO: 429 (or a variant thereof), and an HCDR3 comprising the amino acid sequence set forth in SEQ ID NO: 430 (or a variant thereof); and a LCVR that comprises: an LCDR1 comprising the amino acid sequence set forth in SEQ ID NO: 433 (or a variant thereof), an LCDR2 comprising the amino acid sequence set forth in SEQ ID NO: 434 (or a variant thereof), and an LCDR3 comprising the amino acid sequence set forth in SEQ ID NO: 435 (or a variant thereof); (w) a HCVR that comprises: an HCDR1 comprising the amino acid sequence set forth in SEQ ID NO: 438 (or a variant thereof), an HCDR2 comprising the amino acid sequence set forth in SEQ ID NO: 439 (or a variant thereof), and an HCDR3 comprising the amino acid sequence set forth in SEQ ID NO: 440 (or a variant thereof); and a LCVR that comprises: an LCDR1 comprising the amino acid sequence set forth in SEQ ID NO: 443 (or a variant thereof), an LCDR2 comprising the amino acid sequence set forth in SEQ ID NO: 444 (or a variant thereof), and an LCDR3 comprising the amino acid sequence set forth in SEQ ID NO: 445 (or a variant thereof); (x) a HCVR that comprises: an HCDR1 comprising the amino acid sequence set forth in SEQ ID NO: 448 (or a variant thereof), an HCDR2 comprising the amino acid sequence set forth in SEQ ID NO: 449 (or a variant thereof), and an HCDR3 comprising the amino acid sequence set forth in SEQ ID NO: 450 (or a variant thereof); and a LCVR that comprises: an LCDR1 comprising the amino acid sequence set forth in SEQ ID NO: 453 (or a variant thereof), an LCDR2 comprising the amino acid sequence set forth in SEQ ID NO: 454 (or a variant thereof), and an LCDR3 comprising the amino acid sequence set forth in SEQ ID NO: 455 (or a variant thereof); (y) a HCVR that comprises: an HCDR1 comprising the amino acid sequence set forth in SEQ ID NO: 458 (or a variant thereof), an HCDR2 comprising the amino acid sequence set forth in SEQ ID NO: 459 (or a variant thereof), and an HCDR3 comprising the amino acid sequence set forth in SEQ ID NO: 460 (or a variant thereof); and a LCVR that comprises: an LCDR1 comprising the amino acid sequence set forth in SEQ ID NO: 463 (or a variant thereof), an LCDR2 comprising the amino acid sequence set forth in SEQ ID NO: 464 (or a variant thereof), and an LCDR3 comprising the amino acid sequence set forth in SEQ ID NO: 465 (or a variant thereof); (z) a HCVR that comprises: an HCDR1 comprising the amino acid sequence set forth in SEQ ID NO: 468 (or a variant thereof), an HCDR2 comprising the amino acid sequence set forth in SEQ ID NO: 469 (or a variant thereof), and an HCDR3 comprising the amino acid sequence set forth in SEQ ID NO: 470 (or a variant thereof); and a LCVR that comprises: an LCDR1 comprising the amino acid sequence set forth in SEQ ID NO: 473 (or a variant thereof), an LCDR2 comprising the amino acid sequence set forth in SEQ ID NO: 474 (or a variant thereof), and an LCDR3 comprising the amino acid sequence set forth in SEQ ID NO: 475 (or a variant thereof); (aa) a HCVR that comprises: an HCDR1 comprising the amino acid sequence set forth in SEQ ID NO: 478 (or a variant thereof), an HCDR2 comprising the amino acid sequence set forth in SEQ ID NO: 479 (or a variant thereof), and an HCDR3 comprising the amino acid sequence set forth in SEQ ID NO: 480 (or a variant thereof); and a LCVR that comprises: an LCDR1 comprising the amino acid sequence set forth in SEQ ID NO: 483 (or a variant thereof), an LCDR2 comprising the amino acid sequence set forth in SEQ ID NO: 484 (or a variant thereof), and an LCDR3 comprising the amino acid sequence set forth in SEQ ID NO: 485 (or a variant thereof); (ab) a HCVR that comprises: an HCDR1 comprising the amino acid sequence set forth in SEQ ID NO: 488 (or a variant thereof), an HCDR2 comprising the amino acid sequence set forth in SEQ ID NO: 489 (or a variant thereof), and an HCDR3 comprising the amino acid sequence set forth in SEQ ID NO: 490 (or a variant thereof); and a LCVR that comprises: an LCDR1 comprising the amino acid sequence set forth in SEQ ID NO: 493 (or a variant thereof), an LCDR2 comprising the amino acid sequence set forth in SEQ ID NO: 494 (or a variant thereof), and an LCDR3 comprising the amino acid sequence set forth in SEQ ID NO: 495 (or a variant thereof); (ac) a HCVR that comprises: an HCDR1 comprising the amino acid sequence set forth in SEQ ID NO: 498 (or a variant thereof), an HCDR2 comprising the amino acid sequence set forth in SEQ ID NO: 499 (or a variant thereof), and an HCDR3 comprising the amino acid sequence set forth in SEQ ID NO: 500 (or a variant thereof); and a LCVR that comprises: an LCDR1 comprising the amino acid sequence set forth in SEQ ID NO: 503 (or a variant thereof), an LCDR2 comprising the amino acid sequence set forth in SEQ ID NO: 504 (or a variant thereof), and an LCDR3 comprising the amino acid sequence set forth in SEQ ID NO: 505 (or a variant thereof); (ad) a HCVR that comprises: an HCDR1 comprising the amino acid sequence set forth in SEQ ID NO: 508 (or a variant thereof), an HCDR2 comprising the amino acid sequence set forth in SEQ ID NO: 509 (or a variant thereof), and an HCDR3 comprising the amino acid sequence set forth in SEQ ID NO: 510 (or a variant thereof); and a LCVR that comprises: an LCDR1 comprising the amino acid sequence set forth in SEQ ID NO: 513 (or a variant thereof), an LCDR2 comprising the amino acid sequence set forth in SEQ ID NO: 514 (or a variant thereof), and an LCDR3 comprising the amino acid sequence set forth in SEQ ID NO: 515 (or a variant thereof); (ae) a HCVR that comprises: an HCDR1 comprising the amino acid sequence set forth in SEQ ID NO: 518 (or a variant thereof), an HCDR2 comprising the amino acid sequence set forth in SEQ ID NO: 519 (or a variant thereof), and an HCDR3 comprising the amino acid sequence set forth in SEQ ID NO: 520 (or a variant thereof); and a LCVR that comprises: an LCDR1 comprising the amino acid sequence set forth in SEQ ID NO: 523 (or a variant thereof), an LCDR2 comprising the amino acid sequence set forth in SEQ ID NO: 524 (or a variant thereof), and an LCDR3 comprising the amino acid sequence set forth in SEQ ID NO: 525 (or a variant thereof); and/or (af) a HCVR that comprises: an HCDR1 comprising the amino acid sequence set forth in SEQ ID NO: 528 (or a variant thereof), an HCDR2 comprising the amino acid sequence set forth in SEQ ID NO: 529 (or a variant thereof), and an HCDR3 comprising the amino acid sequence set forth in SEQ ID NO: 530 (or a variant thereof); and a LCVR that comprises: an LCDR1 comprising the amino acid sequence set forth in SEQ ID NO: 533 (or a variant thereof), an LCDR2 comprising the amino acid sequence set forth in SEQ ID NO: 534 (or a variant thereof), and an LCDR3 comprising the amino acid sequence set forth in SEQ ID NO: 535 (or a variant thereof). A variant refers to a polypeptide comprising an amino acid sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical or similar to a referenced amino acid sequence that is set forth herein.

In another example, the anti-TfR scFv can comprise: (i) a HCVR that comprises the amino acid sequence set forth in SEQ ID NO: 217 (or a variant thereof); and a LCVR that comprises the amino acid sequence set forth in SEQ ID NO: 222 (or a variant thereof); (ii) a HCVR that comprises the amino acid sequence set forth in SEQ ID NO: 227 (or a variant thereof); and a LCVR that comprises the amino acid sequence set forth in SEQ ID NO: 232 (or a variant thereof); (iii) a HCVR that comprises the amino acid sequence set forth in SEQ ID NO: 237 (or a variant thereof); and a LCVR that comprises the amino acid sequence set forth in SEQ ID NO: 242 (or a variant thereof); (iv) a HCVR that comprises the amino acid sequence set forth in SEQ ID NO: 247 (or a variant thereof); and a LCVR that comprises the amino acid sequence set forth in SEQ ID NO: 252 (or a variant thereof); (v) a HCVR that comprises the amino acid sequence set forth in SEQ ID NO: 257 (or a variant thereof); and a LCVR that comprises the amino acid sequence set forth in SEQ ID NO: 262 (or a variant thereof); (vi) a HCVR that comprises the amino acid sequence set forth in SEQ ID NO: 267 (or a variant thereof); and a LCVR that comprises the amino acid sequence set forth in SEQ ID NO: 272 (or a variant thereof); (vii) a HCVR that comprises the amino acid sequence set forth in SEQ ID NO: 277 (or a variant thereof); and a LCVR that comprises the amino acid sequence set forth in SEQ ID NO: 282 (or a variant thereof); (viii) a HCVR that comprises the amino acid sequence set forth in SEQ ID NO: 287 (or a variant thereof); and a LCVR that comprises the amino acid sequence set forth in SEQ ID NO: 292 (or a variant thereof); (ix) a HCVR that comprises the amino acid sequence set forth in SEQ ID NO: 297 (or a variant thereof); and a LCVR that comprises the amino acid sequence set forth in SEQ ID NO: 302 (or a variant thereof); (x) a HCVR that comprises the amino acid sequence set forth in SEQ ID NO: 307 (or a variant thereof); and a LCVR that comprises the amino acid sequence set forth in SEQ ID NO: 312 (or a variant thereof); (xi) a HCVR that comprises the amino acid sequence set forth in SEQ ID NO: 317 (or a variant thereof); and a LCVR that comprises the amino acid sequence set forth in SEQ ID NO: 322 (or a variant thereof); (xii) a HCVR that comprises the amino acid sequence set forth in SEQ ID NO: 327 (or a variant thereof); and a LCVR that comprises the amino acid sequence set forth in SEQ ID NO: 332 (or a variant thereof); (xiii) a HCVR that comprises the amino acid sequence set forth in SEQ ID NO: 337 (or a variant thereof); and a LCVR that comprises the amino acid sequence set forth in SEQ ID NO: 342 (or a variant thereof); (xiv) a HCVR that comprises the amino acid sequence set forth in SEQ ID NO: 347 (or a variant thereof); and a LCVR that comprises the amino acid sequence set forth in SEQ ID NO: 352 (or a variant thereof); (xv) a HCVR that comprises the amino acid sequence set forth in SEQ ID NO: 357 (or a variant thereof); and a LCVR that comprises the amino acid sequence set forth in SEQ ID NO: 362 (or a variant thereof); (xvi) a HCVR that comprises the amino acid sequence set forth in SEQ ID NO: 367 (or a variant thereof); and a LCVR that comprises the amino acid sequence set forth in SEQ ID NO: 372 (or a variant thereof); (xvii) a HCVR that comprises the amino acid sequence set forth in SEQ ID NO: 377 (or a variant thereof); and a LCVR that comprises the amino acid sequence set forth in SEQ ID NO: 382 (or a variant thereof); (xviii) a HCVR that comprises the amino acid sequence set forth in SEQ ID NO: 387 (or a variant thereof); and a LCVR that comprises the amino acid sequence set forth in SEQ ID NO: 392 (or a variant thereof); (xix) a HCVR that comprises the amino acid sequence set forth in SEQ ID NO: 397 (or a variant thereof); and a LCVR that comprises the amino acid sequence set forth in SEQ ID NO: 402 (or a variant thereof); (xx) a HCVR that comprises the amino acid sequence set forth in SEQ ID NO: 407 (or a variant thereof); and a LCVR that comprises the amino acid sequence set forth in SEQ ID NO: 412 (or a variant thereof); (xxi) a HCVR that comprises the amino acid sequence set forth in SEQ ID NO: 417 (or a variant thereof); and a LCVR that comprises the amino acid sequence set forth in SEQ ID NO: 422 (or a variant thereof); (xxii) a HCVR that comprises the amino acid sequence set forth in SEQ ID NO: 427 (or a variant thereof); and a LCVR that comprises the amino acid sequence set forth in SEQ ID NO: 432 (or a variant thereof); (xxiii) a HCVR that comprises the amino acid sequence set forth in SEQ ID NO: 437 (or a variant thereof); and a LCVR that comprises the amino acid sequence set forth in SEQ ID NO: 442 (or a variant thereof); (xxiv) a HCVR that comprises the amino acid sequence set forth in SEQ ID NO: 447 (or a variant thereof); and a LCVR that comprises the amino acid sequence set forth in SEQ ID NO: 452 (or a variant thereof); (xxv) a HCVR that comprises the amino acid sequence set forth in SEQ ID NO: 457 (or a variant thereof); and a LCVR that comprises the amino acid sequence set forth in SEQ ID NO: 462 (or a variant thereof); (xxvi) a HCVR that comprises the amino acid sequence set forth in SEQ ID NO: 467 (or a variant thereof); and a LCVR that comprises the amino acid sequence set forth in SEQ ID NO: 472 (or a variant thereof); (xxvii) a HCVR that comprises the amino acid sequence set forth in SEQ ID NO: 477 (or a variant thereof); and a LCVR that comprises the amino acid sequence set forth in SEQ ID NO: 482 (or a variant thereof); (xxviii) a HCVR that comprises the amino acid sequence set forth in SEQ ID NO: 487 (or a variant thereof); and a LCVR that comprises the amino acid sequence set forth in SEQ ID NO: 492 (or a variant thereof); (xxix) a HCVR that comprises the amino acid sequence set forth in SEQ ID NO: 497 (or a variant thereof); and a LCVR that comprises the amino acid sequence set forth in SEQ ID NO: 502 (or a variant thereof); (xxx) a HCVR that comprises the amino acid sequence set forth in SEQ ID NO: 507 (or a variant thereof); and a LCVR that comprises the amino acid sequence set forth in SEQ ID NO: 512 (or a variant thereof); (xxxi) a HCVR that comprises the amino acid sequence set forth in SEQ ID NO: 517 (or a variant thereof); and a LCVR that comprises the amino acid sequence set forth in SEQ ID NO: 522 (or a variant thereof); and/or (xxxii) a HCVR that comprises the amino acid sequence set forth in SEQ ID NO: 527 (or a variant thereof); and a LCVR that comprises the amino acid sequence set forth in SEQ ID NO: 532 (or a variant thereof), optionally wherein the HCVR and LCVR are linked by a linker (e.g., that comprises an amino acid sequence, e.g., about 10 amino acids in length, for example, 1, 2, 3, 4, 5, 6, 7, 8, 8, or 10 repeats of Gly₄Ser (SEQ ID NO: 600). A variant refers to a polypeptide comprising an amino acid sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical or similar to a referenced amino acid sequence that is set forth herein.

Examples of polynucleotides encoding anti-TfR scFvs are provided in Table 29 and include: (1) a polynucleotide encoding a HCVR that comprises the nucleotide sequence set forth in SEQ ID NO: 216, and a LCVR that comprises the nucleotide sequence set forth in SEQ ID NO: 221; (2) a polynucleotide encoding a HCVR that comprises the nucleotide sequence set forth in SEQ ID NO: 226, and a LCVR that comprises the nucleotide sequence set forth in SEQ ID NO: 231; (3) a polynucleotide encoding a HCVR that comprises the nucleotide sequence set forth in SEQ ID NO: 236, and a LCVR that comprises the nucleotide sequence set forth in SEQ ID NO: 241; (4) a polynucleotide encoding a HCVR that comprises the nucleotide sequence set forth in SEQ ID NO: 246, and a LCVR that comprises the nucleotide sequence set forth in SEQ ID NO: 251; (5) a polynucleotide encoding a HCVR that comprises the nucleotide sequence set forth in SEQ ID NO: 256, and a LCVR that comprises the nucleotide sequence set forth in SEQ ID NO: 261; (6) a polynucleotide encoding a HCVR that comprises the nucleotide sequence set forth in SEQ ID NO: 266, and a LCVR that comprises the nucleotide sequence set forth in SEQ ID NO: 271; (7) a polynucleotide encoding a HCVR that comprises the nucleotide sequence set forth in SEQ ID NO: 276, and a LCVR that comprises the nucleotide sequence set forth in SEQ ID NO: 281; (8) a polynucleotide encoding a HCVR that comprises the nucleotide sequence set forth in SEQ ID NO: 286, and a LCVR that comprises the nucleotide sequence set forth in SEQ ID NO: 291; (9) a polynucleotide encoding a HCVR that comprises the nucleotide sequence set forth in SEQ ID NO: 296, and a LCVR that comprises the nucleotide sequence set forth in SEQ ID NO: 301; (10) a polynucleotide encoding a HCVR that comprises the nucleotide sequence set forth in SEQ ID NO: 306, and a LCVR that comprises the nucleotide sequence set forth in SEQ ID NO: 311; (11) a polynucleotide encoding a HCVR that comprises the nucleotide sequence set forth in SEQ ID NO: 316, and a LCVR that comprises the nucleotide sequence set forth in SEQ ID NO: 321; (12) a polynucleotide encoding a HCVR that comprises the nucleotide sequence set forth in SEQ ID NO: 326, and a LCVR that comprises the nucleotide sequence set forth in SEQ ID NO: 331; (13) a polynucleotide encoding a HCVR that comprises the nucleotide sequence set forth in SEQ ID NO: 336, and a LCVR that comprises the nucleotide sequence set forth in SEQ ID NO: 341; (14) a polynucleotide encoding a HCVR that comprises the nucleotide sequence set forth in SEQ ID NO: 346, and a LCVR that comprises the nucleotide sequence set forth in SEQ ID NO: 351; (15) a polynucleotide encoding a HCVR that comprises the nucleotide sequence set forth in SEQ ID NO: 356, and a LCVR that comprises the nucleotide sequence set forth in SEQ ID NO: 361; (16) a polynucleotide encoding a HCVR that comprises the nucleotide sequence set forth in SEQ ID NO: 366, and a LCVR that comprises the nucleotide sequence set forth in SEQ ID NO: 371; (17) a polynucleotide encoding a HCVR that comprises the nucleotide sequence set forth in SEQ ID NO: 376, and a LCVR that comprises the nucleotide sequence set forth in SEQ ID NO: 381; (18) a polynucleotide encoding a HCVR that comprises the nucleotide sequence set forth in SEQ ID NO: 386, and a LCVR that comprises the nucleotide sequence set forth in SEQ ID NO: 391; (19) a polynucleotide encoding a HCVR that comprises the nucleotide sequence set forth in SEQ ID NO: 396, and a LCVR that comprises the nucleotide sequence set forth in SEQ ID NO: 401; (20) a polynucleotide encoding a HCVR that comprises the nucleotide sequence set forth in SEQ ID NO: 406, and a LCVR that comprises the nucleotide sequence set forth in SEQ ID NO: 411; (21) a polynucleotide encoding a HCVR that comprises the nucleotide sequence set forth in SEQ ID NO: 416, and a LCVR that comprises the nucleotide sequence set forth in SEQ ID NO: 421; (22) a polynucleotide encoding a HCVR that comprises the nucleotide sequence set forth in SEQ ID NO: 426, and a LCVR that comprises the nucleotide sequence set forth in SEQ ID NO: 431; (23) a polynucleotide encoding a HCVR that comprises the nucleotide sequence set forth in SEQ ID NO: 436, and a LCVR that comprises the nucleotide sequence set forth in SEQ ID NO: 441; (24) a polynucleotide encoding a HCVR that comprises the nucleotide sequence set forth in SEQ ID NO: 446, and a LCVR that comprises the nucleotide sequence set forth in SEQ ID NO: 451; (25) a polynucleotide encoding a HCVR that comprises the nucleotide sequence set forth in SEQ ID NO: 456, and a LCVR that comprises the nucleotide sequence set forth in SEQ ID NO: 461; (26) a polynucleotide encoding a HCVR that comprises the nucleotide sequence set forth in SEQ ID NO: 466, and a LCVR that comprises the nucleotide sequence set forth in SEQ ID NO: 471; (27) a polynucleotide encoding a HCVR that comprises the nucleotide sequence set forth in SEQ ID NO: 476, and a LCVR that comprises the nucleotide sequence set forth in SEQ ID NO: 481; (28) a polynucleotide encoding a HCVR that comprises the nucleotide sequence set forth in SEQ ID NO: 486, and a LCVR that comprises the nucleotide sequence set forth in SEQ ID NO: 491; (29) a polynucleotide encoding a HCVR that comprises the nucleotide sequence set forth in SEQ ID NO: 496, and a LCVR that comprises the nucleotide sequence set forth in SEQ ID NO: 501; (30) a polynucleotide encoding a HCVR that comprises the nucleotide sequence set forth in SEQ ID NO: 506, and a LCVR that comprises the nucleotide sequence set forth in SEQ ID NO: 511; (31) a polynucleotide encoding a HCVR that comprises the nucleotide sequence set forth in SEQ ID NO: 516, and a LCVR that comprises the nucleotide sequence set forth in SEQ ID NO: 521; or (32) a polynucleotide encoding a HCVR that comprises the nucleotide sequence set forth in SEQ ID NO: 526, and a LCVR that comprises the nucleotide sequence set forth in SEQ ID NO: 531, wherein the HCVR and LCVR are in either order.

In an embodiment, an anti-hTfR scFv, in V_{L}-(Gly₄Ser)₃-V_{H} format (Gly₄Ser = SEQ ID NO: 600), comprises the amino acid sequence set forth in any one of SEQ ID NOS: 538-569. Also contemplated are such fusions that are in the format V_{H}-(Gly₄Ser)₃-V_{L} (Gly₄Ser = SEQ ID NO: 600).

In another example, the TfR-binding delivery domain can be a Fab fragments (e.g., that binds specifically to human transferrin receptor). Fab fragments typically contain one complete light chain, VL and constant light domain, e.g., kappa (e.g.,

RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC (SEQ ID NO:601)

) and the V_{H} and IgG1 CH1 portion (e.g.,

ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTH (SEQ ID NO:602)

) or IgG4 CH1 (e.g.,

ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPLLQGSG (SEQ IDNO: 674)

) of one heavy chain. Fab fragment antibodies can be generated by papain digestion of whole IgG antibodies to remove the entire Fc fragment, including the hinge region. In one example, the Fab protein can comprise: (1) a heavy chain variable region (HCVR) that comprisesthe amino acid sequence set forth in SEQ ID NO: 217, or a heavy chain variable region that includes HCDR1, HCDR2 and HCDR3 of such a HCVR- linked to the CH1 domain-and a light chain variable region (LCVR) that comprises the amino acid sequence set forth in SEQ ID NO: 222, or LCDR1, LCDR2 and LCDR3 of such a LCVR-linked to the CL domain; (2) a heavy chain variable region (HCVR) that comprises the amino acid sequence set forth in SEQ ID NO: 227, or a heavy chain variable region that includes HCDR1, HCDR2 and HCDR3 of such a HCVR- linked to the CH1 domain-and a light chain variable region (LCVR) that comprises the amino acid sequence set forth in SEQ ID NO: 232, or LCDR1, LCDR2 and LCDR3 of such a LCVR-linked to the CL domain; (3) a heavy chain variable region (HCVR) that comprises the amino acid sequence set forth in SEQ ID NO: 237, or a heavy chain variable region that includes HCDR1, HCDR2 and HCDR3 of such a HCVR- linked to the CH1 domain-and a light chain variable region (LCVR) that comprises the amino acid sequence set forth in SEQ ID NO: 242, or LCDR1, LCDR2 and LCDR3 of such a LCVR-linked to the CL domain; (4) a heavy chain variable region (HCVR) that comprises the amino acid sequence set forth in SEQ ID NO: 247, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 252, or LCDR1, LCDR2 and LCDR3 of such a LCVR-linked to the CL domain; (5) a heavy chain variable region (HCVR) that comprises the amino acid sequence set forth in SEQ ID NO: 257, or a heavy chain variable region that includes HCDR1, HCDR2 and HCDR3 of such a HCVR- linked to the CH1 domain-and a light chain variable region (LCVR) that comprises the amino acid sequence set forth in SEQ ID NO: 262, or LCDR1, LCDR2 and LCDR3 of such a LCVR-linked to the CL domain; (6) a heavy chain variable region (HCVR) that comprises the amino acid sequence set forth in SEQ ID NO: 267, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 272, or LCDR1, LCDR2 and LCDR3 of such a LCVR-linked to the CL domain; (7) a heavy chain variable region (HCVR) that comprises the amino acid sequence set forth in SEQ ID NO: 277, or a heavy chain variable region that includes HCDR1, HCDR2 and HCDR3 of such a HCVR- linked to the CH1 domain-and a light chain variable region (LCVR) that comprises the amino acid sequence set forth in SEQ ID NO: 282, or LCDR1, LCDR2 and LCDR3 of such a LCVR-linked to the CL domain; (8) a heavy chainvariable region (HCVR) that comprises the amino acid sequence set forth in SEQ ID NO: 287, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 292, or LCDR1, LCDR2 and LCDR3 of such a LCVR-linked to the CL domain; (9) a heavy chain variable region (HCVR) that comprises the amino acid sequence set forth in SEQ ID NO: 297, or a heavy chain variable region that includes HCDR1, HCDR2 and HCDR3 of such a HCVR- linked to the CH1 domain-and a light chain variable region (LCVR) that comprises the amino acid sequence set forth in SEQ ID NO: 302, or LCDR1, LCDR2 and LCDR3 of such a LCVR-linked to the CL domain; (10) a heavy chain variable region (HCVR) that comprises the amino acid sequence set forth in SEQ ID NO: 307, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 312, or LCDR1, LCDR2 and LCDR3 of such a LCVR-linked to the CL domain; (11) a heavy chain variable region (HCVR) that comprises the amino acid sequence set forth in SEQ ID NO: 317, or a heavy chain variable region that includes HCDR1, HCDR2 and HCDR3 of such a HCVR- linked to the CH1 domain-and a light chain variable region (LCVR) that comprises the amino acid sequence set forth in SEQ ID NO: 322, or LCDR1, LCDR2 and LCDR3 of such a LCVR-linked to the CL domain; (12) a heavy chain variable region (HCVR) that comprises the amino acid sequence set forth in SEQ ID NO: 327, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 332, or LCDR1, LCDR2 and LCDR3 of such a LCVR-linked to the CL domain; (13) a heavy chain variable region (HCVR) that comprises the amino acid sequence set forth in SEQ ID NO: 337, or a heavy chain variable region that includes HCDR1, HCDR2 and HCDR3 of such a HCVR- linked to the CH1 domain-and a light chain variable region (LCVR) that comprises the amino acid sequence set forth in SEQ ID NO: 342, or LCDR1, LCDR2 and LCDR3 of such a LCVR-linked to the CL domain; (14) a heavy chain variable region (HCVR) that comprises the amino acid sequence set forth in SEQ ID NO: 347, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 352, or LCDR1, LCDR2 and LCDR3 of such a LCVR-linked to the CL domain; (15) a heavy chain variable region (HCVR) that comprises the amino acid sequence set forth in SEQ ID NO: 357, or a heavy chain variable region that includes HCDR1, HCDR2 and HCDR3 of such a HCVR- linked to the CH1 domain-and a light chain variable region (LCVR) that comprises the amino acid sequence set forth in SEQ ID NO: 362, or LCDR1, LCDR2 and LCDR3 of such a LCVR-linked to the CL domain; (16) a heavy chain variable region (HCVR) that comprises the amino acid sequence set forth in SEQ ID NO: 367, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 372, or LCDR1, LCDR2 and LCDR3 of such a LCVR-linked to the CL domain; (17) a heavy chain variable region (HCVR) that comprises the amino acid sequence set forth in SEQ ID NO: 377, or a heavy chain variable region that includes HCDR1, HCDR2 and HCDR3 of such a HCVR- linked to the CH1 domain-and a light chain variable region (LCVR) that comprises the amino acid sequence set forth in SEQ ID NO: 382, or LCDR1, LCDR2 and LCDR3 of such a LCVR-linked to the CL domain; (18) a heavy chain variable region (HCVR) that comprises the amino acid sequence set forth in SEQ ID NO: 387, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 392, or LCDR1, LCDR2 and LCDR3 of such a LCVR-linked to the CL domain; (19) a heavy chain variable region (HCVR) that comprises the amino acid sequence set forth in SEQ ID NO: 397, or a heavy chain variable region that includes HCDR1, HCDR2 and HCDR3 of such a HCVR- linked to the CH1 domain-and a light chain variable region (LCVR) that comprises the amino acid sequence set forth in SEQ ID NO: 402, or LCDR1, LCDR2 and LCDR3 of such a LCVR-linked to the CL domain; (20) a heavy chain variable region (HCVR) that comprises the amino acid sequence set forth in SEQ ID NO: 407, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 412, or LCDR1, LCDR2 and LCDR3 of such a LCVR-linked to the CL domain; (21) a heavy chain variable region (HCVR) that comprises the amino acid sequence set forth in SEQ ID NO: 417, or a heavy chain variable region that includes HCDR1, HCDR2 and HCDR3 of such a HCVR- linked to the CH1 domain-and a light chain variable region (LCVR) that comprises the amino acid sequence set forth in SEQ ID NO: 422, orLCDR1, LCDR2 and LCDR3 of such a LCVR-linked to the CL domain; (22) a heavy chain variable region (HCVR) that comprises the amino acid sequence set forth in SEQ ID NO: 427, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 432, or LCDR1, LCDR2 and LCDR3 of such a LCVR-linked to the CL domain; (23) a heavy chain variable region (HCVR) that comprises the amino acid sequence set forth in SEQ ID NO: 437, or a heavy chain variable region that includes HCDR1, HCDR2 and HCDR3 of such a HCVR- linked to the CH1 domain-and a light chain variable region (LCVR) that comprises the amino acid sequence set forth in SEQ ID NO: 442, or LCDR1, LCDR2 and LCDR3 of such a LCVR-linked to the CL domain; (24) a heavy chain variable region (HCVR) that comprises the amino acid sequence set forth in SEQ ID NO: 447, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 452, or LCDR1, LCDR2 and LCDR3 of such a LCVR-linked to the CL domain; (25) a heavy chain variable region (HCVR) that comprises the amino acid sequence set forth in SEQ ID NO: 457, or a heavy chain variable region that includes HCDR1, HCDR2 and HCDR3 of such a HCVR- linked to the CH1 domain-and a light chain variable region (LCVR) that comprises the amino acid sequence set forth in SEQ ID NO: 462, or LCDR1, LCDR2 and LCDR3 of such a LCVR-linked to the CL domain; (26) a heavy chain variable region (HCVR) that comprises the amino acid sequence set forth in SEQ ID NO: 467, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 472, or LCDR1, LCDR2 and LCDR3 of such a LCVR-linked to the CL domain; (27) a heavy chain variable region (HCVR) that comprises the amino acid sequence set forth in SEQ ID NO: 477, or a heavy chain variable region that includes HCDR1, HCDR2 and HCDR3 of such a HCVR- linked to the CH1 domain-and a light chain variable region (LCVR) that comprises the amino acid sequence set forth in SEQ ID NO: 482, or LCDR1, LCDR2 and LCDR3 of such a LCVR-linked to the CL domain; (28) a heavy chain variable region (HCVR) that comprises the amino acid sequence set forth in SEQ ID NO: 487, or a heavy chain variable region that includes HCDR1, HCDR2 and HCDR3 of such a HCVR-linked to the CH1 domain-and a light chain variable region (LCVR) that comprises the aminoacid sequence set forth in SEQ ID NO: 492, or LCDR1, LCDR2 and LCDR3 of such a LCVR-linked to the CL domain; (29) a heavy chain variable region (HCVR) that comprises the amino acid sequence set forth in SEQ ID NO: 497, or a heavy chain variable region that includes HCDR1, HCDR2 and HCDR3 of such a HCVR- linked to the CH1 domain-and a light chain variable region (LCVR) that comprises the amino acid sequence set forth in SEQ ID NO: 502, or LCDR1, LCDR2 and LCDR3 of such a LCVR-linked to the CL domain; (30) a heavy chain variable region (HCVR) that comprises the amino acid sequence set forth in SEQ ID NO: 507, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 512, or LCDR1, LCDR2 and LCDR3 of such a LCVR-linked to the CL domain; (31) a heavy chain variable region (HCVR) that comprises the amino acid sequence set forth in SEQ ID NO: 517, or a heavy chain variable region that includes HCDR1, HCDR2 and HCDR3 of such a HCVR- linked to the CH1 domain-and a light chain variable region (LCVR) that comprises the amino acid sequence set forth in SEQ ID NO: 522, or LCDR1, LCDR2 and LCDR3 of such a LCVR-linked to the CL domain; and/or (32) a heavy chain variable region (HCVR) that comprises the amino acid sequence set forth in SEQ ID NO: 527, or a heavy chain variable region that includes HCDR1, HCDR2 and HCDR3 of such a HCVR- linked to the CH1 domain-and a light chain variable region (LCVR) that comprises the amino acid sequence set forth in SEQ ID NO: 532, or LCDR1, LCDR2 and LCDR3 of such a LCVR-linked to the CL domain. For example, the CH1 can be SEQ ID NO: 602 or 674.

In one example, the Fab protein can comprise: (1) a light chain that comprises the amino acid sequence set forth in SEQ ID NO: 603 and a heavy chain that comprises the amino acid sequence set forth in SEQ ID NO: 604 (31874B); (2) a light chain that comprises the amino acid sequence set forth in SEQ ID NO: 605 and a heavy chain that comprises the amino acid sequence set forth in SEQ ID NO: 606 (31863B); (3) a light chain that comprises the amino acid sequence set forth in SEQ ID NO: 607 and a heavy chain that comprises the amino acid sequence set forth in SEQ ID NO: 608 (69348); (4) a light chain that comprises the amino acid sequence set forth in SEQ ID NO: 609 and a heavy chain that comprises the amino acid sequence set forth in SEQ ID NO: 610 (69340); (5) a light chain that comprises the amino acid sequence set forth in SEQ ID NO: 611 and a heavy chain that comprises the amino acid sequence set forth in SEQ ID NO: 612 (69331); (6) a light chain that comprises the amino acid sequence set forth in SEQ ID NO: 613 and a heavy chain that comprises the amino acid sequence set forth in SEQ ID NO: 614 (69332); (7) a light chain that comprises the amino acid sequence set forth in SEQ ID NO: 615 and a heavy chain that comprises the amino acid sequence set forth in SEQ ID NO: 616 (69326); (8) a light chain that comprises the amino acid sequence set forth in SEQ ID NO: 617 and a heavy chain that comprises the amino acid sequence set forth in SEQ ID NO: 618 (69329); (9) a light chain that comprises the amino acid sequence set forth in SEQ ID NO: 619 and a heavy chain that comprises the amino acid sequence set forth in SEQ ID NO: 620 (69323); (10) a light chain that comprises the amino acid sequence set forth in SEQ ID NO: 621 and a heavy chain that comprises the amino acid sequence set forth in SEQ ID NO: 622 (69305); (11) a light chain that comprises the amino acid sequence set forth in SEQ ID NO: 623 and a heavy chain that comprises the amino acid sequence set forth in SEQ ID NO: 624 (69307); (12) a light chain that comprises the amino acid sequence set forth in SEQ ID NO: 625 and a heavy chain that comprises the amino acid sequence set forth in SEQ ID NO: 626 (12795B); (13) a light chain that comprises the amino acid sequence set forth in SEQ ID NO: 627 and a heavy chain that comprises the amino acid sequence set forth in SEQ ID NO: 628 or SEQ ID NO: 667 (12798B); (14) a light chain that comprises the amino acid sequence set forth in SEQ ID NO: 629 and a heavy chain that comprises the amino acid sequence set forth in SEQ ID NO: 630 or SEQ ID NO: 668 (12799B); (15) a light chain that comprises the amino acid sequence set forth in SEQ ID NO: 631 and a heavy chain that comprises the amino acid sequence set forth in SEQ ID NO: 632 (12801B); (16) a light chain that comprises the amino acid sequence set forth in SEQ ID NO: 633 and a heavy chain that comprises the amino acid sequence set forth in SEQ ID NO: 634 (12802B); (17) a light chain that comprises the amino acid sequence set forth in SEQ ID NO: 635 and a heavy chain that comprises the amino acid sequence set forth in SEQ ID NO: 636 (12808B); (18) a light chain that comprises the amino acid sequence set forth in SEQ ID NO: 637 and a heavy chain that comprises the amino acid sequence set forth in SEQ ID NO: 638 (12812B); (19) a light chain that comprises the amino acid sequence set forth in SEQ ID NO: 639 and a heavy chain that comprises the amino acid sequence set forth in SEQ ID NO: 640 (12816B); (20) a light chain that comprises the amino acid sequence set forth in SEQ ID NO: 641 and a heavy chain that comprises the amino acid sequence set forth in SEQ ID NO: 642 (12833B); (21) a light chain that comprises the amino acid sequence set forth in SEQ ID NO: 643 and a heavy chain that comprises the amino acid sequence set forth in SEQ ID NO: 644 (12834B); (22) a light chain that comprises the amino acid sequence set forth in SEQ ID NO: 645 and a heavy chain that comprises the amino acid sequence set forth in SEQ ID NO: 646 (12835B); (23) a light chain that comprises the amino acid sequence set forth in SEQ ID NO: 647 and a heavy chain that comprises the amino acid sequence set forth in SEQ ID NO: 648 or SEQ ID NO: 669 (12847B); (24) a light chain that comprises the amino acid sequence set forth in SEQ ID NO: 649 and a heavy chain that comprises the amino acid sequence set forth in SEQ ID NO: 650 (12848B); (25) a light chain that comprises the amino acid sequence set forth in SEQ ID NO: 651 and a heavy chain that comprises the amino acid sequence set forth in SEQ ID NO: 652 or SEQ ID NO: 670 (12843B); (26) a light chain that comprises the amino acid sequence set forth in SEQ ID NO: 653 and a heavy chain that comprises the amino acid sequence set forth in SEQ ID NO: 654 (12844B); (27) a light chain that comprises the amino acid sequence set forth in SEQ ID NO: 655 and a heavy chain that comprises the amino acid sequence set forth in SEQ ID NO: 656 or SEQ ID NO: 671 (12845B); (28) a light chain that comprises the amino acid sequence set forth in SEQ ID NO: 657 and a heavy chain that comprises the amino acid sequence set forth in SEQ ID NO: 658 or SEQ ID NO: 672 (12839B); (29) a light chain that comprises the amino acid sequence set forth in SEQ ID NO: 659 and a heavy chain that comprises the amino acid sequence set forth in SEQ ID NO: 660 (12841B); (30) a light chain that comprises the amino acid sequence set forth in SEQ ID NO: 661 and a heavy chain that comprises the amino acid sequence set forth in SEQ ID NO: 662 (12850B); (31) a light chain that comprises the amino acid sequence set forth in SEQ ID NO: 663 and a heavy chain that comprises the amino acid sequence set forth in SEQ ID NO: 664 (69261); or (32) a light chain that comprises the amino acid sequence set forth in SEQ ID NO: 665 and a heavy chain that comprises the amino acid sequence set forth in SEQ ID NO: 666 (69263).

“31874B”; “31863B”; “69348”; “69340”; “69331”; “69332”; “69326”; “69329”; “69323”; “69305”; “69307”; “12795B”; “12798B”; “12799B”; “12801B”; “12802B”; “12808B”; “12812B”; “12816B”; “12833B”; “12834B”; “12835B”; “12847B”; “12848B”; “12843B”; “12844B”; “12845B”; “12839B”; “12841B”; “12850B”; “69261”; and “69263” refer to anti-TfR:GAA fusion proteins, e.g., anti-TfR scFv:GAA or anti-TfR Fab:GAA, comprising a light chain variable region comprising the amino acid sequence set forth in SEQ ID NO: 222, 232, 242, 252, 262, 272, 282, 292, 302, 312, 322, 332, 342, 352, 362, 372, 382, 392, 402, 412, 422, 432, 442, 452, 462, 472, 482, 492, 502, 512, 522, or 532 (or a variant thereof), and a heavy chain variable region comprising the amino acid sequence set forth in SEQ ID NO: 217, 227, 237, 247, 257, 267, 277, 287, 297, 307, 317, 327, 337, 347, 357, 367, 377, 387, 397, 407, 417, 427, 437, 447, 457, 467, 477, 487, 497, 507, 517, or 527 (or a variant thereof); which, in the case of an scFv, can be fused together (in either order), e.g., by a peptide linker (e.g., (G₄S)₃) (G₄S = SEQ ID NO: 600), respectively; or that comprise a V_{H} that comprises the CDRs thereof (CDR-H1 (or a variant thereof), CDR-H2 (or a variant thereof) and CDR-H3 (or a variant thereof)) and/or a V_{L} that comprises the CDRs thereof (CDR-L1 (or a variant thereof), CDR-L2 (or a variant thereof) and CDR-L3 (or a variant thereof)), wherein the V_{H} fused to the V_{L} or the V_{L} fused to the V_{H}, in the case of an scFv, can be fused, e.g., by a peptide linker (e.g., (G₄S)₂) (G₄S = SEQ ID NO: 600), to GAA.

The TfR-binding delivery domain coding sequences in the constructs disclosed herein may include one or more modifications such as codon optimization (e.g., to human codons), depletion of CpG dinucleotides, mutation of cryptic splice sites, addition of one or more glycosylation sites, or any combination thereof. CpG dinucleotides in a construct can limit the therapeutic utility of the construct. First, unmethylated CpG dinucleotides can interact with host toll-like receptor-9 (TLR-9) to stimulate innate, proinflammatory immune responses. Second, once the CpG dinucleotides become methylated, they can result in the suppression of transgene expression coordinated by methyl-CpG binding proteins. Cryptic splice sites are sequences in a pre-messenger RNA that are not normally used as splice sites, but that can be activated, for example, by mutations that either inactivate canonical splice sites or create splice sites where one did not exist before. Accurate splice site selection is critical for successful gene expression, and removal of cryptic splice sites can favor use of the normal or intended splice site.

In one example, a TfR-binding delivery domain coding sequence in a construct disclosed herein has one or more cryptic splice sites mutated or removed. In another example, a TfR-binding delivery domain coding sequence in a construct disclosed herein has all identified cryptic splice sites mutated or removed. In another example, a TfR-binding delivery domain coding sequence in a construct disclosed herein has one or more CpG dinucleotides removed (i.e., is CpG depleted). In another example, a TfR-binding delivery domain coding sequence in a construct disclosed herein has all CpG dinucleotides removed (i.e., is fully CpG depleted). In another example, a TfR-binding delivery domain coding sequence in a construct disclosed herein is codon optimized (e.g., codon optimized for expression in a human or mammal). In a specific example, a CDTfR63-binding delivery domain coding sequence in a construct disclosed herein has one or more CpG dinucleotides removed (i.e., is CpG depleted) and has one or more cryptic splice sites mutated or removed. In another specific example, a TfR-binding delivery domain coding sequence in a construct disclosed herein has all CpG dinucleotides removed and has one or more or all identified cryptic splice sites mutated or removed. In another specific example, a TfR-binding delivery domain coding sequence in a construct disclosed herein has one or more CpG dinucleotides removed (i.e., is CpG depleted) and is codon optimized (e.g., codon optimized for expression in a human or mammal). In another specific example, a TfR-binding delivery domain coding sequence in a construct disclosed herein has all CpG dinucleotides removed (i.e., is fully CpG depleted) and is codon optimized (e.g., codon optimized for expression in a human or mammal).

Various anti-TfR scFv coding sequences are provided. In one example, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 538-569 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to any one of SEQ ID NOS: 538-569 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to any one of SEQ ID NOS: 538-569 (and, e.g., retaining TfR-binding activity). Optionally, the anti-TfR scFv coding sequence in the above examples encodes an anti-TfR scFv protein comprising the sequence set forth in any one of SEQ ID NOS: 538-569. Optionally, the anti-TfR scFv coding sequence in the above examples encodes an anti-TfR scFv protein consisting essentially of the sequence set forth in any one of SEQ ID NOS: 538-569. Optionally, the anti-TfR scFv coding sequence in the above examples encodes an anti-TfR scFv protein consisting of the sequence set forth in any one of SEQ ID NOS: 538-569.

Various anti-TfR scFv coding sequences are provided. In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 587-599. In another example, the anti-TfR scFv coding sequence is (or comprises a sequence) at least 95%, at least 96%, at least 97%, at least 98%, at least 99%, at least 99.5%, or 100% identical to any one of SEQ ID NOS: 587-599. In another example, the anti-TfR scFv coding sequence is (or comprises a sequence) at least 99%, at least 99.5%, or 100% identical to any one of SEQ ID NOS: 587-599. In another example, the anti-TfR scFv coding sequence comprises the sequence set forth in any one of SEQ ID NOS: 587-599. In another example, the anti-TfR scFv coding sequence consists essentially of the sequence set forth in any one of SEQ ID NOS: 587-599. In another example, the anti-TfR scFv coding sequence consists of the sequence set forth in any one of SEQ ID NOS: 587-599. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 540, 549, 551, and 554 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to any one of SEQ ID NOS: 540, 549, 551, and 554 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to any one of SEQ ID NOS: 540, 549, 551, and 554 (and, e.g., retaining TfR-binding activity). Optionally, the anti-TfR scFv coding sequence in the above examples encodes an anti-TfR scFv protein comprising the sequence set forth in any one of SEQ ID NOS: 540, 549, 551, and 554. Optionally, the anti-TfR scFv coding sequence in the above examples encodes an anti-TfR scFv protein consisting essentially of the sequence set forth in any one of SEQ ID NOS: 540, 549, 551, and 554. Optionally, the anti-TfR scFv coding sequence in the above examples encodes an anti-TfR scFv protein consisting of the sequence set forth in any one of SEQ ID NOS: 540, 549, 551, and 554.

Various anti-TfR scFv coding sequences are provided. In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 593-595 and 599. In another example, the anti-TfR scFv coding sequence is (or comprises a sequence) at least 95%, at least 96%, at least 97%, at least 98%, at least 99%, at least 99.5%, or 100% identical to any one of SEQ ID NOS: 593-595 and 599. In another example, the anti-TfR scFv coding sequence is (or comprises a sequence) at least 99%, at least 99.5%, or 100% identical to any one of SEQ ID NOS: 593-595 and 599. In another example, the anti-TfR scFv coding sequence comprises the sequence set forth in any one of SEQ ID NOS: 593-595 and 599. In another example, the anti-TfR scFv coding sequence consists essentially of the sequence set forth in any one of SEQ ID NOS: 593-595 and 599. In another example, the anti-TfR scFv coding sequence consists of the sequence set forth in any one of SEQ ID NOS: 593-595 and 599. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 554 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 554 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 554 (and, e.g., retaining TfR-binding activity). Optionally, the anti-TfR scFv coding sequence in the above examples encodes an anti-TfR scFv protein comprising the sequence set forth in SEQ ID NO: 554. Optionally, the anti-TfR scFv coding sequence in the above examples encodes an anti-TfR scFv protein consisting essentially of the sequence set forth in SEQ ID NO: 554. Optionally, the anti-TfR scFv coding sequence in the above examples encodes an anti-TfR scFv protein consisting of the sequence set forth in SEQ ID NO: 554.

Various codon optimized anti-TfR scFv coding sequences are provided. The anti-TfR scFv coding sequence can be, for example, CpG-depleted (e.g., fully CpG depleted) and/or codon optimized (e.g., CpG depleted (e.g., fully CpG-depleted) and codon optimized). In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 587-595. In another example, the anti-TfR scFv coding sequence is (or comprises a sequence) at least 95%, at least 96%, at least 97%, at least 98%, at least 99%, at least 99.5%, or 100% identical to any one of SEQ ID NOS: 587-595. In another example, the anti-TfR scFv coding sequence is (or comprises a sequence) at least 99%, at least 99.5%, or 100% identical to any one of SEQ ID NOS: 587-595. In another example, the anti-TfR scFv coding sequence comprises the sequence set forth in any one of SEQ ID NOS: 587-595. In another example, the anti-TfR scFv coding sequence consists essentially of the sequence set forth in any one of SEQ ID NOS: 587-595. In another example, the anti-TfR scFv coding sequence consists of the sequence set forth in any one of SEQ ID NOS: 587-595. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 540, 549, 551, and 554 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to any one of SEQ ID NOS: 540, 549, 551, and 554 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to any one of SEQ ID NOS: 540, 549, 551, and 554 (and, e.g., retaining TfR-binding activity). Optionally, the anti-TfR scFv coding sequence in the above examples encodes an anti-TfR scFv protein comprising the sequence set forth in any one of SEQ ID NOS: 540, 549, 551, and 554. Optionally, the anti-TfR scFv coding sequence in the above examples encodes an anti-TfR scFv protein consisting essentially of the sequence set forth in any one of SEQ ID NOS: 540, 549, 551, and 554. Optionally, the anti-TfR scFv coding sequence in the above examples encodes an anti-TfR scFv protein consisting of the sequence set forth in any one of SEQ ID NOS: 540, 549, 551, and 554.

Various codon optimized anti-TfR scFv coding sequences are provided. The anti-TfR scFv coding sequence can be, for example, CpG-depleted (e.g., fully CpG depleted) and/or codon optimized (e.g., CpG depleted (e.g., fully CpG-depleted) and codon optimized). In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 593-595. In another example, the anti-TfR scFv coding sequence is (or comprises a sequence) at least 95%, at least 96%, at least 97%, at least 98%, at least 99%, at least 99.5%, or 100% identical to any one of SEQ ID NOS: 593-595. In another example, the anti-TfR scFv coding sequence is (or comprises a sequence) at least 99%, at least 99.5%, or 100% identical to any one of SEQ ID NOS: 593-595. In another example, the anti-TfR scFv coding sequence comprises the sequence set forth in any one of SEQ ID NOS: 593-595. In another example, the anti-TfR scFv coding sequence consists essentially of the sequence set forth in any one of SEQ ID NOS: 593-595. In another example, the anti-TfR scFv coding sequence consists of the sequence set forth in any one of SEQ ID NOS: 593-595. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 554 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 554 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 554 (and, e.g., retaining TfR-binding activity). Optionally, the anti-TfR scFv coding sequence in the above examples encodes an anti-TfR scFv protein comprising the sequence set forth in SEQ ID NO: 554. Optionally, the anti-TfR scFv coding sequence in the above examples encodes an anti-TfR scFv protein consisting essentially of the sequence set forth in SEQ ID NO: 554. Optionally, the anti-TfR scFv coding sequence in the above examples encodes an anti-TfR scFv protein consisting of the sequence set forth in SEQ ID NO: 554.

In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 593. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 593 and encodes an anti-TfR scFv protein (or an anti-TfR scFv protein comprising a sequence) at least 99%, at least 99.5%, or 100% identical to SEQ ID NO: 554. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 593 and encodes an anti-TfR scFv protein comprising the sequence set forth in SEQ ID NO: 554. In another example, the anti-TfR scFv coding sequence is (or comprises a sequence) at least 95%, at least 96%, at least 97%, at least 98%, at least 99%, at least 99.5%, or 100% identical to SEQ ID NO: 593. In another example, the anti-TfR scFv coding sequence is (or comprises a sequence) at least 95%, at least 96%, at least 97%, at least 98%, at least 99%, at least 99.5%, or 100% identical to SEQ ID NO: 593 and encodes an anti-TfR scFv protein (or an anti-TfR scFv protein comprising a sequence) at least 99%, at least 99.5%, or 100% identical to SEQ ID NO: 554. In another example, the anti-TfR scFv coding sequence is (or comprises a sequence) at least 95%, at least 96%, at least 97%, at least 98%, at least 99%, at least 99.5%, or 100% identical to SEQ ID NO: 593 and encodes an anti-TfR scFv protein comprising the sequence set forth in SEQ ID NO: 554. In another example, the anti-TfR scFv coding sequence is (or comprises a sequence) at least 99%, at least 99.5%, or 100% identical to SEQ ID NO: 593. In another example, the anti-TfR scFv coding sequence is (or comprises a sequence) at least 99%, at least 99.5%, or 100% identical to SEQ ID NO: 593 and encodes an anti-TfR scFv protein (or an anti-TfR scFv protein comprising a sequence) at least 99%, at least 99.5%, or 100% identical to SEQ ID NO: 554. In another example, the anti-TfR scFv coding sequence is (or comprises a sequence) at least 99%, at least 99.5%, or 100% identical to SEQ ID NO: 593 and encodes an anti-TfR scFv protein comprising the sequence set forth in SEQ ID NO: 554. In another example, the anti-TfR scFv coding sequence comprises the sequence set forth in SEQ ID NO: 593. In another example, the anti-TfR scFv coding sequence consists essentially of the sequence set forth in SEQ ID NO: 593. In another example, the anti-TfR scFv coding sequence consists of the sequence set forth in SEQ ID NO: 593. The anti-TfR coding sequence can be, for example, CpG-depleted (e.g., fully CpG-depleted) and/or codon optimized. For example, the anti-TfR scFv coding sequence can be CpG depleted (e.g., fully CpG-depleted) and codon optimized. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 554 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 554 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 554 (and, e.g., retaining TfR-binding activity). Optionally, the anti-TfR scFv coding sequence in the above examples encodes an anti-TfR scFv protein comprising the sequence set forth in SEQ ID NO: 554. Optionally, the anti-TfR scFv coding sequence in the above examples encodes an anti-TfR scFv protein consisting essentially of the sequence set forth in SEQ ID NO: 554. Optionally, the anti-TfR scFv coding sequence in the above examples encodes an anti-TfR scFv protein consisting of the sequence set forth in SEQ ID NO: 554.

In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 594. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 594 and encodes an anti-TfR scFv protein (or an anti-TfR scFv protein comprising a sequence) at least 99%, at least 99.5%, or 100% identical to SEQ ID NO: 554. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 594 and encodes an anti-TfR scFv protein comprising the sequence set forth in SEQ ID NO: 554. In another example, the anti-TfR scFv coding sequence is (or comprises a sequence) at least 95%, at least 96%, at least 97%, at least 98%, at least 99%, at least 99.5%, or 100% identical to SEQ ID NO: 594. In another example, the anti-TfR scFv coding sequence is (or comprises a sequence) at least 95%, at least 96%, at least 97%, at least 98%, at least 99%, at least 99.5%, or 100% identical to SEQ ID NO: 594 and encodes an anti-TfR scFv protein (or an anti-TfR scFv protein comprising a sequence) at least 99%, at least 99.5%, or 100% identical to SEQ ID NO: 554. In another example, the anti-TfR scFv coding sequence is (or comprises a sequence) at least 95%, at least 96%, at least 97%, at least 98%, at least 99%, at least 99.5%, or 100% identical to SEQ ID NO: 594 and encodes an anti-TfR scFv protein comprising the sequence set forth in SEQ ID NO: 554. In another example, the anti-TfR scFv coding sequence is (or comprises a sequence) at least 99%, at least 99.5%, or 100% identical to SEQ ID NO: 594. In another example, the anti-TfR scFv coding sequence is (or comprises a sequence) at least 99%, at least 99.5%, or 100% identical to SEQ ID NO: 594 and encodes an anti-TfR scFv protein (or an anti-TfR scFv protein comprising a sequence) at least 99%, at least 99.5%, or 100% identical to SEQ ID NO: 554. In another example, the anti-TfR scFv coding sequence is (or comprises a sequence) at least 99%, at least 99.5%, or 100% identical to SEQ ID NO: 594 and encodes an anti-TfR scFv protein comprising the sequence set forth in SEQ ID NO: 554. In another example, the anti-TfR scFv coding sequence comprises the sequence set forth in SEQ ID NO: 594. In another example, the anti-TfR scFv coding sequence consists essentially of the sequence set forth in SEQ ID NO: 594. In another example, the anti-TfR scFv coding sequence consists of the sequence set forth in SEQ ID NO: 594. The anti-TfR coding sequence can be, for example, CpG-depleted (e.g., fully CpG-depleted) and/or codon optimized. For example, the anti-TfR scFv coding sequence can be CpG depleted (e.g., fully CpG-depleted) and codon optimized. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 554 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 554 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 554 (and, e.g., retaining TfR-binding activity). Optionally, the anti-TfR scFv coding sequence in the above examples encodes an anti-TfR scFv protein comprising the sequence set forth in SEQ ID NO: 554. Optionally, the anti-TfR scFv coding sequence in the above examples encodes an anti-TfR scFv protein consisting essentially of the sequence set forth in SEQ ID NO: 554. Optionally, the anti-TfR scFv coding sequence in the above examples encodes an anti-TfR scFv protein consisting of the sequence set forth in SEQ ID NO: 554.

In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 595. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 595 and encodes an anti-TfR scFv protein (or an anti-TfR scFv protein comprising a sequence) at least 99%, at least 99.5%, or 100% identical to SEQ ID NO: 554. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 595 and encodes an anti-TfR scFv protein comprising the sequence set forth in SEQ ID NO: 554. In another example, the anti-TfR scFv coding sequence is (or comprises a sequence) at least 95%, at least 96%, at least 97%, at least 98%, at least 99%, at least 99.5%, or 100% identical to SEQ ID NO: 595. In another example, the anti-TfR scFv coding sequence is (or comprises a sequence) at least 95%, at least 96%, at least 97%, at least 98%, at least 99%, at least 99.5%, or 100% identical to SEQ ID NO: 595 and encodes an anti-TfR scFv protein (or an anti-TfR scFv protein comprising a sequence) at least 99%, at least 99.5%, or 100% identical to SEQ ID NO: 554. In another example, the anti-TfR scFv coding sequence is (or comprises a sequence) at least 95%, at least 96%, at least 97%, at least 98%, at least 99%, at least 99.5%, or 100% identical to SEQ ID NO: 595 and encodes an anti-TfR scFv protein comprising the sequence set forth in SEQ ID NO: 554. In another example, the anti-TfR scFv coding sequence is (or comprises a sequence) at least 99%, at least 99.5%, or 100% identical to SEQ ID NO: 595. In another example, the anti-TfR scFv coding sequence is (or comprises a sequence) at least 99%, at least 99.5%, or 100% identical to SEQ ID NO: 595 and encodes an anti-TfR scFv protein (or an anti-TfR scFv protein comprising a sequence) at least 99%, at least 99.5%, or 100% identical to SEQ ID NO: 554. In another example, the anti-TfR scFv coding sequence is (or comprises a sequence) at least 99%, at least 99.5%, or 100% identical to SEQ ID NO: 595 and encodes an anti-TfR scFv protein comprising the sequence set forth in SEQ ID NO: 554. In another example, the anti-TfR scFv coding sequence comprises the sequence set forth in SEQ ID NO: 595. In another example, the anti-TfR scFv coding sequence consists essentially of the sequence set forth in SEQ ID NO: 595. In another example, the anti-TfR scFv coding sequence consists of the sequence set forth in SEQ ID NO: 595. The anti-TfR coding sequence can be, for example, CpG-depleted (e.g., fully CpG-depleted) and/or codon optimized. For example, the anti-TfR scFv coding sequence can be CpG depleted (e.g., fully CpG-depleted) and codon optimized. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 554 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 554 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 554 (and, e.g., retaining TfR-binding activity). Optionally, the anti-TfR scFv coding sequence in the above examples encodes an anti-TfR scFv protein comprising the sequence set forth in SEQ ID NO: 554. Optionally, the anti-TfR scFv coding sequence in the above examples encodes an anti-TfR scFv protein consisting essentially of the sequence set forth in SEQ ID NO: 554. Optionally, the anti-TfR scFv coding sequence in the above examples encodes an anti-TfR scFv protein consisting of the sequence set forth in SEQ ID NO: 554.

Various codon optimized anti-TfR scFv coding sequences are provided. The anti-TfR scFv coding sequence can be, for example, CpG-depleted (e.g., fully CpG depleted) and/or codon optimized (e.g., CpG depleted (e.g., fully CpG-depleted) and codon optimized). In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 590-592. In another example, the anti-TfR scFv coding sequence is (or comprises a sequence) at least 95%, at least 96%, at least 97%, at least 98%, at least 99%, at least 99.5%, or 100% identical to any one of SEQ ID NOS: 590-592. In another example, the anti-TfR scFv coding sequence is (or comprises a sequence) at least 99%, at least 99.5%, or 100% identical to any one of SEQ ID NOS: 590-592. In another example, the anti-TfR scFv coding sequence comprises the sequence set forth in any one of SEQ ID NOS: 590-592. In another example, the anti-TfR scFv coding sequence consists essentially of the sequence set forth in any one of SEQ ID NOS: 590-592. In another example, the anti-TfR scFv coding sequence consists of the sequence set forth in any one of SEQ ID NOS: 590-592. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 551 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 551 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 551 (and, e.g., retaining TfR-binding activity). Optionally, the anti-TfR scFv coding sequence in the above examples encodes an anti-TfR scFv protein comprising the sequence set forth in SEQ ID NO: 551. Optionally, the anti-TfR scFv coding sequence in the above examples encodes an anti-TfR scFv protein consisting essentially of the sequence set forth in SEQ ID NO: 551. Optionally, the anti-TfR scFv coding sequence in the above examples encodes an anti-TfR scFv protein consisting of the sequence set forth in SEQ ID NO: 551.

In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 590. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 590 and encodes an anti-TfR scFv protein (or an anti-TfR scFv protein comprising a sequence) at least 99%, at least 99.5%, or 100% identical to SEQ ID NO: 551. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 590 and encodes an anti-TfR scFv protein comprising the sequence set forth in SEQ ID NO: 551. In another example, the anti-TfR scFv coding sequence is (or comprises a sequence) at least 95%, at least 96%, at least 97%, at least 98%, at least 99%, at least 99.5%, or 100% identical to SEQ ID NO: 590. In another example, the anti-TfR scFv coding sequence is (or comprises a sequence) at least 95%, at least 96%, at least 97%, at least 98%, at least 99%, at least 99.5%, or 100% identical to SEQ ID NO: 590 and encodes an anti-TfR scFv protein (or an anti-TfR scFv protein comprising a sequence) at least 99%, at least 99.5%, or 100% identical to SEQ ID NO: 551. In another example, the anti-TfR scFv coding sequence is (or comprises a sequence) at least 95%, at least 96%, at least 97%, at least 98%, at least 99%, at least 99.5%, or 100% identical to SEQ ID NO: 590 and encodes an anti-TfR scFv protein comprising the sequence set forth in SEQ ID NO: 551. In another example, the anti-TfR scFv coding sequence is (or comprises a sequence) at least 99%, at least 99.5%, or 100% identical to SEQ ID NO: 590. In another example, the anti-TfR scFv coding sequence is (or comprises a sequence) at least 99%, at least 99.5%, or 100% identical to SEQ ID NO: 590 and encodes an anti-TfR scFv protein (or an anti-TfR scFv protein comprising a sequence) at least 99%, at least 99.5%, or 100% identical to SEQ ID NO: 551. In another example, the anti-TfR scFv coding sequence is (or comprises a sequence) at least 99%, at least 99.5%, or 100% identical to SEQ ID NO: 590 and encodes an anti-TfR scFv protein comprising the sequence set forth in SEQ ID NO: 551. In another example, the anti-TfR scFv coding sequence comprises the sequence set forth in SEQ ID NO: 590. In another example, the anti-TfR scFv coding sequence consists essentially of the sequence set forth in SEQ ID NO: 590. In another example, the anti-TfR scFv coding sequence consists of the sequence set forth in SEQ ID NO: 590. The anti-TfR coding sequence can be, for example, CpG-depleted (e.g., fully CpG-depleted) and/or codon optimized. For example, the anti-TfR scFv coding sequence can be CpG depleted (e.g., fully CpG-depleted) and codon optimized. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 551 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 551 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 551 (and, e.g., retaining TfR-binding activity). Optionally, the anti-TfR scFv coding sequence in the above examples encodes an anti-TfR scFv protein comprising the sequence set forth in SEQ ID NO: 551. Optionally, the anti-TfR scFv coding sequence in the above examples encodes an anti-TfR scFv protein consisting essentially of the sequence set forth in SEQ ID NO: 551. Optionally, the anti-TfR scFv coding sequence in the above examples encodes an anti-TfR scFv protein consisting of the sequence set forth in SEQ ID NO: 551.

In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 591. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 591 and encodes an anti-TfR scFv protein (or an anti-TfR scFv protein comprising a sequence) at least 99%, at least 99.5%, or 100% identical to SEQ ID NO: 551. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 591 and encodes an anti-TfR scFv protein comprising the sequence set forth in SEQ ID NO: 551. In another example, the anti-TfR scFv coding sequence is (or comprises a sequence) at least 95%, at least 96%, at least 97%, at least 98%, at least 99%, at least 99.5%, or 100% identical to SEQ ID NO: 591. In another example, the anti-TfR scFv coding sequence is (or comprises a sequence) at least 95%, at least 96%, at least 97%, at least 98%, at least 99%, at least 99.5%, or 100% identical to SEQ ID NO: 591 and encodes an anti-TfR scFv protein (or an anti-TfR scFv protein comprising a sequence) at least 99%, at least 99.5%, or 100% identical to SEQ ID NO: 551. In another example, the anti-TfR scFv coding sequence is (or comprises a sequence) at least 95%, at least 96%, at least 97%, at least 98%, at least 99%, at least 99.5%, or 100% identical to SEQ ID NO: 591 and encodes an anti-TfR scFv protein comprising the sequence set forth in SEQ ID NO: 551. In another example, the anti-TfR scFv coding sequence is (or comprises a sequence) at least 99%, at least 99.5%, or 100% identical to SEQ ID NO: 591. In another example, the anti-TfR scFv coding sequence is (or comprises a sequence) at least 99%, at least 99.5%, or 100% identical to SEQ ID NO: 591 and encodes an anti-TfR scFv protein (or an anti-TfR scFv protein comprising a sequence) at least 99%, at least 99.5%, or 100% identical to SEQ ID NO: 551. In another example, the anti-TfR scFv coding sequence is (or comprises a sequence) at least 99%, at least 99.5%, or 100% identical to SEQ ID NO: 591 and encodes an anti-TfR scFv protein comprising the sequence set forth in SEQ ID NO: 551. In another example, the anti-TfR scFv coding sequence comprises the sequence set forth in SEQ ID NO: 591. In another example, the anti-TfR scFv coding sequence consists essentially of the sequence set forth in SEQ ID NO: 591. In another example, the anti-TfR scFv coding sequence consists of the sequence set forth in SEQ ID NO: 591. The anti-TfR coding sequence can be, for example, CpG-depleted (e.g., fully CpG-depleted) and/or codon optimized. For example, the anti-TfR scFv coding sequence can be CpG depleted (e.g., fully CpG-depleted) and codon optimized. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 551 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 551 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 551 (and, e.g., retaining TfR-binding activity). Optionally, the anti-TfR scFv coding sequence in the above examples encodes an anti-TfR scFv protein comprising the sequence set forth in SEQ ID NO: 551. Optionally, the anti-TfR scFv coding sequence in the above examples encodes an anti-TfR scFv protein consisting essentially of the sequence set forth in SEQ ID NO: 551. Optionally, the anti-TfR scFv coding sequence in the above examples encodes an anti-TfR scFv protein consisting of the sequence set forth in SEQ ID NO: 551.

In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 592. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 592 and encodes an anti-TfR scFv protein (or an anti-TfR scFv protein comprising a sequence) at least 99%, at least 99.5%, or 100% identical to SEQ ID NO: 551. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 592 and encodes an anti-TfR scFv protein comprising the sequence set forth in SEQ ID NO: 551. In another example, the anti-TfR scFv coding sequence is (or comprises a sequence) at least 95%, at least 96%, at least 97%, at least 98%, at least 99%, at least 99.5%, or 100% identical to SEQ ID NO: 592. In another example, the anti-TfR scFv coding sequence is (or comprises a sequence) at least 95%, at least 96%, at least 97%, at least 98%, at least 99%, at least 99.5%, or 100% identical to SEQ ID NO: 592 and encodes an anti-TfR scFv protein (or an anti-TfR scFv protein comprising a sequence) at least 99%, at least 99.5%, or 100% identical to SEQ ID NO: 551. In another example, the anti-TfR scFv coding sequence is (or comprises a sequence) at least 95%, at least 96%, at least 97%, at least 98%, at least 99%, at least 99.5%, or 100% identical to SEQ ID NO: 592 and encodes an anti-TfR scFv protein comprising the sequence set forth in SEQ ID NO: 551. In another example, the anti-TfR scFv coding sequence is (or comprises a sequence) at least 99%, at least 99.5%, or 100% identical to SEQ ID NO: 592. In another example, the anti-TfR scFv coding sequence is (or comprises a sequence) at least 99%, at least 99.5%, or 100% identical to SEQ ID NO: 592 and encodes an anti-TfR scFv protein (or an anti-TfR scFv protein comprising a sequence) at least 99%, at least 99.5%, or 100% identical to SEQ ID NO: 551. In another example, the anti-TfR scFv coding sequence is (or comprises a sequence) at least 99%, at least 99.5%, or 100% identical to SEQ ID NO: 592 and encodes an anti-TfR scFv protein comprising the sequence set forth in SEQ ID NO: 551. In another example, the anti-TfR scFv coding sequence comprises the sequence set forth in SEQ ID NO: 592. In another example, the anti-TfR scFv coding sequence consists essentially of the sequence set forth in SEQ ID NO: 592. In another example, the anti-TfR scFv coding sequence consists of the sequence set forth in SEQ ID NO: 592. The anti-TfR coding sequence can be, for example, CpG-depleted (e.g., fully CpG-depleted) and/or codon optimized. For example, the anti-TfR scFv coding sequence can be CpG depleted (e.g., fully CpG-depleted) and codon optimized. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 551 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 551 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 551 (and, e.g., retaining TfR-binding activity). Optionally, the anti-TfR scFv coding sequence in the above examples encodes an anti-TfR scFv protein comprising the sequence set forth in SEQ ID NO: 551. Optionally, the anti-TfR scFv coding sequence in the above examples encodes an anti-TfR scFv protein consisting essentially of the sequence set forth in SEQ ID NO: 551. Optionally, the anti-TfR scFv coding sequence in the above examples encodes an anti-TfR scFv protein consisting of the sequence set forth in SEQ ID NO: 551.

Various codon optimized anti-TfR scFv coding sequences are provided. The anti-TfR scFv coding sequence can be, for example, CpG-depleted (e.g., fully CpG depleted) and/or codon optimized (e.g., CpG depleted (e.g., fully CpG-depleted) and codon optimized). In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 587-589. In another example, the anti-TfR scFv coding sequence is (or comprises a sequence) at least 95%, at least 96%, at least 97%, at least 98%, at least 99%, at least 99.5%, or 100% identical to any one of SEQ ID NOS: 587-589. In another example, the anti-TfR scFv coding sequence is (or comprises a sequence) at least 99%, at least 99.5%, or 100% identical to any one of SEQ ID NOS: 587-589. In another example, the anti-TfR scFv coding sequence comprises the sequence set forth in any one of SEQ ID NOS: 587-589. In another example, the anti-TfR scFv coding sequence consists essentially of the sequence set forth in any one of SEQ ID NOS: 587-589. In another example, the anti-TfR scFv coding sequence consists of the sequence set forth in any one of SEQ ID NOS: 587-589. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 540 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 540 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 540 (and, e.g., retaining TfR-binding activity). Optionally, the anti-TfR scFv coding sequence in the above examples encodes an anti-TfR scFv protein comprising the sequence set forth in SEQ ID NO: 540. Optionally, the anti-TfR scFv coding sequence in the above examples encodes an anti-TfR scFv protein consisting essentially of the sequence set forth in SEQ ID NO: 540. Optionally, the anti-TfR scFv coding sequence in the above examples encodes an anti-TfR scFv protein consisting of the sequence set forth in SEQ ID NO: 540.

In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 587. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 587 and encodes an anti-TfR scFv protein (or an anti-TfR scFv protein comprising a sequence) at least 99%, at least 99.5%, or 100% identical to SEQ ID NO: 540. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 587 and encodes an anti-TfR scFv protein comprising the sequence set forth in SEQ ID NO: 540. In another example, the anti-TfR scFv coding sequence is (or comprises a sequence) at least 95%, at least 96%, at least 97%, at least 98%, at least 99%, at least 99.5%, or 100% identical to SEQ ID NO: 587. In another example, the anti-TfR scFv coding sequence is (or comprises a sequence) at least 95%, at least 96%, at least 97%, at least 98%, at least 99%, at least 99.5%, or 100% identical to SEQ ID NO: 587 and encodes an anti-TfR scFv protein (or an anti-TfR scFv protein comprising a sequence) at least 99%, at least 99.5%, or 100% identical to SEQ ID NO: 540. In another example, the anti-TfR scFv coding sequence is (or comprises a sequence) at least 95%, at least 96%, at least 97%, at least 98%, at least 99%, at least 99.5%, or 100% identical to SEQ ID NO: 587 and encodes an anti-TfR scFv protein comprising the sequence set forth in SEQ ID NO: 540. In another example, the anti-TfR scFv coding sequence is (or comprises a sequence) at least 99%, at least 99.5%, or 100% identical to SEQ ID NO: 587. In another example, the anti-TfR scFv coding sequence is (or comprises a sequence) at least 99%, at least 99.5%, or 100% identical to SEQ ID NO: 587 and encodes an anti-TfR scFv protein (or an anti-TfR scFv protein comprising a sequence) at least 99%, at least 99.5%, or 100% identical to SEQ ID NO: 540. In another example, the anti-TfR scFv coding sequence is (or comprises a sequence) at least 99%, at least 99.5%, or 100% identical to SEQ ID NO: 587 and encodes an anti-TfR scFv protein comprising the sequence set forth in SEQ ID NO: 540. In another example, the anti-TfR scFv coding sequence comprises the sequence set forth in SEQ ID NO: 587. In another example, the anti-TfR scFv coding sequence consists essentially of the sequence set forth in SEQ ID NO: 587. In another example, the anti-TfR scFv coding sequence consists of the sequence set forth in SEQ ID NO: 587. The anti-TfR coding sequence can be, for example, CpG-depleted (e.g., fully CpG-depleted) and/or codon optimized. For example, the anti-TfR scFv coding sequence can be CpG depleted (e.g., fully CpG-depleted) and codon optimized. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 540 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 540 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 540 (and, e.g., retaining TfR-binding activity). Optionally, the anti-TfR scFv coding sequence in the above examples encodes an anti-TfR scFv protein comprising the sequence set forth in SEQ ID NO: 540. Optionally, the anti-TfR scFv coding sequence in the above examples encodes an anti-TfR scFv protein consisting essentially of the sequence set forth in SEQ ID NO: 540. Optionally, the anti-TfR scFv coding sequence in the above examples encodes an anti-TfR scFv protein consisting of the sequence set forth in SEQ ID NO: 540.

In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 588. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 588 and encodes an anti-TfR scFv protein (or an anti-TfR scFv protein comprising a sequence) at least 99%, at least 99.5%, or 100% identical to SEQ ID NO: 540. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 588 and encodes an anti-TfR scFv protein comprising the sequence set forth in SEQ ID NO: 540. In another example, the anti-TfR scFv coding sequence is (or comprises a sequence) at least 95%, at least 96%, at least 97%, at least 98%, at least 99%, at least 99.5%, or 100% identical to SEQ ID NO: 588. In another example, the anti-TfR scFv coding sequence is (or comprises a sequence) at least 95%, at least 96%, at least 97%, at least 98%, at least 99%, at least 99.5%, or 100% identical to SEQ ID NO: 588 and encodes an anti-TfR scFv protein (or an anti-TfR scFv protein comprising a sequence) at least 99%, at least 99.5%, or 100% identical to SEQ ID NO: 540. In another example, the anti-TfR scFv coding sequence is (or comprises a sequence) at least 95%, at least 96%, at least 97%, at least 98%, at least 99%, at least 99.5%, or 100% identical to SEQ ID NO: 588 and encodes an anti-TfR scFv protein comprising the sequence set forth in SEQ ID NO: 540. In another example, the anti-TfR scFv coding sequence is (or comprises a sequence) at least 99%, at least 99.5%, or 100% identical to SEQ ID NO: 588. In another example, the anti-TfR scFv coding sequence is (or comprises a sequence) at least 99%, at least 99.5%, or 100% identical to SEQ ID NO: 588 and encodes an anti-TfR scFv protein (or an anti-TfR scFv protein comprising a sequence) at least 99%, at least 99.5%, or 100% identical to SEQ ID NO: 540. In another example, the anti-TfR scFv coding sequence is (or comprises a sequence) at least 99%, at least 99.5%, or 100% identical to SEQ ID NO: 588 and encodes an anti-TfR scFv protein comprising the sequence set forth in SEQ ID NO: 540. In another example, the anti-TfR scFv coding sequence comprises the sequence set forth in SEQ ID NO: 588. In another example, the anti-TfR scFv coding sequence consists essentially of the sequence set forth in SEQ ID NO: 588. In another example, the anti-TfR scFv coding sequence consists of the sequence set forth in SEQ ID NO: 588. The anti-TfR coding sequence can be, for example, CpG-depleted (e.g., fully CpG-depleted) and/or codon optimized. For example, the anti-TfR scFv coding sequence can be CpG depleted (e.g., fully CpG-depleted) and codon optimized. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 540 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 540 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 540 (and, e.g., retaining TfR-binding activity). Optionally, the anti-TfR scFv coding sequence in the above examples encodes an anti-TfR scFv protein comprising the sequence set forth in SEQ ID NO: 540. Optionally, the anti-TfR scFv coding sequence in the above examples encodes an anti-TfR scFv protein consisting essentially of the sequence set forth in SEQ ID NO: 540. Optionally, the anti-TfR scFv coding sequence in the above examples encodes an anti-TfR scFv protein consisting of the sequence set forth in SEQ ID NO: 540.

In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 589. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 589 and encodes an anti-TfR scFv protein (or an anti-TfR scFv protein comprising a sequence) at least 99%, at least 99.5%, or 100% identical to SEQ ID NO: 540. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 589 and encodes an anti-TfR scFv protein comprising the sequence set forth in SEQ ID NO: 540. In another example, the anti-TfR scFv coding sequence is (or comprises a sequence) at least 95%, at least 96%, at least 97%, at least 98%, at least 99%, at least 99.5%, or 100% identical to SEQ ID NO: 589. In another example, the anti-TfR scFv coding sequence is (or comprises a sequence) at least 95%, at least 96%, at least 97%, at least 98%, at least 99%, at least 99.5%, or 100% identical to SEQ ID NO: 589 and encodes an anti-TfR scFv protein (or an anti-TfR scFv protein comprising a sequence) at least 99%, at least 99.5%, or 100% identical to SEQ ID NO: 540. In another example, the anti-TfR scFv coding sequence is (or comprises a sequence) at least 95%, at least 96%, at least 97%, at least 98%, at least 99%, at least 99.5%, or 100% identical to SEQ ID NO: 589 and encodes an anti-TfR scFv protein comprising the sequence set forth in SEQ ID NO: 540. In another example, the anti-TfR scFv coding sequence is (or comprises a sequence) at least 99%, at least 99.5%, or 100% identical to SEQ ID NO: 589. In another example, the anti-TfR scFv coding sequence is (or comprises a sequence) at least 99%, at least 99.5%, or 100% identical to SEQ ID NO: 589 and encodes an anti-TfR scFv protein (or an anti-TfR scFv protein comprising a sequence) at least 99%, at least 99.5%, or 100% identical to SEQ ID NO: 540. In another example, the anti-TfR scFv coding sequence is (or comprises a sequence) at least 99%, at least 99.5%, or 100% identical to SEQ ID NO: 589 and encodes an anti-TfR scFv protein comprising the sequence set forth in SEQ ID NO: 540. In another example, the anti-TfR scFv coding sequence comprises the sequence set forth in SEQ ID NO: 589. In another example, the anti-TfR scFv coding sequence consists essentially of the sequence set forth in SEQ ID NO: 589. In another example, the anti-TfR scFv coding sequence consists of the sequence set forth in SEQ ID NO: 589. The anti-TfR coding sequence can be, for example, CpG-depleted (e.g., fully CpG-depleted) and/or codon optimized. For example, the anti-TfR scFv coding sequence can be CpG depleted (e.g., fully CpG-depleted) and codon optimized. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 540 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 540 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 540 (and, e.g., retaining TfR-binding activity). Optionally, the anti-TfR scFv coding sequence in the above examples encodes an anti-TfR scFv protein comprising the sequence set forth in SEQ ID NO: 540. Optionally, the anti-TfR scFv coding sequence in the above examples encodes an anti-TfR scFv protein consisting essentially of the sequence set forth in SEQ ID NO: 540. Optionally, the anti-TfR scFv coding sequence in the above examples encodes an anti-TfR scFv protein consisting of the sequence set forth in SEQ ID NO: 540.

When specific anti-TfR scFv or multidomain therapeutic protein nucleic acid constructs sequences are disclosed herein, they are meant to encompass the sequence disclosed or the reverse complement of the sequence. For example, if an anti-TfR scFv or multidomain therapeutic protein nucleic acid construct disclosed herein consists of the hypothetical sequence 5′-CTGGACCGA-3′, it is also meant to encompass the reverse complement of that sequence (5′-TCGGTCCAG-3′). Likewise, when construct elements are disclosed herein in a specific 5′ to 3′ order, they are also meant to encompass the reverse complement of the order of those elements. One reason for this is that, in many embodiments disclosed herein, the anti-TfR scFv or multidomain therapeutic protein nucleic acid constructs are part of a single-stranded recombinant AAV vector. Single-stranded AAV genomes are packaged as either sense (plus-stranded) or anti-sense (minus-stranded genomes), and single-stranded AAV genomes of + and -polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

### Bidirectional Constructs

The nucleic acid constructs disclosed herein can be bidirectional constructs. Such bidirectional constructs can allow for enhanced insertion and expression of encoded polypeptide of interest. When used in combination with a nuclease agent (e.g., CRISPR/Cas system, zinc finger nuclease (ZFN) system; transcription activator-like effector nuclease (TALEN) system) as described herein, the bidirectionality of the nucleic acid construct allows the construct to be inserted in either direction (i.e., is not limited to insertion in one direction) within a target genomic locus or a cleavage site or target insertion site, allowing the expression of the polypeptide of interest when inserted in either orientation, thereby enhancing expression efficiency.

A bidirectional construct as disclosed herein can comprise at least two nucleic acid segments, wherein a first segment comprises a first coding sequence for the polypeptide of interest, and a second segment comprises the reverse complement of a second coding sequence for the polypeptide of interest, or vice versa. However, other bidirectional constructs disclosed herein can comprise at least two nucleic acid segments, wherein the first segment comprises a coding sequence for a polypeptide of interest, and the second segment comprises the reverse complement of a coding sequence for another protein, or vice versa. A reverse complement refers to a sequence that is a complement sequence of a reference sequence, wherein the complement sequence is written in the reverse orientation. For example, for a hypothetical sequence 5′-CTGGACCGA-3′, the perfect complement sequence is 3′-GACCTGGCT-5′, and the perfect reverse complement is written 5′-TCGGTCCAG-3′. A reverse complement sequence need not be perfect and may still encode the same polypeptide or a similar polypeptide as the reference sequence. Due to codon usage redundancy, a reverse complement can diverge from a reference sequence that encodes the same polypeptide. The coding sequences can optionally comprise one or more additional sequences, such as sequences encoding amino- or carboxy-terminal amino acid sequences such as a signal sequence, label sequence (e.g., HiBit), or heterologous functional sequence (e.g., nuclear localization sequence (NLS) or self-cleaving peptide) linked to the polypeptide of interest or other protein.

When specific bidirectional construct sequences are disclosed herein, they are meant to encompass the sequence disclosed or the reverse complement of the sequence. For example, if a bidirectional construct disclosed herein consists of the hypothetical sequence 5′-CTGGACCGA-3′, it is also meant to encompass the reverse complement of that sequence (5′-TCGGTCCAG-3′). Likewise, when bidirectional construct elements are disclosed herein in a specific 5′ to 3′ order, they are also meant to encompass the reverse complement of the order of those elements. For example, if a bidirectional construct is disclosed herein that comprises from 5′ to 3′ a first splice acceptor, a first coding sequence, a first terminator, a reverse complement of a second terminator, a reverse complement of a second coding sequence, and a reverse complement of a second splice acceptor, it is also meant to encompass a construct comprising from 5′ to 3′ the second splice acceptor, the second coding sequence, the second terminator, a reverse complement of the first terminator, a reverse complement of the first coding sequence, and a reverse complement of the first splice acceptor. One reason for this is that, in many embodiments disclosed herein, the bidirectional constructs are part of a single-stranded recombinant AAV vector. Single-stranded AAV genomes are packaged as either sense (plus-stranded) or anti-sense (minus-stranded genomes), and single-stranded AAV genomes of + and -polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

When the at least two segments both encode a polypeptide of interest, the at least two segments can encode the same polypeptide of interest or different polypeptides of interest. The different polypeptides of interest can be at least about 90%, at least about 95%, at least about 96%, at least about 97%, at least about 98%, at least about 99%, or at least about 99.5% identical. For example, the first segment can encode a wild type polypeptide of interest or fragment thereof, and the second segment can encode a variant of the polypeptide of interest or fragment thereof, or vice versa. Alternatively, the first segment can encode a first variant polypeptide of interest, and the second segment can encode a second variant polypeptide of interest that is different from the first variant polypeptide of interest. Preferably, the two segments encode the same polypeptide of interest (i.e., 100% identical).

Even when the two segments encode the same polypeptide of interest, the coding sequence for the polypeptide of interest in the first segment can differ from the coding sequence for the polypeptide of interest in the second segment. In some bidirectional constructs, the codon usage in the first coding sequence is the same as the codon usage in the second coding sequence. In other bidirectional constructs, the second coding sequence adopts a different codon usage from the codon usage of the first coding sequence in order to reduce hairpin formation. One or both of the coding sequences can be codon-optimized for expression in a host cell. In some bidirectional constructs, only one of the coding sequences is codon-optimized. In some bidirectional constructs, the first coding sequence is codon-optimized. In some bidirectional constructs, the second coding sequence is codon-optimized. In some bidirectional constructs, both coding sequences are codon-optimized. For example, the second polypeptide of interest coding sequence can be codon optimized or may use one or more alternative codons for one or more amino acids of the same polypeptide of interest (i.e., same amino acid sequence) encoded by the polypeptide of interest coding sequence in the first segment. An alternative codon as used herein refers to variations in codon usage for a given amino acid, and may or may not be a preferred or optimized codon (codon optimized) for a given expression system. Preferred codon usage, or codons that are well-tolerated in a given system of expression are known.

In one example, the second segment comprises a reverse complement of a polypeptide of interest coding sequence that adopts different codon usage from that of the polypeptide of interest coding sequence in the first segment in order to reduce hairpin formation. Such a reverse complement forms base pairs with fewer than all nucleotides of the coding sequence in the first segment, yet it optionally encodes the same polypeptide. In one example, the reverse complement sequence in the second segment is not substantially complementary (e.g., not more than 70% complementary) to the coding sequence in the first segment. In other cases, however, the second segment comprises a reverse complement sequence that is highly complementary (e.g., at least 90% complementary) to the coding sequence in the first segment.

The second segment can have any percentage of complementarity to the first segment. For example, the second segment sequence can have at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 97%, or at least about 99% complementarity to the first segment. As another example, the second segment sequence can have less than about 30%, less than about 35%, less than about 40%, less than about 45%, less than about 50%, less than about 55%, less than about 60%, less than about 65%, less than about 70%, less than about 75%, less than about 80%, less than about 85%, less than about 90%, less than about 95%, less than about 97%, or less than about 99% complementarity to the first segment. The reverse complement of the second coding sequence can be, in some nucleic acid constructs, not substantially complementary (e.g., not more than 70% complementary) to the first coding sequence, not substantially complementary to a fragment of the first coding sequence, highly complementary (e.g., at least 90% complementary) to the first coding sequence, highly complementary to a fragment of the first coding sequence, about 50% to about 80% identical to the reverse complement of the first coding sequence, or about 60% to about 100% identical to the reverse complement of the first coding sequence.

The bidirectional constructs disclosed herein can be modified to include any suitable structural feature as needed for any particular use and/or that confers one or more desired function. For example, the bidirectional nucleic acid constructs disclosed herein need not comprise a homology arm and/or can be, for example, homology-independent donor constructs. Owing in part to the bidirectional function of the nucleic acid constructs, the bidirectional constructs can be inserted into a genomic locus in either direction as described herein to allow for efficient insertion and/or expression of the polypeptide of interest.

In some cases, the bidirectional nucleic acid construct does not comprise a promoter that drives the expression of the polypeptide of interest. For example, the expression of the polypeptide of interest can be driven by a promoter of the host cell (e.g., the endogenous ALB promoter when the transgene is integrated into a host cell’s ALB locus). In other cases, the bidirectional nucleic acid construct can comprise one or more promoters operably linked to the coding sequences for the polypeptide of interest. That is, although not required for expression, the constructs disclosed herein may also include transcriptional or translational regulatory sequences such as promoters, enhancers, insulators, internal ribosome entry sites, additional sequences encoding peptides, and/or polyadenylation signals. Some bidirectional constructs can comprise a promoter that drives expression of the first polypeptide of interest coding sequence and/or the reverse complement of a promoter that drives expression of the reverse complement of the second polypeptide of interest coding sequence.

The bidirectional constructs disclosed herein can be modified to include or exclude any suitable structural feature as needed for any particular use and/or that confers one or more desired functions. For example, some bidirectional nucleic acid constructs disclosed herein do not comprise a homology arm. Owing in part to the bidirectional function of the nucleic acid construct, the bidirectional construct can be inserted into a genomic locus in either direction (orientation) as described herein to allow for efficient insertion and/or expression of a polypeptide of interest.

The bidirectional constructs can, in some cases, comprise one or more (e.g., two) polyadenylation tail sequences or polyadenylation signal sequences. In some bidirectional constructs, the first segment can comprise a polyadenylation signal sequence. In some bidirectional constructs, the second segment can comprise a polyadenylation signal sequence. In some bidirectional constructs, the first segment can comprise a first polyadenylation signal sequence, and the second segment can comprise a second polyadenylation signal sequence (e.g., a reverse complement of a polyadenylation signal sequence). In some bidirectional constructs, the first segment can comprise a first polyadenylation signal sequence located 3′ of the first coding sequence. In some bidirectional constructs, the second segment can comprise a reverse complement of a second polyadenylation signal sequence located 5′ of the reverse complement of the second coding sequence. In some bidirectional constructs, the first segment can comprise a first polyadenylation signal sequence located 3′ of the first coding sequence, and the second segment can comprise a reverse complement of a second polyadenylation signal sequence located 5′ of the reverse complement of the second coding sequence. The first and second polyadenylation signal sequences can be the same or different. In one example, the first and second polyadenylation signals are different. In a specific example, the first polyadenylation signal is a simian virus 40 (SV40) late polyadenylation signal (or a variant thereof), and the second polyadenylation signal is a bovine growth hormone (BGH) polyadenylation signal (or a variant thereof), or vice versa. For example, one polyadenylation signal can be an SV40 polyadenylation signal, and the other polyadenylation signal can be a BGH polyadenylation signal. In a specific example, one polyadenylation signal can comprise, consist essentially of, or consist of SEQ ID NO: 161, and the other polyadenylation signal can comprise, consist essentially of, or consist of SEQ ID NO: 162.

In some bidirectional constructs, both the first segment and the second segment comprise a polyadenylation tail sequence. Methods of designing a suitable polyadenylation tail sequence are known. For example, in some bidirectional constructs, one or both of the first and second segment comprises a polyadenylation tail sequence and/or a polyadenylation signal sequence downstream of an open reading frame (i.e., a polyadenylation tail sequence and/or a polyadenylation signal sequence 3′ of a coding sequence, or a reverse complement of a polyadenylation tail sequence and/or a polyadenylation signal sequence 5′ of a reverse complement of a coding sequence). The polyadenylation tail sequence can be encoded, for example, as a “poly-A” stretch downstream of the polypeptide of interest coding sequence (or other protein coding sequence) in the first and/or second segment. A poly-A tail can comprise, for example, at least 20, 30, 40, 50, 60, 70, 80, 90, or 100 adenines, and optionally up to 300 adenines. In a specific example, the poly-A tail comprises 95, 96, 97, 98, 99, or 100 adenine nucleotides. Methods of designing a suitable polyadenylation tail sequence and/or polyadenylation signal sequence are well known. For example, the polyadenylation signal sequence AAUAAA is commonly used in mammalian systems, although variants such as UAUAAA or AU/GUAAA have been identified. See, e.g., Proudfoot (2011) Genes & Dev. 25(17):1770-82, herein incorporated by reference in its entirety for all purposes. In some bidirectional constructs, a single bidirectional terminator can be used to terminate RNA polymerase transcription in either the sense or the antisense direction (i.e., to terminate RNA polymerase transcription from both the first segment and the second segment). Examples of bidirectional terminators include the ARO4, TRP1, TRP4, ADH1, CYC1, GAL1, GAL7, and GAL10 terminators.

The bidirectional constructs can, in some cases, comprise one or more (e.g., two) splice acceptor sites. In some bidirectional constructs, the first segment can comprise a splice acceptor site. In some bidirectional constructs, the second segment can comprise a splice acceptor site. In some bidirectional constructs, the first segment can comprise a first splice acceptor site, and the second segment can comprise a second splice acceptor site (e.g., a reverse complement of a splice acceptor site). In some bidirectional constructs, the first segment comprises a first splice acceptor site located 5′ of the first coding sequence. In some bidirectional constructs, the second segment comprises a reverse complement of a second splice acceptor site located 3′ of the reverse complement of the second coding sequence. In some bidirectional constructs, the first segment comprises a first splice acceptor site located 5′ of the first coding sequence, and the second segment comprises a reverse complement of a second splice acceptor site located 3′ of the reverse complement of the second coding sequence. The first and second splice acceptor sites can be the same or different. In a specific example, both splice acceptors are mouse Alb exon 2 splice acceptors. In a specific example, both splice acceptors can comprise, consist essentially of, or consist of SEQ ID NO: 163.

A bidirectional construct may comprise a first coding sequence that encodes a first coding sequence linked to a splice acceptor and a reverse complement of a second coding sequence operably linked to the reverse complement of a splice acceptor. The bidirectional constructs disclosed herein can also comprise a splice acceptor site on either or both ends of the construct, or splice acceptor sites in both the first segment and the second segment (e.g., a splice acceptor site 5′ of a coding sequence, or a reverse complement of a splice acceptor 3′ of a reverse complement of a coding sequence). The splice acceptor site can, for example, comprise NAG or consist of NAG. In a specific example, the splice acceptor is an ALB splice acceptor (e.g., an ALB splice acceptor used in the splicing together of exons 1 and 2 of ALB (i.e., ALB exon 2 splice acceptor)). For example, such a splice acceptor can be derived from the human ALB gene. In another example, the splice acceptor can be derived from the mouse Alb gene (e.g., an ALB splice acceptor used in the splicing together of exons 1 and 2 of mouse Alb (i.e., mouse Alb exon 2 splice acceptor)). In another example, the splice acceptor is a splice acceptor from a gene encoding the polypeptide of interest. Additional suitable splice acceptor sites useful in eukaryotes, including artificial splice acceptors, are known. See, e.g., Shapiro et al. (1987) Nucleic Acids Res. 15:7155-7174 and Burset et al. (2001) Nucleic Acids Res. 29:255-259, each of which is herein incorporated by reference in its entirety for all purposes. The splice acceptors used in a bidirectional construct may be the same or different. In a specific example, both splice acceptors are mouse Alb exon 2 splice acceptors.

The bidirectional constructs can be circular or linear. For example, a bidirectional construct can be linear. The first and second segments can be joined in a linear manner through a linker sequence. For example, the 5′ end of the second segment that comprises a reverse complement sequence can be linked to the 3′ end of the first segment. Alternatively, the 5′ end of the first segment can be linked to the 3′ end of the second segment that comprises a reverse complement sequence. The linker can be any suitable length. For example, the linker can be between about 5 to about 2000 nucleotides in length. As an example, the linker sequence can be about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, about 100, about 150, about 200, about 250, about 300, about 500, about 1000, about 1500, about 2000, or more nucleotides in length. Other structural elements in addition to, or instead of, a linker sequence, can also be inserted between the first and second segments.

The bidirectional constructs disclosed herein can be DNA or RNA, single-stranded, double-stranded, or partially single-stranded and partially double-stranded. For example, the constructs can be single- or double-stranded DNA. In some embodiments, the nucleic acid can be modified (e.g., using nucleoside analogs), as described herein. In a specific example, the bidirectional construct is single-stranded (e.g., single-stranded DNA).

The bidirectional constructs disclosed herein can be modified on either or both ends to include one or more suitable structural features as needed and/or to confer one or more functional benefit. For example, structural modifications can vary depending on the method(s) used to deliver the constructs disclosed herein to a host cell (e.g., use of viral vector delivery or packaging into lipid nanoparticles for delivery). Such modifications include, for example, terminal structures such as inverted terminal repeats (ITR), hairpin, loops, and other structures such as toroids. For example, the constructs disclosed herein can comprise one, two, or three ITRs or can comprise no more than two ITRs. Various methods of structural modifications are known.

Similarly, one or both ends of the construct can be protected (e.g., from exonucleolytic degradation) by known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in its entirety for all purposes. Additional methods for protecting the constructs from degradation include, but are not limited to, addition of terminal amino group(s) and the use of modified internucleotide linkages such as, for example, phosphorothioates, phosphoramidates, and O-methyl ribose or deoxyribose residues.

As disclosed in more detail herein, the bidirectional constructs disclosed herein can be introduced into a cell as part of a vector having additional sequences such as, for example, replication origins, promoters, and genes encoding antibiotic resistance. The constructs can be introduced as a naked nucleic acid, can be introduced as a nucleic acid complexed with an agent such as a liposome, polymer, or poloxamer, or can be delivered by viral vectors (e.g., adenovirus, AAV, herpesvirus, retrovirus, lentivirus).

In an exemplary bidirectional construct, the second segment is located 3′ of the first segment, the first polypeptide of interest coding sequence and the second polypeptide of interest coding sequence both encode the same human polypeptide of interest, the second polypeptide of interest coding sequence adopts a different codon usage from the codon usage of the first polypeptide of interest coding sequence, the first segment comprises a first polyadenylation signal sequence located 3′ of the first polypeptide of interest coding sequence, the second segment comprises a reverse complement of a second polyadenylation signal sequence located 5′ of the reverse complement of the second polypeptide of interest coding sequence, the first segment comprises a first splice acceptor site located 5′ of the first polypeptide of interest coding sequence, the second segment comprises a reverse complement of a second splice acceptor site located 3′ of the reverse complement of the second polypeptide of interest coding sequence, the nucleic acid construct does not comprise a promoter that drives expression of the first polypeptide of interest or the second polypeptide of interest, and optionally the nucleic acid construct does not comprise a homology arm.

### Unidirectional Constructs

The nucleic acid constructs disclosed herein can be unidirectional constructs. When specific unidirectional construct sequences are disclosed herein, they are meant to encompass the sequence disclosed or the reverse complement of the sequence. For example, if a unidirectional construct disclosed herein consists of the hypothetical sequence 5′-CTGGACCGA-3′, it is also meant to encompass the reverse complement of that sequence (5′-TCGGTCCAG-3′). Likewise, when unidirectional construct elements are disclosed herein in a specific 5′ to 3′ order, they are also meant to encompass the reverse complement of the order of those elements. One reason for this is that, in many embodiments disclosed herein, the unidirectional constructs are part of a single-stranded recombinant AAV vector. Single-stranded AAV genomes are packaged as either sense (plus-stranded) or anti-sense (minus-stranded genomes), and single-stranded AAV genomes of + and - polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

In the unidirectional constructs, the coding sequence for the polypeptide of interest can be codon-optimized for expression in a host cell. For example, the coding sequence can be codon optimized or may use one or more alternative codons for one or more amino acids of the polypeptide of interest (i.e., same amino acid sequence). An alternative codon as used herein refers to variations in codon usage for a given amino acid, and may or may not be a preferred or optimized codon (codon optimized) for a given expression system. Preferred codon usage, or codons that are well-tolerated in a given system of expression, are known.

The unidirectional constructs disclosed herein can be modified to include any suitable structural feature as needed for any particular use and/or that confers one or more desired functions. For example, the unidirectional nucleic acid constructs disclosed herein need not comprise a homology arm and/or can be, for example, homology-independent donor constructs.

In some cases, the unidirectional nucleic acid construct does not comprise a promoter that drives the expression of polypeptide of interest. For example, the expression of the polypeptide of interest can be driven by a promoter of the host cell (e.g., the endogenous ALB promoter when the transgene is integrated into a host cell’s ALB locus). In other cases, the unidirectional nucleic acid construct can comprise one or more promoters operably linked to the coding sequence for the polypeptide of interest. That is, although not required for expression, the constructs disclosed herein may also include transcriptional or translational regulatory sequences such as promoters, enhancers, insulators, internal ribosome entry sites, additional sequences encoding peptides, and/or polyadenylation signals. Some unidirectional constructs can comprise a promoter that drives expression of the coding sequence for the polypeptide of interest.

The unidirectional constructs can, in some cases, comprise one or more polyadenylation tail sequences or polyadenylation signal sequences. Some unidirectional constructs can comprise a polyadenylation signal sequence located 3′ of the coding sequence for the polypeptide of interest. In a specific example, the polyadenylation signal is a simian virus 40 (SV40) late polyadenylation signal (or a variant thereof). In another specific example, the polyadenylation signal is a bovine growth hormone (BGH) polyadenylation signal (or a variant thereof). In another specific example, the polyadenylation signal is a BGH polyadenylation signal. For example, the polyadenylation signal can be an SV40 polyadenylation signal or a BGH polyadenylation signal. In a specific example, the polyadenylation signal can comprise, consist essentially of, or consist of SEQ ID NO: 161. In another specific example, the polyadenylation signal can comprise, consist essentially of, or consist of SEQ ID NO: 162.

Methods of designing a suitable polyadenylation tail sequence are known. For example, some unidirectional constructs comprise a polyadenylation tail sequence and/or a polyadenylation signal sequence downstream of an open reading frame (i.e., a polyadenylation tail sequence and/or a polyadenylation signal sequence 3′ of a coding sequence). The polyadenylation tail sequence can be encoded, for example, as a “poly-A” stretch downstream of the coding sequence for the polypeptide of interest (or other protein coding sequence) in the first and/or second segment. A poly-A tail can comprise, for example, at least 20, 30, 40, 50, 60, 70, 80, 90, or 100 adenines, and optionally up to 300 adenines. In a specific example, the poly-A tail comprises 95, 96, 97, 98, 99, or 100 adenine nucleotides. Methods of designing a suitable polyadenylation tail sequence and/or polyadenylation signal sequence are well known. For example, the polyadenylation signal sequence AAUAAA is commonly used in mammalian systems, although variants such as UAUAAA or AU/GUAAA have been identified. See, e.g., Proudfoot (2011) Genes & Dev. 25(17):1770-82, herein incorporated by reference in its entirety for all purposes.

The unidirectional constructs can, in some cases, comprise one or more splice acceptor sites. Some unidirectional constructs comprise a splice acceptor site located 5′ of the coding sequence for the polypeptide of interest. In a specific example, the splice acceptor is a mouse Alb exon 2 splice acceptor. In a specific example, the splice acceptor can comprise, consist essentially of, or consist of SEQ ID NO: 163.

The splice acceptor site can, for example, comprise NAG or consist of NAG. In a specific example, the splice acceptor is an ALB splice acceptor (e.g., an ALB splice acceptor used in the splicing together of exons 1 and 2 of ALB (i.e., ALB exon 2 splice acceptor)). For example, such a splice acceptor can be derived from the human ALB gene. In another example, the splice acceptor can be derived from the mouse Alb gene (e.g., an ALB splice acceptor used in the splicing together of exons 1 and 2 of mouse Alb (i.e., mouse Alb exon 2 splice acceptor)). In another example, the splice acceptor is a splice acceptor from the gene encoding the polypeptide of interest. Additional suitable splice acceptor sites useful in eukaryotes, including artificial splice acceptors, are known. See, e.g., Shapiro et al. (1987) Nucleic Acids Res. 15:7155-7174 and Burset et al. (2001) Nucleic Acids Res. 29:255-259, each of which is herein incorporated by reference in its entirety for all purposes.

The unidirectional constructs can be circular or linear. For example, a unidirectional construct can be linear.

The unidirectional constructs disclosed herein can be DNA or RNA, single-stranded, double-stranded, or partially single-stranded and partially double-stranded. For example, the constructs can be single- or double-stranded DNA. In some embodiments, the nucleic acid can be modified (e.g., using nucleoside analogs), as described herein. In a specific example, the unidirectional construct is single-stranded (e.g., single-stranded DNA).

The unidirectional constructs disclosed herein can be modified on either or both ends to include one or more suitable structural features as needed and/or to confer one or more functional benefit. For example, structural modifications can vary depending on the method(s) used to deliver the constructs disclosed herein to a host cell (e.g., use of viral vector delivery or packaging into lipid nanoparticles for delivery). Such modifications include, for example, terminal structures such as inverted terminal repeats (ITR), hairpin, loops, and other structures such as toroids. For example, the constructs disclosed herein can comprise one, two, or three ITRs or can comprise no more than two ITRs. Various methods of structural modifications are known.

Similarly, one or both ends of the construct can be protected (e.g., from exonucleolytic degradation) by known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in its entirety for all purposes. Additional methods for protecting the constructs from degradation include, but are not limited to, addition of terminal amino group(s) and the use of modified internucleotide linkages such as, for example, phosphorothioates, phosphoramidates, and O-methyl ribose or deoxyribose residues.

As disclosed in more detail herein, the unidirectional constructs disclosed herein can be introduced into a cell as part of a vector having additional sequences such as, for example, replication origins, promoters, and genes encoding antibiotic resistance. The constructs can be introduced as a naked nucleic acid, can be introduced as a nucleic acid complexed with an agent such as a liposome, polymer, or poloxamer, or can be delivered by viral vectors (e.g., adenovirus, AAV, herpesvirus, retrovirus, lentivirus).

In an exemplary unidirectional construct, the construct comprises a polyadenylation signal sequence located 3′ of the coding sequence for the polypeptide of interest, the construct comprises a splice acceptor site located 5′ of the coding sequence for the polypeptide of interest, and the nucleic acid construct does not comprise a promoter that drives expression of the polypeptide of interest, and optionally the nucleic acid construct does not comprise a homology arm.

### Multidomain Therapeutic Protein Nucleic Acid Constructs

The multidomain therapeutic protein nucleic acid constructs disclosed herein can be unidirectional constructs or bidirectional constructs. When specific construct sequences are disclosed herein, they are meant to encompass the sequence disclosed or the reverse complement of the sequence. For example, if a construct disclosed herein consists of the hypothetical sequence 5′-CTGGACCGA-3′, it is also meant to encompass the reverse complement of that sequence (5′-TCGGTCCAG-3′). Likewise, when construct elements are disclosed herein in a specific 5′ to 3′ order, they are also meant to encompass the reverse complement of the order of those elements. One reason for this is that, in many embodiments disclosed herein, the constructs are part of a single-stranded recombinant AAV vector. Single-stranded AAV genomes are packaged as either sense (plus-stranded) or anti-sense (minus-stranded genomes), and single-stranded AAV genomes of + and - polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

In the nucleic acid constructs, the multidomain therapeutic protein coding sequence, the CD63-binding delivery domain coding sequence, and/or the GAA coding sequence can be codon-optimized for expression in a host cell. For example, the multidomain therapeutic protein coding sequence, the CD63-binding delivery domain coding sequence, and/or the GAA coding sequence can be codon optimized or may use one or more alternative codons for one or more amino acids of the protein (i.e., same amino acid sequence). An alternative codon as used herein refers to variations in codon usage for a given amino acid, and may or may not be a preferred or optimized codon (codon optimized) for a given expression system. Preferred codon usage, or codons that are well-tolerated in a given system of expression, are known.

In the nucleic acid constructs, the multidomain therapeutic protein coding sequence, the TfR-binding delivery domain coding sequence, and/or the GAA coding sequence can be codon-optimized for expression in a host cell. For example, the multidomain therapeutic protein coding sequence, the TfR-binding delivery domain coding sequence, and/or the GAA coding sequence can be codon optimized or may use one or more alternative codons for one or more amino acids of the protein (i.e., same amino acid sequence). An alternative codon as used herein refers to variations in codon usage for a given amino acid, and may or may not be a preferred or optimized codon (codon optimized) for a given expression system. Preferred codon usage, or codons that are well-tolerated in a given system of expression, are known.

The nucleic acid constructs disclosed herein can be modified to include any suitable structural feature as needed for any particular use and/or that confers one or more desired functions. For example, the nucleic acid constructs disclosed herein need not comprise a homology arm and/or can be, for example, homology-independent donor constructs.

In some cases, the nucleic acid construct does not comprise a promoter that drives the expression of the multidomain therapeutic protein. For example, the expression of the multidomain therapeutic protein can be driven by a promoter of the host cell (e.g., the endogenous ALB promoter when the transgene is integrated into a host cell’s ALB locus). In other cases, the nucleic acid construct can comprise one or more promoters operably linked to the multidomain therapeutic protein coding sequence. That is, although not required for expression, the constructs disclosed herein may also include transcriptional or translational regulatory sequences such as promoters, enhancers, insulators, internal ribosome entry sites, additional sequences encoding peptides, and/or polyadenylation signals. Some nucleic acid constructs can comprise a promoter that drives expression of the multidomain therapeutic protein. For example, the promoter may be a liver-specific promoter. Examples of liver-specific promoters include TTR promoters, such as human or mouse TTR promoters. In one example, the construct may comprise a TTR promoter, such as a mouse TTR promoter or a human TTR promoter (e.g., the coding sequence for the multidomain therapeutic protein is operably linked to the TTR promoter). In one example, the construct may comprise a SERPINA1 enhancer, such as a mouse SERPINA1 enhancer or a human SERPINA1 enhancer (e.g., the coding sequence for the multidomain therapeutic protein is operably linked to the SERPINA1 enhancer). In one example, the construct may comprise a TTR promoter and a SERPINA1 enhancer, such as a human SERPINA1 enhancer and a mouse TTR promoter (e.g., the coding sequence for the multidomain therapeutic protein is operably linked to the SERPINA1 enhancer and the TTR promoter).

The nucleic acid constructs can, in some cases, comprise one or more polyadenylation tail sequences or polyadenylation signal sequences. Some nucleic acid constructs can comprise a polyadenylation signal sequence located 3′ of the multidomain therapeutic protein coding sequence. In a specific example, the polyadenylation signal is a simian virus 40 (SV40) late polyadenylation signal (or a variant thereof). In another specific example, the polyadenylation signal is a bovine growth hormone (BGH) polyadenylation signal (or a variant thereof). In another specific example, the polyadenylation signal is a CpG-depleted BGH polyadenylation signal. For example, the polyadenylation signal can be an SV40 polyadenylation signal or a CpG-depleted BGH polyadenylation signal. For example, the polyadenylation signal can comprise, consist essentially of, or consist of SEQ ID NO: 712, 169, or 161. In a specific example, the polyadenylation signal can comprise, consist essentially of, or consist of SEQ ID NO: 712. In a specific example, the polyadenylation signal can comprise, consist essentially of, or consist of SEQ ID NO: 169. In another specific example, the polyadenylation signal can comprise, consist essentially of, or consist of SEQ ID NO: 161. In another specific example, the polyadenylation signal can comprise, consist essentially of, or consist of SEQ ID NO: 162.

Methods of designing a suitable polyadenylation tail sequence are known. For example, some nucleic acid constructs comprise a polyadenylation tail sequence and/or a polyadenylation signal sequence downstream of an open reading frame (i.e., a polyadenylation tail sequence and/or a polyadenylation signal sequence 3′ of a coding sequence). The polyadenylation tail sequence can be encoded, for example, as a “poly-A” stretch downstream of the multidomain therapeutic protein coding sequence (or other protein coding sequence) in the first and/or second segment. A poly-A tail can comprise, for example, at least 20, 30, 40, 50, 60, 70, 80, 90, or 100 adenines, and optionally up to 300 adenines. In a specific example, the poly-A tail comprises 95, 96, 97, 98, 99, or 100 adenine nucleotides. Methods of designing a suitable polyadenylation tail sequence and/or polyadenylation signal sequence are well known. For example, the polyadenylation signal sequence AAUAAA is commonly used in mammalian systems, although variants such as UAUAAA or AU/GUAAA have been identified. See, e.g., Proudfoot (2011) Genes & Dev. 25(17):1770-82, herein incorporated by reference in its entirety for all purposes.

The nucleic acid constructs can, in some cases, comprise one or more splice acceptor sites. Some nucleic acid constructs comprise a splice acceptor site located 5′ of the multidomain therapeutic protein coding sequence. In a specific example, the splice acceptor is a mouse Alb exon 2 splice acceptor. In a specific example, the splice acceptor can comprise, consist essentially of, or consist of SEQ ID NO: 163.

The splice acceptor site can, for example, comprise NAG or consist of NAG. In a specific example, the splice acceptor is an ALB splice acceptor (e.g., an ALB splice acceptor used in the splicing together of exons 1 and 2 of ALB (i.e., ALB exon 2 splice acceptor)). For example, such a splice acceptor can be derived from the human ALB gene. In another example, the splice acceptor can be derived from the mouse Alb gene (e.g., an ALB splice acceptor used in the splicing together of exons 1 and 2 of mouse Alb (i.e., mouse Alb exon 2 splice acceptor)). In another example, the splice acceptor is a GAA splice acceptor. For example, such a splice acceptor can be derived from the human GAA gene. Alternatively, such a splice acceptor can be derived from the mouse GAA gene. Additional suitable splice acceptor sites useful in eukaryotes, including artificial splice acceptors, are known. See, e.g., Shapiro et al. (1987) Nucleic Acids Res. 15:7155-7174 and Burset et al. (2001) Nucleic Acids Res. 29:255-259, each of which is herein incorporated by reference in its entirety for all purposes.

The nucleic acid constructs can be circular or linear. For example, a nucleic acid construct can be linear. The nucleic acid constructs disclosed herein can be DNA or RNA, single-stranded, double-stranded, or partially single-stranded and partially double-stranded. For example, the constructs can be single- or double-stranded DNA. In some embodiments, the nucleic acid can be modified (e.g., using nucleoside analogs), as described herein. In a specific example, the nucleic acid construct is single-stranded (e.g., single-stranded DNA).

The nucleic acid constructs disclosed herein can be modified on either or both ends to include one or more suitable structural features as needed and/or to confer one or more functional benefit. For example, structural modifications can vary depending on the method(s) used to deliver the constructs disclosed herein to a host cell (e.g., use of viral vector delivery or packaging into lipid nanoparticles for delivery). Such modifications include, for example, terminal structures such as inverted terminal repeats (ITR), hairpin, loops, and other structures such as toroids. For example, the nucleic acid constructs disclosed herein can comprise one, two, or three ITRs or can comprise no more than two ITRs. Various methods of structural modifications are known.

Similarly, one or both ends of the nucleic acid construct can be protected (e.g., from exonucleolytic degradation) by known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in its entirety for all purposes. Additional methods for protecting the constructs from degradation include, but are not limited to, addition of terminal amino group(s) and the use of modified internucleotide linkages such as, for example, phosphorothioates, phosphoramidates, and O-methyl ribose or deoxyribose residues.

As disclosed in more detail herein, the nucleic acid constructs disclosed herein can be introduced into a cell as part of a vector having additional sequences such as, for example, replication origins, promoters, and genes encoding antibiotic resistance. The nucleic acid constructs can be introduced as a naked nucleic acid, can be introduced as a nucleic acid complexed with an agent such as a liposome, polymer, or poloxamer, or can be delivered by viral vectors (e.g., adenovirus, AAV, herpesvirus, retrovirus, lentivirus).

The multidomain therapeutic protein coding sequence, the CD63-binding delivery domain coding sequence, and/or the GAA coding sequence in the nucleic acid constructs disclosed herein may include one or more modifications such as codon optimization (e.g., to human codons), depletion of CpG dinucleotides, mutation of cryptic splice sites, addition of one or more glycosylation sites, or any combination thereof. CpG dinucleotides in a construct can limit the therapeutic utility of the construct. First, unmethylated CpG dinucleotides can interact with host toll-like receptor-9 (TLR-9) to stimulate innate, proinflammatory immune responses. Second, once the CpG dinucleotides become methylated, they can result in the suppression of transgene expression coordinated by methyl-CpG binding proteins. Cryptic splice sites are sequences in a pre-messenger RNA that are not normally used as splice sites, but that can be activated, for example, by mutations that either inactivate canonical splice sites or create splice sites where one did not exist before. Accurate splice site selection is critical for successful gene expression, and removal of cryptic splice sites can favor use of the normal or intended splice site.

In one example, a multidomain therapeutic protein coding sequence, a CD63-binding delivery domain coding sequence, and/or a GAA coding sequence in a nucleic acid construct disclosed herein has one or more cryptic splice sites mutated or removed. In another example, a multidomain therapeutic protein coding sequence, a CD63-binding delivery domain coding sequence, and/or a GAA coding sequence in a nucleic acid construct disclosed herein has all identified cryptic splice sites mutated or removed. In another example, a multidomain therapeutic protein coding sequence, a CD63-binding delivery domain coding sequence, and/or a GAA coding sequence in a nucleic acid construct disclosed herein has one or more CpG dinucleotides removed (i.e., is CpG depleted). In another example, a multidomain therapeutic protein coding sequence, a CD63-binding delivery domain coding sequence, and/or a GAA coding sequence in a nucleic acid construct disclosed herein has all CpG dinucleotides removed. In another example, a multidomain therapeutic protein coding sequence, a CD63-binding delivery domain coding sequence, and/or a GAA coding sequence in a nucleic acid construct disclosed herein is codon optimized (e.g., codon optimized for expression in a human or mammal). In a specific example, a multidomain therapeutic protein coding sequence, a CD63-binding delivery domain coding sequence, and/or a GAA coding sequence in a nucleic acid construct disclosed herein has one or more CpG dinucleotides removed (i.e., is CpG depleted) and has one or more cryptic splice sites mutated or removed. In another specific example, a multidomain therapeutic protein coding sequence, a CD63-binding delivery domain coding sequence, and/or a GAA coding sequence in a nucleic acid construct disclosed herein has all CpG dinucleotides removed and has one or more or all identified cryptic splice sites mutated or removed. In another specific example, a multidomain therapeutic protein coding sequence, a CD63-binding delivery domain coding sequence, and/or a GAA coding sequence in a nucleic acid construct disclosed herein has one or more CpG dinucleotides removed (i.e., is CpG depleted) and is codon optimized (e.g., codon optimized for expression in a human or mammal). In another specific example, a multidomain therapeutic protein coding sequence, a CD63-binding delivery domain coding sequence, and/or a GAA coding sequence in a nucleic acid construct disclosed herein has all CpG dinucleotides removed (i.e., is fully CpG depleted) and is codon optimized (e.g., codon optimized for expression in a human or mammal).

The multidomain therapeutic protein coding sequence, the TfR-binding delivery domain coding sequence, and/or the GAA coding sequence in the nucleic acid constructs disclosed herein may include one or more modifications such as codon optimization (e.g., to human codons), depletion of CpG dinucleotides, mutation of cryptic splice sites, addition of one or more glycosylation sites, or any combination thereof. CpG dinucleotides in a construct can limit the therapeutic utility of the construct. First, unmethylated CpG dinucleotides can interact with host toll-like receptor-9 (TLR-9) to stimulate innate, proinflammatory immune responses. Second, once the CpG dinucleotides become methylated, they can result in the suppression of transgene expression coordinated by methyl-CpG binding proteins. Cryptic splice sites are sequences in a pre-messenger RNA that are not normally used as splice sites, but that can be activated, for example, by mutations that either inactivate canonical splice sites or create splice sites where one did not exist before. Accurate splice site selection is critical for successful gene expression, and removal of cryptic splice sites can favor use of the normal or intended splice site.

In one example, a multidomain therapeutic protein coding sequence, a TfR-binding delivery domain coding sequence, and/or a GAA coding sequence in a nucleic acid construct disclosed herein has one or more cryptic splice sites mutated or removed. In another example, a multidomain therapeutic protein coding sequence, a TfR-binding delivery domain coding sequence, and/or a GAA coding sequence in a nucleic acid construct disclosed herein has all identified cryptic splice sites mutated or removed. In another example, a multidomain therapeutic protein coding sequence, a TfR-binding delivery domain coding sequence, and/or a GAA coding sequence in a nucleic acid construct disclosed herein has one or more CpG dinucleotides removed (i.e., is CpG depleted). In another example, a multidomain therapeutic protein coding sequence, a TfR-binding delivery domain coding sequence, and/or a GAA coding sequence in a nucleic acid construct disclosed herein has all CpG dinucleotides removed. In another example, a multidomain therapeutic protein coding sequence, a TfR-binding delivery domain coding sequence, and/or a GAA coding sequence in a nucleic acid construct disclosed herein is codon optimized (e.g., codon optimized for expression in a human or mammal). In a specific example, a multidomain therapeutic protein coding sequence, a TfR-binding delivery domain coding sequence, and/or a GAA coding sequence in a nucleic acid construct disclosed herein has one or more CpG dinucleotides removed (i.e., is CpG depleted) and has one or more cryptic splice sites mutated or removed. In another specific example, a multidomain therapeutic protein coding sequence, a TfR-binding delivery domain coding sequence, and/or a GAA coding sequence in a nucleic acid construct disclosed herein has all CpG dinucleotides removed and has one or more or all identified cryptic splice sites mutated or removed. In another specific example, a multidomain therapeutic protein coding sequence, a TfR-binding delivery domain coding sequence, and/or a GAA coding sequence in a nucleic acid construct disclosed herein has one or more CpG dinucleotides removed (i.e., is CpG depleted) and is codon optimized (e.g., codon optimized for expression in a human or mammal). In another specific example, a multidomain therapeutic protein coding sequence, a TfR-binding delivery domain coding sequence, and/or a GAA coding sequence in a nucleic acid construct disclosed herein has all CpG dinucleotides removed (i.e., is fully CpG depleted) and is codon optimized (e.g., codon optimized for expression in a human or mammal).

In an exemplary nucleic acid construct, the construct comprises a polyadenylation signal sequence located 3′ of the multidomain therapeutic protein coding sequence, the construct comprises a splice acceptor site located 5′ of the multidomain therapeutic protein coding sequence, and the nucleic acid construct does not comprise a promoter that drives expression of the multidomain therapeutic protein, and optionally the nucleic acid construct does not comprise a homology arm.

In a specific example of a multidomain therapeutic protein nucleic acid construct, the encoded multidomain therapeutic protein can comprise SEQ ID NO: 193 or can be at least 90%, at least 91%, at least 92%, at least 93%, at least 94%, at least 95%, at least 96%, at least 97%, at least 98%, at least 99%, or at least 99.5% identical to SEQ ID NO: 193. In another specific example, the multidomain therapeutic protein can consist essentially of SEQ ID NO: 193. In another specific example, the multidomain therapeutic protein can consist of SEQ ID NO: 193.

Various multidomain therapeutic protein coding sequences are provided. In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 194-202. In another example, the multidomain therapeutic protein coding sequence is (or comprises a sequence) at least 95%, at least 96%, at least 97%, at least 98%, at least 99%, at least 99.5%, or 100% identical to any one of SEQ ID NOS: 194-202. In another example, the multidomain therapeutic protein coding sequence is (or comprises a sequence) at least 99%, at least 99.5%, or 100% identical to any one of SEQ ID NOS: 194-202. In another example, the multidomain therapeutic protein coding sequence comprises the sequence set forth in any one of SEQ ID NOS: 194-202. In another example, the multidomain therapeutic protein coding sequence consists essentially of the sequence set forth in any one of SEQ ID NOS: 194-202. In another example, the multidomain therapeutic protein coding sequence consists of the sequence set forth in any one of SEQ ID NOS: 194-202. In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 196. In another example, the multidomain therapeutic protein coding sequence is (or comprises a sequence) at least 95%, at least 96%, at least 97%, at least 98%, at least 99%, at least 99.5%, or 100% identical to SEQ ID NO: 196. In another example, the multidomain therapeutic protein coding sequence is (or comprises a sequence) at least 99%, at least 99.5%, or 100% identical to SEQ ID NO: 196. In another example, the multidomain therapeutic protein coding sequence comprises the sequence set forth in SEQ ID NO: 196. In another example, the multidomain therapeutic protein coding sequence consists essentially of the sequence set forth in SEQ ID NO: 196. In another example, the multidomain therapeutic protein coding sequence consists of the sequence set forth in SEQ ID NO: 196. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 193 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 193 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 193 (and, e.g., retaining the activity of native GAA). Optionally, the multidomain therapeutic protein coding sequence in the above examples encodes a multidomain therapeutic protein comprising the sequence set forth in SEQ ID NO: 193. Optionally, the multidomain therapeutic protein coding sequence in the above examples encodes a multidomain therapeutic protein consisting essentially of the sequence set forth in SEQ ID NO: 193. Optionally, the multidomain therapeutic protein coding sequence in the above examples encodes a multidomain therapeutic protein consisting of the sequence set forth in SEQ ID NO: 193.

Various codon optimized multidomain therapeutic protein coding sequences are provided. The multidomain therapeutic protein coding sequence can be, for example, CpG-depleted (e.g., fully CpG depleted) and/or codon optimized (e.g., CpG depleted (e.g., fully CpG-depleted) and codon optimized). In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 195-202. In another example, the multidomain therapeutic protein coding sequence is (or comprises a sequence) at least 95%, at least 96%, at least 97%, at least 98%, at least 99%, at least 99.5%, or 100% identical to any one of SEQ ID NOS: 195-202. In another example, the multidomain therapeutic protein coding sequence is (or comprises a sequence) at least 99%, at least 99.5%, or 100% identical to any one of SEQ ID NOS: 195-202. In another example, the multidomain therapeutic protein coding sequence comprises the sequence set forth in any one of SEQ ID NOS: 195-202. In another example, the multidomain therapeutic protein coding sequence consists essentially of the sequence set forth in any one of SEQ ID NOS: 195-202. In another example, the multidomain therapeutic protein coding sequence consists of the sequence set forth in any one of SEQ ID NOS: 195-202. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 193 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 193 (and, e.g., retaining the activity of native GAA). Optionally, the GAA coding sequence in the above examples encodes a multidomain therapeutic protein (or a multidomain therapeutic protein comprising a sequence) at least 99%, at least 99.5%, or 100% identical to SEQ ID NO: 193 (and, e.g., retaining the activity of native GAA). Optionally, the multidomain therapeutic protein coding sequence in the above examples encodes a multidomain therapeutic protein comprising the sequence set forth in SEQ ID NO: 193. Optionally, the multidomain therapeutic protein coding sequence in the above examples encodes a multidomain therapeutic protein consisting essentially of the sequence set forth in SEQ ID NO: 193. Optionally, the multidomain therapeutic protein coding sequence in the above examples encodes a multidomain therapeutic protein consisting of the sequence set forth in SEQ ID NO: 193.

In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 196. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 196 and encodes a multidomain therapeutic protein (or a multidomain therapeutic protein comprising a sequence) at least 99%, at least 99.5%, or 100% identical to SEQ ID NO: 193. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 196 and encodes a multidomain therapeutic protein comprising the sequence set forth in SEQ ID NO: 193. In another example, the multidomain therapeutic protein coding sequence is (or comprises a sequence) at least 95%, at least 96%, at least 97%, at least 98%, at least 99%, at least 99.5%, or 100% identical to SEQ ID NO: 196. In another example, the multidomain therapeutic protein coding sequence is (or comprises a sequence) at least 95%, at least 96%, at least 97%, at least 98%, at least 99%, at least 99.5%, or 100% identical to SEQ ID NO: 196 and encodes a multidomain therapeutic protein (or a multidomain therapeutic protein comprising a sequence) at least 99%, at least 99.5%, or 100% identical to SEQ ID NO: 193. In another example, the multidomain therapeutic protein coding sequence is (or comprises a sequence) at least 95%, at least 96%, at least 97%, at least 98%, at least 99%, at least 99.5%, or 100% identical to SEQ ID NO: 196 and encodes a multidomain therapeutic protein comprising the sequence set forth in SEQ ID NO: 193. In another example, the multidomain therapeutic protein coding sequence is (or comprises a sequence) at least 99%, at least 99.5%, or 100% identical to SEQ ID NO: 196. In another example, the multidomain therapeutic protein coding sequence is (or comprises a sequence) at least 99%, at least 99.5%, or 100% identical to SEQ ID NO: 196 and encodes a multidomain therapeutic protein (or a multidomain therapeutic protein comprising a sequence) at least 99%, at least 99.5%, or 100% identical to SEQ ID NO: 193. In another example, the multidomain therapeutic protein coding sequence is (or comprises a sequence) at least 99%, at least 99.5%, or 100% identical to SEQ ID NO: 196 and encodes a multidomain therapeutic protein comprising the sequence set forth in SEQ ID NO: 193. In another example, the multidomain therapeutic protein coding sequence comprises the sequence set forth in SEQ ID NO: 196. In another example, the multidomain therapeutic protein coding sequence consists essentially of the sequence set forth in SEQ ID NO: 196. In another example, the multidomain therapeutic protein coding sequence consists of the sequence set forth in SEQ ID NO: 196. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 193 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 193 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 193 (and, e.g., retaining the activity of native GAA). Optionally, the multidomain therapeutic protein coding sequence in the above examples encodes a multidomain therapeutic protein comprising the sequence set forth in SEQ ID NO: 193. Optionally, the multidomain therapeutic protein coding sequence in the above examples encodes a multidomain therapeutic protein consisting essentially of the sequence set forth in SEQ ID NO: 193. Optionally, the multidomain therapeutic protein coding sequence in the above examples encodes a multidomain therapeutic protein consisting of the sequence set forth in SEQ ID NO: 193.

The nucleic acid construct can comprise, for example, (1) a 5′ ITR (e.g., such as the one set forth in SEQ ID NO: 160), (2) a splice acceptor site (e.g., a mouse Alb exon 2 splice acceptor, such as the one set forth in SEQ ID NO: 163), (3) the multidomain therapeutic protein coding sequence, (4) a polyadenylation signal (e.g., an SV40 polyadenylation signal, such as the one set forth in SEQ ID NO: 712), and (5) a 3′ ITR (e.g., such as the one set forth in SEQ ID NO: 160 or the reverse complement thereof). In one example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 736. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 736 and encodes a multidomain therapeutic protein (or a multidomain therapeutic protein comprising a sequence) at least 99%, at least 99.5%, or 100% identical to SEQ ID NO: 193. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 736 and encodes a multidomain therapeutic protein comprising the sequence set forth in SEQ ID NO: 193. In another example, the nucleic acid construct comprises a sequence at least 95%, at least 96%, at least 97%, at least 98%, at least 99%, at least 99.5%, or 100% identical to SEQ ID NO: 736. In another example, the nucleic acid construct comprises a sequence at least 95%, at least 96%, at least 97%, at least 98%, at least 99%, at least 99.5%, or 100% identical to SEQ ID NO: 736 and encodes a multidomain therapeutic protein (or a multidomain therapeutic protein comprising a sequence) at least 99%, at least 99.5%, or 100% identical to SEQ ID NO: 193. In another example, the nucleic acid construct comprises a sequence at least 95%, at least 96%, at least 97%, at least 98%, at least 99%, at least 99.5%, or 100% identical to SEQ ID NO: 736 and encodes a multidomain therapeutic protein comprising the sequence set forth in SEQ ID NO: 193. In another example, the nucleic acid construct comprises a sequence at least 99%, at least 99.5%, or 100% identical to SEQ ID NO: 736. In another example, the nucleic acid construct comprises a sequence at least 99%, at least 99.5%, or 100% identical to SEQ ID NO: 736 and encodes a multidomain therapeutic protein (or a multidomain therapeutic protein comprising a sequence) at least 99%, at least 99.5%, or 100% identical to SEQ ID NO: 193. In another example, the nucleic acid construct comprises a sequence at least 99%, at least 99.5%, or 100% identical to SEQ ID NO: 736 and encodes a multidomain therapeutic protein comprising the sequence set forth in SEQ ID NO: 193. In another example, the nucleic acid construct comprises the sequence set forth in SEQ ID NO: 736. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the nucleic acid construct encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 193 (and, e.g., retaining the activity of native GAA). Optionally, the nucleic acid construct encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 193 (and, e.g., retaining the activity of native GAA). Optionally, the nucleic acid construct in the above examples encodes a multidomain therapeutic protein (or a multidomain therapeutic protein comprising a sequence) at least 99%, at least 99.5%, or 100% identical to SEQ ID NO: 193 (and, e.g., retaining the activity of native GAA). Optionally, the nucleic acid construct in the above examples encodes a multidomain therapeutic protein comprising the sequence set forth in SEQ ID NO: 193. Optionally, the nucleic acid construct in the above examples encodes a multidomain therapeutic protein consisting essentially of the sequence set forth in SEQ ID NO: 193. Optionally, the nucleic acid construct in the above examples encodes a multidomain therapeutic protein consisting of the sequence set forth in SEQ ID NO: 193.

In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 194. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 194 and encodes a multidomain therapeutic protein (or a multidomain therapeutic protein comprising a sequence) at least 99%, at least 99.5%, or 100% identical to SEQ ID NO: 193. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 194 and encodes a multidomain therapeutic protein comprising the sequence set forth in SEQ ID NO: 193. In another example, the multidomain therapeutic protein coding sequence is (or comprises a sequence) at least 95%, at least 96%, at least 97%, at least 98%, at least 99%, at least 99.5%, or 100% identical to SEQ ID NO: 194. In another example, the multidomain therapeutic protein coding sequence is (or comprises a sequence) at least 95%, at least 96%, at least 97%, at least 98%, at least 99%, at least 99.5%, or 100% identical to SEQ ID NO: 194 and encodes a multidomain therapeutic protein (or a multidomain therapeutic protein comprising a sequence) at least 99%, at least 99.5%, or 100% identical to SEQ ID NO: 193. In another example, the multidomain therapeutic protein coding sequence is (or comprises a sequence) at least 95%, at least 96%, at least 97%, at least 98%, at least 99%, at least 99.5%, or 100% identical to SEQ ID NO: 194 and encodes a multidomain therapeutic protein comprising the sequence set forth in SEQ ID NO: 193. In another example, the multidomain therapeutic protein coding sequence is (or comprises a sequence) at least 99%, at least 99.5%, or 100% identical to SEQ ID NO: 194. In another example, the multidomain therapeutic protein coding sequence is (or comprises a sequence) at least 99%, at least 99.5%, or 100% identical to SEQ ID NO: 194 and encodes a multidomain therapeutic protein (or a multidomain therapeutic protein comprising a sequence) at least 99%, at least 99.5%, or 100% identical to SEQ ID NO: 193. In another example, the multidomain therapeutic protein coding sequence is (or comprises a sequence) at least 99%, at least 99.5%, or 100% identical to SEQ ID NO: 194 and encodes a multidomain therapeutic protein comprising the sequence set forth in SEQ ID NO: 193. In another example, the multidomain therapeutic protein coding sequence comprises the sequence set forth in SEQ ID NO: 194. In another example, the multidomain therapeutic protein coding sequence consists essentially of the sequence set forth in SEQ ID NO: 194. In another example, the multidomain therapeutic protein coding sequence consists of the sequence set forth in SEQ ID NO: 194. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 193 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 193 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 193 (and, e.g., retaining the activity of native GAA). Optionally, the multidomain therapeutic protein coding sequence in the above examples encodes a multidomain therapeutic protein comprising the sequence set forth in SEQ ID NO: 193. Optionally, the multidomain therapeutic protein coding sequence in the above examples encodes a multidomain therapeutic protein consisting essentially of the sequence set forth in SEQ ID NO: 193. Optionally, the multidomain therapeutic protein coding sequence in the above examples encodes a multidomain therapeutic protein consisting of the sequence set forth in SEQ ID NO: 193.

In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 201. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 201 and encodes a multidomain therapeutic protein (or a multidomain therapeutic protein comprising a sequence) at least 99%, at least 99.5%, or 100% identical to SEQ ID NO: 193. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 201 and encodes a multidomain therapeutic protein comprising the sequence set forth in SEQ ID NO: 193. In another example, the multidomain therapeutic protein coding sequence is (or comprises a sequence) at least 95%, at least 96%, at least 97%, at least 98%, at least 99%, at least 99.5%, or 100% identical to SEQ ID NO: 201. In another example, the multidomain therapeutic protein coding sequence is (or comprises a sequence) at least 95%, at least 96%, at least 97%, at least 98%, at least 99%, at least 99.5%, or 100% identical to SEQ ID NO: 201 and encodes a multidomain therapeutic protein (or a multidomain therapeutic protein comprising a sequence) at least 99%, at least 99.5%, or 100% identical to SEQ ID NO: 193. In another example, the multidomain therapeutic protein coding sequence is (or comprises a sequence) at least 95%, at least 96%, at least 97%, at least 98%, at least 99%, at least 99.5%, or 100% identical to SEQ ID NO: 201 and encodes a multidomain therapeutic protein comprising the sequence set forth in SEQ ID NO: 193. In another example, the multidomain therapeutic protein coding sequence is (or comprises a sequence) at least 99%, at least 99.5%, or 100% identical to SEQ ID NO: 201. In another example, the multidomain therapeutic protein coding sequence is (or comprises a sequence) at least 99%, at least 99.5%, or 100% identical to SEQ ID NO: 201 and encodes a multidomain therapeutic protein (or a multidomain therapeutic protein comprising a sequence) at least 99%, at least 99.5%, or 100% identical to SEQ ID NO: 193. In another example, the multidomain therapeutic protein coding sequence is (or comprises a sequence) at least 99%, at least 99.5%, or 100% identical to SEQ ID NO: 201 and encodes a multidomain therapeutic protein comprising the sequence set forth in SEQ ID NO: 193. In another example, the multidomain therapeutic protein coding sequence comprises the sequence set forth in SEQ ID NO: 201. In another example, the multidomain therapeutic protein coding sequence consists essentially of the sequence set forth in SEQ ID NO: 201. In another example, the multidomain therapeutic protein coding sequence consists of the sequence set forth in SEQ ID NO: 201. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 193 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 193 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 193 (and, e.g., retaining the activity of native GAA). Optionally, the multidomain therapeutic protein coding sequence in the above examples encodes a multidomain therapeutic protein comprising the sequence set forth in SEQ ID NO: 193. Optionally, the multidomain therapeutic protein coding sequence in the above examples encodes a multidomain therapeutic protein consisting essentially of the sequence set forth in SEQ ID NO: 193. Optionally, the multidomain therapeutic protein coding sequence in the above examples encodes a multidomain therapeutic protein consisting of the sequence set forth in SEQ ID NO: 193.

In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 200. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 200 and encodes a multidomain therapeutic protein (or a multidomain therapeutic protein comprising a sequence) at least 99%, at least 99.5%, or 100% identical to SEQ ID NO: 193. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 200 and encodes a multidomain therapeutic protein comprising the sequence set forth in SEQ ID NO: 193. In another example, the multidomain therapeutic protein coding sequence is (or comprises a sequence) at least 95%, at least 96%, at least 97%, at least 98%, at least 99%, at least 99.5%, or 100% identical to SEQ ID NO: 200. In another example, the multidomain therapeutic protein coding sequence is (or comprises a sequence) at least 95%, at least 96%, at least 97%, at least 98%, at least 99%, at least 99.5%, or 100% identical to SEQ ID NO: 200 and encodes a multidomain therapeutic protein (or a multidomain therapeutic protein comprising a sequence) at least 99%, at least 99.5%, or 100% identical to SEQ ID NO: 193. In another example, the multidomain therapeutic protein coding sequence is (or comprises a sequence) at least 95%, at least 96%, at least 97%, at least 98%, at least 99%, at least 99.5%, or 100% identical to SEQ ID NO: 200 and encodes a multidomain therapeutic protein comprising the sequence set forth in SEQ ID NO: 193. In another example, the multidomain therapeutic protein coding sequence is (or comprises a sequence) at least 99%, at least 99.5%, or 100% identical to SEQ ID NO: 200. In another example, the multidomain therapeutic protein coding sequence is (or comprises a sequence) at least 99%, at least 99.5%, or 100% identical to SEQ ID NO: 200 and encodes a multidomain therapeutic protein (or a multidomain therapeutic protein comprising a sequence) at least 99%, at least 99.5%, or 100% identical to SEQ ID NO: 193. In another example, the multidomain therapeutic protein coding sequence is (or comprises a sequence) at least 99%, at least 99.5%, or 100% identical to SEQ ID NO: 200 and encodes a multidomain therapeutic protein comprising the sequence set forth in SEQ ID NO: 193. In another example, the multidomain therapeutic protein coding sequence comprises the sequence set forth in SEQ ID NO: 200. In another example, the multidomain therapeutic protein coding sequence consists essentially of the sequence set forth in SEQ ID NO: 200. In another example, the multidomain therapeutic protein coding sequence consists of the sequence set forth in SEQ ID NO: 200. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 193 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 193 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 193 (and, e.g., retaining the activity of native GAA). Optionally, the multidomain therapeutic protein coding sequence in the above examples encodes a multidomain therapeutic protein comprising the sequence set forth in SEQ ID NO: 193. Optionally, the multidomain therapeutic protein coding sequence in the above examples encodes a multidomain therapeutic protein consisting essentially of the sequence set forth in SEQ ID NO: 193. Optionally, the multidomain therapeutic protein coding sequence in the above examples encodes a multidomain therapeutic protein consisting of the sequence set forth in SEQ ID NO: 193.

In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 198. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 198 and encodes a multidomain therapeutic protein (or a multidomain therapeutic protein comprising a sequence) at least 99%, at least 99.5%, or 100% identical to SEQ ID NO: 193. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 198 and encodes a multidomain therapeutic protein comprising the sequence set forth in SEQ ID NO: 193. In another example, the multidomain therapeutic protein coding sequence is (or comprises a sequence) at least 95%, at least 96%, at least 97%, at least 98%, at least 99%, at least 99.5%, or 100% identical to SEQ ID NO: 198. In another example, the multidomain therapeutic protein coding sequence is (or comprises a sequence) at least 95%, at least 96%, at least 97%, at least 98%, at least 99%, at least 99.5%, or 100% identical to SEQ ID NO: 198 and encodes a multidomain therapeutic protein (or a multidomain therapeutic protein comprising a sequence) at least 99%, at least 99.5%, or 100% identical to SEQ ID NO: 193. In another example, the multidomain therapeutic protein coding sequence is (or comprises a sequence) at least 95%, at least 96%, at least 97%, at least 98%, at least 99%, at least 99.5%, or 100% identical to SEQ ID NO: 198 and encodes a multidomain therapeutic protein comprising the sequence set forth in SEQ ID NO: 193. In another example, the multidomain therapeutic protein coding sequence is (or comprises a sequence) at least 99%, at least 99.5%, or 100% identical to SEQ ID NO: 198. In another example, the multidomain therapeutic protein coding sequence is (or comprises a sequence) at least 99%, at least 99.5%, or 100% identical to SEQ ID NO: 198 and encodes a multidomain therapeutic protein (or a multidomain therapeutic protein comprising a sequence) at least 99%, at least 99.5%, or 100% identical to SEQ ID NO: 193. In another example, the multidomain therapeutic protein coding sequence is (or comprises a sequence) at least 99%, at least 99.5%, or 100% identical to SEQ ID NO: 198 and encodes a multidomain therapeutic protein comprising the sequence set forth in SEQ ID NO: 193. In another example, the multidomain therapeutic protein coding sequence comprises the sequence set forth in SEQ ID NO: 198. In another example, the multidomain therapeutic protein coding sequence consists essentially of the sequence set forth in SEQ ID NO: 198. In another example, the multidomain therapeutic protein coding sequence consists of the sequence set forth in SEQ ID NO: 198. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 193 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 193 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 193 (and, e.g., retaining the activity of native GAA). Optionally, the multidomain therapeutic protein coding sequence in the above examples encodes a multidomain therapeutic protein comprising the sequence set forth in SEQ ID NO: 193. Optionally, the multidomain therapeutic protein coding sequence in the above examples encodes a multidomain therapeutic protein consisting essentially of the sequence set forth in SEQ ID NO: 193. Optionally, the multidomain therapeutic protein coding sequence in the above examples encodes a multidomain therapeutic protein consisting of the sequence set forth in SEQ ID NO: 193.

In some cases, the anti-hTfR:GAA scFv fusion proteins in the format V_{L}-(Gly₄Ser)₃-V_{H}:GAA (Gly₄Ser = SEQ ID NO: 600). In a specific example of a multidomain therapeutic protein nucleic acid construct, the encoded multidomain therapeutic protein can comprise any one of SEQ ID NOS: 570-573 and 675-702 or can be at least 90%, at least 91%, at least 92%, at least 93%, at least 94%, at least 95%, at least 96%, at least 97%, at least 98%, at least 99%, or at least 99.5% identical to any one of SEQ ID NOS: 570-573 and 675-702. In another specific example, the multidomain therapeutic protein can consist essentially of any one of SEQ ID NOS: 570-573 and 675-702. In another specific example, the multidomain therapeutic protein can consist of any one of SEQ ID NOS: 570-573 and 675-702.

In a specific example of a multidomain therapeutic protein nucleic acid construct, the encoded multidomain therapeutic protein can comprise SEQ ID NO: 570 or can be at least 90%, at least 91%, at least 92%, at least 93%, at least 94%, at least 95%, at least 96%, at least 97%, at least 98%, at least 99%, or at least 99.5% identical to SEQ ID NO: 570. In another specific example, the multidomain therapeutic protein can consist essentially of SEQ ID NO: 570. In another specific example, the multidomain therapeutic protein can consist of SEQ ID NO: 570.

In a specific example of a multidomain therapeutic protein nucleic acid construct, the encoded multidomain therapeutic protein can comprise SEQ ID NO: 571 or can be at least 90%, at least 91%, at least 92%, at least 93%, at least 94%, at least 95%, at least 96%, at least 97%, at least 98%, at least 99%, or at least 99.5% identical to SEQ ID NO: 571. In another specific example, the multidomain therapeutic protein can consist essentially of SEQ ID NO: 571. In another specific example, the multidomain therapeutic protein can consist of SEQ ID NO: 571.

In a specific example of a multidomain therapeutic protein nucleic acid construct, the encoded multidomain therapeutic protein can comprise SEQ ID NO: 572 or can be at least 90%, at least 91%, at least 92%, at least 93%, at least 94%, at least 95%, at least 96%, at least 97%, at least 98%, at least 99%, or at least 99.5% identical to SEQ ID NO: 572. In another specific example, the multidomain therapeutic protein can consist essentially of SEQ ID NO: 572. In another specific example, the multidomain therapeutic protein can consist of SEQ ID NO: 572.

In a specific example of a multidomain therapeutic protein nucleic acid construct, the encoded multidomain therapeutic protein can comprise SEQ ID NO: 573or can be at least 90%, at least 91%, at least 92%, at least 93%, at least 94%, at least 95%, at least 96%, at least 97%, at least 98%, at least 99%, or at least 99.5% identical to SEQ ID NO: 573. In another specific example, the multidomain therapeutic protein can consist essentially of SEQ ID NO: 573. In another specific example, the multidomain therapeutic protein can consist of SEQ ID NO: 573.

In some cases, the anti-hTfR:GAA scFv fusion proteins in the format V_{H}-(Gly₄Ser)₃-V_{L}:GAA (Gly₄Ser = SEQ ID NO: 600). In a specific example of a multidomain therapeutic protein nucleic acid construct, the encoded multidomain therapeutic protein can comprise any one of SEQ ID NOS: 703-706 (optionally lacking the N-terminal MHRPRRRGTRPPPLALLAALLLAARGADA sequence) or can be at least 90%, at least 91%, at least 92%, at least 93%, at least 94%, at least 95%, at least 96%, at least 97%, at least 98%, at least 99%, or at least 99.5% identical to any one of SEQ ID NOS: 703-706 (optionally lacking the N-terminal MHRPRRRGTRPPPLALLAALLLAARGADA sequence). In another specific example, the multidomain therapeutic protein can consist essentially of any one of SEQ ID NOS: 703-706 (optionally lacking the N-terminal MHRPRRRGTRPPPLALLAALLLAARGADA sequence). In another specific example, the multidomain therapeutic protein can consist of any one of SEQ ID NOS: 703-706 (optionally lacking the N-terminal MHRPRRRGTRPPPLALLAALLLAARGADA sequence).

Various multidomain therapeutic protein coding sequences are provided. In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 574-586. In another example, the multidomain therapeutic protein coding sequence is (or comprises a sequence) at least 95%, at least 96%, at least 97%, at least 98%, at least 99%, at least 99.5%, or 100% identical to any one of SEQ ID NOS: 574-586. In another example, the multidomain therapeutic protein coding sequence is (or comprises a sequence) at least 99%, at least 99.5%, or 100% identical to any one of SEQ ID NOS: 574-586. In another example, the multidomain therapeutic protein coding sequence comprises the sequence set forth in any one of SEQ ID NOS: 574-586. In another example, the multidomain therapeutic protein coding sequence consists essentially of the sequence set forth in any one of SEQ ID NOS: 574-586. In another example, the multidomain therapeutic protein coding sequence consists of the sequence set forth in any one of SEQ ID NOS: 574-586. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 570-573 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to any one of SEQ ID NOS: 570-573 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to any one of SEQ ID NOS: 570-573 (and, e.g., retaining the activity of native GAA). Optionally, the multidomain therapeutic protein coding sequence in the above examples encodes a multidomain therapeutic protein comprising the sequence set forth in any one of SEQ ID NOS: 570-573. Optionally, the multidomain therapeutic protein coding sequence in the above examples encodes a multidomain therapeutic protein consisting essentially of the sequence set forth in any one of SEQ ID NOS: 570-573. Optionally, the multidomain therapeutic protein coding sequence in the above examples encodes a multidomain therapeutic protein consisting of the sequence set forth in any one of SEQ ID NOS: 570-573.

Various codon optimized multidomain therapeutic protein coding sequences are provided. The multidomain therapeutic protein coding sequence can be, for example, CpG-depleted (e.g., fully CpG depleted) and/or codon optimized (e.g., CpG depleted (e.g., fully CpG-depleted) and codon optimized). In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 578-586. In another example, the multidomain therapeutic protein coding sequence is (or comprises a sequence) at least 95%, at least 96%, at least 97%, at least 98%, at least 99%, at least 99.5%, or 100% identical to any one of SEQ ID NOS: 578-586. In another example, the multidomain therapeutic protein coding sequence is (or comprises a sequence) at least 99%, at least 99.5%, or 100% identical to any one of SEQ ID NOS: 578-586. In another example, the multidomain therapeutic protein coding sequence comprises the sequence set forth in any one of SEQ ID NOS: 578-586. In another example, the multidomain therapeutic protein coding sequence consists essentially of the sequence set forth in any one of SEQ ID NOS: 578-586. In another example, the multidomain therapeutic protein coding sequence consists of the sequence set forth in any one of SEQ ID NOS: 578-586. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 570-573 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to any one of SEQ ID NOS: 570-573 (and, e.g., retaining the activity of native GAA). Optionally, the GAA coding sequence in the above examples encodes a multidomain therapeutic protein (or a multidomain therapeutic protein comprising a sequence) at least 99%, at least 99.5%, or 100% identical to any one of SEQ ID NOS: 570-573 (and, e.g., retaining the activity of native GAA). Optionally, the multidomain therapeutic protein coding sequence in the above examples encodes a multidomain therapeutic protein comprising the sequence set forth in any one of SEQ ID NOS: 570-573. Optionally, the multidomain therapeutic protein coding sequence in the above examples encodes a multidomain therapeutic protein consisting essentially of the sequence set forth in any one of SEQ ID NOS: 570-573. Optionally, the multidomain therapeutic protein coding sequence in the above examples encodes a multidomain therapeutic protein consisting of the sequence set forth in any one of SEQ ID NOS: 570-573.

Various multidomain therapeutic protein coding sequences are provided. In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 577 and 584-586. In another example, the multidomain therapeutic protein coding sequence is (or comprises a sequence) at least 95%, at least 96%, at least 97%, at least 98%, at least 99%, at least 99.5%, or 100% identical to any one of SEQ ID NOS: 577 and 584-586. In another example, the multidomain therapeutic protein coding sequence is (or comprises a sequence) at least 99%, at least 99.5%, or 100% identical to any one of SEQ ID NOS: 577 and 584-586. In another example, the multidomain therapeutic protein coding sequence comprises the sequence set forth in any one of SEQ ID NOS: 577 and 584-586. In another example, the multidomain therapeutic protein coding sequence consists essentially of the sequence set forth in any one of SEQ ID NOS: 577 and 584-586. In another example, the multidomain therapeutic protein coding sequence consists of the sequence set forth in any one of SEQ ID NOS: 577 and 584-586. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 573 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 573 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 573 (and, e.g., retaining the activity of native GAA). Optionally, the multidomain therapeutic protein coding sequence in the above examples encodes a multidomain therapeutic protein comprising the sequence set forth in SEQ ID NO: 573. Optionally, the multidomain therapeutic protein coding sequence in the above examples encodes a multidomain therapeutic protein consisting essentially of the sequence set forth in SEQ ID NO: 573. Optionally, the multidomain therapeutic protein coding sequence in the above examples encodes a multidomain therapeutic protein consisting of the sequence set forth in SEQ ID NO: 573.

Various codon optimized multidomain therapeutic protein coding sequences are provided. The multidomain therapeutic protein coding sequence can be, for example, CpG-depleted (e.g., fully CpG depleted) and/or codon optimized (e.g., CpG depleted (e.g., fully CpG-depleted) and codon optimized). In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 584-586. In another example, the multidomain therapeutic protein coding sequence is (or comprises a sequence) at least 95%, at least 96%, at least 97%, at least 98%, at least 99%, at least 99.5%, or 100% identical to any one of SEQ ID NOS: 584-586. In another example, the multidomain therapeutic protein coding sequence is (or comprises a sequence) at least 99%, at least 99.5%, or 100% identical to any one of SEQ ID NOS: 584-586. In another example, the multidomain therapeutic protein coding sequence comprises the sequence set forth in any one of SEQ ID NOS: 584-586. In another example, the multidomain therapeutic protein coding sequence consists essentially of the sequence set forth in any one of SEQ ID NOS: 584-586. In another example, the multidomain therapeutic protein coding sequence consists of the sequence set forth in any one of SEQ ID NOS: 584-586. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 573 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 573 (and, e.g., retaining the activity of native GAA). Optionally, the GAA coding sequence in the above examples encodes a multidomain therapeutic protein (or a multidomain therapeutic protein comprising a sequence) at least 99%, at least 99.5%, or 100% identical to SEQ ID NO: 573 (and, e.g., retaining the activity of native GAA). Optionally, the multidomain therapeutic protein coding sequence in the above examples encodes a multidomain therapeutic protein comprising the sequence set forth in SEQ ID NO: 573. Optionally, the multidomain therapeutic protein coding sequence in the above examples encodes a multidomain therapeutic protein consisting essentially of the sequence set forth in SEQ ID NO: 573. Optionally, the multidomain therapeutic protein coding sequence in the above examples encodes a multidomain therapeutic protein consisting of the sequence set forth in SEQ ID NO: 573.

In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 584. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 584 and encodes a multidomain therapeutic protein (or a multidomain therapeutic protein comprising a sequence) at least 99%, at least 99.5%, or 100% identical to SEQ ID NO: 573. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 584 and encodes a multidomain therapeutic protein comprising the sequence set forth in SEQ ID NO: 573. In another example, the multidomain therapeutic protein coding sequence is (or comprises a sequence) at least 95%, at least 96%, at least 97%, at least 98%, at least 99%, at least 99.5%, or 100% identical to SEQ ID NO: 584. In another example, the multidomain therapeutic protein coding sequence is (or comprises a sequence) at least 95%, at least 96%, at least 97%, at least 98%, at least 99%, at least 99.5%, or 100% identical to SEQ ID NO: 584 and encodes a multidomain therapeutic protein (or a multidomain therapeutic protein comprising a sequence) at least 99%, at least 99.5%, or 100% identical to SEQ ID NO: 573. In another example, the multidomain therapeutic protein coding sequence is (or comprises a sequence) at least 95%, at least 96%, at least 97%, at least 98%, at least 99%, at least 99.5%, or 100% identical to SEQ ID NO: 584 and encodes a multidomain therapeutic protein comprising the sequence set forth in SEQ ID NO: 573. In another example, the multidomain therapeutic protein coding sequence is (or comprises a sequence) at least 99%, at least 99.5%, or 100% identical to SEQ ID NO: 584. In another example, the multidomain therapeutic protein coding sequence is (or comprises a sequence) at least 99%, at least 99.5%, or 100% identical to SEQ ID NO: 584 and encodes a multidomain therapeutic protein (or a multidomain therapeutic protein comprising a sequence) at least 99%, at least 99.5%, or 100% identical to SEQ ID NO: 573. In another example, the multidomain therapeutic protein coding sequence is (or comprises a sequence) at least 99%, at least 99.5%, or 100% identical to SEQ ID NO: 584 and encodes a multidomain therapeutic protein comprising the sequence set forth in SEQ ID NO: 573. In another example, the multidomain therapeutic protein coding sequence comprises the sequence set forth in SEQ ID NO: 584. In another example, the multidomain therapeutic protein coding sequence consists essentially of the sequence set forth in SEQ ID NO: 584. In another example, the multidomain therapeutic protein coding sequence consists of the sequence set forth in SEQ ID NO: 584. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 573 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 573 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 573 (and, e.g., retaining the activity of native GAA). Optionally, the multidomain therapeutic protein coding sequence in the above examples encodes a multidomain therapeutic protein comprising the sequence set forth in SEQ ID NO: 573. Optionally, the multidomain therapeutic protein coding sequence in the above examples encodes a multidomain therapeutic protein consisting essentially of the sequence set forth in SEQ ID NO: 573. Optionally, the multidomain therapeutic protein coding sequence in the above examples encodes a multidomain therapeutic protein consisting of the sequence set forth in SEQ ID NO: 573.

The nucleic acid construct can comprise, for example, (1) a 5′ ITR (e.g., such as the one set forth in SEQ ID NO: 160), (2) a splice acceptor site (e.g., a mouse Alb exon 2 splice acceptor, such as the one set forth in SEQ ID NO: 163), (3) the multidomain therapeutic protein coding sequence, (4) a polyadenylation signal (e.g., an SV40 polyadenylation signal, such as the one set forth in SEQ ID NO: 712), and (5) a 3′ ITR (e.g., such as the one set forth in SEQ ID NO: 160 or the reverse complement thereof). In one example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 733. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 733 and encodes a multidomain therapeutic protein (or a multidomain therapeutic protein comprising a sequence) at least 99%, at least 99.5%, or 100% identical to SEQ ID NO: 573. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 733 and encodes a multidomain therapeutic protein comprising the sequence set forth in SEQ ID NO: 573. In another example, the nucleic acid construct comprises a sequence at least 95%, at least 96%, at least 97%, at least 98%, at least 99%, at least 99.5%, or 100% identical to SEQ ID NO: 733. In another example, the nucleic acid construct comprises a sequence at least 95%, at least 96%, at least 97%, at least 98%, at least 99%, at least 99.5%, or 100% identical to SEQ ID NO: 733 and encodes a multidomain therapeutic protein (or a multidomain therapeutic protein comprising a sequence) at least 99%, at least 99.5%, or 100% identical to SEQ ID NO: 573. In another example, the nucleic acid construct comprises a sequence at least 95%, at least 96%, at least 97%, at least 98%, at least 99%, at least 99.5%, or 100% identical to SEQ ID NO: 733 and encodes a multidomain therapeutic protein comprising the sequence set forth in SEQ ID NO: 573. In another example, the nucleic acid construct comprises a sequence at least 99%, at least 99.5%, or 100% identical to SEQ ID NO: 733. In another example, the nucleic acid construct comprises a sequence at least 99%, at least 99.5%, or 100% identical to SEQ ID NO: 733 and encodes a multidomain therapeutic protein (or a multidomain therapeutic protein comprising a sequence) at least 99%, at least 99.5%, or 100% identical to SEQ ID NO: 573. In another example, the nucleic acid construct comprises a sequence at least 99%, at least 99.5%, or 100% identical to SEQ ID NO: 733 and encodes a multidomain therapeutic protein comprising the sequence set forth in SEQ ID NO: 573. In another example, the nucleic acid construct comprises the sequence set forth in SEQ ID NO: 733. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the nucleic acid construct encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 573 (and, e.g., retaining the activity of native GAA). Optionally, the nucleic acid construct encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 573 (and, e.g., retaining the activity of native GAA). Optionally, the nucleic acid construct in the above examples encodes a multidomain therapeutic protein (or a multidomain therapeutic protein comprising a sequence) at least 99%, at least 99.5%, or 100% identical to SEQ ID NO: 573 (and, e.g., retaining the activity of native GAA). Optionally, the nucleic acid construct in the above examples encodes a multidomain therapeutic protein comprising the sequence set forth in SEQ ID NO: 573. Optionally, the nucleic acid construct in the above examples encodes a multidomain therapeutic protein consisting essentially of the sequence set forth in SEQ ID NO: 573. Optionally, the nucleic acid construct in the above examples encodes a multidomain therapeutic protein consisting of the sequence set forth in SEQ ID NO: 573.

In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 585. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 585 and encodes a multidomain therapeutic protein (or a multidomain therapeutic protein comprising a sequence) at least 99%, at least 99.5%, or 100% identical to SEQ ID NO: 573. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 585 and encodes a multidomain therapeutic protein comprising the sequence set forth in SEQ ID NO: 573. In another example, the multidomain therapeutic protein coding sequence is (or comprises a sequence) at least 95%, at least 96%, at least 97%, at least 98%, at least 99%, at least 99.5%, or 100% identical to SEQ ID NO: 585. In another example, the multidomain therapeutic protein coding sequence is (or comprises a sequence) at least 95%, at least 96%, at least 97%, at least 98%, at least 99%, at least 99.5%, or 100% identical to SEQ ID NO: 585 and encodes a multidomain therapeutic protein (or a multidomain therapeutic protein comprising a sequence) at least 99%, at least 99.5%, or 100% identical to SEQ ID NO: 573. In another example, the multidomain therapeutic protein coding sequence is (or comprises a sequence) at least 95%, at least 96%, at least 97%, at least 98%, at least 99%, at least 99.5%, or 100% identical to SEQ ID NO: 585 and encodes a multidomain therapeutic protein comprising the sequence set forth in SEQ ID NO: 573. In another example, the multidomain therapeutic protein coding sequence is (or comprises a sequence) at least 99%, at least 99.5%, or 100% identical to SEQ ID NO: 585. In another example, the multidomain therapeutic protein coding sequence is (or comprises a sequence) at least 99%, at least 99.5%, or 100% identical to SEQ ID NO: 585 and encodes a multidomain therapeutic protein (or a multidomain therapeutic protein comprising a sequence) at least 99%, at least 99.5%, or 100% identical to SEQ ID NO: 573. In another example, the multidomain therapeutic protein coding sequence is (or comprises a sequence) at least 99%, at least 99.5%, or 100% identical to SEQ ID NO: 585 and encodes a multidomain therapeutic protein comprising the sequence set forth in SEQ ID NO: 573. In another example, the multidomain therapeutic protein coding sequence comprises the sequence set forth in SEQ ID NO: 585. In another example, the multidomain therapeutic protein coding sequence consists essentially of the sequence set forth in SEQ ID NO: 585. In another example, the multidomain therapeutic protein coding sequence consists of the sequence set forth in SEQ ID NO: 585. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 573 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 573 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 573 (and, e.g., retaining the activity of native GAA). Optionally, the multidomain therapeutic protein coding sequence in the above examples encodes a multidomain therapeutic protein comprising the sequence set forth in SEQ ID NO: 573. Optionally, the multidomain therapeutic protein coding sequence in the above examples encodes a multidomain therapeutic protein consisting essentially of the sequence set forth in SEQ ID NO: 573. Optionally, the multidomain therapeutic protein coding sequence in the above examples encodes a multidomain therapeutic protein consisting of the sequence set forth in SEQ ID NO: 573.

The nucleic acid construct can comprise, for example, (1) a 5′ ITR (e.g., such as the one set forth in SEQ ID NO: 160), (2) a splice acceptor site (e.g., a mouse Alb exon 2 splice acceptor, such as the one set forth in SEQ ID NO: 163), (3) the multidomain therapeutic protein coding sequence, (4) a polyadenylation signal (e.g., an SV40 polyadenylation signal, such as the one set forth in SEQ ID NO: 712), and (5) a 3′ ITR (e.g., such as the one set forth in SEQ ID NO: 160 or the reverse complement thereof). In one example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 734. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 734 and encodes a multidomain therapeutic protein (or a multidomain therapeutic protein comprising a sequence) at least 99%, at least 99.5%, or 100% identical to SEQ ID NO: 573. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 734 and encodes a multidomain therapeutic protein comprising the sequence set forth in SEQ ID NO: 573. In another example, the nucleic acid construct comprises a sequence at least 95%, at least 96%, at least 97%, at least 98%, at least 99%, at least 99.5%, or 100% identical to SEQ ID NO: 734. In another example, the nucleic acid construct comprises a sequence at least 95%, at least 96%, at least 97%, at least 98%, at least 99%, at least 99.5%, or 100% identical to SEQ ID NO: 734 and encodes a multidomain therapeutic protein (or a multidomain therapeutic protein comprising a sequence) at least 99%, at least 99.5%, or 100% identical to SEQ ID NO: 573. In another example, the nucleic acid construct comprises a sequence at least 95%, at least 96%, at least 97%, at least 98%, at least 99%, at least 99.5%, or 100% identical to SEQ ID NO: 734 and encodes a multidomain therapeutic protein comprising the sequence set forth in SEQ ID NO: 573. In another example, the nucleic acid construct comprises a sequence at least 99%, at least 99.5%, or 100% identical to SEQ ID NO: 734. In another example, the nucleic acid construct comprises a sequence at least 99%, at least 99.5%, or 100% identical to SEQ ID NO: 734 and encodes a multidomain therapeutic protein (or a multidomain therapeutic protein comprising a sequence) at least 99%, at least 99.5%, or 100% identical to SEQ ID NO: 573. In another example, the nucleic acid construct comprises a sequence at least 99%, at least 99.5%, or 100% identical to SEQ ID NO: 734 and encodes a multidomain therapeutic protein comprising the sequence set forth in SEQ ID NO: 573. In another example, the nucleic acid construct comprises the sequence set forth in SEQ ID NO: 734. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the nucleic acid construct encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 573 (and, e.g., retaining the activity of native GAA). Optionally, the nucleic acid construct encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 573 (and, e.g., retaining the activity of native GAA). Optionally, the nucleic acid construct in the above examples encodes a multidomain therapeutic protein (or a multidomain therapeutic protein comprising a sequence) at least 99%, at least 99.5%, or 100% identical to SEQ ID NO: 573 (and, e.g., retaining the activity of native GAA). Optionally, the nucleic acid construct in the above examples encodes a multidomain therapeutic protein comprising the sequence set forth in SEQ ID NO: 573. Optionally, the nucleic acid construct in the above examples encodes a multidomain therapeutic protein consisting essentially of the sequence set forth in SEQ ID NO: 573. Optionally, the nucleic acid construct in the above examples encodes a multidomain therapeutic protein consisting of the sequence set forth in SEQ ID NO: 573.

In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 586. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 586 and encodes a multidomain therapeutic protein (or a multidomain therapeutic protein comprising a sequence) at least 99%, at least 99.5%, or 100% identical to SEQ ID NO: 573. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 586 and encodes a multidomain therapeutic protein comprising the sequence set forth in SEQ ID NO: 573. In another example, the multidomain therapeutic protein coding sequence is (or comprises a sequence) at least 95%, at least 96%, at least 97%, at least 98%, at least 99%, at least 99.5%, or 100% identical to SEQ ID NO: 586. In another example, the multidomain therapeutic protein coding sequence is (or comprises a sequence) at least 95%, at least 96%, at least 97%, at least 98%, at least 99%, at least 99.5%, or 100% identical to SEQ ID NO: 586 and encodes a multidomain therapeutic protein (or a multidomain therapeutic protein comprising a sequence) at least 99%, at least 99.5%, or 100% identical to SEQ ID NO: 573. In another example, the multidomain therapeutic protein coding sequence is (or comprises a sequence) at least 95%, at least 96%, at least 97%, at least 98%, at least 99%, at least 99.5%, or 100% identical to SEQ ID NO: 586 and encodes a multidomain therapeutic protein comprising the sequence set forth in SEQ ID NO: 573. In another example, the multidomain therapeutic protein coding sequence is (or comprises a sequence) at least 99%, at least 99.5%, or 100% identical to SEQ ID NO: 586. In another example, the multidomain therapeutic protein coding sequence is (or comprises a sequence) at least 99%, at least 99.5%, or 100% identical to SEQ ID NO: 586 and encodes a multidomain therapeutic protein (or a multidomain therapeutic protein comprising a sequence) at least 99%, at least 99.5%, or 100% identical to SEQ ID NO: 573. In another example, the multidomain therapeutic protein coding sequence is (or comprises a sequence) at least 99%, at least 99.5%, or 100% identical to SEQ ID NO: 586 and encodes a multidomain therapeutic protein comprising the sequence set forth in SEQ ID NO: 573. In another example, the multidomain therapeutic protein coding sequence comprises the sequence set forth in SEQ ID NO: 586. In another example, the multidomain therapeutic protein coding sequence consists essentially of the sequence set forth in SEQ ID NO: 586. In another example, the multidomain therapeutic protein coding sequence consists of the sequence set forth in SEQ ID NO: 586. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 573 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 573 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 573 (and, e.g., retaining the activity of native GAA). Optionally, the multidomain therapeutic protein coding sequence in the above examples encodes a multidomain therapeutic protein comprising the sequence set forth in SEQ ID NO: 573. Optionally, the multidomain therapeutic protein coding sequence in the above examples encodes a multidomain therapeutic protein consisting essentially of the sequence set forth in SEQ ID NO: 573. Optionally, the multidomain therapeutic protein coding sequence in the above examples encodes a multidomain therapeutic protein consisting of the sequence set forth in SEQ ID NO: 573.

The nucleic acid construct can comprise, for example, (1) a 5′ ITR (e.g., such as the one set forth in SEQ ID NO: 160), (2) a splice acceptor site (e.g., a mouse Alb exon 2 splice acceptor, such as the one set forth in SEQ ID NO: 163), (3) the multidomain therapeutic protein coding sequence, (4) a polyadenylation signal (e.g., an SV40 polyadenylation signal, such as the one set forth in SEQ ID NO: 712), and (5) a 3′ ITR (e.g., such as the one set forth in SEQ ID NO: 160 or the reverse complement thereof). In one example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 735. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 735 and encodes a multidomain therapeutic protein (or a multidomain therapeutic protein comprising a sequence) at least 99%, at least 99.5%, or 100% identical to SEQ ID NO: 573. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 735 and encodes a multidomain therapeutic protein comprising the sequence set forth in SEQ ID NO: 573. In another example, the nucleic acid construct comprises a sequence at least 95%, at least 96%, at least 97%, at least 98%, at least 99%, at least 99.5%, or 100% identical to SEQ ID NO: 735. In another example, nucleic acid construct comprises a sequence at least 95%, at least 96%, at least 97%, at least 98%, at least 99%, at least 99.5%, or 100% identical to SEQ ID NO: 735 and encodes a multidomain therapeutic protein (or a multidomain therapeutic protein comprising a sequence) at least 99%, at least 99.5%, or 100% identical to SEQ ID NO: 573. In another example, the nucleic acid construct comprises a sequence at least 95%, at least 96%, at least 97%, at least 98%, at least 99%, at least 99.5%, or 100% identical to SEQ ID NO: 735 and encodes a multidomain therapeutic protein comprising the sequence set forth in SEQ ID NO: 573. In another example, the nucleic acid construct comprises a sequence at least 99%, at least 99.5%, or 100% identical to SEQ ID NO: 735. In another example, the nucleic acid construct comprises a sequence at least 99%, at least 99.5%, or 100% identical to SEQ ID NO: 735 and encodes a multidomain therapeutic protein (or a multidomain therapeutic protein comprising a sequence) at least 99%, at least 99.5%, or 100% identical to SEQ ID NO: 573. In another example, the nucleic acid construct comprises a sequence at least 99%, at least 99.5%, or 100% identical to SEQ ID NO: 735 and encodes a multidomain therapeutic protein comprising the sequence set forth in SEQ ID NO: 573. In another example, the nucleic acid construct comprises the sequence set forth in SEQ ID NO: 735. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the nucleic acid construct encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 573 (and, e.g., retaining the activity of native GAA). Optionally, the nucleic acid construct encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 573 (and, e.g., retaining the activity of native GAA). Optionally, the nucleic acid construct in the above examples encodes a multidomain therapeutic protein (or a multidomain therapeutic protein comprising a sequence) at least 99%, at least 99.5%, or 100% identical to SEQ ID NO: 573 (and, e.g., retaining the activity of native GAA). Optionally, the nucleic acid construct in the above examples encodes a multidomain therapeutic protein comprising the sequence set forth in SEQ ID NO: 573. Optionally, the nucleic acid construct in the above examples encodes a multidomain therapeutic protein consisting essentially of the sequence set forth in SEQ ID NO: 573. Optionally, the nucleic acid construct in the above examples encodes a multidomain therapeutic protein consisting of the sequence set forth in SEQ ID NO: 573.

Various multidomain therapeutic protein coding sequences are provided. In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 576 and 581-583. In another example, the multidomain therapeutic protein coding sequence is (or comprises a sequence) at least 95%, at least 96%, at least 97%, at least 98%, at least 99%, at least 99.5%, or 100% identical to any one of SEQ ID NOS: 576 and 581-583. In another example, the multidomain therapeutic protein coding sequence is (or comprises a sequence) at least 99%, at least 99.5%, or 100% identical to any one of SEQ ID NOS: 576 and 581-583. In another example, the multidomain therapeutic protein coding sequence comprises the sequence set forth in any one of SEQ ID NOS: 576 and 581-583. In another example, the multidomain therapeutic protein coding sequence consists essentially of the sequence set forth in any one of SEQ ID NOS: 576 and 581-583. In another example, the multidomain therapeutic protein coding sequence consists of the sequence set forth in any one of SEQ ID NOS: 576 and 581-583. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 572 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 572 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 572 (and, e.g., retaining the activity of native GAA). Optionally, the multidomain therapeutic protein coding sequence in the above examples encodes a multidomain therapeutic protein comprising the sequence set forth in SEQ ID NO: 572. Optionally, the multidomain therapeutic protein coding sequence in the above examples encodes a multidomain therapeutic protein consisting essentially of the sequence set forth in SEQ ID NO: 572. Optionally, the multidomain therapeutic protein coding sequence in the above examples encodes a multidomain therapeutic protein consisting of the sequence set forth in SEQ ID NO: 572.

Various codon optimized multidomain therapeutic protein coding sequences are provided. The multidomain therapeutic protein coding sequence can be, for example, CpG-depleted (e.g., fully CpG depleted) and/or codon optimized (e.g., CpG depleted (e.g., fully CpG-depleted) and codon optimized). In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 581-583. In another example, the multidomain therapeutic protein coding sequence is (or comprises a sequence) at least 95%, at least 96%, at least 97%, at least 98%, at least 99%, at least 99.5%, or 100% identical to any one of SEQ ID NOS: 581-583. In another example, the multidomain therapeutic protein coding sequence is (or comprises a sequence) at least 99%, at least 99.5%, or 100% identical to any one of SEQ ID NOS: 581-583. In another example, the multidomain therapeutic protein coding sequence comprises the sequence set forth in any one of SEQ ID NOS: 581-583. In another example, the multidomain therapeutic protein coding sequence consists essentially of the sequence set forth in any one of SEQ ID NOS: 581-583. In another example, the multidomain therapeutic protein coding sequence consists of the sequence set forth in any one of SEQ ID NOS: 581-583. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 572 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 572 (and, e.g., retaining the activity of native GAA). Optionally, the GAA coding sequence in the above examples encodes a multidomain therapeutic protein (or a multidomain therapeutic protein comprising a sequence) at least 99%, at least 99.5%, or 100% identical to SEQ ID NO: 572 (and, e.g., retaining the activity of native GAA). Optionally, the multidomain therapeutic protein coding sequence in the above examples encodes a multidomain therapeutic protein comprising the sequence set forth in SEQ ID NO: 572. Optionally, the multidomain therapeutic protein coding sequence in the above examples encodes a multidomain therapeutic protein consisting essentially of the sequence set forth in SEQ ID NO: 572. Optionally, the multidomain therapeutic protein coding sequence in the above examples encodes a multidomain therapeutic protein consisting of the sequence set forth in SEQ ID NO: 572.

In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 581. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 581 and encodes a multidomain therapeutic protein (or a multidomain therapeutic protein comprising a sequence) at least 99%, at least 99.5%, or 100% identical to SEQ ID NO: 572. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 581 and encodes a multidomain therapeutic protein comprising the sequence set forth in SEQ ID NO: 572. In another example, the multidomain therapeutic protein coding sequence is (or comprises a sequence) at least 95%, at least 96%, at least 97%, at least 98%, at least 99%, at least 99.5%, or 100% identical to SEQ ID NO: 581. In another example, the multidomain therapeutic protein coding sequence is (or comprises a sequence) at least 95%, at least 96%, at least 97%, at least 98%, at least 99%, at least 99.5%, or 100% identical to SEQ ID NO: 581 and encodes a multidomain therapeutic protein (or a multidomain therapeutic protein comprising a sequence) at least 99%, at least 99.5%, or 100% identical to SEQ ID NO: 572. In another example, the multidomain therapeutic protein coding sequence is (or comprises a sequence) at least 95%, at least 96%, at least 97%, at least 98%, at least 99%, at least 99.5%, or 100% identical to SEQ ID NO: 581 and encodes a multidomain therapeutic protein comprising the sequence set forth in SEQ ID NO: 572. In another example, the multidomain therapeutic protein coding sequence is (or comprises a sequence) at least 99%, at least 99.5%, or 100% identical to SEQ ID NO: 581. In another example, the multidomain therapeutic protein coding sequence is (or comprises a sequence) at least 99%, at least 99.5%, or 100% identical to SEQ ID NO: 581 and encodes a multidomain therapeutic protein (or a multidomain therapeutic protein comprising a sequence) at least 99%, at least 99.5%, or 100% identical to SEQ ID NO: 572. In another example, the multidomain therapeutic protein coding sequence is (or comprises a sequence) at least 99%, at least 99.5%, or 100% identical to SEQ ID NO: 581 and encodes a multidomain therapeutic protein comprising the sequence set forth in SEQ ID NO: 572. In another example, the multidomain therapeutic protein coding sequence comprises the sequence set forth in SEQ ID NO: 581. In another example, the multidomain therapeutic protein coding sequence consists essentially of the sequence set forth in SEQ ID NO: 581. In another example, the multidomain therapeutic protein coding sequence consists of the sequence set forth in SEQ ID NO: 581. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 572 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 572 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 572 (and, e.g., retaining the activity of native GAA). Optionally, the multidomain therapeutic protein coding sequence in the above examples encodes a multidomain therapeutic protein comprising the sequence set forth in SEQ ID NO: 572. Optionally, the multidomain therapeutic protein coding sequence in the above examples encodes a multidomain therapeutic protein consisting essentially of the sequence set forth in SEQ ID NO: 572. Optionally, the multidomain therapeutic protein coding sequence in the above examples encodes a multidomain therapeutic protein consisting of the sequence set forth in SEQ ID NO: 572.

The nucleic acid construct can comprise, for example, (1) a 5′ ITR (e.g., such as the one set forth in SEQ ID NO: 160), (2) a splice acceptor site (e.g., a mouse Alb exon 2 splice acceptor, such as the one set forth in SEQ ID NO: 163), (3) the multidomain therapeutic protein coding sequence, (4) a polyadenylation signal (e.g., an SV40 polyadenylation signal, such as the one set forth in SEQ ID NO: 712), and (5) a 3′ ITR (e.g., such as the one set forth in SEQ ID NO: 160 or the reverse complement thereof). In one example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 729. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 729 and encodes a multidomain therapeutic protein (or a multidomain therapeutic protein comprising a sequence) at least 99%, at least 99.5%, or 100% identical to SEQ ID NO: 572. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 729 and encodes a multidomain therapeutic protein comprising the sequence set forth in SEQ ID NO: 572. In another example, the nucleic acid construct comprises a sequence at least 95%, at least 96%, at least 97%, at least 98%, at least 99%, at least 99.5%, or 100% identical to SEQ ID NO: 729. In another example, the nucleic acid construct comprises a sequence at least 95%, at least 96%, at least 97%, at least 98%, at least 99%, at least 99.5%, or 100% identical to SEQ ID NO: 729 and encodes a multidomain therapeutic protein (or a multidomain therapeutic protein comprising a sequence) at least 99%, at least 99.5%, or 100% identical to SEQ ID NO: 572. In another example, the nucleic acid construct comprises a sequence at least 95%, at least 96%, at least 97%, at least 98%, at least 99%, at least 99.5%, or 100% identical to SEQ ID NO: 729 and encodes a multidomain therapeutic protein comprising the sequence set forth in SEQ ID NO: 572. In another example, the nucleic acid construct comprises a sequence at least 99%, at least 99.5%, or 100% identical to SEQ ID NO: 729. In another example, the nucleic acid construct comprises a sequence at least 99%, at least 99.5%, or 100% identical to SEQ ID NO: 729 and encodes a multidomain therapeutic protein (or a multidomain therapeutic protein comprising a sequence) at least 99%, at least 99.5%, or 100% identical to SEQ ID NO: 572. In another example, the nucleic acid construct comprises a sequence at least 99%, at least 99.5%, or 100% identical to SEQ ID NO: 729 and encodes a multidomain therapeutic protein comprising the sequence set forth in SEQ ID NO: 572. In another example, the nucleic acid construct comprises the sequence set forth in SEQ ID NO: 729. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the nucleic acid construct encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 572 (and, e.g., retaining the activity of native GAA). Optionally, the nucleic acid construct encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 572 (and, e.g., retaining the activity of native GAA). Optionally, the nucleic acid construct in the above examples encodes a multidomain therapeutic protein (or a multidomain therapeutic protein comprising a sequence) at least 99%, at least 99.5%, or 100% identical to SEQ ID NO: 572 (and, e.g., retaining the activity of native GAA). Optionally, the nucleic acid construct in the above examples encodes a multidomain therapeutic protein comprising the sequence set forth in SEQ ID NO: 572. Optionally, the nucleic acid construct in the above examples encodes a multidomain therapeutic protein consisting essentially of the sequence set forth in SEQ ID NO: 572. Optionally, the nucleic acid construct in the above examples encodes a multidomain therapeutic protein consisting of the sequence set forth in SEQ ID NO: 572.

In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 582. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 582 and encodes a multidomain therapeutic protein (or a multidomain therapeutic protein comprising a sequence) at least 99%, at least 99.5%, or 100% identical to SEQ ID NO: 572. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 582 and encodes a multidomain therapeutic protein comprising the sequence set forth in SEQ ID NO: 572. In another example, the multidomain therapeutic protein coding sequence is (or comprises a sequence) at least 95%, at least 96%, at least 97%, at least 98%, at least 99%, at least 99.5%, or 100% identical to SEQ ID NO: 582. In another example, the multidomain therapeutic protein coding sequence is (or comprises a sequence) at least 95%, at least 96%, at least 97%, at least 98%, at least 99%, at least 99.5%, or 100% identical to SEQ ID NO: 582 and encodes a multidomain therapeutic protein (or a multidomain therapeutic protein comprising a sequence) at least 99%, at least 99.5%, or 100% identical to SEQ ID NO: 572. In another example, the multidomain therapeutic protein coding sequence is (or comprises a sequence) at least 95%, at least 96%, at least 97%, at least 98%, at least 99%, at least 99.5%, or 100% identical to SEQ ID NO: 582 and encodes a multidomain therapeutic protein comprising the sequence set forth in SEQ ID NO: 572. In another example, the multidomain therapeutic protein coding sequence is (or comprises a sequence) at least 99%, at least 99.5%, or 100% identical to SEQ ID NO: 582. In another example, the multidomain therapeutic protein coding sequence is (or comprises a sequence) at least 99%, at least 99.5%, or 100% identical to SEQ ID NO: 582 and encodes a multidomain therapeutic protein (or a multidomain therapeutic protein comprising a sequence) at least 99%, at least 99.5%, or 100% identical to SEQ ID NO: 572. In another example, the multidomain therapeutic protein coding sequence is (or comprises a sequence) at least 99%, at least 99.5%, or 100% identical to SEQ ID NO: 582 and encodes a multidomain therapeutic protein comprising the sequence set forth in SEQ ID NO: 572. In another example, the multidomain therapeutic protein coding sequence comprises the sequence set forth in SEQ ID NO: 582. In another example, the multidomain therapeutic protein coding sequence consists essentially of the sequence set forth in SEQ ID NO: 582. In another example, the multidomain therapeutic protein coding sequence consists of the sequence set forth in SEQ ID NO: 582. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 572 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 572 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 572 (and, e.g., retaining the activity of native GAA). Optionally, the multidomain therapeutic protein coding sequence in the above examples encodes a multidomain therapeutic protein comprising the sequence set forth in SEQ ID NO: 572. Optionally, the multidomain therapeutic protein coding sequence in the above examples encodes a multidomain therapeutic protein consisting essentially of the sequence set forth in SEQ ID NO: 572. Optionally, the multidomain therapeutic protein coding sequence in the above examples encodes a multidomain therapeutic protein consisting of the sequence set forth in SEQ ID NO: 572.

The nucleic acid construct can comprise, for example, (1) a 5′ ITR (e.g., such as the one set forth in SEQ ID NO: 160), (2) a splice acceptor site (e.g., a mouse Alb exon 2 splice acceptor, such as the one set forth in SEQ ID NO: 163), (3) the multidomain therapeutic protein coding sequence, (4) a polyadenylation signal (e.g., an SV40 polyadenylation signal, such as the one set forth in SEQ ID NO: 712), and (5) a 3′ ITR (e.g., such as the one set forth in SEQ ID NO: 160 or the reverse complement thereof). In one example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 730. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 730 and encodes a multidomain therapeutic protein (or a multidomain therapeutic protein comprising a sequence) at least 99%, at least 99.5%, or 100% identical to SEQ ID NO: 572. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 730 and encodes a multidomain therapeutic protein comprising the sequence set forth in SEQ ID NO: 572. In another example, the nucleic acid construct comprises a sequence at least 95%, at least 96%, at least 97%, at least 98%, at least 99%, at least 99.5%, or 100% identical to SEQ ID NO: 730. In another example, the nucleic acid construct comprises a sequence at least 95%, at least 96%, at least 97%, at least 98%, at least 99%, at least 99.5%, or 100% identical to SEQ ID NO: 730 and encodes a multidomain therapeutic protein (or a multidomain therapeutic protein comprising a sequence) at least 99%, at least 99.5%, or 100% identical to SEQ ID NO: 572. In another example, the nucleic acid construct comprises a sequence at least 95%, at least 96%, at least 97%, at least 98%, at least 99%, at least 99.5%, or 100% identical to SEQ ID NO: 730 and encodes a multidomain therapeutic protein comprising the sequence set forth in SEQ ID NO: 572. In another example, the nucleic acid construct comprises a sequence at least 99%, at least 99.5%, or 100% identical to SEQ ID NO: 730. In another example, the nucleic acid construct comprises a sequence at least 99%, at least 99.5%, or 100% identical to SEQ ID NO: 730 and encodes a multidomain therapeutic protein (or a multidomain therapeutic protein comprising a sequence) at least 99%, at least 99.5%, or 100% identical to SEQ ID NO: 572. In another example, the nucleic acid construct comprises a sequence at least 99%, at least 99.5%, or 100% identical to SEQ ID NO: 730 and encodes a multidomain therapeutic protein comprising the sequence set forth in SEQ ID NO: 572. In another example, the nucleic acid construct comprises the sequence set forth in SEQ ID NO: 730. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the nucleic acid construct encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 572 (and, e.g., retaining the activity of native GAA). Optionally, the nucleic acid construct encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 572 (and, e.g., retaining the activity of native GAA). Optionally, the nucleic acid construct in the above examples encodes a multidomain therapeutic protein (or a multidomain therapeutic protein comprising a sequence) at least 99%, at least 99.5%, or 100% identical to SEQ ID NO: 572 (and, e.g., retaining the activity of native GAA). Optionally, the nucleic acid construct in the above examples encodes a multidomain therapeutic protein comprising the sequence set forth in SEQ ID NO: 572. Optionally, the nucleic acid construct in the above examples encodes a multidomain therapeutic protein consisting essentially of the sequence set forth in SEQ ID NO: 572. Optionally, the nucleic acid construct in the above examples encodes a multidomain therapeutic protein consisting of the sequence set forth in SEQ ID NO: 572.

In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 583. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 583 and encodes a multidomain therapeutic protein (or a multidomain therapeutic protein comprising a sequence) at least 99%, at least 99.5%, or 100% identical to SEQ ID NO: 572. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 583 and encodes a multidomain therapeutic protein comprising the sequence set forth in SEQ ID NO: 572. In another example, the multidomain therapeutic protein coding sequence is (or comprises a sequence) at least 95%, at least 96%, at least 97%, at least 98%, at least 99%, at least 99.5%, or 100% identical to SEQ ID NO: 583. In another example, the multidomain therapeutic protein coding sequence is (or comprises a sequence) at least 95%, at least 96%, at least 97%, at least 98%, at least 99%, at least 99.5%, or 100% identical to SEQ ID NO: 583 and encodes a multidomain therapeutic protein (or a multidomain therapeutic protein comprising a sequence) at least 99%, at least 99.5%, or 100% identical to SEQ ID NO: 572. In another example, the multidomain therapeutic protein coding sequence is (or comprises a sequence) at least 95%, at least 96%, at least 97%, at least 98%, at least 99%, at least 99.5%, or 100% identical to SEQ ID NO: 583 and encodes a multidomain therapeutic protein comprising the sequence set forth in SEQ ID NO: 572. In another example, the multidomain therapeutic protein coding sequence is (or comprises a sequence) at least 99%, at least 99.5%, or 100% identical to SEQ ID NO: 583. In another example, the multidomain therapeutic protein coding sequence is (or comprises a sequence) at least 99%, at least 99.5%, or 100% identical to SEQ ID NO: 583 and encodes a multidomain therapeutic protein (or a multidomain therapeutic protein comprising a sequence) at least 99%, at least 99.5%, or 100% identical to SEQ ID NO: 572. In another example, the multidomain therapeutic protein coding sequence is (or comprises a sequence) at least 99%, at least 99.5%, or 100% identical to SEQ ID NO: 583 and encodes a multidomain therapeutic protein comprising the sequence set forth in SEQ ID NO: 572. In another example, the multidomain therapeutic protein coding sequence comprises the sequence set forth in SEQ ID NO: 583. In another example, the multidomain therapeutic protein coding sequence consists essentially of the sequence set forth in SEQ ID NO: 583. In another example, the multidomain therapeutic protein coding sequence consists of the sequence set forth in SEQ ID NO: 583. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 572 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 572 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 572 (and, e.g., retaining the activity of native GAA). Optionally, the multidomain therapeutic protein coding sequence in the above examples encodes a multidomain therapeutic protein comprising the sequence set forth in SEQ ID NO: 572. Optionally, the multidomain therapeutic protein coding sequence in the above examples encodes a multidomain therapeutic protein consisting essentially of the sequence set forth in SEQ ID NO: 572. Optionally, the multidomain therapeutic protein coding sequence in the above examples encodes a multidomain therapeutic protein consisting of the sequence set forth in SEQ ID NO: 572.

The nucleic acid construct can comprise, for example, (1) a 5′ ITR (e.g., such as the one set forth in SEQ ID NO: 160), (2) a splice acceptor site (e.g., a mouse Alb exon 2 splice acceptor, such as the one set forth in SEQ ID NO: 163), (3) the multidomain therapeutic protein coding sequence, (4) a polyadenylation signal (e.g., an SV40 polyadenylation signal, such as the one set forth in SEQ ID NO: 712), and (5) a 3′ ITR (e.g., such as the one set forth in SEQ ID NO: 160 or the reverse complement thereof). In one example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 731. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 731 and encodes a multidomain therapeutic protein (or a multidomain therapeutic protein comprising a sequence) at least 99%, at least 99.5%, or 100% identical to SEQ ID NO: 572. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 731 and encodes a multidomain therapeutic protein comprising the sequence set forth in SEQ ID NO: 572. In another example, the nucleic acid construct comprises a sequence at least 95%, at least 96%, at least 97%, at least 98%, at least 99%, at least 99.5%, or 100% identical to SEQ ID NO: 731. In another example, the nucleic acid construct comprises a sequence at least 95%, at least 96%, at least 97%, at least 98%, at least 99%, at least 99.5%, or 100% identical to SEQ ID NO: 731 and encodes a multidomain therapeutic protein (or a multidomain therapeutic protein comprising a sequence) at least 99%, at least 99.5%, or 100% identical to SEQ ID NO: 572. In another example, the nucleic acid construct comprises a sequence at least 95%, at least 96%, at least 97%, at least 98%, at least 99%, at least 99.5%, or 100% identical to SEQ ID NO: 731 and encodes a multidomain therapeutic protein comprising the sequence set forth in SEQ ID NO: 572. In another example, the nucleic acid construct comprises a sequence at least 99%, at least 99.5%, or 100% identical to SEQ ID NO: 731. In another example, the nucleic acid construct comprises a sequence at least 99%, at least 99.5%, or 100% identical to SEQ ID NO: 731 and encodes a multidomain therapeutic protein (or a multidomain therapeutic protein comprising a sequence) at least 99%, at least 99.5%, or 100% identical to SEQ ID NO: 572. In another example, the nucleic acid construct comprises a sequence at least 99%, at least 99.5%, or 100% identical to SEQ ID NO: 731 and encodes a multidomain therapeutic protein comprising the sequence set forth in SEQ ID NO: 572. In another example, the nucleic acid construct comprises the sequence set forth in SEQ ID NO: 731. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the nucleic acid construct encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 572 (and, e.g., retaining the activity of native GAA). Optionally, the nucleic acid construct encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 572 (and, e.g., retaining the activity of native GAA). Optionally, the nucleic acid construct in the above examples encodes a multidomain therapeutic protein (or a multidomain therapeutic protein comprising a sequence) at least 99%, at least 99.5%, or 100% identical to SEQ ID NO: 572 (and, e.g., retaining the activity of native GAA). Optionally, the nucleic acid construct in the above examples encodes a multidomain therapeutic protein comprising the sequence set forth in SEQ ID NO: 572. Optionally, the nucleic acid construct in the above examples encodes a multidomain therapeutic protein consisting essentially of the sequence set forth in SEQ ID NO: 572. Optionally, the nucleic acid construct in the above examples encodes a multidomain therapeutic protein consisting of the sequence set forth in SEQ ID NO: 572.

Various multidomain therapeutic protein coding sequences are provided. In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 574 and 578-580. In another example, the multidomain therapeutic protein coding sequence is (or comprises a sequence) at least 95%, at least 96%, at least 97%, at least 98%, at least 99%, at least 99.5%, or 100% identical to any one of SEQ ID NOS: 574 and 578-580. In another example, the multidomain therapeutic protein coding sequence is (or comprises a sequence) at least 99%, at least 99.5%, or 100% identical to any one of SEQ ID NOS: 574 and 578-580. In another example, the multidomain therapeutic protein coding sequence comprises the sequence set forth in any one of SEQ ID NOS: 574 and 578-580. In another example, the multidomain therapeutic protein coding sequence consists essentially of the sequence set forth in any one of SEQ ID NOS: 574 and 578-580. In another example, the multidomain therapeutic protein coding sequence consists of the sequence set forth in any one of SEQ ID NOS: 574 and 578-580. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 570 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 570 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 570 (and, e.g., retaining the activity of native GAA). Optionally, the multidomain therapeutic protein coding sequence in the above examples encodes a multidomain therapeutic protein comprising the sequence set forth in SEQ ID NO: 570. Optionally, the multidomain therapeutic protein coding sequence in the above examples encodes a multidomain therapeutic protein consisting essentially of the sequence set forth in SEQ ID NO: 570. Optionally, the multidomain therapeutic protein coding sequence in the above examples encodes a multidomain therapeutic protein consisting of the sequence set forth in SEQ ID NO: 570.

Various codon optimized multidomain therapeutic protein coding sequences are provided. The multidomain therapeutic protein coding sequence can be, for example, CpG-depleted (e.g., fully CpG depleted) and/or codon optimized (e.g., CpG depleted (e.g., fully CpG-depleted) and codon optimized). In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 578-580. In another example, the multidomain therapeutic protein coding sequence is (or comprises a sequence) at least 95%, at least 96%, at least 97%, at least 98%, at least 99%, at least 99.5%, or 100% identical to any one of SEQ ID NOS: 578-580. In another example, the multidomain therapeutic protein coding sequence is (or comprises a sequence) at least 99%, at least 99.5%, or 100% identical to any one of SEQ ID NOS: 578-580. In another example, the multidomain therapeutic protein coding sequence comprises the sequence set forth in any one of SEQ ID NOS: 578-580. In another example, the multidomain therapeutic protein coding sequence consists essentially of the sequence set forth in any one of SEQ ID NOS: 578-580. In another example, the multidomain therapeutic protein coding sequence consists of the sequence set forth in any one of SEQ ID NOS: 578-580. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 570 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 570 (and, e.g., retaining the activity of native GAA). Optionally, the GAA coding sequence in the above examples encodes a multidomain therapeutic protein (or a multidomain therapeutic protein comprising a sequence) at least 99%, at least 99.5%, or 100% identical to SEQ ID NO: 570 (and, e.g., retaining the activity of native GAA). Optionally, the multidomain therapeutic protein coding sequence in the above examples encodes a multidomain therapeutic protein comprising the sequence set forth in SEQ ID NO: 570. Optionally, the multidomain therapeutic protein coding sequence in the above examples encodes a multidomain therapeutic protein consisting essentially of the sequence set forth in SEQ ID NO: 570. Optionally, the multidomain therapeutic protein coding sequence in the above examples encodes a multidomain therapeutic protein consisting of the sequence set forth in SEQ ID NO: 570.

In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 578. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 578 and encodes a multidomain therapeutic protein (or a multidomain therapeutic protein comprising a sequence) at least 99%, at least 99.5%, or 100% identical to SEQ ID NO: 570. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 578 and encodes a multidomain therapeutic protein comprising the sequence set forth in SEQ ID NO: 570. In another example, the multidomain therapeutic protein coding sequence is (or comprises a sequence) at least 95%, at least 96%, at least 97%, at least 98%, at least 99%, at least 99.5%, or 100% identical to SEQ ID NO: 578. In another example, the multidomain therapeutic protein coding sequence is (or comprises a sequence) at least 95%, at least 96%, at least 97%, at least 98%, at least 99%, at least 99.5%, or 100% identical to SEQ ID NO: 578 and encodes a multidomain therapeutic protein (or a multidomain therapeutic protein comprising a sequence) at least 99%, at least 99.5%, or 100% identical to SEQ ID NO: 570. In another example, the multidomain therapeutic protein coding sequence is (or comprises a sequence) at least 95%, at least 96%, at least 97%, at least 98%, at least 99%, at least 99.5%, or 100% identical to SEQ ID NO: 578 and encodes a multidomain therapeutic protein comprising the sequence set forth in SEQ ID NO: 570. In another example, the multidomain therapeutic protein coding sequence is (or comprises a sequence) at least 99%, at least 99.5%, or 100% identical to SEQ ID NO: 578. In another example, the multidomain therapeutic protein coding sequence is (or comprises a sequence) at least 99%, at least 99.5%, or 100% identical to SEQ ID NO: 578 and encodes a multidomain therapeutic protein (or a multidomain therapeutic protein comprising a sequence) at least 99%, at least 99.5%, or 100% identical to SEQ ID NO: 570. In another example, the multidomain therapeutic protein coding sequence is (or comprises a sequence) at least 99%, at least 99.5%, or 100% identical to SEQ ID NO: 578 and encodes a multidomain therapeutic protein comprising the sequence set forth in SEQ ID NO: 570. In another example, the multidomain therapeutic protein coding sequence comprises the sequence set forth in SEQ ID NO: 578. In another example, the multidomain therapeutic protein coding sequence consists essentially of the sequence set forth in SEQ ID NO: 578. In another example, the multidomain therapeutic protein coding sequence consists of the sequence set forth in SEQ ID NO: 578. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 570 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 570 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 570 (and, e.g., retaining the activity of native GAA). Optionally, the multidomain therapeutic protein coding sequence in the above examples encodes a multidomain therapeutic protein comprising the sequence set forth in SEQ ID NO: 570. Optionally, the multidomain therapeutic protein coding sequence in the above examples encodes a multidomain therapeutic protein consisting essentially of the sequence set forth in SEQ ID NO: 570. Optionally, the multidomain therapeutic protein coding sequence in the above examples encodes a multidomain therapeutic protein consisting of the sequence set forth in SEQ ID NO: 570.

In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 579. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 579 and encodes a multidomain therapeutic protein (or a multidomain therapeutic protein comprising a sequence) at least 99%, at least 99.5%, or 100% identical to SEQ ID NO: 570. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 579 and encodes a multidomain therapeutic protein comprising the sequence set forth in SEQ ID NO: 570. In another example, the multidomain therapeutic protein coding sequence is (or comprises a sequence) at least 95%, at least 96%, at least 97%, at least 98%, at least 99%, at least 99.5%, or 100% identical to SEQ ID NO: 579. In another example, the multidomain therapeutic protein coding sequence is (or comprises a sequence) at least 95%, at least 96%, at least 97%, at least 98%, at least 99%, at least 99.5%, or 100% identical to SEQ ID NO: 579 and encodes a multidomain therapeutic protein (or a multidomain therapeutic protein comprising a sequence) at least 99%, at least 99.5%, or 100% identical to SEQ ID NO: 570. In another example, the multidomain therapeutic protein coding sequence is (or comprises a sequence) at least 95%, at least 96%, at least 97%, at least 98%, at least 99%, at least 99.5%, or 100% identical to SEQ ID NO: 579 and encodes a multidomain therapeutic protein comprising the sequence set forth in SEQ ID NO: 570. In another example, the multidomain therapeutic protein coding sequence is (or comprises a sequence) at least 99%, at least 99.5%, or 100% identical to SEQ ID NO: 579. In another example, the multidomain therapeutic protein coding sequence is (or comprises a sequence) at least 99%, at least 99.5%, or 100% identical to SEQ ID NO: 579 and encodes a multidomain therapeutic protein (or a multidomain therapeutic protein comprising a sequence) at least 99%, at least 99.5%, or 100% identical to SEQ ID NO: 570. In another example, the multidomain therapeutic protein coding sequence is (or comprises a sequence) at least 99%, at least 99.5%, or 100% identical to SEQ ID NO: 579 and encodes a multidomain therapeutic protein comprising the sequence set forth in SEQ ID NO: 570. In another example, the multidomain therapeutic protein coding sequence comprises the sequence set forth in SEQ ID NO: 579. In another example, the multidomain therapeutic protein coding sequence consists essentially of the sequence set forth in SEQ ID NO: 579. In another example, the multidomain therapeutic protein coding sequence consists of the sequence set forth in SEQ ID NO: 579. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 570 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 570 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 570 (and, e.g., retaining the activity of native GAA). Optionally, the multidomain therapeutic protein coding sequence in the above examples encodes a multidomain therapeutic protein comprising the sequence set forth in SEQ ID NO: 570. Optionally, the multidomain therapeutic protein coding sequence in the above examples encodes a multidomain therapeutic protein consisting essentially of the sequence set forth in SEQ ID NO: 570. Optionally, the multidomain therapeutic protein coding sequence in the above examples encodes a multidomain therapeutic protein consisting of the sequence set forth in SEQ ID NO: 570.

In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 580. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 580 and encodes a multidomain therapeutic protein (or a multidomain therapeutic protein comprising a sequence) at least 99%, at least 99.5%, or 100% identical to SEQ ID NO: 570. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 580 and encodes a multidomain therapeutic protein comprising the sequence set forth in SEQ ID NO: 570. In another example, the multidomain therapeutic protein coding sequence is (or comprises a sequence) at least 95%, at least 96%, at least 97%, at least 98%, at least 99%, at least 99.5%, or 100% identical to SEQ ID NO: 580. In another example, the multidomain therapeutic protein coding sequence is (or comprises a sequence) at least 95%, at least 96%, at least 97%, at least 98%, at least 99%, at least 99.5%, or 100% identical to SEQ ID NO: 580 and encodes a multidomain therapeutic protein (or a multidomain therapeutic protein comprising a sequence) at least 99%, at least 99.5%, or 100% identical to SEQ ID NO: 570. In another example, the multidomain therapeutic protein coding sequence is (or comprises a sequence) at least 95%, at least 96%, at least 97%, at least 98%, at least 99%, at least 99.5%, or 100% identical to SEQ ID NO: 580 and encodes a multidomain therapeutic protein comprising the sequence set forth in SEQ ID NO: 570. In another example, the multidomain therapeutic protein coding sequence is (or comprises a sequence) at least 99%, at least 99.5%, or 100% identical to SEQ ID NO: 580. In another example, the multidomain therapeutic protein coding sequence is (or comprises a sequence) at least 99%, at least 99.5%, or 100% identical to SEQ ID NO: 580 and encodes a multidomain therapeutic protein (or a multidomain therapeutic protein comprising a sequence) at least 99%, at least 99.5%, or 100% identical to SEQ ID NO: 570. In another example, the multidomain therapeutic protein coding sequence is (or comprises a sequence) at least 99%, at least 99.5%, or 100% identical to SEQ ID NO: 580 and encodes a multidomain therapeutic protein comprising the sequence set forth in SEQ ID NO: 570. In another example, the multidomain therapeutic protein coding sequence comprises the sequence set forth in SEQ ID NO: 580. In another example, the multidomain therapeutic protein coding sequence consists essentially of the sequence set forth in SEQ ID NO: 580. In another example, the multidomain therapeutic protein coding sequence consists of the sequence set forth in SEQ ID NO: 580. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 570 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 570 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 570 (and, e.g., retaining the activity of native GAA). Optionally, the multidomain therapeutic protein coding sequence in the above examples encodes a multidomain therapeutic protein comprising the sequence set forth in SEQ ID NO: 570. Optionally, the multidomain therapeutic protein coding sequence in the above examples encodes a multidomain therapeutic protein consisting essentially of the sequence set forth in SEQ ID NO: 570. Optionally, the multidomain therapeutic protein coding sequence in the above examples encodes a multidomain therapeutic protein consisting of the sequence set forth in SEQ ID NO: 570.

Various multidomain therapeutic protein coding sequences are provided. In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 575. In another example, the multidomain therapeutic protein coding sequence is (or comprises a sequence) at least 95%, at least 96%, at least 97%, at least 98%, at least 99%, at least 99.5%, or 100% identical to SEQ ID NO: 575. In another example, the multidomain therapeutic protein coding sequence is (or comprises a sequence) at least 99%, at least 99.5%, or 100% identical to SEQ ID NO: 575. In another example, the multidomain therapeutic protein coding sequence comprises the sequence set forth in SEQ ID NO: 575. In another example, the multidomain therapeutic protein coding sequence consists essentially of the sequence set forth in SEQ ID NO: 575. In another example, the multidomain therapeutic protein coding sequence consists of the sequence set forth in SEQ ID NO: 575. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 571 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 571 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 571 (and, e.g., retaining the activity of native GAA). Optionally, the multidomain therapeutic protein coding sequence in the above examples encodes a multidomain therapeutic protein comprising the sequence set forth in SEQ ID NO: 571. Optionally, the multidomain therapeutic protein coding sequence in the above examples encodes a multidomain therapeutic protein consisting essentially of the sequence set forth in SEQ ID NO: 571. Optionally, the multidomain therapeutic protein coding sequence in the above examples encodes a multidomain therapeutic protein consisting of the sequence set forth in SEQ ID NO: 571.

When specific multidomain therapeutic protein nucleic acid constructs sequences are disclosed herein, they are meant to encompass the sequence disclosed or the reverse complement of the sequence. For example, if a multidomain therapeutic protein nucleic acid construct disclosed herein consists of the hypothetical sequence 5′-CTGGACCGA-3′, it is also meant to encompass the reverse complement of that sequence (5′-TCGGTCCAG-3′). Likewise, when construct elements are disclosed herein in a specific 5′ to 3′ order, they are also meant to encompass the reverse complement of the order of those elements. One reason for this is that, in many embodiments disclosed herein, the multidomain therapeutic protein nucleic acid constructs are part of a single-stranded recombinant AAV vector. Single-stranded AAV genomes are packaged as either sense (plus-stranded) or anti-sense (minus-stranded genomes), and single-stranded AAV genomes of + and - polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

### Vectors

The nucleic acid constructs disclosed herein can be provided in a vector for expression or for integration into and expression from a target genomic locus. A vector can comprise additional sequences such as, for example, replication origins, promoters, and genes encoding antibiotic resistance. A vector can also comprise nuclease agent components as disclosed elsewhere herein. For example, a vector can comprise a nucleic acid construct encoding a polypeptide of interest (e.g., encoding a multidomain therapeutic protein), a CRISPR/Cas system (nucleic acids encoding Cas protein and gRNA), one or more components of a CRISPR/Cas system, or a combination thereof (e.g., a nucleic acid construct and a gRNA). In some cases, a vector comprising a nucleic acid construct encoding a polypeptide of interest (e.g., encoding a multidomain therapeutic protein) does not comprise any components of the nuclease agents described herein (e.g., does not comprise a nucleic acid encoding a Cas protein and does not comprise a nucleic acid encoding a gRNA). Some such vectors comprise homology arms corresponding to target sites in the target genomic locus. Other such vectors do not comprise any homology arms.

Some vectors may be circular. Alternatively, the vector may be linear. The vector can be packaged for delivered via a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

The vectors can be, for example, viral vectors such as adeno-associated virus (AAV) vectors. The AAV may be any suitable serotype and may be a single-stranded AAV (ssAAV) or a self-complementary AAV (scAAV). Other exemplary viruses/viral vectors include retroviruses, lentiviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression or longer-lasting expression. Viral vector may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper virus to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging.

Exemplary viral titers (e.g., AAV titers) include about 10¹² to about 10¹⁶ vg/mL. Other exemplary viral titers (e.g., AAV titers) include about 10¹² to about 10¹⁶ vg/kg of body weight.

Adeno-associated viruses (AAVs) are endemic in multiple species including human and non-human primates (NHPs). At least 12 natural serotypes and hundreds of natural variants have been isolated and characterized to date. See, e.g., Li et al. (2020) Nat. Rev. Genet. 21:255-272, herein incorporated by reference in its entirety for all purposes. AAV particles are naturally composed of a non-enveloped icosahedral protein capsid containing a single-stranded DNA (ssDNA) genome. The DNA genome is flanked by two inverted terminal repeats (ITRs) which serve as the viral origins of replication and packaging signals. The rep gene encodes four proteins required for viral replication and packaging whilst the cap gene encodes the three structural capsid subunits which dictate the AAV serotype, and the Assembly Activating Protein (AAP) which promotes virion assembly in some serotypes.

Recombinant AAV (rAAV) is currently one of the most commonly used viral vectors used in gene therapy to treat human diseases by delivering therapeutic transgenes to target cells in vivo. Indeed, rAAV vectors are composed of icosahedral capsids similar to natural AAVs, but rAAV virions do not encapsidate AAV protein-coding or AAV replicating sequences. These viral vectors are non-replicating. The only viral sequences required in rAAV vectors are the two ITRs, which are needed to guide genome replication and packaging during manufacturing of the rAAV vector. rAAV genomes are devoid of AAV rep and cap genes, rendering them non-replicating in vivo. rAAV vectors are produced by expressing rep and cap genes along with additional viral helper proteins in trans, in combination with the intended transgene cassette flanked by AAV ITRs.

In therapeutic rAAV genomes, a gene expression cassette is placed between ITR sequences. Typically, rAAV genome cassettes comprise of a promoter to drive expression of a therapeutic transgene, followed by polyadenylation sequence. The ITRs flanking a rAAV expression cassette are usually derived from AAV2, the first serotype to be isolated and converted into a recombinant viral vector. Since then, most rAAV production methods rely on AAV2 Rep-based packaging systems. See, e.g., Colella et al. (2017) Mol. Ther. Methods Clin. Dev. 8:87-104, herein incorporated by reference in its entirety for all purposes.

Some non-limiting examples of ITRs that can be used include ITRs comprising, consisting essentially of, or consisting of SEQ ID NO: 158, SEQ ID NO: 159, or SEQ ID NO: 160. Other examples of ITRs comprise one or more mutations compared to SEQ ID NO: 158, SEQ ID NO: 159, or SEQ ID NO: 160 and can be at least 90%, at least 91%, at least 92%, at least 93%, at least 94%, at least 95%, at least 96%, at least 97%, at least 98%, or at least 99% identical to SEQ ID NO: 158, SEQ ID NO: 159, or SEQ ID NO: 160. In some rAAV genomes disclosed herein, the nucleic acid construct is flanked on both sides by the same ITR (i.e., the ITR on the 5′ end, and the reverse complement of the ITR on the 3′ end, such as SEQ ID NO: 158 on the 5′ end and SEQ ID NO: 168 on the 3′ end, or SEQ ID NO: 159 on the 5′ end and SEQ ID NO: 710 on the 3′ end, or SEQ ID NO: 160 on the 5′ end and SEQ ID NO: 711 on the 3′ end). In one example, the ITR on each end can comprise, consist essentially of, or consist of SEQ ID NO: 158 (i.e., SEQ ID NO: 158 on the 5′ end, and the reverse complement on the 3′ end). In another example, the ITR on each end can comprise, consist essentially of, or consist of SEQ ID NO: 159 (i.e., SEQ ID NO: 159 on the 5′ end, and the reverse complement on the 3′ end). In one example, the ITR on at least one end comprises, consists essentially of, or consists of SEQ ID NO: 160. In one example, the ITR on the 5′ end comprises, consists essentially of, or consists of SEQ ID NO: 160. In one example, the ITR on the 3′ end comprises, consists essentially of, or consists of SEQ ID NO: 160. In one example, the ITR on each end can comprise, consist essentially of, or consist of SEQ ID NO: 160 (i.e., SEQ ID NO: 160 on the 5′ end, and the reverse complement on the 3′ end). In other rAAV genomes disclosed herein, the nucleic acid construct is flanked by different ITRs on each end. In one example, the ITR on one end comprises, consists essentially of, or consists of SEQ ID NO: 158, and the ITR on the other end comprises, consists essentially of, or consists of SEQ ID NO: 159. In another example, the ITR on one end comprises, consists essentially of, or consists of SEQ ID NO: 158, and the ITR on the other end comprises, consists essentially of, or consists of SEQ ID NO: 160. In one example, the ITR on one end comprises, consists essentially of, or consists of SEQ ID NO: 159, and the ITR on the other end comprises, consists essentially of, or consists of SEQ ID NO: 160.

The specific serotype of a recombinant AAV vector influences its in vivo tropism to specific tissues. AAV capsid proteins are responsible for mediating attachment and entry into target cells, followed by endosomal escape and trafficking to the nucleus. Thus, the choice of serotype when developing a rAAV vector will influence what cell types and tissues the vector is most likely to bind to and transduce when injected in vivo. Several serotypes of rAAVs, including rAAV8, are capable of transducing the liver when delivered systemically in mice, NHPs and humans. See, e.g., Li et al. (2020) Nat. Rev. Genet. 21:255-272, herein incorporated by reference in its entirety for all purposes.

Once in the nucleus, the ssDNA genome is released from the virion and a complementary DNA strand is synthesized to generate a double-stranded DNA (dsDNA) molecule. Double-stranded AAV genomes naturally circularize via their ITRs and become episomes which will persist extrachromosomally in the nucleus. Therefore, for episomal gene therapy programs, rAAV-delivered rAAV episomes provide long-term, promoter-driven gene expression in non-dividing cells. However, this rAAV-delivered episomal DNA is diluted out as cells divide. In contrast, the gene therapy described herein is based on gene insertion to allow long-term gene expression.

When specific rAAVs comprising specific sequences (e.g., specific bidirectional construct sequences or specific unidirectional construct sequences) are disclosed herein, they are meant to encompass the sequence disclosed or the reverse complement of the sequence. For example, if a bidirectional or unidirectional construct disclosed herein consists of the hypothetical sequence 5′-CTGGACCGA-3′, it is also meant to encompass the reverse complement of that sequence (5′-TCGGTCCAG-3′). Likewise, when rAAVs comprising bidirectional or unidirectional construct elements in a specific 5′ to 3′ order are disclosed herein, they are also meant to encompass the reverse complement of the order of those elements. For example, if an rAAV is disclosed herein that comprises a bidirectional construct that comprises from 5′ to 3′ a first splice acceptor, a first coding sequence, a first terminator, a reverse complement of a second terminator, a reverse complement of a second coding sequence, and a reverse complement of a second splice acceptor, it is also meant to encompass a construct comprising from 5′ to 3′ the second splice acceptor, the second coding sequence, the second terminator, a reverse complement of the first terminator, a reverse complement of the first coding sequence, and a reverse complement of the first splice acceptor. Single-stranded AAV genomes are packaged as either sense (plus-stranded) or anti-sense (minus-stranded genomes), and single-stranded AAV genomes of + and - polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

The ssDNA AAV genome consists of two open reading frames, Rep and Cap, flanked by two inverted terminal repeats that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2a, and VA) mediate AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Multiple serotypes of AAV have been identified. These serotypes differ in the types of cells they infect (i.e., their tropism), allowing preferential transduction of specific cell types. The term AAV includes, for example, AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. An “AAV vector” as used herein refers to an AAV vector comprising a heterologous sequence not of AAV origin (i.e., a nucleic acid sequence heterologous to AAV), typically comprising a sequence encoding an exogenous polypeptide of interest. The construct may comprise an AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV capsid sequence. In general, the heterologous nucleic acid sequence (the transgene) is flanked by at least one, and generally by two, AAV inverted terminal repeat sequences (ITRs). An AAV vector may either be single-stranded (ssAAV) or self-complementary (scAAV). Examples of serotypes for liver tissue include AAV3B, AAV5, AAV6, AAV7, AAV8, AAV9, AAVrh.74, and AAVhu.37, and particularly AAV8. In a specific example, the AAV vector comprising the nucleic acid construct can be recombinant AAV8 (rAAV8). A rAAV8 vector as described herein is one in which the capsid is from AAV8. For example, an AAV vector using ITRs from AAV2 and a capsid of AAV8 is considered herein to be a rAAV8 vector.

Tropism can be further refined through pseudotyping, which is the mixing of a capsid and a genome from different viral serotypes. For example AAV⅖ indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y731F, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG.

To accelerate transgene expression, self-complementary AAV (scAAV) variants can be used. Because AAV depends on the cell’s DNA replication machinery to synthesize the complementary strand of the AAV’s single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

To increase packaging capacity, longer transgenes may be split between two AAV transfer plasmids, the first with a 3′ splice donor and the second with a 5′ splice acceptor. Upon co-infection of a cell, these viruses form concatemers, are spliced together, and the full-length transgene can be expressed. Although this allows for longer transgene expression, expression is less efficient. Similar methods for increasing capacity utilize homologous recombination. For example, a transgene can be divided between two transfer plasmids but with substantial sequence overlap such that co-expression induces homologous recombination and expression of the full-length transgene.

### B. Nuclease Agents and CRISPR/Cas Systems

The methods and compositions disclosed herein can utilize nuclease agents such as Clustered Regularly Interspersed Short Palindromic Repeats (CRISPR)/CRISPR-associated (Cas) systems, zinc finger nuclease (ZFN) systems, or Transcription Activator-Like Effector Nuclease (TALEN) systems or components of such systems to modify a target genomic locus in a target gene such as a safe harbor gene (e.g., ALB) for insertion of a nucleic acid construct as disclosed herein. Generally, the nuclease agents involve the use of engineered cleavage systems to induce a double strand break or a nick (i.e., a single strand break) in a nuclease target site. Cleavage or nicking can occur through the use of specific nucleases such as engineered ZFNs, TALENs, or CRISPR/Cas systems with an engineered guide RNA to guide specific cleavage or nicking of the nuclease target site. Any nuclease agent that induces a nick or double-strand break at a desired target sequence can be used in the methods and compositions disclosed herein. The nuclease agent can be used to create a site of insertion at a desired locus (target gene) within a host genome, at which site the nucleic acid construct is inserted to express the polypeptide of interest (e.g., multidomain therapeutic protein). The polypeptide of interest (e.g., multidomain therapeutic protein) may be exogenous with respect to its insertion site or locus (target gene), such as a safe harbor locus from which polypeptide of interest is not normally expressed. Alternatively, the polypeptide of interest may be non- exogenous with respect to its insertion site, such as insertion into an endogenous locus encoding the polypeptide of interest to correct a defective gene encoding the polypeptide of interest.

In one example, the nuclease agent is a CRISPR/Cas system. In another example, the nuclease agent comprises one or more ZFNs. In yet another example, the nuclease agent comprises one or more TALENs. In a specific example, the CRISPR/Cas systems or components of such systems target an ALB gene or locus (e.g., ALB genomic locus) within a cell, or intron 1 of an ALB gene or locus within a cell. In a more specific example, the CRISPR/Cas systems or components of such systems target a human ALB gene or locus or intron 1 of a human ALB gene or locus within a cell.

CRISPR/Cas systems include transcripts and other elements involved in the expression of, or directing the activity of, Cas genes. A CRISPR/Cas system can be, for example, a type I, a type II, a type III system, or a type V system (e.g., subtype V-A or subtype V-B). The methods and compositions disclosed herein can employ CRISPR/Cas systems by utilizing CRISPR complexes (comprising a guide RNA (gRNA) complexed with a Cas protein) for site-directed binding or cleavage of nucleic acids. A CRISPR/Cas system targeting an ALB gene or locus comprises a Cas protein (or a nucleic acid encoding the Cas protein) and one or more guide RNAs (or DNAs encoding the one or more guide RNAs), with each of the one or more guide RNAs targeting a different guide RNA target sequence in the target genomic locus (e.g., ALB gene or locus).

CRISPR/Cas systems used in the compositions and methods disclosed herein can be non-naturally occurring. A non-naturally occurring system includes anything indicating the involvement of the hand of man, such as one or more components of the system being altered or mutated from their naturally occurring state, being at least substantially free from at least one other component with which they are naturally associated in nature, or being associated with at least one other component with which they are not naturally associated. For example, some CRISPR/Cas systems employ non-naturally occurring CRISPR complexes comprising a gRNA and a Cas protein that do not naturally occur together, employ a Cas protein that does not occur naturally, or employ a gRNA that does not occur naturally.

### Target Genomic Loci and Albumin (ALB)

Any target genomic locus capable of expressing a gene can be used, such as a safe harbor locus (safe harbor gene, such as ALB) or an endogenous GAA locus. The nucleic acid construct can be integrated into any part of the target genomic locus. For example, the nucleic acid construct can be inserted into an intron or an exon of a target genomic locus or can replace one or more introns and/or exons of a target genomic locus. In a specific example, the nucleic acid construct can be integrated into an intron of the target genomic locus, such as the first intron of the target genomic locus (e.g., ALB intron 1). See, e.g., WO 2020/082042, US 2020/0270617, WO 2020/082041, US 2020/0268906, WO 2020/082046, and US 2020/0289628, each of which is herein incorporated by reference in its entirety for all purposes. Constructs integrated into a target genomic locus can be operably linked to an endogenous promoter at the target genomic locus (e.g., the endogenous ALB promoter).

Interactions between integrated exogenous DNA and a host genome can limit the reliability and safety of integration and can lead to overt phenotypic effects that are not due to the targeted genetic modification but are instead due to unintended effects of the integration on surrounding endogenous genes. For example, randomly inserted transgenes can be subject to position effects and silencing, making their expression unreliable and unpredictable. Likewise, integration of exogenous DNA into a chromosomal locus can affect surrounding endogenous genes and chromatin, thereby altering cell behavior and phenotypes. Safe harbor loci include chromosomal loci where transgenes or other exogenous nucleic acid inserts can be stably and reliably expressed in all tissues of interest without overtly altering cell behavior or phenotype (i.e., without any deleterious effects on the host cell). See, e.g., Sadelain et al. (2012) Nat. Rev. Cancer 12:51-58, herein incorporated by reference in its entirety for all purposes. For example, the safe harbor locus can be one in which expression of the inserted gene sequence is not perturbed by any read-through expression from neighboring genes. For example, safe harbor loci can include chromosomal loci where exogenous DNA can integrate and function in a predictable manner without adversely affecting endogenous gene structure or expression. Safe harbor loci can include extragenic regions or intragenic regions such as, for example, loci within genes that are non-essential, dispensable, or able to be disrupted without overt phenotypic consequences.

Such safe harbor loci can offer an open chromatin configuration in all tissues and can be ubiquitously expressed during embryonic development and in adults. See, e.g., Zambrowicz et al. (1997) Proc. Natl. Acad. Sci. U.S.A. 94:3789-3794, herein incorporated by reference in its entirety for all purposes. In addition, the safe harbor loci can be targeted with high efficiency, and safe harbor loci can be disrupted with no overt phenotype. Examples of safe harbor loci include ALB, CCR5, HPRT, AAVS1, and Rosa26. See, e.g., U.S. Pat. Nos. 7,888,121; 7,972,854; 7,914,796; 7,951,925; 8,110,379; 8,409,861; 8,586,526; and U.S. Pat. Publication Nos. 2003/0232410; 2005/0208489; 2005/0026157; 2006/0063231; 2008/0159996; 2010/00218264; 2012/0017290; 2011/0265198; 2013/0137104; 2013/0122591; 2013/0177983; 2013/0177960; and 2013/0122591, each of which is herein incorporated by reference in its entirety for all purposes. Other examples of target genomic loci include an ALB locus, a EESYR locus, a SARS locus, position 188,083,272 of human chromosome 1 or its non-human mammalian orthologue, position 3,046,320 of human chromosome 10 or its non-human mammalian orthologue, position 67, 328,980 of human chromosome 17 or its non-human mammalian orthologue, an adeno-associated virus site 1 (AAVS 1) on chromosome, a naturally occurring site of integration of AAV virus on human chromosome 19 or its non-human mammalian orthologue, a chemokine receptor 5 (CCR5) gene, a chemokine receptor gene encoding an HIV-1 coreceptor, or a mouse Rosa26 locus or its non-murine mammalian orthologue.

In a specific example, a safe harbor locus is a locus within the genome wherein a gene may be inserted without significant deleterious effects on the host cell such as a hepatocyte (e.g., without causing apoptosis, necrosis, and/or senescence, or without causing more than 5%, 10%, 15%, 20%, 25%, 30%, or 40% apoptosis, necrosis, and/or senescence as compared to a control population of cells). The safe harbor locus can allow overexpression of an exogenous gene without significant deleterious effects on the host cell such as a hepatocyte (e.g., without causing apoptosis, necrosis, and/or senescence, or without causing more than 5%, 10%, 15%, 20%, 25%, 30%, or 40% apoptosis, necrosis, and/or senescence as compared to a control population of cells). A desirable safe harbor locus may be one in which expression of the inserted gene sequence is not perturbed by read-through expression from neighboring genes. The safe harbor may be a human safe harbor (e.g., for a liver tissue or hepatocyte host cell).

In a specific example, the target genomic locus is an ALB locus, such as intron 1 of an ALB locus. In a more specific example, the target genomic locus is a human ALB locus, such as intron 1 of a human ALB locus (e.g., SEQ ID NO: 4).

### Cas Proteins

Cas proteins generally comprise at least one RNA recognition or binding domain that can interact with guide RNAs. Cas proteins can also comprise nuclease domains (e.g., DNase domains or RNase domains), DNA-binding domains, helicase domains, protein-protein interaction domains, dimerization domains, and other domains. Some such domains (e.g., DNase domains) can be from a native Cas protein. Other such domains can be added to make a modified Cas protein. A nuclease domain possesses catalytic activity for nucleic acid cleavage, which includes the breakage of the covalent bonds of a nucleic acid molecule. Cleavage can produce blunt ends or staggered ends, and it can be single-stranded or double-stranded. For example, a wild type Cas9 protein will typically create a blunt cleavage product. Alternatively, a wild type Cpf1 protein (e.g., FnCpf1) can result in a cleavage product with a 5-nucleotide 5′ overhang, with the cleavage occurring after the 18th base pair from the PAM sequence on the non-targeted strand and after the 23rd base on the targeted strand. A Cas protein can have full cleavage activity to create a double-strand break at a target genomic locus (e.g., a double-strand break with blunt ends), or it can be a nickase that creates a single-strand break at a target genomic locus.

Examples of Cas proteins include Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof.

An exemplary Cas protein is a Cas9 protein or a protein derived from a Cas9 protein. Cas9 proteins are from a type II CRISPR/Cas system and typically share four key motifs with a conserved architecture. Motifs 1, 2, and 4 are RuvC-like motifs, and motif 3 is an HNH motif. Exemplary Cas9 proteins are from Streptococcus pyogenes, Streptococcus thermophilus, Streptococcus sp., Staphylococcus aureus, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Neisseria meningitidis, or Campylobacter jejuni. Additional examples of the Cas9 family members are described in WO 2014/131833, herein incorporated by reference in its entirety for all purposes. Cas9 from S. pyogenes (SpCas9) (e.g., assigned UniProt accession number Q99ZW2) is an exemplary Cas9 protein. An exemplary SpCas9 protein sequence is set forth in SEQ ID NO: 8 (encoded by the DNA sequence set forth in SEQ ID NO: 9). An exemplary SpCas9 mRNA (cDNA) sequence is set forth in SEQ ID NO: 10. Smaller Cas9 proteins (e.g., Cas9 proteins whose coding sequences are compatible with the maximum AAV packaging capacity when combined with a guide RNA coding sequence and regulatory elements for the Cas9 and guide RNA, such as SaCas9 and CjCas9 and Nme2Cas9) are other exemplary Cas9 proteins. For example, Cas9 from S. aureus (SaCas9) (e.g., assigned UniProt accession number J7RUA5) is another exemplary Cas9 protein. Likewise, Cas9 from Campylobacter jejuni (CjCas9) (e.g., assigned UniProt accession number Q0P897) is another exemplary Cas9 protein. See, e.g., Kim et al. (2017) Nat. Commun. 8:14500, herein incorporated by reference in its entirety for all purposes. SaCas9 is smaller than SpCas9, and CjCas9 is smaller than both SaCas9 and SpCas9. Cas9 from Neisseria meningitidis (Nme2Cas9) is another exemplary Cas9 protein. See, e.g., Edraki et al. (2019) Mol. Cell 73(4):714-726, herein incorporated by reference in its entirety for all purposes. Cas9 proteins from Streptococcus thermophilus (e.g., Streptococcus thermophilus LMD-9 Cas9 encoded by the CRISPR1 locus (St1Cas9) or Streptococcus thermophilus Cas9 from the CRISPR3 locus (St3Cas9)) are other exemplary Cas9 proteins. Cas9 from Francisella novicida (FnCas9) or the RHA Francisella novicida Cas9 variant that recognizes an alternative PAM (E1369R/E1449H/R1556A substitutions) are other exemplary Cas9 proteins. These and other exemplary Cas9 proteins are reviewed, e.g., in Cebrian-Serrano and Davies (2017) Mamm. Genome 28(7):247-261, herein incorporated by reference in its entirety for all purposes. Examples of Cas9 coding sequences, Cas9 mRNAs, and Cas9 protein sequences are provided in WO 2013/176772, WO 2014/065596, WO 2016/106121, WO 2019/067910, WO 2020/082042, US 2020/0270617, WO 2020/082041, US 2020/0268906, WO 2020/082046, and US 2020/0289628, each of which is herein incorporated by reference in its entirety for all purposes. Specific examples of ORFs and Cas9 amino acid sequences are provided in Table 30 at paragraph [0449] WO 2019/067910, and specific examples of Cas9 mRNAs and ORFs are provided in paragraphs [0214]-[0234] of WO 2019/067910. See also WO 2020/082046 A2 (pp. 84-85) and Table 24 in WO 2020/069296, each of which is herein incorporated by reference in its entirety for all purposes. An exemplary SpCas9 protein sequence comprises, consists essentially of, or consists of SEQ ID NO: 11. An exemplary SpCas9 mRNA sequence encoding that SpCas9 protein sequence comprises, consists essentially of, or consists of SEQ ID NO: 12. Another exemplary SpCas9 mRNA sequence encoding that SpCas9 protein sequence comprises, consists essentially of, or consists of SEQ ID NO: 1. Another exemplary SpCas9 mRNA sequence encoding that SpCas9 protein sequence comprises SEQ ID NO: 2. An exemplary SpCas9 coding sequence comprises, consists essentially of, or consists of SEQ ID NO: 3.

Another example of a Cas protein is a Cpfl (CRISPR from Prevotella and Francisella 1) protein. Cpfl is a large protein (about 1300 amino acids) that contains a RuvC-like nuclease domain homologous to the corresponding domain of Cas9 along with a counterpart to the characteristic arginine-rich cluster of Cas9. However, Cpf1 lacks the HNH nuclease domain that is present in Cas9 proteins, and the RuvC-like domain is contiguous in the Cpfl sequence, in contrast to Cas9 where it contains long inserts including the HNH domain. See, e.g., Zetsche et al. (2015) Cell 163(3):759-771, herein incorporated by reference in its entirety for all purposes. Exemplary Cpf1 proteins are from Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, and Porphyromonas macacae. Cpfl from Francisella novicida U112 (FnCpf1; assigned UniProt accession number A0Q7Q2) is an exemplary Cpfl protein.

Another example of a Cas protein is CasX (Cas12e). CasX is an RNA-guided DNA endonuclease that generates a staggered double-strand break in DNA. CasX is less than 1000 amino acids in size. Exemplary CasX proteins are from Deltaproteobacteria (DpbCasX or DpbCas12e) and Planctomycetes (PlmCasX or PlmCas12e). Like Cpf1, CasX uses a single RuvC active site for DNA cleavage. See, e.g., Liu et al. (2019) Nature 566(7743):218-223, herein incorporated by reference in its entirety for all purposes.

Another example of a Cas protein is CasΦ (CasPhi or Cas12j), which is uniquely found in bacteriophages. CasΦ is less than 1000 amino acids in size (e.g., 700-800 amino acids). CasΦ cleavage generates staggered 5′ overhangs. A single RuvC active site in CasΦ is capable of crRNA processing and DNA cutting. See, e.g., Pausch et al. (2020) Science 369(6501):333-337, herein incorporated by reference in its entirety for all purposes.

Cas proteins can be wild type proteins (i.e., those that occur in nature), modified Cas proteins (i.e., Cas protein variants), or fragments of wild type or modified Cas proteins. Cas proteins can also be active variants or fragments with respect to catalytic activity of wild type or modified Cas proteins. Active variants or fragments with respect to catalytic activity can comprise at least 80%, 85%, 90%, 91%, 92%, 93%, 94%, 95%, 96%, 97%, 98%, 99% or more sequence identity to the wild type or modified Cas protein or a portion thereof, wherein the active variants retain the ability to cut at a desired cleavage site and hence retain nick-inducing or double-strand-break-inducing activity. Assays for nick-inducing or double-strand-break-inducing activity are known and generally measure the overall activity and specificity of the Cas protein on DNA substrates containing the cleavage site.

One example of a modified Cas protein is the modified SpCas9-HF1 protein, which is a high-fidelity variant of Streptococcus pyogenes Cas9 harboring alterations (N497A/R661A/Q695A/Q926A) designed to reduce non-specific DNA contacts. See, e.g., Kleinstiver et al. (2016) Nature 529(7587):490-495, herein incorporated by reference in its entirety for all purposes. Another example of a modified Cas protein is the modified eSpCas9 variant (K848A/K1003A/R1060A) designed to reduce off-target effects. See, e.g., Slaymaker et al. (2016) Science 351(6268):84-88, herein incorporated by reference in its entirety for all purposes. Other SpCas9 variants include K855A and K810A/K1003A/R1060A. These and other modified Cas proteins are reviewed, e.g., in Cebrian-Serrano and Davies (2017) Mamm. Genome 28(7):247-261, herein incorporated by reference in its entirety for all purposes. Another example of a modified Cas9 protein is xCas9, which is a SpCas9 variant that can recognize an expanded range of PAM sequences. See, e.g., Hu et al. (2018) Nature 556:57-63, herein incorporated by reference in its entirety for all purposes.

Cas proteins can be modified to increase or decrease one or more of nucleic acid binding affinity, nucleic acid binding specificity, and enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of or a property of the Cas protein.

Cas proteins can comprise at least one nuclease domain, such as a DNase domain. For example, a wild type Cpfl protein generally comprises a RuvC-like domain that cleaves both strands of target DNA, perhaps in a dimeric configuration. Likewise, CasX and CasΦ generally comprise a single RuvC-like domain that cleaves both strands of a target DNA. Cas proteins can also comprise at least two nuclease domains, such as DNase domains. For example, a wild type Cas9 protein generally comprises a RuvC-like nuclease domain and an HNH-like nuclease domain. The RuvC and HNH domains can each cut a different strand of double-stranded DNA to make a double-stranded break in the DNA. See, e.g., Jinek et al. (2012) Science 337(6096):816-821, herein incorporated by reference in its entirety for all purposes.

One or more of the nuclease domains can be deleted or mutated so that they are no longer functional or have reduced nuclease activity. For example, if one of the nuclease domains is deleted or mutated in a Cas9 protein, the resulting Cas9 protein can be referred to as a nickase and can generate a single-strand break within a double-stranded target DNA but not a double-strand break (i.e., it can cleave the complementary strand or the non-complementary strand, but not both). If none of the nuclease domains is deleted or mutated in a Cas9 protein, the Cas9 protein will retain double-strand-break-inducing activity. An example of a mutation that converts Cas9 into a nickase is a D10A (aspartate to alanine at position 10 of Cas9) mutation in the RuvC domain of Cas9 from S. pyogenes. Likewise, H939A (histidine to alanine at amino acid position 839), H840A (histidine to alanine at amino acid position 840), or N863A (asparagine to alanine at amino acid position N863) in the HNH domain of Cas9 from S. pyogenes can convert the Cas9 into a nickase. Other examples of mutations that convert Cas9 into a nickase include the corresponding mutations to Cas9 from S. thermophilus. See, e.g., Sapranauskas et al. (2011) Nucleic Acids Res. 39(21):9275-9282 and WO 2013/141680, each of which is herein incorporated by reference in its entirety for all purposes. Such mutations can be generated using methods such as site-directed mutagenesis, PCR-mediated mutagenesis, or total gene synthesis. Examples of other mutations creating nickases can be found, for example, in WO 2013/176772 and WO 2013/142578, each of which is herein incorporated by reference in its entirety for all purposes.

Examples of inactivating mutations in the catalytic domains of xCas9 are the same as those described above for SpCas9. Examples of inactivating mutations in the catalytic domains of Staphylococcus aureus Cas9 proteins are also known. For example, the Staphylococcus aureus Cas9 enzyme (SaCas9) may comprise a substitution at position N580 (e.g., N580A substitution) or a substitution at position D10 (e.g., D10A substitution) to generate a Cas nickase. See, e.g., WO 2016/106236, herein incorporated by reference in its entirety for all purposes. Examples of inactivating mutations in the catalytic domains of Nme2Cas9 are also known (e.g., D16A or H588A). Examples of inactivating mutations in the catalytic domains of St1Cas9 are also known (e.g., D9A, D598A, H599A, or N622A). Examples of inactivating mutations in the catalytic domains of St3Cas9 are also known (e.g., D10A or N870A). Examples of inactivating mutations in the catalytic domains of CjCas9 are also known (e.g., combination of D8A or H559A). Examples of inactivating mutations in the catalytic domains of FnCas9 and RHA FnCas9 are also known (e.g., N995A).

Examples of inactivating mutations in the catalytic domains of Cpf1 proteins are also known. With reference to Cpfl proteins from Francisella novicida U112 (FnCpf1), Acidaminococcus sp. BV3L6 (AsCpf1), Lachnospiraceae bacterium ND2006 (LbCpf1), and Moraxella bovoculi 237 (MbCpf1 Cpf1), such mutations can include mutations at positions 908, 993, or 1263 of AsCpf1 or corresponding positions in Cpf1 orthologs, or positions 832, 925, 947, or 1180 of LbCpf1 or corresponding positions in Cpf1 orthologs. Such mutations can include, for example one or more of mutations D908A, E993A, and D1263A of AsCpf1 or corresponding mutations in Cpf1 orthologs, or D832A, E925A, D947A, and D1180A of LbCpf1 or corresponding mutations in Cpf1 orthologs. See, e.g., US 2016/0208243, herein incorporated by reference in its entirety for all purposes.

Examples of inactivating mutations in the catalytic domains of CasX proteins are also known. With reference to CasX proteins from Deltaproteobacteria, D672A, E769A, and D935A (individually or in combination) or corresponding positions in other CasX orthologs are inactivating. See, e.g., Liu et al. (2019) Nature 566(7743):218-223, herein incorporated by reference in its entirety for all purposes.

Examples of inactivating mutations in the catalytic domains of CasΦ proteins are also known. For example, D371A and D394A, alone or in combination, are inactivating mutations. See, e.g., Pausch et al. (2020) Science 369(6501):333-337, herein incorporated by reference in its entirety for all purposes.

Cas proteins can also be operably linked to heterologous polypeptides as fusion proteins. For example, a Cas protein can be fused to a cleavage domain. See WO 2014/089290, herein incorporated by reference in its entirety for all purposesCas proteins can also be fused to a heterologous polypeptide providing increased or decreased stability. The fused domain or heterologous polypeptide can be located at the N-terminus, the C-terminus, or internally within the Cas protein.

As one example, a Cas protein can be fused to one or more heterologous polypeptides that provide for subcellular localization. Such heterologous polypeptides can include, for example, one or more nuclear localization signals (NLS) such as the monopartite SV40 NLS and/or a bipartite alpha-importin NLS for targeting to the nucleus, a mitochondrial localization signal for targeting to the mitochondria, an ER retention signal, and the like. See, e.g., Lange et al. (2007) J. Biol. Chem. 282(8):5101-5105, herein incorporated by reference in its entirety for all purposes. Such subcellular localization signals can be located at the N-terminus, the C-terminus, or anywhere within the Cas protein. An NLS can comprise a stretch of basic amino acids, and can be a monopartite sequence or a bipartite sequence. Optionally, a Cas protein can comprise two or more NLSs, including an NLS (e.g., an alpha-importin NLS or a monopartite NLS) at the N-terminus and an NLS (e.g., an SV40 NLS or a bipartite NLS) at the C-terminus. A Cas protein can also comprise two or more NLSs at the N-terminus and/or two or more NLSs at the C-terminus.

A Cas protein may, for example, be fused with 1-10 NLSs (e.g., fused with 1-5 NLSs or fused with one NLS. Where one NLS is used, the NLS may be linked at the N-terminus or the C-terminus of the Cas protein sequence. It may also be inserted within the Cas protein sequence. Alternatively, the Cas protein may be fused with more than one NLS. For example, the Cas protein may be fused with 2, 3, 4, or 5 NLSs. In a specific example, the Cas protein may be fused with two NLSs. In certain circumstances, the two NLSs may be the same (e.g., two SV40 NLSs) or different. For example, the Cas protein can be fused to two SV40 NLS sequences linked at the carboxy terminus. Alternatively, the Cas protein may be fused with two NLSs, one linked at the N-terminus and one at the C-terminus. In other examples, the Cas protein may be fused with 3 NLSs or with no NLS. The NLS may be a monopartite sequence, such as, e.g., the SV40 NLS, PKKKRKV (SEQ ID NO: 13) or PKKKRRV (SEQ ID NO: 14). The NLS may be a bipartite sequence, such as the NLS of nucleoplasmin, KRPAATKKAGQAKKKK (SEQ ID NO: 15). In a specific example, a single PKKKRKV (SEQ ID NO: 13) NLS may be linked at the C-terminus of the Cas protein. One or more linkers are optionally included at the fusion site.

Cas proteins can also be operably linked to a cell-penetrating domain or protein transduction domain. For example, the cell-penetrating domain can be derived from the HIV-1 TAT protein, the TLM cell-penetrating motif from human hepatitis B virus, MPG, Pep-1, VP22, a cell penetrating peptide from Herpes simplex virus, or a polyarginine peptide sequence. See, e.g., WO 2014/089290 and WO 2013/176772, each of which is herein incorporated by reference in its entirety for all purposes. The cell-penetrating domain can be located at the N-terminus, the C-terminus, or anywhere within the Cas protein.

Cas proteins can also be operably linked to a heterologous polypeptide for ease of tracking or purification, such as a fluorescent protein, a purification tag, or an epitope tag. Examples of fluorescent proteins include green fluorescent proteins (e.g., GFP, GFP-2, tagGFP, turboGFP, eGFP, Emerald, Azami Green, Monomeric Azami Green, CopGFP, AceGFP, ZsGreenl), yellow fluorescent proteins (e.g., YFP, eYFP, Citrine, Venus, YPet, PhiYFP, ZsYellowl), blue fluorescent proteins (e.g., eBFP, eBFP2, Azurite, mKalamal, GFPuv, Sapphire, T-sapphire), cyan fluorescent proteins (e.g., eCFP, Cerulean, CyPet, AmCyanl, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRedl, AsRed2, eqFP611, mRaspberry, mStrawberry, Jred), orange fluorescent proteins (e.g., mOrange, mKO, Kusabira-Orange, Monomeric Kusabira-Orange, mTangerine, tdTomato), and any other suitable fluorescent protein. Examples of tags include glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, hemagglutinin (HA), nus, Softag 1, Softag 3, Strep, SBP, Glu-Glu, HSV, KT3, S, S1, T7, V5, VSV-G, histidine (His), biotin carboxyl carrier protein (BCCP), and calmodulin.

Cas proteins can also be tethered to labeled nucleic acids. Such tethering (i.e., physical linking) can be achieved through covalent interactions or noncovalent interactions, and the tethering can be direct (e.g., through direct fusion or chemical conjugation, which can be achieved by modification of cysteine or lysine residues on the protein or intein modification), or can be achieved through one or more intervening linkers or adapter molecules such as streptavidin or aptamers. See, e.g., Pierce et al. (2005) Mini Rev. Med. Chem. 5(1):41-55; Duckworth et al. (2007) Angew. Chem. Int. Ed. Engl. 46(46):8819-8822; Schaeffer and Dixon (2009) Australian J. Chem. 62(10):1328-1332; Goodman et al. (2009) Chembiochem. 10(9):1551-1557; and Khatwani et al. (2012) Bioorg. Med. Chem. 20(14):4532-4539, each of which is herein incorporated by reference in its entirety for all purposes. Noncovalent strategies for synthesizing protein-nucleic acid conjugates include biotin-streptavidin and nickel-histidine methods. Covalent protein-nucleic acid conjugates can be synthesized by connecting appropriately functionalized nucleic acids and proteins using a wide variety of chemistries. Some of these chemistries involve direct attachment of the oligonucleotide to an amino acid residue on the protein surface (e.g., a lysine amine or a cysteine thiol), while other more complex schemes require post-translational modification of the protein or the involvement of a catalytic or reactive protein domain. Methods for covalent attachment of proteins to nucleic acids can include, for example, chemical cross-linking of oligonucleotides to protein lysine or cysteine residues, expressed protein-ligation, chemoenzymatic methods, and the use of photoaptamers. The labeled nucleic acid can be tethered to the C-terminus, the N-terminus, or to an internal region within the Cas protein. In one example, the labeled nucleic acid is tethered to the C-terminus or the N-terminus of the Cas protein. Likewise, the Cas protein can be tethered to the 5′ end, the 3′ end, or to an internal region within the labeled nucleic acid. That is, the labeled nucleic acid can be tethered in any orientation and polarity. For example, the Cas protein can be tethered to the 5′ end or the 3′ end of the labeled nucleic acid.

Cas proteins can be provided in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)) or DNA. Optionally, the nucleic acid encoding the Cas protein can be codon optimized for efficient translation into protein in a particular cell or organism. For example, the nucleic acid encoding the Cas protein can be modified to substitute codons having a higher frequency of usage in a bacterial cell, a yeast cell, a human cell, a non-human cell, a mammalian cell, a rodent cell, a mouse cell, a rat cell, or any other host cell of interest, as compared to the naturally occurring polynucleotide sequence. When a nucleic acid encoding the Cas protein is introduced into the cell, the Cas protein can be transiently, conditionally, or constitutively expressed in the cell.

Nucleic acids encoding Cas proteins can be stably integrated in the genome of a cell and operably linked to a promoter active in the cell. Alternatively, nucleic acids encoding Cas proteins can be operably linked to a promoter in an expression construct. Expression constructs include any nucleic acid constructs capable of directing expression of a gene or other nucleic acid sequence of interest (e.g., a Cas gene) and which can transfer such a nucleic acid sequence of interest to a target cell. For example, the nucleic acid encoding the Cas protein can be in a vector comprising a DNA encoding a gRNA. Alternatively, it can be in a vector or plasmid that is separate from the vector comprising the DNA encoding the gRNA. Promoters that can be used in an expression construct include promoters active, for example, in one or more of a eukaryotic cell, a human cell, a non-human cell, a mammalian cell, a non-human mammalian cell, a rodent cell, a mouse cell, a rat cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Optionally, the promoter can be a bidirectional promoter driving expression of both a Cas protein in one direction and a guide RNA in the other direction. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes. Use of a bidirectional promoter to express genes encoding a Cas protein and a guide RNA simultaneously allow for the generation of compact expression cassettes to facilitate delivery. In preferred embodiments, promotors are accepted by regulatory authorities for use in humans. In certain embodiments, promotors drive expression in a liver cell.

Different promoters can be used to drive Cas expression or Cas9 expression. In some methods, small promoters are used so that the Cas or Cas9 coding sequence can fit into an AAV construct. For example, Cas or Cas9 and one or more gRNAs (e.g., 1 gRNA or 2 gRNAs or 3 gRNAs or 4 gRNAs) can be delivered via LNP-mediated delivery (e.g., in the form of RNA) or adeno-associated virus (AAV)-mediated delivery (e.g., AAV2-mediated delivery, AAV5-mediated delivery, AAV8-mediated delivery, or AAV7m8-mediated delivery). For example, the nuclease agent can be CRISPR/Cas9, and a Cas9 mRNA and a gRNA targeting an intron 1 of an endogenous human ALB locus can be delivered via LNP-mediated delivery or AAV-mediated delivery. The Cas or Cas9 and the gRNA(s) can be delivered in a single AAV or via two separate AAVs. For example, a first AAV can carry a Cas or Cas9 expression cassette, and a second AAV can carry a gRNA expression cassette. Similarly, a first AAV can carry a Cas or Cas9 expression cassette, and a second AAV can carry two or more gRNA expression cassettes. Alternatively, a single AAV can carry a Cas or Cas9 expression cassette (e.g., Cas or Cas9 coding sequence operably linked to a promoter) and a gRNA expression cassette (e.g., gRNA coding sequence operably linked to a promoter). Similarly, a single AAV can carry a Cas or Cas9 expression cassette (e.g., Cas or Cas9 coding sequence operably linked to a promoter) and two or more gRNA expression cassettes (e.g., gRNA coding sequences operably linked to promoters). Different promoters can be used to drive expression of the gRNA, such as a U6 promoter or the small tRNA Gln. Likewise, different promoters can be used to drive Cas9 expression. For example, small promoters are used so that the Cas9 coding sequence can fit into an AAV construct. Similarly, small Cas9 proteins (e.g., SaCas9 or CjCas9 are used to maximize the AAV packaging capacity).

Cas proteins provided as mRNAs can be modified for improved stability and/or immunogenicity properties. The modifications may be made to one or more nucleosides within the mRNA. Examples of chemical modifications to mRNA nucleobases include pseudouridine, 1-methyl-pseudouridine, and 5-methyl-cytidine. mRNA encoding Cas proteins can also be capped. The cap can be, for example, a cap 1 structure in which the +1 ribonucleotide is methylated at the 2′O position of the ribose. The capping can, for example, give superior activity in vivo (e.g., by mimicking a natural cap), can result in a natural structure that reduce stimulation of the innate immune system of the host (e.g., can reduce activation of pattern recognition receptors in the innate immune system). mRNA encoding Cas proteins can also be polyadenylated (to comprise a poly(A) tail). mRNA encoding Cas proteins can also be modified to include pseudouridine (e.g., can be fully substituted with pseudouridine). As another example, capped and polyadenylated Cas mRNA containing N1-methyl-pseudouridine can be used. mRNA encoding Cas proteins can also be modified to include N1-methyl-pseudouridine (e.g., can be fully substituted with N1-methyl-pseudouridine). As another example, Cas mRNA fully substituted with pseudouridine can be used (i.e., all standard uracil residues are replaced with pseudouridine, a uridine isomer in which the uracil is attached with a carbon-carbon bond rather than nitrogen-carbon). As another example, Cas mRNA fully substituted with N1-methyl-pseudouridine can be used (i.e., all standard uracil residues are replaced with N1-methyl-pseudouridine). Likewise, Cas mRNAs can be modified by depletion of uridine using synonymous codons. For example, capped and polyadenylated Cas mRNA fully substituted with pseudouridine can be used. For example, capped and polyadenylated Cas mRNA fully substituted with N1-methyl-pseudouridine can be used.

Cas mRNAs can comprise a modified uridine at least at one, a plurality of, or all uridine positions. The modified uridine can be a uridine modified at the 5 position (e.g., with a halogen, methyl, or ethyl). The modified uridine can be a pseudouridine modified at the 1 position (e.g., with a halogen, methyl, or ethyl). The modified uridine can be, for example, pseudouridine, N1-methyl-pseudouridine, 5-methoxyuridine, 5-iodouridine, or a combination thereof. In some examples, the modified uridine is 5-methoxyuridine. In some examples, the modified uridine is 5-iodouridine. In some examples, the modified uridine is pseudouridine. In some examples, the modified uridine is N1-methyl-pseudouridine. In some examples, the modified uridine is a combination of pseudouridine and N1-methyl-pseudouridine. In some examples, the modified uridine is a combination of pseudouridine and 5-methoxyuridine. In some examples, the modified uridine is a combination of N1-methyl pseudouridine and 5-methoxyuridine. In some examples, the modified uridine is a combination of 5-iodouridine and N1-methyl-pseudouridine. In some examples, the modified uridine is a combination of pseudouridine and 5-iodouridine. In some examples, the modified uridine is a combination of 5-iodouridine and 5-methoxyuridine.

Cas mRNAs disclosed herein can also comprise a 5′ cap, such as a Cap0, Cap1, or Cap2. A 5′ cap is generally a 7-methylguanine ribonucleotide (which may be further modified, e.g., with respect to ARCA) linked through a 5′-triphosphate to the 5′ position of the first nucleotide of the 5′-to-3′ chain of the mRNA (i.e., the first cap-proximal nucleotide). In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc. Natl. Acad. Sci. U.S.A. 111(33):12025-30 and Abbas et al. (2017) Proc. Natl. Acad. Sci. U.S.A. 114(11):E2106-E2115, each of which is herein incorporated by reference in its entirety for all purposes. Most endogenous higher eukaryotic mRNAs, including mammalian mRNAs such as human mRNAs, comprise Cap1 or Cap2. Cap0 and other cap structures differing from Cap1 and Cap2 may be immunogenic in mammals, such as humans, due to recognition as non-self by components of the innate immune system such as IFIT-1 and IFIT-5, which can result in elevated cytokine levels including type I interferon. Components of the innate immune system such as IFIT-1 and IFIT-5 may also compete with eIF4E for binding of an mRNA with a cap other than Cap1 or Cap2, potentially inhibiting translation of the mRNA.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap in which the 2′ position of the first cap-proximal nucleotide is hydroxyl. See, e.g., Stepinski et al. (2001) RNA 7:1486-1495, herein incorporated by reference in its entirety for all purposes.

CleanCap^{Tm} AG (m7G(5′)ppp(5′)(2′OMeA)pG; TriLink Biotechnologies Cat. No. N-7113) or CleanCap^{Tm} GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively.

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give Cap0, in the presence of S-adenosyl methionine and GTP. See, e.g., Guo and Moss (1990) Proc. Natl. Acad. Sci. U.S.A. 87:4023-4027 and Mao and Shuman (1994) J. Biol. Chem. 269:24472-24479, each of which is herein incorporated by reference in its entirety for all purposes.

Cas mRNAs can further comprise a poly-adenylated (poly-A or poly(A) or poly-adenine) tail. The poly-A tail can, for example, comprise at least 20, at least 30, at least 40, at least 50, at least 60, at least 70, at least 80, at least 90, or at least 100 adenines, and optionally up to 300 adenines. For example, the poly-A tail can comprise 95, 96, 97, 98, 99, or 100 adenine nucleotides.

### Guide RNAs

A “guide RNA” or “gRNA” is an RNA molecule that binds to a Cas protein (e.g., Cas9 protein) and targets the Cas protein to a specific location within a target DNA. Guide RNAs can comprise two segments: a “DNA-targeting segment” (also called “guide sequence”) and a “protein-binding segment.” “Segment” includes a section or region of a molecule, such as a contiguous stretch of nucleotides in an RNA. Some gRNAs, such as those for Cas9, can comprise two separate RNA molecules: an “activator-RNA” (e.g., tracrRNA) and a “targeter-RNA” (e.g., CRISPR RNA or crRNA). Other gRNAs are a single RNA molecule (single RNA polynucleotide), which can also be called a “single-molecule gRNA,” a “single-guide RNA,” or an “sgRNA.” See, e.g., WO 2013/176772, WO 2014/065596, WO 2014/089290, WO 2014/093622, WO 2014/099750, WO 2013/142578, and WO 2014/131833, each of which is herein incorporated by reference in its entirety for all purposes. A guide RNA can refer to either a CRISPR RNA (crRNA) or the combination of a crRNA and a trans-activating CRISPR RNA (tracrRNA). The crRNA and tracrRNA can be associated as a single RNA molecule (single guide RNA or sgRNA) or in two separate RNA molecules (dual guide RNA or dgRNA). For Cas9, for example, a single-guide RNA can comprise a crRNA fused to a tracrRNA (e.g., via a linker). For Cpfl and CasΦ, for example, only a crRNA is needed to achieve binding to a target sequence. The terms “guide RNA” and “gRNA” include both double-molecule (i.e., modular) gRNAs and single-molecule gRNAs. In some of the methods and compositions disclosed herein, a gRNA is a S. pyogenes Cas9 gRNA or an equivalent thereof. In some of the methods and compositions disclosed herein, a gRNA is a S. aureus Cas9 gRNA or an equivalent thereof.

An exemplary two-molecule gRNA comprises a crRNA-like (“CRISPR RNA” or “targeter-RNA” or “crRNA” or “crRNA repeat”) molecule and a corresponding tracrRNA-like (“trans-activating CRISPR RNA” or “activator-RNA” or “tracrRNA”) molecule. A crRNA comprises both the DNA-targeting segment (single-stranded) of the gRNA and a stretch of nucleotides that forms one half of the dsRNA duplex of the protein-binding segment of the gRNA. An example of a crRNA tail (e.g., for use with S. pyogenes Cas9), located downstream (3′) of the DNA-targeting segment, comprises, consists essentially of, or consists of GUUUUAGAGCUAUGCU (SEQ ID NO: 16) or GUUUUAGAGCUAUGCUGUUUUG (SEQ ID NO: 17). Any of the DNA-targeting segments disclosed herein can be joined to the 5′ end of SEQ ID NO: 16 or 17 to form a crRNA.

A corresponding tracrRNA (activator-RNA) comprises a stretch of nucleotides that forms the other half of the dsRNA duplex of the protein-binding segment of the gRNA. A stretch of nucleotides of a crRNA are complementary to and hybridize with a stretch of nucleotides of a tracrRNA to form the dsRNA duplex of the protein-binding domain of the gRNA. As such, each crRNA can be said to have a corresponding tracrRNA. Examples of tracrRNA sequences (e.g., for use with S. pyogenes Cas9) comprise, consist essentially of, or consist of any one of

AGCAUAGCAAGUUAAAAUAAGGCUAGUCCGUUAUCAACUUGAAAAAGUGG
CACCGAGUCGGUGCUUU (SEQ ID NO: 18),

AAACAGCAUAGCAAGUUAAAAUAAGGCUAGUCCGUUAUCAACUUGAAAAA
GUGGCACCGAGUCGGUGCUUUU (SEQ ID NO: 19), or

GUUGGAACCAUUCAAAACAGCAUAGCAAGUUAAAAUAAGGCUAGUCCGUU
AUCAACUUGAAAAAGUGGCACCGAGUCGGUGC (SEQ ID NO: 20).

In systems in which both a crRNA and a tracrRNA are needed, the crRNA and the corresponding tracrRNA hybridize to form a gRNA. In systems in which only a crRNA is needed, the crRNA can be the gRNA. The crRNA additionally provides the single-stranded DNA-targeting segment that hybridizes to the complementary strand of a target DNA. If used for modification within a cell, the exact sequence of a given crRNA or tracrRNA molecule can be designed to be specific to the species in which the RNA molecules will be used. See, e.g., Mali et al. (2013) Science 339(6121):823-826; Jinek et al. (2012) Science 337(6096):816-821; Hwang et al. (2013) Nat. Biotechnol. 31(3):227-229; Jiang et al. (2013) Nat. Biotechnol. 31(3):233-239; and Cong et al. (2013) Science 339(6121):819-823, each of which is herein incorporated by reference in its entirety for all purposes.

The DNA-targeting segment (crRNA) of a given gRNA comprises a nucleotide sequence that is complementary to a sequence on the complementary strand of the target DNA, as described in more detail below. The DNA-targeting segment of a gRNA interacts with the target DNA in a sequence-specific manner via hybridization (i.e., base pairing). As such, the nucleotide sequence of the DNA-targeting segment may vary and determines the location within the target DNA with which the gRNA and the target DNA will interact. The DNA-targeting segment of a subject gRNA can be modified to hybridize to any desired sequence within a target DNA. Naturally occurring crRNAs differ depending on the CRISPR/Cas system and organism but often contain a targeting segment of between 21 to 72 nucleotides length, flanked by two direct repeats (DR) of a length of between 21 to 46 nucleotides (see, e.g., WO 2014/131833, herein incorporated by reference in its entirety for all purposes). In the case of S. pyogenes, the DRs are 36 nucleotides long and the targeting segment is 30 nucleotides long. The 3′ located DR is complementary to and hybridizes with the corresponding tracrRNA, which in turn binds to the Cas protein.

The DNA-targeting segment can have, for example, a length of at least about 12, at least about 15, at least about 17, at least about 18, at least about 19, at least about 20, at least about 25, at least about 30, at least about 35, or at least about 40 nucleotides. Such DNA-targeting segments can have, for example, a length from about 12 to about 100, from about 12 to about 80, from about 12 to about 50, from about 12 to about 40, from about 12 to about 30, from about 12 to about 25, or from about 12 to about 20 nucleotides. For example, the DNA targeting segment can be from about 15 to about 25 nucleotides (e.g., from about 17 to about 20 nucleotides, or about 17, 18, 19, or 20 nucleotides). See, e.g., US 2016/0024523, herein incorporated by reference in its entirety for all purposes. For Cas9 from S. pyogenes, a typical DNA-targeting segment is between 16 and 20 nucleotides in length or between 17 and 20 nucleotides in length. For Cas9 from S. aureus, a typical DNA-targeting segment is between 21 and 23 nucleotides in length. For Cpfl, a typical DNA-targeting segment is at least 16 nucleotides in length or at least 18 nucleotides in length.

In one example, the DNA-targeting segment can be about 20 nucleotides in length. However, shorter and longer sequences can also be used for the targeting segment (e.g., 15-25 nucleotides in length, such as 15, 16, 17, 18, 19, 20, 21, 22, 23, 24, or 25 nucleotides in length). The degree of identity between the DNA-targeting segment and the corresponding guide RNA target sequence (or degree of complementarity between the DNA-targeting segment and the other strand of the guide RNA target sequence) can be, for example, about 75%, about 80%, about 85%, about 90%, about 95%, or 100%. The DNA-targeting segment and the corresponding guide RNA target sequence can contain one or more mismatches. For example, the DNA-targeting segment of the guide RNA and the corresponding guide RNA target sequence can contain 1-4, 1-3, 1-2, 1, 2, 3, or 4 mismatches (e.g., where the total length of the guide RNA target sequence is at least 17, at least 18, at least 19, or at least 20 or more nucleotides). For example, the DNA-targeting segment of the guide RNA and the corresponding guide RNA target sequence can contain 1-4, 1-3, 1-2, 1, 2, 3, or 4 mismatches where the total length of the guide RNA target sequence 20 nucleotides.

As one example, a guide RNA targeting intron 1 of a human ALB gene can comprise a DNA-targeting segment (i.e., guide sequence) comprising, consisting essentially of, or consisting of the sequence (DNA-targeting segment) set forth in any one of SEQ ID NOS: 30-61. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 30-61. Alternatively, a guide RNA targeting intron 1 of a human ALB gene can comprise a DNA-targeting segment that is at least 75%, at least 80%, at least 85%, at least 90%, or at least 95% identical to the sequence (DNA-targeting segment) set forth in any one of SEQ ID NOS: 30-61. Alternatively, a guide RNA targeting intron 1 of a human ALB gene can comprise a DNA-targeting segment that is at least 90% or at least 95% identical to the sequence (DNA-targeting segment) set forth in any one of SEQ ID NOS: 30-61. Alternatively, a guide RNA targeting intron 1 of a human ALB gene can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 30-61. Alternatively, a guide RNA targeting intron 1 of a human ALB gene can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 30-61. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 30-61. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 30-61.

As another example, a guide RNA targeting intron 1 of a human ALB gene can comprise a DNA-targeting segment (i.e., guide sequence) comprising, consisting essentially of, or consisting of the sequence (DNA-targeting segment) set forth in any one of SEQ ID NOS: 36, 30, 33, and 41. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 36, 30, 33, and 41. Alternatively, a guide RNA targeting intron 1 of a human ALB gene can comprise a DNA-targeting segment that is at least 75%, at least 80%, at least 85%, at least 90%, or at least 95% identical to the sequence (DNA-targeting segment) set forth in any one of SEQ ID NOS: 36, 30, 33, and 41. Alternatively, a guide RNA targeting intron 1 of a human ALB gene can comprise a DNA-targeting segment that is at least 90% or at least 95% identical to the sequence (DNA-targeting segment) set forth in any one of SEQ ID NOS: 36, 30, 33, and 41. Alternatively, a guide RNA targeting intron 1 of a human ALB gene can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 36, 30, 33, and 41. Alternatively, a guide RNA targeting intron 1 of a human ALB gene can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 36, 30, 33, and 41. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 36, 30, 33, and 41. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 36, 30, 33, and 41.

As another example, a guide RNA targeting intron 1 of a human ALB gene can comprise a DNA-targeting segment (i.e., guide sequence) comprising, consisting essentially of, or consisting of the sequence (DNA-targeting segment) set forth in SEQ ID NO: 36. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 36. Alternatively, a guide RNA targeting intron 1 of a human ALB gene can comprise a DNA-targeting segment that is at least 75%, at least 80%, at least 85%, at least 90%, or at least 95% identical to the sequence (DNA-targeting segment) set forth in SEQ ID NO: 36. Alternatively, a guide RNA targeting intron 1 of a human ALB gene can comprise a DNA-targeting segment that is at least 90% or at least 95% identical to the sequence (DNA-targeting segment) set forth in SEQ ID NO: 36. Alternatively, a guide RNA targeting intron 1 of a human ALB gene can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 36. Alternatively, a guide RNA targeting intron 1 of a human ALB gene can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 36. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 36. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 36.

As another example, a guide RNA targeting intron 1 of a human ALB gene can comprise a DNA-targeting segment (i.e., guide sequence) comprising, consisting essentially of, or consisting of the sequence (DNA-targeting segment) set forth in SEQ ID NO: 30. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 30. Alternatively, a guide RNA targeting intron 1 of a human ALB gene can comprise a DNA-targeting segment that is at least 75%, at least 80%, at least 85%, at least 90%, or at least 95%identical to the sequence (DNA-targeting segment) set forth in SEQ ID NO: 30. Alternatively, a guide RNA targeting intron 1 of a human ALB gene can comprise a DNA-targeting segment that is at least 90% or at least 95% identical to the sequence (DNA-targeting segment) set forth in SEQ ID NO: 30. Alternatively, a guide RNA targeting intron 1 of a human ALB gene can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 30. Alternatively, a guide RNA targeting intron 1 of a human ALB gene can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 30. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 30. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 30.

As another example, a guide RNA targeting intron 1 of a human ALB gene can comprise a DNA-targeting segment (i.e., guide sequence) comprising, consisting essentially of, or consisting of the sequence (DNA-targeting segment) set forth in SEQ ID NO: 33. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 33. Alternatively, a guide RNA targeting intron 1 of a human ALB gene can comprise a DNA-targeting segment that is at least 75%, at least 80%, at least 85%, at least 90%, or at least 95% identical to the sequence (DNA-targeting segment) set forth in SEQ ID NO: 33. Alternatively, a guide RNA targeting intron 1 of a human ALB gene can comprise a DNA-targeting segment that is at least 90% or at least 95% identical to the sequence (DNA-targeting segment) set forth in SEQ ID NO: 33. Alternatively, a guide RNA targeting intron 1 of a human ALB gene can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 33. Alternatively, a guide RNA targeting intron 1 of a human ALB gene can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 33. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 33. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 33.

As another example, a guide RNA targeting intron 1 of a human ALB gene can comprise a DNA-targeting segment (i.e., guide sequence) comprising, consisting essentially of, or consisting of the sequence (DNA-targeting segment) set forth in SEQ ID NO: 41. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 41. Alternatively, a guide RNA targeting intron 1 of a human ALB gene can comprise a DNA-targeting segment that is at least 75%, at least 80%, at least 85%, at least 90%, or at least 95% identical to the sequence (DNA-targeting segment) set forth in SEQ ID NO: 41. Alternatively, a guide RNA targeting intron 1 of a human ALB gene can comprise a DNA-targeting segment that is at least 90% or at least 95% identical to the sequence (DNA-targeting segment) set forth in SEQ ID NO: 41. Alternatively, a guide RNA targeting intron 1 of a human ALB gene can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 41. Alternatively, a guide RNA targeting intron 1 of a human ALB gene can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 41. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 41. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 41.

TABLE 2

Human ALB Intron 1 Guide Sequences

| Guide Sequence | SEQ ID NO: |
|---|---|
| GAGCAACCUCACUCUUGUCU | 30 |
| AUGCAUUUGUUUCAAAAUAU | 31 |
| UGCAUUUGUUUCAAAAUAUU | 32 |
| AUUUAUGAGAUCAACAGCAC | 33 |
| GAUCAACAGCACAGGUUUUG | 34 |
| UUAAAUAAAGCAUAGUGCAA | 35 |
| UAAAGCAUAGUGCAAUGGAU | 36 |
| UAGUGCAAUGGAUAGGUCUU | 37 |
| UACUAAAACUUUAUUUUACU | 38 |
| AAAGUUGAACAAUAGAAAAA | 39 |
| AAUGCAUAAUCUAAGUCAAA | 40 |
| UAAUAAAAUUCAAACAUCCU | 41 |
| GCAUCUUUAAAGAAUUAUUU | 42 |
| UUUGGCAUUUAUUUCUAAAA | 43 |
| UGUAUUUGUGAAGUCUUACA | 44 |
| UCCUAGGUAAAAAAAAAAAA | 45 |
| UAAUUUUCUUUUGCGCACUA | 46 |
| UGACUGAAACUUCACAGAAU | 47 |
| GACUGAAACUUCACAGAAUA | 48 |
| UUCAUUUUAGUCUGUCUUCU | 49 |
| AUUAUCUAAGUUUGAAUAUA | 50 |
| AAUUUUUAAAAUAGUAUUCU | 51 |
| UGAAUUAUUCUUCUGUUUAA | 52 |
| AUCAUCCUGAGUUUUUCUGU | 53 |
| UUACUAAAACUUUAUUUUAC | 54 |
| ACCUUUUUUUUUUUUUACCU | 55 |
| AGUGCAAUGGAUAGGUCUUU | 56 |
| UGAUUCCUACAGAAAAACUC | 57 |
| UGGGCAAGGGAAGAAAAAAA | 58 |
| CCUCACUCUUGUCUGGGCAA | 59 |
| ACCUCACUCUUGUCUGGGCA | 60 |
| UGAGCAACCUCACUCUUGUC | 61 |

TABLE 3

Human ALB Intron 1 sgRNA Sequences.

| | |
|---|---|
| Full Sequence | Full Sequence Modified |
| GAGCAACCUCACUCUUGUCUGUUU UAGAGCUAGAAAUAGCAAGUUAAA AUAAGGCUAGUCCGUUAUCAACUU GAAAAAGUGGCACCGAGUCGGUGC UUUU (SEQ ID NO: 62) | mG^{∗}mA^{∗}mG^{∗}CAACCUCACUCUUGUCUGUUUUAGAmGmC mUmAmGmAmAmAmUmAmGmCAAGUUAAAAUAAGGCU AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 94) |
| AUGCAUUUGUUUCAAAAUAUGUUU UAGAGCUAGAAAUAGCAAGUUAAA AUAAGGCUAGUCCGUUAUCAACUU GAAAAAGUGGCACCGAGUCGGUGC UUUU (SEQ ID NO: 63) | mA^{∗}mU^{∗}mG^{∗}CAUUUGUUUCAAAAUAUGUUUUAGAmGmC mUmAmGmAmAmAmUmAmGmCAAGUUAAAAUAAGGCU AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 95) |
| UGCAUUUGUUUCAAAAUAUUGUUU UAGAGCUAGAAAUAGCAAGUUAAA AUAAGGCUAGUCCGUUAUCAACUU GAAAAAGUGGCACCGAGUCGGUGC UUUU (SEQ ID NO: 64) | mU^{∗}mG^{∗}mC^{∗}AUUUGUUUCAAAAUAUUGUUUUAGAmGmC mUmAmGmAmAmAmUmAmGmCAAGUUAAAAUAAGGCU AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 96) |
| AUUUAUGAGAUCAACAGCACGUUU UAGAGCUAGAAAUAGCAAGUUAAA AUAAGGCUAGUCCGUUAUCAACUU GAAAAAGUGGCACCGAGUCGGUGC UUUU (SEQ ID NO: 65) | mA^{∗}mU^{∗}mU^{∗}UAUGAGAUCAACAGCACGUUUUAGAmGmC mUmAmGmAmAmAmUmAmGmCAAGUUAAAAUAAGGCU AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 97) |
| GAUCAACAGCACAGGUUUUGGUUU UAGAGCUAGAAAUAGCAAGUUAAA AUAAGGCUAGUCCGUUAUCAACUU GAAAAAGUGGCACCGAGUCGGUGC UUUU (SEQ ID NO: 66) | mG^{∗}mA^{∗}mU^{∗}CAACAGCACAGGUUUUGGUUUUAGAmGmC mUmAmGmAmAmAmUmAmGmCAAGUUAAAAUAAGGCU AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 98) |
| UUAAAUAAAGCAUAGUGCAAGUUU UAGAGCUAGAAAUAGCAAGUUAAA AUAAGGCUAGUCCGUUAUCAACUU GAAAAAGUGGCACCGAGUCGGUGC UUUU (SEQ ID NO: 67) | mU^{∗}mU^{∗}mA^{∗}AAUAAAGCAUAGUGCAAGUUUUAGAmGmC mUmAmGmAmAmAmUmAmGmCAAGUUAAAAUAAGGCU AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 99) |
| UAAAGCAUAGUGCAAUGGAUGUUU UAGAGCUAGAAAUAGCAAGUUAAA AUAAGGCUAGUCCGUUAUCAACUU GAAAAAGUGGCACCGAGUCGGUGC UUUU (SEQ ID NO: 68) | mU^{∗}mA^{∗}mA^{∗}AGCAUAGUGCAAUGGAUGUUUUAGAmGmC mUmAmGmAmAmAmUmAmGmCAAGUUAAAAUAAGGCU AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 100) |
| UAGUGCAAUGGAUAGGUCUUGUUU UAGAGCUAGAAAUAGCAAGUUAAA AUAAGGCUAGUCCGUUAUCAACUU GAAAAAGUGGCACCGAGUCGGUGC UUUU (SEQ ID NO: 69) | mU^{∗}mA^{∗}mG^{∗}UGCAAUGGAUAGGUCUUGUUUUAGAmGmC mUmAmGmAmAmAmUmAmGmCAAGUUAAAAUAAGGCU AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 101) |
| UACUAAAACUUUAUUUUACUGUUU UAGAGCUAGAAAUAGCAAGUUAAA AUAAGGCUAGUCCGUUAUCAACUU GAAAAAGUGGCACCGAGUCGGUGC UUUU (SEQ ID NO: 70) | mU^{∗}mA^{∗}mC^{∗}UAAAACUUUAUUUUACUGUUUUAGAmGmC mUmAmGmAmAmAmUmAmGmCAAGUUAAAAUAAGGCU AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 102) |
| AAAGUUGAACAAUAGAAAAAGUUU UAGAGCUAGAAAUAGCAAGUUAAA AUAAGGCUAGUCCGUUAUCAACUU GAAAAAGUGGCACCGAGUCGGUGC UUUU (SEQ ID NO: 71) | mA^{∗}mA^{∗}mA^{∗}GUUGAACAAUAGAAAAAGUUUUAGAmGmC mUmAmGmAmAmAmUmAmGmCAAGUUAAAAUAAGGCU AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 103) |
| AAUGCAUAAUCUAAGUCAAAGUUU UAGAGCUAGAAAUAGCAAGUUAAA AUAAGGCUAGUCCGUUAUCAACUU GAAAAAGUGGCACCGAGUCGGUGC UUUU (SEQ ID NO: 72) | mA^{∗}mA^{∗}mU^{∗}GCAUAAUCUAAGUCAAAGUUUUAGAmGmC mUmAmGmAmAmAmUmAmGmCAAGUUAAAAUAAGGCU AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 104) |
| UAAUAAAAUUCAAACAUCCUGUUU UAGAGCUAGAAAUAGCAAGUUAAA AUAAGGCUAGUCCGUUAUCAACUU GAAAAAGUGGCACCGAGUCGGUGC UUUU (SEQ ID NO: 73) | mU^{∗}mA^{∗}mA^{∗}UAAAAUUCAAACAUCCUGUUUUAGAmGmC mUmAmGmAmAmAmUmAmGmCAAGUUAAAAUAAGGCU AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 105) |
| GCAUCUUUAAAGAAUUAUUUGUUU UAGAGCUAGAAAUAGCAAGUUAAA AUAAGGCUAGUCCGUUAUCAACUU GAAAAAGUGGCACCGAGUCGGUGC UUUU (SEQ ID NO: 74) | mG^{∗}mC^{∗}mA^{∗}UCUUUAAAGAAUUAUUUGUUUUAGAmGmC mUmAmGmAmAmAmUmAmGmCAAGUUAAAAUAAGGCU AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 106) |
| UUUGGCAUUUAUUUCUAAAAGUUU UAGAGCUAGAAAUAGCAAGUUAAA AUAAGGCUAGUCCGUUAUCAACUU | mU^{∗}mU^{∗}mU^{∗}GGCAUUUAUUUCUAAAAGUUUUAGAmGmC mUmAmGmAmAmAmUmAmGmCAAGUUAAAAUAAGGCU AGUCCGUUAUCAmAmCmUmUmGmAmAmAmAmAmGmU |
| GAAAAAGUGGCACCGAGUCGGUGC UUUU (SEQ ID NO: 75) | mGmGmCmAmCmCmGmAmGmUmCmGmGmUmGmCmU^{∗}m U^{∗}mU^{∗}mU (SEQ ID NO: 107) |
| UGUAUUUGUGAAGUCUUACAGUUU UAGAGCUAGAAAUAGCAAGUUAAA AUAAGGCUAGUCCGUUAUCAACUU GAAAAAGUGGCACCGAGUCGGUGC UUUU (SEQ ID NO: 76) | mU^{∗}mG^{∗}mU^{∗}AUUUGUGAAGUCUUACAGUUUUAGAmGmC mUmAmGmAmAmAmUmAmGmCAAGUUAAAAUAAGGCU AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 108) |
| UCCUAGGUAAAAAAAAAAAAGUUU UAGAGCUAGAAAUAGCAAGUUAAA AUAAGGCUAGUCCGUUAUCAACUU GAAAAAGUGGCACCGAGUCGGUGC UUUU (SEQ ID NO: 77) | mU^{∗}mC^{∗}mC^{∗}UAGGUAAAAAAAAAAAAGUUUUAGAmGmC mUmAmGmAmAmAmUmAmGmCAAGUUAAAAUAAGGCU AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 109) |
| UAAUUUUCUUUUGCGCACUAGUUU UAGAGCUAGAAAUAGCAAGUUAAA AUAAGGCUAGUCCGUUAUCAACUU GAAAAAGUGGCACCGAGUCGGUGC UUUU (SEQ ID NO: 78) | mU^{∗}mA^{∗}mA^{∗}UUUUCUUUUGCGCACUAGUUUUAGAmGmC mUmAmGmAmAmAmUmAmGmCAAGUUAAAAUAAGGCU AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 110) |
| UGACUGAAACUUCACAGAAUGUUU UAGAGCUAGAAAUAGCAAGUUAAA AUAAGGCUAGUCCGUUAUCAACUU GAAAAAGUGGCACCGAGUCGGUGC UUUU (SEQ ID NO: 79) | mU^{∗}mG^{∗}mA^{∗}CUGAAACUUCACAGAAUGUUUUAGAmGmC mUmAmGmAmAmAmUmAmGmCAAGUUAAAAUAAGGCU AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 111) |
| GACUGAAACUUCACAGAAUAGUUU UAGAGCUAGAAAUAGCAAGUUAAA AUAAGGCUAGUCCGUUAUCAACUU GAAAAAGUGGCACCGAGUCGGUGC UUUU (SEQ ID NO: 80) | mG^{∗}mA^{∗}mC^{∗}UGAAACUUCACAGAAUAGUUUUAGAmGmC mUmAmGmAmAmAmUmAmGmCAAGUUAAAAUAAGGCU AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 112) |
| UUCAUUUUAGUCUGUCUUCUGUUU UAGAGCUAGAAAUAGCAAGUUAAA AUAAGGCUAGUCCGUUAUCAACUU GAAAAAGUGGCACCGAGUCGGUGC UUUU (SEQ ID NO: 81) | mU^{∗}mU^{∗}mC^{∗}AUUUUAGUCUGUCUUCUGUUUUAGAmGmC mUmAmGmAmAmAmUmAmGmCAAGUUAAAAUAAGGCU AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 113) |
| AUUAUCUAAGUUUGAAUAUAGUUU UAGAGCUAGAAAUAGCAAGUUAAA AUAAGGCUAGUCCGUUAUCAACUU GAAAAAGUGGCACCGAGUCGGUGC UUUU (SEQ ID NO: 82) | mA^{∗}mU^{∗}mU^{∗}AUCUAAGUUUGAAUAUAGUUUUAGAmGmC mUmAmGmAmAmAmUmAmGmCAAGUUAAAAUAAGGCU AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 114) |
| AAUUUUUAAAAUAGUAUUCUGUUU UAGAGCUAGAAAUAGCAAGUUAAA AUAAGGCUAGUCCGUUAUCAACUU GAAAAAGUGGCACCGAGUCGGUGC UUUU (SEQ ID NO: 83) | mA^{∗}mA^{∗}mU^{∗}UUUUAAAAUAGUAUUCUGUUUUAGAmGmC mUmAmGmAmAmAmUmAmGmCAAGUUAAAAUAAGGCU AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 115) |
| UGAAUUAUUCUUCUGUUUAAGUUU UAGAGCUAGAAAUAGCAAGUUAAA AUAAGGCUAGUCCGUUAUCAACUU GAAAAAGUGGCACCGAGUCGGUGC UUUU (SEQ ID NO: 84) | mU^{∗}mG^{∗}mA^{∗}AUUAUUCUUCUGUUUAAGUUUUAGAmGmC mUmAmGmAmAmAmUmAmGmCAAGUUAAAAUAAGGCU AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 116) |
| AUCAUCCUGAGUUUUUCUGUGUUU UAGAGCUAGAAAUAGCAAGUUAAA AUAAGGCUAGUCCGUUAUCAACUU GAAAAAGUGGCACCGAGUCGGUGC UUUU (SEQ ID NO: 85) | mA^{∗}mU^{∗}mC^{∗}AUCCUGAGUUUUUCUGUGUUUUAGAmGmC mUmAmGmAmAmAmUmAmGmCAAGUUAAAAUAAGGCU AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 117) |
| UUACUAAAACUUUAUUUUACGUUU UAGAGCUAGAAAUAGCAAGUUAAA | mU^{∗}mU^{∗}mA^{∗}CUAAAACUUUAUUUUACGUUUUAGAmGmC mUmAmGmAmAmAmUmAmGmCAAGUUAAAAUAAGGCU |
| AUAAGGCUAGUCCGUUAUCAACUU GAAAAAGUGGCACCGAGUCGGUGC UUUU (SEQ ID NO: 86) | AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 118) |
| ACCUUUUUUUUUUUUUACCUGUUU UAGAGCUAGAAAUAGCAAGUUAAA AUAAGGCUAGUCCGUUAUCAACUU GAAAAAGUGGCACCGAGUCGGUGC UUUU (SEQ ID NO: 87) | mA^{∗}mC^{∗}mC^{∗}UUUUUUUUUUUUUACCUGUUUUAGAmGmC mUmAmGmAmAmAmUmAmGmCAAGUUAAAAUAAGGCU AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 119) |
| AGUGCAAUGGAUAGGUCUUUGUUU UAGAGCUAGAAAUAGCAAGUUAAA AUAAGGCUAGUCCGUUAUCAACUU GAAAAAGUGGCACCGAGUCGGUGC UUUU (SEQ ID NO: 88) | mA^{∗}mG^{∗}mU^{∗}GCAAUGGAUAGGUCUUUGUUUUAGAmGmC mUmAmGmAmAmAmUmAmGmCAAGUUAAAAUAAGGCU AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 120) |
| UGAUUCCUACAGAAAAACUCGUUU UAGAGCUAGAAAUAGCAAGUUAAA AUAAGGCUAGUCCGUUAUCAACUU GAAAAAGUGGCACCGAGUCGGUGC UUUU (SEQ ID NO: 89) | mU^{∗}mG^{∗}mA^{∗}UUCCUACAGAAAAACUCGUUUUAGAmGmC mUmAmGmAmAmAmUmAmGmCAAGUUAAAAUAAGGCU AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 121) |
| UGGGCAAGGGAAGAAAAAAAGUUU UAGAGCUAGAAAUAGCAAGUUAAA AUAAGGCUAGUCCGUUAUCAACUU GAAAAAGUGGCACCGAGUCGGUGC UUUU (SEQ ID NO: 90) | mU^{∗}mG^{∗}mG^{∗}GCAAGGGAAGAAAAAAAGUUUUAGAmGmC mUmAmGmAmAmAmUmAmGmCAAGUUAAAAUAAGGCU AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 122) |
| CCUCACUCUUGUCUGGGCAAGUUU UAGAGCUAGAAAUAGCAAGUUAAA AUAAGGCUAGUCCGUUAUCAACUU GAAAAAGUGGCACCGAGUCGGUGC UUUU (SEQ ID NO: 91) | mC^{∗}mC^{∗}mU^{∗}CACUCUUGUCUGGGCAAGUUUUAGAmGmC mUmAmGmAmAmAmUmAmGmCAAGUUAAAAUAAGGCU AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 123) |
| ACCUCACUCUUGUCUGGGCAGUUU UAGAGCUAGAAAUAGCAAGUUAAA AUAAGGCUAGUCCGUUAUCAACUU GAAAAAGUGGCACCGAGUCGGUGC UUUU (SEQ ID NO: 92) | mA^{∗}mC^{∗}mC^{∗}UCACUCUUGUCUGGGCAGUUUUAGAmGmC mUmAmGmAmAmAmUmAmGmCAAGUUAAAAUAAGGCU AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 124) |
| UGAGCAACCUCACUCUUGUCGUUU UAGAGCUAGAAAUAGCAAGUUAAA AUAAGGCUAGUCCGUUAUCAACUU GAAAAAGUGGCACCGAGUCGGUGC UUUU (SEQ ID NO: 93) | mU^{∗}mG^{∗}mA^{∗}GCAACCUCACUCUUGUCGUUUUAGAmGmC mUmAmGmAmAmAmUmAmGmCAAGUUAAAAUAAGGCU AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 125) |

TABLE 4

Mouse Alb Intron 1 Guide Sequences

| Guide Sequence | SEQ ID NO: |
|---|---|
| CACUCUUGUCUGUGGAAACA | 164 |

TABLE 5

Mouse Alb Intron 1 sgRNA Sequences

| | |
|---|---|
| Full Sequence | Full Sequence Modified |
| CACUCUUGUCUGUGGAAACAGUUU UAGAGCUAGAAAUAGCAAGUUAAA AUAAGGCUAGUCCGUUAUCAACUU GAAAAAGUGGCACCGAGUCGGUGC UUUU (SEQ ID NO: 166) | mC^{∗}mA^{∗}mC^{∗}UCUUGUCUGUGGAAACAGUUUUAGAmGmC mUmAmGmAmAmAmUmAmGmCAAGUUAAAAUAAGGCU AGUCCGUUAUCAmAmCmUmUmGmAmAmAmAmAmGmU mGmGmCmAmCmCmGmAmGmUmCmGmGmUmGmCmU^{∗}m U^{∗}mU^{∗}mU (SEQ ID NO: 167) |

TracrRNAs can be in any form (e.g., full-length tracrRNAs or active partial tracrRNAs) and of varying lengths. They can include primary transcripts or processed forms. For example, tracrRNAs (as part of a single-guide RNA or as a separate molecule as part of a two-molecule gRNA) may comprise, consist essentially of, or consist of all or a portion of a wild type tracrRNA sequence (e.g., about or more than about 20, 26, 32, 45, 48, 54, 63, 67, 85, or more nucleotides of a wild type tracrRNA sequence). Examples of wild type tracrRNA sequences from S. pyogenes include 171-nucleotide, 89-nucleotide, 75-nucleotide, and 65-nucleotide versions. See, e.g., Deltcheva et al. (2011) Nature 471(7340):602-607; WO 2014/093661, each of which is herein incorporated by reference in its entirety for all purposes. Examples of tracrRNAs within single-guide RNAs (sgRNAs) include the tracrRNA segments found within +48, +54, +67, and +85 versions of sgRNAs, where “+n” indicates that up to the +n nucleotide of wild type tracrRNA is included in the sgRNA. See US 8,697,359, herein incorporated by reference in its entirety for all purposes.

The percent complementarity between the DNA-targeting segment of the guide RNA and the complementary strand of the target DNA can be at least 60% (e.g., at least 65%, at least 70%, at least 75%, at least 80%, at least 85%, at least 90%, at least 95%, or 100%). The percent complementarity between the DNA-targeting segment and the complementary strand of the target DNA can be at least 60% over about 20 contiguous nucleotides. As an example, the percent complementarity between the DNA-targeting segment and the complementary strand of the target DNA can be 100% over the 14 contiguous nucleotides at the 5′ end of the complementary strand of the target DNA and as low as 0% over the remainder. In such a case, the DNA-targeting segment can be considered to be 14 nucleotides in length. As another example, the percent complementarity between the DNA-targeting segment and the complementary strand of the target DNA can be 100% over the seven contiguous nucleotides at the 5′ end of the complementary strand of the target DNA and as low as 0% over the remainder. In such a case, the DNA-targeting segment can be considered to be 7 nucleotides in length. In some guide RNAs, at least 17 nucleotides within the DNA-targeting segment are complementary to the complementary strand of the target DNA. For example, the DNA-targeting segment can be 20 nucleotides in length and can comprise 1, 2, or 3 mismatches with the complementary strand of the target DNA. In one example, the mismatches are not adjacent to the region of the complementary strand corresponding to the protospacer adjacent motif (PAM) sequence (i.e., the reverse complement of the PAM sequence) (e.g., the mismatches are in the 5′ end of the DNA-targeting segment of the guide RNA, or the mismatches are at least 2, 3, 4, 5, 6, 7, 8, 9, 10, 11, 12, 13, 14, 15, 16, 17, 18, or 19 base pairs away from the region of the complementary strand corresponding to the PAM sequence).

The protein-binding segment of a gRNA can comprise two stretches of nucleotides that are complementary to one another. The complementary nucleotides of the protein-binding segment hybridize to form a double-stranded RNA duplex (dsRNA). The protein-binding segment of a subject gRNA interacts with a Cas protein, and the gRNA directs the bound Cas protein to a specific nucleotide sequence within target DNA via the DNA-targeting segment.

Single-guide RNAs can comprise a DNA-targeting segment and a scaffold sequence (i.e., the protein-binding or Cas-binding sequence of the guide RNA). For example, such guide RNAs can have a 5′ DNA-targeting segment joined to a 3′ scaffold sequence. Exemplary scaffold sequences (e.g., for use with S. pyogenes Cas9) comprise, consist essentially of, or consist of:

GUUUUAGAGCUAGAAAUAGCAAGUUAAAAUAAGGCUAGUCCGUUAUCAAC
UUGAAAAAGUGGCACCGAGUCGGUGCU (version 1; SEQ ID NO:
21);

GUUGGAACCAUUCAAAACAGCAUAGCAAGUUAAAAUAAGGCUAGUCCGUU
AUCAACUUGAAAAAGUGGCACCGAGUCGGUGC (version 2; SEQ I
D NO: 22);

GUUUUAGAGCUAGAAAUAGCAAGUUAAAAUAAGGCUAGUCCGUUAUCAAC
UUGAAAAAGUGGCACCGAGUCGGUGC (version 3; SEQ ID NO:
23); and

GUUUAAGAGCUAUGCUGGAAACAGCAUAGCAAGUUUAAAUAAGGCUAGUC
CGUUAUCAACUUGAAAAAGUGGCACCGAGUCGGUGC (version 4; S
EQ ID NO: 24);

GUUUUAGAGCUAGAAAUAGCAAGUUAAAAUAAGGCUAGUCCGUUAUCAAC
UUGAAAAAGUGGCACCGAGUCGGUGCUUUUUUU (version 5; SEQ
ID NO: 25);

GUUUUAGAGCUAGAAAUAGCAAGUUAAAAUAAGGCUAGUCCGUUAUCAAC
UUGAAAAAGUGGCACCGAGUCGGUGCUUUU (version 6; SEQ ID
NO: 26);

GUUUAAGAGCUAUGCUGGAAACAGCAUAGCAAGUUUAAAUAAGGCUAGUC
CGUUAUCAACUUGAAAAAGUGGCACCGAGUCGGUGCUUUUUU (versio
n 7; SEQ ID NO: 27);or

GUUUUAGAGCUAGAAAUAGCAAGUUAAAAUAAGGCUAGUCCGUUAUCAAC
UUGGCACCGAGUCGGUGC (version 8; SEQ ID NO: 28).

In some guide sgRNAs, the four terminalU residues of version 6 are not present. In some sgRNAs, only 1, 2, or 3 of the four terminal U residues of version 6 are present. Guide RNAs targeting any of the guide RNA target sequences disclosed herein can include, for example, a DNA-targeting segment on the 5′ end of the guide RNA fused to any of the exemplary guide RNA scaffold sequences on the 3′ end of the guide RNA. That is, any of the DNA-targeting segments disclosed herein can be joined to the 5′ end of any one of the above scaffold sequences to form a single guide RNA (chimeric guide RNA).

Guide RNAs can include modifications or sequences that provide for additional desirable features (e.g., modified or regulated stability; subcellular targeting; tracking with a fluorescent label; a binding site for a protein or protein complex; and the like). That is, guide RNAs can include one or more modified nucleosides or nucleotides, or one or more non-naturally and/or naturally occurring components or configurations that are used instead of or in addition to the canonical A, G, C, and U residues. Examples of such modifications include, for example, a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, and so forth); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof. Other examples of modifications include engineered stem loop duplex structures, engineered bulge regions, engineered hairpins 3′ of the stem loop duplex structure, or any combination thereof. See, e.g., US 2015/0376586, herein incorporated by reference in its entirety for all purposes. A bulge can be an unpaired region of nucleotides within the duplex made up of the crRNA-like region and the minimum tracrRNA-like region. A bulge can comprise, on one side of the duplex, an unpaired 5′-XXXY-3′ where X is any purine and Y can be a nucleotide that can form a wobble pair with a nucleotide on the opposite strand, and an unpaired nucleotide region on the other side of the duplex.

Guide RNAs can comprise modified nucleosides and modified nucleotides including, for example, one or more of the following: (1) alteration or replacement of one or both of the non-linking phosphate oxygens and/or of one or more of the linking phosphate oxygens in the phosphodiester backbone linkage (an exemplary backbone modification); (2) alteration or replacement of a constituent of the ribose sugar such as alteration or replacement of the 2′ hydroxyl on the ribose sugar (an exemplary sugar modification); (3) replacement (e.g., wholesale replacement) of the phosphate moiety with dephospho linkers (an exemplary backbone modification); (4) modification or replacement of a naturally occurring nucleobase, including with a non-canonical nucleobase (an exemplary base modification); (5) replacement or modification of the ribose-phosphate backbone (an exemplary backbone modification); (6) modification of the 3′ end or 5′ end of the oligonucleotide (e.g., removal, modification or replacement of a terminal phosphate group or conjugation of a moiety, cap, or linker (such 3′ or 5′ cap modifications may comprise a sugar and/or backbone modification); and (7) modification or replacement of the sugar (an exemplary sugar modification). Other possible guide RNA modifications include modifications of or replacement of uracils or poly-uracil tracts. See, e.g., WO 2015/048577 and US 2016/0237455, each of which is herein incorporated by reference in its entirety for all purposes. Similar modifications can be made to Cas-encoding nucleic acids, such as Cas mRNAs. For example, Cas mRNAs can be modified by depletion of uridine using synonymous codons.

Chemical modifications such at hose listed above can be combined to provide modified gRNAs and/or mRNAs comprising residues (nucleosides and nucleotides) that can have two, three, four, or more modifications. For example, a modified residue can have a modified sugar and a modified nucleobase. In one example, every base of a gRNA is modified (e.g., all bases have a modified phosphate group, such as a phosphorothioate group). For example, all or substantially all of the phosphate groups of a gRNA can be replaced with phosphorothioate groups. Alternatively or additionally, a modified gRNA can comprise at least one modified residue at or near the 5′ end. Alternatively or additionally, a modified gRNA can comprise at least one modified residue at or near the 3′ end.

Some gRNAs comprise one, two, three or more modified residues. For example, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of the positions in a modified gRNA can be modified nucleosides or nucleotides.

Unmodified nucleic acids can be prone to degradation. Exogenous nucleic acids can also induce an innate immune response. Modifications can help introduce stability and reduce immunogenicity. Some gRNAs described herein can contain one or more modified nucleosides or nucleotides to introduce stability toward intracellular or serum-based nucleases. Some modified gRNAs described herein can exhibit a reduced innate immune response when introduced into a population of cells.

The gRNAs disclosed herein can comprise a backbone modification in which the phosphate group of a modified residue can be modified by replacing one or more of the oxygens with a different substituent. The modification can include the wholesale replacement of an unmodified phosphate moiety with a modified phosphate group as described herein. Backbone modifications of the phosphate backbone can also include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (Rp) or the “S” configuration (Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group (a sugar modification). For example, the 2′ hydroxyl group (OH) can be modified (e.g., replaced with a number of different oxy or deoxy substituents. Modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). The 2′ hydroxyl group modification can be 2′-O-Me. Likewise, the 2′ hydroxyl group modification can be a 2′-fluoro modification, which replaces the 2′ hydroxyl group with a fluoride. The 2′ hydroxyl group modification can include locked nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). The 2′ hydroxyl group modification can include unlocked nucleic acids (UNA) in which the ribose ring lacks the C2′-C3’ bond. The 2′ hydroxyl group modification can include the methoxyethyl group (MOE), (OCH₂CH₂OCH₃, e.g., a PEG derivative).

Deoxy 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH2; alkylamino, dialkylamino, heterocyclyl, arylamino, diarylamino, heteroarylamino, diheteroarylamino, or amino acid); NH(CH₂CH₂NH)ₙCH₂CH₂-amino (wherein amino can be, e.g., as described herein), -NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form (e.g. L-nucleosides).

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In a dual guide RNA, each of the crRNA and the tracrRNA can contain modifications. Such modifications may be at one or both ends of the crRNA and/or tracrRNA. In a sgRNA, one or more residues at one or both ends of the sgRNA may be chemically modified, and/or internal nucleosides may be modified, and/or the entire sgRNA may be chemically modified. Some gRNAs comprise a 5′ end modification. Some gRNAs comprise a 3′ end modification.

The guide RNAs disclosed herein can comprise one of the modification patterns disclosed in WO 2018/107028 A1, herein incorporated by reference in its entirety for all purposes. The guide RNAs disclosed herein can also comprise one of the structures/modification patterns disclosed in US 2017/0114334, herein incorporated by reference in its entirety for all purposes. The guide RNAs disclosed herein can also comprise one of the structures/modification patterns disclosed in WO 2017/136794, WO 2017/004279, US 2018/0187186, or US 2019/0048338, each of which is herein incorporated by reference in its entirety for all purposes.

As one example, nucleotides at the 5′ or 3′ end of a guide RNA can include phosphorothioate linkages (e.g., the bases can have a modified phosphate group that is a phosphorothioate group). For example, a guide RNA can include phosphorothioate linkages between the 2, 3, or 4 terminal nucleotides at the 5′ or 3′ end of the guide RNA. As another example, nucleotides at the 5′ and/or 3′ end of a guide RNA can have 2′-O-methyl modifications. For example, a guide RNA can include 2′-O-methyl modifications at the 2, 3, or 4 terminal nucleotides at the 5′ and/or 3′ end of the guide RNA (e.g., the 5′ end). See, e.g., WO 2017/173054 A1 and Finn et al. (2018) Cell Rep. 22(9):2227-2235, each of which is herein incorporated by reference in its entirety for all purposes. Other possible modifications are described in more detail elsewhere herein. In a specific example, a guide RNA includes 2′-O-methyl analogs and 3′ phosphorothioate internucleotide linkages at the first three 5′ and 3′ terminal RNA residues. Such chemical modifications can, for example, provide greater stability and protection from exonucleases to guide RNAs, allowing them to persist within cells for longer than unmodified guide RNAs. Such chemical modifications can also, for example, protect against innate intracellular immune responses that can actively degrade RNA or trigger immune cascades that lead to cell death.

As one example, any of the guide RNAs described herein can comprise at least one modification. In one example, the at least one modification comprises a 2′-O-methyl (2′-O-Me) modified nucleotide, a phosphorothioate (PS) bond between nucleotides, a 2′-fluoro (2′-F) modified nucleotide, or a combination thereof. For example, the at least one modification can comprise a 2′-O-methyl (2′-O-Me) modified nucleotide. Alternatively or additionally, the at least one modification can comprise a phosphorothioate (PS) bond between nucleotides. Alternatively or additionally, the at least one modification can comprise a 2′-fluoro (2′-F) modified nucleotide. In one example, a guide RNA described herein comprises one or more 2′-O-methyl (2′-O-Me) modified nucleotides and one or more phosphorothioate (PS) bonds between nucleotides.

The modifications can occur anywhere in the guide RNA. As one example, the guide RNA comprises a modification at one or more of the first five nucleotides at the 5′ end of the guide RNA, the guide RNA comprises a modification at one or more of the last five nucleotides of the 3′ end of the guide RNA, or a combination thereof. For example, the guide RNA can comprise phosphorothioate bonds between the first four nucleotides of the guide RNA, phosphorothioate bonds between the last four nucleotides of the guide RNA, or a combination thereof. Alternatively or additionally, the guide RNA can comprise 2′-O-Me modified nucleotides at the first three nucleotides at the 5′ end of the guide RNA, can comprise 2′-O-Me modified nucleotides at the last three nucleotides at the 3′ end of the guide RNA, or a combination thereof.

In one example, a modified gRNA can comprise the following sequence: mN^{∗}mN^{∗}mN^{∗}NNNNNNNNNNNNNNNNNGUUUUAGAmGmCmUmAmGmAmAmAmUmA mGmCAAGUUAAAAUAAGGCUAGUCCGUUAUCAmAmCmUmUmGmAmAmAmAmAm GmUmGmGmCmAmCmCmGmAmGmUmCmGmGmUmGmCmU^{∗}mU^{∗}mU^{∗}mU (SEQ ID NO: 29), where “N” may be any natural or non-natural nucleotide. For example, the totality of N residues comprise a human ALB intron 1 DNA-targeting segment as described herein (e.g., the sequence set forth in SEQ ID NO: 29, wherein the N residues are replaced with the DNA-targeting segment of any one of SEQ ID NOS: 30-61, the DNA-targeting segment of any one of SEQ ID NOS: 36, 30, 33, and 41, or the DNA-targeting segment of SEQ ID NO: 36. For example, a modified gRNA can comprise the sequence set forth in any one of SEQ ID NOS: 94-125, the sequence set forth in any one of SEQ ID NOS: 100, 94, 97, and 105, or the sequence set forth in SEQ ID NO: 100 in Table 3. The terms “mA,” “mC,” “mU,” and “mG” denote a nucleotide (A, C, U, and G, respectively) that has been modified with 2′-O-Me. The symbol “^{∗}” depicts a phosphorothioate modification. In certain embodiments, A, C, G, U, and N independently denote a ribose sugar, i.e., 2′-OH. In certain embodiments in the context of a modified sequence, A, C, G, U, and N denote a ribose sugar, i.e., 2′-OH. A phosphorothioate linkage or bond refers to a bond where a sulfur is substituted for one nonbridging phosphate oxygen in a phosphodiester linkage, for example in the bonds between nucleotides bases. When phosphorothioates are used to generate oligonucleotides, the modified oligonucleotides may also be referred to as S-oligos. The terms A^{∗}, C^{∗}, U^{∗}, or G^{∗} denote a nucleotide that is linked to the next (e.g., 3′) nucleotide with a phosphorothioate bond. The terms “mA^{∗},” “mC^{∗},” “mU^{∗},” and “mG^{∗}” denote a nucleotide (A, C, U, and G, respectively) that has been substituted with 2′-O-Me and that is linked to the next (e.g., 3′) nucleotide with a phosphorothioate bond.

Another chemical modification that has been shown to influence nucleotide sugar rings is halogen substitution. For example, 2′-fluoro (2′-F) substitution on nucleotide sugar rings can increase oligonucleotide binding affinity and nuclease stability. Abasic nucleotides refer to those which lack nitrogenous bases. Inverted bases refer to those with linkages that are inverted from the normal 5′ to 3′ linkage (i.e., either a 5′ to 5′ linkage or a 3′ to 3′ linkage).

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In one example, one or more of the first three, four, or five nucleotides at the 5′ terminus, and one or more of the last three, four, or five nucleotides at the 3′ terminus are modified. The modification can be, for example, a 2′-O-Me, 2′-F, inverted abasic nucleotide, phosphorothioate bond, or other nucleotide modification well known to increase stability and/or performance.

In another example, the first four nucleotides at the 5′ terminus, and the last four nucleotides at the 3′ terminus can be linked with phosphorothioate bonds.

In another example, the first three nucleotides at the 5′ terminus, and the last three nucleotides at the 3′ terminus can comprise a 2′-O-methyl (2′-O-Me) modified nucleotide. In another example, the first three nucleotides at the 5′ terminus, and the last three nucleotides at the 3′ terminus comprise a 2′-fluoro (2′-F) modified nucleotide. In another example, the first three nucleotides at the 5′ terminus, and the last three nucleotides at the 3′ terminus comprise an inverted abasic nucleotide.

Guide RNAs can be provided in any form. For example, the gRNA can be provided in the form of RNA, either as two molecules (separate crRNA and tracrRNA) or as one molecule (sgRNA), and optionally in the form of a complex with a Cas protein. The gRNA can also be provided in the form of DNA encoding the gRNA. The DNA encoding the gRNA can encode a single RNA molecule (sgRNA) or separate RNA molecules (e.g., separate crRNA and tracrRNA). In the latter case, the DNA encoding the gRNA can be provided as one DNA molecule or as separate DNA molecules encoding the crRNA and tracrRNA, respectively.

When a gRNA is provided in the form of DNA, the gRNA can be transiently, conditionally, or constitutively expressed in the cell. DNAs encoding gRNAs can be stably integrated into the genome of the cell and operably linked to a promoter active in the cell. Alternatively, DNAs encoding gRNAs can be operably linked to a promoter in an expression construct. For example, the DNA encoding the gRNA can be in a vector comprising a heterologous nucleic acid, such as a nucleic acid encoding a Cas protein. Alternatively, it can be in a vector or a plasmid that is separate from the vector comprising the nucleic acid encoding the Cas protein. Promoters that can be used in such expression constructs include promoters active, for example, in one or more of a eukaryotic cell, a human cell, a non-human cell, a mammalian cell, a non-human mammalian cell, a rodent cell, a mouse cell, a rat cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Such promoters can also be, for example, bidirectional promoters. Specific examples of suitable promoters include an RNA polymerase III promoter, such as a human U6 promoter, a rat U6 polymerase III promoter, or a mouse U6 polymerase III promoter.

Alternatively, gRNAs can be prepared by various other methods. For example, gRNAs can be prepared by in vitro transcription using, for example, T7 RNA polymerase (see, e.g., WO 2014/089290 and WO 2014/065596, each of which is herein incorporated by reference in its entirety for all purposes). Guide RNAs can also be a synthetically produced molecule prepared by chemical synthesis. For example, a guide RNA can be chemically synthesized to include 2′-O-methyl analogs and 3′ phosphorothioate internucleotide linkages at the first three 5′ and 3′ terminal RNA residues.

Guide RNAs (or nucleic acids encoding guide RNAs) can be in compositions comprising one or more guide RNAs (e.g., 1, 2, 3, 4, or more guide RNAs) and a carrier increasing the stability of the guide RNA (e.g., prolonging the period under given conditions of storage (e.g., -20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules. Such compositions can further comprise a Cas protein, such as a Cas9 protein, or a nucleic acid encoding a Cas protein.

As one example, a guide RNA targeting intron 1 of a human ALB gene can comprise, consist essentially of, or consist of the sequence set forth in any one of SEQ ID NOS: 62-125. Alternatively, a guide RNA targeting intron 1 of a human ALB gene can comprise, consist essentially of, or consist of a sequence that is at least 75%, at least 80%, at least 85%, at least 90%, or at least 95% identical to the DNA-targeting segment set forth in any one of SEQ ID NOS: 62-125. Alternatively, a guide RNA targeting intron 1 of a human ALB gene can comprise, consist essentially of, or consist of a sequence that is at least 90% or at least 95% identical to the DNA-targeting segment set forth in any one of SEQ ID NOS: 62-125. Alternatively, a guide RNA targeting intron 1 of a human ALB gene can comprise, consist essentially of, or consist of a sequence that differs by no more than 3, no more than 2, or no more than 1 nucleotide from the sequence set forth in any one of SEQ ID NOS: 62-125.

As another example, a guide RNA targeting intron 1 of a human ALB gene can comprise, consist essentially of, or consist of the sequence set forth in any one of SEQ ID NOS: 68, 100, 62, 94, 65, 97, 73, and 105. Alternatively, a guide RNA targeting intron 1 of a human ALB gene can comprise, consist essentially of, or consist of a sequence that is at least 75%, at least 80%, at least 85%, at least 90%, or at least 95% identical to the DNA-targeting segment set forth in any one of SEQ ID NOS: 68, 100, 62, 94, 65, 97, 73, and 105. Alternatively, a guide RNA targeting intron 1 of a human ALB gene can comprise, consist essentially of, or consist of a sequence that is at least 90% or at least 95% identical to the DNA-targeting segment set forth in any one of SEQ ID NOS: 68, 100, 62, 94, 65, 97, 73, and 105. Alternatively, a guide RNA targeting intron 1 of a human ALB gene can comprise, consist essentially of, or consist of a sequence that differs by no more than 3, no more than 2, or no more than 1 nucleotide from the sequence set forth in any one of SEQ ID NOS: 68, 100, 62, 94, 65, 97, 73, and 105.

As another example, a guide RNA targeting intron 1 of a human ALB gene can comprise, consist essentially of, or consist of the sequence set forth in SEQ ID NO: 68 or 100. Alternatively, a guide RNA targeting intron 1 of a human ALB gene can comprise, consist essentially of, or consist of a sequence that is at least 75%, at least 80%, at least 85%, at least 90%, or at least 95% identical to the DNA-targeting segment set forth in SEQ ID NO: 68 or 100. Alternatively, a guide RNA targeting intron 1 of a human ALB gene can comprise, consist essentially of, or consist of a sequence that is at least 90% or at least 95% identical to the DNA-targeting segment set forth in SEQ ID NO: 68 or 100. Alternatively, a guide RNA targeting intron 1 of a human ALB gene can comprise, consist essentially of, or consist of a sequence that differs by no more than 3, no more than 2, or no more than 1 nucleotide from the sequence set forth in SEQ ID NO: 68 or 100.

As another example, a guide RNA targeting intron 1 of a human ALB gene can comprise, consist essentially of, or consist of the sequence set forth in SEQ ID NO: 62 or 94. Alternatively, a guide RNA targeting intron 1 of a human ALB gene can comprise, consist essentially of, or consist of a sequence that is at least 75%, at least 80%, at least 85%, at least 90%, or at least 95% identical to the DNA-targeting segment set forth in SEQ ID NO: 62 or 94. Alternatively, a guide RNA targeting intron 1 of a human ALB gene can comprise, consist essentially of, or consist of a sequence that is at least 90% or at least 95% identical to the DNA-targeting segment set forth in SEQ ID NO: 62 or 94. Alternatively, a guide RNA targeting intron 1 of a human ALB gene can comprise, consist essentially of, or consist of a sequence that differs by no more than 3, no more than 2, or no more than 1 nucleotide from the sequence set forth in SEQ ID NO: 62 or 94.

As another example, a guide RNA targeting intron 1 of a human ALB gene can comprise, consist essentially of, or consist of the sequence set forth in SEQ ID NO: 65 or 97. Alternatively, a guide RNA targeting intron 1 of a human ALB gene can comprise, consist essentially of, or consist of a sequence that is at least 75%, at least 80%, at least 85%, at least 90%, or at least 95% identical to the DNA-targeting segment set forth in SEQ ID NO: 65 or 97. Alternatively, a guide RNA targeting intron 1 of a human ALB gene can comprise, consist essentially of, or consist of a sequence that is at least 90% or at least 95% identical to the DNA-targeting segment set forth in SEQ ID NO: 65 or 97. Alternatively, a guide RNA targeting intron 1 of a human ALB gene can comprise, consist essentially of, or consist of a sequence that differs by no more than 3, no more than 2, or no more than 1 nucleotide from the sequence set forth in SEQ ID NO: 65 or 97.

As another example, a guide RNA targeting intron 1 of a human ALB gene can comprise, consist essentially of, or consist of the sequence set forth in SEQ ID NO: 73 or 105. Alternatively, a guide RNA targeting intron 1 of a human ALB gene can comprise, consist essentially of, or consist of a sequence that is at least 75%, at least 80%, at least 85%, at least 90%, or at least 95% identical to the DNA-targeting segment set forth in SEQ ID NO: 73 or 105. Alternatively, a guide RNA targeting intron 1 of a human ALB gene can comprise, consist essentially of, or consist of a sequence that is at least 90% or at least 95% identical to the DNA-targeting segment set forth in SEQ ID NO: 73 or 105. Alternatively, a guide RNA targeting intron 1 of a human ALB gene can comprise, consist essentially of, or consist of a sequence that differs by no more than 3, no more than 2, or no more than 1 nucleotide from the sequence set forth in SEQ ID NO: 73 or 105.

### Guide RNA Target Sequences

Target DNAs for guide RNAs include nucleic acid sequences present in a DNA to which a DNA-targeting segment of a gRNA will bind, provided sufficient conditions for binding exist. Suitable DNA/RNA binding conditions include physiological conditions normally present in a cell. Other suitable DNA/RNA binding conditions (e.g., conditions in a cell-free system) are known in the art (see, e.g., Molecular Cloning: A Laboratory Manual, 3rd Ed. (Sambrook et al., Harbor Laboratory Press 2001), herein incorporated by reference in its entirety for all purposes). The strand of the target DNA that is complementary to and hybridizes with the gRNA can be called the “complementary strand,” and the strand of the target DNA that is complementary to the “complementary strand” (and is therefore not complementary to the Cas protein or gRNA) can be called “noncomplementary strand” or “template strand.”

The target DNA includes both the sequence on the complementary strand to which the guide RNA hybridizes and the corresponding sequence on the non-complementary strand (e.g., adj acent to the protospacer adjacent motif (PAM)). The term “guide RNA target sequence” as used herein refers specifically to the sequence on the non-complementary strand corresponding to (i.e., the reverse complement of) the sequence to which the guide RNA hybridizes on the complementary strand. That is, the guide RNA target sequence refers to the sequence on the non-complementary strand adjacent to the PAM (e.g., upstream or 5′ of the PAM in the case of Cas9). A guide RNA target sequence is equivalent to the DNA-targeting segment of a guide RNA, but with thymines instead of uracils. As one example, a guide RNA target sequence for an SpCas9 enzyme can refer to the sequence upstream of the 5′-NGG-3′ PAM on the non-complementary strand. A guide RNA is designed to have complementarity to the complementary strand of a target DNA, where hybridization between the DNA-targeting segment of the guide RNA and the complementary strand of the target DNA promotes the formation of a CRISPR complex. Full complementarity is not necessarily required, provided that there is sufficient complementarity to cause hybridization and promote formation of a CRISPR complex. If a guide RNA is referred to herein as targeting a guide RNA target sequence, what is meant is that the guide RNA hybridizes to the complementary strand sequence of the target DNA that is the reverse complement of the guide RNA target sequence on the non-complementary strand.

A target DNA or guide RNA target sequence can comprise any polynucleotide, and can be located, for example, in the nucleus or cytoplasm of a cell or within an organelle of a cell, such as a mitochondrion or chloroplast. A target DNA or guide RNA target sequence can be any nucleic acid sequence endogenous or exogenous to a cell. The guide RNA target sequence can be a sequence coding a gene product (e.g., a protein) or a non-coding sequence (e.g., a regulatory sequence) or can include both.

Site-specific binding and cleavage of a target DNA by a Cas protein can occur at locations determined by both (i) base-pairing complementarity between the guide RNA and the complementary strand of the target DNA and (ii) a short motif, called the protospacer adjacent motif (PAM), in the non-complementary strand of the target DNA. The PAM can flank the guide RNA target sequence. Optionally, the guide RNA target sequence can be flanked on the 3′ end by the PAM (e.g., for Cas9). Alternatively, the guide RNA target sequence can be flanked on the 5′ end by the PAM (e.g., for Cpf1). For example, the cleavage site of Cas proteins can be about 1 to about 10 or about 2 to about 5 base pairs (e.g., 3 base pairs) upstream or downstream of the PAM sequence (e.g., within the guide RNA target sequence). In the case of SpCas9, the PAM sequence (i.e., on the non-complementary strand) can be 5′-N₁GG-3′, where N₁ is any DNA nucleotide, and where the PAM is immediately 3′ of the guide RNA target sequence on the non-complementary strand of the target DNA. As such, the sequence corresponding to the PAM on the complementary strand (i.e., the reverse complement) would be 5′-CCN₂-3′, where N₂ is any DNA nucleotide and is immediately 5′ of the sequence to which the DNA-targeting segment of the guide RNA hybridizes on the complementary strand of the target DNA. In some such cases, N₁ and N₂ can be complementary and the N₁- N₂ base pair can be any base pair (e.g., N₁=C and N₂=G; N₁=G and N₂=C; N₁=A and N₂=T; or N₁=T, and N₂=A). In the case of Cas9 from S. aureus, the PAM can be NNGRRT or NNGRR, where N can A, G, C, or T, and R can be G or A. In the case of Cas9 from C. jejuni, the PAM can be, for example, NNNNACAC or NNNNRYAC, where N can be A, G, C, or T, and R can be G or A. In some cases (e.g., for FnCpf1), the PAM sequence can be upstream of the 5′ end and have the sequence 5′-TTN-3′. In the case of DpbCasX, the PAM can have the sequence 5′-TTCN-3′. In the case of CasΦ, the PAM can have the sequence 5′-TBN-3′, wherein B is G, T, or C.

An example of a guide RNA target sequence is a 20-nucleotide DNA sequence immediately preceding an NGG motif recognized by an SpCas9 protein. For example, two examples of guide RNA target sequences plus PAMs are GN₁₉NGG (SEQ ID NO: 5) or N₂₀NGG (SEQ ID NO: 6). See, e.g., WO 2014/165825, herein incorporated by reference in its entirety for all purposes. The guanine at the 5′ end can facilitate transcription by RNA polymerase in cells. Other examples of guide RNA target sequences plus PAMs can include two guanine nucleotides at the 5′ end (e.g., GGN₂₀NGG; SEQ ID NO: 7) to facilitate efficient transcription by T7 polymerase in vitro. See, e.g., WO 2014/065596, herein incorporated by reference in its entirety for all purposes. Other guide RNA target sequences plus PAMs can have between 4-22 nucleotides in length of SEQ ID NOS: 5-7, including the 5′ G or GG and the 3′ GG or NGG. Yet other guide RNA target sequences plus PAMs can have between 14 and 20 nucleotides in length of SEQ ID NOS: 5-7.

Formation of a CRISPR complex hybridized to a target DNA can result in cleavage of one or both strands of the target DNA within or near the region corresponding to the guide RNA target sequence (i.e., the guide RNA target sequence on the non-complementary strand of the target DNA and the reverse complement on the complementary strand to which the guide RNA hybridizes). For example, the cleavage site can be within the guide RNA target sequence (e.g., at a defined location relative to the PAM sequence). The “cleavage site” includes the position of a target DNA at which a Cas protein produces a single-strand break or a double-strand break. The cleavage site can be on only one strand (e.g., when a nickase is used) or on both strands of a double-stranded DNA. Cleavage sites can be at the same position on both strands (producing blunt ends; e.g. Cas9)) or can be at different sites on each strand (producing staggered ends (i.e., overhangs); e.g., Cpf1). Staggered ends can be produced, for example, by using two Cas proteins, each of which produces a single-strand break at a different cleavage site on a different strand, thereby producing a double-strand break. For example, a first nickase can create a single-strand break on the first strand of double-stranded DNA (dsDNA), and a second nickase can create a single-strand break on the second strand of dsDNA such that overhanging sequences are created. In some cases, the guide RNA target sequence or cleavage site of the nickase on the first strand is separated from the guide RNA target sequence or cleavage site of the nickase on the second strand by at least 2, 3, 4, 5, 6, 7, 8, 9, 10, 15, 20, 25, 30, 40, 50, 75, 100, 250, 500, or 1,000 base pairs.

The guide RNA target sequence can also be selected to minimize off-target modification or avoid off-target effects (e.g., by avoiding two or fewer mismatches to off-target genomic sequences).

As one example, a guide RNA targeting intron 1 of a human ALB gene can target the guide RNA target sequence set forth in any one of SEQ ID NOS: 126-157. As another example, a guide RNA targeting intron 1 of a human ALB gene can target at least 17, at least 18, at least 19, or at least 20 contiguous nucleotides of the guide RNA target sequence set forth in any one of SEQ ID NOS: 126-157.

As another example, a guide RNA targeting intron 1 of a human ALB gene can target the guide RNA target sequence set forth in any one of SEQ ID NOS: 132, 126, 129, and 137. As another example, a guide RNA targeting intron 1 of a human ALB gene can target at least 17, at least 18, at least 19, or at least 20 contiguous nucleotides of the guide RNA target sequence set forth in any one of SEQ ID NOS: 132, 126, 129, and 137.

As another example, a guide RNA targeting intron 1 of a human ALB gene can target the guide RNA target sequence set forth in SEQ ID NO: 132. As another example, a guide RNA targeting intron 1 of a human ALB gene can target at least 17, at least 18, at least 19, or at least 20 contiguous nucleotides of the guide RNA target sequence set forth in SEQ ID NO: 132.

As another example, a guide RNA targeting intron 1 of a human ALB gene can target the guide RNA target sequence set forth in SEQ ID NO: 126. As another example, a guide RNA targeting intron 1 of a human ALB gene can target at least 17, at least 18, at least 19, or at least 20 contiguous nucleotides of the guide RNA target sequence set forth in SEQ ID NO: 126.

As another example, a guide RNA targeting intron 1 of a human ALB gene can target the guide RNA target sequence set forth in SEQ ID NO: 129. As another example, a guide RNA targeting intron 1 of a human ALB gene can target at least 17, at least 18, at least 19, or at least 20 contiguous nucleotides of the guide RNA target sequence set forth in SEQ ID NO: 129.

As another example, a guide RNA targeting intron 1 of a human ALB gene can target the guide RNA target sequence set forth in SEQ ID NO: 137. As another example, a guide RNA targeting intron 1 of a human ALB gene can target at least 17, at least 18, at least 19, or at least 20 contiguous nucleotides of the guide RNA target sequence set forth in SEQ ID NO: 137.

TABLE 6

Human ALB Intron 1 Guide RNA Target Sequences.

| Guide RNA Target Sequence | SEQ ID NO: |
|---|---|
| GAGCAACCTCACTCTTGTCT | 126 |
| ATGCATTTGTTTCAAAATAT | 127 |
| TGCATTTGTTTCAAAATATT | 128 |
| ATTTATGAGATCAACAGCAC | 129 |
| GATCAACAGCACAGGTTTTG | 130 |
| TTAAATAAAGCATAGTGCAA | 131 |
| TAAAGCATAGTGCAATGGAT | 132 |
| TAGTGCAATGGATAGGTCTT | 133 |
| TACTAAAACTTTATTTTACT | 134 |
| AAAGTTGAACAATAGAAAAA | 135 |
| AATGCATAATCTAAGTCAAA | 136 |
| TAATAAAATTCAAACATCCT | 137 |
| GCATCTTTAAAGAATTATTT | 138 |
| TTTGGCATTTATTTCTAAAA | 139 |
| TGTATTTGTGAAGTCTTACA | 140 |
| TCCTAGGTAAAAAAAAAAAA | 141 |
| TAATTTTCTTTTGCGCACTA | 142 |
| TGACTGAAACTTCACAGAAT | 143 |
| GACTGAAACTTCACAGAATA | 144 |
| TTCATTTTAGTCTGTCTTCT | 145 |
| ATTATCTAAGTTTGAATATA | 146 |
| AATTTTTAAAATAGTATTCT | 147 |
| TGAATTATTCTTCTGTTTAA | 148 |
| ATCATCCTGAGTTTTTCTGT | 149 |
| TTACTAAAACTTTATTTTAC | 150 |
| ACCTTTTTTTTTTTTTACCT | 151 |
| AGTGCAATGGATAGGTCTTT | 152 |
| TGATTCCTACAGAAAAACTC | 153 |
| TGGGCAAGGGAAGAAAAAAA | 154 |
| CCTCACTCTTGTCTGGGCAA | 155 |
| ACCTCACTCTTGTCTGGGCA | 156 |
| TGAGCAACCTCACTCTTGTC | 157 |

TABLE 7

Mouse Alb Intron 1 Guide RNA Target Sequences.

| Guide RNA Target Sequence | SEQ ID NO: |
|---|---|
| CACTCTTGTCTGTGGAAACA | 165 |

### Lipid Nanoparticles Comprising Nuclease Agents

Lipid nanoparticles comprising the nuclease agents (e.g., CRISPR/Cas systems) are also provided. The lipid nanoparticles can alternatively or additionally comprise a nucleic acid construct encoding a polypeptide of interest (e.g., multidomain therapeutic protein) as disclosed herein. For example, the lipid nanoparticles can comprise a nuclease agent (e.g., CRISPR/Cas system), can comprise a nucleic acid construct encoding a polypeptide of interest (e.g., multidomain therapeutic protein), or can comprise both a nuclease agent (e.g., a CRISPR/Cas system) and a nucleic acid construct encoding a polypeptide of interest (e.g., multidomain therapeutic protein). Regarding CRISPR/Cas systems, the lipid nanoparticles can comprise the Cas protein in any form (e.g., protein, DNA, or mRNA) and/or can comprise the guide RNA(s) in any form (e.g., DNA or RNA). In one example, the lipid nanoparticles comprise the Cas protein in the form of mRNA (e.g., a modified RNA as described herein) and the guide RNA(s) in the form of RNA (e.g., a modified guide RNA as disclosed herein). As another example, the lipid nanoparticles can comprise the Cas protein in the form of protein and the guide RNA(s) in the form of RNA). In a specific example, the guide RNA and the Cas protein are each introduced in the form of RNA via LNP-mediated delivery in the same LNP. As discussed in more detail elsewhere herein, one or more of the RNAs can be modified. For example, guide RNAs can be modified to comprise one or more stabilizing end modifications at the 5′ end and/or the 3′ end. Such modifications can include, for example, one or more phosphorothioate linkages at the 5′ end and/or the 3′ end and/or one or more 2′-O-methyl modifications at the 5′ end and/or the 3′ end. As another example, Cas mRNA modifications can include substitution with pseudouridine (e.g., fully substituted with pseudouridine), 5′ caps, and polyadenylation. As another example, Cas mRNA modifications can include substitution with N1-methyl-pseudouridine (e.g., fully substituted with N1-methyl-pseudouridine), 5′ caps, and polyadenylation. Other modifications are also contemplated as disclosed elsewhere herein. Delivery through such methods can result in transient Cas expression and/or transient presence of the guide RNA, and the biodegradable lipids improve clearance, improve tolerability, and decrease immunogenicity. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1 and WO 2017/173054 A1, each of which is herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as distearoylphosphatidylcholine or 1,2-distearoyl-sn-glycero-3-phosphocholine (DSPC). In another example, the other components can comprise a helper lipid such as cholesterol, an optional neutral lipid such as DSPC, and a stealth lipid such as S010, S024, S027, S031, or S033.

The LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Rep. 22(9):2227-2235 and WO 2017/173054 A1, each of which is herein incorporated by reference in its entirety for all purposes. In certain LNPs, the cargo can include a guide RNA or a nucleic acid encoding a guide RNA. In certain LNPs, the cargo can include an mRNA encoding a Cas nuclease, such as Cas9, and a guide RNA or a nucleic acid encoding a guide RNA. In certain LNPs, the cargo can include a nucleic acid construct encoding a polypeptide of interest (e.g., multidomain therapeutic protein) as described elsewhere herein. In certain LNPs, the cargo can include an mRNA encoding a Cas nuclease, such as Cas9, a guide RNA or a nucleic acid encoding a guide RNA, and a nucleic acid construct encoding a polypeptide of interest (e.g., multidomain therapeutic protein). In some LNPs, the lipid component comprises an amine lipid such as a biodegradable, ionizable lipid. In some instances, the lipid component comprises biodegradable, ionizable lipid, cholesterol, DSPC, and PEG-DMG. For example, Cas9 mRNA and gRNA can be delivered to cells and animals utilizing lipid formulations comprising ionizable lipid ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z, 12Z)-octadeca-9,12-dienoate), cholesterol, DSPC, and PEG2k-DMG.

In some examples, the LNPs comprise cationic lipids. In some examples, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., WO 2019/067992, WO 2017/173054, WO 2015/095340, and WO 2014/136086, each of which is herein incorporated by reference in its entirety for all purposes. In some examples, the LNPs comprise molar ratios of a cationic lipid amine to RNA phosphate (N:P) of about 4.5, about 5.0, about 5.5, about 6.0, or about 6.5. In some examples, the terms cationic and ionizable in the context of LNP lipids are interchangeable (e.g., wherein ionizable lipids are cationic depending on the pH).

The lipid for encapsulation and endosomal escape can be a cationic lipid. The lipid can also be a biodegradable lipid, such as a biodegradable ionizable lipid. One example of a suitable lipid is Lipid A or LP01, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. See, e.g., Finn et al. (2018) Cell Rep. 22(9):2227-2235 and WO 2017/173054 A1, each of which is herein incorporated by reference in its entirety for all purposes. Another example of a suitable lipid is Lipid B, which is ((5-((dimethylamino)methyl)-1,3-phenylene)bis(oxy))bis(octane-8,1-diyl)bis(decanoate), also called ((5-((dimethylamino)methyl)-1,3-phenylene)bis(oxy))bis(octane-8,1-diyl)bis(decanoate). Another example of a suitable lipid is Lipid C, which is 2-((4-(((3-(dimethylamino)propoxy)carbonyl)oxy)hexadecanoyl)oxy)propane-1,3-diyl(9Z,9′Z,12Z,12′Z)-bis(octadeca-9,12-dienoate). Another example of a suitable lipid is Lipid D, which is 3-(((3-(dimethylamino)propoxy)carbonyl)oxy)-13-(octanoyloxy)tridecyl 3-octylundecanoate. Other suitable lipids include heptatriaconta-6,9,28,31-tetraen-19-yl 4-(dimethylamino)butanoate (also known as [(6Z,9Z,28Z,31Z)-heptatriaconta-6,9,28,31-tetraen-19-yl] 4-(dimethylamino)butanoate or Dlin-MC3-DMA (MC3))).

Some such lipids suitable for use in the LNPs described herein are biodegradable in vivo.

Such lipids may be ionizable depending upon the pH of the medium they are in. For example, in a slightly acidic medium, the lipids may be protonated and thus bear a positive charge. Conversely, in a slightly basic medium, such as, for example, blood where pH is approximately 7.35, the lipids may not be protonated and thus bear no charge. In some embodiments, the lipids may be protonated at a pH of at least about 9, 9.5, or 10. The ability of such a lipid to bear a charge is related to its intrinsic pKa. For example, the lipid may, independently, have a pKa in the range of from about 5.8 to about 6.2.

Neutral lipids function to stabilize and improve processing of the LNPs. Examples of suitable neutral lipids include a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine or 1,2-distearoyl-sn-glycero-3-phosphocholine (DSPC), phosphocholine (DOPC), dimyristoylphosphatidylcholine (DMPC), phosphatidylcholine (PLPC), 1,2-diarachidonoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine, 1-stearoyl-2-oleoyl-sn-glycero-3-phosphocholine (SOPC), and combinations thereof. For example, the neutral phospholipid may be selected from the group consisting of distearoylphosphatidylcholine (DSPC) and dimyristoyl phosphatidyl ethanolamine (DMPE).

Helper lipids include lipids that enhance transfection. The mechanism by which the helper lipid enhances transfection can include enhancing particle stability. In certain cases, the helper lipid can enhance membrane fusogenicity. Helper lipids include steroids, sterols, and alkyl resorcinols. Examples of suitable helper lipids suitable include cholesterol, 5-heptadecylresorcinol, and cholesterol hemisuccinate. In one example, the helper lipid may be cholesterol or cholesterol hemisuccinate.

Stealth lipids include lipids that alter the length of time the nanoparticles can exist in vivo. Stealth lipids may assist in the formulation process by, for example, reducing particle aggregation and controlling particle size. Stealth lipids may modulate pharmacokinetic properties of the LNP. Suitable stealth lipids include lipids having a hydrophilic head group linked to a lipid moiety.

The hydrophilic head group of stealth lipid can comprise, for example, a polymer moiety selected from polymers based on PEG (sometimes referred to as poly(ethylene oxide)), poly(oxazoline), poly(vinyl alcohol), poly(glycerol), poly(N- vinylpyrrolidone), polyaminoacids, and poly N-(2-hydroxypropyl)methacrylamide. The term PEG means any polyethylene glycol or other polyalkylene ether polymer. In certain LNP formulations, the PEG, is a PEG-2K, also termed PEG 2000, which has an average molecular weight of about 2,000 daltons. See, e.g., WO 2017/173054 A1, herein incorporated by reference in its entirety for all purposes.

The lipid moiety of the stealth lipid may be derived, for example, from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. The dialkylglycerol or dialkylglycamide group can further comprise one or more substituted alkyl groups.

As one example, the stealth lipid may be selected from PEG-dilauroylglycerol, PEG-dimyristoylglycerol (PEG-DMG), PEG-dipalmitoylglycerol, PEG-distearoylglycerol (PEG-DSPE), PEG-dilaurylglycamide, PEG- dimyristylglycamide, PEG-dipalmitoylglycamide, and PEG-distearoylglycamide, PEG- cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn- glycero-3-phosphoethanolamine-N-[methoxy(polyethylene glycol)-2000] (PEG2k- DMPE),or 1,2-dimyristoyl-rac-glycero-3-methylpolyoxyethylene glycol-2000 (PEG2k-DMG), 1,2- distearoyl-sn-glycero-3-phosphoethanolamine-N-[methoxy(polyethylene glycol)-2000] (PEG2k-DSPE), 1,2-distearoyl-sn-glycerol, methoxypoly ethylene glycol (PEG2k-DSG), poly(ethylene glycol)-2000-dimethacrylate (PEG2k-DMA), and 1,2- distearyloxypropyl-3-amine-N-[methoxy(polyethylene glycol)-2000] (PEG2k-DSA). In one particular example, the stealth lipid may be PEG2k-DMG.

In some embodiments, the PEG lipid includes a glycerol group. In some embodiments, the PEG lipid includes a dimyristoylglycerol (DMG) group. In some embodiments, the PEG lipid comprises PEG2k. In some embodiments, the PEG lipid is a PEG-DMG. In some embodiments, the PEG lipid is a PEG2k-DMG. In some embodiments, the PEG lipid is 1,2-dimyristoyl-rac-glycero-3-methoxypolyethylene glycol-2000. In some embodiments, the PEG2k-DMG is 1,2-dimyristoyl-rac-glycero-3-methoxypolyethylene glycol-2000.

The LNPs can comprise different respective molar ratios of the component lipids in the formulation. The mol-% of the CCD lipid may be, for example, from about 30 mol-% to about 60 mol-%. The mol-% of the helper lipid may be, for example, from about 30 mol-% to about 60 mol-%. The mol-% of the neutral lipid may be, for example, from about 1 mol-% to about 20 mol-%. The mol-% of the stealth lipid may be, for example, from about 1 mol-% to about 10 mol-%

The LNPs can have different ratios between the positively charged amine groups of the biodegradable lipid (N) and the negatively charged phosphate groups (P) of the nucleic acid to be encapsulated. This may be mathematically represented by the equation N/P. For example, the N/P ratio may be from about 0.5 to about 100. The N/P ratio can also be from about 4 to about 6.

In some LNPs, the cargo can comprise Cas mRNA (e.g., Cas9 mRNA) and gRNA. The Cas mRNA and gRNAs can be in different ratios. For example, the LNP formulation can include a ratio of Cas mRNA to gRNA nucleic acid ranging from about 25:1 to about 1:25. Alternatively, the LNP formulation can include a ratio of Cas mRNA to gRNA nucleic acid of from about 2:1 to about 1:2. In specific examples, the ratio of Cas mRNA to gRNA can be about 2:1.

In some LNPs, the cargo can comprise a nucleic acid construct encoding a polypeptide of interest (e.g., multidomain therapeutic protein) and gRNA. The nucleic acid construct encoding a polypeptide of interest (e.g., multidomain therapeutic protein) and gRNAs can be in different ratios. For example, the LNP formulation can include a ratio of nucleic acid construct to gRNA nucleic acid ranging from about 25:1 to about 1:25.

A specific example of a suitable LNP has a nitrogen-to-phosphate (N/P) ratio of about 4.5 and contains biodegradable cationic lipid, cholesterol, DSPC, and PEG2k-DMG in an about 45:44:9:2 molar ratio (about 45:about 44:about 9:about 2). The biodegradable cationic lipid can be (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. See, e.g., Finn et al. (2018) Cell Rep. 22(9):2227-2235, herein incorporated by reference in its entirety for all purposes. The Cas9 mRNA can be in an about 1:1 (about 1:about 1) ratio by weight to the guide RNA. Another specific example of a suitable LNP contains Dlin-MC3-DMA (MC3), cholesterol, DSPC, and PEG-DMG in an about 50:38.5:10:1.5 molar ratio (about 50:about 38.5:about 10:about 1.5). The Cas9 mRNA can be in an about 1:2 ratio (about 1:about 2)by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Another specific example of a suitable LNP has a nitrogen-to-phosphate (N/P) ratio of about 6 and contains biodegradable cationic lipid, cholesterol, DSPC, and PEG2k-DMG in an about 50:38:9:3 molar ratio (about 50:about 38:about 9:about 3). The biodegradable cationic lipid can be Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). The Cas9 mRNA can be in an about 1:2 ratio (about 1:about 2) by weight to the guide RNA. The Cas9 mRNA can be in an about 1:1 ratio (about 1:about 1)by weight to the guide RNA. The Cas9 mRNA can be in an about 2:1 (about 2:about 1) ratio by weight to the guide RNA.

Another specific example of a suitable LNP has a nitrogen-to-phosphate (N/P) ratio of about 3 and contains a cationic lipid, a structural lipid, cholesterol (e.g., cholesterol (ovine) (Avanti 700000)), and PEG2k-DMG (e.g., PEG-DMG 2000 (NOF America-SUNBRIGHT® GM-020(DMG-PEG)) in an about 50:10:38.5:1.5 ratio (about 50:about 10:about 38.5:about 1.5) or an about 47:10:42:1 ratio (about 47:about 10:about 42:about 1). The structural lipid can be, for example, DSPC (e.g., DSPC (Avanti 850365)), SOPC, DOPC, or DOPE. The cationic/ionizable lipid can be, for example, Dlin-MC3-DMA (e.g., Dlin-MC3-DMA (Biofine International)). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Another specific example of a suitable LNP contains Dlin-MC3-DMA, DSPC, cholesterol, and a PEG lipid in an about 45:9:44:2 ratio (about 45:about 9:about 44:about 2). Another specific example of a suitable LNP contains Dlin-MC3-DMA, DOPE, cholesterol, and PEG lipid or PEG DMG in an about 50:10:39:1 ratio (about 50:about 10:about 39:about 1). Another specific example of a suitable LNP has Dlin-MC3-DMA, DSPC, cholesterol, and PEG2k-DMG at an about 55:10:32.5:2.5 ratio (about 55:about 10:about 32.5:about 2.5). Another specific example of a suitable LNP has Dlin-MC3-DMA, DSPC, cholesterol, and PEG-DMG in an about 50:10:38.5:1.5 ratio (about 50:about 10:about 38.5:about 1.5). Another specific example of a suitable LNP has Dlin-MC3-DMA, DSPC, cholesterol, and PEG-DMG in an about 50:10:38.5:1.5 ratio (about 50:about 10:about 38.5:about 1.5). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Other examples of suitable LNPs can be found, e.g., in WO 2019/067992, WO 2020/082042, US 2020/0270617, WO 2020/082041, US 2020/0268906, WO 2020/082046 (see, e.g., pp. 85-86), and US 2020/0289628, each of which is herein incorporated by reference in its entirety for all purposes.

### Vectors Comprising Nuclease Agents

The nuclease agents disclosed herein (e.g., ZFN, TALEN, or CRISPR/Cas) can be provided in a vector for expression. A vector can comprise additional sequences such as, for example, replication origins, promoters, and genes encoding antibiotic resistance.

Some vectors may be circular. Alternatively, the vector may be linear. The vector can be in the packaged for delivered via a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

Introduction of nucleic acids can also be accomplished by virus-mediated delivery, such as AAV-mediated delivery or lentivirus-mediated delivery. The vectors can be, for example, viral vectors such as adeno-associated virus (AAV) vectors. The AAV may be any suitable serotype and may be a single-stranded AAV (ssAAV) or a self-complementary AAV (scAAV). Other exemplary viruses/viral vectors include retroviruses, lentiviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viral vector may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper virus to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging.

Exemplary viral titers (e.g., AAV titers) include about 10¹² to about 10¹⁶ vg/mL. Other exemplary viral titers (e.g., AAV titers) include about 10¹² to about 10¹⁶ vg/kg of body weight.

Adeno-associated viruses (AAVs) are endemic in multiple species including human and non-human primates (NHPs). At least 12 natural serotypes and hundreds of natural variants have been isolated and characterized to date. See, e.g., Li et al. (2020) Nat. Rev. Genet. 21:255-272, herein incorporated by reference in its entirety for all purposes. AAV particles are naturally composed of a non-enveloped icosahedral protein capsid containing a single-stranded DNA (ssDNA) genome. The DNA genome is flanked by two inverted terminal repeats (ITRs) which serve as the viral origins of replication and packaging signals. The rep gene encodes four proteins required for viral replication and packaging whilst the cap gene encodes the three structural capsid subunits which dictate the AAV serotype, and the Assembly Activating Protein (AAP) which promotes virion assembly in some serotypes.

Recombinant AAV (rAAV) is currently one of the most commonly used viral vectors used in gene therapy to treat human diseases by delivering therapeutic transgenes to target cells in vivo. Indeed, rAAV vectors are composed of icosahedral capsids similar to natural AAVs, but rAAV virions do not encapsidate AAV protein-coding or AAV replicating sequences. These viral vectors are non-replicating. The only viral sequences required in rAAV vectors are the two ITRs, which are needed to guide genome replication and packaging during manufacturing of the rAAV vector. rAAV genomes are devoid of AAV rep and cap genes, rendering them non-replicating in vivo. rAAV vectors are produced by expressing rep and cap genes along with additional viral helper proteins in trans, in combination with the intended transgene cassette flanked by AAV ITRs.

In therapeutic rAAV genomes, a gene expression cassette is placed between ITR sequences. Typically, rAAV genome cassettes comprise of a promoter to drive expression of a therapeutic transgene, followed by polyadenylation sequence. The ITRs flanking a rAAV expression cassette are usually derived from AAV2, the first serotype to be isolated and converted into a recombinant viral vector. Since then, most rAAV production methods rely on AAV2 Rep-based packaging systems. See, e.g., Colella et al. (2017) Mol. Ther. Methods Clin. Dev. 8:87-104, herein incorporated by reference in its entirety for all purposes.

Some non-limiting examples of ITRs that can be used include ITRs comprising, consisting essentially of, or consisting of SEQ ID NO: 158, SEQ ID NO: 159, or SEQ ID NO: 160. Other examples of ITRs comprise one or more mutations compared to SEQ ID NO: 158, SEQ ID NO: 159, or SEQ ID NO: 160 and can be at least 90%, at least 91%, at least 92%, at least 93%, at least 94%, at least 95%, at least 96%, at least 97%, at least 98%, or at least 99% identical to SEQ ID NO: 158, SEQ ID NO: 159, or SEQ ID NO: 160. In some rAAV genomes disclosed herein, the nucleic acid encoding the nuclease agent (or component thereof) is flanked on both sides by the same ITR (i.e., the ITR on the 5′ end, and the reverse complement of the ITR on the 3′ end, such as SEQ ID NO: 158 on the 5′ end and SEQ ID NO: 168 on the 3′ end, or SEQ ID NO: 159 on the 5′ end and SEQ ID NO: 710 on the 3′ end, or SEQ ID NO: 160 on the 5′ end and SEQ ID NO: 711 on the 3′ end). In one example, the ITR on each end can comprise, consist essentially of, or consist of SEQ ID NO: 158 (i.e., SEQ ID NO: 158 on the 5′ end, and the reverse complement on the 3′ end). In another example, the ITR on each end can comprise, consist essentially of, or consist of SEQ ID NO: 159 (i.e., SEQ ID NO: 159 on the 5′ end, and the reverse complement on the 3′ end). In one example, the ITR on at least one end comprises, consists essentially of, or consists of SEQ ID NO: 160. In one example, the ITR on the 5′ end comprises, consists essentially of, or consists of SEQ ID NO: 160. In one example, the ITR on the 3′ end comprises, consists essentially of, or consists of SEQ ID NO: 160. In one example, the ITR on each end can comprise, consist essentially of, or consist of SEQ ID NO: 160 (i.e., SEQ ID NO: 160 on the 5′ end, and the reverse complement on the 3′ end). In one example, the ITR on each end can comprise, consist essentially of, or consist of SEQ ID NO: 160. In other rAAV genomes disclosed herein, the nucleic acid encoding the nuclease agent (or component thereof) is flanked by different ITRs on each end. In one example, the ITR on one end comprises, consists essentially of, or consists of SEQ ID NO: 158, and the ITR on the other end comprises, consists essentially of, or consists of SEQ ID NO: 159. In another example, the ITR on one end comprises, consists essentially of, or consists of SEQ ID NO: 158, and the ITR on the other end comprises, consists essentially of, or consists of SEQ ID NO: 160. In one example, the ITR on one end comprises, consists essentially of, or consists of SEQ ID NO: 159, and the ITR on the other end comprises, consists essentially of, or consists of SEQ ID NO: 160.

The specific serotype of a recombinant AAV vector influences its in vivo tropism to specific tissues. AAV capsid proteins are responsible for mediating attachment and entry into target cells, followed by endosomal escape and trafficking to the nucleus. Thus, the choice of serotype when developing a rAAV vector will influence what cell types and tissues the vector is most likely to bind to and transduce when injected in vivo. Several serotypes of rAAVs, including rAAV8, are capable of transducing the liver when delivered systemically in mice, NHPs and humans. See, e.g., Li et al. (2020) Nat. Rev. Genet. 21:255-272, herein incorporated by reference in its entirety for all purposes.

Once in the nucleus, the ssDNA genome is released from the virion and a complementary DNA strand is synthesized to generate a double-stranded DNA (dsDNA) molecule. Double-stranded AAV genomes naturally circularize via their ITRs and become episomes which will persist extrachromosomally in the nucleus. Therefore, for episomal gene therapy programs, rAAV-delivered rAAV episomes provide long-term, promoter-driven gene expression in non-dividing cells. However, this rAAV-delivered episomal DNA is diluted out as cells divide. In contrast, the gene therapy described herein is based on gene insertion to allow long-term gene expression.

When specific rAAVs comprising specific sequences (e.g., specific bidirectional construct sequences or specific unidirectional construct sequences) are disclosed herein, they are meant to encompass the sequence disclosed or the reverse complement of the sequence. For example, if a bidirectional or unidirectional construct disclosed herein consists of the hypothetical sequence 5′-CTGGACCGA-3′, it is also meant to encompass the reverse complement of that sequence (5′-TCGGTCCAG-3′). Likewise, when rAAVs comprising bidirectional or unidirectional construct elements in a specific 5′ to 3′ order are disclosed herein, they are also meant to encompass the reverse complement of the order of those elements. For example, if an rAAV is disclosed herein that comprises a bidirectional construct that comprises from 5′ to 3′ a first splice acceptor, a first coding sequence, a first terminator, a reverse complement of a second terminator, a reverse complement of a second coding sequence, and a reverse complement of a second splice acceptor, it is also meant to encompass a construct comprising from 5′ to 3′ the second splice acceptor, the second coding sequence, the second terminator, a reverse complement of the first terminator, a reverse complement of the first coding sequence, and a reverse complement of the first splice acceptor. Single-stranded AAV genomes are packaged as either sense (plus-stranded) or anti-sense (minus-stranded genomes), and single-stranded AAV genomes of + and - polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

The ssDNA AAV genome consists of two open reading frames, Rep and Cap, flanked by two inverted terminal repeats that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2a, and VA) mediate AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Multiple serotypes of AAV have been identified. These serotypes differ in the types of cells they infect (i.e., their tropism), allowing preferential transduction of specific cell types. The term AAV includes, for example, AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. A “AAV vector” as used herein refers to an AAV vector comprising a heterologous sequence not of AAV origin (i.e., a nucleic acid sequence heterologous to AAV), typically comprising a sequence encoding an exogenous polypeptide of interest. The construct may comprise an AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV capsid sequence. In general, the heterologous nucleic acid sequence (the transgene) is flanked by at least one, and generally by two, AAV inverted terminal repeat sequences (ITRs). An AAV vector may either be single-stranded (ssAAV) or self-complementary (scAAV). Examples of serotypes for liver tissue include AAV3B, AAV5, AAV6, AAV7, AAV8, AAV9, AAVrh.74, and AAVhu.37, and particularly AAV8. In a specific example, the AAV vector comprising the nucleic acid construct can be recombinant AAV8 (rAAV8). A rAAV8 vector as described herein is one in which the capsid is from AAV8. For example, an AAV vector using ITRs from AAV2 and a capsid of AAV8 is considered herein to be a rAAV8 vector.

Tropism can be further refined through pseudotyping, which is the mixing of a capsid and a genome from different viral serotypes. For example AAV⅖ indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y731F, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG.

To accelerate transgene expression, self-complementary AAV (scAAV) variants can be used. Because AAV depends on the cell’s DNA replication machinery to synthesize the complementary strand of the AAV’s single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

To increase packaging capacity, longer transgenes may be split between two AAV transfer plasmids, the first with a 3′ splice donor and the second with a 5′ splice acceptor. Upon co-infection of a cell, these viruses form concatemers, are spliced together, and the full-length transgene can be expressed. Although this allows for longer transgene expression, expression is less efficient. Similar methods for increasing capacity utilize homologous recombination. For example, a transgene can be divided between two transfer plasmids but with substantial sequence overlap such that co-expression induces homologous recombination and expression of the full-length transgene.

In certain AAVs, the cargo can include nucleic acids encoding one or more guide RNAs (e.g., DNA encoding a guide RNA, or DNA encoding two or more guide RNAs). In certain AAVs, the cargo can include a nucleic acid (e.g., DNA) encoding a Cas nuclease, such as Cas9, and DNA encoding one or more guide RNAs (e.g., DNA encoding a guide RNA, or DNA encoding two or more guide RNAs). In certain AAVs, the cargo can include a nucleic acid construct encoding a polypeptide of interest (e.g., multidomain therapeutic protein). In certain AAVs, the cargo can include a nucleic acid (e.g., DNA) encoding a Cas nuclease, such as Cas9, a DNA encoding a guide RNA (or multiple guide RNAs), and a nucleic acid construct encoding a polypeptide of interest (e.g., multidomain therapeutic protein).

For example, Cas or Cas9 and one or more gRNAs (e.g., 1 gRNA or 2 gRNAs or 3 gRNAs or 4 gRNAs) can be delivered via LNP-mediated delivery (e.g., in the form of RNA) or adeno-associated virus (AAV)-mediated delivery (e.g., rAAV8-mediated delivery). For example, a Cas9 mRNA and a gRNA can be delivered via LNP-mediated delivery, or DNA encoding Cas9 and DNA encoding a gRNA can be delivered via AAV-mediated delivery. The Cas or Cas9 and the gRNA(s) can be delivered in a single AAV or via two separate AAVs. For example, a first AAV can carry a Cas or Cas9 expression cassette, and a second AAV can carry a gRNA expression cassette. Similarly, a first AAV can carry a Cas or Cas9 expression cassette, and a second AAV can carry two or more gRNA expression cassettes. Alternatively, a single AAV can carry a Cas or Cas9 expression cassette (e.g., Cas or Cas9 coding sequence operably linked to a promoter) and a gRNA expression cassette (e.g., gRNA coding sequence operably linked to a promoter). Similarly, a single AAV can carry a Cas or Cas9 expression cassette (e.g., Cas or Cas9 coding sequence operably linked to a promoter) and two or more gRNA expression cassettes (e.g., gRNA coding sequences operably linked to promoters). Different promoters can be used to drive expression of the gRNA, such as a U6 promoter or the small tRNA Gln. Likewise, different promoters can be used to drive Cas9 expression. For example, small promoters are used so that the Cas9 coding sequence can fit into an AAV construct. Similarly, small Cas9 proteins (e.g., SaCas9 or CjCas9 are used to maximize the AAV packaging capacity).

### C. Cells or Animals or Genomes

Cells or animals (i.e., subjects) comprising any of the above compositions (e.g., nucleic acid construct encoding a polypeptide of interest (e.g., multidomain therapeutic protein), nuclease agents, vectors, lipid nanoparticles, or any combination thereof) are also provided herein. Such cells or animals (or genomes) can be produced by the methods disclosed herein. For example, the cells or animals can comprise any of the nucleic acid constructs encoding a polypeptide of interest (e.g., multidomain therapeutic protein) described herein, any of the nuclease agents disclosed herein, or both. Such cells or animals (or genomes) can be neonatal cells or animals (or genomes). Alternatively, such cells or animals (or genomes) can be non-neonatal cells or animals (or genomes).

A neonatal subject (e.g., animal) can be a human subject up to or under the age of 1 year (52 weeks), preferably up to or under the age of 24 weeks, more preferably up to or under the age of 12 weeks, more preferably up to or under the age of 8 weeks, and even more preferably up to or under the age of 4 weeks. In certain embodiments, a neonatal human subject is up to 4 weeks of age. In certain embodiments, a neonatal human subject is up to 8 weeks of age. In another embodiment, a neonatal human subject is within 3 weeks after birth. In another embodiment, a neonatal human subject is within 2 weeks after birth. In another embodiment, a neonatal human subject is within 1 week after birth. In another embodiment, a neonatal human subject is within 7 days after birth. In another embodiment, a neonatal human subject is within 6 days after birth. In another embodiment, a neonatal human subject is within 5 days after birth. In another embodiment, a neonatal human subject is within 4 days after birth. In another embodiment, a neonatal human subject is within 3 days after birth. In another embodiment, a neonatal human subject is within 2 days after birth. In another embodiment, a neonatal human subject is within 1 day after birth. The time windows disclosed above are for human subjects and are also meant to cover the corresponding developmental time windows for other animals.

Neonatal cells can be cells of any neonatal subject. For example, they can be of a human subject up to or under the age of 1 year (52 weeks), preferably up to or under the age of 24 weeks, more preferably up to or under the age of 12 weeks, more preferably up to or under the age of 8 weeks, and even more preferably up to or under the age of 4 weeks. In certain embodiments, a neonatal human subject is up to 4 weeks of age. In certain embodiments, a neonatal human subject is up to 8 weeks of age. In another embodiment, a neonatal human subject is within 3 weeks after birth. In another embodiment, a neonatal human subject is within 2 weeks after birth. In another embodiment, a neonatal human subject is within 1 week after birth. In another embodiment, a neonatal human subject is within 7 days after birth. In another embodiment, a neonatal human subject is within 6 days after birth. In another embodiment, a neonatal human subject is within 5 days after birth. In another embodiment, a neonatal human subject is within 4 days after birth. In another embodiment, a neonatal human subject is within 3 days after birth. In another embodiment, a neonatal human subject is within 2 days after birth. In another embodiment, a neonatal human subject is within 1 day after birth. The time windows disclosed above are for human subjects and are also meant to cover the corresponding developmental time windows for other animals.

In some such cells or animals or genomes, the nucleic acid construct encoding a polypeptide of interest (e.g., multidomain therapeutic protein) can be genomically integrated at a target genomic locus, such as a safe harbor locus (e.g., an ALB locus or a human ALB locus, such as intron 1 of an ALB locus or a human ALB locus). In some such cells, animals, or genomes, the polypeptide of interest (e.g., multidomain therapeutic protein) encoded by the nucleic acid construct is expressed in the cell, animal, or genome. For example, if the nucleic acid construct encoding a polypeptide of interest (e.g., multidomain therapeutic protein) is integrated into an ALB locus (e.g., intron 1 of a human ALB locus), the polypeptide of interest (e.g., multidomain therapeutic protein) can be expressed from the ALB locus. The coding sequence for the polypeptide of interest (e.g., multidomain therapeutic protein) can be operably linked to an endogenous promoter at the target genomic locus upon integration into the target genomic locus, or it can be operably linked to an exogenous promoter present in the nucleic acid construct. If the nucleic acid construct is a bidirectional nucleic acid construct disclosed herein, the neonatal genome, neonatal cell, or neonatal animal can express the first polypeptide of interest or can express the second polypeptide of interest. In some neonatal genomes, neonatal cells, or neonatal animals, the target genomic locus is an ALB locus. For example, the nucleic acid construct can be genomically integrated in intron 1 of the endogenous ALB locus. Endogenous ALB exon 1 can then splice into the coding sequence for the polypeptide of interest (e.g., multidomain therapeutic protein) in the nucleic acid construct.

The target genomic locus at which the nucleic acid construct is stably integrated can be heterozygous for the nucleic acid construct encoding a polypeptide of interest (e.g., multidomain therapeutic protein) or homozygous for the nucleic acid construct encoding a polypeptide of interest (e.g., multidomain therapeutic protein). A diploid organism has two alleles at each genetic locus. Each pair of alleles represents the genotype of a specific genetic locus. Genotypes are described as homozygous if there are two identical alleles at a particular locus and as heterozygous if the two alleles differ.

The cells, neonatal, or genomes can be from any suitable species, such as eukaryotic cells or eukaryotes, or mammalian cells or mammals (e.g., non-human mammalian cells or non-human mammals, or human cells or humans). A mammal can be, for example, a non-human mammal, a human, a rodent, a rat, a mouse, or a hamster. Other non-human mammals include, for example, non-human primates, e.g., monkeys and apes. The term “non-human” excludes humans. Examples include, but are not limited to, human cells/humans, rodent cells/rodents, mouse cells/mice, rat cells/rats, and non-human primate cells/non-human primates. In a specific example, the cell is a human cell or the animal is a human. Likewise, cells can be any suitable type of cell. In a specific example, the cell is a liver cell such as a hepatocyte (e.g., a human liver cell or human hepatocyte).

The cells can be isolated cells (e.g., in vitro), ex vivo cells, or can be in vivo within an animal (i.e., in a subject). The cells can be mitotically competent cells or mitotically-inactive cells, meiotically competent cells or meiotically-inactive cells. Similarly, the cells can also be primary somatic cells or cells that are not a primary somatic cell. Somatic cells include any cell that is not a gamete, germ cell, gametocyte, or undifferentiated stem cell. For example, the neonatal cells can be liver cells, such as hepatocytes (e.g., mouse, non-human primate, or human hepatocytes).

The cells provided herein can be normal, healthy cells, or can be diseased or mutant-bearing cells. For example, the cells can have a deficiency of the polypeptide of interest or can be from a subject with deficiency of the polypeptide of interest. For example, the cells can have a GAA deficiency, can carry a mutation that results in a GAA deficiency, or can be from a subject with a GAA deficiency carrying a mutation that results in a GAA deficiency, or Pompe disease. In some embodiments, the cells are of a neonatal subject.

The cells provided herein can be dividing cells (e.g., actively dividing cells). Alternatively, the cells provided herein can be non-dividing cells.

### III. Therapeutic Methods and Methods for Introducing, Integrating, or Expressing a Nucleic Acid Encoding a Polypeptide of Interest in Cells or Subjects

The nucleic acid constructs and compositions disclosed herein can be used in methods of inserting or integrating a nucleic acid encoding a polypeptide of interest into a target genomic locus or methods of expressing a polypeptide of interest in a cell, in a population of cells, or in a subject (e.g., in a neonatal cell, in a population of neonatal cells, or in a neonatal subject).

The multidomain therapeutic protein nucleic acid constructs and compositions disclosed herein can be used in methods of introducing a nucleic acid construct encoding a multidomain therapeutic protein into a cell or a population of cells or a subject (e.g., in a cell or population of cells in a subject), methods of inserting or integrating a nucleic acid construct encoding a multidomain therapeutic protein into a target genomic locus in a cell or a population of cells or a subject (e.g., in a cell or population of cells in a subject), methods of expressing a multidomain therapeutic protein in a cell or a population of cells or a subject (e.g., in a cell or population of cells in a subject), methods of reducing glycogen accumulation in a cell or a population of cells or a tissue in a subject (e.g., in a cell or population of cells in a subject), methods of treating Pompe disease or GAA deficiency in a subject, and methods or preventing or reducing the onset of a sign or symptom of Pompe disease or GAA deficiency in a subject.

The multidomain therapeutic protein compositions disclosed herein (e.g., multidomain therapeutic protein nucleic acid constructs, or multidomain therapeutic protein nucleic acid constructs in combination with the nuclease agents (e.g., CRISPR/Cas systems)) are useful for the treatment of GAA deficiency or Pompe disease and/or ameliorating at least one symptom associated with GAA deficiency or Pompe disease (e.g., as compared to a control, untreated subject). The multidomain therapeutic protein compositions disclosed herein (e.g., multidomain therapeutic protein nucleic acid constructs, or multidomain therapeutic protein nucleic acid constructs in combination with the nuclease agents (e.g., CRISPR/Cas systems)) are also useful for preventing or reducing the onset of a sign or symptom of GAA deficiency or Pompe disease (e.g., as compared to a control, untreated subject). Likewise, the compositions disclosed herein can be used for the preparation of a pharmaceutical composition or medicament for treating a subject having GAA deficiency or Pompe disease.

With respect to GAA deficiency or Pompe disease, the terms “treat,” “treated,” “treating,” and “treatment,” include the administration of the multidomain therapeutic domain nucleic acid constructs disclosed herein (e.g., together with a nuclease agent disclosed herein) to subjects to prevent or delay the onset of the symptoms, complications, or biochemical indicia of GAA deficiency or Pompe disease, alleviating the symptoms or arresting or inhibiting further development of GAA deficiency or Pompe disease. Treatment may be prophylactic (to prevent or delay the onset of GAA deficiency or Pompe disease, or to prevent the manifestation of clinical or subclinical symptoms thereof) or therapeutic suppression or alleviation of symptoms after the manifestation of GAA deficiency or Pompe disease.

GAA deficiency refers expression and/or activity levels of GAA being lower in the subject (e.g., neonatal subject) than normal GAA expression and/or activity levels, such that the normal functions of GAA are not fully carried out in the subject (e.g., resulting in Pompe disease). Pompe disease is also known as acid maltase deficiency, acid maltase deficiency disease, alpha-1,4-glucosidase deficiency, AMD, deficiency of alpha-glucosidase, GAA deficiency, glycogen storage disease type II, glycogenosis type II, GSD II, GSD2, and Pompe’s disease.

Pompe disease is an inherited disorder caused by the buildup of glycogen in the body’s cells. The accumulation of glycogen in certain organs and tissues, especially muscles, impairs their ability to function normally. Different types of Pompe disease differ in severity and the age at which they appear. These types are known as infantile-onset Pompe disease (classic infantile-onset, and non-classic infantile-onset) and late-onset Pompe disease. Subjects with late-onset Pompe disease have higher GAA enzyme levels than are found in infantile-onset forms of the disease, but generally less than 40 percent of normal enzyme activity. Classic infantile-onset Pompe disease patients typically have less than 1 percent of GAA enzyme activity, while those with non-classic forms usually have less than 10 percent.

The classic form of infantile-onset Pompe disease begins within a few months of birth. Some phenotypes, such as cardiomyopathies, can be present at birth. Infants with this disorder typically experience muscle weakness (myopathy), poor muscle tone (hypotonia), an enlarged liver (hepatomegaly), and heart defects. Affected infants may also fail to gain weight and grow at the expected rate (failure to thrive) and have breathing problems. If untreated, this form of Pompe disease leads to death from heart failure in the first year of life.

The non-classic form of infantile-onset Pompe disease usually appears by age 1. It is characterized by delayed motor skills (such as rolling over and sitting) and progressive muscle weakness. The heart may be abnormally large (cardiomegaly), but affected individuals usually do not experience heart failure. The muscle weakness in this disorder leads to serious breathing problems, and most children with non-classic infantile-onset Pompe disease live only into early childhood.

The late-onset type of Pompe disease may not become apparent until later in childhood, adolescence, or adulthood. Late-onset Pompe disease is usually milder than the infantile-onset forms of this disorder and is less likely to involve the heart. Most individuals with late-onset Pompe disease experience progressive muscle weakness, especially in the legs and the trunk, including the muscles that control breathing. As the disorder progresses, breathing problems can lead to respiratory failure.

Mutations in the GAA gene can cause Pompe disease. The GAA gene encodes an enzyme called alpha-glucosidase. This enzyme is active in lysosomes. The enzyme normally breaks down glycogen into glucose. Mutations in the GAA gene prevent acid alpha-glucosidase from breaking down glycogen effectively, which allows this sugar to build up to toxic levels in lysosomes. This buildup damages organs and tissues throughout the body, particularly the muscles, leading to the progressive signs and symptoms of Pompe disease.

Since this is a genetic condition, the people who get this disease inherit it from a parent. It is common, however, that neither parent shows any symptoms. The disease is rare. In the United States, only 1 person in 40,000 is affected by Pompe disease. It can affect both males and females of all ethnic groups.

Symptoms of Pompe disease can be different, depending on when the disease makes itself present. For classic type, infant symptoms can include the following: weak muscles, poor muscle tone, enlarged liver, failure to gain weight and grow at the expected rate (failure to thrive), trouble breathing, feeding problems, infections in the respiratory system, and problems with hearing. For non-classic type, infant symptoms can include the following: motor skills delayed (such as rolling over and sitting), muscles get steadily weaker, abnormally large heart, and breathing problems. For late-onset type, symptoms can include the following: the legs and the trunk get steadily weaker, breathing problems, enlarged heart, increasing difficulty in walking, muscle pain over a large area, loss of the ability to exercise, falling often, frequent lung infections, shortness of breath when the person pushes himself or herself, headaches in the morning, becoming tired during the day, losing weight, cannot swallow as easily as before, irregular heartbeat, increased difficulty hearing, and higher levels of creatine kinase.

Pathology in Pompe disease can begin long before subjects present with symptoms. Pompe disease can be diagnosed by taking a blood sample, and enzymes in the blood are studied and counted. Confirmation can be made via DNA testing. For example, GAA enzyme activity can be measured by flow-injection tandem mass spectrometry, and full sequencing of the GAA gene is performed in newborns with low GAA enzyme activity. See, e.g., Ficicioglu et al. (2020) Int. J. Neonatal Screen. 6(4):89, Tang et al. (2020) Int. J. Neonatal Screen. 6(1):9, and Klug et al. (2020) Int. J. Neonatal Screen 6(1):11, each of which is herein incorporated by reference in its entirety for all purposes. GAA activity can be assessed by any known method. For example, to assess GAA activity (or deficiencies of activity), blood-based assays can measure GAA activity in dried blood spots or fresh blood. GAA activity can also be measured in fibroblasts from a skin biopsy or muscle biopsy. Other secondary measures can be measuring urine glucose tetrasaccharides by mass spectrometry. These can be combined with genetic analyses to diagnose in infantile and late onset Pompe disease. Asymptomatic subjects can be considered to have Pompe disease if diagnosed by genetic screening. For example, a subject described herein is considered to have Pompe disease, even if they are asymptomatic, if they have reduced GAA activity and a pathogenic GAA variant or mutation. Pathogenic GAA mutations and variants associated with Pompe disease are known. See, e.g., Ficicioglu et al. (2020) Int. J. Neonatal Screen. 6(4):89, Tang et al. (2020) Int. J. Neonatal Screen. 6(1):9, and Klug et al. (2020) Int. J. Neonatal Screen 6(1):11, each of which is herein incorporated by reference in its entirety for all purposes.

As is the case for several other lysosomal diseases, Pompe disease is currently treated by enzyme replacement therapy (ERT). Recombinant human GAA is delivered by intravenous infusion into patients every other week. While ERT has been successful in treating the cardiac manifestations of Pompe disease, skeletal muscle and the central nervous system (CNS) remain minimally treated by ERT.

The cells or populations of cells can be neonatal cells or populations of neonatal cells, and the subject can be neonatal subjects in some methods of introducing a nucleic acid construct encoding a multidomain therapeutic protein into a cell or a population of cells or a subject (e.g., in a cell or population of cells in a subject), methods of inserting or integrating a nucleic acid construct encoding a multidomain therapeutic protein into a target genomic locus in a cell or a population of cells or a subject (e.g., in a cell or population of cells in a subject), methods of expressing a multidomain therapeutic protein in a cell or a population of cells or a subject (e.g., in a cell or population of cells in a subject), methods of reducing glycogen accumulation in a cell or a population of cells or a tissue in a subject (e.g., in a cell or population of cells in a subject), and methods of treating Pompe disease or GAA deficiency in a subject. A neonatal subject can be a human subject up to or under the age of 1 year (52 weeks), preferably up to or under the age of 24 weeks, more preferably up to or under the age of 12 weeks, more preferably up to or under the age of 8 weeks, and even more preferably up to or under the age of 4 weeks. In certain embodiments, a neonatal human subject is up to 4 weeks of age. In certain embodiments, a neonatal human subject is up to 8 weeks of age. In another embodiment, a neonatal human subject is within 3 weeks after birth. In another embodiment, a neonatal human subject is within 2 weeks after birth. In another embodiment, a neonatal human subject is within 1 week after birth. In another embodiment, a neonatal human subject is within 7 days after birth. In another embodiment, a neonatal human subject is within 6 days after birth. In another embodiment, a neonatal human subject is within 5 days after birth. In another embodiment, a neonatal human subject is within 4 days after birth. In another embodiment, a neonatal human subject is within 3 days after birth. In another embodiment, a neonatal human subject is within 2 days after birth. In another embodiment, a neonatal human subject is within 1 day after birth. The time windows disclosed above are for human subjects and are also meant to cover the corresponding developmental time windows for other animals. As used herein, a “neonatal cell” is a cell of a neonatal subject, and a population of neonatal cells is a population of cells of a neonatal subject. In other methods, the cells or populations of cells are not neonatal cells and are not populations of neonatal cells, and the subjects are not neonatal subjects.

In one example, provided herein are methods of introducing a nucleic acid encoding a multidomain therapeutic protein into a cell or a population of cells or a subject in need thereof (e.g., in a cell or a population of cells in the subject). The cells or populations of cells can be neonatal cells or populations of neonatal cells, and the subject can be neonatal subjects in some methods. In other methods, the cells or populations of cells are not neonatal cells and are not populations of neonatal cells, and the subjects are not neonatal subjects. Such methods can comprise administering any of the multidomain therapeutic protein nucleic acid constructs described herein (or any of the compositions comprising a multidomain therapeutic protein nucleic acid construct described herein, including, for example, vectors or lipid nanoparticles) to the cell. The multidomain therapeutic protein nucleic acid construct can be administered together with a nuclease agent described herein, or can be administered alone. For example, the multidomain therapeutic protein nucleic acid construct can be one that expresses the multidomain therapeutic protein without being integrated into target genomic locus (e.g., an episomal vector or an expression vector in which the coding sequence for the multidomain therapeutic protein is operably linked to a promoter). In some methods, the multidomain therapeutic protein nucleic acid construct can be administered together with a nuclease agent described herein (e.g., simultaneously or sequentially in any order). The nuclease agent can cleave a nuclease target sequence within a target genomic locus (e.g., target gene), the multidomain therapeutic protein nucleic acid construct can be inserted into the target genomic locus to create a modified target genomic locus, and the multidomain therapeutic protein can be expressed from the modified target genomic locus. The multidomain therapeutic protein coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target sequence, the nucleic acid construct can be inserted into the ALB gene to create a modified ALB gene, and multidomain therapeutic protein can be expressed from the modified ALB gene.

In another example, provided herein are methods of expressing a multidomain therapeutic protein in a cell or a population of cells or a subject in need thereof (e.g., in a cell or a population of cells in the subject). The cells or populations of cells can be neonatal cells or populations of neonatal cells, and the subject can be neonatal subjects in some methods. In other methods, the cells or populations of cells are not neonatal cells and are not populations of neonatal cells, and the subjects are not neonatal subjects. Such methods can comprise administering any of the multidomain therapeutic protein nucleic acid constructs described herein (or any of the compositions comprising a multidomain therapeutic protein nucleic acid construct described herein, including, for example, vectors or lipid nanoparticles) to the cell. In some methods, the multidomain therapeutic protein nucleic acid construct or composition comprising the multidomain therapeutic protein nucleic acid construct can be administered without a nuclease agent (e.g., if the multidomain therapeutic protein nucleic acid construct comprises elements needed for expression of the multidomain therapeutic protein without integration into a target genomic locus). In some methods, the multidomain therapeutic protein nucleic acid construct can be administered together with a nuclease agent described herein (e.g., simultaneously or sequentially in any order). The nuclease agent can cleave a nuclease target sequence within a target genomic locus (e.g., target gene), the multidomain therapeutic protein nucleic acid construct can be inserted into the target genomic locus to create a modified target genomic locus, and multidomain therapeutic protein can be expressed from the modified target genomic locus. The multidomain therapeutic protein coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target sequence, the nucleic acid construct can be inserted into the ALB gene to create a modified ALB gene, and multidomain therapeutic protein can be expressed from the modified ALB gene.

In another example, provided herein are methods of inserting or integrating a multidomain therapeutic protein nucleic acid construct into a target genomic locus in a cell or a population of cells or a subject in need thereof (e.g., in a cell or a population of cells in the subject). The cells or populations of cells can be neonatal cells or populations of neonatal cells, and the subject can be neonatal subjects in some methods. In other methods, the cells or populations of cells are not neonatal cells and are not populations of neonatal cells, and the subjects are not neonatal subjects. Such methods can comprise administering any of the multidomain therapeutic protein nucleic acid constructs described herein (or any of the compositions comprising a multidomain therapeutic protein nucleic acid construct described herein, including, for example, vectors or lipid nanoparticles) to the cell. In some methods, the multidomain therapeutic protein nucleic acid construct or composition comprising the multidomain therapeutic protein nucleic acid construct can be administered together with a nuclease agent described herein (e.g., simultaneously or sequentially in any order). The nuclease agent can cleave a nuclease target sequence within a target genomic locus (e.g., target gene), the multidomain therapeutic protein nucleic acid construct can be inserted into the target genomic locus to create a modified target genomic locus, and the multidomain therapeutic protein can be expressed from the modified target genomic locus. The multidomain therapeutic protein coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target sequence, the nucleic acid construct can be inserted into the ALB gene to create a modified ALB gene, and multidomain therapeutic protein can be expressed from the modified ALB gene.

In any of the above methods, the cells can be from any suitable species, such as eukaryotic cells or mammalian cells (e.g., non-human mammalian cells or human cells). A mammal can be, for example, a non-human mammal, a human, a rodent, a rat, a mouse, or a hamster. Other non-human mammals include, for example, non-human primates, e.g., monkeys and apes. The term “non-human” excludes humans. Specific examples include, but are not limited to, human cells, rodent cells, mouse cells, rat cells, and non-human primate cells. In a specific example, the cell is a human cell. Likewise, cells can be any suitable type of cell. In a specific example, the cell is a liver cell such as a hepatocyte (e.g., a human liver cell or human hepatocyte). The cells can be neonatal cells, or they can be non-neonatal cells.

The cells can be isolated cells (e.g., in vitro), ex vivo cells, or can be in vivo within an animal (i.e., in a subject). In a specific example, the cell is in vivo (e.g., in a subject having a GAA deficiency or Pompe disease). The cells can be mitotically competent cells or mitotically-inactive cells, meiotically competent cells or meiotically-inactive cells. Similarly, the cells can also be primary somatic cells or cells that are not a primary somatic cell. Somatic cells include any cell that is not a gamete, germ cell, gametocyte, or undifferentiated stem cell. For example, the cells can be liver cells, such as hepatocytes (e.g., mouse, non-human primate, or human hepatocytes).

The cells provided herein can be normal, healthy cells, or can be diseased or mutant-bearing cells. For example, the cells can have a GAA deficiency or can be from a subject with GAA deficiency or Pompe disease.

Also provided are methods of treating a lysosomal alpha-glucosidase (GAA) deficiency in a subject in need thereof (e.g., a subject with Pompe disease). The Pompe disease can be any type of Pompe disease (e.g., infantile-onset Pompe disease (classic infantile-onset or non-classic infantile-onset) or late-onset Pompe disease). For example, the subject can have infantile-onset Pompe disease (e.g., classical infantile-onset Pompe disease). Pompe disease is described in more detail elsewhere herein. In some methods, the expressed multidomain therapeutic protein is delivered to and internalized by skeletal muscle, liver, or heart tissue in the subject. In some methods, the expressed multidomain therapeutic protein is delivered to and internalized by skeletal muscle or heart tissue in the subject. In some methods, the expressed multidomain therapeutic protein is delivered to and internalized by skeletal muscle tissue in the subject. In some methods, the expressed multidomain therapeutic protein is delivered to and internalized by liver tissue in the subject. In some methods, the expressed multidomain therapeutic protein is delivered to and internalized by heart tissue in the subject. In some methods, the expressed multidomain therapeutic protein is delivered to and internalized by skeletal muscle, liver, and heart tissue in the subject. In some methods, the expressed multidomain therapeutic protein is delivered to and internalized by skeletal muscle and heart tissue in the subject. In some methods, the expressed multidomain therapeutic protein is delivered to and internalized by skeletal muscle, liver, heart, or central nervous system tissue in the subject. In some methods, the expressed multidomain therapeutic protein is delivered to and internalized by skeletal muscle, heart, or central nervous system tissue in the subject. In some methods, the expressed multidomain therapeutic protein is delivered to and internalized by skeletal muscle tissue in the subject. In some methods, the expressed multidomain therapeutic protein is delivered to and internalized by liver tissue in the subject. In some methods, the expressed multidomain therapeutic protein is delivered to and internalized by heart tissue in the subject. In some methods, the expressed multidomain therapeutic protein is delivered to and internalized by central nervous system tissue in the subject. In some methods, the expressed multidomain therapeutic protein is delivered to and internalized by skeletal muscle, liver, heart, and central nervous system tissue in the subject. In some methods, the expressed multidomain therapeutic protein is delivered to and internalized by skeletal muscle, heart, and central nervous system tissue in the subject. In some methods, the method reduces glycogen accumulation in skeletal muscle, liver, or heart tissue in the subject. In some methods, the method reduces glycogen accumulation in skeletal muscle or heart tissue in the subject. In some methods, the method reduces glycogen accumulation in skeletal muscle tissue in the subject. In some methods, the method reduces glycogen accumulation in liver tissue in the subject. In some methods, the method reduces glycogen accumulation in heart tissue in the subject. For example, glycogen accumulation can be reduced in skeletal muscle, liver, and heart tissue in the subject. For example, glycogen accumulation can be reduced in skeletal muscle and heart tissue in the subject. In some methods, the method reduces glycogen accumulation in skeletal muscle, liver, heart, or central nervous system tissue in the subject. In some methods, the method reduces glycogen accumulation in skeletal muscle, heart, or central nervous system tissue in the subject. In some methods, the method reduces glycogen accumulation in skeletal muscle tissue in the subject. In some methods, the method reduces glycogen accumulation in liver tissue in the subject. In some methods, the method reduces glycogen accumulation in heart tissue in the subject. In some methods, the method reduces glycogen accumulation in central nervous system tissue in the subject. For example, glycogen accumulation can be reduced in skeletal muscle, liver, heart, and central nervous system tissue in the subject. For example, glycogen accumulation can be reduced in skeletal muscle, heart, and central nervous system tissue in the subject. In some cases, glycogen levels are reduced to wild type levels. In some cases, glycogen levels in skeletal muscle, heart, and/or central nervous system tissue are reduced to levels comparable to wild type levels at the same age. In some methods, the method improves muscle strength in the subject (e.g., restores muscle strength to wild type levels). In some methods, the method prevents loss of muscle strength in the subject compared to a control. In some methods, the method results in the subject having muscle strength comparable to wild type levels at the same age. Such methods can comprise administering any of the multidomain therapeutic protein nucleic acid constructs described herein (or any of the compositions comprising a multidomain therapeutic protein nucleic acid construct described herein, including, for example, vectors or lipid nanoparticles) to the subject such that a therapeutically effective level of multidomain therapeutic protein or GAA expression or a therapeutically effective level of circulating multidomain therapeutic protein or GAA is achieved in the subject. In some methods, the multidomain therapeutic protein nucleic acid construct or composition comprising the multidomain therapeutic protein nucleic acid construct can be administered without a nuclease agent (e.g., if the multidomain therapeutic protein nucleic acid construct comprises elements needed for expression of multidomain therapeutic protein without integration into a target genomic locus). In some methods, the multidomain therapeutic protein nucleic acid construct can be administered together with a nuclease agent described herein (e.g., simultaneously or sequentially in any order). The nuclease agent can cleave a nuclease target sequence within a target genomic locus (e.g., target gene), the multidomain therapeutic protein nucleic acid construct can be inserted into the target genomic locus to create a modified target genomic locus, and the multidomain therapeutic protein can be expressed from the modified target genomic locus (e.g., such that a therapeutically effective level of multidomain therapeutic protein or GAA expression or a therapeutically effective level of circulating multidomain therapeutic protein or GAA is achieved in the subject). The multidomain therapeutic protein coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target, the nucleic acid construct can be inserted into the ALB gene to create a modified ALB gene, and multidomain therapeutic protein can be expressed from the modified ALB gene (e.g., such that a therapeutically effective level of multidomain therapeutic protein or GAA expression or a therapeutically effective level of circulating multidomain therapeutic protein or GAA is achieved in the subject).

Also provided are methods of reducing glycogen accumulation in a cell or a population of cells or a tissue in a subject in need thereof (e.g., a subject with Pompe disease). Similarly, provided are methods of reducing glycogen accumulation in a cell or a population of cells. The Pompe disease can be any type of Pompe disease (e.g., infantile-onset Pompe disease (classic infantile-onset or non-classic infantile-onset) or late-onset Pompe disease). For example, the subject can have infantile-onset Pompe disease (e.g., classical infantile-onset Pompe disease). Pompe disease is described in more detail elsewhere herein. In some methods, the method reduces glycogen accumulation in skeletal muscle, liver, or heart tissue in the subject. In some methods, the method reduces glycogen accumulation in skeletal muscle or heart tissue in the subject. In some methods, the method reduces glycogen accumulation in skeletal muscle tissue in the subject. In some methods, the method reduces glycogen accumulation in liver tissue in the subject. In some methods, the method reduces glycogen accumulation in heart tissue in the subject. For example, glycogen accumulation can be reduced in skeletal muscle, liver, and heart tissue in the subject. For example, glycogen accumulation can be reduced in skeletal muscle and heart tissue in the subject. In some methods, the method reduces glycogen accumulation in skeletal muscle, liver, heart, or central nervous system tissue in the subject. In some methods, the method reduces glycogen accumulation in skeletal muscle, heart, or central nervous system tissue in the subject. In some methods, the method reduces glycogen accumulation in skeletal muscle tissue in the subject. In some methods, the method reduces glycogen accumulation in liver tissue in the subject. In some methods, the method reduces glycogen accumulation in heart tissue in the subject. In some methods, the method reduces glycogen accumulation in central nervous system tissue in the subject. For example, glycogen accumulation can be reduced in skeletal muscle, liver, heart, and central nervous system tissue in the subject. For example, glycogen accumulation can be reduced in skeletal muscle, heart, and central nervous system tissue in the subject. In some cases, glycogen levels are reduced to wild type levels. In some cases, glycogen levels in skeletal muscle, heart, and/or central nervous system tissue are reduced to levels comparable to wild type levels at the same age. In some methods, the method improves muscle strength in the subject (e.g., restores muscle strength to wild type levels). In some methods, the method prevents loss of muscle strength in the subject compared to a control. In some methods, the method results in the subject having muscle strength comparable to wild type levels at the same age. Such methods can comprise administering any of the multidomain therapeutic protein nucleic acid constructs described herein (or any of the compositions comprising a multidomain therapeutic protein nucleic acid construct described herein, including, for example, vectors or lipid nanoparticles) to the subject such that a therapeutically effective level of multidomain therapeutic protein or GAA expression or a therapeutically effective level of circulating multidomain therapeutic protein or GAA is achieved in the subject. In some methods, the multidomain therapeutic protein nucleic acid construct or composition comprising the multidomain therapeutic protein nucleic acid construct can be administered without a nuclease agent (e.g., if the multidomain therapeutic protein nucleic acid construct comprises elements needed for expression of multidomain therapeutic protein without integration into a target genomic locus). In some methods, the multidomain therapeutic protein nucleic acid construct can be administered together with a nuclease agent described herein (e.g., simultaneously or sequentially in any order). The nuclease agent can cleave a nuclease target sequence within a target genomic locus (e.g., target gene), the multidomain therapeutic protein nucleic acid construct can be inserted into the target genomic locus to create a modified target genomic locus, and the multidomain therapeutic protein can be expressed from the modified target genomic locus (e.g., such that a therapeutically effective level of multidomain therapeutic protein or GAA expression or a therapeutically effective level of circulating multidomain therapeutic protein or GAA is achieved in the subject). The multidomain therapeutic protein coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target, the nucleic acid construct can be inserted into the ALB gene to create a modified ALB gene, and multidomain therapeutic protein can be expressed from the modified ALB gene (e.g., such that a therapeutically effective level of multidomain therapeutic protein or GAA expression or a therapeutically effective level of circulating multidomain therapeutic protein or GAA is achieved in the subject).

Also provided are methods of treating Pompe disease in a subject. The Pompe disease can be any type of Pompe disease (e.g., infantile-onset Pompe disease (classic infantile-onset or non-classic infantile-onset) or late-onset Pompe disease). For example, the subject can have infantile-onset Pompe disease (e.g., classical infantile-onset Pompe disease). Pompe disease is described in more detail elsewhere herein. In some methods, the method reduces glycogen accumulation in skeletal muscle, liver, or heart tissue in the subject. In some methods, the method reduces glycogen accumulation in skeletal muscle or heart tissue in the subject. In some methods, the method reduces glycogen accumulation in skeletal muscle tissue in the subject. In some methods, the method reduces glycogen accumulation in liver tissue in the subject. In some methods, the method reduces glycogen accumulation in heart tissue in the subject. For example, glycogen accumulation can be reduced in skeletal muscle, liver, and heart tissue in the subject. For example, glycogen accumulation can be reduced in skeletal muscle and heart tissue in the subject. In some methods, the method reduces glycogen accumulation in skeletal muscle, liver, heart, or central nervous system tissue in the subject. In some methods, the method reduces glycogen accumulation in skeletal muscle, heart, or central nervous system tissue in the subject. In some methods, the method reduces glycogen accumulation in skeletal muscle tissue in the subject. In some methods, the method reduces glycogen accumulation in liver tissue in the subject. In some methods, the method reduces glycogen accumulation in heart tissue in the subject. In some methods, the method reduces glycogen accumulation in central nervous system tissue in the subject. For example, glycogen accumulation can be reduced in skeletal muscle, liver, heart, and central nervous system tissue in the subject. For example, glycogen accumulation can be reduced in skeletal muscle, heart, and central nervous system tissue in the subject. In some cases, glycogen levels are reduced to wild type levels. In some cases, glycogen levels in skeletal muscle, heart, and/or central nervous system tissue are reduced to levels comparable to wild type levels at the same age. In some methods, the method improves muscle strength in the subject (e.g., restores muscle strength to wild type levels). In some methods, the method prevents loss of muscle strength in the subject compared to a control. In some methods, the method results in the subject having muscle strength comparable to wild type levels at the same age. Such methods can comprise administering any of the multidomain therapeutic protein nucleic acid constructs described herein (or any of the compositions comprising a multidomain therapeutic protein nucleic acid construct described herein, including, for example, vectors or lipid nanoparticles) to the subject such that a therapeutically effective level of multidomain therapeutic protein or GAA expression or a therapeutically effective level of circulating multidomain therapeutic protein or GAA is achieved in the subject. In some methods, the multidomain therapeutic protein nucleic acid construct or composition comprising the multidomain therapeutic protein nucleic acid construct can be administered without a nuclease agent (e.g., if the multidomain therapeutic protein nucleic acid construct comprises elements needed for expression of multidomain therapeutic protein without integration into a target genomic locus). In some methods, the multidomain therapeutic protein nucleic acid construct can be administered together with a nuclease agent described herein (e.g., simultaneously or sequentially in any order). The nuclease agent can cleave a nuclease target sequence within a target genomic locus (e.g., target gene), the multidomain therapeutic protein nucleic acid construct can be inserted into the target genomic locus to create a modified target genomic locus, and the multidomain therapeutic protein can be expressed from the modified target genomic locus (e.g., such that a therapeutically effective level of multidomain therapeutic protein or GAA expression or a therapeutically effective level of circulating multidomain therapeutic protein or GAA is achieved in the subject). The multidomain therapeutic protein coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target, the nucleic acid construct can be inserted into the ALB gene to create a modified ALB gene, and multidomain therapeutic protein can be expressed from the modified ALB gene (e.g., such that a therapeutically effective level of multidomain therapeutic protein or GAA expression or a therapeutically effective level of circulating multidomain therapeutic protein or GAA is achieved in the subject).

Treatment refers to any administration or application of a therapeutic for disease or disorder in a subject, and includes inhibiting the disease, arresting its development, relieving one or more symptoms of the disease, curing the disease, or preventing reoccurrence of one or more symptoms of the disease. For example, treatment of Pompe disease may comprise alleviating symptoms of Pompe disease. Pompe disease is described in detail above and refers to a disorder caused by a missing or defective GAA gene or GAA polypeptide. The defective GAA gene or GAA polypeptide can result in reduced GAA expression and/or a activity of GAA.

Also provided are methods of preventing or reducing the onset of a sign or symptom of Pompe disease in a subject (e.g., as compared to an untreated, control subject). By preventing is meant the sign or symptom of the Pompe disease never becomes present. Such signs and symptoms are well-known and are described in more detail elsewhere herein. The Pompe disease can be any type of Pompe disease (e.g., infantile-onset Pompe disease (classic infantile-onset or non-classic infantile-onset) or late-onset Pompe disease). For example, Pompe disease can be infantile-onset Pompe disease (e.g., classical infantile-onset Pompe disease). Pompe disease is described in more detail elsewhere herein. In some methods, the method prevents or reduces glycogen accumulation in skeletal muscle, liver, or heart tissue in the subject. In some methods, the method prevents or reduces glycogen accumulation in skeletal muscle or heart tissue in the subject. In some methods, the method prevents or reduces glycogen accumulation in skeletal muscle tissue in the subject. In some methods, the method prevents or reduces glycogen accumulation in liver tissue in the subject. In some methods, the method prevents or reduces glycogen accumulation in heart tissue in the subject. For example, glycogen accumulation can be prevented or reduced in skeletal muscle, liver, and heart tissue in the subject. For example, glycogen accumulation can be prevented or reduced in skeletal muscle and heart tissue in the subject. In some methods, the method prevents or reduces glycogen accumulation in skeletal muscle, liver, heart, or central nervous system tissue in the subject. In some methods, the method prevents or reduces glycogen accumulation in skeletal muscle, heart, or central nervous system tissue in the subject. In some methods, the method prevents or reduces glycogen accumulation in skeletal muscle tissue in the subject. In some methods, the method prevents or reduces glycogen accumulation in liver tissue in the subject. In some methods, the method prevents or reduces glycogen accumulation in heart tissue in the subject. In some methods, the method prevents or reduces glycogen accumulation in central nervous system tissue in the subject. For example, glycogen accumulation can be prevented or reduced in skeletal muscle, liver, heart, and central nervous system tissue in the subject. For example, the onset of glycogen accumulation can be prevented or reduced in skeletal muscle, heart, and central nervous system tissue in the subject. Such methods can comprise administering any of the multidomain therapeutic protein nucleic acid constructs described herein (or any of the compositions comprising a multidomain therapeutic protein nucleic acid construct described herein, including, for example, vectors or lipid nanoparticles) to the subject such that a therapeutically effective level of multidomain therapeutic protein or GAA expression or a therapeutically effective level of circulating multidomain therapeutic protein or GAA is achieved in the subject. In some methods, the multidomain therapeutic protein nucleic acid construct or composition comprising the multidomain therapeutic protein nucleic acid construct can be administered without a nuclease agent (e.g., if the multidomain therapeutic protein nucleic acid construct comprises elements needed for expression of multidomain therapeutic protein without integration into a target genomic locus). In some methods, the multidomain therapeutic protein nucleic acid construct can be administered together with a nuclease agent described herein (e.g., simultaneously or sequentially in any order). The nuclease agent can cleave a nuclease target sequence within a target genomic locus (e.g., target gene), the multidomain therapeutic protein nucleic acid construct can be inserted into the target genomic locus to create a modified target genomic locus, and the multidomain therapeutic protein can be expressed from the modified target genomic locus (e.g., such that a therapeutically effective level of multidomain therapeutic protein or GAA expression or a therapeutically effective level of circulating multidomain therapeutic protein or GAA is achieved in the subject). The multidomain therapeutic protein coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target, the nucleic acid construct can be inserted into the ALB gene to create a modified ALB gene, and multidomain therapeutic protein can be expressed from the modified ALB gene (e.g., such that a therapeutically effective level of multidomain therapeutic protein or GAA expression or a therapeutically effective level of circulating multidomain therapeutic protein or GAA is achieved in the subject).

In some methods, a therapeutically effective amount of the multidomain therapeutic protein nucleic acid construct or the composition comprising the multidomain therapeutic protein nucleic acid construct or the combination of the multidomain therapeutic protein nucleic acid construct and the nuclease agent (e.g., CRISPR/Cas system) is administered to the subject. A therapeutically effective amount is an amount that produces the desired effect for which it is administered. The exact amount will depend on the purpose of the treatment, and will be ascertainable by one skilled in the art using known techniques. See, e.g., Lloyd (1999) The Art, Science and Technology of Pharmaceutical Compounding. In a specific example, serum levels of at least about 2 µg/mL or at least about 5 µg/mL of the multidomain therapeutic protein are considered therapeutically effective and correspond to complete correction of glycogen storage in muscles.

Therapeutic or pharmaceutical compositions comprising the compositions disclosed herein can be administered with suitable carriers, excipients, and other agents that are incorporated into formulations to provide improved transfer, delivery, tolerance, and the like. A multitude of appropriate formulations can be found in the formulary known to all pharmaceutical chemists: Remington’s Pharmaceutical Sciences, Mack Publishing Company, Easton, PA. See also Powell et al. “Compendium of excipients for parenteral formulations” PDA (1998) J. Pharm. Sci. Technol. 52:238-311. In certain embodiments, the pharmaceutical compositions are non-pyrogenic.

The compositions disclosed herein may be administered to relieve or prevent or decrease the severity of one or more of the symptoms of GAA deficiency or Pompe disease. Such symptoms are described in more detail elsewhere herein.

The subject in any of the above methods can be one in need of amelioration or treatment of GAA deficiency or Pompe disease. The subject in any of the above methods can be from any suitable species, such as a eukaryote or a mammal. A mammal can be, for example, a non-human mammal, a human, a rodent, a rat, a mouse, or a hamster. Other non-human mammals include, for example, non-human primates, e.g., monkeys and apes. The term “non-human” excludes humans. Specific examples of suitable species include, but are not limited to, humans, rodents, mice, rats, and non-human primates. In a specific example, the subject is a human. The subject in some methods can be a neonatal subject. In other methods, the subject is not a neonatal subject.

In methods in which a multidomain therapeutic protein nucleic acid construct is genomically integrated, any target genomic locus capable of expressing a gene can be used, such as a safe harbor locus (safe harbor gene) or an endogenous GAA locus. Such loci are described in more detail elsewhere herein. In a specific example, the target genomic locus can be an endogenous ALB locus, such as an endogenous human ALB locus. For example, the nucleic acid construct can be genomically integrated in intron 1 of the endogenous ALB locus. Endogenous ALB exon 1 can then splice into the coding sequence for the multidomain therapeutic protein in the nucleic acid construct.

Targeted insertion of the multidomain therapeutic protein nucleic acid construct comprising the multidomain therapeutic protein coding sequence into a target genomic locus, and particularly an endogenous ALB locus, offers multiple advantages. Such methods result in stable modification to allow for stable, long-term expression of the multidomain therapeutic protein coding sequence. With respect to the ALB locus, such methods are able to utilize the endogenous ALB promoter and regulatory regions to achieve therapeutically effective levels of expression. For example, the multidomain therapeutic protein coding sequence in the nucleic acid construct can comprise a promoterless gene, and the inserted nucleic acid construct can be operably linked to an endogenous promoter in the target genomic locus (e.g., ALB locus). Use of an endogenous promoter is advantageous because it obviates the need for inclusion of a promoter in the nucleic acid construct, allowing packaging of larger transgenes that may not normally package efficiently (e.g., in AAV). Alternatively, the multidomain therapeutic protein coding sequence in the nucleic acid construct can be operably linked to an exogenous promoter in the nucleic acid construct. Examples of types of promoters that can be used are disclosed elsewhere herein.

Optionally, some or all of the endogenous gene (e.g., endogenous ALB gene) at the target genomic locus can be expressed upon insertion of the multidomain therapeutic protein coding sequence from the nucleic acid construct. Alternatively, in some methods, none of the endogenous gene at the target genomic locus is expressed. As one example, the modified target genomic locus (e.g., modified ALB locus) after integration of the nucleic acid construct can encode a chimeric protein comprising an endogenous secretion signal (e.g., albumin secretion signal) and the multidomain therapeutic protein encoded by the nucleic acid construct. In another example, the first intron of an ALB locus can be targeted. The secretion signal peptide of ALB is encoded by exon 1 of the ALB gene. In such a scenario, a promoterless cassette bearing a splice acceptor and the multidomain therapeutic protein coding sequence will support expression and secretion of the multidomain therapeutic protein. Splicing between endogenous ALB exon 1 and the integrated multidomain therapeutic protein coding sequence creates a chimeric mRNA and protein including the endogenous ALB sequence encoded by exon 1 operably linked to the multidomain therapeutic protein sequence encoded by the integrated nucleic acid construct.

The multidomain therapeutic protein nucleic acid construct can be inserted into the target genomic locus by any means, including homologous recombination (HR) and non-homologous end joining (NHEJ) as described elsewhere herein. In a specific example, the multidomain therapeutic protein nucleic acid construct is inserted by NHEJ (e.g., does not comprise a homology arm and is inserted by NHEJ).

In another specific example, the nucleic acid construct can be inserted via homology-independent targeted integration (e.g., directional homology-independent targeted integration). For example, the multidomain therapeutic protein coding sequence in the nucleic acid construct can be flanked on each side by a target site for a nuclease agent (e.g., the same target site as in the target genomic locus, and the same nuclease agent being used to cleave the target site in the target genomic locus). The nuclease agent can then cleave the target sites flanking the multidomain therapeutic protein coding sequence. In a specific example, the nucleic acid construct is delivered AAV-mediated delivery, and cleavage of the target sites flanking the multidomain therapeutic protein coding sequence can remove the inverted terminal repeats (ITRs) of the AAV. Removal of the ITRs can make it easier to assess successful targeting, because presence of the ITRs can hamper sequencing efforts due to the repeated sequences. In some methods, the target site in the target genomic locus (e.g., a gRNA target sequence including the flanking protospacer adjacent motif) is no longer present if the multidomain therapeutic protein coding sequence is inserted into the target genomic locus in the correct orientation but it is reformed if the multidomain therapeutic protein coding sequence is inserted into the target genomic locus in the opposite orientation. This can help ensure that the multidomain therapeutic protein coding sequence is inserted in the correct orientation for expression.

In any of the above methods, the multidomain therapeutic protein nucleic acid construct can be administered simultaneously with the nuclease agent (e.g., CRISPR/Cas system) or not simultaneously (e.g., sequentially in any combination). For example, in a method comprising administering a composition comprising the multidomain therapeutic protein nucleic acid construct and a nuclease agent, they can be administered separately. For example, the multidomain therapeutic protein nucleic acid construct can be administered prior to the nuclease agent, subsequent to the nuclease agent, or at the same time as the nuclease agent. Any suitable methods of administering nucleic acid constructs and nuclease agents to cells can be used, particularly methods of administering to the liver, and examples of such methods are described in more detail elsewhere herein. In methods of treatment or in methods of targeting a cell in vivo in a subject, the nucleic acid construct can be inserted in particular types of cells in the subject. The method and vehicle for introducing the multidomain therapeutic protein nucleic acid construct and/or the nuclease agent into the subject can affect which types of cells in the subject are targeted. In some methods, for example, the nucleic acid construct is inserted into a target genomic locus (e.g., an endogenous ALB locus) in liver cells, such as hepatocytes. Methods and vehicles for introducing such constructs and nuclease agents into the subject (including methods and vehicles that target the liver or hepatocytes, such as lipid nanoparticle-mediated delivery and AAV-mediated delivery (e.g., rAAV8-mediated delivery) and intravenous injection), are disclosed in more detail elsewhere herein.

In methods in which a compositions comprising a nucleic acid construct (or vector or LNP) and a nuclease agent is administered (i.e., in methods in which a nucleic acid construct (or vector or LNP) and a nuclease agent are both administered), the nucleic acid construct and the nuclease agent can be administered simultaneously. Alternatively, the nucleic acid construct and the nuclease agent can be administered sequentially in any order. For example, the nucleic acid construct can be administered after the nuclease agent, or the nuclease agent can be administered after the nucleic acid construct. For example, the nuclease agent can be administered about 1 hour to about 48 hours, about 1 hour to about 24 hours, about 1 hour to about 12 hours, about 1 hour to about 6 hours, about 1 hour to about 2 hours, about 2 hours to about 48 hours, about 2 hours to about 24 hours, about 2 hours to about 12 hours, about 2 hours to about 6 hours, about 3 hours to about 48 hours, about 6 hours to about 48 hours, about 12 hours to about 48 hours, or about 24 hours to about 48 hours prior to or subsequent to administration of the nucleic acid construct.

In one example, the nucleic acid construct is administered about 4 hours, about 8 hours, about 12 hours, about 18 hours, about 1 day, about 2 days, about 3 days, about 4 days, about 5 days, about 6 days, or about 1 week prior to administering the nuclease agent. In another example, the nucleic acid construct is administered at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week prior to administering the nuclease agent. In another example, the nucleic acid construct is administered about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days prior to administering the nuclease agent.

In one example, the nucleic acid construct is administered about 4 hours, about 8 hours, about 12 hours, about 18 hours, about 1 day, about 2 days, about 3 days, about 4 days, about 5 days, about 6 days, or about 1 week after administering the nuclease agent. In another example, the nucleic acid construct is administered at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week after administering the nuclease agent. In another example, the nucleic acid construct is administered about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days after administering the nuclease agent.

In any of the above methods, the multidomain therapeutic protein nucleic acid construct and the nuclease agent (e.g., CRISPR/Cas system) can be administered using any suitable delivery system and known method. The nuclease agent components and multidomain therapeutic protein nucleic acid construct (e.g., the guide RNA, Cas protein, and multidomain therapeutic protein nucleic acid construct) can be delivered individually or together in any combination, using the same or different delivery methods as appropriate.

In methods in which a CRISPR/Cas system is used, a guide RNA can be introduced into or administered to a subject or cell, for example, in the form of an RNA (e.g., in vitro transcribed RNA, such as the modified guide RNAs disclosed herein) or in the form of a DNA encoding the guide RNA. When introduced in the form of a DNA, the DNA encoding a guide RNA can be operably linked to a promoter active in the cell or in a cell in the subject. For example, a guide RNA may be delivered via AAV and expressed in vivo under a U6 promoter. Such DNAs can be in one or more expression constructs. For example, such expression constructs can be components of a single nucleic acid molecule. Alternatively, they can be separated in any combination among two or more nucleic acid molecules (i.e., DNAs encoding one or more CRISPR RNAs and DNAs encoding one or more tracrRNAs can be components of a separate nucleic acid molecules).

Likewise, Cas proteins can be introduced into a subject or cell in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)), such as a modified mRNA as disclosed herein, or DNA). Optionally, the nucleic acid encoding the Cas protein can be codon optimized for efficient translation into protein in a particular cell or organism. For example, the nucleic acid encoding the Cas protein can be modified to substitute codons having a higher frequency of usage in a mammalian cell, a human cell, a rodent cell, a mouse cell, a rat cell, or any other host cell of interest, as compared to the naturally occurring polynucleotide sequence. When a nucleic acid encoding the Cas protein is introduced into a cell or a subject, the Cas protein can be transiently, conditionally, or constitutively expressed in the cell or in a cell in the subject.

In one example, the Cas protein is introduced in the form of an mRNA (e.g., a modified mRNA as disclosed herein), and the guide RNA is introduced in the form of RNA such as a modified gRNA as disclosed herein (e.g., together within the same lipid nanoparticle). Guide RNAs can be modified as disclosed elsewhere herein. Likewise, Cas mRNAs can be modified as disclosed elsewhere herein.

In methods in which a multidomain therapeutic protein nucleic acid construct is inserted following cleavage by a gene-editing system (e.g., a Cas protein), the gene-editing system (e.g., Cas protein) can cleave the target genomic locus to create a single-strand break (nick) or double-strand break, and the cleaved or nicked locus can be repaired by insertion of the multidomain therapeutic protein nucleic acid construct via non-homologous end joining (NHEJ)-mediated insertion or homology-directed repair. Optionally, repair with the multidomain therapeutic protein nucleic acid construct removes or disrupts the guide RNA target sequence(s) so that alleles that have been targeted cannot be re-targeted by the CRISPR/Cas reagents.

As explained in more detail elsewhere herein, the multidomain therapeutic protein nucleic acid constructs can comprise deoxyribonucleic acid (DNA) or ribonucleic acid (RNA), they can be single-stranded or double-stranded, and they can be in linear or circular form. The multidomain therapeutic protein nucleic acid constructs can be naked nucleic acids or can be delivered by viruses, such as AAV. In a specific example, the multidomain therapeutic protein nucleic acid construct can be delivered via AAV and can be capable of insertion into the target genomic locus (e.g., a safe harbor gene, an ALB gene, or intron 1 of an ALB gene) by non-homologous end joining (e.g., the multidomain therapeutic protein nucleic acid construct can be one that does not comprise a homology arm).

Some multidomain therapeutic protein nucleic acid constructs are capable of insertion by non-homologous end joining. In some cases, such multidomain therapeutic protein nucleic acid constructs do not comprise a homology arm. For example, such multidomain therapeutic protein nucleic acid constructs can be inserted into a blunt end double-strand break following cleavage with a Cas protein. In a specific example, the multidomain therapeutic protein nucleic acid construct can be delivered via AAV and can be capable of insertion by non-homologous end joining (e.g., the multidomain therapeutic protein nucleic acid construct can be one that does not comprise a homology arm).

In another example, the multidomain therapeutic protein nucleic acid construct can be inserted via homology-independent targeted integration. For example, the multidomain therapeutic protein nucleic acid construct can be flanked on each side by a guide RNA target sequence (e.g., the same target site as in the target genomic locus, and the CRISPR/Cas reagent (Cas protein and guide RNA) being used to cleave the target site in the target genomic locus). The Cas protein can then cleave the target sites flanking the nucleic acid insert. In a specific example, the multidomain therapeutic protein nucleic acid construct is delivered AAV-mediated delivery, and cleavage of the target sites flanking the nucleic acid insert can remove the inverted terminal repeats (ITRs) of the AAV. In some methods, the target site in the target genomic locus (e.g., a guide RNA target sequence including the flanking protospacer adjacent motif) is no longer present if the nucleic acid insert is inserted into the target genomic locus in the correct orientation but it is reformed if the nucleic acid insert is inserted into the target genomic locus in the opposite orientation.

The methods disclosed herein can comprise introducing or administering into an subject (e.g., an animal or mammal, such as a human) or cell a multidomain therapeutic protein nucleic acid construct and optionally a nuclease agent such as CRISPR/Cas reagents, including in the form of nucleic acids (e.g., DNA or RNA), proteins, or nucleic-acid-protein complexes. “Introducing” or “administering” includes presenting to the cell or subject the molecule(s) (e.g., nucleic acid(s) or protein(s)) in such a manner that it gains access to the interior of the cell or to the interior of cells within the subject. The introducing can be accomplished by any means, and two or more of the components (e.g., two of the components, or all of the components) can be introduced into the cell or subject simultaneously or sequentially in any combination. For example, a Cas protein can be introduced into a cell or subject before introduction of a guide RNA, or it can be introduced following introduction of the guide RNA. As another example, a multidomain therapeutic protein nucleic acid construct can be introduced prior to the introduction of a Cas protein and a guide RNA, or it can be introduced following introduction of the Cas protein and the guide RNA (e.g., the multidomain therapeutic protein nucleic acid construct can be administered about 1, 2, 3, 4, 8, 12, 24, 36, 48, or 72 hours before or after introduction of the Cas protein and the guide RNA). See, e.g., US 2015/0240263 and US 2015/0110762, each of which is herein incorporated by reference in its entirety for all purposes. In addition, two or more of the components can be introduced into the cell or subject by the same delivery method or different delivery methods. Similarly, two or more of the components can be introduced into a subject by the same route of administration or different routes of administration.

A guide RNA can be introduced into a subject or cell, for example, in the form of an RNA (e.g., in vitro transcribed RNA) or in the form of a DNA encoding the guide RNA. Guide RNAs can be modified as disclosed elsewhere herein. When introduced in the form of a DNA, the DNA encoding a guide RNA can be operably linked to a promoter active in the cell or in a cell in the subject. For example, a guide RNA may be delivered via AAV and expressed in vivo under a U6 promoter. Such DNAs can be in one or more expression constructs. For example, such expression constructs can be components of a single nucleic acid molecule. Alternatively, they can be separated in any combination among two or more nucleic acid molecules (i.e., DNAs encoding one or more CRISPR RNAs and DNAs encoding one or more tracrRNAs can be components of a separate nucleic acid molecules).

Likewise, Cas proteins can be provided in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)) or DNA. Cas RNAs can be modified as disclosed elsewhere herein. Optionally, the nucleic acid encoding the Cas protein can be codon optimized for efficient translation into protein in a particular cell or organism. For example, the nucleic acid encoding the Cas protein can be modified to substitute codons having a higher frequency of usage in a mammalian cell, a human cell, a rodent cell, a mouse cell, a rat cell, or any other host cell of interest, as compared to the naturally occurring polynucleotide sequence. When a nucleic acid encoding the Cas protein is introduced into a cell or a subject, the Cas protein can be transiently, conditionally, or constitutively expressed in the cell or in a cell in the subject.

Nucleic acids encoding Cas proteins or guide RNAs can be operably linked to a promoter in an expression construct. Expression constructs include any nucleic acid constructs capable of directing expression of a gene or other nucleic acid sequence of interest (e.g., a Cas gene) and which can transfer such a nucleic acid sequence of interest to a target cell. For example, the nucleic acid encoding the Cas protein can be in a vector comprising a DNA encoding one or more gRNAs. Alternatively, it can be in a vector or plasmid that is separate from the vector comprising the DNA encoding one or more gRNAs. Suitable promoters that can be used in an expression construct include promoters active, for example, in one or more of a eukaryotic cell, a human cell, a non-human cell, a mammalian cell, a non-human mammalian cell, a rodent cell, a mouse cell, a rat cell, a hamster cell, a pluripotent cell, an embryonic stem (ES) cell, an adult stem cell, a developmentally restricted progenitor cell, an induced pluripotent stem (iPS) cell, or a one-cell stage embryo. For example, a suitable promoter can be active in a liver cell such as a hepatocyte. Such promoters can be, for example, conditional promoters, inducible promoters, constitutive promoters, or tissue-specific promoters. Optionally, the promoter can be a bidirectional promoter driving expression of both a Cas protein in one direction and a guide RNA in the other direction. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes. Use of a bidirectional promoter to express genes encoding a Cas protein and a guide RNA simultaneously allows for the generation of compact expression cassettes to facilitate delivery. In preferred embodiments, promotors are accepted by regulatory authorities for use in humans. In certain embodiments, promotors drive expression in a liver cell.

Molecules (e.g., Cas proteins or guide RNAs or nucleic acids encoding) introduced into the subject or cell can be provided in compositions comprising a carrier increasing the stability of the introduced molecules (e.g., prolonging the period under given conditions of storage (e.g., -20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules.

Various methods and compositions are provided herein to allow for introduction of molecule (e.g., a nucleic acid or protein) into a cell or subject. Methods for introducing molecules into various cell types are known and include, for example, stable transfection methods, transient transfection methods, and virus-mediated methods.

Transfection protocols as well as protocols for introducing molecules into cells may vary. Non-limiting transfection methods include chemical-based transfection methods using liposomes; nanoparticles; calcium phosphate (Graham et al. (1973) Virology 52 (2): 456-67, Bacchetti et al. (1977) Proc. Natl. Acad. Sci. U.S.A. 74 (4):1590-4, and Kriegler, M (1991). Transfer and Expression: A Laboratory Manual. New York: W. H. Freeman and Company. pp. 96-97); dendrimers; or cationic polymers such as DEAE-dextran or polyethylenimine. Non-chemical methods include electroporation, sonoporation, and optical transfection. Particle-based transfection includes the use of a gene gun, or magnet-assisted transfection (Bertram (2006) Current Pharmaceutical Biotechnology 7, 277-28). Viral methods can also be used for transfection.

Introduction of nucleic acids or proteins into a cell can also be mediated by electroporation, by intracytoplasmic injection, by viral infection, by adenovirus, by adeno-associated virus, by lentivirus, by retrovirus, by transfection, by lipid-mediated transfection, or by nucleofection. Nucleofection is an improved electroporation technology that enables nucleic acid substrates to be delivered not only to the cytoplasm but also through the nuclear membrane and into the nucleus. In addition, use of nucleofection in the methods disclosed herein typically requires much fewer cells than regular electroporation (e.g., only about 2 million compared with 7 million by regular electroporation). In one example, nucleofection is performed using the LONZA® NUCLEOFECTOR™ system.

Introduction of molecules (e.g., nucleic acids or proteins) into a cell (e.g., a zygote) can also be accomplished by microinjection. In zygotes (i.e., one-cell stage embryos), microinjection can be into the maternal and/or paternal pronucleus or into the cytoplasm. If the microinjection is into only one pronucleus, the paternal pronucleus is preferable due to its larger size. Microinjection of an mRNA is preferably into the cytoplasm (e.g., to deliver mRNA directly to the translation machinery), while microinjection of a Cas protein or a polynucleotide encoding a Cas protein or encoding an RNA is preferable into the nucleus/pronucleus. Alternatively, microinjection can be carried out by injection into both the nucleus/pronucleus and the cytoplasm: a needle can first be introduced into the nucleus/pronucleus and a first amount can be injected, and while removing the needle from the one-cell stage embryo a second amount can be injected into the cytoplasm. If a Cas protein is injected into the cytoplasm, the Cas protein preferably comprises a nuclear localization signal to ensure delivery to the nucleus/pronucleus. Methods for carrying out microinjection are well known. See, e.g., Nagy et al. (Nagy A, Gertsenstein M, Vintersten K, Behringer R., 2003, Manipulating the Mouse Embryo. Cold Spring Harbor, New York: Cold Spring Harbor Laboratory Press); see also Meyer et al. (2010) Proc. Natl. Acad. Sci. U.S.A. 107:15022-15026 and Meyer et al. (2012) Proc. Natl. Acad. Sci. U.S.A. 109:9354-9359, each of which is herein incorporated by reference in its entirety for all purposes.

Other methods for introducing molecules (e.g., nucleic acid or proteins) into a cell or subject can include, for example, vector delivery, particle-mediated delivery, exosome-mediated delivery, lipid-nanoparticle-mediated delivery, cell-penetrating-peptide-mediated delivery, or implantable-device-mediated delivery. As specific examples, a nucleic acid or protein can be introduced into a cell or subject in a carrier such as a poly(lactic acid) (PLA) microsphere, a poly(D,L-lactic-coglycolic-acid) (PLGA) microsphere, a liposome, a micelle, an inverse micelle, a lipid cochleate, or a lipid microtubule. Some specific examples of delivery to a subject include hydrodynamic delivery, virus-mediated delivery (e.g., adeno-associated virus (AAV)-mediated delivery), and lipid-nanoparticle-mediated delivery.

Introduction of nucleic acids and proteins into cells or subjects can be accomplished by hydrodynamic delivery (HDD). For gene delivery to parenchymal cells, only essential DNA sequences need to be injected via a selected blood vessel, eliminating safety concerns associated with current viral and synthetic vectors. When injected into the bloodstream, DNA is capable of reaching cells in the different tissues accessible to the blood. Hydrodynamic delivery employs the force generated by the rapid injection of a large volume of solution into the incompressible blood in the circulation to overcome the physical barriers of endothelium and cell membranes that prevent large and membrane-impermeable compounds from entering parenchymal cells. In addition to the delivery of DNA, this method is useful for the efficient intracellular delivery of RNA, proteins, and other small compounds in vivo. See, e.g., Bonamassa et al. (2011) Pharm. Res. 28(4):694-701, herein incorporated by reference in its entirety for all purposes.

Introduction of nucleic acids can also be accomplished by virus-mediated delivery, such as AAV-mediated delivery or lentivirus-mediated delivery. Other exemplary viruses/viral vectors include retroviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression or longer-lasting expression. Viral vector may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper virus to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging.

Exemplary viral titers (e.g., AAV titers) include about 10¹² to about 10¹⁶ vg/mL. Other exemplary viral titers (e.g., AAV titers) include about 10¹² to about 10¹⁶ vg/kg of body weight.

Introduction of nucleic acids and proteins can also be accomplished by lipid nanoparticle (LNP)-mediated delivery. For example, LNP-mediated delivery can be used to deliver a combination of Cas mRNA and guide RNA or a combination of Cas protein and guide RNA. LNP-mediated delivery can be used to deliver a guide RNA in the form of RNA. In a specific example, the guide RNA and the Cas protein are each introduced in the form of RNA via LNP-mediated delivery in the same LNP. As discussed in more detail elsewhere herein, one or more of the RNAs can be modified. For example, guide RNAs can be modified to comprise one or more stabilizing end modifications at the 5′ end and/or the 3′ end. Such modifications can include, for example, one or more phosphorothioate linkages at the 5′ end and/or the 3′ end or one or more 2′-O-methyl modifications at the 5′ end and/or the 3′ end. As another example, Cas mRNA modifications can include substitution with pseudouridine (e.g., fully substituted with pseudouridine), 5′ caps, and polyadenylation. As another example, Cas mRNA modifications can include substitution with N1-methyl-pseudouridine (e.g., fully substituted with N1-methyl-pseudouridine), 5′ caps, and polyadenylation. Other modifications are also contemplated as disclosed elsewhere herein. Delivery through such methods can result in transient Cas expression and/or transient presence of the guide RNA, and the biodegradable lipids improve clearance, improve tolerability, and decrease immunogenicity. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1 and WO2017/173054 A1, each of which is herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as DSPC. In another example, the other components can comprise a helper lipid such as cholesterol, an optional neutral lipid such as DSPC, and a stealth lipid such as S010, S024, S027, S031, or S033.

The LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Rep. 22(9):2227-2235 and WO 2017/173054 A1, each of which is herein incorporated by reference in its entirety for all purposes. In certain LNPs, the cargo can include a guide RNA or a nucleic acid encoding a guide RNA. In certain LNPs, the cargo can include an mRNA encoding a Cas nuclease, such as Cas9, and a guide RNA or a nucleic acid encoding a guide RNA. In certain LNPs, the cargo can include a multidomain therapeutic protein nucleic acid construct. In certain LNPs, the cargo can include an mRNA encoding a Cas nuclease, such as Cas9, a guide RNA or a nucleic acid encoding a guide RNA, and a multidomain therapeutic protein nucleic acid construct. LNPs for use in the methods are described in more detail elsewhere herein.

The mode of delivery can be selected to decrease immunogenicity. For example, a Cas protein and a gRNA may be delivered by different modes (e.g., bi-modal delivery). These different modes may confer different pharmacodynamics or pharmacokinetic properties on the subject delivered molecule (e.g., Cas or nucleic acid encoding, gRNA or nucleic acid encoding, or multidomain therapeutic protein nucleic acid construct). For example, the different modes can result in different tissue distribution, different half-life, or different temporal distribution. Some modes of delivery (e.g., delivery of a nucleic acid vector that persists in a cell by autonomous replication or genomic integration) result in more persistent expression and presence of the molecule, whereas other modes of delivery are transient and less persistent (e.g., delivery of an RNA or a protein). Delivery of Cas proteins in a more transient manner, for example as mRNA or protein, can ensure that the Cas/gRNA complex is only present and active for a short period of time and can reduce immunogenicity caused by peptides from the bacterially-derived Cas enzyme being displayed on the surface of the cell by MHC molecules. Such transient delivery can also reduce the possibility of off-target modifications.

Administration in vivo can be by any suitable route including, for example, systemic routes of administration such as parenteral administration, e.g., intravenous, subcutaneous, intraarterial, or intramuscular. In a specific example, administration in vivo is intravenous.

Compositions comprising the guide RNAs and/or Cas proteins (or nucleic acids encoding the guide RNAs and/or Cas proteins) can be formulated using one or more physiologically and pharmaceutically acceptable carriers, diluents, excipients or auxiliaries. The formulation can depend on the route of administration chosen. Pharmaceutically acceptable means that the carrier, diluent, excipient, or auxiliary is compatible with the other ingredients of the formulation and not substantially deleterious to the recipient thereof. In a specific example, the route of administration and/or formulation or chosen for delivery to the liver (e.g., hepatocytes).

The methods disclosed herein can increase multidomain therapeutic protein or GAA protein levels and/or multidomain therapeutic protein or GAA activity levels in a cell or subject (e.g., circulating, serum, or plasma levels in a subject) and can comprise measuring multidomain therapeutic protein or GAA protein levels and/or multidomain therapeutic protein or GAA activity levels in a cell or subject (e.g., circulating, serum, or plasma levels in a subject). In one example, the methods result in increased expression of the multidomain therapeutic protein in the subject compared to a method comprising administering an episomal expression vector encoding the multidomain therapeutic protein. For example, the methods can result in increased serum levels of the multidomain therapeutic protein in the subject compared to a method comprising administering an episomal expression vector encoding the multidomain therapeutic protein. The methods can also result in increased multidomain therapeutic protein activity or GAA activity in the subject compared to a method comprising administering an episomal expression vector encoding the multidomain therapeutic protein. Levels of circulating multidomain therapeutic protein or GAA activity can be measured by using well-known methods.

In some methods, GAA activity and/or expression levels in a subject are increased to about or at least about 2%, about or at least about 10%, about or at least about 25%, about or at least about 40%, about or at least about 50%, about or at least about 75%, or at least about 100%, or more, of normal level. In some methods, GAA activity and/or expression levels in a subject are increased to about or at least about 40%, about or at least about 50%, about or at least about 75%, or at least about 100%, or more, of normal level. In certain embodiments, the level of expression or activity is measured in a cell or tissue in which a sign or symptom of the GAA loss of function is present. For example, when the loss of function results in muscle dysfunction, the level or activity of the multidomain therapeutic protein or GAA is measured in a muscle cell. It is understood that depending on the exogenous protein, the level of activity of the multidomain therapeutic protein may not compare 1:1 with a native GAA protein based on weight. In such embodiment, the relative activity of the multidomain therapeutic protein and the native GAA can be compared. In certain embodiments, the loss of function is nearly complete such that a relative activity cannot be determined. In certain embodiments, the comparison is made to an appropriate control subject. Selection of an appropriate control subject is within the ability of those of skill in the art. In certain embodiments, the level of expression is sufficient to treat at least one sign or symptom resulting from the loss of function of the GAA. GAA activity can be assessed by any known method. For example, to assess GAA activity (or deficiencies of activity), blood-based assays can measure GAA activity in dried blood spots or fresh blood. GAA activity can also be measured in fibroblasts from a skin biopsy or muscle biopsy. Other secondary measures can be measuring urine glucose tetrasaccharides by mass spectrometry.

In some methods, circulating multidomain therapeutic protein levels (i.e., serum levels) are about or at least about 0.5, about or at least about 1, about or at least about 2, about or at least about 3, about or at least about 4, about or at least about 5, about or at least about 6, about or at least about 7, about or at least about 8, about or at least about 9, or about or at least about 10 µg/mL. In some methods, multidomain therapeutic protein levels are at least about 1 µg/mL or about 1 µg/mL. In some methods, multidomain therapeutic protein levels are at least about 2 µg/mL or about 2 µg/mL. In some methods, multidomain therapeutic protein levels are at least about 5 µg/mL or about 5 µg/mL. In some methods, multidomain therapeutic protein levels are about 1 µg/mL to about 30 µg/mL, about 2 µg/mL to about 30 µg/mL, about 3 µg/mL to about 30 µg/mL, about 4 µg/mL to about 30 µg/mL, about 5 µg/mL to about 30 µg/mL, about 1 µg/mL to about 20 µg/mL, about 2 µg/mL to about 20 µg/mL, about 3 µg/mL to about 20 µg/mL, about 4 µg/mL to about 20 µg/mL, about 5 µg/mL to about 20 µg/mL. For example, the method can result in multidomain therapeutic protein levels of about 2 µg/mL to about 30 µg/mL or 2 µg/mL to about 20 µg/mL. For example, the method can result in multidomain therapeutic protein levels of about 5 µg/mL to about 30 µg/mL or 5 µg/mL to about 20 µg/mL. In some embodiments, the recited expression levels are at least 1 month after administration. In some embodiments, the recited expression levels are at least 2 months after administration. In some embodiments, the recited expression levels are at least 3 months after administration. In some embodiments, the recited expression levels are at least 4 months after administration. In some embodiments, the recited expression levels are at least 5 months after administration. In some embodiments, the recited expression levels are at least 6 months after administration. In some embodiments, the recited expression levels are at least 9 months after administration. In some embodiments, the recited expression levels are at least 12 months after administration.

In some methods, the method increases expression and/or activity of GAA or the multidomain therapeutic protein over the subject’s baseline expression and/or activity (i.e., expression and/or activity prior to administration). In some methods, the method increases expression and/or activity of GAA over the subject’s baseline expression and/or activity (i.e., expression and/or activity prior to administration. In some methods, GAA activity and/or GAA expression or serum levels in a subject are increased by about or at least about 10%, about or at least about 25%, about or at least about 50%, about or at least about 75%, or about or at least about 100%, or more, as compared to the subject’s GAA expression or serum levels and/or activity (e.g., GAA activity) before administration (i.e., the subject’s baseline levels). It is understood that depending on the multidomain therapeutic protein, the level of activity of the multidomain therapeutic protein may not compare 1:1 with a native protein based on weight. In such embodiment, the relative activity of the multidomain therapeutic protein and the native GAA can be compared. In certain embodiments, the loss of function is nearly complete such that a relative activity cannot be determined. In certain embodiments, the level of expression is sufficient to treat at least one sign or symptom resulting from the loss of function of the GAA.

In some methods, the method increases expression and/or activity of the multidomain therapeutic protein over the cell’s baseline expression and/or activity (i.e., expression and/or activity prior to administration). In some methods, the method increases expression and/or activity of GAA over the cell’s baseline expression and/or activity (i.e., expression and/or activity prior to administration. In some methods, GAA activity and/or expression levels in a cell or population of cells (e.g., liver cells, or hepatocytes) are increased by about or at least about 10%, about or at least about 25%, about or at least about 50%, about or at least about 75%, about or at least about 100%, or more, as compared to the GAA activity and/or expression levels before administration (i.e., the subject’s baseline levels). It is understood that depending on the multidomain therapeutic protein, the level of activity of the multidomain therapeutic protein may not compare 1:1 with a native GAA protein based on weight. In such embodiment, the relative activity of the multidomain therapeutic protein and the native GAA protein can be compared. In certain embodiments, the GAA loss of function is nearly complete such that a relative activity cannot be determined. In certain embodiments, the level of expression is sufficient to treat at least one sign or symptom resulting from the loss of function of the GAA.

In a specific example, the GAA activity levels in a subject are increased to no more than about 300%, no more than about 250%, no more than about 200%, or no more than about 150% of normal GAA activity levels.

In a specific example, the GAA activity levels in the subject are increased to at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels. In a specific example, the GAA activity levels in the subject are increased to at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels.

In a specific example, a subject has infantile-onset Pompe disease (e.g., classic infantile-onset Pompe disease), and the GAA activity levels in the subject are increased to at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels. In a specific example, a subject has infantile-onset Pompe disease (e.g., classic infantile-onset Pompe disease), and the GAA activity levels in the subject are increased to at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels.

In a specific example, a subject has infantile-onset Pompe disease (e.g., classic or non-classic infantile-onset Pompe disease), and the GAA activity levels in the subject are increased to at least about 2% at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels (e.g., at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels). In a specific example, a subject has infantile-onset Pompe disease (e.g., classic or non-classic infantile-onset Pompe disease), and the GAA activity levels in the subject are increased to at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels.

In a specific example, a subject has late-onset Pompe disease, and the GAA activity levels in the subject are increased to at least about 2% at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels (e.g., at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels).

In a specific example, a subject has infantile-onset Pompe disease (e.g., classic infantile-onset Pompe disease), and the GAA activity levels in the subject are increased to more than about 1%, more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal GAA activity levels. In a specific example, a subject has infantile-onset Pompe disease (e.g., classic infantile-onset Pompe disease), and the GAA activity levels in the subject are increased to more than about 40%, more than about 45%, more than about 50%, more than about 55%, more than about 60%, more than about 65%, more than about 70%, more than about 75%, more than about 80%, more than about 85%, more than about 90%, or more than about 100% of normal GAA activity levels

In a specific example, a subject has infantile-onset Pompe disease (e.g., classic or non-classic infantile-onset Pompe disease), and the GAA activity levels in the subject are increased to more than about 2% more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal GAA activity levels (e.g., more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal GAA activity levels). In a specific example, a subject has infantile-onset Pompe disease (e.g., classic or non-classic infantile-onset Pompe disease), and the GAA activity levels in the subject are increased to more than about 40%, more than about 45%, more than about 50%, more than about 55%, more than about 60%, more than about 65%, more than about 70%, more than about 75%, more than about 80%, more than about 85%, more than about 90%, or more than about 100% of normal GAA activity levels.

In a specific example, a subject has late-onset Pompe disease, and the GAA activity levels in the subject are increased to more than about 2% more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal GAA activity levels (e.g., more than about 40%, more than about 45%, more than about 50%, more than about 55%, more than about 60%, more than about 65%, more than about 70%, more than about 75%, more than about 80%, more than about 85%, more than about 90%, or more than about 100% of normal GAA activity levels).

In some methods, the method results in increased expression of the multidomain therapeutic protein in the subject (e.g., neonatal subject) compared to a method comprising administering an episomal expression vector encoding the polypeptide of interest in a control subject. In some methods, the method results in increased serum levels of the multidomain therapeutic protein in the subject (e.g., neonatal subject) compared to a method comprising administering an episomal expression vector encoding the polypeptide of interest to a control subject.

In some methods, the method increases expression or activity of the multidomain therapeutic protein or GAA over the subject’s (e.g., neonatal subject’s) baseline expression or activity of the multidomain therapeutic protein or GAA (i.e., any percent change in expression that is larger than typical error bars). In some methods, the method results in expression of the multidomain therapeutic protein or GAA at a detectable level above zero, e.g., at a statistically significant level, a clinically relevant level .

Some methods comprise achieving a durable or sustained effect in a human, such as an at least at least 8 weeks, at least 24 weeks, for example, at least 1 year, or optionally at least 2 year effect, and in some embodiments, at least 3 year, at least 4 year, or at least 5 year effect. Some methods comprise achieving the therapeutic effect in a human in a durable and sustained manner, such as an at least 8 weeks, at least 24 weeks, for example, at least 1 year, or optionally at least 2 year effect, and in some embodiments, at least 3 year, at least 4 year, or at least 5 year effect. In some methods, the increased multidomain therapeutic protein or GAA activity and/or expression level in a human is stable for at least at least 8 weeks, at least 24 weeks, for example, at least 1 year, optionally at least 2 years, and in some embodiments, at least 3 years, at least 4 years, or at least 5 years. In some methods, a steady-state activity and/or level of multidomain therapeutic protein or GAA in a human is achieved by at least 7 days, at least 14 days, or at least 28 days, optionally at least 56 days, at least 80 days, or at least 96 days. In additional methods, the method comprises maintaining multidomain therapeutic protein or GAA activity and/or levels after a single dose in a human for at least 8 weeks, at least 16 weeks, or at least 24 week, or in some embodiments at least 1 year, or at least 2 years, optionally at least 3 years, at least 4 years, or at least 5 years. For example, expression of the multidomain therapeutic protein or GAA can be sustained in the human subject for at least about 8 weeks, at least about 12 weeks, at least about 24 weeks, in certain embodiments, at least about 1 year, or at least about 2 years after treatment, and in some embodiments, at least 3 years, at least 4 years, or at least 5 years after treatment. Likewise, activity of the multidomain therapeutic protein or GAA can be sustained in the human subject for at least about 8 weeks, at least about 12 weeks, at least about 24 weeks, in certain embodiments for at least about 1 year, or at least about 2 years after treatment, and in some embodiments, at least 3 years, at least 4 years, or at least 5 years after treatment. In some methods, expression or activity of the multidomain therapeutic protein or GAA is maintained at a level higher than the expression or activity of the multidomain therapeutic protein or GAA prior to treatment (i.e., the subject’s baseline). In some methods, expression or activity of the multidomain therapeutic protein or GAA is considered sustained if it is maintained at a therapeutically effective level of expression or activity. Relative durations, in other organisms, are understood based, e.g., on life span and developmental stages, are covered within the disclosure above. In some methods, expression or activity of the multidomain therapeutic protein or GAA is considered “sustained” if the expression or activity in a human at six months after administration, one year after administration, or two years after administration, the expression or activity is at least 50% of the expression or activity of the peak level of expression or activity measured for that subject. In certain embodiments, at six months, e.g., at 24 weeks to 28 weeks, after administration the expression or activity is at least 50%, 55%, 60%, 65%, 70%, 75% or 80% of the expression or activity of the peak level of expression or activity measured for that subject. In certain embodiments, at one year, i.e., about 12 months, e.g., at 11-13 months, after administration the expression or activity is at least 50%, 55%, 60%, 65%, 70%, 75% or 80% of the expression or activity of the peak level of expression or activity measured for that subject. In certain embodiments, at two years, i.e., about 24 months, e.g., at 23-25 months, after administration the expression or activity is at least 50%, 55%, 60%, 65%, 70%, 75% or 80% of the expression or activity of the peak level of expression or activity measured for that subject. In certain embodiments, at six months after administration the expression or activity is at least 50%, preferably at least 60% of the expression or activity of the peak level of expression or activity measured for that subject. In certain embodiments, at one year after administration the expression or activity is at least 50%, preferably at least 60% of the expression or activity of the peak level of expression or activity measured for that subject. In certain embodiments, at two years after administration the expression or activity is at least 50%, preferably at least 60% of the expression or activity of the peak level of expression or activity measured for that subject. In preferred embodiments, the subject has routine monitoring of expression or activity levels of the polypeptide, e.g., weekly, monthly, particularly early after administration, e.g., within the first six months. Periodic measurements may establish that the effect on expression or activity is sustained at, e.g. 6 months after administration, one year after administration, or two years after administration. In some methods in neonatal subjects, the expression of the multidomain therapeutic protein or GAA is sustained when the neonatal subject becomes an adult. In some methods, the expression of the multidomain therapeutic protein or GAA is sustained for the lifetime of the subject or neonatal subject.

In some methods, the expression or activity of the multidomain therapeutic protein is at least 50% of the expression or activity of the multidomain therapeutic protein at a peak level of expression measured for the subject at 24 weeks after the administering. In some methods, the expression or activity of the multidomain therapeutic protein is at least 50% of the expression or activity of the multidomain therapeutic protein at a peak level of expression measured for the subject at one year after the administering. In some methods, the expression or activity of the multidomain therapeutic protein is at least 60% of the expression or activity of the multidomain therapeutic protein at a peak level of expression measured for the subject at 24 weeks after the administering. In some methods, the expression or activity of the multidomain therapeutic protein is at least 50% of the expression or activity of the multidomain therapeutic protein at a peak level of expression measured for the subject at two years after the administering. In some methods, the expression or activity of the multidomain therapeutic protein is at least 60% of the expression or activity of the multidomain therapeutic protein at a peak level of expression measured for the subject at 2 years after the administering. In some methods, the expression or activity of the multidomain therapeutic protein is at least 60% of the expression or activity of the multidomain therapeutic protein at a peak level of expression measured for the subject at 24 weeks after the administering.

In some methods involving insertion into an ALB locus, the subject’s circulating albumin levels or cell’s albumin levels are normal. Such methods may comprise maintaining the subject’s circulating albumin levels or the cell’s albumin levels within ±5%, ±10%, ±15%, ±20%, or ±50% of normal circulating albumin levels or normal albumin levels. In some methods, the subject’s or cell’s albumin levels are unchanged as compared to the albumin levels of untreated individuals by at least week 4, at least week 8, at least week 12, or at least week 20. In some methods, the subject’s or cell’s albumin levels transiently drop and then return to normal levels. In particular, the methods may comprise detecting no significant alterations in levels of plasma albumin.

In some methods, the method further comprises assessing preexisting anti-GAA immunity in a subject prior to administering any of the nucleic acid constructs described herein. For example, such methods could comprise assessing immunogenicity using a total antibody (TAb) immune assay or a neutralizing antibody (NAb) assay. In some methods, the subject has not previously been administered recombinant GAA protein.

In some methods, the method further comprises assessing preexisting anti-AAV (e.g., anti-AAV8) immunity in a subject prior to administering any of the nucleic acid constructs described herein. For example, such methods could comprise assessing immunogenicity using a total antibody (TAb) immune assay or a neutralizing antibody (NAb) assay. See, e.g., Manno et al. (2006) Nat. Med. 12(3):342-347, Kruzik et al. (2019) Mol. Ther. Methods Clin. Dev. 14:126-133, and Weber (2021) Front. Immunol. 12:658399, each of which is herein incorporated by reference in its entirety for all purposes. In some embodiments, TAb assays look for antibodies that bind to the AAV vector, whereas NAb assays assess whether the antibodies that are present stop the AAV vector from transducing target cells. With TAb assays, the drug product or an empty capsid can be used to capture the antibodies; NAb assays can require a reporter vector (e.g., a version of the AAV vector encoding luciferase).

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

TABLE 9

Description of Sequences

| SEQ ID NO | Type | Description |
|---|---|---|
| 1 | RNA | Cas9 mRNA |
| 2 | RNA | Cas9 mRNA CDS |
| 3 | DNA | Cas9 CDS |
| 4 | DNA | Human ALB Intron 1 |
| 5 | DNA | Guide RNA Target Sequence Plus PAM v1 |
| 6 | DNA | Guide RNA Target Sequence Plus PAM v2 |
| 7 | DNA | Guide RNA Target Sequence Plus PAM v3 |
| 8 | Protein | SpCas9 Protein V1 |
| 9 | DNA | SpCas9 DNA V1 |
| 10 | DNA | SpCas9 mRNA (cDNA) |
| 11 | Protein | SpCas9 Protein V2 |
| 12 | RNA | SpCas9 mRNA V2 |
| 13 | Protein | SV40 NLS v1 |
| 14 | Protein | SV40 NLS v2 |
| 15 | Protein | Nucleoplasmin NLS |
| 16 | RNA | crRNA Tail v1 |
| 17 | RNA | crRNA Tail v2 |
| 18 | RNA | TracrRNA v1 |
| 19 | RNA | TracrRNA v2 |
| 20 | RNA | TracrRNA v3 |
| 21 | RNA | gRNA Scaffold v1 |
| 22 | RNA | gRNA Scaffold v2 |
| 23 | RNA | gRNA Scaffold v3 |
| 24 | RNA | gRNA Scaffold v4 |
| 25 | RNA | gRNA Scaffold v5 |
| 26 | RNA | gRNA Scaffold v6 |
| 27 | RNA | gRNA Scaffold v7 |
| 28 | RNA | gRNA Scaffold v8 |
| 29 | RNA | Modified gRNA Scaffold |
| 30-61 | RNA | Human ALB Intron 1 Guide Sequences |
| 62-125 | RNA | Human ALB Intron 1 sgRNA Sequences |
| 126-157 | DNA | Human ALB Intron 1 Guide RNA Target Sequences |
| 158 | DNA | ITR 145 |
| 159 | DNA | ITR 141 |
| 160 | DNA | ITR 130 |
| 161 | DNA | SV40 polyA |
| 162 | DNA | bGH polyA |
| 163 | DNA | Mouse Alb exon 2 Splice Acceptor |
| 164 | RNA | Mouse Alb Intron 1 Guide Sequence g666 |
| 165 | DNA | Mouse Alb Intron 1 Guide RNA Target Sequence g666 |
| 166-167 | RNA | Mouse Alb Intron 1 sgRNA Sequences g666 |
| 168 | DNA | ITR 145 Reverse Complement |
| 169 | DNA | SV40 polyA v2 |
| 170 | Protein | Human GAA Protein (NP 000143.2) |
| 171 | DNA | Human GAA cDNA/mRNA (NM 000152.5) |
| 172 | DNA | Human GAA CDS (CCDS32760.1) |
| 173 | Protein | Human GAA (70-952) Protein |
| 174 | DNA | Human GAA (70-952) CDS |
| 175 | DNA | Human GAA (70-952) CDS - DC-0 |
| 176 | DNA | Human GAA (70-952) CDS - GA-0 |
| 177 | DNA | Human GAA (70-952) CDS - GS-0 |
| 178 | DNA | Human GAA (70-952) CDS - GS-0v2 |
| 179 | DNA | Human GAA (70-952) CDS - GS-1 |
| 180 | DNA | Human GAA (70-952) CDS - GS-44 |
| 181 | DNA | Human GAA (70-952) CDS - GS-50 |
| 182 | DNA | Human GAA (70-952) CDS - RE-8 |
| 183 | Protein | 12450 anti-CD63 scFv |
| 184 | DNA | 12450 anti-CD63 scFv CDS |
| 185 | DNA | 12450 anti-CD63 scFv CDS - DC-0 |
| 186 | DNA | 12450 anti-CD63 scFv CDS - GA-0 |
| 187 | DNA | 12450 anti-CD63 scFv CDS - GS-0 |
| 188 | DNA | 12450 anti-CD63 scFv CDS - GS-0v2 |
| 189 | DNA | 12450 anti-CD63 scFv CDS - GS-1 |
| 190 | DNA | 12450 anti-CD63 scFv CDS - GS-44 |
| 191 | DNA | 12450 anti-CD63 scFv CDS - GS-50 |
| 192 | DNA | 12450 anti-CD63 scFv CDS - RE-8 |
| 193 | Protein | 12450 anti-CD63 scFv:GAA (70-952) Fusion Protein |
| 194 | DNA | 12450 anti-CD63 scFv:GAA (70-952) Fusion Protein CDS |
| 195 | DNA | 12450 anti-CD63 scFv:GAA (70-952) Fusion Protein CDS - DC-0 |
| 196 | DNA | 12450 anti-CD63 scFv:GAA (70-952) Fusion Protein CDS - GA-0 |
| 197 | DNA | 12450 anti-CD63 scFv:GAA (70-952) Fusion Protein CDS - GS-0 |
| 198 | DNA | 12450 anti-CD63 scFv:GAA (70-952) Fusion Protein CDS - GS-0v2 |
| 199 | DNA | 12450 anti-CD63 scFv:GAA (70-952) Fusion Protein CDS - GS-1 |
| 200 | DNA | 12450 anti-CD63 scFv:GAA (70-952) Fusion Protein CDS - GS-44 |
| 201 | DNA | 12450 anti-CD63 scFv:GAA (70-952) Fusion Protein CDS - GS-50 |
| 202 | DNA | 12450 anti-CD63 scFv:GAA (70-952) Fusion Protein CDS - RE-8 |
| 203 | DNA | Glycine-Encoding Sequence v1 |
| 204 | DNA | Glycine-Encoding Sequence v2 |
| 205 | DNA | Human GAA (70-952) CDS - 1 - DC |
| 206 | DNA | Human GAA (70-952) CDS - 1 - GS |
| 207 | DNA | Human GAA (70-952) CDS - 2 - DC |
| 208 | DNA | Human GAA (70-952) CDS - 2 - GS |
| 209 | DNA | Human GAA (70-952) CDS - 3 - DC |
| 210 | DNA | Human GAA (70-952) CDS - 3 - GS |
| 211 | DNA | Human GAA (70-952) CDS - 4 - DC |
| 212 | DNA | Human GAA (70-952) CDS - 4 - GS |
| 213 | Protein | GAA 1 77 |
| 214 | Protein | GAA 2 22 |
| 215 | Protein | GAA 3 61 |
| 216-537 | DNA & Protein | Domains in Anti-hTfR Antibodies, Antigen-Binding Fragments or scFv Molecules |
| 538-569 | Protein | Anti-hTfR scFvs |
| 570 | Protein | 12799B-2×G4S-GAA |
| 571 | Protein | 12839B-2×G4S-GAA |
| 572 | Protein | 12843B-2×G4S-GAA |
| 573 | Protein | 12847B-2×G4S-GAA |
| 574 | DNA | 12799B-2×G4S-GAA |
| 575 | DNA | 12839B-2×G4S-GAA |
| 576 | DNA | 12843B-2xG4S-GAA |
| 577 | DNA | 12847B-2×G4S-GAA |
| 578-580 | DNA | Optimized 12799B-2×G4S-GAA |
| 581-583 | DNA | Optimized 12843B-2xG4S-GAA |
| 584-586 | DNA | Optimized 12847B-2xG4S-GAA |
| 587 | DNA | 12799 GA 0 anti-TfR scFv |
| 588 | DNA | 12799 GS 0 anti-TfR scFv |
| 589 | DNA | 12799 GS 0v2 anti-TfR scFv |
| 590 | DNA | 12843 GA 0 anti-TfR scFv |
| 591 | DNA | 12843 GS 0 anti-TfR scFv |
| 592 | DNA | 12843 GS 0v2 anti-TfR scFv |
| 593 | DNA | 12847 GA 0 anti-TfR scFv |
| 594 | DNA | 12847 GS 0 anti-TfR scFv |
| 595 | DNA | 12847 GS 0v2 anti-TfR scFv |
| 596 | DNA | 12799 anti-TfR scFv |
| 597 | DNA | 12839 anti-TfR scFv |
| 598 | DNA | 12843 anti-TfR scFv |
| 599 | DNA | 12847 anti-TfR scFv |
| 600 | Protein | Linker |
| 601 | Protein | Kappa Constant Light Domain |
| 602 | Protein | IgG1 CH1 Heavy Domain |
| 603-672 | Protein | Fab Heavy and Light Chains |
| 673 | Protein | Mus musculus Ror1 Signal Peptide |
| 674 | Protein | IgG4 CH1 Heavy Domain |
| 675-706 | Protein | Additional anti-TfR scFv:GAA Sequences |
| 707 | DNA | 12837scfv-GAA-1 0CpG 27 |
| 708 | DNA | 12837scfv-GAA-2 5CpG 27 |
| 709 | Protein | Optional N-Terminal Sequence |
| 710 | DNA | ITR 141 Reverse Complement |
| 711 | DNA | ITR 130 Reverse Complement |
| 712 | DNA | SV40 polyA V3 |
| 713 | Protein | 3X G4S Linker |
| 714 | Protein | 2X G4S Linker |
| 715-719 | DNA | 3X G4S Linker Coding Sequences |
| 720-726 | DNA | 2X G4S Linker Coding Sequences |
| 727 | DNA | 1X G4S Linker Coding Sequence |
| 728 | DNA | pINT ITR130 12843 GAA native SV40pA |
| 729 | DNA | pINT ITR130 12843scFv:GAA 0CpG v0 (GA 0) |
| 730 | DNA | pINT ITR130 12843scFv:GAA 0CpG v1 (GS 0 v1) |
| 731 | DNA | pINT ITR130 12843scFv:GAA 0CpG v2 (GS 0 v2) |
| 732 | DNA | pINT ITR130 12847scFv:GAA native |
| 733 | DNA | pINT ITR130 12847scFv:GAA 0CpG v0 (GA 0) |
| 734 | DNA | pINT ITR130 12847scFv:GAA 0CpG v1 (GS 0 v1) |
| 735 | DNA | pINT ITR130 12847scFv:GAA 0CpG v2 (GS 0 v2) |
| 736 | DNA | pINT-ITR130-Anti-CD63:GAA GA 0 |

### EXAMPLES

### Example 1. Development of System for Neonatal Insertion Into Albumin Locus in Liver

A system for nuclease-mediated insertion (e.g., CRISPR/Cas) of a transgene into a specific locus (e.g., albumin intron 1) was developed to produce durable expression of the transgene, including when administered to neonates. Exemplary components of the system, including those used in subsequent examples, are described in more detail below.

### Single Guide RNA Design and Selection

The ALB locus was selected as the insertion site for the DNA templates. A list of single guide RNAs (sgRNAs) was generated that target human ALB intron 1. See Table 10. Candidate sgRNAs were synthesized and formulated into lipid nanoparticles (LNPs) with Cas9 mRNA for evaluation in vitro and in vivo.

TABLE 10

Human ALB Intron 1 Guide RNAs

| Guide RNA | SEQ ID NO (DNA-Targeting Segment) | SEQ ID NO (Unmodified sgRNA) | SEQ ID NO (Modified sgRNA) | SEQ ID NO (Guide RNA Target Sequence) |
|---|---|---|---|---|
| G009844 | 30 | 62 | 94 | 126 |
| G009851 | 31 | 63 | 95 | 127 |
| G009852 | 32 | 64 | 96 | 128 |
| G009857 | 33 | 65 | 97 | 129 |
| G009858 | 34 | 66 | 98 | 130 |
| G009859 | 35 | 67 | 99 | 131 |
| G009860 | 36 | 68 | 100 | 132 |
| G009861 | 37 | 69 | 101 | 133 |
| G009866 | 38 | 70 | 102 | 134 |
| G009867 | 39 | 71 | 103 | 135 |
| G009868 | 40 | 72 | 104 | 136 |
| G009874 | 41 | 73 | 105 | 137 |
| G012747 | 42 | 74 | 106 | 138 |
| G012748 | 43 | 75 | 107 | 139 |
| G012749 | 44 | 76 | 108 | 140 |
| G012750 | 45 | 77 | 109 | 141 |
| G012751 | 46 | 78 | 110 | 142 |
| G012752 | 47 | 79 | 111 | 143 |
| G012753 | 48 | 80 | 112 | 144 |
| G012754 | 49 | 81 | 113 | 145 |
| G012755 | 50 | 82 | 114 | 146 |
| G012756 | 51 | 83 | 115 | 147 |
| G012757 | 52 | 84 | 116 | 148 |
| G012758 | 53 | 85 | 117 | 149 |
| G012759 | 54 | 86 | 118 | 150 |
| G012760 | 55 | 87 | 119 | 151 |
| G012761 | 56 | 88 | 120 | 152 |
| G012762 | 57 | 89 | 121 | 153 |
| G012763 | 58 | 90 | 122 | 154 |
| G012764 | 59 | 91 | 123 | 155 |
| G012765 | 60 | 92 | 124 | 156 |
| G012766 | 61 | 93 | 125 | 157 |

LNPs were first screened in primary human hepatocytes (PHH) using a bidirectional nanoluc-encoding AAV insertion template as a reporter. LNPs that supported targeted insertion of nanoluc were identified by measuring nanoluc protein secreted into the supernatant of PHH cultures. Candidates that passed initial PHH screening were then tested for their ability to support in vivo gene insertion. Top candidates from in vivo studies were functionally evaluated for off-target cutting.

LNP-g9860, which is formulated with ALB-targeting sgRNA 9860, described in more detail below, was selected based on supporting robust transgene expression levels across multiple platforms (primary human and non-human primate hepatocytes, ALB humanized mice, and non-human primates), lack of confirmed off-target sites, translation across species, lack of common human SNPs in the target site, low variability of transgene expression within groups, and performance across a dose range. The target site of sgRNA 9860 is conserved in cynomolgus monkeys. LNP-g9860 had no detectable off-target sites in the human genome (targeted amplicon sequencing performed in two lots of primary human hepatocytes at saturating levels of editing failed to validate any locus other than on-target at ALB) and supported transgene expression via insertion in primary human and non-human primate hepatocytes, ALB humanized mice, and non-human primates.

### LNP-g9860

LNP-g9860 was developed for use in targeting human ALB intron 1. LNP-g9860 is a lipid nanoparticle that includes a sgRNA of 100 nucleotides in length (g9860) and Cas9-encoding mRNA, each of which is described further below, encapsulated in an LNP comprised of four different lipids. The Cas9 protein, expressed from the Cas9 mRNA, is directed to cleave the DNA when sgRNA 9860 binds to the targeted complementary DNA sequence associated with a PAM. The composition of the LNP is summarized in Table 11. LNP-g9860 comprises four lipids at the following molar ratios: 50 mol% Lipid A, 9 mol% DSPC, 38 mol% cholesterol, and 3 mol% PEG2k-DMG and is formulated in aqueous buffer composed of 50 mM Tris-HCl, 45 mM NaCl, 5% (w/v) sucrose, at pH 7.4. The N:P ratio is about 6, and the gRNA:Cas9 mRNA ratio is about 1:2 by weight.

TABLE 11

Lipid Nanoparticle (LNP-g9860) Composition

| | |
|---|---|
| Component | Description |
| Active Pharmaceutical Components | Cas9 mRNA |
| | sgRNA (gRNA9860) |
| Lipid Excipients | Lipid A: (9Z,12Z)-3-((4,4-bis(octyloxy)butanoyl)oxy)-2-((((3-(diethylamino)propoxy)carbonyl)oxy)methyl)propyl octadeca-9,12-dienoate |
| | Cholesterol |
| | DSPC |
| | PEG2K-DMG |
| Other Excipients | Tris, NaCl, Sucrose |
| | WFI |

Single guide RNA. The single guide RNA (sgRNA 9860) used in LNP-g9860 is a 100-mer oligonucleotide containing a 20-nucleotide sequence that is complementary to the target region in intron 1 of the human ALB gene. The target sequence recognized by g9860 is conserved in the cynomolgus monkey mfAlb gene intron 1. The sequence for g9860 is set forth in SEQ ID NOs: 68 and 100. Chemical modifications are incorporated into the 100-mer during synthesis, which include phosphorothioate (PS) linkages at the 5′- and 3′-end of the sgRNA and 2′-O-methyl modifications to some of the sugars of the RNA.

Cas9 mRNA. The Cas9 messenger RNA (mRNA) used in LNP-g9860 is based on the Cas9 protein sequence from Streptococcus pyogenes. The Cas9-encoding mRNA (SEQ ID NO: 1, with a coding sequence (CDS) set forth in SEQ ID NO: 2), is approximately 4400 nucleotides in length. The sequence contains a 5′ cap, a 5′ untranslated region (UTR), an open reading frame (ORF) encoding the Cas9 protein, a 3′ UTR, and a polyA tail. The 5′ cap is generated co-transcriptionally by use of a synthetic cap analogue structure, known as anti-reverse cap analogue (ARCA). The uracils in the mRNA sequence have been completely replaced by a modified N¹ methylpseudouridine during the in vitro transcription. The 5′ end of the mRNA has a synthetic cap analog structure. The poly-A tail is approximately 100 nucleotides.

### LNP-g666

LNP-g666 was developed for use in targeting mouse Alb intron 1. LNP-g666 is the same as LNP-g9860, except human-albumin-targeting g9860 is replaced with g666, a guide RNA targeting mouse albumin intron 1. The sequence for g666 is set forth in SEQ ID NOS: 166 and 167.

### rAAV8 Vector

A recombinant AAV8 (rAAV8) vector was developed to carry the DNA insertion templates. The rAAV8 vector carrying the DNA insertion templates is a non-replicating vector that is an AAV-based vector derived from AAV serotype 8. The genome is a single-stranded deoxyribonucleic acid (DNA), comprising inverted terminal repeats (ITR) at each end. The ITRs flank the promoterless insertion template. The AAV ITRs flanking the cassette were derived from AAV2. The DNA insertion templates delivered by rAAV8 vector can be designed as promoterless templates, thus relying on the targeted ALB locus promoter for expression.

### Example 2. Durable Human FIX Protein Expression After Insertion in Neonatal Mice

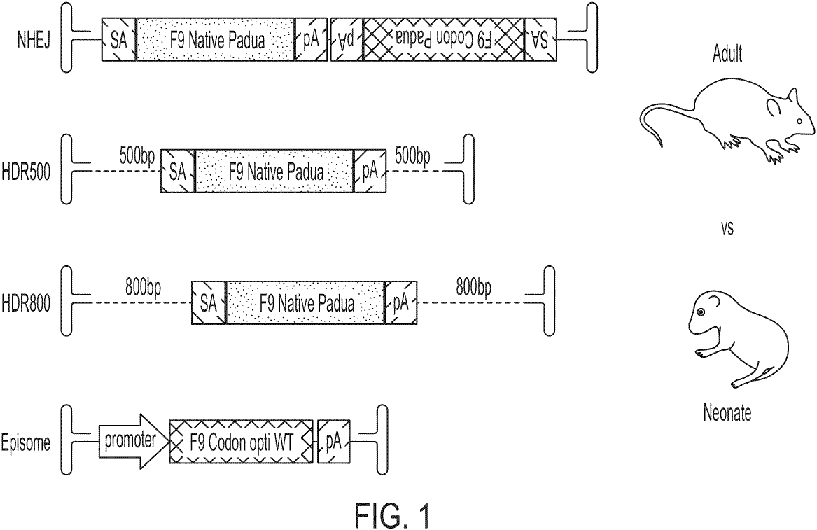

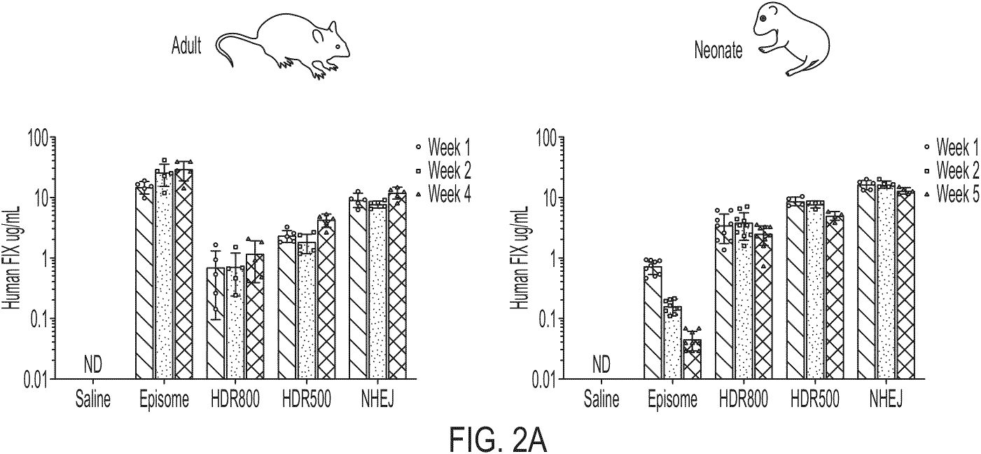

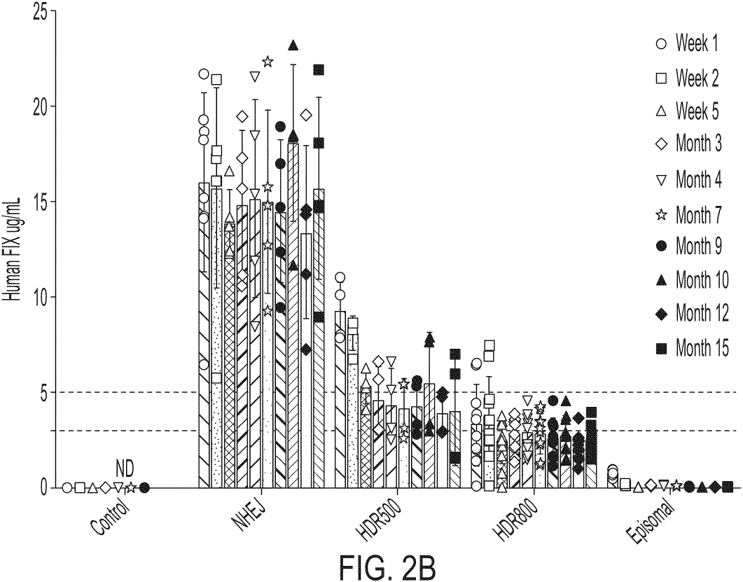

To compare episome-mediated expression versus insertion-mediated expression in adult and neonatal mice, and to compare different DNA repair pathways in adult and neonatal mice, we compared hFIX serum levels following administration of a hFIX episome (expression driven by hAAT promoter), a bidirectional hFIX NHEJ insertion template, a hFIX HDR insertion template with homology arms of 500 bp, and a hFIX HDR insertion template with homology arms of 800 bp. See FIG. 1. Neonatal C57BL/6 mice were dosed at P0 or P1 with the following: (1) 4 mg/kg of LNP-g666 and 3e9 vg/mouse of rAAV8 with the hFIX-HDR-500 template; (2) 4 mg/kg of LNP-g666 and 3e9 vg/mouse of rAAV8 with the hFIX-HDR-800 template; (3) 4 mg/kg of LNP-g666 and 3e9 vg/mouse of rAAV8 with the hFIX-NHEJ template; or (4) 3e9 vg/mouse of rAAV8 episomal template. Saline-injected mice were used as a negative control. The hFIX coding sequence in the episomal AAV was a codon-optimized sequence encoding wild type human F9. The hFIX coding sequence in the two HDR constructs was the native human F9 coding sequence with the Padua mutation (R338L). Blood was collected and plasma prepared at 1 week, 2 weeks, and 5 weeks post-dosing. hFIX levels were measured by human FIX ELISA. The experiment was then repeated in adult C57BL/6 mice, with the adult mice being dosed with the following: (1) 0.8 mg/kg of LNP-g666 and 2e10 vg/mouse of rAAV8 with the hFIX-HDR-500 template; (2) 0.8 mg/kg of LNP-g666 and 2e10 vg/mouse of rAAV8 with the hFIX-HDR-800 template; (3) 0.8 mg/kg of LNP-g666 and 2e10 vg/mouse of rAAV8 with the hFIX-NHEJ template; or (4) 2e10 vg/mouse of rAAV8 episomal template. Saline-injected mice were used as a negative control. Blood was collected and plasma prepared at 1 week, 2 weeks, and 4 weeks post-dosing. The results are shown in FIGS. 2A-2B and Tables 12-14. Episome-mediated expression was low even at the first time point compared to insertion-mediated expression in neonates and was lost over time in neonates. The opposite was observed in adult mice: episome-mediated expression was higher at the first time point and subsequent time points compared to insertion-mediated expression in adult mice. These results confirmed what was observed in a previous similar experiment (data not shown). In contrast to the results in the neonatal mice, hFIX levels stayed steady in adult mice with both episomal and insertion constructs, with the episomal construct giving the highest expression. See FIGS. 2A-2B and Tables 12-14.

TABLE 12

Human FIX Serum Levels (µg/mL) in Neonatal Mice
Episome

| | | | | | | | | | |
|---|---|---|---|---|---|---|---|---|---|
| W1 | 0.9 | 0.56 | 0.48 | 0.52 | 0.96 | 0.67 | 0.93 | 0.98 | 0.77 |
| W2 | 0.2 | 0.11 | 0.12 | 0.14 | 0.15 | 0.17 | 0.22 | 0.21 | 0.15 |
| W5 | 0.06 | 0.03 | 0.03 | 0.07 | 0.07 | 0.03 | 0.04 | 0.04 | 0.04 |

NHEJ

| | | | | | |
|---|---|---|---|---|---|
| W1 | 14.22 | 19.28 | 14.12 | 18.67 | 21.69 |
| W2 | 16.08 | 17.25 | 16.07 | 17.66 | 21.39 |
| W5 | 12.29 | 12.41 | 14.18 | 16.61 | 13.71 |

HDR500

| | | | | |
|---|---|---|---|---|
| W1 | 11.03 | 8.02 | 7.86 | 10.11 |
| W2 | 8.29 | 6.74 | 8.63 | 8.61 |
| W5 | 6.27 | 4.09 | 5.24 | 5.49 |

HDR800

| | | | | | | | | | | |
|---|---|---|---|---|---|---|---|---|---|---|
| W1 | 3.86 | 6.44 | 6.52 | 4.43 | | 1.41 | 2 | 3.58 | 2.71 | 2.07 |
| W2 | 3.54 | 6.9 | 7.46 | 4.44 | 4.64 | 1.65 | 2.45 | 3.55 | 2.83 | 2.5 |
| W5 | 2.4 | 3.49 | 3.48 | 3.33 | 3.77 | 0.77 | 2.19 | 2.7 | 2.5 | 1.68 |

TABLE 13

Human FIX Serum Levels (µg/mL) in Adult Mice
Episome

| W1 | 16.69 | 15.57 | 18.97 | 14.17 | 9.82 |
|---|---|---|---|---|---|
| W2 | 25.39 | 23.07 | 41.53 | 27.53 | 12.07 |
| W4 | 39.97 | 26.61 | 39.63 | 28.5 | 14.28 |
| | NHEJ | | | | |
| W1 | 8.1 | 9.52 | 13.09 | 6.55 | 9.2 |
| W2 | 9.15 | 8.12 | 8.18 | 6.46 | 7.27 |
| W4 | 14.86 | 10.86 | 13.85 | 8.42 | 13 |
| | HDR500 | | | | |
| W1 | 2.23 | 2.5 | 1.89 | 3.14 | 1.85 |
| W2 | 1.2 | 2.62 | 1.55 | 2.41 | 1.34 |
| W4 | 3.83 | 5.39 | 4.89 | 4.62 | 2.75 |
| | HDR800 | | | | |
| W1 | 1.65 | 0.94 | 0.26 | 0.54 | 0.14 |
| W2 | 0.69 | 1.52 | 0.24 | 0.67 | 0.46 |
| W4 | 2.13 | 1.87 | 0.49 | 0.94 | 0.43 |

TABLE 14

Human FIX Serum Levels (µg/mL) in Neonatal Mice

| Mice # | Group # | Wk 1 hF9 ug/mL | Wk 2 hF9 ug/mL | Wk 5 hF9 ug/mL | Month 3 hF9 ug/mL | Month 4 hF9 ug/mL | Month 7 hF9 ug/mL | Month 9 hF9 ug/mL | Month 10 hF9 ug/mL | Month 12 hF9 ug/mL | Month 15 hF9 ug/mL |
|---|---|---|---|---|---|---|---|---|---|---|---|
| 1 | Ctrl 1 | ND | ND | ND | ND | ND | ND | ND | ND | ND | ND |
| 2 | Ctrl 2 | ND | ND | ND | ND | ND | ND | ND | ND | ND | ND |
| 3 | Ctrl 3 | ND | ND | ND | ND | ND | ND | ND | ND | ND | ND |
| 4 | Ctrl 4 | ND | ND | ND | ND | ND | ND | ND | ND | ND | ND |
| 5 | Ctrl 5 | ND | ND | ND | ND | ND | ND | ND | ND | ND | ND |
| 6 | Ctrl 6 | ND | ND | ND | ND | ND | ND | ND | ND | ND | ND |
| 7 | Ctrl 7 | ND | ND | ND | ND | ND | ND | ND | ND | ND | ND |
| 8 | Ctrl 8 | ND | ND | ND | ND | ND | ND | ND | ND | ND | ND |
| 9 | HDR500-1 | 11.03 | 8.29 | 6.27 | 3.04 | 3.12 | 3.13 | 3.32 | 3.35 | 2.91 | 1.60 |
| 10 | HDR500-3 | 8.02 | 6.74 | 4.09 | 3.05 | 2.51 | 2.59 | 2.80 | 2.98 | 2.95 | 1.54 |
| 11 | HDR500-4 | 7.86 | 8.63 | 5.24 | 6.61 | 5.11 | 5.44 | 5.62 | 7.64 | 4.76 | 5.97 |
| 12 | HDR500-5 | 10.11 | 8.61 | 5.49 | 5.68 | 6.60 | 5.43 | 5.33 | 7.89 | 5.00 | 7.00 |
| 13 | Episomal 1 | 0.90 | 0.20 | 0.06 | 0.16 | 0.14 | 0.12 | 0.04 | 0.05 | 0.04 | 0.04 |
| 14 | Episomal 2 | 0.56 | 0.11 | 0.03 | 0.09 | 0.09 | 0.08 | 0.03 | 0.03 | 0.04 | 0.06 |
| 15 | Episomal 3 | 0.48 | 0.12 | 0.03 | 0.11 | 0.09 | 0.09 | 0.02 | 0.04 | 0.02 | 0.03 |
| 16 | Episomal 4 | 0.52 | 0.14 | 0.07 | 0.14 | 0.11 | 0.09 | 0.03 | 0.02 | 0.02 | 0.05 |
| 17 | Episomal 5 | 0.96 | 0.15 | 0.07 | NA (mouse died) | | | NA (mouse died) | | | |
| 18 | Episomal 6 | 0.67 | 0.17 | 0.03 | 0.13 | 0.11 | 0.11 | 0.03 | 0.04 | 0.00 | 0.04 |
| 19 | Episomal 7 | 0.93 | 0.22 | 0.04 | 0.13 | 0.09 | 0.09 | 0.09 | 0.05 | 0.04 | 0.03 |
| 20 | Episomal 8 | 0.98 | 0.21 | 0.04 | 0.14 | 0.10 | 0.11 | 0.06 | 0.03 | 0.04 | 0.05 |
| 21 | Episomal 9 | 0.77 | 0.15 | 0.04 | 0.14 | 0.15 | 0.12 | 0.05 | 0.03 | 0.05 | 0.07 |
| 22 | HDR800-1 | 3.86 | 3.54 | 2.40 | 2.08 | 2.26 | 2.91 | 1.66 | 2.09 | 1.98 | 1.77 |
| 23 | HDR800-2 | 6.44 | 6.90 | 3.49 | 3.47 | 3.36 | 3.49 | ND | 2.92 | 2.24 | 2.31 |
| 24 | HDR800-3 | 6.52 | 7.46 | 3.48 | 2.80 | 3.62 | 3.45 | 2.71 | 2.70 | 1.95 | 2.54 |
| 25 | HDR800-4 | 4.43 | 4.44 | 3.33 | 3.59 | 4.55 | 4.14 | 3.22 | 3.75 | 2.51 | 3.28 |
| 26 | HDR800-5 | NA | 4.64 | 3.77 | 3.47 | 3.82 | 4.26 | 4.57 | 4.56 | 3.65 | 3.95 |
| 27 | HDR800-6 | 1.41 | 1.65 | 0.77 | 1.54 | 1.48 | 1.19 | 3.35 | 1.50 | 1.47 | 1.60 |
| 28 | HDR800-7 | 2.00 | 2.45 | 2.19 | 3.32 | 3.11 | 2.26 | 2.37 | 2.75 | 2.63 | 2.29 |
| 29 | HDR800-8 | 3.58 | 3.55 | 2.70 | 2.16 | 2.09 | 2.36 | 1.58 | 2.04 | 1.59 | 2.08 |
| 30 | HDR800-9 | 0.09 | 0.10 | 0.03 | NA (mouse died) | | | NA (mouse died) | | | |
| 31 | HDR800-10 | 2.71 | 2.83 | 2.50 | 3.87 | 3.36 | 3.12 | 2.52 | 3.58 | 2.45 | 2.80 |
| 32 | HDR800-11 | 2.07 | 2.50 | 1.68 | 1.35 | 1.66 | 1.28 | 1.15 | 1.45 | 1.01 | 1.50 |
| 33 | NHEJ 1 | 6.45 | 5.75 | NA (mouse died) | | | | NA (mouse died) | | | |
| 34 | NHEJ 2 | 14.22 | 16.08 | 12.29 | 10.58 | 8.44 | 9.27 | 9.45 | 11.67 | 7.24 | 8.95 |
| 35 | NHEJ 3 | 19.28 | 17.25 | 12.41 | 15.67 | 18.44 | 15.77 | 14.70 | 18.38 | 14.58 | 14.80 |
| 36 | NHEJ 4 | 15.19 | NA (mouse died) | | | | | NA (Mouse died) | | | |
| 37 | NHEJ 5 | 14.12 | 16.07 | 14.18 | 17.28 | 15.39 | 14.79 | 16.99 | 18.47 | 14.32 | 14.68 |
| 38 | NHEJ 6 | 18.67 | 17.66 | 16.61 | 19.46 | 21.53 | 22.32 | 18.93 | 23.21 | 19.53 | 21.90 |
| 39 | NHEJ 7 | 18.23 | NA (mouse died) | | | | | NA (Mouse died) | | | |
| 40 | NHEJ 8 | 21.69 | 21.39 | 13.71 | 11.15 | 11.88 | 12.72 | 12.36 | 18.53 | 11.19 | 18.07 |

These experiments showed that expression of inserted F9 is durable in neonatal livers, indicating that insertion of F9 templates into the albumin locus can result in durable expression in neonatal subjects. These genome integration provided durable expression that was maintained throughout the experiment in neonatal mice.

### Example 3. Development of Neonatal Insertion System and Reagents for Treatment of Pompe Disease

A system for nuclease-mediated insertion (e.g., CRISPR/Cas) of an anti-CD63:GAA transgene or an anti-TfR:GAA transgene into a specific locus (e.g., albumin intron 1) was developed to produce durable expression of anti-CD63:GAA or anti-TfR:GAA, including when administered to neonates.

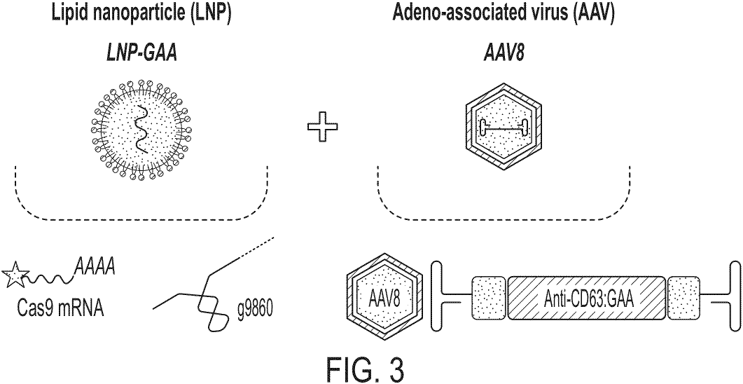

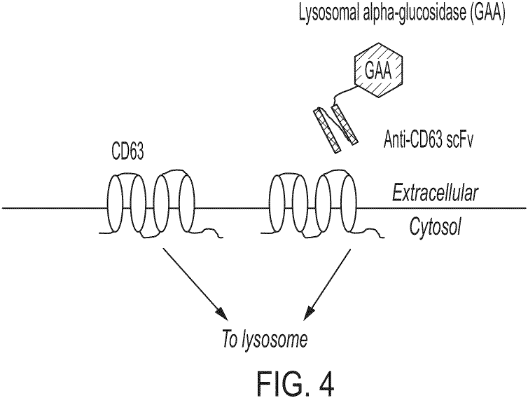

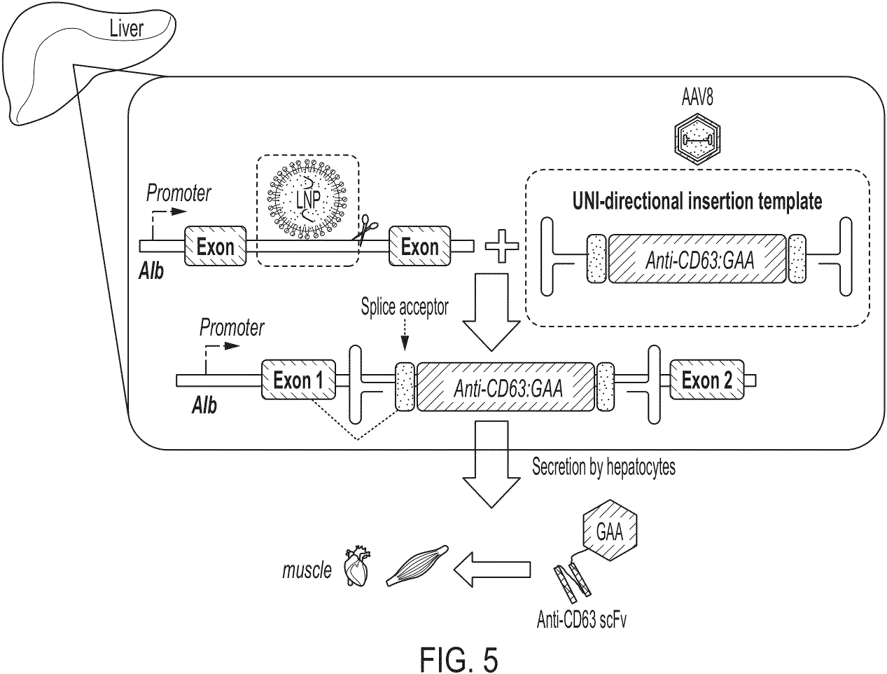

Exemplary components of the system for insertion for anti-CD63:GAA, including those used in subsequent examples, are described in more detail below. See FIGS. 3-5. The anti-CD63:GAA DNA template in the working examples described below is brought into the liver by a recombinant AAV8 vector, and the CRISPR/Cas9 RNA components (Cas9 mRNA and sgRNA) are delivered to the liver by LNP-mediated delivery (FIGS. 3 and 5). The anti-CD63:GAA protein produced by the liver is targeted to lysosomes in the muscle by targeting CD63, which is a rapidly internalizing protein highly expressed in the muscle. See FIG. 4. Single guide RNA, LNP-g9860, Cas9 mRNA, and LNP-g666 design and selection were as described in Example 1.

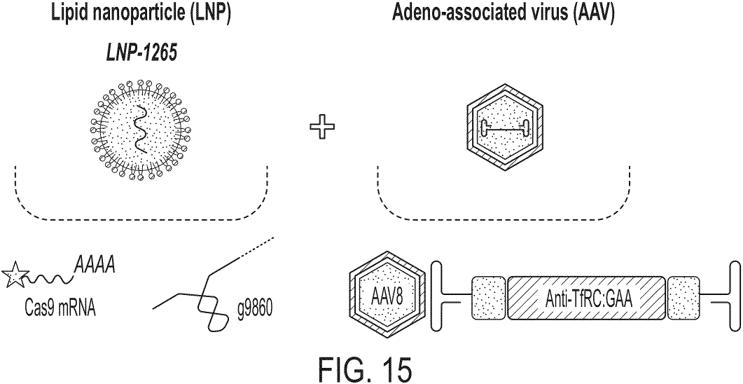

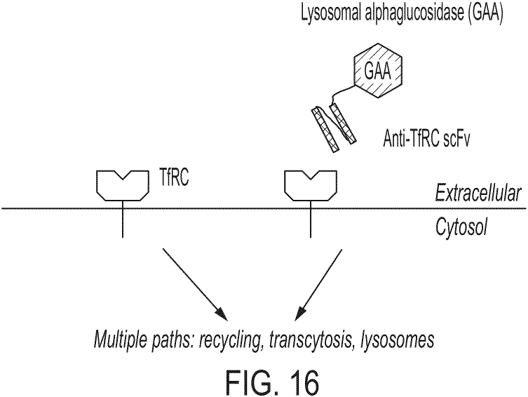

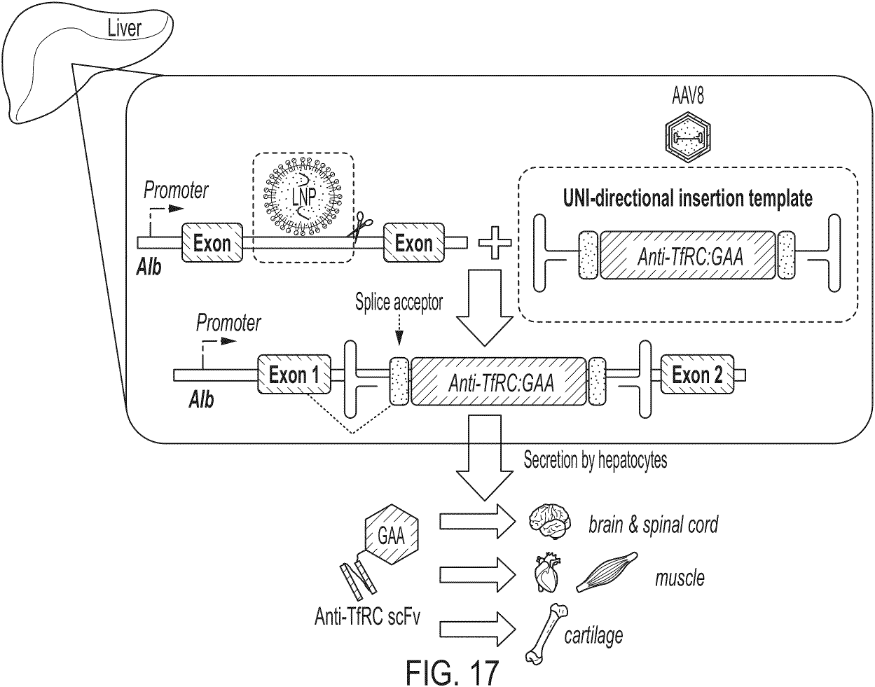

Exemplary components of the system for anti-TfR:GAA, including those used in subsequent examples, are described in more detail below. See FIGS. 15-17. The anti-TfR:GAA DNA templates in the working examples described below are brought into the liver by a recombinant AAV8 vector, and the CRISPR/Cas9 RNA components (Cas9 mRNA and sgRNA) are delivered to the liver by LNP-mediated delivery (FIGS. 15 and 17). The anti-TfR:GAA protein produced by the liver is targeted the muscle and CNS by targeting TfR, which is expressed in muscle and on brain endothelial cells. Transcytosis of TfR in these cells enables blood-brain-barrier crossing. See FIG. 16. Single guide RNA, LNP-g9860, Cas9 mRNA, and LNP-g666 design and selection were as described in Example 1.

### DNA Template Design and Selection

We engineered a DNA template for insertion of a nucleic encoding anti-CD63:GAA fusions in which the C-terminus of a single-chain fragment variable (scFv) is fused to the N-terminus of amino acids 70-952 of GAA with a glycine-serine linker. The GAA (70-952) sequence is set forth in SEQ ID NO: 173 and is encoded by the sequence set forth in SEQ ID NO: 174. The DNA template is set forth in SEQ ID NO: 194 and encodes the fusion protein set forth in SEQ ID NO: 193. A splice acceptor site is encoded upstream of the anti-CD63:GAA transgene, and a polyadenylation sequence is encoded downstream of the anti-CD63:GAA transgene. The splice acceptor sequence at the 5′ end of the transgene was derived from mouse Alb exon 2 splice acceptor. The polyadenylation sequence at the 3′ end of the transgene was derived from simian virus 40 (SV40).

We engineered DNA templates for insertion of a nucleic encoding anti-TfR:GAA fusions in which the C-terminus of a single-chain fragment variable (scFv) is fused to the N-terminus of amino acids 70-952 of GAA with a glycine-serine linker. The GAA (70-952) sequence is set forth in SEQ ID NO: 173 and is encoded by the sequence set forth in SEQ ID NO: 174. A splice acceptor site is encoded upstream of the anti-TfR:GAA transgene, and a polyadenylation sequence is encoded downstream of the anti-TfR:GAA transgene. The splice acceptor sequence at the 5′ end of the transgene was derived from mouse Alb exon 2 splice acceptor. The polyadenylation sequence at the 3′ end of the transgene was derived from simian virus 40 (SV40).

### rAAV8 Vector

A recombinant AAV8 (rAAV8) vector was developed to carry the DNA insertion templates. The rAAV8 vector carrying the anti-CD63:GAA DNA template (REGV044) is a non-replicating vector that is an AAV-based vector derived from AAV serotype 8. The genome is a single-stranded deoxyribonucleic acid (DNA), comprising inverted terminal repeats (ITR) at each end. The ITRs flank the anti-CD63:GAA promoterless insertion template. The AAV ITRs flanking the cassette were derived from AAV2. The anti-CD63:GAA DNA template delivered by rAAV8 vector was designed as a promoterless template, thus relying on the targeted ALB locus promoter for expression.

The rAAV8 vector carrying the anti-TfR:GAA DNA template is a non-replicating vector that is an AAV-based vector derived from AAV serotype 8. The genome is a single-stranded deoxyribonucleic acid (DNA), comprising inverted terminal repeats (ITR) at each end. The ITRs flank the anti-TfR:GAA promoterless insertion template. The AAV ITRs flanking the cassette were derived from AAV2. The anti-TfR:GAA DNA template delivered by rAAV8 vector was designed as a promoterless template, thus relying on the targeted ALB locus promoter for expression.

### Example 4. Durable Alpha-Glucosidase (GAA) Expression After Insertion of Anti-CD63:GAA DNA Template in Neonatal Mice

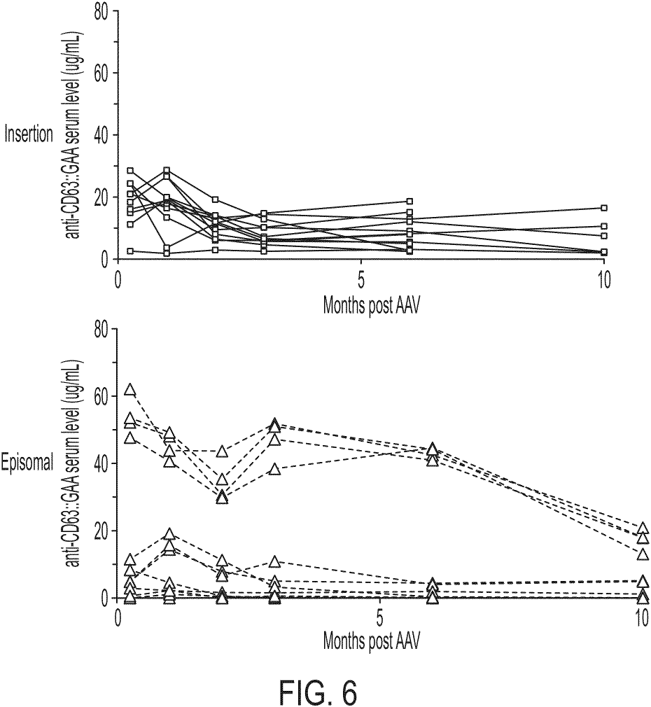

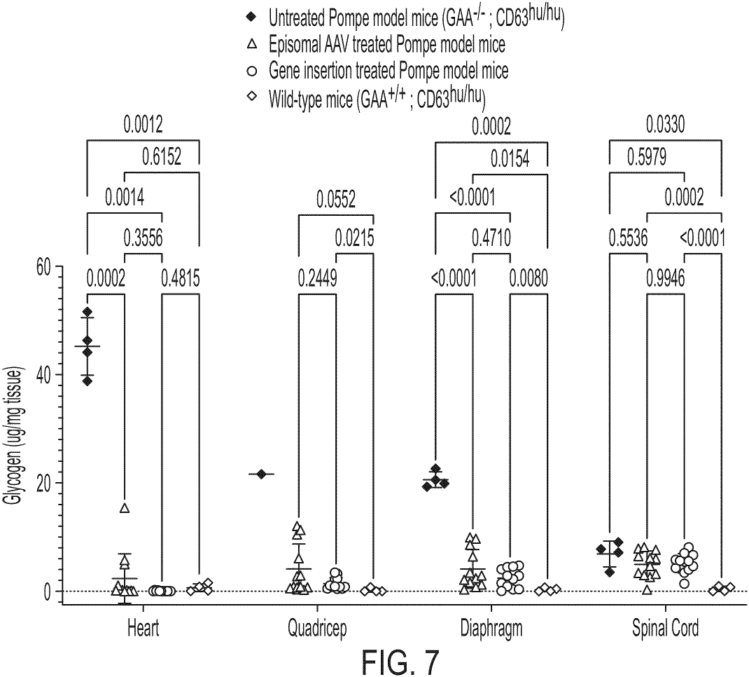

We next engineered a DNA template for insertion of a nucleic encoding anti-CD63:GAA fusions in which the C-terminus of an anti-CD63 single-chain fragment variable (scFv) is fused to the N-terminus of GAA with a glycine-serine linker (described above). We tested the anti-CD63:GAA insertion template in a Pompe disease (PD) mouse model, Gaa^{-/-} ;Cd63^{hu/hu}, where Gaa was replaced by LacZ and the protein-coding region of the Cd63 locus was replaced with its human counterpart. Adult (2-month old) male and female Gaa^{-/-};Cd63^{hu/hu} mice (62.5% C57BL/6, 37.5% 129 Sv) were dosed intravenously with the following: (1) 4e12 vg/kg recombinant AAV8 encoding anti-CD63:GAA (REGV042); or (2) 1 mg/kg LNP-g666 and 1.2e13 vg/kg recombinant AAV8 anti-CD63:GAA insertion template (REGV044). REGV042 is an episomal AAV that uses a hSerpinal enhancer and a mTTR promoter to give hepatocyte-specific expression of anti-CD63:GAA, which further includes a human albumin signal peptide. The anti-CD63:GAA coding sequences were identical in REGV042 and REGV044 and are set forth in SEQ ID NO: 194. Untreated Gaa^{-/-};Cd63^{hu/hu} mice and wild type mice were used as controls. Blood was collected and serum prepared at 7 days, 30 days, 2 months, 3 months, 6 months, and 10 months post-administration, and tissues were collected at 10 months post-administration. Anti-CD63:GAA serum levels were quantified using a plate-based sandwich ELISA that detects the scFv portion of the molecule. Anti-CD63:GAA purified protein was used as a protein standard for quantification. Data are shown in FIG. 6 and Tables 15-16. At 10 months post-administration, animals were sacrificed, and glycogen levels were quantified in muscle tissue lysates of the sacrificed animals. Tissues were dissected from mice immediately after sacrifice by CO₂ asphyxiation, snap frozen in liquid nitrogen, and stored at -80° C. Tissues were lysed on a benchtop homogenizer with stainless steel beads in distilled water for glycogen measurements or RIPA buffer for protein analyses. Glycogen analysis lysates were boiled and centrifuged to clear debris. Glycogen measurements were performed fluorometrically with a commercial kit according to manufacturer’s instructions (K646, BioVision, Milpitas, CA, USA). As shown in FIG. 7 and Tables 17-19, glycogen was significantly reduced to near wild type levels in both the episomal group and the insertion group in heart, quadricep, and diaphragm in adult mice.

TABLE 15

Serum Levels of Anti-CD63:GAA in µg/mL in Insertion Adult Group

| Months | M1 | M2 | M3 | M4 | M5 | F1 | F2 | F3 | F4 | F5 | F6 | F7 |
|---|---|---|---|---|---|---|---|---|---|---|---|---|
| 0.25 | 21.06 | 2.6 | 14.93 | 24.37 | 24.39 | 16.08 | 11.21 | 18.35 | 28.54 | 20.94 | | |
| 1 | 16.43 | 1.85 | 17.92 | 13.45 | 3.7 | 18.88 | 19.37 | 26.6 | 19.8 | 28.72 | 26.63 | 19.97 |
| 2 | 13.15 | 2.95 | 13.82 | 6.06 | 11.51 | 6.45 | 9.5 | 8.05 | 11.61 | 19.2 | 12.07 | 14.09 |
| 3 | 14.49 | 2.53 | 7.22 | 6.04 | 5.75 | 4.62 | 10.19 | 5.75 | 14.8 | 12.93 | 6.61 | 10.26 |
| 6 | 12.92 | 3.13 | 12.08 | 8.18 | 5.54 | 2.48 | 9.08 | 8 | 18.66 | 2.78 | 5.03 | 15.15 |
| 10 | 16.5 | 2 | 7.5 | 10.6 | 2.3 | | 2.4 | | | | | |

^{∗}Cells without data were due to lost samples post-collection.

TABLE 16

Serum Levels of Anti-CD63:GAA in µg/mL in Episomal Adult Group

| Months | M1 | M2 | M3 | M4 | M5 | M6 | M7 | F1 | F2 | F3 | F4 | F5 |
|---|---|---|---|---|---|---|---|---|---|---|---|---|
| 0.25 | 62.13 | 52.18 | 47.7 | 53.55 | 4.9 | 4.85 | 2.92 | 11.55 | 0 | 0 | 8.37 | 0.6 |
| 1 | 43.85 | 48.12 | 40.68 | 49.22 | 14.41 | 15.74 | 2.21 | 19.18 | 0 | 1.09 | 4.57 | 2.25 |
| 2 | 43.68 | 30.59 | 29.79 | 35.37 | 7.88 | 6.71 | 1.67 | 11.27 | 0 | 0.51 | 0.54 | 0 |
| 3 | 51.86 | 47.18 | 38.41 | 50.98 | 5.05 | 10.89 | 1.54 | 3.26 | 0 | 0 | 0.19 | 0.6 |
| 6 | 42.49 | 40.95 | 44.62 | 44.2 | 4.42 | 4.03 | 1.93 | 0.35 | 0 | 0 | 0.17 | 0.17 |
| 10 | 20.9 | 17.9 | 18 | 13.1 | 5.2 | 4.9 | 1.2 | 0.1 | 0 | 0 | 0 | 0 |

TABLE 17

Glycogen Levels in Insertion Adult Group

| | M1 | M2 | M3 | M4 | M5 | F1 | F2 | F3 | F4 | F5 | F6 | F7 |
|---|---|---|---|---|---|---|---|---|---|---|---|---|
| Heart | 0.02 | 0.02 | 0.02 | 0.01 | 0.22 | 0.02 | 0.02 | 0.19 | 0.04 | 0.02 | 0.04 | 0.02 |
| Quadricep | 0.43 | 3.24 | 0.47 | 0.79 | 2.12 | 0.5 | 2.32 | 0.82 | 1.06 | 3.45 | 0.83 | 0.89 |
| Diaphragm | 2.85 | 1.51 | 0.34 | 2.24 | 4.75 | 0.37 | 4.06 | 0.9 | 4.47 | 4.56 | 2.86 | 0.04 |
| Spinal Cord | 4.28 | 8.11 | 5.82 | 1.38 | 3.41 | 5.46 | 6.68 | 5.63 | 4 | 4.62 | 5.57 | 7.02 |

TABLE 18

Glycogen Levels in Episomal Adult Group

| | M1 | M2 | M3 | M4 | M5 | M6 | M7 | F1 | F2 | F3 | F4 | F5 |
|---|---|---|---|---|---|---|---|---|---|---|---|---|
| Heart | 0.18 | 0.13 | 0.22 | 0.02 | 0.18 | 0.02 | 0 | 1.04 | 15.39 | 4.99 | 5.74 | 0.13 |
| Quadricep | 0.87 | 0.54 | 0.72 | 0.27 | 0.71 | 0.85 | 2.78 | 6.09 | 12.05 | 10.46 | 11.31 | 2.89 |
| Diaphragm | 2.39 | 1.62 | 3.41 | 1.22 | 2.13 | 0.32 | 0.75 | 6.41 | 9.94 | 9.7 | 8.58 | 2.89 |
| Spinal Cord | 0.3 | 2.63 | 3.37 | 3.19 | 2.96 | 5.99 | 6.1 | 4.66 | 6.46 | 7.9 | 8.15 | 7.67 |

TABLE 19

Glycogen Levels in Control Adult Groups

| | Untreated Pompe mice (GAA-/- ; CD63 hu/hu) | | | | Wild-type (GAA+/+ CD63hu/hu) | | | |
|---|---|---|---|---|---|---|---|---|
| | M1 | M2 | F1 | F2 | M1 | M2 | F1 | F2 |
| Heart | 51.59 | 44.11 | 46.31 | 38.79 | 0.12 | 0.85 | 1.57 | 0.02 |
| Quadricep | 21.63 | | | | 0 | 0.74 | 0 | 0 |
| Diaphragm | 20.55 | 19.89 | 19.29 | 22.63 | 0 | 0.7 | 0.42 | 0.02 |
| Spinal Cord | 9.05 | 3.5 | 7.17 | 7.78 | 0 | 0.77 | 0.96 | 0.05 |

^{∗}Cells without data were due to experimental error.

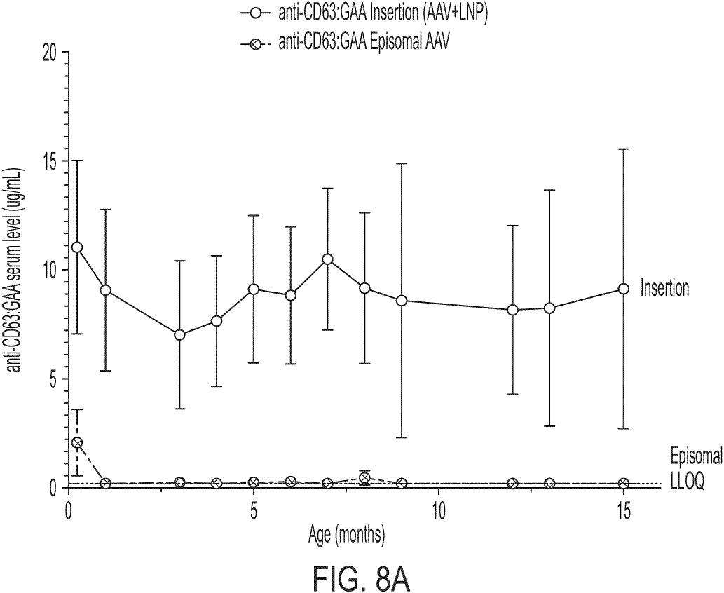

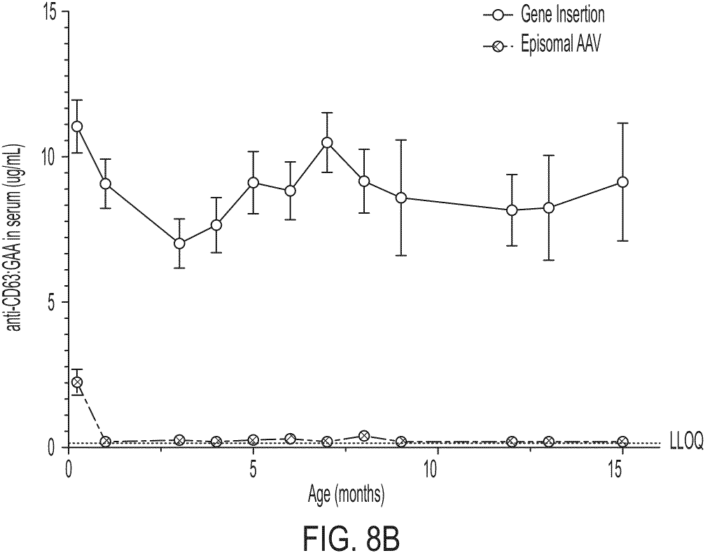

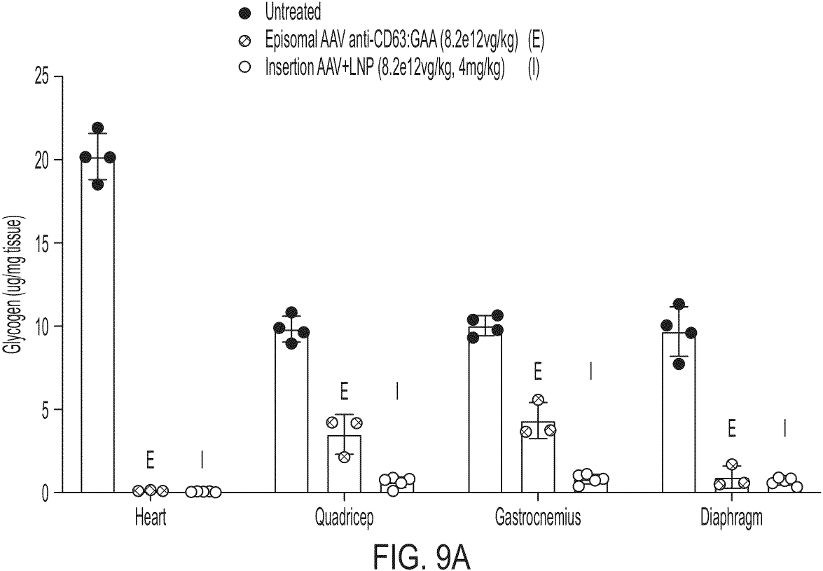

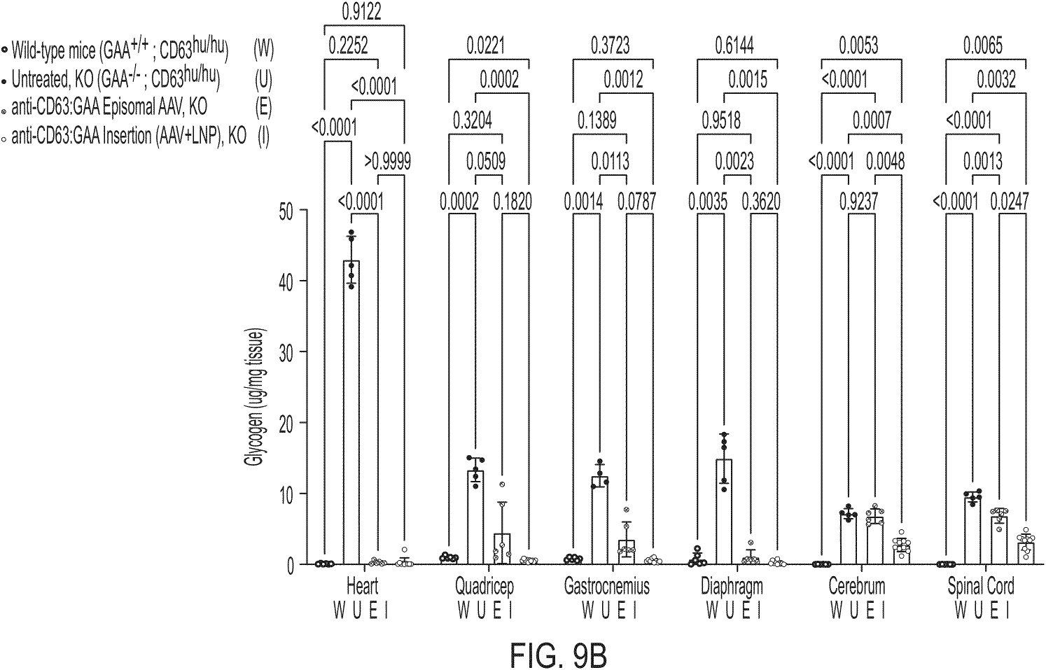

Similar experiments were then performed in which neonatal Gaa^{-/-};Cd63^{hu/hu} mice (62.5% C57BL/6, 37.5% 129 Sv) were dosed intravenously at P1 with the following: (1) 8.2e12 vg/kg recombinant AAV8 encoding anti-CD63:GAA (REGV042); or (2) 4 mg/kg LNP-g666, and 8.2e12 vg/kg recombinant AAV8 anti-CD63:GAA insertion template (REGV044). Untreated Gaa^{-/-};Cd63^{hu/hu} mice and wild type mice were used as controls. Blood was collected and serum prepared at 7 days, 30 days, 2 months, 3 months, 4 months, 5 months, 6 months, 7 months, 8 months, 9 months, 12 months, 13 months, and 15 months, and tissues were collected at 3 months and 15 months post-administration. As shown in FIGS. 8A-8B and Tables 20-21, in contrast to what was observed when adult mice were dosed, the serum anti-CD63:GAA levels were stable over the 15-month time course in the insertion group, but the episomal group started out lower and dropped off to below the lower limit of quantification using the serum ELISA assay within 1 month when neonatal mice were dosed. Similarly, as shown in FIG. 9A and Table 22, glycogen storage at 3 months was normalized to wild type levels in heart, quadricep, gastrocnemius, and diaphragm in the insertion group, but not in the episomal group. Likewise, as shown in FIG. 9B and Table 23, glycogen storage at 15 months was normalized to wild type levels in heart, quadricep, gastrocnemius, and diaphragm in the insertion group, and glycogen storage was partially corrected in CNS tissues in the insertion group but not the episomal group.

TABLE 20

Serum Anti-CD63:GAA Levels (µg/mL) in Neonatal Mice with Insertion Group

| Months | Insertion (AAV+LNP) | | | | | | | | | | | | | | | | | | |
|---|---|---|---|---|---|---|---|---|---|---|---|---|---|---|---|---|---|---|---|
| | ^{∗} | ^{∗} | ^{∗∗} | ^{∗∗} | ^{∗∗} | | | | | | | | ^{∗∗} | | | | ^{∗} | ^{∗} | ^{∗} |
| 0.23 | 18.3 | 14.4 | 15.9 | 12.2 | 12.8 | 8.7 | 5.9 | 15.1 | 9.5 | 14.6 | 8.2 | 11.6 | 5.7 | 8.4 | 14 | 13.7 | 8.8 | 8.6 | 3.4 |
| 1 | 13.2 | 14.8 | 13.9 | 9 | 10.3 | 5.4 | 3.9 | 11.1 | 6.1 | 11.2 | 8 | 9.9 | 4.7 | 9.4 | 11.5 | 8.7 | 10.2 | 11.1 | 0 |
| 3 | 10.2 | 10.9 | | | | 3.5 | 2.3 | 10.3 | 5.8 | 7.2 | 6.9 | 8.2 | 2.7 | 7.9 | 14.7 | 5.5 | 7.2 | 6.2 | 2.8 |
| 4 | | | | | | 5.2 | 2.8 | 8.7 | 7.2 | 8.7 | 6.8 | 8.4 | | 7.4 | 14.5 | 6.8 | | | |
| 5 | | | | | | 7.1 | 4.1 | 12.1 | 6.6 | 10.2 | 8.1 | 9 | | 8.5 | 16.6 | 8.8 | | | |
| 6 | | | | | | 6.9 | 4.2 | 14.8 | 7 | 10.2 | 7 | 7.9 | | 9.2 | 13.1 | 8 | | | |
| 7 | | | | | | 8.9 | 4.9 | 17.5 | 11.2 | 10.4 | 9.9 | 10 | | 9.1 | 13.4 | 9.6 | | | |
| 8 | | | | | | 8.5 | 4.4 | 17 | 6 | 8.9 | 8.4 | 8.5 | | 8.1 | 12.5 | 9.3 | | | |
| 9 | | | | | | 5.9 | 3.1 | 10.7 | 5.2 | 25.4 | 7.5 | 5.9 | | 6.5 | 9.4 | 6.2 | | | |
| 12 | | | | | | 6.7 | 3.6 | 10.3 | 6.8 | 17.8 | 6.8 | 7.3 | | 6.7 | 9.7 | 5.9 | | | |
| 13 | | | | | | 4.6 | 3.7 | 17.4 | 4.2 | 17.0 | N/A | 6.1 | | 5.3 | 10.3 | 5.6 | | | |
| 15 | | | | | | 5.1 | 3.8 | 18.4 | 4.0 | 18.3 | 7.6 | 3.9 | | 5.5 | 18.0 | 6.6 | | | |

^{∗}Mouse sacrificed at 3 months for 3-month glycogen assay
^{∗∗}Mouse died

TABLE 21

Serum Anti-CD63:GAA Levels (µg/mL) in Neonatal Mice with Episomal Group

| Months | Episomal AAV | | | | | | | | | | |
|---|---|---|---|---|---|---|---|---|---|---|---|
| | ^{∗} | ^{∗} | ^{∗} | ^{∗∗} | | | ^{∗∗} | | | | |
| 0.23 | 0 | 0 | 1.9 | 0 | 2.2 | 4.1 | 2.5 | 2.7 | 3.7 | 3.4 | 3.6 |
| 1 | 0 | 0 | 0 | 0 | 0 | 0 | 0 | 0 | 0 | 0 | 0 |
| 3 | 0 | 0 | 0 | 0 | 0 | 0 | 0 | 0.3 | 0.4 | 0.5 | 0 |
| 4 | | | | 0 | 0 | 0 | 0 | 0 | 0 | 0 | 0 |
| 5 | | | | | 0 | 0 | 0 | 0 | 0 | 0.6 | 0 |
| 6 | | | | | 0 | 0 | 0 | 0 | 0.5 | 0.6 | 0 |
| 7 | | | | | 0 | 0 | 0 | 0 | 0 | 0 | 0 |
| 8 | | | | | 0 | 0.5 | 0 | 0 | 0.5 | 1 | 0 |
| 9 | | | | | 0 | 0 | 0 | 0 | 0 | 0 | 0 |
| 12 | | | | | 0 | 0 | 0 | 0 | 0 | 0 | 0 |
| 13 | | | | | 0 | 0 | 0 | 0 | 0 | 0 | 0 |
| 15 | | | | | 0 | 0 | | 0 | 0 | 0 | 0 |

^{∗}Mouse sacrificed

TABLE 22

Glycogen Levels (µg/mg Tissue) in Neonatal Mice

| | Untreated KO | | | | Episomal AAV | | | Gene Insertion | | | | |
|---|---|---|---|---|---|---|---|---|---|---|---|---|
| Heart | 20.16 | 18.52 | 20.14 | 21.92 | 0.16 | 0.1 | 0.1 | 0.08 | 0.06 | 0.08 | 0.04 | 0.02 |
| Quadricep | 8.96 | 10.83 | 9.89 | 9.63 | 4.18 | 2.13 | 4.21 | 0.89 | 0.77 | 0.82 | 0.1 | 0.58 |
| Gastrocnemius | 10.39 | 10.65 | 9.31 | 9.77 | 5.58 | 3.66 | 3.75 | 1.04 | 0.83 | 1.12 | 0.38 | 0.73 |
| Diaphragm | 10.05 | 7.73 | 11.33 | 9.6 | 0.5 | 0.59 | 1.7 | 0.57 | 0.7 | 0.85 | 0.34 | 0.9 |

TABLE 23

Glycogen Levels (µg/mg Tissue) in Neonatal Mice
WT

| | | | | | | |
|---|---|---|---|---|---|---|
| Heart | 0.18 | 0.03 | 0.09 | 0.12 | 0.04 | 0.04 |
| Quadricep | 0.95 | 0.77 | 0.86 | 1.01 | 1.35 | 0.85 |
| Gastrocnemius | 0.8 | 0.68 | 0.61 | 1.02 | 1.06 | 0.53 |
| Diaphragm | 0.6 | 0.12 | 2.24 | 1.25 | 0.22 | 0.03 |
| Cerebrum | 0.02664 | 0 | 0 | 0.04436 | 0 | 0.00255 |
| Spinal Cord | 0.01999 | 0 | 0 | 0 | 0.026 | 0 |

Untreated

| Heart | 40.73 | 42.15 | 45.87 | 46.84 | 39.15 | | | | | |
|---|---|---|---|---|---|---|---|---|---|---|
| Quadricep | 12.42 | 11.02 | 14.71 | 15.05 | 13.43 | | | | | |
| Gastrocnemius | ^{∗} | 10.99 | 11.6 | 14.56 | 12.86 | | | | | |
| Diaphragm | 16.49 | 10.57 | 18.33 | 17.3 | 11.87 | | | | | |
| Cerebrum | 7.28 | 6.26 | 7.02 | 6.98 | 8.23 | | | | | |
| Spinal Cord | 8.46 | 10.34 | 9.37 | 9.94 | 9.49 | | | | | |
| | P1 episomal | | | | | | | | | |
| Heart | 0.42 | 0.17 | 0.69 | 0.21 | 0.09 | 0.09 | | | | |
| Quadricep | 11.27 | 8.47 | 1.85 | 2.72 | 1.43 | 0.96 | | | | |
| Gastrocnemius | 7.73 | 5.36 | 2.02 | 2.18 | 2.03 | 1.84 | | | | |
| Diaphragm | 3.09 | 0.8 | 0.74 | 0.76 | 0.33 | 0.42 | | | | |
| Cerebrum | 8.26 | 5.63 | 7.25 | 7.48 | 5.87 | 6.33 | | | | |
| Spinal Cord | 6.42 | 4.93 | 7.46 | 7.13 | 7.61 | 7.55 | | | | |
| | P1 insertion | | | | | | | | | |
| Heart | 0.04 | 0.12 | 0.13 | 0.06 | 0.25 | 2.1 | 0 | 0.05 | 0.04 | 0.07 |
| Quadricep | 0.6 | 0.64 | 0.68 | 0.54 | 0.58 | 0.61 | 0.27 | 0.85 | 0.35 | 0.5 |
| Gastrocnemius | 0.43 | 0.65 | 0.51 | 0.51 | 0.46 | 0.9 | 0.27 | 1.06 | 0.24 | 0.43 |
| Diaphragm | 0.16 | 0.29 | 0.08 | 0.12 | 0.39 | 0.77 | 0 | 0 | 0.59 | 0.12 |
| Cerebrum | 3.09 | 4.59 | 1.18 | 3.06 | 1.87 | 2.42 | 3.33 | 2.2 | 2.39 | 3.29 |
| Spinal Cord | 3.61 | 4.89 | 1.88 | 3.55 | 1.03 | 3.79 | 3.39 | 3.72 | 2.23 | 3.42 |

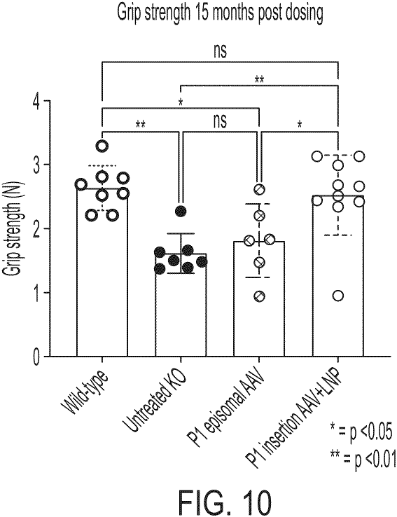

To assess whether the improved glycogen reduction observed with the insertion template in neonatal mice translated into improved muscle function, the mice were tested on grip strength apparatuses at 15 months post-administration. Limb grip strength was measured with a force meter (Columbus Instruments, Columbus, OH, USA). All tests were performed in triplicate. Mice treated with the insertion template showed significantly improved performance compared to mice treated with the episomal construct on the grip strength test. In fact, the grip strength in the insertion group tracked closely with that of wild type mice at 15 months posttreatment, whereas there was no difference in the grip strength in the episomal group tracked compared to the untreated group. See FIG. 10 and Table 24. These results show that, in neonatal mice, the insertion approach shows vastly improved durability of expression compared to the episomal approach, and better substrate reduction, indicating that insertion is the superior approach for pediatric indications.

TABLE 24

Grip Strength (Newtons) in Neonatal Mice

| Wild-type | Untreated KO | P1 insertion AAV+LNP | P1 episomal AAV |
|---|---|---|---|
| 2.21 | 1.39 | 2.44 | 1.83 |
| 2.21 | 1.63 | 2.34 | 1.47 |
| 2.52 | 1.48 | 2.51 | 0.94 |
| 2.55 | 1.66 | 2.68 | 1.81 |
| 2.81 | 1.37 | 3.13 | 2.2 |
| 2.69 | 2.27 | 0.95 | 2.61 |
| 3.29 | 1.5 | 2.99 | |
| 2.79 | | 2.42 | |
| | | 2.66 | |
| | | 3.13 | |

An experiment was performed in which 4-month old Gaa^{-/-};Alb^{hu/hu} mice (n=3) were dosed intravenously with 7.5e10 vg/mouse recombinant AAV8 anti-CD63:GAA insertion template (REGV044) and 1 mg/kg LNP-g9860 in order to validate that anti-CD63:GAA can be inserted into mice humanized for albumin using human albumin gRNA. Blood was collected and serum prepared at 7 days, 14 days, 35 days, and 60 days post-administration. GAA serum levels up to ~3 µg/mL were observed and were maintained over the time course (data not shown), confirming that anti-CD63:GAA can be inserted into mice humanized for albumin using human albumin gRNA.

In summary, the combination of the highly precise and targeted CRISPR/Cas9 technology delivered by LNP and the anti-CD63:GAA DNA template delivered by the selected rAAV8 vector allows for long-term expression of anti-CD63:GAA protein from hepatocytes and delivery to muscle cells affected in PD, potentially providing a life-long effective treatment to PD patients, including neonatal patients.

These results show that, in neonatal mice, the insertion approach shows vastly improved durability of expression compared to the episomal approach, indicating that insertion is the superior approach in neonatal subjects.

### Example 5. Optimized Anti-CD63:GAA DNA Templates

Optimized anti-CD63:GAA templates were generated to develop a lead for non-human primate (NHP) studies. To select a development candidate, several versions of the insertion cassette were generated in which the nucleotide sequence encoding the anti-CD63:GAA was modified (e.g., by depleting CpGs). Table 25 lists the different versions of anti-CD63:GAA inserts designed. In addition, the GS 50 construct was further optimized to remove the six activating CpG motifs but leaving the 44 non-activating CpG motifs (GS 44; SEQ ID NO: 200). The GS 0 construct was further optimized (GS 0v2; SEQ ID NO: 198) by removing an AATAA polyA, removing a cryptic splice acceptor, and replacing a glycine encoding GGGGGGGGGGG (SEQ ID NO: 203) with GGAGGAGGTGG (SEQ ID NO: 204).

TABLE 25

Anti-CD63:GAA Inserts for Insertion Cassettes

| Anti-CD63:GAA Insert | Transgene CpG Content | SEQ ID NO |
|---|---|---|
| First generation | 181 | 194 |
| GS 0 | 0 | 197 |
| GS 0v2 | 0 | 198 |
| DC 0 | 0 | 195 |
| GS 1 | 1 | 199 |
| RE 8 | 8 | 202 |
| GS 50 | 50 | 201 |
| GS 44 | 44 | 200 |
| GA 0 | 0 | 196 |

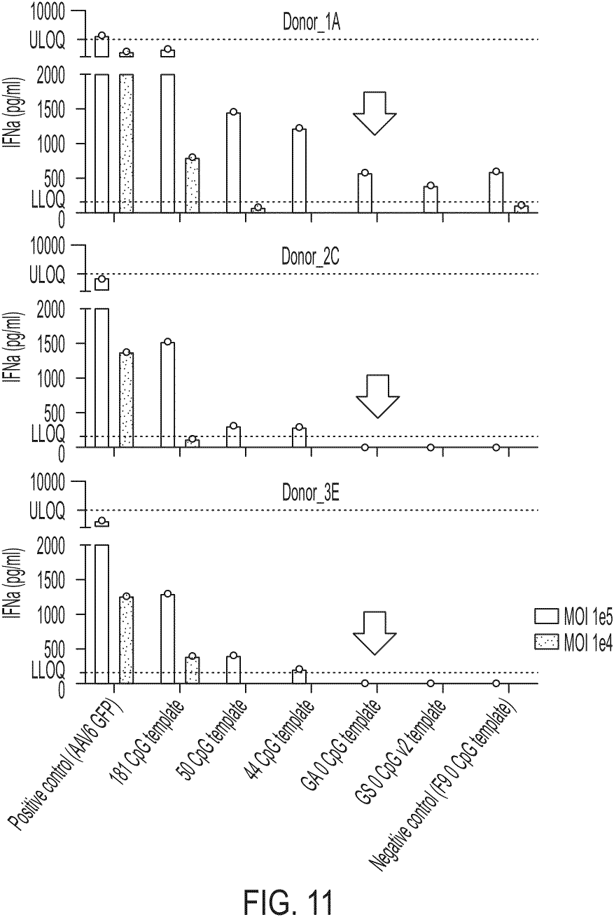

Peripheral blood mononuclear cells (PBMCs) were isolated from human blood. Plasmacytoid dendritic cells (pDCs) were enriched and combined with pBMCs (1e4 pDCs + 1e5 PBMCs per well). The cells were incubated for 16-18 hours with AAV6 or control CpG-oligodeoxynucleotides (ODNs). The supernatants were harvested, and an IFNα ELISA was performed. This assay assessed whether CpG-depleted anti-CD63:GAA sequences exhibited reduced IFN-I responses in a primary human plasmacytoid DC-based assay as compared to non-CpG-depleted sequences. The tested samples, including a positive control AAV6-GFP and a negative control CpG-depleted (0 CpG F9 transgene) template, are shown in Table 26. The results are shown in FIG. 11.

TABLE 26

AAV6 Anti-CD63:GAA Templates Tested for IFNα response in pDC/AAV assay.

| Genome | Transgene CpG content | TOTAL CpG content w/ ITRs |
|---|---|---|
| AAV6 GFP (SFFV eGFP WPRE) | 99 | 175 |
| First generation | 181 | 213 |
| GS 50 | 50 | 82 |
| GS 44 | 44 | 75 |
| GA 0 | 0 | 32 |
| GS 0 v2 | 0 | 32 |
| F9 0 CpG template | 0 | 33 |

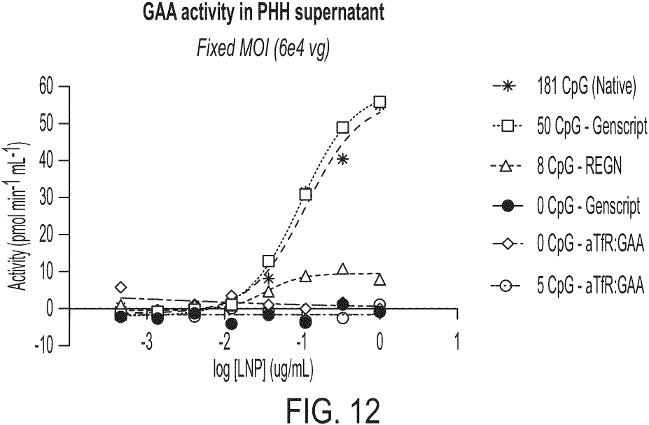

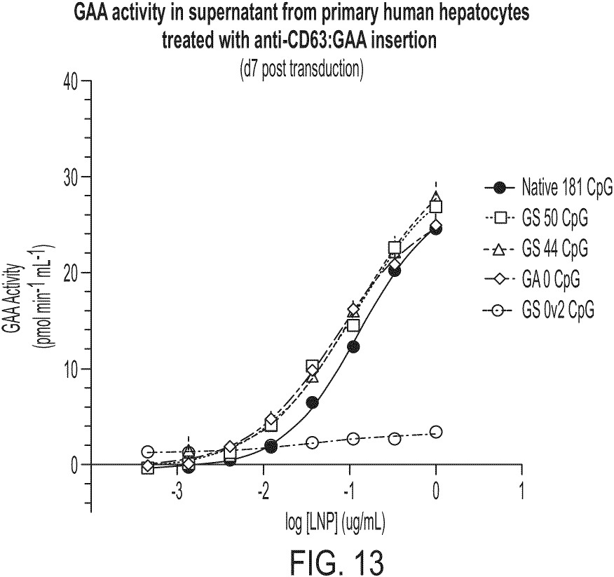

Activity of the optimized templates was tested in a primary human hepatocyte assay. AAV templates were packaged into AAV2 viruses. Primary human hepatocytes were grown in 96-well plates and administered the AAV (AAV2, AAV6, or AAV8) containing the template DNA and LNP-g9860 at either fixed MOI with LNP dose titration or fixed LNP concentration with AAV dose titration. Supernatants were collected 7 days post-dosing and stored at -80° C. Supernatants were thawed and GAA activity in the supernatants was measured using a 4-methylumbelliferone-based fluorometric assay (K690, BioVision, Milpitas, CA, USA) as a measurement of amount of enzymatically active GAA produced and secreted from the cells. A first batch of optimized anti-CD63:GAA and anti-TfR:GAA templates were tested using AAV2 as shown in Table 27 and FIG. 12. The 50 CpG anti-CD63:GAA template expressed similarly to the native template, but the initial templates with fewer CpGs did not express well. Additional optimized anti-CD63:GAA templates were tested using AAV6 as shown in Table 28 and FIG. 13. The GA 0 CpG anti-CD63:GAA template maintained expression compared to the native high CpG template in the in vitro primary human hepatocyte insertion assay. Similar results were observed using AAV8 instead of AAV6 (data not shown). The GA 0 CpG anti-CD63:GAA template produced functional GAA in the primary human hepatocyte assay at levels similar to the native while also producing reduced IFNα responses in the pDC/AAV assay.

TABLE 27

AAV2 Anti-CD63:GAA and Anti-TfR:GAA Templates Tested for GAA Activity in PHH Supernatant

| Genome | Transgene CpG content |
|---|---|
| First generation | 181 |
| GS 50 | 50 |
| RE 8 | 8 |
| GS 0 | 0 |
| 12837scfv-GAA-1 0CpG 27 | 0 |
| 12837scfv-GAA-2 5CpG 27 | 5 |

TABLE 28

AAV6 Anti-CD63:GAA Templates Tested for GAA Activity in PHH Supernatant

| Genome | Transgene CpG content |
|---|---|
| First generation | 181 |
| GS 50 | 50 |
| GS 44 | 8 |
| GA 0 | 0 |
| GS 0v2 | 0 |

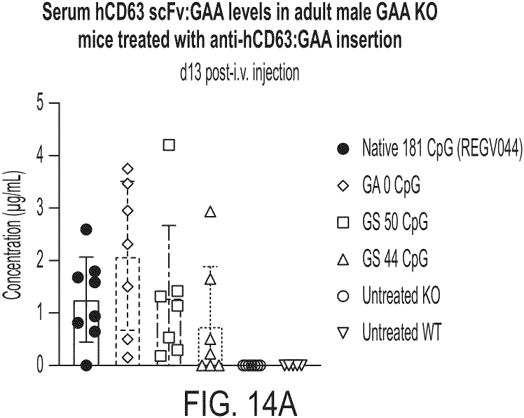

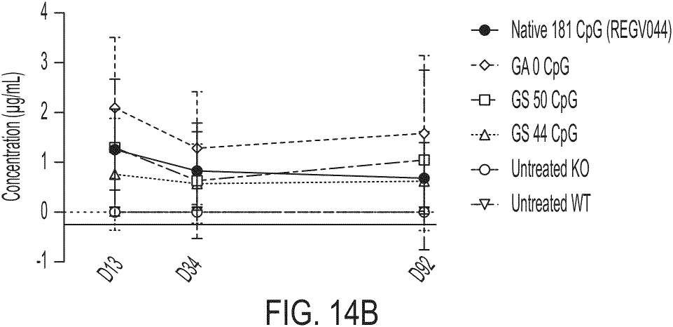

Expression of the optimized templates was validated following insertion in Gaa^{-/-} mice as described in Example 4. Adult mice were dosed intravenously at with 1.97e12 vg/kg recombinant AAV8 anti-CD63:GAA insertion template (REGV044) and 1 mg/kg LNP-g666. The recombinant AAV8 templates tested included the native 181 CpG anti-CD63:GAA insertion template (REGV044), the GA 0 CpG template, the GSa50 CpG template, and the GS 44 CpG template. Untreated Gaa^{-/-} mice and wild type mice were used as controls. Blood was collected and serum prepared at 13 days, 34 days, and 92 days post-administration. As shown in FIG. 14A, the GA 0 CpG template showed similar serum expression in vivo in adult Gaa^{-/-} mice compared to the native high CpG template. As shown in FIG. 14B, the serum expression levels were consistent over three time points.

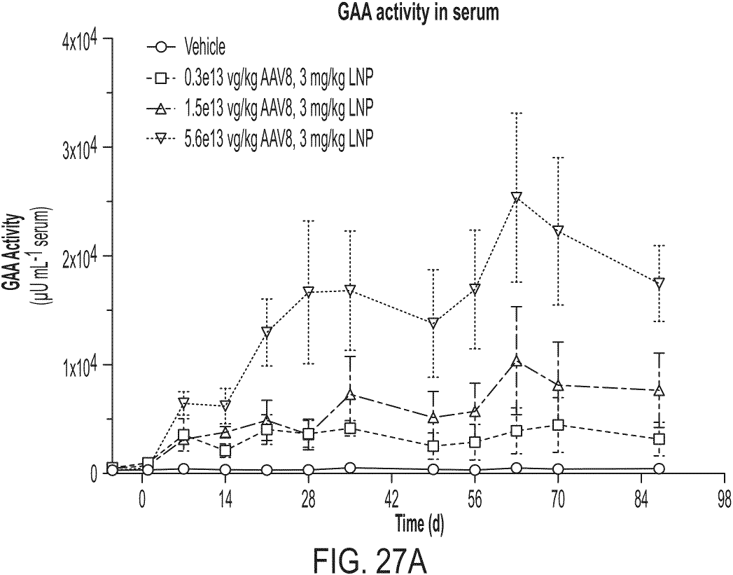

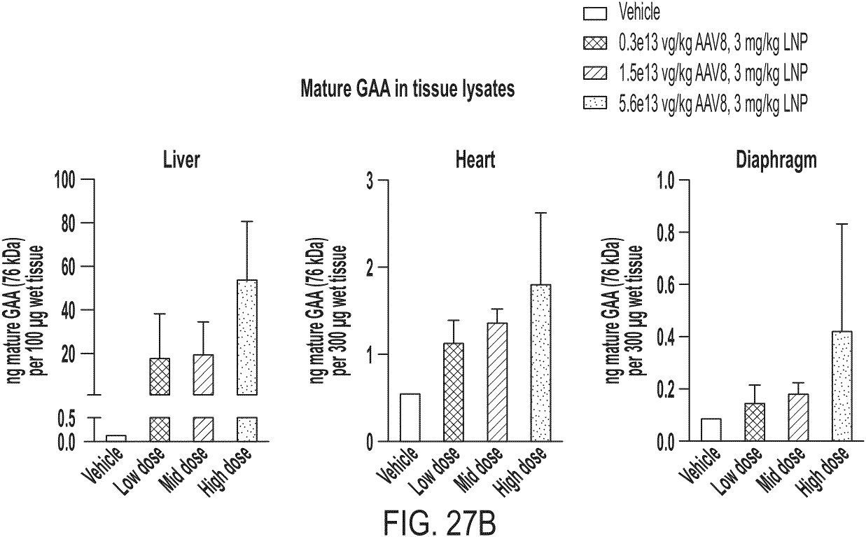

Expression of the GA 0 CpG anti-CD63:GAA template (SEQ ID NOS: 196 and 736) was evaluated following insertion in non-human primates. Two-year old cynomolgus macaques were administered recombinant AAV8 containing the CpG depleted anti-CD63:GAA template and an LNP-g9860 targeting the cynomolgus albumin intron 1. Three different recombinant AAV8 doses were used (0.3e13vg/kg, 1.5e13vg/kg, and 5.6e13vg/kg) with a 3 mg/kg LNP dose. N=1 in the vehicle control group, and N=3 in the dosed groups. Serum GAA activity was measured using a fluorometric substrate assay in the monkeys through the course of the study. An AAV-dose-dependent increase in serum GAA activity levels was observed over time in the groups (FIG. 27A). This indicated successful insertion of the anti-CD63:GAA transgene into the albumin locus and resultant expression and secretion of the transgene in the animals. Tissues were collected at sacrifice (Day 89) and probed by western blot for presence of a 76 kDa lysosomal form of GAA. A dose-dependent increase of lysosomal GAA in the tissues was observed in the heart and diaphragm (FIG. 27B), which indicated delivery of the liver-derived anti-CD63:GAA protein to the distal tissues.

Activity of the optimized templates is validated in the PD mouse model, Gaa^{-/-} ;Cd63^{hu/hu}, as described in Example 4.

Expression of the optimized templates is evaluated in non-human primates. Two LNP doses and two gRNAs are tested. Specifically, expression is evaluated by administering 1 or 3 mg/kg of LNP-g9860 as described in Example 1 and 1.5e13 vg/kg of rAAV8 comprising each optimized template. An LNP with gRNA9844 is also used. Expression is analyzed over a 12-week study. Tissues are also collected for analysis of biodistribution of GAA, and GAA activity is assessed in collected tissues.

### Example 6. Durable Alpha-Glucosidase (GAA) Expression After Insertion of Anti-TfR:GAA DNA Template in Neonatal Mice

Anti-human transferrin receptor (hTfR) antibodies were generated and screened for the ability to bind hTfR and for lack of strong blocking of human transferrin-hTfR binding. Based on this initial analysis, 32 variable sequences were chosen. See Table 29.

TABLE 29

Domains in Anti-hTfR Antibodies, Antigen-binding Fragments (e.g., Fabs) or scFv Molecules in Fusion Proteins

| # | anti-hTfR Molecule | HC-VR NT | HC-VR AA | HCDR1 | HCDR2 | HCDR3 | LC-VR NT | LC-VR AA | LCDR1 | LCDR2 | LCDR3 |
|---|---|---|---|---|---|---|---|---|---|---|---|
| 1 | 31874B | 216 | 217 | 218 | 219 | 220 | 221 | 222 | 223 | 224 | 225 |
| 2 | 31863B | 226 | 227 | 228 | 229 | 230 | 231 | 232 | 233 | 234 | 235 |
| 3 | 69348 | 236 | 237 | 238 | 239 | 240 | 241 | 242 | 243 | 244 | 245 |
| 4 | 69340 | 246 | 247 | 248 | 249 | 250 | 251 | 252 | 253 | 254 | 255 |
| 5 | 69331 | 256 | 257 | 258 | 259 | 260 | 261 | 262 | 263 | 264 | 265 |
| 6 | 69332 | 266 | 267 | 268 | 269 | 270 | 271 | 272 | 273 | 274 | 275 |
| 7 | 69326 | 276 | 277 | 278 | 279 | 280 | 281 | 282 | 283 | 284 | 285 |
| 8 | 69329 | 286 | 287 | 288 | 289 | 290 | 291 | 292 | 293 | 294 | 295 |
| 9 | 69323 | 296 | 297 | 298 | 299 | 300 | 301 | 302 | 303 | 304 | 305 |
| 10 | 69305 | 306 | 307 | 308 | 309 | 310 | 311 | 312 | 313 | 314 | 315 |
| 11 | 69307 | 316 | 317 | 318 | 319 | 320 | 321 | 322 | 323 | 324 | 325 |
| 12 | 12795B | 326 | 327 | 328 | 329 | 330 | 331 | 332 | 333 | 334 | 335 |
| 13 | 12798B | 336 | 337 | 338 | 339 | 340 | 341 | 342 | 343 | 344 | 345 |
| 14 | 12799B | 346 | 347 | 348 | 349 | 350 | 351 | 352 | 353 | 354 | 355 |
| 15 | 12801B | 356 | 357 | 358 | 359 | 360 | 361 | 362 | 363 | 364 | 365 |
| 16 | 12802B | 366 | 367 | 368 | 369 | 370 | 371 | 372 | 373 | 374 | 375 |
| 17 | 12808B | 376 | 377 | 378 | 379 | 380 | 381 | 382 | 383 | 384 | 385 |
| 18 | 12812B | 386 | 387 | 388 | 389 | 390 | 391 | 392 | 393 | 394 | 395 |
| 19 | 12816B | 396 | 397 | 398 | 399 | 400 | 401 | 402 | 403 | 404 | 405 |
| 20 | 12833B | 406 | 407 | 408 | 409 | 410 | 411 | 412 | 413 | 414 | 415 |
| 21 | 12834B | 416 | 417 | 418 | 419 | 420 | 421 | 422 | 423 | 424 | 425 |
| 22 | 12835B | 426 | 427 | 428 | 429 | 430 | 431 | 432 | 433 | 434 | 435 |
| 23 | 12847B | 436 | 437 | 438 | 439 | 440 | 441 | 442 | 443 | 444 | 445 |
| 24 | 12848B | 446 | 447 | 448 | 449 | 450 | 451 | 452 | 453 | 454 | 455 |
| 25 | 12843B | 456 | 457 | 458 | 459 | 460 | 461 | 462 | 463 | 464 | 465 |
| 26 | 12844B | 466 | 467 | 468 | 469 | 470 | 471 | 472 | 473 | 474 | 475 |
| 27 | 12845B | 476 | 477 | 478 | 479 | 480 | 481 | 482 | 483 | 484 | 485 |
| 28 | 12839B | 486 | 487 | 488 | 489 | 490 | 491 | 492 | 493 | 494 | 495 |
| 29 | 12841B | 496 | 497 | 498 | 499 | 500 | 501 | 502 | 503 | 504 | 505 |
| 30 | 12850B | 506 | 507 | 508 | 509 | 510 | 511 | 512 | 513 | 514 | 515 |
| 31 | 69261 | 516 | 517 | 518 | 519 | 520 | 521 | 522 | 523 | 524 | 525 |
| 32 | 69263 | 526 | 527 | 528 | 529 | 530 | 531 | 532 | 533 | 534 | 535 |

### 31874B

HCVR (V_{H}) Nucleotide Sequence

GAGGTGCAGCTGGTGGAGTCTGGGGGAGGCTTGGTACAGCCTGGGGGGTC
CCTGAGACTCTCCTGTGCAGCCTCTGGATTCGCCTTTAGCAGCTATGCCA
TGACCTGGGTCCGACAGGCTCCAGGGAAGGGGCTGGAGTGGGTCTCAGTT
ATCAGTGGTACTGGTGGTAGTACATACTACGCAGACTCCGTGAAGGGCCG
GTTCACCATCTCCAGAGACAATTCCAAGAACACGCTGTATCTACAAATGA
ACAGCCTGAGAGCCGAGGACACGGCCGTATATTACTGTGCGAAAGGGGGA
GCAGCTCGTAGAATGGAATACTTCCAGTACTGGGGCCAGGGCACCCTGGT
CACCGTCTCCTCA (SEQ ID NO: 216)

HCVR (V_{H}) Amino Acid Sequence

EVQLVESGGGLVQPGGSLRLSCAASGFAFSSYAMTWVRQAPGKGLEWVSV
ISGTGGSTYYADSVKGRFTISRDNSKNTLYLQMNSLRAEDTAVYYCAKGG
AARRMEYFQYWGQGTLVTVSS (SEQ ID NO: 217)

HCDR1:GFAFSSYA (SEQ ID NO:218)

HCDR2:ISGTGGST (SEQ ID NO: 219)

HCDR3: AKGGAARRMEYFQY (SEQ ID NO: 220)

LCVR (V_{L}) Nucleotide Sequence

GACATCCAGATGACCCAGTCTCCATCCTCCCTGTCTGCATCTGTAGGAGA
CAGAGTCACCATCACTTGCCGGGCGAGTCAGGGCATTAGCAATTATTTAG
CCTGGTATCAGCAGAAACCAGGGAAAGTTCCTAACCTCCTTATCTATGCT
GCATCCACTTTGCAATCAGGGGTCCCATCTCGATTCAGTGGCAGTGGATC
TGGGACAGATTTCACTCTCACCATCAGCAGCCTGCAGCCTGAAGATGTTG
CAACTTATTACTGTCAAAAGTATAACAGTGCCCCTCTCACTTTCGGCGGA
GGGACCAAGGTGGAGATCAAA (SEQ ID NO: 221)

LCVR (V_{L}) Amino Acid Sequence

DIQMTQSPSSLSASVGDRVTITCRASQGISNYLAWYQQKPGKVPNLLIYA
ASTLQSGVPSRFSGSGSGTDFTLTISSLQPEDVATYYCQKYNSAPLTFGG
GTKVEIK (SEQ ID NO: 222)

LCDR1: QGISNY (SEQ ID NO: 223)

LCDR2: AAS (SEQ ID NO: 224)

LCDR3: QKYNSAPLT (SEQ ID NO: 225)

### 31863B

HCVR (V_{H}) Nucleotide Sequence

GAGGTGCAGCTGGTGGAGTCTGGGGGAGGCTTGGTACAGCCTGGGGGGTC
CCTGAGACTCTCCTGTGCAGCCTCTGGATTCACCTTTAACAGCTATGCCA
TGACCTGGGTCCGCCAGGCTCCAGGGAAGGGGCTGGAGTGGGTCTCATTT
ATTGGTGGTAGTACTGGTAACACATACTACGCAGGCTCCGTGAAGGGCCG
GTTCACCATCTCCAGCGACAATTCCAAGAAGACGCTGTATCTGCAAATGA
ACAGCCTGAGAGCCGAGGACACGGCCGTATATTACTGTGCGAAAGGGGGA
GCAGCTCGTAGAATGGAATACTTCCAGCACTGGGGCCAGGGCACCCTGGT
CACCGTCTCCTCA (SEQ ID NO: 226)

HCVR (V_{H}) Amino Acid Sequence

EVQLVESGGGLVQPGGSLRLSCAASGFTFNSYAMTWVRQAPGKGLEWVSF
IGGSTGNTYYAGSVKGRFTISSDNSKKTLYLQMNSLRAEDTAVYYCAKGG
AARRMEYFQHWGQGTLVTVSS (SEQ ID NO: 227)

HCDR1: GFTFNSYA (SEQ ID NO: 228)

HCDR2:IGGSTGNT (SEQ ID NO: 229)

HCDR3:AKGGAARRMEYFQH (SEQ ID NO: 230)

LCVR (V_{L}) Nucleotide Sequence

GACATCCAGATGACCCAGTCTCCATCCTCCCTGTCTGCATCTATAGGAGA
CAGAGTCACCATCACTTGCCGGGCGAGTCAGGGCATTAGCAATTATTTAG
CCTGGTATCAACAGAAACCAGGGAAAGTTCCTAAGCTCCTGATCTATGCT
GCATCCACTTTGCAATCAGGGGTCCCATCTCGGTTCAGTGGCAGTGGATC
TGGGACAGATTTCACTCTCACCATCAGCAGCCTGCAGCCTGAAGATGTTG
CAACTTATTACTGTCAAAACCATAACAGTGTCCCTCTCACTTTCGGCGGA
GGGACCAAGGTGGAGATCAAA (SEQ ID NO: 231)

LCVR (V_{L}) Amino Acid Sequence

DIQMTQSPSSLSASIGDRVTITCRASQGISNYLAWYQQKPGKVPKLLIYA
ASTLQSGVPSRFSGSGSGTDFTLTISSLQPEDVATYYCQNHNSVPLTFGG
GTKVEIK (SEQ ID NO: 232)

LCDR1: QGISNY (SEQ ID NO: 233)

LCDR2: AAS (SEQ ID NO: 234)

LCDR3: QNHNSVPLT (SEQ ID NO: 235)

### 69348

HCVR (V_{H}) Nucleotide Sequence

CAGGTGCAGCTGGTGGAGTCTGGGGGAGGCGTGGTCCAGCCTGGGAGGTC
CCTGAGACTCTCCTGTGCAGCGTCTGGATTCACCTTCACTACCTATGGCA
TGCACTGGGTCCGCCAGGCTCCAGGCAAGGGGCTGGAGTGGGTGGCTGTT
ATATGGTATGATGGAAGTAATAAATATTATGGAGACTCCGTGAAGGGCCG
ATTCACCATCTCCAGAGACAATTCCAAGAACACACTGTATCTGCAAATGA
ACAGCCTGAGAGTCGACGACACGGCTGTTTATTACTGTACGAGAACCCAT
GGCTATACCAGGTCGTCGGACGGTTTTGACTACTGGGGCCAGGGAACCCT
GGTCACCGTCTCCTCA(SEQ ID NO: 236)

HCVR (V_{H}) Amino Acid Sequence

QVQLVESGGGVVQPGRSLRLSCAASGFTFTTYGMHWVRQAPGKGLEWVAV
IWYDGSNKYYGDSVKGRFTISRDNSKNTLYLQMNSLRVDDTAVYYCTRTH
GYTRSSDGFDYWGQGTLVTVSS (SEQ ID NO: 237)

HCDR1:GFTFTTYG (SEQ ID NO: 238)

HCDR2:IWYDGSNK (SEQ ID NO: 239)

HCDR3:TRTHGYTRSSDGFDY (SEQ ID NO: 240)

LCVR (V_{L}) Nucleotide Sequence

GACATCCAGATGACCCAGTCTCCATCCTCCCTGTCTGCATCTGTAGGAGA
CAGAGTCACCATCACTTGCCGGGCAAGTCAGAGCATTAGAAATGTTTTAG
GCTGGTTTCAGCAGAAACCAGGGAAAGCCCCTCAGCGCCTGATCTATGCT
GCATCCAGTTTGCAAAGTGGGGTCCCATCAAGGTTCAGCGGCAGTGGATC
TGGGACAGAATTCACTCTCACAATCAGCAGCCTACAGCCTGAAGATTTTG
CAACTTATTACTGTCTACAGCATAATTTTTACCCGCTCACTTTCGGCGGA
GGGACCAAGGTGGAGATCAAA (SEQ ID NO: 241)

LCVR (V_{L}) Amino Acid Sequence

DIQMTQSPSSLSASVGDRVTITCRASQSIRNVLGWFQQKPGKAPQRLIYA
ASSLQSGVPSRFSGSGSGTEFTLTISSLQPEDFATYYCLQHNFYPLTFGG
GTKVEIK (SEQ ID NO: 242)

LCDR1: QSIRNV (SEQ ID NO: 243)

LCDR2: AAS (SEQ ID NO: 244)

LCDR3: LQHNFYPLT (SEQ ID NO: 245)

### 69340

HCVR (V_{H}) Nucleotide Sequence

GAAGTGCAGCTGGTGGAGTCTGGGGGAGGCTTGGTACAGCCTGGCAGGTC
CCTGAGACTCTCCTGTGCAGCCTCTGGATTCACCTTTGATGATAAAGCCA
TGCACTGGGTCCGGCAAGTTCCAGGGAAGGGCCTGGAATGGATCTCAGGT
ATTAGTTGGAATAGTGGTACTATAGGCTATGCGGACTCTGTGAAGGGCCG
ATTCATCATCTCCAGAGACAACGCCAAGAACTCCCTGTATCTACAAATGA
ACAGTCTGAGAGCTGAGGACACGGCCTTGTATTACTGCGCAAAAGATGGA
GATACCAGTGGCTGGTACTGGTACGGTTTGGACGTCTGGGGCCAAGGGAC
CACGGTCACCGTCTCCTCA (SEQ ID NO: 246)

HCVR (V_{H}) Amino Acid Sequence

EVQLVESGGGLVQPGRSLRLSCAASGFTFDDKAMHWVRQVPGKGLEWISG
ISWNSGTIGYADSVKGRFIISRDNAKNSLYLQMNSLRAEDTALYYCAKDG
DTSGWYWYGLDVWGQGTTVTVSS (SEQ ID NO: 247)

HCDR1:GFTFDDKA (SEQ ID NO: 248)

HCDR2:ISWNSGTI (SEQ ID NO: 249)

HCDR3:AKDGDTSGWYWYGLDV (SEQ ID NO: 250)

LCVR (V_{L}) Nucleotide Sequence

GAAATTGTGTTGACACAGTCTCCTGCCACCCTGTCTTTGTCTCCAGGGGA
AAGAGCCACCCTCTCCTGCAGGGCCAGTCAGAGTGTTAGCAGCTACTTAG
CCTGGTACCAACAGAAACCTGGCCAGGCTCCCAGGCTCCTCATCCATGAT
GTATCCAACAGGGCCACTGGCATCCCAGCCAGGTTCAGTGGCAGTGGGTC
TGGGACAGACTTCACTCTCACCATCAGCAGTCTAGAGCCTGAAGATTTTG
TAGTTTATTACTGTCAGCAGCGTAGCGACTGGCCCATCACCTTCGGCCAA
GGGACACGACTGGAGATTAAA (SEQ ID NO: 251)

LCVR (V_{L}) Amino Acid Sequence

EIVLTQSPATLSLSPGERATLSCRASQSVSSYLAWYQQKPGQAPRLLIHD
VSNRATGIPARFSGSGSGTDFTLTISSLEPEDFVVYYCQQRSDWPITFGQ
GTRLEIK (SEQ ID NO: 252)

LCDR1: QSVSSY (SEQ ID NO: 253)

LCDR2: DVS (SEQ ID NO: 254)

LCDR3: QQRSDWPIT (SEQ ID NO: 255)

### 69331

HCVR (V_{H}) Nucleotide Sequence

CAGGTGCAGCTGGTGGAGTCTGGGGGAGGCGTGGTCCAGCCTGGGAGGTC
CCTGAGACTCTCCTGTATAGCCTCTGGATTCACCTTCAGTGTCTATGGCA
TTCACTGGGTCCGCCAGGCTCCAGGCAAGGGGCTGGAGTGGATGGCAGTA
ATATCACATGATGGAAATATTAAACACTATGCAGACTCCGTGAAGGGCCG
ATTCACCATCTCCAGAGACAATTCCAAGAACACGCTGTATCTTCAAATTA
ACAGCCTGAGAACTGAGGACACGGCTGTGTATTACTGTGCGAAAGATACC
TGGAACTCCCTTGATACTTTTGATATCTGGGGCCAAGGGACAATGGTCAC
CGTCTCTTCA (SEQ ID NO: 256)

HCVR (V_{H}) Amino Acid Sequence

QVQLVESGGGVVQPGRSLRLSCIASGFTFSVYGIHWVRQAPGKGLEWMAV
ISHDGNIKHYADSVKGRFTISRDNSKNTLYLQINSLRTEDTAVYYCAKDT
WNSLDTFDIWGQGTMVTVSS (SEQ ID NO: 257)

HCDR1:GFTFSVYG (SEQ ID NO: 258)

HCDR2:ISHDGNIK (SEQ ID NO: 259)

HCDR3:AKDTWNSLDTFDI (SEQ ID NO: 260)

LCVR (V_{L}) Nucleotide Sequence

GACATCCAGTTGACCCAGTCTCCATCCTCCCTGTCTGCATCTGTAGGAGA
CAGAGTCACCATCACTTGCTGGGCCAGTCAGGGCATTAGCAGTTATTTAG
CCTGGTATCAGCAAAAACCAGGGAAAGCCCCTAAGCTCCTGATCTATGCT
GCATCCACTTTGCAAAGTGGGGTCCCATCAAGGTTCAGCGGCAGTGGATC
TGGGACAGAATTCACTCTCACAATCAGCAGCCTGCAGCCTGAAGATTTTG
CAACTTATTACTGTCAACAGCTTAATAGTTACCCTCTCACTTTCGGCGGA
GGGACCAAGGTGGAGATCAAA (SEQ ID NO: 261)

LCVR (V_{L}) Amino Acid Sequence

DIQLTQSPSSLSASVGDRVTITCWASQGISSYLAWYQQKPGKAPKLLIYA
ASTLQSGVPSRFSGSGSGTEFTLTISSLQPEDFATYYCQQLNSYPLTFGG
GTKVEIK (SEQ ID NO: 262)

LCDR1: QGISSY (SEQ ID NO: 263)

LCDR2: AAS (SEQ ID NO: 264)

LCDR3: QQLNSYPLT (SEQ ID NO: 265)

### 69332

HCVR (V_{H}) Nucleotide Sequence

CAGGTCACCTTGAGGGAGTCTGGTCCCGCGCTGGTGAAACCCTCACAGAC
CCTCACACTGACCTGCACCTTCTCTGGATTCTCACTCAACACTTATGGGA
TGTTTGTGAGCTGGATCCGTCAGCCTCCAGGGAAGGCCCTAGAGTGGCTT
GCACACATTCATTGGGATGATGATAAATACTACAGCACATCTCTGAAGAC
CAGGCTCACCATCTCCAAGGACACCTCCAAAAACCAGGTGGTCCTTACAA
TGACCAACATGGACCCTGTGGACACAGCCACGTATTATTGTGCACGGGGG
CACAATAATTTGAACTACATCATCCACTGGGGCCAGGGAACCCTGGTCAC
CGTCTCCTCA (SEQ ID NO: 266)

HCVR (V_{H}) Amino Acid Sequence

QVTLRESGPALVKPSQTLTLTCTFSGFSLNTYGMFVSWIRQPPGKALEWL
AHIHWDDDKYYSTSLKTRLTISKDTSKNQVVLTMTNMDPVDTATYYCARG
HNNLNYIIHWGQGTLVTVSS (SEQ ID NO: 267)

HCDR1:GFSLNTYGMF (SEQ ID NO: 268)

HCDR2:IHWDDDK (SEQ ID NO: 269)

HCDR3:ARGHNNLNYIIH (SEQ ID NO: 270)

LCVR (V_{L}) Nucleotide Sequence

GCCATCCAGATGACCCAGTCTCCATCCTCCCTGTCTGCATCTGTAGGAGA
CAGAGTCACCATCACTTGCCGGGCAAGTCAGGGCATTAGAAATGATTTAG
GCTGGTATCAGCAGAAACCAGGGAAAGCCCCTAAGCTCCTGATCTATGCT
GCATCCACTTTACAAAGTGGGGTCCCATCAAGGTTCAGCGGCAGTGGATC
TGGCACAGATTTCACTCTCACCATCAGCAGCCTGCAGCCTGAAGATTTTG
CAACTTATTACTGTCTACAAGATTACAATTACCCATTCACTTTCGGCCCT
GGGACCAAAGTGGATATCAAA (SEQ ID NO: 271)

LCVR (V_{L}) Amino Acid Sequence

AIQMTQSPSSLSASVGDRVTITCRASQGIRNDLGWYQQKPGKAPKLLIYA
ASTLQSGVPSRFSGSGSGTDFTLTISSLQPEDFATYYCLQDYNYPFTFGP
GTKVDIK (SEQ ID NO: 272)

LCDR1: QGIRND (SEQ ID NO: 273)

LCDR2: AAS (SEQ ID NO: 274)

LCDR3: LQDYNYPFT (SEQ ID NO: 275)

### 69326

HCVR (V_{H}) Nucleotide Sequence

GAGGTGCAGCTGGTGGAGTCTGGGGGAGGCTTGGTACAGCCTGGAGGGTC
CCTGAGACTCTCCTGTGCAGTCTCTGGATTCATCTTCAGTAGTTATGAAA
TGAACTGGGTCCGCCAGGCTCCAGGGAAGGGGCTGGAGTGGGTTTCATAC
ATTAGTAGTAGTGGTAGTACCATATTCTACGCAGACTCTGTGAAGGGCCG
ATTCACCATCTCCAGAGACAACGCCAAGAACTCACTGTATCTGCAAATGA
ACAGCCTGAGAGCCGAGGACACGGCTGTTTATTACTGTGTGTCTGGAGTG
GTCCTTTTTGATGTCTGGGGCCAAGGGACAATGGTCACCGTCTCTTCA (
SEQ ID NO: 276)

HCVR (V_{H}) Amino Acid Sequence

EVQLVESGGGLVQPGGSLRLSCAVSGFIFSSYEMNWVRQAPGKGLEWVSY
ISSSGSTIFYADSVKGRFTISRDNAKNSLYLQMNSLRAEDTAVYYCVSGV
VLFDVWGQGTMVTVSS (SEQ ID NO: 277)

HCDR1:GFIFSSYE (SEQ ID NO: 278)

HCDR2:ISSSGSTI (SEQ ID NO: 279)

HCDR3:VSGVVLFDV (SEQ ID NO: 280)

LCVR (V_{L}) Nucleotide Sequence

GAAATAGTGATGACGCAGTCTCCAGCCACCCTGTCTGTGTCTCCGGGGGA
AAGAGCCACCCTCTCCTGCAGGGCCAGTCAGAGTGTTAGCAGCAACTTTG
CCTGGTACCAACAGAAACCTGGCCAGGCTCCCAGGCTCCTCATCTATAGT
GCATCCTCCAGGGCCACTGGTATCCCAGTCAGGTTCAGTGGCAGTGGGTC
TGGGACAGAGTTCACTCTCACCATCAGCAGCCTGCAGTCTGAAGATTTTG
CAGTTTATTACTGTCAGCAGTATAATATCTGGCCTCGGACGTTCGGCCAA
GGGACCAAGGTGGAAATCAAA (SEQ ID NO: 281)

LCVR (V_{L}) Amino Acid Sequence

EIVMTQSPATLSVSPGERATLSCRASQSVSSNFAWYQQKPGQAPRLLIYS
ASSRATGIPVRFSGSGSGTEFTLTISSLQSEDFAVYYCQQYNIWPRTFGQ
GTKVEIK (SEQ ID NO: 282)

LCDR1: QSVSSN (SEQ ID NO: 283)

LCDR2: SAS (SEQ ID NO: 284)

LCDR3: QQYNIWPRT (SEQ ID NO: 285)

### 69329

HCVR (V_{H}) Nucleotide Sequence

GAGGTGCAGCTGGTGGAGTCTGGGGGAGGCTTGGTCCAGCCTGGGGGGTC
CCTGAGACTCTCCTGTGCAGCCTCTGGATTCACCTTTAGTAACTATTGGA
TGACCTGGGTCCGCCAGGCTCCAGGGAAGGGGCTGGAGTGGGTGGCCAAC
ATAAAGGAAGATGGAAGTGAGAAAGACTATGTGGACTCTGTGAAGGGCCG
ATTCACCATCTCCAGAGACAACGCCAAGAACTCACTGTATCTGCAAATGA
ACAGCCTGAGAGGCGAGGACACGGCTGTGTATTACTGTGCGAGAGATGGG
GAGCAGCTCGTCGATTACTACTACTACTACGTTATGGACGTCTGGGGCCA
AGGGACCACGGTCACCGTCTCCTCA (SEQ ID NO: 286)

HCVR (V_{H}) Amino Acid Sequence

EVQLVESGGGLVQPGGSLRLSCAASGFTFSNYWMTWVRQAPGKGLEWVAN
IKEDGSEKDYVDSVKGRFTISRDNAKNSLYLQMNSLRGEDTAVYYCARDG
EQLVDYYYYYVMDVWGQGTTVTVSS (SEQ ID NO: 287)

HCDR1:GFTFSNYW (SEQ ID NO: 288)

HCDR2:IKEDGSEK (SEQ ID NO: 289)

HCDR3:ARDGEQLVDYYYYYVMDV (SEQ ID NO: 290)

LCVR (V_{L}) Nucleotide Sequence

GACATCCAGATGACCCAGTCTCCATCTTCCGTGTCTGCATCTGTAGGAGA
CAGAGTCACCATCACTTGTCGGGCGAGTCAGGGTATTAGCAGCTGGTTAG
CCTGGTATCAGCAGAAACCAGGGAAAGCCCCTAAGCTCCTGATCTATGCT
GCATCCAGTTTGCAAAGTGGGGTCCCATCAAGGTTCAGCGGCAGTGGATC
TGGGACAGATTTCACTCTCACCATCAGCAGCCTGCAGCCTGAAGATTTTG
CAACTTACTATTGTCAAAAGGCTAACAGTTTCCCGTACACTTTTGGCCAG
GGGACCAAGCTGGAGATCAAA (SEQ ID NO: 291)

LCVR (V_{L}) Amino Acid Sequence

DIQMTQSPSSVSASVGDRVTITCRASQGISSWLAWYQQKPGKAPKLLIYA
ASSLQSGVPSRFSGSGSGTDFTLTISSLQPEDFATYYCQKANSFPYTFGQ
GTKLEIK (SEQ ID NO: 292)

LCDR1: QGISSW (SEQ ID NO: 293)

LCDR2: AAS (SEQ ID NO: 294)

LCDR3: QKANSFPYT (SEQ ID NO: 295)

### 69323 (REGN16816 anti-hTfR scFv:hGAA)

HCVR (V_{H}) Nucleotide Sequence

GAAGTGCAGCTGGTGGAGTCTGGGGGAGGCTTGGTACAGCCTGGCAGGTC
CCTGAGACTCTCCTGTGCAGCCTCTGGATTCACCTTTGATGACTATGCCA
TGCACTGGGTCCGGCAAGCTCCAGGGAAGGGCCTGGAGTGGGTCTCAGGT
ATTAGTTGGAATAGTGGTTACATAGGCTATGCGGACTCTGTGAAGGGCCG
ATTCACCATCTCCAGAGACAACGCCGAGAACTCCCTACATCTGCAAATGA
ACAGTCTGAGAGCTGAGGACACGGCCTTGTATTACTGTGCAAGAGGGGGA
TCTACTCTGGTTCGGGGAGTTAAGGGAGGCTACTACGGTATGGACGTCTG
GGGCCAAGGGACCACGGTCACCGTCTCCTCA (SEQ ID NO: 296)

HCVR (V_{H}) Amino Acid Sequence

EVQLVESGGGLVQPGRSLRLSCAASGFTFDDYAMHWVRQAPGKGLEWVSG
ISWNSGYIGYADSVKGRFTISRDNAENSLHLQMNSLRAEDTALYYCARGG
STLVRGVKGGYYGMDVWGQGTTVTVSS (SEQ ID NO:297)

HCDR1:GFTFDDYA (SEQ ID NO: 298)

HCDR2:ISWNSGYI (SEQ ID NO: 299)

HCDR3:ARGGSTLVRGVKGGYYGMDV (SEQ ID NO: 300)

LCVR (V_{L}) Nucleotide Sequence

GACATCCAGATGACCCAGTCTCCATCCTCCCTGTCTGCATCTGTAGGAGA
CAGAGTCACCATCACTTGCCGGGCAAGTCAGAGCATAAGTAGCTATTTAA
ATTGGTATCAGCAGAAACCAGGTAAAGCCCCTAAGGTCCTGATCTATGCT
GCATCCAGTTTGCAAAGTGGGGTCCCATCAAGGTTCAGTGGCAGTGGATC
TGGGACAGATTTCACTCTCACCATCAGCAGTCTGCAACCTGAAGATTTTG
CAACTTACTACTGTCAACAGAGTTACAGTATTCCGCTCACTTTCGGCGGA
GGGACCAAGGTGGAGATCAAA (SEQ ID NO: 301)

LCVR (V_{L}) Amino Acid Sequence

DIQMTQSPSSLSASVGDRVTITCRASQSISSYLNWYQQKPGKAPKVLIYA
ASSLQSGVPSRFSGSGSGTDFTLTISSLQPEDFATYYCQQSYSIPLTFGG
GTKVEIK (SEQ ID NO: 302)

LCDR1: QSISSY (SEQ ID NO: 303)

LCDR2: AAS (SEQ ID NO: 304)

LCDR3: QQSYSIPLT (SEQ ID NO: 305)

### 69305

HCVR (V_{H}) Nucleotide Sequence

CAGGTGCAGCTGGTGGAGTCTGGGGGAGGCGTGGTCCAGCCTGGGAGGTC
CCTGAGACTCTCCTGTGCAGCGTCTGGATTCACCTTCAGTAGCTATGGCA
TGCACTGGGTCCGCCAGGCTCCAGGCAAGGGGCTGGAGTGGGTGGCAGTT
ATATGGTATGATGGAAGTAATAAATACTATGCAGACTCCGTGAAGGGCCG
ATTCACCATCTCCAGAGACATTTCCAAGAACACGCTGTATCTGCAAATGA
ACAGCCTGAGAGCCGAGGACACGGCCGTATATTACTGTGCGGGTCAACTG
GATCTCTTCTTTGACTACTGGGGCCAGGGAACCCTGGTCACCGTCTCCTC
A (SEQ ID NO: 306)

HCVR (V_{H}) Amino Acid Sequence

QVQLVESGGGVVQPGRSLRLSCAASGFTFSSYGMHWVRQAPGKGLEWVAV
IWYDGSNKYYADSVKGRFTISRDISKNTLYLQMNSLRAEDTAVYYCAGQL
DLFFDYWGQGTLVTVSS (SEQ ID NO: 307)

HCDR1:GFTFSSYG (SEQ ID NO: 308)

HCDR2:IWYDGSNK (SEQ ID NO: 309)

HCDR3: AGQLDLFFDY (SEQ ID NO: 310)

LCVR (V_{L}) Nucleotide Sequence

GACATCCAGATGACCCAGTCTCCATCCTCCCTGTCTGCATCTGTAGGAGA
CAGAGTCACCATCACTTGCCGGGCAAGTCAGAGCATTGACAGGTATTTAA
ATTGGTATCGGCAGAAACCAGGGAAAGCCCCTAAGCTCCTGATCTATACT
ACATCCAGTTTGCAAAGTGGGGTCCCATCAAGGTTCAGTGGCAGTGGATC
TGGGACAGATTTCACTCTCACCCTCAGCAGTCTGCAGCCTGAAGATTTTG
CAACTTACTACTGTCAGCAGAGTTACAGTCCCCCGCTCACTTTCGGCGGA
GGGACCAAGGTGGAGATCAAA (SEQ ID NO: 311)

LCVR (V_{L}) Amino Acid Sequence

DIQMTQSPSSLSASVGDRVTITCRASQSIDRYLNWYRQKPGKAPKLLIYT
TSSLQSGVPSRFSGSGSGTDFTLTLSSLQPEDFATYYCQQSYSPPLTFGG
GTKVEIK (SEQ ID NO: 312)

LCDR1: QSIDRY (SEQ ID NO: 313)

LCDR2: TTS (SEQ ID NO: 314)

LCDR3: QQSYSPPLT (SEQ ID NO: 315)

### 69307 (REGN16817 Anti-hTfR scFv:hGAA)

HCVR (V_{H}) Nucleotide Sequence

GAGGTGCAGCTGGTGGAGTCTGGGGGAGGCTTGGTCCAGCCTGGGGGGTC
CCTGAGACTCTCCTGTACAGCCTCTGGATTCACCTTTAGTAACTATTGGA
TGACCTGGGTCCGCCAGGCTCCAGGGAAGGGGCTGGAGTGGGTGGCCAAC
ATAAAGGAAGATGGAAGTGAGAAAGAGTATGTGGACTCTGTGAAGGGCCG
GTTCACCATCTCCAGAGACAACGCCAAGAATTCACTGTATCTGCAAATGA
ACAGCCTGAGAGGCGAGGACACGGCTGTATATTACTGTGCGAGAGATGGG
GAGCAGCTCGTCGATTACTATTACTACTACGTTATGGACGTCTGGGGCCA
AGGGACCACGGTCACCGTCTCCTCA (SEQ ID NO: 316)

HCVR (V_{H}) Amino Acid Sequence

EVQLVESGGGLVQPGGSLRLSCTASGFTFSNYWMTWVRQAPGKGLEWVAN
IKEDGSEKEYVDSVKGRFTISRDNAKNSLYLQMNSLRGEDTAVYYCARDG
EQLVDYYYYYVMDVWGQGTTVTVSS (SEQ ID NO: 317)

HCDR1:GFTFSNYW (SEQ ID NO: 318)

HCDR2:IKEDGSEK (SEQ ID NO: 319)

HCDR3:ARDGEQLVDYYYYYVMDV (SEQ ID NO: 320)

LCVR (V_{L}) Nucleotide Sequence

GACATCCAGATGACCCAGTCTCCATCTTCCGTGTCTGCATCTGTTGGAGA
CAGAGTCACCATCACTTGTCGGGCGAGTCAGGGTATTAGCAGCTGGTTAG
CCTGGTATCAGCAGAAACCAGGGAAAGCCCCTAAGCTCCTGATCTATGCT
GCATCCAGTTTGCAAAGTGGGGTCCCATCAAGGTTCAGCGGCAGTGGATC
TGGGACAGATTTCACTCTCACCATCAGCAGCCTGCAGCCTGAAGATTTTG
CAACTTACTATTGTCAAAAGGCTGACAGTCTCCCGTACGCTTTTGGCCAG
GGGACCAAGCTGGAGATCAAA (SEQ ID NO: 321)

LCVR (V_{L}) Amino Acid Sequence

DIQMTQSPSSVSASVGDRVTITCRASQGISSWLAWYQQKPGKAPKLLIYA
ASSLQSGVPSRFSGSGSGTDFTLTISSLQPEDFATYYCQKADSLPYAFGQ
GTKLEIK (SEQ ID NO: 322)

LCDR1: QGISSW (SEQ ID NO: 323)

LCDR2: AAS (SEQ ID NO: 324)

LCDR3: QKADSLPYA (SEQ ID NO: 325)

### 12795B

HCVR (V_{H}) Nucleotide Sequence

GAGGTGCAGCTGGTGGAGTCTGGGGGAGGCTTGGTTCAGCCTGGGGGGTC
CCTGAGACTCTCCTGTGCAACCTCTGGATTCACCTTTACCAGCTATGACA
TGAAGTGGGTCCGCCAGGCTCCAGGGCTGGGCCTGGAGTGGGTCTCAGCT
ATTAGTGGTAGTGGTGGTAACACATACTACGCAGACTCCGTGAAGGGCCG
GTTCACCATCTCCAGAGACAATTCCAGGAACACGCTGTATCTGCAAATGA
ACAGCCTGAGAGCCGAGGACACGGCCGTATATTACTGTACGAGGTCCCAT
GACTTCGGTGCCTTCGACTACTTTGACTACTGGGGCCAGGGAACCCTGGT
CACCGTCTCCTCA (SEQ ID NO: 326)

HCVR (V_{H}) Amino Acid Sequence

EVQLVESGGGLVQPGGSLRLSCATSGFTFTSYDMKWVRQAPGLGLEWVSA
ISGSGGNTYYADSVKGRFTISRDNSRNTLYLQMNSLRAEDTAVYYCTRSH
DFGAFDYFDYWGQGTLVTVSS (SEQ ID NO: 327)

HCDR1:GFTFTSYD (SEQ ID NO: 328)

HCDR2:ISGSGGNT (SEQ ID NO: 329)

HCDR3:TRSHDFGAFDYFDY (SEQ ID NO: 330)

LCVR (V_{L}) Nucleotide Sequence

GACATCCAGATGACCCAGTCTCCATCCTCCCTGTCTGCATCTGTGGGAGA
CAGAGTCACCATCACTTGCCGGGCAAGTCAGGGCATTAGAGATCATTTTG
GCTGGTATCAGCAGAAACCAGGGAAAGCCCCTAAGCGCCTGATCTATGCT
GCATCCAGTTTGCACAGTGGGGTCCCATCAAGGTTCAGCGGCAGTGGATC
TGGGACAGAATTCACTCTCACAATCAGCAGCTTGCAGCCTGAAGATTTTG
CAACCTATTACTGTCTACAGTATGATACTTACCCGCTCACTTTCGGCGGA
GGGACCAAGGTGGAGATCAAA (SEQ ID NO: 331)

LCVR (V_{L}) Amino Acid Sequence

DIQMTQSPSSLSASVGDRVTITCRASQGIRDHFGWYQQKPGKAPKRLIYA
ASSLHSGVPSRFSGSGSGTEFTLTISSLQPEDFATYYCLQYDTYPLTFGG
GTKVEIK (SEQ ID NO: 332)

LCDR1: QGIRDH (SEQ ID NO: 333)

LCDR2: AAS (SEQ ID NO: 334)

LCDR3: LQYDTYPLT (SEQ ID NO: 335)

### 12798B (REGN17078 Fab; REGN17072 scFv; REGN16818 Anti-hTfR scFv:hGAA)

HCVR (V_{H}) Nucleotide Sequence

GAAGTGCAGCTGGTGGAGTCTGGGGGAGACTTGGTACAGCCTGGCAGGTC
CCTGAGACTCTCCTGTGCAGCCTCTGGATTCACCTTTGATGATTATGCCA
TGCACTGGGTCCGGCAAGCTCCAGGGAAGGGCCTGGAGTGGGTCTCAGGT
ATTAGTTGGAATAGTGCTACCAGAGTCTATGCGGACTCTGTGAAGGGCCG
ATTCACCATCTCCAGAGACAACGCCAAGAATTTCCTGTATCTGCAAATGA
ACAGTCTGAGATCTGAGGACACGGCCTTGTATCACTGTGCAAAAGATATG
GATATCTCGCTAGGGTACTACGGTTTGGACGTCTGGGGCCAAGGGACCAC
GGTCACCGTCTCCTCA (SEQ ID NO: 336)

HCVR (V_{H}) Amino Acid Sequence

EVQLVESGGDLVQPGRSLRLSCAASGFTFDDYAMHWVRQAPGKGLEWVSG
ISWNSATRVYADSVKGRFTISRDNAKNFLYLQMNSLRSEDTALYHCAKDM
DISLGYYGLDVWGQGTTVTVSS (SEQ ID NO: 337)

HCDR1:GFTFDDYA (SEQ ID NO: 338)

HCDR2:ISWNSATR (SEQ ID NO: 339)

HCDR3:AKDMDISLGYYGLDV (SEQ ID NO: 340)

LCVR (V_{L}) Nucleotide Sequence

GAAATAGTGATGACGCAGTCTCCAGCCACCCTGTCTGTGTCTCCAGGGGA
AAGAGCCACCCTCTCCTGCAGGGCCAGTCAGACTGTTAGCAGCAACTTAG
CCTGGTATCAGCAGAAACCTGGCCAGGCTCCCAGGCTCCTCATCTATGGT
TCATCCTCCAGGGCCACTGGTATCCCAGCCAGGTTCAGTGGCAGTGGGTC
TGGGACAGAGTTCACTCTCACCATCAGCAGCCTGCAGTCTGAAGATTTTG
CAGTTTATTACTGTCAGCAGTATAATAACTGGCCTCCCTACACTTTTGGC
CAGGGGACCAAGCTGGAGATCAAA (SEQ ID NO: 341)

LCVR (V_{L}) Amino Acid Sequence

EIVMTQSPATLSVSPGERATLSCRASQTVSSNLAWYQQKPGQAPRLLIYG
SSSRATGIPARFSGSGSGTEFTLTISSLQSEDFAVYYCQQYNNWPPYTFG
QGTKLEIK (SEQ ID NO: 342)

LCDR1: QTVSSN (SEQ ID NO: 343)

LCDR2: GSS (SEQ ID NO: 344)

LCDR3: QQYNNWPPYT (SEQ ID NO: 345)

### 12799B (REGN17079 Fab; REGN17073 scFv; REGN16819 Anti-hTfR scFv:hGAA)

HCVR (V_{H}) Nucleotide Sequence

CAGATCACCTTGAAGGAGTCTGGTCCTACGCTGGTGAAACCCACACAGAC
CCTCACGCTGACCTGCACCTTCTCTGGGTTCTCACTCAGCACTAGTGGAG
TGGGTGTGGTCTGGATCCGTCAGCCCCCCGGAAAGGCCCTGGAGTGGCTT
GCACTCATTTATTGGAATGATCATAAGCGGTACAGCCCATCTCTGGGGAG
CAGGCTCACCATCACCAAGGACACCTCCAAAAACCAGGTGGTCCTTACAA
TGACCAACATGGACCCTGTGGACACAGCCACATATTACTGTGCACACTAC
AGTGGGAGCTATTCCTACTACTACTATGGTTTGGACGTCTGGGGCCAAGG
GACCACGGTCACCGTCTCCTCA (SEQ ID NO: 346)

HCVR (V_{H}) Amino Acid Sequence

QITLKESGPTLVKPTQTLTLTCTFSGFSLSTSGVGVVWIRQPPGKALEWL
ALIYWNDHKRYSPSLGSRLTITKDTSKNQVVLTMTNMDPVDTATYYCAHY
SGSYSYYYYGLDVWGQGTTVTVSS (SEQ ID NO: 347)

HCDR1:GFSLSTSGVG (SEQ ID NO: 348)

HCDR2:IYWNDHK (SEQ ID NO: 349)

HCDR3:AHYSGSYSYYYYGLDV (SEQ ID NO: 350)

LCVR (V_{L}) Nucleotide Sequence

GACATCCAGATGACCCAGTCTCCATCTTCCGTGTCTGCATCTGTAGGAGA
CAGAGTCACCATCACTTGTCGGGCGAGTCAGGGTATTGCCAGCTGGTTAG
CCTGGTATCAGCAGAAACCAGGGAAAGCCCCTGAGCTCCTGATCTATGCT
GCATCCAGTTTGCAAGGTGGGGTCCCATCAAGGTTCAGCGGCAGTGGATC
TGGGACAGATTTCACTCTCACCATCAGCAGCCTGCAGCCTGAAGATTTTG
CAATTTACTATTGTCAACAGGCTAACTATTTCCCGTGGACGTTCGGCCAA
GGGACCAAGGTGGAAATCAAA (SEQ ID NO: 351)

LCVR (V_{L}) Amino Acid Sequence

DIQMTQSPSSVSASVGDRVTITCRASQGIASWLAWYQQKPGKAPELLIYA
ASSLQGGVPSRFSGSGSGTDFTLTISSLQPEDFAIYYCQQANYFPWTFGQ
GTKVEIK (SEQ ID NO: 352)

LCDR1: QGIASW (SEQ ID NO: 353)

LCDR2: AAS (SEQ ID NO: 354)

LCDR3: QQANYFPWT (SEQ ID NO: 355)

### 12801B

HCVR (V_{H}) Nucleotide Sequence

GAGGTGCAGCTGTTGGAGTCTGGGGGAGCCTTGGTACAGCCTGGGGGGTC
CCTGAGACTCTCCTGTGCAGCCTCTGGATTCACCTTTACCTCCTATGCCA
TGCACTGGGTCCGCCAGGCTCCAGGGAAGGGTCTGGAGTGGGTCTCATCT
ATTAGAGGTAGTGGTGGTGGCACATACTCCGCAGACTCCGTGAAGGGCCG
GTTCACCATCTCCAGAGACAATTCCAGGGACACTCTATATCTGCAAATGA
ACAGTGTGAGAGCCGAGGACACGGCCGTTTATTACTGTGCGAGGTCCCAT
GACTACGGTGCCTTCGACTTCTTTGACTACTGGGGCCAGGGAACCCTGGT
CACCGTCTCCTCA (SEQ ID NO: 356)

HCVR (V_{H}) Amino Acid Sequence

EVQLLESGGALVQPGGSLRLSCAASGFTFTSYAMHWVRQAPGKGLEWVSS
IRGSGGGTYSADSVKGRFTISRDNSRDTLYLQMNSVRAEDTAVYYCARSH
DYGAFDFFDYWGQGTLVTVSS (SEQ ID NO: 357)

HCDR1:GFTFTSYA (SEQ ID NO: 358)

HCDR2:IRGSGGGT (SEQ ID NO: 359)

HCDR3:ARSHDYGAFDFFDY (SEQ ID NO: 360)

LCVR (V_{L}) Nucleotide Sequence

GACATCCAGATGACCCAGTCTCCATCCTCCCTGTCTGCATCTGTAGGAGA
CAGAGTCACCATCACTTGCCGGGCAAGTCAGGGCATTAGAACTGATTTAG
GCTGGTATCAGCAGAAACCAGGGAAAGCCCCTAAGCGCCTGATCTATGCT
GCATCCAGTTTGCAAAGTGGGGTCCCATCAAGGTTCAGCGGCAGTGGATC
TGGGACAGAATTCACTCTCACAATCAGCAGCCTGCGGCCTGAAGATTTTG
CAACTTTTTACTGTCTACAGTATAATAGTTACCCGCTCACTTTCGGCGGA
GGGACCAAGGTGGAGATCAAA (SEQ ID NO: 361)

LCVR (V_{L}) Amino Acid Sequence

DIQMTQSPSSLSASVGDRVTITCRASQGIRTDLGWYQQKPGKAPKRLIYA
ASSLQSGVPSRFSGSGSGTEFTLTISSLRPEDFATFYCLQYNSYPLTFGG
GTKVEIK (SEQ ID NO: 362)

LCDR1: QGIRTD (SEQ ID NO: 363)

LCDR2: AAS (SEQ ID NO: 364)

LCDR3: LQYNSYPLT (SEQ ID NO: 365)

### 12802B (REGN16820 Anti-hTfR scFv:hGAA)

HCVR (V_{H}) Nucleotide Sequence

CAGGTGCAGCTGGTGGAGTCTGGGGGAGGCTTGGTCAAGCCTGGAGGGTC
CCTGAGACTCTCCTGTGCAGCCTCTGGATTCACCTTCAGTGACTACTTCA
TGAGCTGGATCCGCCAGGCTCCAGGGAAGGGGCTGGAGTGGGTTTCATAC
ATTAGTAGTACTGGTAGTACCATAAATTATGCAGACTCTGTGAAGGGCCG
ATTCACCATCTCCAGGGACAATGTCAAGAATTCACTGTATCTGCAAATGA
CCAGCCTGAGAGTCGAGGACACGGCCGTGTATTACTGTACGAGAGATAAC
TGGAACTATGAATACTGGGGCCAGGGAACCCTGGTCACCGTCTCCTCA (
SEQ ID NO: 366)

HCVR (V_{H}) Amino Acid Sequence

QVQLVESGGGLVKPGGSLRLSCAASGFTFSDYFMSWIRQAPGKGLEWVSY
ISSTGSTINYADSVKGRFTISRDNVKNSLYLQMTSLRVEDTAVYYCTRDN
WNYEYWGQGTLVTVSS (SEQ ID NO: 367)

HCDR1:GFTFSDYF (SEQ ID NO: 368)

HCDR2:ISSTGSTI (SEQ ID NO: 369)

HCDR3:TRDNWNYEY (SEQ ID NO: 370)

LCVR (V_{L}) Nucleotide Sequence

GAAATAGTGATGACGCAGTCTCCAGCCACCCTGTCTGTGTCTCCAGGGGA
AAGAGCCACCCTCTCCTGCAGGGCCAGTCAGAGTGTTAGCATCAACTTAG
CCTGGTACCAGCAGAAACCTGGCCAGGCTCCCAGGCTCCTCATCTTTGTT
GCATCCACCAGGGCCACTGGTATCCCAGCCAGGTTCAGTGGCAGTGGGTC
TGGGACAGAGTTCACTCTCACCATCAGCAGCCTGCAGTCTGAAGATTTTG
CAACTTATTACTGTCAGCAGTATGATATCTGGCCGTACACTTTTGGCCAG
GGGACCAAGCTGGAGATCAAA (SEQ ID NO: 371)

LCVR (V_{L}) Amino Acid Sequence

EIVMTQSPATLSVSPGERATLSCRASQSVSINLAWYQQKPGQAPRLLIFV
ASTRATGIPARFSGSGSGTEFTLTISSLQSEDFATYYCQQYDIWPYTFGQ
GTKLEIK (SEQ ID NO: 372)

LCDR1: QSVSIN (SEQ ID NO: 373)

LCDR2: VAS (SEQ ID NO: 374)

LCDR3: QQYDIWPYT (SEQ ID NO: 375)

### 12808B

HCVR (V_{H}) Nucleotide Sequence

CAGCTGCAGCTGCAGGAGTCGGGCCCAGGACTGGTGAAGCCTTCGGAGAC
CCTGTCCCTCACCTGCACTGTGTCTGGTGAATCCATCAGCAGTAATACTT
ACTACTGGGGCTGGATCCGCCAGCCCCCAGGGAAGGGGCTGGAATGGATT
GGGAGTATCGATTATAGTGGGACCACCAATTATAACCCGTCCCTCAAGAG
TCGAGTCACCATATCCGTAGACACGTCCAGGAATCACTTCTCCCTGAGGC
TGAGGTCTGTGACCGCCGCAGACACGGCTGTGTATTACTGTGCGAGAGAG
TGGGGAAACTACGGCTACTATTACGGTATGGACGTTTGGGGCCAAGGGAC
CACGGTCACCGTCTCCTCA (SEQ ID NO: 376)

HCVR (V_{H}) Amino Acid Sequence

QLQLQESGPGLVKPSETLSLTCTVSGESISSNTYYWGWIRQPPGKGLEWI
GSIDYSGTTNYNPSLKSRVTISVDTSRNHFSLRLRSVTAADTAVYYCARE
WGNYGYYYGMDVWGQGTTVTVSS (SEQ ID NO: 377)

HCDR1:GESISSNTYY (SEQ ID NO: 378)

HCDR2:IDYSGTT (SEQ ID NO: 379)

HCDR3:AREWGNYGYYYGMDV (SEQ ID NO: 380)

LCVR (V_{L}) Nucleotide Sequence

GACATCCAGATGACCCAGTCTCCATCCTCCCTGTCTGCATCTGTAGGAGA
CAGAGTCACCATCAATTGCCGGGCAAGTCAGGGCATTAGAAATGATTTAG
GCTGGTATCAGCAGAAACCAGGGAAAGCCCCTAAGCGCCTGATCTATGCT
GCATCCAGTTTGCAAAGTGGGGTCCCATTAAGGTTCAGTGGCAGTGGATC
TGGGACAGAATTCACTCTCACAATCAACAACCTGCAGCCTGAAGATTTTG
CAACTTATTACTGTCTATCGCATAATAGTTACCCGTGGACGTTCGGCCAA
GGGACCAAGGTGGAAATCAAA (SEQ ID NO: 381)

LCVR (V_{L}) Amino Acid Sequence

DIQMTQSPSSLSASVGDRVTINCRASQGIRNDLGWYQQKPGKAPKRLIYA
ASSLQSGVPLRFSGSGSGTEFTLTINNLQPEDFATYYCLSHNSYPWTFGQ
GTKVEIK (SEQ ID NO: 382)

LCDR1: QGIRND (SEQ ID NO: 383)

LCDR2: AAS (SEQ ID NO: 384)

LCDR3: LSHNSYPWT (SEQ ID NO: 385)

### 12812B (REGN16821 Anti-hTfR scFv:hGAA)

HCVR (V_{H}) Nucleotide Sequence

CAGGTGCAGCTGGTGCAGTCTGGGGCTGAGGTGAAGAAGCCTGGGTCCTC
GGTGAGGGTCTCCTGCAAGGCTTCTAGAGGCACCTTCAGCAGCTATGCTA
TCAGCTGGGTGCGACAGGCCCCTGGACAAGGCCTTGAGTGGATGGGAGGG
ATCATCCCCATCTTTGGTACAGCAAACTACGCACAGAAGTTCCTGGCCAG
AGTCACGATTACCGCGGACGAATCCACGAGCACAGCCTACATGGAGCTGA
GCAGCCTGAGATCTGAGGACACGGCCGTGTATTACTGTGCGAGAGAGAAG
GGGTGGAACTACTTTGACTACTGGGGCCAGGGAACCCTGGTCACCGTCTC
CTCA (SEQ ID NO: 386)

HCVR (V_{H}) Amino Acid Sequence

QVQLVQSGAEVKKPGSSVRVSCKASRGTFSSYAISWVRQAPGQGLEWMGG
IIPIFGTANYAQKFLARVTITADESTSTAYMELSSLRSEDTAVYYCAREK
GWNYFDYWGQGTLVTVSS (SEQ ID NO: 387)

HCDR1:RGTFSSYA (SEQ ID NO: 388)

HCDR2:IIPIFGTA (SEQ ID NO: 389)

HCDR3:AREKGWNYFDY (SEQ ID NO: 390)

LCVR (V_{L}) Nucleotide Sequence

GACATCCAGATGACCCAGTCTCCACCTTCCGTGTCTGCATCTGTAGGAGA
CAGAGTCACCATCACTTGTCGGGCGAGTCAGGGTATTAGCAGCTGGTTAG
CCTGGTATCAGCAGAAACCAGGGAAAGCCCCTAAACTCCTGATCTATGCT
GCATCCAGTTTGCAAAGTGGGGTCCCATCAAGGTTCAGCGGCAGTGGATC
TGGGACAGATTTCACTCTCACCATCAGCAGCCTGCAGCCTGAAGATTTTG
CAACTTACTATTGTCAACAGGCTAACAGTTTCCCTCGGACGTTCGGCCAA
GGGACCAAGGTGGAAATCAAA (SEQ ID NO: 391)

LCVR (V_{L}) Amino Acid Sequence

DIQMTQSPPSVSASVGDRVTITCRASQGISSWLAWYQQKPGKAPKLLIYA
ASSLQSGVPSRFSGSGSGTDFTLTISSLQPEDFATYYCQQANSFPRTFGQ
GTKVEIK (SEQ ID NO: 392)

LCDR1: QGISSW (SEQ ID NO: 393)

LCDR2: AAS (SEQ ID NO: 394)

LCDR3: QQANSFPRT (SEQ ID NO: 395)

### 12816B

HCVR (V_{H}) Nucleotide Sequence

CAGGTGCAGCTGGTGGAGTCTGGGGGAGGCTTGGTCAAGCCTGGAGGGTC
CCTGAGACTCTCCTGTGCAGCCTCTGGATTCACCTTCAGTGACTACTACA
TGAACTGGATCCGCCAGGCTCCAGGGAAGGGGCTGGAGTGGGTTTCATAC
ATTAGTAGTAGTGGGACTACCATATACTACGCAGACTCTGTGAAGGGCCG
ATTCACCATCTCCAGGGACAACGCCAAGAAATCACTGTATCTGGAGATGA
ACAGCCTCAGAGCCGAGGACACGGCCGTGTACTACTGTGCGAGAGAGGGG
TACGGTAATGACTACTATTACTACGGTATAGACGTCTGGGGCCAAGGGAC
CACGGTCACCGTCTCCTCA (SEQ ID NO: 396)

HCVR (V_{H}) Amino Acid Sequence

QVQLVESGGGLVKPGGSLRLSCAASGFTFSDYYMNWIRQAPGKGLEWVSY
ISSSGTTIYYADSVKGRFTISRDNAKKSLYLEMNSLRAEDTAVYYCAREG
YGNDYYYYGIDVWGQGTTVTVSS (SEQ ID NO: 397)

HCDR1:GFTFSDYY (SEQ ID NO: 398)

HCDR2:ISSSGTTI (SEQ ID NO: 399)

HCDR3:AREGYGNDYYYYGIDV (SEQ ID NO: 400)

LCVR (V_{L}) Nucleotide Sequence

GATATTGTGATGACTCAGTCTCCACTCTCCCTGCCCGTCACCCCTGGAGA
GCCGGCCTCCATCTCCTGCAGGTCTAGTCAGAGCCTCCTGCATGGTAATG
GATACAACTATTTGACTTGGTACCTGCAGAAGCCAGGGCAGTCTCCACAG
CTCCTGATCTATTTGGGTTCTAATCGGGCCTCCGGGGTCCCTGACAGGTT
CAGTGGCAGTGGATCAGGCACAGATTTTACACTGAAAATAAGCAGAGTGG
AGGCTGAGGATGTTGGGGTTTATTACTGCATGCAAGCTCTACAAACTCCG
TACACTTTTGGCCAGGGGACCAAGCTGGAGATCAAA (SEQ IDNO: 40
1)

LCVR (V_{L}) Amino Acid Sequence

DIVMTQSPLSLPVTPGEPASISCRSSQSLLHGNGYNYLTWYLQKPGQSPQ
LLIYLGSNRASGVPDRFSGSGSGTDFTLKISRVEAEDVGVYYCMQALQTP
YTFGQGTKLEIK (SEQ ID NO: 402)

LCDR1: QSLLHGNGYNY (SEQ ID NO: 403)

LCDR2: LGS (SEQ ID NO: 404)

LCDR3: MQALQTPYT (SEQ ID NO: 405)

### 12833B

HCVR (V_{H}) Nucleotide Sequence

CAGGTGCAGCTGGTGGAGTCTGGGGGAGGCGTGGTCCAGCCTGGGAGGTC
CCTGAGACTCTCCTGTGCAGCCTCTGGATTCACCTTCAGTAGCTTTGGCA
TGCACTGGGTCCGCCAGGCTCCAGGCAAGGGGCTGGAGTGGGTGATATTT
ATATCATATGATGGAAGTGATAAATACTATGCAGACTCCGTGAAGGGCCG
ATTCGCCATCTCCAGAGACAGTTCCAAGAACACGCTATATCTGCAAATGA
ACAGCCTGAGAGCTGAGGACACGGCTGTGTATTACTGTGCGAAAGAAAAC
GGTATTTTGACTGATTCCTACGGTATGGACGTCTGGGGCCAAGGGACCAC
GGTCACCGTCTCCTCA (SEQ ID NO: 406)

HCVR (V_{H}) Amino Acid Sequence

QVQLVESGGGVVQPGRSLRLSCAASGFTFSSFGMHWVRQAPGKGLEWVIF
ISYDGSDKYYADSVKGRFAISRDSSKNTLYLQMNSLRAEDTAVYYCAKEN
GILTDSYGMDVWGQGTTVTVSS (SEQ ID NO: 407)

HCDR1:GFTFSSFG (SEQ ID NO: 408)

HCDR2:ISYDGSDK (SEQ ID NO: 409)

HCDR3:AKENGILTDSYGMDV (SEQ ID NO: 410)

LCVR (V_{L}) Nucleotide Sequence

GACATCCAGATGACCCAGTCTCCATCCTCCCTGTCTGCATCTGTAGGAGA
CAGAGTCACCATCACTTGCCGGGCAAGTCAGAGCATTAGCAGCTATTTAA
ATTGGTATCAGCAGAAACCAGGGAAAGCCCCTAAGCTCCTGATCTATGCT
GCATCCAGTTTGCAAAGTGGGGTCCCGTCAAGGTTCAGTGGCAGTGGATC
TGGGACAGATTTCACTCTCACCATCAGCAGTCTGCAACCTGAAGATTTTG
CAACTTACTACTGTCAACAGAGTTACAGTACCCCTCCGATCACCTTCGGC
CAAGGGACACGACTGGAGATTAAA (SEQ ID NO: 411)

LCVR (V_{L}) Amino Acid Sequence

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPITFG
QGTRLEIK (SEQ ID NO: 412)

LCDR1: QSISSY (SEQ ID NO: 413)

LCDR2: AAS (SEQ ID NO: 414)

LCDR3: QQSYSTPPIT (SEQ ID NO: 415)

### 12834B

HCVR (V_{H}) Nucleotide Sequence

CAGGTTCAGCTGGTGCAGTCTGGAGCTGAGGTGAAGAAGCCTGGGGCCTC
TGTGAAGGTCTCCTGCAAGGCTTCTGGTTACACCTTTACCAGCTATGGTA
TCAGCTGGGTGCGACAGGCCCCTGGACAAGGGCTTGAGTGGATGGGATGG
ATCAGTGTTTACCATGGTAACACAAACTATGCACAGAAGTTCCAGGGCAG
AGTCACCATGACCACAGACACATCCACGAGCACAGCCTACATGGAGCTGA
GGAGCCTGAGATCTGACGACACGGCCGTGTATTACTGTGCGAGAGAGGGG
TATTACGATTTTTGGAGTGGTTATTACCCTTTTGACTACTGGGGCCAGGG
AACCCTGGTCACCGTCTCCTCA (SEQ ID NO: 416)

HCVR (V_{H}) Amino Acid Sequence

QVQLVQSGAEVKKPGASVKVSCKASGYTFTSYGISWVRQAPGQGLEWMGW
ISVYHGNTNYAQKFQGRVTMTTDTSTSTAYMELRSLRSDDTAVYYCAREG
YYDFWSGYYPFDYWGQGTLVTVSS (SEQ ID NO: 417)

HCDR1:GYTFTSYG (SEQ ID NO: 418)

HCDR2:ISVYHGNT (SEQ ID NO: 419)

HCDR3:AREGYYDFWSGYYPFDY (SEQ ID NO: 420)

LCVR (V_{L}) Nucleotide Sequence

GACATCCAGATGACCCAGTCTCCATCCTCCCTGTCTGCATCTGTAGGAGA
CAGAGTCACCATCACTTGCCGGGCAAGTCAGAGCATTAGCAGCTATTTAA
ATTGGTATCAGCAGAAACCAGGGAAAGCCCCTAAGCTCCTGATCTATGCT
GCATCCAGTTTGCAAAGTGGGGTCCCGTCAAGGTTCAGTGGCAGTGGATC
TGGGACAGATTTCACTCTCACCATCAGCAGTCTGCAACCTGAAGATTTTG
CAACTTACTACTGTCAACAGAGTTACAGTACCCCTCCGATCACCTTCGGC
CAAGGGACACGACTGGAGATTAAA (SEQ ID NO: 421)

LCVR (V_{L}) Amino Acid Sequence

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPITFG
QGTRLEIK (SEQ ID NO: 422)

LCDR1: QSISSY (SEQ ID NO: 423)

LCDR2: AAS (SEQ ID NO: 424)

LCDR3: QQSYSTPPIT (SEQ ID NO: 425)

### 12835B

HCVR (V_{H}) Nucleotide Sequence

GAGGTGCAGCTGGTGGAGTCTGGGGGAGGCTTGATACAACCTGGAGGGTC
CCTGAGACTCTCCTGTGAAGCCTCTGGATTCACCTTCAGAAATTATGAAA
TGAATTGGGTCCGCCAGGCTCCAGGGAAGGGGCTGGAGTGGGTTTCATAT
ATTAGTAGTAGTGGTAATATGAAAGACTACGCAGAGTCTGTGAAGGGCCG
ATTCACCATCTCCAGAGACAATGTCAAGAATTCACTGCAGCTGCAAATGA
ACAGCCTGAGAGTCGAGGACACGGCTGTTTATTACTGTGCGAGAGACGAG
TTTCCTTACGGAATGGACGTCTGGGGCCAAGGGACCACGGTCACCGTCTC
CTCA (SEQ ID NO: 426)

HCVR (V_{H}) Amino Acid Sequence

EVQLVESGGGLIQPGGSLRLSCEASGFTFRNYEMNWVRQAPGKGLEWVSY
ISSSGNMKDYAESVKGRFTISRDNVKNSLQLQMNSLRVEDTAVYYCARDE
FPYGMDVWGQGTTVTVSS (SEQ ID NO: 427)

HCDR1:GFTFRNYE (SEQ ID NO: 428)

HCDR2:ISSSGNMK (SEQ ID NO: 429)

HCDR3:ARDEFPYGMDV (SEQ ID NO: 430)

LCVR (V_{L}) Nucleotide Sequence

GACATCCAGATGACCCAGTCTCCATCCTCCCTGTCTGCATCTGTAGGAGA
CAGAGTCACCATCACTTGCCGGGCAAGTCAGAGCATTAGCAGCTATTTAA
ATTGGTATCAGCAGAAACCAGGGAAAGCCCCTAAGCTCCTGATCTATGCT
GCATCCAGTTTGCAAAGTGGGGTCCCGTCAAGGTTCAGTGGCAGTGGATC
TGGGACAGATTTCACTCTCACCATCAGCAGTCTGCAACCTGAAGATTTTG
CAACTTACTACTGTCAACAGAGTTACAGTACCCCTCCGATCACCTTCGGC
CAAGGGACACGACTGGAGATTAAA (SEQ ID NO: 431)

LCVR (V_{L}) Amino Acid Sequence

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPITFG
QGTRLEIK (SEQ ID NO: 432)

LCDR1: QSISSY (SEQ ID NO: 433)

LCDR2: AAS (SEQ ID NO: 434)

LCDR3: QQSYSTPPIT (SEQ ID NO: 435)

### 12847B (REGN17083 Anti-hTfR Fab; REGN17077 Anti-hTfR scFv; REGN16826 Anti-hTfR scFv:hGAA)

HCVR (V_{H}) Nucleotide Sequence

GAAGTGCAGCTGGTGGAGTCTGGGGGAGGCTTGGTTCAGCCTGGCAGGTC
CCTGAGACTCTCCTGTGCAGCCTCTGGATTCACCTTTGATGATTATGCCA
TGAACTGGGTCCGGCAAGCTCCAGGGAAGGGCCTGGAGTGGGTCTCAGGT
ATTAGTTGGAGTAGTGGTAGCATGGACTATGCGGACTCTGTGAAGGGCCG
ATTCACCATCTCCAGAGACAACGCCAAAAACTCCCTGTATCTGCAAATGA
ACAGTCTGAGAACTGAGGACACGGCCTTATATTACTGTGCAAAAGCTAGG
GAAGTTGGAGACTACTACGGTATGGACGTCTGGGGCCAAGGGACCACGGT
CACCGTCTCCTCA (SEQ ID NO: 436)

HCVR (V_{H}) Amino Acid Sequence

EVQLVESGGGLVQPGRSLRLSCAASGFTFDDYAMNWVRQAPGKGLEWVSG
ISWSSGSMDYADSVKGRFTISRDNAKNSLYLQMNSLRTEDTALYYCAKAR
EVGDYYGMDVWGQGTTVTVSS (SEQ ID NO: 437)

HCDR1:GFTFDDYA (SEQ ID NO: 438)

HCDR2:ISWSSGSM (SEQ ID NO: 439)

HCDR3:AKAREVGDYYGMDV (SEQ ID NO: 440)

LCVR (V_{L}) Nucleotide Sequence

GACATCCAGATGACCCAGTCTCCATCCTCCCTGTCTGCATCTGTAGGAGA
CAGAGTCACCATCACTTGCCGGGCAAGTCAGAGCATTAGCAGCTATTTAA
ATTGGTATCAGCAGAAACCAGGGAAAGCCCCTAAGCTCCTGATCTATGCT
GCATCCAGTTTGCAAAGTGGGGTCCCGTCAAGGTTCAGTGGCAGTGGATC
TGGGACAGATTTCACTCTCACCATCAGCAGTCTGCAACCTGAAGATTTTG
CAACTTACTACTGTCAACAGAGTTACAGTACCCCTCCGATCACCTTCGGC
CAAGGGACACGACTGGAGATTAAA (SEQ ID NO: 441)

LCVR (V_{L}) Amino Acid Sequence

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPITFG
QGTRLEIK (SEQ ID NO: 442)

LCDR1: QSISSY (SEQ ID NO: 443)

LCDR2: AAS (SEQ ID NO: 444)

LCDR3: QQSYSTPPIT (SEQ ID NO: 445)

### 12848B (REGN16827 Anti-hTfR scFv:hGAA)

HCVR (V_{H}) Nucleotide Sequence

GAAGTGCAGCTGGTGGAGTCTGGGGGAGGCTTGGTACAGCCTGGCAGGTC
CCTGACACTCTCCTGTGCAGCCTCTGGATTCACCTTTGATAATTTTGGCA
TGCACTGGGTCCGGCAAGGTCCAGGGAAGGGCCTGGAATGGGTCTCAGGT
CTTACTTGGAATAGTGGTGTCATAGGCTATGCGGACTCTGTGAAGGGCCG
ATTCACCATCTCCAGAGACAACGCCAAGAACTCCCTGTATCTGCAAATGA
ACAGTCTGAGACCTGAGGACACGGCCTTATATTACTGTGCAAAAGATATA
CGGAATTACGGCCCCTTTGACTACTGGGGCCAGGGAACCCTGGTCACCGT
CTCCTCA (SEQ ID NO: 446)

HCVR (V_{H}) Amino Acid Sequence

EVQLVESGGGLVQPGRSLTLSCAASGFTFDNFGMHWVRQGPGKGLEWVSG
LTWNSGVIGYADSVKGRFTISRDNAKNSLYLQMNSLRPEDTALYYCAKDI
RNYGPFDYWGQGTLVTVSS (SEQ ID NO: 447)

HCDR1:GFTFDNFG (SEQ ID NO: 448)

HCDR2:LTWNSGVI (SEQ ID NO: 449)

HCDR3:AKDIRNYGPFDY (SEQ ID NO: 450)

LCVR (V_{L}) Nucleotide Sequence

GAAATTGTGTTGACGCAGTCTCCAGGCACCCTGTCTTTGTCTCCAGGGGA
AAGAGCCACCCTCTCCTGCAGGGCCAGTCAGAGTGTTAGCAGCAGCTACT
TAGCCTGGTACCAGCAGAAACCTGGCCAGGCTCCCAGGCTCCTCATCTAT
GGTGCATCCAGCAGGGCCACTGGCATCCCAGACAGGTTCAGTGGCAGTGG
GTCTGGGACAGACTTCACTCTCACCATCAGCAGACTGGAGCCTGAAGATT
TTGCAGTGTATTACTGTCAGCAGTATGGTAGCTCACCTTGGACGTTCGGC
CAAGGGACCAAGGTGGAAATCAAA (SEQ ID NO: 451)

LCVR (V_{L}) Amino Acid Sequence

EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPWTFG
QGTKVEIK (SEQ ID NO: 452)

LCDR1: QSVSSSY (SEQ ID NO: 453)

LCDR2: GAS (SEQ ID NO: 454)

LCDR3: QQYGSSPWT (SEQ ID NO: 455)

### 12843B (REGN17075 Anti-hTfR scFv; REGN16824 aAnti-hTfR scFv:hGAA; REGN17081 Anti-hTfR Fab)

HCVR (V_{H}) Nucleotide Sequence

GAGGTGCAGCTGGTGGAGTCTGGGGGAGGCTTAGTACAGCCTGGAGGGTC
CCTAAGACTCTCCTGTGCAGCCTCTGGATTCACCTTCAATATTTTTGAAA
TGAACTGGGTCCGCCAGGCTCCAGGGAAGGGGCTGGAGTGGATTTCCTAC
ATTAGTAGTCGTGGAACTACCACATACTACGCAGACTCTGTGAGGGGCCG
ATTCACCATCTCCAGAGACAACGCCAAGAACTCACTGTATCTGCAAATGA
ACAGCCTGAGAGCCGAGGACACGGCTGTTTATTACTGTGCGAGAGATTAT
GAAGCAACAATCCCTTTTGACTTCTGGGGCCAGGGAACCCTGGTCACCGT
CTCCTCA (SEQ ID NO: 456)

HCVR (V_{H}) Amino Acid Sequence

EVQLVESGGGLVQPGGSLRLSCAASGFTFNIFEMNWVRQAPGKGLEWISY
ISSRGTTTYYADSVRGRFTISRDNAKNSLYLQMNSLRAEDTAVYYCARDY
EATIPFDFWGQGTLVTVSS (SEQ ID NO: 457)

HCDR1:GFTFNIFE (SEQ ID NO: 458)

HCDR2:ISSRGTTT (SEQ ID NO: 459)

HCDR3:ARDYEATIPFDF (SEQ ID NO: 460)

LCVR (V_{L}) Nucleotide Sequence

GACATCCAGATGACCCAGTCTCCATCCTCCCTGTCTGCATCTGTAGGAGA
CAGAGTCACCATCACTTGCCGGGCAAGTCAGAGCATTAGCAGCTATTTAA
ATTGGTATCAGCAGAAACCAGGGAAAGCCCCTAAGCTCCTGATCTATGCT
GCATCCAGTTTGCAAAGTGGGGTCCCGTCAAGGTTCAGTGGCAGTGGATC
TGGGACAGATTTCACTCTCACCATCAGCAGTCTGCAACCTGAAGATTTTG
CAACTTACTACTGTCAACAGAGTTACAGTACCCCTCCGATCACCTTCGGC
CAAGGGACACGACTGGAGATTAAA (SEQ ID NO: 461)

LCVR (V_{L}) Amino Acid Sequence

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPITFG
QGTRLEIK (SEQ ID NO: 462)

LCDR1: QSISSY (SEQ ID NO: 463)

LCDR2: AAS (SEQ ID NO: 464)

LCDR3: QQSYSTPPIT (SEQ ID NO: 465)

### 12844B

HCVR (V_{H}) Nucleotide Sequence

GAGGTGCAGCTGGTGGAGTCTGGGGGAAGTGTGGTACGGCCTGGGGGGTC
CCTGAGACTCTCCTGTGAAGCCTCTGGATTCACCTTTGATGATTATGGCA
TGAGCTGGGTCCGCCAAGATCCAGGGAAGGGGCTGGAGTGGGTCTCTGGT
ATTAATTGGAATGGTGATAGAACAAATTATGCAGACTCTGTGAAGGGCCG
ATTCATCATTTCCAGAGACAACGCCAAGAACTCTGTGTATCTACAAATGA
ACAGTCTGAGAGCGGAGGACTCGGCCTTGTATCACTGTGCGAGAGATCAG
GGACTCGGAGTGGCAGCTACCCTTGACTACTGGGGCCAGGGAACCCTGGT
CACCGTCTCCTCA (SEQ ID NO: 466)

HCVR (V_{H}) Amino Acid Sequence

EVQLVESGGSVVRPGGSLRLSCEASGFTFDDYGMSWVRQDPGKGLEWVSG
INWNGDRTNYADSVKGRFIISRDNAKNSVYLQMNSLRAEDSALYHCARDQ
GLGVAATLDYWGQGTLVTVSS (SEQ ID NO: 467)

HCDR1:GFTFDDYG (SEQ ID NO: 468)

HCDR2:INWNGDRT (SEQ ID NO: 469)

HCDR3:ARDQGLGVAATLDY (SEQ ID NO: 470)

LCVR (V_{L}) Nucleotide Sequence

GACATCCAGATGACCCAGTCTCCATCCTCCCTGTCTGCATCTGTAGGAGA
CAGAGTCACCATCACTTGCCGGGCAAGTCAGAGCATTAGCAGCTATTTAA
ATTGGTATCAGCAGAAACCAGGGAAAGCCCCTAAGCTCCTGATCTATGCT
GCATCCAGTTTGCAAAGTGGGGTCCCGTCAAGGTTCAGTGGCAGTGGATC
TGGGACAGATTTCACTCTCACCATCAGCAGTCTGCAACCTGAAGATTTTG
CAACTTACTACTGTCAACAGAGTTACAGTACCCCTCCGATCACCTTCGGC
CAAGGGACACGACTGGAGATTAAA (SEQ ID NO: 471)

LCVR (V_{L}) Amino Acid Sequence

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPITFG
QGTRLEIK (SEQ ID NO: 472)

LCDR1: QSISSY (SEQ ID NO: 473)

LCDR2: AAS (SEQ ID NO: 474)

LCDR3: QQSYSTPPIT (SEQ ID NO: 475)

### 12845B (REGN17082 Fab; REGN17076 scFv; REGN16825 Anti-hTfR scFv:hGAA)

HCVR (V_{H}) Nucleotide Sequence

GAGGTGCAGCTGGTGGAGTCTGGGGGAGGCTTGGTACAGCCTGGAGGGTC
CCTGAGACTCTCCTGTGCAGCCTCTGGATTCACCGTCAGTAATTATGAAA
TGAACTGGGTCCGCCAGGCTCCAGGGAAGGGGCTGGAGTGGGTTTCATAC
ATTAGTAGTAGTACCAGTAACATATACTACGCAGACTCTGTGAAGGGCCG
ATTCACCATCTCCAGAGACAACGCCGAGAACTCACTGTATCTGCAGATGA
ACAGCCTGAGAGTCGAGGACACGGCTGTTTATTACTGTGTGAGAGATGGG
ATTGTAGTAGTTCCAGTTGGTCGTGGATACTACTATTACGGTTTGGACGT
CTGGGGCCAAGGGACCACGGTCACCGTCTCCTCA (SEQ ID NO: 476
)

HCVR (V_{H}) Amino Acid Sequence

EVQLVESGGGLVQPGGSLRLSCAASGFTVSNYEMNWVRQAPGKGLEWVSY
ISSSTSNIYYADSVKGRFTISRDNAENSLYLQMNSLRVEDTAVYYCVRDG
IVVVPVGRGYYYYGLDVWGQGTTVTVSS (SEQ ID NO: 477)

HCDR1:GFTVSNYE (SEQ ID NO: 478)

HCDR2:ISSSTSNI (SEQ ID NO: 479)

HCDR3: VRDGIVVVPVGRGYYYYGLDV (SEQ ID NO: 480)

LCVR (V_{L}) Nucleotide Sequence

GACATCCAGATGACCCAGTCTCCATCCTCCCTGTCTGCATCTGTAGGAGA
CAGAGTCACCATCACTTGCCGGGCAAGTCAGAGCATTAGCAGCTATTTAA
ATTGGTATCAGCAGAAACCAGGGAAAGCCCCTAAGCTCCTGATCTATGCT
GCATCCAGTTTGCAAAGTGGGGTCCCGTCAAGGTTCAGTGGCAGTGGATC
TGGGACAGATTTCACTCTCACCATCAGCAGTCTGCAACCTGAAGATTTTG
CAACTTACTACTGTCAACAGAGTTACAGTACCCCTCCGATCACCTTCGGC
CAAGGGACACGACTGGAGATTAAA (SEQ ID NO: 481)

LCVR (V_{L}) Amino Acid Sequence

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPITFG
QGTRLEIK (SEQ ID NO: 482)

LCDR1: QSISSY (SEQ ID NO: 483)

LCDR2: AAS (SEQ ID NO: 484)

LCDR3: QQSYSTPPIT (SEQ ID NO: 485)

### 12839B (REGN17080 Fab; REGN17074 scFv; REGN16822 Anti-hTfR scFv:hGAA)

HCVR (V_{H}) Nucleotide Sequence

CAGGTGCAGCTGGTGGAGTCTGGGGGAGGCGTGGTCCAGCCTGGAAGGTC
CCTGAGACTCTCCTGCGCAGCCTCTGGATTCCCCTTTAGTAATTATGTCA
TGTATTGGGTCCGCCAGGCTCCAGGCAAGGGGCTGGAGTGGGTGGCTCTT
ATTTTTTTTGACGGAAAGAAAAACTATCATGCAGACTCCGTGAAGGGCCG
ATTCACCATAACCAGAGACAATTCCAAAAATATGTTATATCTGCAAATGA
ACAGCCTGAGACCTGAGGACGCGGCTGTGTATTACTGTGCGAAAATCCAT
TGTCCTAATGGTGTATGTTACAAGGGGTATTACGGAATGGACGTCTGGGG
CCAAGGGACCACGGTCACCGTCTCCTCA (SEQ ID NO: 486)

HCVR (V_{H}) Amino Acid Sequence

QVQLVESGGGVVQPGRSLRLSCAASGFPFSNYVMYWVRQAPGKGLEWVAL
IFFDGKKNYHADSVKGRFTITRDNSKNMLYLQMNSLRPEDAAVYYCAKIH
CPNGVCYKGYYGMDVWGQGTTVTVSS (SEQ ID NO: 487)

HCDR1:GFPFSNYV (SEQ ID NO: 488)

HCDR2:IFFDGKKN (SEQ ID NO: 489)

HCDR3:AKIHCPNGVCYKGYYGMDV (SEQ ID NO: 490)

LCVR (V_{L}) Nucleotide Sequence

GACATCCAGATGACCCAGTCTCCATCCTCCCTGTCTGCATCTGTAGGAGA
CAGAGTCACCATCACTTGCCGGGCAAGTCAGAGCATTAGCAGCTATTTAA
ATTGGTATCAGCAGAAACCAGGGAAAGCCCCTAAGCTCCTGATCTATGCT
GCATCCAGTTTGCAAAGTGGGGTCCCGTCAAGGTTCAGTGGCAGTGGATC
TGGGACAGATTTCACTCTCACCATCAGCAGTCTGCAACCTGAAGATTTTG
CAACTTACTACTGTCAACAGAGTTACAGTACCCCTCCGATCACCTTCGGC
CAAGGGACACGACTGGAGATTAAA (SEQ ID NO: 491)

LCVR (V_{L}) Amino Acid Sequence

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPITFG
QGTRLEIK (SEQ ID NO: 492)

LCDR1: QSISSY (SEQ ID NO: 493)

LCDR2: AAS (SEQ ID NO: 494)

LCDR3: QQSYSTPPIT (SEQ ID NO: 495)

### 12841B (REGN16823 Anti-hTfR scFv:hGAA)

HCVR (V_{H}) Nucleotide Sequence

GAGGTGCAGCTGGTGGAGTCTGGGGGAGGCTTGGTCCAGCCTGGGGGGTC
CCTAAGACTCTCCTGTGCAGCCTCTGGATTCACCTTTAGTAACTATTGGA
TGAACTGGGTCCGCCAGGCTCCAGGGAAGGGACTGGAGTGGGTGGCCAAT
ATAAAAGAAGATGGAGGTAAGAAATTGTATGTGGACTCTGTGAAGGGCCG
ATTCACCATCTCCAGAGACAACGCCAAGAACTCACTGTTTCTGCAAATGA
ACAGCCTGAGAGCCGAGGACACGGCTGTGTATTATTGTGCGAGAGAAGAT
ACAACTTTGGTTGTGGACTACTACTACTACGGTATGGACGTCTGGGGCCA
AGGGACCACGGTCACCGTCTCCTCA (SEQ ID NO: 496)

HCVR (V_{H}) Amino Acid Sequence

EVQLVESGGGLVQPGGSLRLSCAASGFTFSNYWMNWVRQAPGKGLEWVAN
IKEDGGKKLYVDSVKGRFTISRDNAKNSLFLQMNSLRAEDTAVYYCARED
TTLVVDYYYYGMDVWGQGTTVTVSS (SEQ ID NO: 497)

HCDR1:GFTFSNYW (SEQ ID NO: 498)

HCDR2:IKEDGGKK (SEQ ID NO: 499)

HCDR3:AREDTTLVVDYYYYGMDV (SEQ ID NO: 500)

LCVR (V_{L}) Nucleotide Sequence

GACATCCAGATGACCCAGTCTCCATCCTCCCTGTCTGCATCTGTAGGAGA
CAGAGTCACCATCACTTGCCGGGCAAGTCAGAGCATTAGCAGCTATTTAA
ATTGGTATCAGCAGAAACCAGGGAAAGCCCCTAAGCTCCTGATCTATGCT
GCATCCAGTTTGCAAAGTGGGGTCCCGTCAAGGTTCAGTGGCAGTGGATC
TGGGACAGATTTCACTCTCACCATCAGCAGTCTGCAACCTGAAGATTTTG
CAACTTACTACTGTCAACAGAGTTACAGTACCCCTCCGATCACCTTCGGC
CAAGGGACACGACTGGAGATTAAA (SEQ ID NO: 501)

LCVR (V_{L}) Amino Acid Sequence

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPITFG
QGTRLEIK (SEQ ID NO: 502)

LCDR1: QSISSY (SEQ ID NO: 503)

LCDR2: AAS (SEQ ID NO: 504)

LCDR3: QQSYSTPPIT (SEQ ID NO: 505)

### 12850B (REGN16828 Anti-hTfR scFv:hGAA)

HCVR (V_{H}) Nucleotide Sequence

CAGGTCCAGCTGGTGCAGTCTGGGGCTGAGGTGAAGAAGCCTGGGTCCTC
GGTGAAGGTCTCCTGCAAGGCTTCTGGAGGCACCTTCAACACCTATGCTA
TCACCTGGGTGCGACAGGCCCCTGGACAAGGGCTTGAATGGATGGGGGGA
ATCATCCCTATCTCTGGCATAGCAGAGTACGCACAGAAGTTCCAGGGCAG
AGTCACGATCACCACGGATGACTCCTCGACCACAGCCTACATGGAACTGA
ACAGTCTGAGATCTGAGGACACGGCCGTGTATTACTGTGCGAGCTGGAAC
TACGCACTCTACTACTTCTACGGTATGGACGTCTGGGGCCGAGGGACCAC
GGTCACCGTCTCCTCA (SEQ ID NO: 506)

HCVR (V_{H}) Amino Acid Sequence

QVQLVQSGAEVKKPGSSVKVSCKASGGTFNTYAITWVRQAPGQGLEWMGG
IIPISGIAEYAQKFQGRVTITTDDSSTTAYMELNSLRSEDTAVYYCASWN
YALYYFYGMDVWGRGTTVTVSS (SEQ ID NO: 507)

HCDR1:GGTFNTYA (SEQ ID NO: 508)

HCDR2:IIPISGIA (SEQ ID NO: 509)

HCDR3:ASWNYALYYFYGMDV (SEQ ID NO: 510)

LCVR (V_{L}) Nucleotide Sequence

GAAATTGTGTTGACGCAGTCTCCAGGCACCCTGTCTTTGTCTCCAGGGGA
AAGAGCCACCCTCTCCTGCAGGGCCAGTCAGAGTGTTAGCAGCAGCTACT
TAGCCTGGTACCAGCAGAAACCTGGCCAGGCTCCCAGGCTCCTCATCTAT
GGTGCATCCAGCAGGGCCACTGGCATCCCAGACAGGTTCAGTGGCAGTGG
GTCTGGGACAGACTTCACTCTCACCATCAGCAGACTGGAGCCTGAAGATT
TTGCAGTGTATTACTGTCAGCAGTATGGTAGCTCACCTTGGACGTTCGGC
CAAGGGACCAAGGTGGAAATCAAA (SEQ ID NO: 511)

LCVR (V_{L}) Amino Acid Sequence

EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPWTFG
QGTKVEIK (SEQ ID NO: 512)

LCDR1: QSVSSSY (SEQ ID NO: 513)

LCDR2: GAS (SEQ ID NO: 514)

LCDR3: QQYGSSPWT (SEQ ID NO: 515)

### 69261

HCVR (V_{H}) Nucleotide Sequence

CAGGTGCAGCTGGTGGAGTCTGGGGGAGGCTTGGTCAAGCCTGGAGGGTC
CCTGAGACTCTCCTGTGCAGCCTCTGGATTCACCTTCAGTGTCTATTACA
TGAACTGGATCCGCCAGGCTCCAGGGAAGGGCCTGGAGTGGGTTTCATAC
ATTAGTAGTAGTGGTAGTACCATATACTACGCAGACTCTGTGAAGGGCCG
ATTCACCATCTCCAGGGACAACGCCAAGAACTCACTGTATCTCCAAATGA
ACAGTCTGAGAGCCGAGGACACGGCCGTATATTACTGTGGGAGAGAAGGG
TATAGTGGGACTTATTCTTATTACGGTATGGACGTCTGGGGCCAAGGGAC
CACGGTCACCGTCTCCTCA (SEQ ID NO: 516)

HCVR (V_{H}) Amino Acid Sequence

QVQLVESGGGLVKPGGSLRLSCAASGFTFSVYYMNWIRQAPGKGLEWVSY
ISSSGSTIYYADSVKGRFTISRDNAKNSLYLQMNSLRAEDTAVYYCGREG
YSGTYSYYGMDVWGQGTTVTVSS (SEQ ID NO: 517)

HCDR1:GFTFSVYY (SEQ ID NO: 518)

HCDR2:ISSSGSTI (SEQ ID NO: 519)

HCDR3:GREGYSGTYSYYGMDV (SEQ ID NO: 520)

LCVR (V_{L}) Nucleotide Sequence

GATATTGTGATGACTCAGTCTCCACTCTCCCTGCCCGTCACCCCTGGAGA
GCCGGCCTCCATCTCCTGCAGGTCTAGTCAGAGCCTCCTGCATAGTAATG
GATACAACTATTTGGATTGGTACCTGCAGAAGCCAGGGCAGTCTCCACAG
TTCCTGATCTATTTGGGTTCTAATCGGGCCTCCGGGGTCCCTGACAGGTT
CAGTGGCAGTGGATCAGGCACAGATTTTACACTGAAAATCAACAGAGTGG
AGGCTGAGGATGTTGGGGTTTATTACTGCATGCAAGCTCTACAAACTCCG
TACACTTTTGGCCAGGGGACCAAGCTGGAGATCAAA (SEQ IDNO: 52
1)

LCVR (V_{L}) Amino Acid Sequence

DIVMTQSPLSLPVTPGEPASISCRSSQSLLHSNGYNYLDWYLQKPGQSPQ
FLIYLGSNRASGVPDRFSGSGSGTDFTLKINRVEAEDVGVYYCMQALQTP
YTFGQGTKLEIK (SEQ ID NO: 522)

LCDR1: QSLLHSNGYNY (SEQ ID NO: 523)

LCDR2: LGS (SEQ ID NO: 524)

LCDR3: MQALQTPYT (SEQ ID NO: 525)

### 69263

HCVR (V_{H}) Nucleotide Sequence

GAAGTGCAGCTGGTGGAGTCTGGGGGAGGGTTGGTACAGCCTGGCAGGTC
CCTGAGACTCTCCTGTGCAGTCTCTGGATTCACCTTTGATGATTATGCCA
TGCACTGGGTCCGGCAAGCTCCAGGGAAGGGCCTGGAGTGGGTCTCAGGT
ATTAGTTGGAATAGTGGTACCAGAGGATATGCGGACTCTGTGAAGGGCCG
ATTCACCATCTCCAGAGACAACGCCAAGAACTCCCTGTATCTGCAAATGA
ACAGTCTGAGAGGTGAGGACACGGCCTTGTATTACTGTGTAAAAGATATT
ACGATATCCCCCAACTACTACGGTATGGACGTCTGGGGCCAAGGGACCAC
GGTCACCGTCTCCTCA (SEQ ID NO: 526)

HCVR (V_{H}) Amino Acid Sequence

EVQLVESGGGLVQPGRSLRLSCAVSGFTFDDYAMHWVRQAPGKGLEWVSG
ISWNSGTRGYADSVKGRFTISRDNAKNSLYLQMNSLRGEDTALYYCVKDI
TISPNYYGMDVWGQGTTVTVSS (SEQ ID NO: 527)

HCDR1:GFTFDDYA (SEQ ID NO: 528)

HCDR2:ISWNSGTR (SEQ ID NO: 529)

HCDR3: VKDITISPNYYGMDV (SEQ ID NO: 530)

LCVR (V_{L}) Nucleotide Sequence

GACATCCAGATGACCCAGTCTCCATCCTCCCTGTCTGCATCTGTAGGAGA
CAGAGTCACCATCACTTGCCGGGCGAGTCAGGACATTAGCCATTATTCAG
CCTGGTATCAGCAGAAACCAGGGAAACTTCCTAACCTCCTGATCTATGCT
GCATCCACTTTGCAATCAGGGGTCCCATCTCGGTTCAGTGGCAGTGGATC
TGGGACAGATTTCTCTCTCACCACCAGCAGCCTGCAGCCTGAAGATGTTG
CAACTTATTACTGTCAAAAGTATAACAGTGTCCCTCTCACTTTCGGCGGA
GGGACCAAGGTGGAGATCAAA (SEQ ID NO: 531)

LCVR (V_{L}) Amino Acid Sequence

DIQMTQSPSSLSASVGDRVTITCRASQDISHYSAWYQQKPGKLPNLLIYA
ASTLQSGVPSRFSGSGSGTDFSLTTSSLQPEDVATYYCQKYNSVPLTFGG
GTKVEIK (SEQ ID NO: 532)

LCDR1: QDISHY (SEQ ID NO: 533)

LCDR2: AAS (SEQ ID NO: 534)

LCDR3: QKYNSVPLT (SEQ ID NO: 535)

TABLE 30

Anti-hTfR scFv Molecules in Fusion Proteins

| Antibody clone | SEQ ID NO | Amino acid sequence (Vk-3xG4S-Vh) |
|---|---|---|
| 12795B | 538 | DIQMTQSPSSLSASVGDRVTITCRASQGIRDHFGWYQQKPGKAPKRLIYAASSLHSGVPSRFS GSGSGTEFTLTISSLQPEDFATYYCLQYDTYPLTFGGGTKVEIKGGGGSGGGGSGGGGSEVQ LVESGGGLVQPGGSLRLSCATSGFTFTSYDMKWVRQAPGLGLEWVSAISGSGGNTYYADSV KGRFTISRDNSRNTLYLQMNSLRAEDTAVYYCTRSHDFGAFDYFDYWGQGTLVTVSS |
| 12798B | 539 | EIVMTQSPATLSVSPGERATLSCRASQTVSSNLAWYQQKPGQAPRLLIYGSSSRATGIPARFS GSGSGTEFTLTISSLQSEDFAVYYCQQYNNWPPYTFGQGTKLEIKGGGGSGGGGSGGGGSEV QLVESGGDLVQPGRSLRLSCAASGFTFDDYAMHWVRQAPGKGLEWVSGISWNSATRVYAD SVKGRFTISRDNAKNFLYLQMNSLRSEDTALYHCAKDMDISLGYYGLDVWGQGTTVTVSS |
| 12799B | 540 | DIQMTQSPSSVSASVGDRVTITCRASQGIASWLAWYQQKPGKAPELLIYAASSLQGGVPSRF SGSGSGTDFTLTISSLQPEDFAIYYCQQANYFPWTFGQGTKVEIKGGGGSGGGGSGGGGSQIT LKESGPTLVKPTQTLTLTCTFSGFSLSTSGVGVVWIRQPPGKALEWLALIYWNDHKRYSPSL GSRLTITKDTSKNQVVLTMTNMDPVDTATYYCAHYSGSYSYYYYGLDVWGQGTTVTVSS |
| 12801B | 541 | DIQMTQSPSSLSASVGDRVTITCRASQGIRTDLGWYQQKPGKAPKRLIYAASSLQSGVPSRFS GSGSGTEFTLTISSLRPEDFATFYCLQYNSYPLTFGGGTKVEIKGGGGSGGGGSGGGGSEVQL LESGGALVQPGGSLRLSCAASGFTFTSYAMHWVRQAPGKGLEWVSSIRGSGGGTYSADSVK GRFTISRDNSRDTLYLQMNSVRAEDTAVYYCARSHDYGAFDFFDYWGQGTLVTVSS |
| 12802B | 542 | EIVMTQSPATLSVSPGERATLSCRASQSVSINLAWYQQKPGQAPRLLIFVASTRATGIPARFSG SGSGTEFTLTISSLQSEDFATYYCQQYDIWPYTFGQGTKLEIKGGGGSGGGGSGGGGSQVQL VESGGGLVKPGGSLRLSCAASGFTFSDYFMSWIRQAPGKGLEWVSYISSTGSTINYADSVKG RFTISRDNVKNSLYLQMTSLRVEDTAVYYCTRDNWNYEYWGQGTLVTVSS |
| 12808B | 543 | DIQMTQSPSSLSASVGDRVTINCRASQGIRNDLGWYQQKPGKAPKRLIYAASSLQSGVPLRFS GSGSGTEFTLTINNLQPEDFATYYCLSHNSYPWTFGQGTKVEfKGGGGSGGGGSGGGGSQLQ LQESGPGLVKPSETLSLTCTVSGESISSNTYYWGWIRQPPGKGLEWIGSIDYSGTTNYNPSLK SRVTISVDTSRNHFSLRLRSVTAADTAVYYCAREWGNYGYYYGMDVWGQGTTVTVSS |
| 12812B | 544 | DIQMTQSPPSVSASVGDRVTITCRASQGISSWLAWYQQKPGKAPKLLIYAASSLQSGVPSRFS GSGSGTDFTLTISSLQPEDFATYYCQQANSFPRTFGQGTKVEIKGGGGSGGGGSGGGGSQVQ LVQSGAEVKKPGSSVRVSCKASRGTFSSYAISWVRQAPGQGLEWMGGIIPIFGTANYAQKFL ARVTITADESTSTAYMELSSLRSEDTAVYYCAREKGWNYFDYWGQGTLVTVSS |
| 12816B | 545 | DIVMTQSPLSLPVTPGEPASISCRSSQSLLHGNGYNYLTWYLQKPGQSPQLLIYLGSNRASGV PDRFSGSGSGTDFTLKISRVEAEDVGVYYCMQALQTPYTFGQGTKLEIKGGGGSGGGGSGG GGSQVQLVESGGGLVKPGGSLRLSCAASGFTFSDYYMNWIRQAPGKGLEWVSYISSSGTTIY YADSVKGRFTISRDNAKKSLYLEMNSLRAEDTAVYYCAREGYGNDYYYYGIDVWGQGTTV TVSS |
| 12833B | 546 | DIQMTQSPSSLSASVGDRVTITCRASQSISSYLNWYQQKPGKAPKLLIYAASSLQSGVPSRFS GSGSGTDFTLTISSLQPEDFATYYCQQSYSTPPITFGQGTRLEIKGGGGSGGGGSGGGGSQVQ LVESGGGVVQPGRSLRLSCAASGFTFSSFGMHWVRQAPGKGLEWVIFISYDGSDKYYADSV KGRFAISRDSSKNTLYLQMNSLRAEDTAVYYCAKENGILTDSYGMDVWGQGTTVTVSS |
| 12834B | 547 | DIQMTQSPSSLSASVGDRVTITCRASQSISSYLNWYQQKPGKAPKLLIYAASSLQSGVPSRFS GSGSGTDFTLTISSLQPEDFATYYCQQSYSTPPITFGQGTRLEIKGGGGSGGGGSGGGGSQVQ LVQSGAEVKKPGASVKVSCKASGYTFTSYGISWVRQAPGQGLEWMGWISVYHGNTNYAQ KFQGRVTMTTDTSTSTAYMELRSLRSDDTAVYYCAREGYYDFWSGYYPFDYWGQGTLVT VSS |
| 12835B | 548 | DIQMTQSPSSLSASVGDRVTITCRASQSISSYLNWYQQKPGKAPKLLIYAASSLQSGVPSRFS GSGSGTDFTLTISSLQPEDFATYYCQQSYSTPPITFGQGTRLEIKGGGGSGGGGSGGGGSEVQ LVESGGGLIQPGGSLRLSCEASGFTFRNYEMNWVRQAPGKGLEWVSYISSSGNMKDYAESV KGRFTISRDNVKNSLQLQMNSLRVEDTAVYYCARDEFPYGMDVWGQGTTVTVSS |
| 12839B | 549 | DIQMTQSPSSLSASVGDRVTITCRASQSISSYLNWYQQKPGKAPKLLIYAASSLQSGVPSRFS GSGSGTDFTLTISSLQPEDFATYYCQQSYSTPPITFGQGTRLEIKGGGGSGGGGSGGGGSQVQ LVESGGGVVQPGRSLRLSCAASGFPFSNYVMYWVRQAPGKGLEWVALIFFDGKKNYHADS VKGRFTITRDNSKNMLYLQMNSLRPEDAAVYYCAKIHCPNGVCYKGYYGMDVWGQGTTV TVSS |
| 12841B | 550 | DIQMTQSPSSLSASVGDRVTITCRASQSISSYLNWYQQKPGKAPKLLIYAASSLQSGVPSRFS GSGSGTDFTLTISSLQPEDFATYYCQQSYSTPPITFGQGTRLEIKGGGGSGGGGSGGGGSEVQ LVESGGGLVQPGGSLRLSCAASGFTFSNYWMNWVRQAPGKGLEWVANIKEDGGKKLYVD SVKGRFTISRDNAKNSLFLQMNSLRAEDTAVYYCAREDTTLVVDYYYYGMDVWGQGTTV TVSS |
| 12843B | 551 | DIQMTQSPSSLSASVGDRVTITCRASQSISSYLNWYQQKPGKAPKLLIYAASSLQSGVPSRFS GSGSGTDFTLTISSLQPEDFATYYCQQSYSTPPITFGQGTRLEIKGGGGSGGGGSGGGGSEVQ LVESGGGLVQPGGSLRLSCAASGFTFNIFEMNWVRQAPGKGLEWISYISSRGTTTYYADSVR GRFTISRDNAKNSLYLQMNSLRAEDTAVYYCARDYEATIPFDFWGQGTLVTVSS |
| 12844B | 552 | DIQMTQSPSSLSASVGDRVTITCRASQSISSYLNWYQQKPGKAPKLLIYAASSLQSGVPSRFS GSGSGTDFTLTISSLQPEDFATYYCQQSYSTPPITFGQGTRLEIKGGGGSGGGGSGGGGSEVQ LVESGGSVVRPGGSLRLSCEASGFTFDDYGMSWVRQDPGKGLEWVSGINWNGDRTNYADS VKGRFIISRDNAKNSVYLQMNSLRAEDSALYHCARDQGLGVAATLDYWGQGTLVTVSS |
| 12845B | 553 | DIQMTQSPSSLSASVGDRVTITCRASQSISSYLNWYQQKPGKAPKLLIYAASSLQSGVPSRFS GSGSGTDFTLTISSLQPEDFATYYCQQSYSTPPITFGQGTRLEIKGGGGSGGGGSGGGGSEVQ LVESGGGLVQPGGSLRLSCAASGFTVSNYEMNWVRQAPGKGLEWVSYISSSTSNIYYADSV KGRFTISRDNAENSLYLQMNSLRVEDTAVYYCVRDGIVVVPVGRGYYYYGLDVWGQGTTV TVSS |
| 12847B | 554 | DIQMTQSPSSLSASVGDRVTITCRASQSISSYLNWYQQKPGKAPKLLIYAASSLQSGVPSRFS GSGSGTDFTLTISSLQPEDFATYYCQQSYSTPPITFGQGTRLEIKGGGGSGGGGSGGGGSEVQ LVESGGGLVQPGRSLRLSCAASGFTFDDYAMNWVRQAPGKGLEWVSGISWSSGSMDYADS VKGRFTISRDNAKNSLYLQMNSLRTEDTALYYCAKAREVGDYYGMDVWGQGTTVTVSS |
| 12848B | 555 | EIVLTQSPGTLSLSPGERATLSCRASQSVSSSYLAWYQQKPGQAPRLLIYGASSRATGIPDRFS GSGSGTDFTL TISRLEPEDF A VYYCQQYGSSPWTFGQGTKVEIKGGGGSGGGGSGGGGSEVQ LVESGGGLVQPGRSLTLSCAASGFTFDNFGMHWVRQGPGKGLEWVSGLTWNSGVIGYADS VKGRFTISRDNAKNSLYLQMNSLRPEDTALYYCAKDIRNYGPFDYWGQGTLVTVSS |
| 12850B | 556 | EIVLTQSPGTLSLSPGERATLSCRASQSVSSSYLAWYQQKPGQAPRLLIYGASSRATGIPDRFS GSGSGTDFTLTISRLEPEDFAVYYCQQYGSSPWTFGQGTKVEIKGGGGSGGGGSGGGGSQV QLVQSGAEVKKPGSSVKVSCKASGGTFNTYAITWVRQAPGQGLEWMGGIIPISGIAEYAQKF QGRVTITTDDSSTTAYMELNSLRSEDTAVYYCASWNYALYYFYGMDVWGRGTTVTVSS |
| 31863B | 557 | DIQMTQSPSSLSASIGDRVTITCRASQGISNYLAWYQQKPGKVPKLLIYAASTLQSGVPSRFS GSGSGTDFTLTISSLQPEDVATYYCQNHNSVPLTFGGGTKVEIKGGGGSGGGGSGGGGSEVQ LVESGGGLVQPGGSLRLSCAASGFTFNSYAMTWVRQAPGKGLEWVSFIGGSTGNTYYAGSV KGRFTISSDNSKKTLYLQMNSLRAEDTAVYYCAKGGAARRMEYFQHWGQGTLVTVSS |
| 31874B | 558 | DIQMTQSPSSLSASVGDRVTITCRASQGISNYLAWYQQKPGKVPNLLIYAASTLQSGVPSRFS GSGSGTDFTLTISSLQPEDVATYYCQKYNSAPLTFGGGTKVEIKGGGGSGGGGSGGGGSEVQ LVESGGGLVQPGGSLRLSCAASGFAFSSYAMTWVRQAPGKGLEWVSVISGTGGSTYYADSV KGRFTISRDNSKNTLYLQMNSLRAEDTAVYYCAKGGAARRMEYFQYWGQGTLVTVSS |
| 69261 | 559 | DIVMTQSPLSLPVTPGEPASISCRSSQSLLHSNGYNYLDWYLQKPGQSPQFLIYLGSNRASGV PDRFSGSGSGTDFTLKINRVEAEDVGVYYCMQALQTPYTFGQGTKLEIKGGGGSGGGGSGG GGSQVQLVESGGGLVKPGGSLRLSCAASGFTFSVYYMNWIRQAPGKGLEWVSYISSSGSTIY YADSVKGRFTISRDNAKNSLYLQMNSLRAEDTAVYYCGREGYSGTYSYYGMDVWGQGTT VTVSS |
| 69263 | 560 | DIQMTQSPSSLSASVGDRVTITCRASQDISHYSAWYQQKPGKLPNLLIYAASTLQSGVPSRFS GSGSGTDFSLTTSSLQPEDVATYYCQKYNSVPLTFGGGTKVEIKGGGGSGGGGSGGGGSEV QLVESGGGLVQPGRSLRLSCAVSGFTFDDYAMHWVRQAPGKGLEWVSGISWNSGTRGYAD SVKGRFTISRDNAKNSLYLQMNSLRGEDTALYYCVKDITISPNYYGMDVWGQGTTVTVSS |
| 69305 | 561 | DIQMTQSPSSLSASVGDRVTITCRASQSIDRYLNWYRQKPGKAPKLLIYTTSSLQSGVPSRFS GSGSGTDFTLTLSSLQPEDFATYYCQQSYSPPLTFGGGTKVEIKGGGGSGGGGSGGGGSQVQ LVESGGGVVQPGRSLRLSCAASGFTFSSYGMHWVRQAPGKGLEWVAVIWYDGSNKYYAD SVKGRFTISRDISKNTLYLQMNSLRAEDTAVYYCAGQLDLFFDYWGQGTLVTVSS |
| 69307 | 562 | DIQMTQSPSSVSASVGDRVTITCRASQGISSWLAWYQQKPGKAPKLLIYAASSLQSGVPSRFS GSGSGTDFTLTISSLQPEDFATYYCQKADSLPYAFGQGTKLEIKGGGGSGGGGSGGGGSEVQ LVESGGGLVQPGGSLRLSCTASGFTFSNYWMTWVRQAPGKGLEWVANIKEDGSEKEYVDS VKGRFTISRDNAKNSLYLQMNSLRGEDTAVYYCARDGEQLVDYYYYYVMDVWGQGTTVT VSS |
| 69323 | 563 | DIQMTQSPSSLSASVGDRVTITCRASQSISSYLNWYQQKPGKAPKVLIYAASSLQSGVPSRFS GSGSGTDFTLTISSLQPEDFATYYCQQSYSIPLTFGGGTKVEIKGGGGSGGGGSGGGGSEVQL VESGGGLVQPGRSLRLSCAASGFTFDDYAMHWVRQAPGKGLEWVSGISWNSGYIGYADSV KGRFTISRDNAENSLHLQMNSLRAEDTALYYCARGGSTLVRGVKGGYYGMDVWGQGTTV TVSS |
| 69326 | 564 | EIVMTQSPATLSVSPGERATLSCRASQSVSSNFAWYQQKPGQAPRLLIYSASSRATGIPVRFS GSGSGTEFTLTISSLQSEDFAVYYCQQYNIWPRTFGQGTKVEIKGGGGSGGGGSGGGGSEVQ LVESGGGLVQPGGSLRLSCAVSGFIFSSYEMNWVRQAPGKGLEWVSYISSSGSTIFYADSVK GRFTISRDNAKNSLYLOMNSLRAEDTAVYYCVSGVVLFDVWGQGTMVTVSS |
| 69329 | 565 | DIQMTQSPSSVSASVGDRVTITCRASQGISSWLAWYQQKPGKAPKLLIYAASSLQSGVPSRFS GSGSGTDFTLTISSLQPEDFATYYCQKANSFPYTFGQGTKLEIKGGGGSGGGGSGGGGSEVQ LVESGGGLVQPGGSLRLSCAASGFTFSNYWMTWVRQAPGKGLEWVANIKEDGSEKDYVDS VKGRFTISRDNAKNSLYLQMNSLRGEDTAVYYCARDGEQLVDYYYYYVMDVWGQGTTVT VSS |
| 69331 | 566 | DIQLTQSPSSLSASVGDRVTITCWASQGISSYLAWYQQKPGKAPKLLIYAASTLQSGVPSRFS GSGSGTEFTLTISSLQPEDFATYYCQQLNSYPLTFGGGTKVEIKGGGGSGGGGSGGGGSQVQ LVESGGGVVQPGRSLRLSCIASGFTFSVYGIHWVRQAPGKGLEWMAVISHDGNIKHYADSV KGRFTISRDNSKNTLYLQINSLRTEDTAVYYCAKDTWNSLDTFDIWGQGTMVTVSS |
| 69332 | 567 | AIQMTQSPSSLSASVGDRVTITCRASQGIRNDLGWYQQKPGKAPKLLIYAASTLQSGVPSRFS GSGSGTDFTLTISSLQPEDFATYYCLQDYNYPFTFGPGTKVDIKGGGGSGGGGSGGGGSQVT LRESGPALVKPSQTLTLTCTFSGFSLNTYGMFVSWIRQPPGKALEWLAHIHWDDDKYYSTSL KTRLTISKDTSKNQVVLTMTNMDPVDTATYYCARGHNNLNYIIHWGQGTLVTVSS |
| 69340 | 568 | EIVLTQSPATLSLSPGERATLSCRASQSVSSYLAWYQQKPGQAPRLLIHDVSNRATGIPARFS GSGSGTDFTLTISSLEPEDFVVYYCQQRSDWPITFGQGTRLEIKGGGGSGGGGSGGGGSEVQ LVESGGGLVQPGRSLRLSCAASGFTFDDKAMHWVRQVPGKGLEWISGISWNSGTIGYADSV KGRFIISRDNAKNSLYLQMNSLRAEDTALYYCAKDGDTSGWYWYGLDVWGQGTTVTVSS |
| 69348 | 569 | DIQMTQSPSSLSASVGDRVTITCRASQSIRNVLGWFQQKPGKAPQRLIYAASSLQSGVPSRFS GSGSGTEFTLTISSLQPEDFATYYCLQHNFYPLTFGGGTKVEIKGGGGSGGGGSGGGGSQVQ LVESGGGVVQPGRSLRLSCAASGFTFTTYGMHWVRQAPGKGLEWVAVIWYDGSNKYYGD SVKGRFTISRDNSKNTLYLQMNSLRVDDTAVYYCTRTHGYTRSSDGFDYWGQGTLVTVSS |

### Heavy and Light Chains of Anti-hTfR Fabs in Anti-hTfRGAA Fusion Proteins

### 31874B

Fab Light Chain

DIQMTQSPSSLSASVGDRVTITCRASQGISNYLAWYQQKPGKVPNLLIYA
ASTLQSGVPSRFSGSGSGTDFTLTISSLQPEDVATYYCQKYNSAPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC(SEQ ID NO: 603)

Fab Heavy Chain

EVQLVESGGGLVQPGGSLRLSCAASGFAFSSYAMTWVRQAPGKGLEWVSV
ISGTGGSTYYADSVKGRFTISRDNSKNTLYLQMNSLRAEDTAVYYCAKGG
AARRMEYFQYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTH (SEQ ID NO: 604)

### 31863B

Fab Light Chain

DIQMTQSPSSLSASIGDRVTITCRASQGISNYLAWYQQKPGKVPKLLIYA
ASTLQSGVPSRFSGSGSGTDFTLTISSLQPEDVATYYCQNHNSVPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC(SEQ ID NO: 605)

Fab Heavy Chain

EVQLVESGGGLVQPGGSLRLSCAASGFTFNSYAMTWVRQAPGKGLEWVSF
IGGSTGNTYYAGSVKGRFTISSDNSKKTLYLQMNSLRAEDTAVYYCAKGG
AARRMEYFQHWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTH (SEQ ID NO: 606)

### 69348

Fab Light Chain

DIQMTQSPSSLSASVGDRVTITCRASQSIRNVLGWFQQKPGKAPQRLIYA
ASSLQSGVPSRFSGSGSGTEFTLTISSLQPEDFATYYCLQHNFYPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC(SEQ ID NO: 607)

Fab Heavy Chain

QVQLVESGGGVVQPGRSLRLSCAASGFTFTTYGMHWVRQAPGKGLEWVAV
IWYDGSNKYYGDSVKGRFTISRDNSKNTLYLQMNSLRVDDTAVYYCTRTH
GYTRSSDGFDYWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTH (SEQ ID NO: 608)

### 69340

Fab Light Chain

EIVLTQSPATLSLSPGERATLSCRASQSVSSYLAWYQQKPGQAPRLLIHD
VSNRATGIPARFSGSGSGTDFTLTISSLEPEDFVVYYCQQRSDWPITFGQ
GTRLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC(SEQ ID NO: 609)

Fab Heavy Chain

EVQLVESGGGLVQPGRSLRLSCAASGFTFDDKAMHWVRQVPGKGLEWISG
ISWNSGTIGYADSVKGRFIISRDNAKNSLYLQMNSLRAEDTALYYCAKDG
DTSGWYWYGLDVWGQGTTVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTH (SEQ ID NO: 610)

### 69331

Fab Light Chain

DIQLTQSPSSLSASVGDRVTITCWASQGISSYLAWYQQKPGKAPKLLIYA
ASTLQSGVPSRFSGSGSGTEFTLTISSLQPEDFATYYCQQLNSYPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC(SEQ ID NO: 611)

Fab Heavy Chain

QVQLVESGGGVVQPGRSLRLSCIASGFTFSVYGIHWVRQAPGKGLEWMAV
ISHDGNIKHYADSVKGRFTISRDNSKNTLYLQINSLRTEDTAVYYCAKDT
WNSLDTFDIWGQGTMVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTH (SEQ ID NO: 612)

### 69332

Fab Light Chain

AIQMTQSPSSLSASVGDRVTITCRASQGIRNDLGWYQQKPGKAPKLLIYA
ASTLQSGVPSRFSGSGSGTDFTLTISSLQPEDFATYYCLQDYNYPFTFGP
GTKVDIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC(SEQ ID NO: 613)

Fab Heavy Chain

QVTLRESGPALVKPSQTLTLTCTFSGFSLNTYGMFVSWIRQPPGKALEWL
AHIHWDDDKYYSTSLKTRLTISKDTSKNQVVLTMTNMDPVDTATYYCARG
HNNLNYIIHWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTH (SEQ ID NO: 614)

### 69326

Fab Light Chain

EIVMTQSPATLSVSPGERATLSCRASQSVSSNFAWYQQKPGQAPRLLIYS
ASSRATGIPVRFSGSGSGTEFTLTISSLQSEDFAVYYCQQYNIWPRTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC(SEQ ID NO: 615)

Fab Heavy Chain

EVQLVESGGGLVQPGGSLRLSCAVSGFIFSSYEMNWVRQAPGKGLEWVSY
ISSSGSTIFYADSVKGRFTISRDNAKNSLYLQMNSLRAEDTAVYYCVSGV
VLFDVWGQGTMVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTH (SEQ ID NO: 616)

### 69329

Fab Light Chain

DIQMTQSPSSVSASVGDRVTITCRASQGISSWLAWYQQKPGKAPKLLIYA
ASSLQSGVPSRFSGSGSGTDFTLTISSLQPEDFATYYCQKANSFPYTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC(SEQ ID NO: 617)

Fab Heavy Chain

EVQLVESGGGLVQPGGSLRLSCAASGFTFSNYWMTWVRQAPGKGLEWVAN
IKEDGSEKDYVDSVKGRFTISRDNAKNSLYLQMNSLRGEDTAVYYCARDG
EQLVDYYYYYVMDVWGQGTTVTVSSASTKGPSVFPLAPSSKSTSGGTAAL
GCLVKDYFPEPVTVSWNSGALTSGVHTFPAVLQSSGLYSLSSVVTVPSSS
LGTQTYICNVNHKPSNTKVDKKVEPKSCDKTH (SEQ ID NO: 618)

### 69323

Fab Light Chain

DIQMTQSPSSLSASVGDRVTITCRASQSISSYLNWYQQKPGKAPKVLIYA
ASSLQSGVPSRFSGSGSGTDFTLTISSLQPEDFATYYCQQSYSIPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC(SEQ ID NO: 619)

Fab Heavy Chain

EVQLVESGGGLVQPGRSLRLSCAASGFTFDDYAMHWVRQAPGKGLEWVSG
ISWNSGYIGYADSVKGRFTISRDNAENSLHLQMNSLRAEDTALYYCARGG
STLVRGVKGGYYGMDVWGQGTTVTVSSASTKGPSVFPLAPSSKSTSGGTA
ALGCLVKDYFPEPVTVSWNSGALTSGVHTFPAVLQSSGLYSLSSVVTVPS
SSLGTQTYICNVNHKPSNTKVDKKVEPKSCDKTH (SEQ ID NO: 620
)

### 69305

Fab Light Chain

DIQMTQSPSSLSASVGDRVTITCRASQSIDRYLNWYRQKPGKAPKLLIYT
TSSLQSGVPSRFSGSGSGTDFTLTLSSLQPEDFATYYCQQSYSPPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC(SEQ ID NO: 621)

Fab Heavy Chain

QVQLVESGGGVVQPGRSLRLSCAASGFTFSSYGMHWVRQAPGKGLEWVAV
IWYDGSNKYYADSVKGRFTISRDISKNTLYLQMNSLRAEDTAVYYCAGQL
DLFFDYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKKVEPKSCDKTH (SEQ ID NO: 622)

### 69307

Fab Light Chain

DIQMTQSPSSVSASVGDRVTITCRASQGISSWLAWYQQKPGKAPKLLIYA
ASSLQSGVPSRFSGSGSGTDFTLTISSLQPEDFATYYCQKADSLPYAFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC(SEQ ID NO: 623)

Fab Heavy Chain

EVQLVESGGGLVQPGGSLRLSCTASGFTFSNYWMTWVRQAPGKGLEWVAN
IKEDGSEKEYVDSVKGRFTISRDNAKNSLYLQMNSLRGEDTAVYYCARDG
EQLVDYYYYYVMDVWGQGTTVTVSSASTKGPSVFPLAPSSKSTSGGTAAL
GCLVKDYFPEPVTVSWNSGALTSGVHTFPAVLQSSGLYSLSSVVTVPSSS
LGTQTYICNVNHKPSNTKVDKKVEPKSCDKTH (SEQ ID NO: 624)

### 12795B

Fab Light Chain

DIQMTQSPSSLSASVGDRVTITCRASQGIRDHFGWYQQKPGKAPKRLIYA
ASSLHSGVPSRFSGSGSGTEFTLTISSLQPEDFATYYCLQYDTYPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC(SEQ ID NO: 625)

Fab Heavy Chain

EVQLVESGGGLVQPGGSLRLSCATSGFTFTSYDMKWVRQAPGLGLEWVSA
ISGSGGNTYYADSVKGRFTISRDNSRNTLYLQMNSLRAEDTAVYYCTRSH
DFGAFDYFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTH (SEQ ID NO: 626)

### 12798B (regn17078)

Fab Light Chain

EIVMTQSPATLSVSPGERATLSCRASQTVSSNLAWYQQKPGQAPRLLIYG
SSSRATGIPARFSGSGSGTEFTLTISSLQSEDFAVYYCQQYNNWPPYTFG
QGTKLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC(SEQ ID NO: 627)

Fab Heavy Chain

EVQLVESGGDLVQPGRSLRLSCAASGFTFDDYAMHWVRQAPGKGLEWVSG
ISWNSATRVYADSVKGRFTISRDNAKNFLYLQMNSLRSEDTALYHCAKDM
DISLGYYGLDVWGQGTTVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTH (SEQ ID NO: 628); or

EVQLVESGGDLVQPGRSLRLSCAASGFTFDDYAMHWVRQAPGKGLEWVSG
ISWNSATRVYADSVKGRFTISRDNAKNFLYLQMNSLRSEDTALYHCAKDM
DISLGYYGLDVWGQGTTVTVSSASTKGPSVFPLAPCSRSTSESTAALGCL
VKDYFPEPVTVSWNSGALTSGVHTFPAVLQSSGLYSLSSVVTVPSSSLGT
KTYTCNVDHKPSNTKVDKRVESKYGPPLLQGSG (SEQ ID NO: 667)

### 12799B (regn17079)

Fab Light Chain

DIQMTQSPSSVSASVGDRVTITCRASQGIASWLAWYQQKPGKAPELLIYA
ASSLQGGVPSRFSGSGSGTDFTLTISSLQPEDFAIYYCQQANYFPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC(SEQ ID NO: 629)

Fab Heavy Chain

QITLKESGPTLVKPTQTLTLTCTFSGFSLSTSGVGVVWIRQPPGKALEWL
ALIYWNDHKRYSPSLGSRLTITKDTSKNQVVLTMTNMDPVDTATYYCAHY
SGSYSYYYYGLDVWGQGTTVTVSSASTKGPSVFPLAPSSKSTSGGTAALG
CLVKDYFPEPVTVSWNSGALTSGVHTFPAVLQSSGLYSLSSVVTVPSSSL
GTQTYICNVNHKPSNTKVDKKVEPKSCDKTH (SEQ ID NO: 630);
or

QITLKESGPTLVKPTQTLTLTCTFSGFSLSTSGVGVVWIRQPPGKALEWL
ALIYWNDHKRYSPSLGSRLTITKDTSKNQVVLTMTNMDPVDTATYYCAHY
SGSYSYYYYGLDVWGQGTTVTVSSASTKGPSVFPLAPCSRSTSESTAALG
CLVKDYFPEPVTVSWNSGALTSGVHTFPAVLQSSGLYSLSSVVTVPSSSL
GTKTYTCNVDHKPSNTKVDKRVESKYGPPLLQGSG(SEQ ID NO: 668
)

### 12801B

Fab Light Chain

DIQMTQSPSSLSASVGDRVTITCRASQGIRTDLGWYQQKPGKAPKRLIYA
ASSLQSGVPSRFSGSGSGTEFTLTISSLRPEDFATFYCLQYNSYPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC(SEQ ID NO: 631)

Fab Heavy Chain

EVQLLESGGALVQPGGSLRLSCAASGFTFTSYAMHWVRQAPGKGLEWVSS
IRGSGGGTYSADSVKGRFTISRDNSRDTLYLOMNSVRAEDTAVYYCARSH
DYGAFDFFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTH (SEQ ID NO: 632)

### 12802B

Fab Light Chain

EIVMTQSPATLSVSPGERATLSCRASQSVSINLAWYQQKPGQAPRLLIFV
ASTRATGIPARFSGSGSGTEFTLTISSLQSEDFATYYCQQYDIWPYTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC(SEQ ID NO: 633)

Fab Heavy Chain

QVQLVESGGGLVKPGGSLRLSCAASGFTFSDYFMSWIRQAPGKGLEWVSY
ISSTGSTINYADSVKGRFTISRDNVKNSLYLQMTSLRVEDTAVYYCTRDN
WNYEYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTH (SEQ ID NO: 634)

### 12808B

Fab Light Chain

DIQMTQSPSSLSASVGDRVTINCRASQGIRNDLGWYQQKPGKAPKRLIYA
ASSLQSGVPLRFSGSGSGTEFTLTINNLQPEDFATYYCLSHNSYPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC(SEQ ID NO: 635)

Fab Heavy Chain

QLQLQESGPGLVKPSETLSLTCTVSGESISSNTYYWGWIRQPPGKGLEWI
GSIDYSGTTNYNPSLKSRVTISVDTSRNHFSLRLRSVTAADTAVYYCARE
WGNYGYYYGMDVWGQGTTVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTH (SEQ ID NO: 636)

### 12812B

Fab Light Chain

DIQMTQSPPSVSASVGDRVTITCRASQGISSWLAWYQQKPGKAPKLLIYA
ASSLQSGVPSRFSGSGSGTDFTLTISSLQPEDFATYYCQQANSFPRTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC(SEQ ID NO: 637)

Fab Heavy Chain

QVQLVQSGAEVKKPGSSVRVSCKASRGTFSSYAISWVRQAPGQGLEWMGG
IIPIFGTANYAQKFLARVTITADESTSTAYMELSSLRSEDTAVYYCAREK
GWNYFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTH (SEQ ID NO: 638)

### 12816B

Fab Light Chain

DIVMTQSPLSLPVTPGEPASISCRSSQSLLHGNGYNYLTWYLQKPGQSPQ
LLIYLGSNRASGVPDRFSGSGSGTDFTLKISRVEAEDVGVYYCMQALQTP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC (SEQ ID NO: 639)

Fab Heavy Chain

QVQLVESGGGLVKPGGSLRLSCAASGFTFSDYYMNWIRQAPGKGLEWVSY
ISSSGTTIYYADSVKGRFTISRDNAKKSLYLEMNSLRAEDTAVYYCAREG
YGNDYYYYGIDVWGQGTTVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTH (SEQ ID NO: 640)

### 12833B

Fab Light Chain

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPITFG
QGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC(SEQ ID NO: 641)

Fab Heavy Chain

QVQLVESGGGVVQPGRSLRLSCAASGFTFSSFGMHWVRQAPGKGLEWVIF
ISYDGSDKYYADSVKGRFAISRDSSKNTLYLQMNSLRAEDTAVYYCAKEN
GILTDSYGMDVWGQGTTVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTH (SEQ ID NO: 642)

### 12834B

Fab Light Chain

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPITFG
QGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC(SEQ ID NO: 643)

Fab Heavy Chain

QVQLVQSGAEVKKPGASVKVSCKASGYTFTSYGISWVRQAPGQGLEWMGW
ISVYHGNTNYAQKFQGRVTMTTDTSTSTAYMELRSLRSDDTAVYYCAREG
YYDFWSGYYPFDYWGQGTLVTVSSASTKGPSVFPLAPSSKSTSGGTAALG
CLVKDYFPEPVTVSWNSGALTSGVHTFPAVLQSSGLYSLSSVVTVPSSSL
GTQTYICNVNHKPSNTKVDKKVEPKSCDKTH (SEQ ID NO: 644)

### 12835B

Fab Light Chain

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPITFG
QGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC(SEQ ID NO: 645)

Fab Heavy Chain

EVQLVESGGGLIQPGGSLRLSCEASGFTFRNYEMNWVRQAPGKGLEWVSY
ISSSGNMKDYAESVKGRFTISRDNVKNSLQLQMNSLRVEDTAVYYCARDE
FPYGMDVWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTH (SEQ ID NO: 646)

### 12847B (regn17083)

Fab Light Chain

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPITFG
QGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC(SEQ ID NO: 647)

Fab Heavy Chain

EVQLVESGGGLVQPGRSLRLSCAASGFTFDDYAMNWVRQAPGKGLEWVSG
ISWSSGSMDYADSVKGRFTISRDNAKNSLYLQMNSLRTEDTALYYCAKAR
EVGDYYGMDVWGQGTTVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTH (SEQ ID NO: 648); or

EVQLVESGGGLVQPGRSLRLSCAASGFTFDDYAMNWVRQAPGKGLEWVSG
ISWSSGSMDYADSVKGRFTISRDNAKNSLYLQMNSLRTEDTALYYCAKAR
EVGDYYGMDVWGQGTTVTVSSASTKGPSVFPLAPCSRSTSESTAALGCLV
KDYFPEPVTVSWNSGALTSGVHTFPAVLQSSGLYSLSSVVTVPSSSLGTK
TYTCNVDHKPSNTKVDKRVESKYGPPLLQGSG(SEQ ID NO: 669)

### 12848B

Fab Light Chain

EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPWTFG
QGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC(SEQ ID NO: 649)

Fab Heavy Chain

EVQLVESGGGLVQPGRSLTLSCAASGFTFDNFGMHWVRQGPGKGLEWVSG
LTWNSGVIGYADSVKGRFTISRDNAKNSLYLQMNSLRPEDTALYYCAKDI
RNYGPFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTH (SEQ ID NO: 650)

### 12843B (regn17081)

Fab Light Chain

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPITFG
QGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC(SEQ ID NO: 651)

Fab Heavy Chain

EVQLVESGGGLVQPGGSLRLSCAASGFTFNIFEMNWVRQAPGKGLEWISY
ISSRGTTTYYADSVRGRFTISRDNAKNSLYLQMNSLRAEDTAVYYCARDY
EATIPFDFWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTH (SEQ ID NO: 652); or

EVQLVESGGGLVQPGGSLRLSCAASGFTFNIFEMNWVRQAPGKGLEWISY
ISSRGTTTYYADSVRGRFTISRDNAKNSLYLQMNSLRAEDTAVYYCARDY
EATIPFDFWGQGTLVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPLLQGSG(SEQ ID NO: 670)

### 12844B

Fab Light Chain

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPITFG
QGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC(SEQ ID NO: 653)

Fab Heavy Chain

EVQLVESGGSVVRPGGSLRLSCEASGFTFDDYGMSWVRQDPGKGLEWVSG
INWNGDRTNYADSVKGRFIISRDNAKNSVYLQMNSLRAEDSALYHCARDQ
GLGVAATLDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTH (SEQ ID NO: 654)

### 12845B (regn17082)

Fab Light Chain

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPITFG
QGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC(SEQ ID NO: 655)

Fab Heavy Chain

EVQLVESGGGLVQPGGSLRLSCAASGFTVSNYEMNWVRQAPGKGLEWVSY
ISSSTSNIYYADSVKGRFTISRDNAENSLYLQMNSLRVEDTAVYYCVRDG
IVVVPVGRGYYYYGLDVWGQGTTVTVSSASTKGPSVFPLAPSSKSTSGGT
AALGCLVKDYFPEPVTVSWNSGALTSGVHTFPAVLQSSGLYSLSSVVTVP
SSSLGTQTYICNVNHKPSNTKVDKKVEPKSCDKTH (SEQ ID NO: 65
6); or

EVQLVESGGGLVQPGGSLRLSCAASGFTVSNYEMNWVRQAPGKGLEWVSY
ISSSTSNIYYADSVKGRFTISRDNAENSLYLQMNSLRVEDTAVYYCVRDG
IVVVPVGRGYYYYGLDVWGQGTTVTVSSASTKGPSVFPLAPCSRSTSEST
AALGCLVKDYFPEPVTVSWNSGALTSGVHTFPAVLQSSGLYSLSSVVTVP
SSSLGTKTYTCNVDHKPSNTKVDKRVESKYGPPLLQGSG(SEQ ID NO:
671)

### 12839B (regn17080)

Fab Light Chain

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPITFG
QGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC(SEQ ID NO: 657)

Fab Heavy Chain

QVQLVESGGGVVQPGRSLRLSCAASGFPFSNYVMYWVRQAPGKGLEWVAL
IFFDGKKNYHADSVKGRFTITRDNSKNMLYLQMNSLRPEDAAVYYCAKIH
CPNGVCYKGYYGMDVWGQGTTVTVSSASTKGPSVFPLAPSSKSTSGGTAA
LGCLVKDYFPEPVTVSWNSGALTSGVHTFPAVLQSSGLYSLSSVVTVPSS
SLGTQTYICNVNHKPSNTKVDKKVEPKSCDKTH (SEQ ID NO: 658)
; or

QVQLVESGGGVVQPGRSLRLSCAASGFPFSNYVMYWVRQAPGKGLEWVAL
IFFDGKKNYHADSVKGRFTITRDNSKNMLYLQMNSLRPEDAAVYYCAKIH
CPNGVCYKGYYGMDVWGQGTTVTVSSASTKGPSVFPLAPCSRSTSESTAA
LGCLVKDYFPEPVTVSWNSGALTSGVHTFPAVLQSSGLYSLSSVVTVPSS
SLGTKTYTCNVDHKPSNTKVDKRVESKYGPPLLQGSG(SEQ ID NO: 6
72)

### Hih12841b

Fab Light Chain

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPITFG
QGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC(SEQ ID NO: 659)

Fab Heavy Chain

EVQLVESGGGLVQPGGSLRLSCAASGFTFSNYWMNWVRQAPGKGLEWVAN
IKEDGGKKLYVDSVKGRFTISRDNAKNSLFLQMNSLRAEDTAVYYCARED
TTLVVDYYYYGMDVWGQGTTVTVSSASTKGPSVFPLAPSSKSTSGGTAAL
GCLVKDYFPEPVTVSWNSGALTSGVHTFPAVLQSSGLYSLSSVVTVPSSS
LGTQTYICNVNHKPSNTKVDKKVEPKSCDKTH (SEQ ID NO: 660)

### 12850B

Fab Light Chain

EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPWTFG
QGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC(SEQ ID NO: 661)

Fab Heavy Chain

QVQLVQSGAEVKKPGSSVKVSCKASGGTFNTYAITWVRQAPGQGLEWMGG
IIPISGIAEYAQKFQGRVTITTDDSSTTAYMELNSLRSEDTAVYYCASWN
YALYYFYGMDVWGRGTTVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTH (SEQ ID NO: 662)

### 69261

Fab Light Chain

DIVMTQSPLSLPVTPGEPASISCRSSQSLLHSNGYNYLDWYLQKPGQSPQ
FLIYLGSNRASGVPDRFSGSGSGTDFTLKINRVEAEDVGVYYCMQALQTP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC (SEQ ID NO: 663)

Fab Heavy Chain

QVQLVESGGGLVKPGGSLRLSCAASGFTFSVYYMNWIRQAPGKGLEWVSY
ISSSGSTIYYADSVKGRFTISRDNAKNSLYLQMNSLRAEDTAVYYCGREG
YSGTYSYYGMDVWGQGTTVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTH (SEQ ID NO: 664)

### 69263

Fab Light Chain

DIQMTQSPSSLSASVGDRVTITCRASQDISHYSAWYQQKPGKLPNLLIYA
ASTLQSGVPSRFSGSGSGTDFSLTTSSLQPEDVATYYCQKYNSVPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC(SEQ ID NO: 665)

Fab Heavy Chain

EVQLVESGGGLVQPGRSLRLSCAVSGFTFDDYAMHWVRQAPGKGLEWVSG
ISWNSGTRGYADSVKGRFTISRDNAKNSLYLQMNSLRGEDTALYYCVKDI
TISPNYYGMDVWGQGTTVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTH (SEQ ID NO: 666)

### Anti-TfR scFVGAA Sequences

12795B

DIQMTQSPSSLSASVGDRVTITCRASQGIRDHFGWYQQKPGKAPKRLIYA
ASSLHSGVPSRFSGSGSGTEFTLTISSLQPEDFATYYCLQYDTYPLTFGG
GTKVEIKGGGGSGGGGSGGGGSEVQLVESGGGLVQPGGSLRLSCATSGFT
FTSYDMKWVRQAPGLGLEWVSAISGSGGNTYYADSVKGRFTISRDNSRNT
LYLQMNSLRAEDTAVYYCTRSHDFGAFDYFDYWGQGTLVTVSSGGGGSGG
GGSAHPGRPRAVPTQCDVPPNSRFDCAPDKAITQEQCEARGCCYIPAKQG
LQGAQMGQPWCFFPPSYPSYKLENLSSSEMGYTATLTRTTPTFFPKDILT
LRLDVMMETENRLHFTIKDPANRRYEVPLETPHVHSRAPSPLYSVEFSEE
PFGVIVRRQLDGRVLLNTTVAPLFFADQFLQLSTSLPSQYITGLAEHLSP
LMLSTSWTRITLWNRDLAPTPGANLYGSHPFYLALEDGGSAHGVFLLNSN
AMDVVLQPSPALSWRSTGGILDVYIFLGPEPKSVVQQYLDVVGYPFMPPY
WGLGFHLCRWGYSSTAITRQVVENMTRAHFPLDVQWNDLDYMDSRRDFTF
NKDGFRDFPAMVQELHQGGRRYMMIVDPAISSSGPAGSYRPYDEGLRRGV
FITNETGQPLIGKVWPGSTAFPDFTNPTALAWWEDMVAEFHDQVPFDGMW
IDMNEPSNFIRGSEDGCPNNELENPPYVPGVVGGTLQAATICASSHQFLS
THYNLHNLYGLTEAIASHRALVKARGTRPFVISRSTFAGHGRYAGHWTGD
VWSSWEQLASSVPEILQFNLLGVPLVGADVCGFLGNTSEELCVRWTQLGA
FYPFMRNHNSLLSLPQEPYSFSEPAQQAMRKALTLRYALLPHLYTLFHQA
HVAGETVARPLFLEFPKDSSTWTVDHQLLWGEALLITPVLQAGKAEVTGY
FPLGTWYDLQTVPVEALGSLPPPPAAPREPAIHSEGQWVTLPAPLDTINV
HLRAGYIIPLQGPGLTTTESRQQPMALAVALTKGGEARGELFWDDGESLE
VLERGAYTQVIFLARNNTIVNELVRVTSEGAGLQLQKVTVLGVATAPQQV
LSNGVPVSNFTYSPDTKVLDICVSLLMGEQFLVSWC(SEQ ID NO: 67
5)

12798B (REGN16818)

EIVMTQSPATLSVSPGERATLSCRASQTVSSNLAWYQQKPGQAPRLLIYG
SSSRATGIPARFSGSGSGTEFTLTISSLQSEDFAVYYCQQYNNWPPYTFG
QGTKLEIKGGGGSGGGGSGGGGSEVQLVESGGDLVQPGRSLRLSCAASGF
TFDDYAMHWVRQAPGKGLEWVSGISWNSATRVYADSVKGRFTISRDNAKN
FLYLQMNSLRSEDTALYHCAKDMDISLGYYGLDVWGQGTTVTVSSGGGGS
GGGGSAHPGRPRAVPTQCDVPPNSRFDCAPDKAITQEQCEARGCCYIPAK
QGLQGAQMGQPWCFFPPSYPSYKLENLSSSEMGYTATLTRTTPTFFPKDI
LTLRLDVMMETENRLHFTIKDPANRRYEVPLETPHVHSRAPSPLYSVEFS
EEPFGVIVRRQLDGRVLLNTTVAPLFFADQFLQLSTSLPSQYITGLAEHL
SPLMLSTSWTRITLWNRDLAPTPGANLYGSHPFYLALEDGGSAHGVFLLN
SNAMDVVLQPSPALSWRSTGGILDVYIFLGPEPKSVVQQYLDVVGYPFMP
PYWGLGFHLCRWGYSSTAITRQVVENMTRAHFPLDVQWNDLDYMDSRRDF
TFNKDGFRDFPAMVQELHQGGRRYMMIVDPAISSSGPAGSYRPYDEGLRR
GVFITNETGQPLIGKVWPGSTAFPDFTNPTALAWWEDMVAEFHDQVPFDG
MWIDMNEPSNFIRGSEDGCPNNELENPPYVPGVVGGTLQAATICASSHQF
LSTHYNLHNLYGLTEAIASHRALVKARGTRPFVISRSTFAGHGRYAGHWT
GDVWSSWEQLASSVPEILQFNLLGVPLVGADVCGFLGNTSEELCVRWTQL
GAFYPFMRNHNSLLSLPQEPYSFSEPAQQAMRKALTLRYALLPHLYTLFH
QAHVAGETVARPLFLEFPKDSSTWTVDHQLLWGEALLITPVLQAGKAEVT
GYFPLGTWYDLQTVPVEALGSLPPPPAAPREPAIHSEGQWVTLPAPLDTI
NVHLRAGYIIPLQGPGLTTTESRQQPMALAVALTKGGEARGELFWDDGES
LEVLERGAYTQVIFLARNNTIVNELVRVTSEGAGLQLQKVTVLGVATAPQ
QVLSNGVPVSNFTYSPDTKVLDICVSLLMGEQFLVSWC(SEQ ID NO:
676)

12799B (REGN16819)

DIQMTQSPSSVSASVGDRVTITCRASQGIASWLAWYQQKPGKAPELLIYA
ASSLQGGVPSRFSGSGSGTDFTLTISSLQPEDFAIYYCQQANYFPWTFGQ
GTKVEIKGGGGSGGGGSGGGGSQITLKESGPTLVKPTQTLTLTCTFSGFS
LSTSGVGVVWIRQPPGKALEWLALIYWNDHKRYSPSLGSRLTITKDTSKN
QVVLTMTNMDPVDTATYYCAHYSGSYSYYYYGLDVWGQGTTVTVSSGGGG
SGGGGSAHPGRPRAVPTQCDVPPNSRFDCAPDKAITQEQCEARGCCYIPA
KQGLQGAQMGQPWCFFPPSYPSYKLENLSSSEMGYTATLTRTTPTFFPKD
ILTLRLDVMMETENRLHFTIKDPANRRYEVPLETPHVHSRAPSPLYSVEF
SEEPFGVIVRRQLDGRVLLNTTVAPLFFADQFLQLSTSLPSQYITGLAEH
LSPLMLSTSWTRITLWNRDLAPTPGANLYGSHPFYLALEDGGSAHGVFLL
NSNAMDVVLQPSPALSWRSTGGILDVYIFLGPEPKSVVQQYLDVVGYPFM
PPYWGLGFHLCRWGYSSTAITRQVVENMTRAHFPLDVQWNDLDYMDSRRD
FTFNKDGFRDFPAMVQELHQGGRRYMMIVDPAISSSGPAGSYRPYDEGLR
RGVFITNETGQPLIGKVWPGSTAFPDFTNPTALAWWEDMVAEFHDQVPFD
GMWIDMNEPSNFIRGSEDGCPNNELENPPYVPGVVGGTLQAATICASSHQ
FLSTHYNLHNLYGLTEAIASHRALVKARGTRPFVISRSTFAGHGRYAGHW
TGDVWSSWEQLASSVPEILQFNLLGVPLVGADVCGFLGNTSEELCVRWTQ
LGAFYPFMRNHNSLLSLPQEPYSFSEPAQQAMRKALTLRYALLPHLYTLF
HQAHVAGETVARPLFLEFPKDSSTWTVDHQLLWGEALLITPVLQAGKAEV
TGYFPLGTWYDLQTVPVEALGSLPPPPAAPREPAIHSEGQWVTLPAPLDT
INVHLRAGYIIPLQGPGLTTTESRQQPMALAVALTKGGEARGELFWDDGE
SLEVLERGAYTQVIFLARNNTIVNELVRVTSEGAGLQLQKVTVLGVATAP
QQVLSNGVPVSNFTYSPDTKVLDICVSLLMGEQFLVSWC(SEQ ID NO:
570)

12801B

DIQMTQSPSSLSASVGDRVTITCRASQGIRTDLGWYQQKPGKAPKRLIYA
ASSLQSGVPSRFSGSGSGTEFTLTISSLRPEDFATFYCLQYNSYPLTFGG
GTKVEIKGGGGSGGGGSGGGGSEVQLLESGGALVQPGGSLRLSCAASGFT
FTSYAMHWVRQAPGKGLEWVSSIRGSGGGTYSADSVKGRFTISRDNSRDT
LYLQMNSVRAEDTAVYYCARSHDYGAFDFFDYWGQGTLVTVSSGGGGSGG
GGSAHPGRPRAVPTQCDVPPNSRFDCAPDKAITQEQCEARGCCYIPAKQG
LQGAQMGQPWCFFPPSYPSYKLENLSSSEMGYTATLTRTTPTFFPKDILT
LRLDVMMETENRLHFTIKDPANRRYEVPLETPHVHSRAPSPLYSVEFSEE
PFGVIVRRQLDGRVLLNTTVAPLFFADQFLQLSTSLPSQYITGLAEHLSP
LMLSTSWTRITLWNRDLAPTPGANLYGSHPFYLALEDGGSAHGVFLLNSN
AMDVVLQPSPALSWRSTGGILDVYIFLGPEPKSVVQQYLDVVGYPFMPPY
WGLGFHLCRWGYSSTAITRQVVENMTRAHFPLDVQWNDLDYMDSRRDFTF
NKDGFRDFPAMVQELHQGGRRYMMIVDPAISSSGPAGSYRPYDEGLRRGV
FITNETGQPLIGKVWPGSTAFPDFTNPTALAWWEDMVAEFHDQVPFDGMW
IDMNEPSNFIRGSEDGCPNNELENPPYVPGVVGGTLQAATICASSHQFLS
THYNLHNLYGLTEAIASHRALVKARGTRPFVISRSTFAGHGRYAGHWTGD
VWSSWEQLASSVPEILQFNLLGVPLVGADVCGFLGNTSEELCVRWTQLGA
FYPFMRNHNSLLSLPQEPYSFSEPAQQAMRKALTLRYALLPHLYTLFHQA
HVAGETVARPLFLEFPKDSSTWTVDHQLLWGEALLITPVLQAGKAEVTGY
FPLGTWYDLQTVPVEALGSLPPPPAAPREPAIHSEGQWVTLPAPLDTINV
HLRAGYIIPLQGPGLTTTESRQQPMALAVALTKGGEARGELFWDDGESLE
VLERGAYTQVIFLARNNTIVNELVRVTSEGAGLQLQKVTVLGVATAPQQV
LSNGVPVSNFTYSPDTKVLDICVSLLMGEQFLVSWC(SEQ ID NO: 67
7)

12802B (REGN16820)

EIVMTQSPATLSVSPGERATLSCRASQSVSINLAWYQQKPGQAPRLLIFV
ASTRATGIPARFSGSGSGTEFTLTISSLQSEDFATYYCQQYDIWPYTFGQ
GTKLEIKGGGGSGGGGSGGGGSQVQLVESGGGLVKPGGSLRLSCAASGFT
FSDYFMSWIRQAPGKGLEWVSYISSTGSTINYADSVKGRFTISRDNVKNS
LYLQMTSLRVEDTAVYYCTRDNWNYEYWGQGTLVTVSSGGGGSGGGGSAH
PGRPRAVPTQCDVPPNSRFDCAPDKAITQEQCEARGCCYIPAKQGLQGAQ
MGQPWCFFPPSYPSYKLENLSSSEMGYTATLTRTTPTFFPKDILTLRLDV
MMETENRLHFTIKDPANRRYEVPLETPHVHSRAPSPLYSVEFSEEPFGVI
VRRQLDGRVLLNTTVAPLFFADQFLQLSTSLPSQYITGLAEHLSPLMLST
SWTRITLWNRDLAPTPGANLYGSHPFYLALEDGGSAHGVFLLNSNAMDVV
LQPSPALSWRSTGGILDVYIFLGPEPKSVVQQYLDVVGYPFMPPYWGLGF
HLCRWGYSSTAITRQVVENMTRAHFPLDVQWNDLDYMDSRRDFTFNKDGF
RDFPAMVQELHQGGRRYMMIVDPAISSSGPAGSYRPYDEGLRRGVFITNE
TGQPLIGKVWPGSTAFPDFTNPTALAWWEDMVAEFHDQVPFDGMWIDMNE
PSNFIRGSEDGCPNNELENPPYVPGVVGGTLQAATICASSHQFLSTHYNL
HNLYGLTEAIASHRALVKARGTRPFVISRSTFAGHGRYAGHWTGDVWSSW
EQLASSVPEILQFNLLGVPLVGADVCGFLGNTSEELCVRWTQLGAFYPFM
RNHNSLLSLPQEPYSFSEPAQQAMRKALTLRYALLPHLYTLFHQAHVAGE
TVARPLFLEFPKDSSTWTVDHQLLWGEALLITPVLQAGKAEVTGYFPLGT
WYDLQTVPVEALGSLPPPPAAPREPAIHSEGQWVTLPAPLDTINVHLRAG
YIIPLQGPGLTTTESRQQPMALAVALTKGGEARGELFWDDGESLEVLERG
AYTQVIFLARNNTIVNELVRVTSEGAGLQLQKVTVLGVATAPQQVLSNGV
PVSNFTYSPDTKVLDICVSLLMGEQFLVSWC(SEQ ID NO: 678)

12808B

DIQMTQSPSSLSASVGDRVTINCRASQGIRNDLGWYQQKPGKAPKRLIYA
ASSLQSGVPLRFSGSGSGTEFTLTINNLQPEDFATYYCLSHNSYPWTFGQ
GTKVEIKGGGGSGGGGSGGGGSQLQLQESGPGLVKPSETLSLTCTVSGES
ISSNTYYWGWIRQPPGKGLEWIGSIDYSGTTNYNPSLKSRVTISVDTSRN
HFSLRLRSVTAADTAVYYCAREWGNYGYYYGMDVWGQGTTVTVSSGGGGS
GGGGSAHPGRPRAVPTQCDVPPNSRFDCAPDKAITQEQCEARGCCYIPAK
QGLQGAQMGQPWCFFPPSYPSYKLENLSSSEMGYTATLTRTTPTFFPKDI
LTLRLDVMMETENRLHFTIKDPANRRYEVPLETPHVHSRAPSPLYSVEFS
EEPFGVIVRRQLDGRVLLNTTVAPLFFADQFLQLSTSLPSQYITGLAEHL
SPLMLSTSWTRITLWNRDLAPTPGANLYGSHPFYLALEDGGSAHGVFLLN
SNAMDVVLQPSPALSWRSTGGILDVYIFLGPEPKSVVQQYLDVVGYPFMP
PYWGLGFHLCRWGYSSTAITRQVVENMTRAHFPLDVQWNDLDYMDSRRDF
TFNKDGFRDFPAMVQELHQGGRRYMMIVDPAISSSGPAGSYRPYDEGLRR
GVFITNETGQPLIGKVWPGSTAFPDFTNPTALAWWEDMVAEFHDQVPFDG
MWIDMNEPSNFIRGSEDGCPNNELENPPYVPGVVGGTLQAATICASSHQF
LSTHYNLHNLYGLTEAIASHRALVKARGTRPFVISRSTFAGHGRYAGHWT
GDVWSSWEQLASSVPEILQFNLLGVPLVGADVCGFLGNTSEELCVRWTQL
GAFYPFMRNHNSLLSLPQEPYSFSEPAQQAMRKALTLRYALLPHLYTLFH
QAHVAGETVARPLFLEFPKDSSTWTVDHQLLWGEALLITPVLQAGKAEVT
GYFPLGTWYDLQTVPVEALGSLPPPPAAPREPAIHSEGQWVTLPAPLDTI
NVHLRAGYIIPLQGPGLTTTESRQQPMALAVALTKGGEARGELFWDDGES
LEVLERGAYTQVIFLARNNTIVNELVRVTSEGAGLQLQKVTVLGVATAPQ
QVLSNGVPVSNFTYSPDTKVLDICVSLLMGEQFLVSWC(SEQ ID NO:
679)

12812B (REGN16821)

DIQMTQSPPSVSASVGDRVTITCRASQGISSWLAWYQQKPGKAPKLLIYA
ASSLQSGVPSRFSGSGSGTDFTLTISSLQPEDFATYYCQQANSFPRTFGQ
GTKVEIKGGGGSGGGGSGGGGSQVQLVQSGAEVKKPGSSVRVSCKASRGT
FSSYAISWVRQAPGQGLEWMGGIIPIFGTANYAQKFLARVTITADESTST
AYMELSSLRSEDTAVYYCAREKGWNYFDYWGQGTLVTVSSGGGGSGGGGS
AHPGRPRAVPTQCDVPPNSRFDCAPDKAITQEQCEARGCCYIPAKQGLQG
AQMGQPWCFFPPSYPSYKLENLSSSEMGYTATLTRTTPTFFPKDILTLRL
DVMMETENRLHFTIKDPANRRYEVPLETPHVHSRAPSPLYSVEFSEEPFG
VIVRRQLDGRVLLNTTVAPLFFADQFLQLSTSLPSQYITGLAEHLSPLML
STSWTRITLWNRDLAPTPGANLYGSHPFYLALEDGGSAHGVFLLNSNAMD
VVLQPSPALSWRSTGGILDVYIFLGPEPKSVVQQYLDVVGYPFMPPYWGL
GFHLCRWGYSSTAITRQVVENMTRAHFPLDVQWNDLDYMDSRRDFTFNKD
GFRDFPAMVQELHQGGRRYMMIVDPAISSSGPAGSYRPYDEGLRRGVFIT
NETGQPLIGKVWPGSTAFPDFTNPTALAWWEDMVAEFHDQVPFDGMWIDM
NEPSNFIRGSEDGCPNNELENPPYVPGVVGGTLQAATICASSHQFLSTHY
NLHNLYGLTEAIASHRALVKARGTRPFVISRSTFAGHGRYAGHWTGDVWS
SWEQLASSVPEILQFNLLGVPLVGADVCGFLGNTSEELCVRWTQLGAFYP
FMRNHNSLLSLPQEPYSFSEPAQQAMRKALTLRYALLPHLYTLFHQAHVA
GETVARPLFLEFPKDSSTWTVDHQLLWGEALLITPVLQAGKAEVTGYFPL
GTWYDLQTVPVEALGSLPPPPAAPREPAIHSEGQWVTLPAPLDTINVHLR
AGYIIPLQGPGLTTTESRQQPMALAVALTKGGEARGELFWDDGESLEVLE
RGAYTQVIFLARNNTIVNELVRVTSEGAGLQLQKVTVLGVATAPQQVLSN
GVPVSNFTYSPDTKVLDICVSLLMGEQFLVSWC(SEQ ID NO: 680)

12816B

DIVMTQSPLSLPVTPGEPASISCRSSQSLLHGNGYNYLTWYLQKPGQSPQ
LLIYLGSNRASGVPDRFSGSGSGTDFTLKISRVEAEDVGVYYCMQALQTP
YTFGQGTKLEIKGGGGSGGGGSGGGGSQVQLVESGGGLVKPGGSLRLSCA
ASGFTFSDYYMNWIRQAPGKGLEWVSYISSSGTTIYYADSVKGRFTISRD
NAKKSLYLEMNSLRAEDTAVYYCAREGYGNDYYYYGIDVWGQGTTVTVSS
GGGGSGGGGSAHPGRPRAVPTQCDVPPNSRFDCAPDKAITQEQCEARGCC
YIPAKQGLQGAQMGQPWCFFPPSYPSYKLENLSSSEMGYTATLTRTTPTF
FPKDILTLRLDVMMETENRLHFTIKDPANRRYEVPLETPHVHSRAPSPLY
SVEFSEEPFGVIVRRQLDGRVLLNTTVAPLFFADQFLQLSTSLPSQYITG
LAEHLSPLMLSTSWTRITLWNRDLAPTPGANLYGSHPFYLALEDGGSAHG
VFLLNSNAMDVVLQPSPALSWRSTGGILDVYIFLGPEPKSVVQQYLDVVG
YPFMPPYWGLGFHLCRWGYSSTAITRQVVENMTRAHFPLDVQWNDLDYMD
SRRDFTFNKDGFRDFPAMVQELHQGGRRYMMIVDPAISSSGPAGSYRPYD
EGLRRGVFITNETGQPLIGKVWPGSTAFPDFTNPTALAWWEDMVAEFHDQ
VPFDGMWIDMNEPSNFIRGSEDGCPNNELENPPYVPGVVGGTLQAATICA
SSHQFLSTHYNLHNLYGLTEAIASHRALVKARGTRPFVISRSTFAGHGRY
AGHWTGDVWSSWEQLASSVPEILQFNLLGVPLVGADVCGFLGNTSEELCV
RWTQLGAFYPFMRNHNSLLSLPQEPYSFSEPAQQAMRKALTLRYALLPHL
YTLFHQAHVAGETVARPLFLEFPKDSSTWTVDHQLLWGEALLITPVLQAG
KAEVTGYFPLGTWYDLQTVPVEALGSLPPPPAAPREPAIHSEGQWVTLPA
PLDTINVHLRAGYIIPLQGPGLTTTESRQQPMALAVALTKGGEARGELFW
DDGESLEVLERGAYTQVIFLARNNTIVNELVRVTSEGAGLQLQKVTVLGV
ATAPQQVLSNGVPVSNFTYSPDTKVLDICVSLLMGEQFLVSWC(SEQ ID
NO: 681)

12833B

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPITFG
QGTRLEIKGGGGSGGGGSGGGGSQVQLVESGGGVVQPGRSLRLSCAASGF
TFSSFGMHWVRQAPGKGLEWVIFISYDGSDKYYADSVKGRFAISRDSSKN
TLYLQMNSLRAEDTAVYYCAKENGILTDSYGMDVWGQGTTVTVSSGGGGS
GGGGSAHPGRPRAVPTQCDVPPNSRFDCAPDKAITQEQCEARGCCYIPAK
QGLQGAQMGQPWCFFPPSYPSYKLENLSSSEMGYTATLTRTTPTFFPKDI
LTLRLDVMMETENRLHFTIKDPANRRYEVPLETPHVHSRAPSPLYSVEFS
EEPFGVIVRRQLDGRVLLNTTVAPLFFADQFLQLSTSLPSQYITGLAEHL
SPLMLSTSWTRITLWNRDLAPTPGANLYGSHPFYLALEDGGSAHGVFLLN
SNAMDVVLQPSPALSWRSTGGILDVYIFLGPEPKSVVQQYLDVVGYPFMP
PYWGLGFHLCRWGYSSTAITRQVVENMTRAHFPLDVQWNDLDYMDSRRDF
TFNKDGFRDFPAMVQELHQGGRRYMMIVDPAISSSGPAGSYRPYDEGLRR
GVFITNETGQPLIGKVWPGSTAFPDFTNPTALAWWEDMVAEFHDQVPFDG
MWIDMNEPSNFIRGSEDGCPNNELENPPYVPGVVGGTLQAATICASSHQF
LSTHYNLHNLYGLTEAIASHRALVKARGTRPFVISRSTFAGHGRYAGHWT
GDVWSSWEQLASSVPEILQFNLLGVPLVGADVCGFLGNTSEELCVRWTQL
GAFYPFMRNHNSLLSLPQEPYSFSEPAQQAMRKALTLRYALLPHLYTLFH
QAHVAGETVARPLFLEFPKDSSTWTVDHQLLWGEALLITPVLQAGKAEVT
GYFPLGTWYDLQTVPVEALGSLPPPPAAPREPAIHSEGQWVTLPAPLDTI
NVHLRAGYIIPLQGPGLTTTESRQQPMALAVALTKGGEARGELFWDDGES
LEVLERGAYTQVIFLARNNTIVNELVRVTSEGAGLQLQKVTVLGVATAPQ
QVLSNGVPVSNFTYSPDTKVLDICVSLLMGEQFLVSWC(SEQ ID NO:
682)

12834B

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPITFG
QGTRLEIKGGGGSGGGGSGGGGSQVQLVQSGAEVKKPGASVKVSCKASGY
TFTSYGISWVRQAPGQGLEWMGWISVYHGNTNYAQKFQGRVTMTTDTSTS
TAYMELRSLRSDDTAVYYCAREGYYDFWSGYYPFDYWGQGTLVTVSSGGG
GSGGGGSAHPGRPRAVPTQCDVPPNSRFDCAPDKAITQEQCEARGCCYIP
AKQGLQGAQMGQPWCFFPPSYPSYKLENLSSSEMGYTATLTRTTPTFFPK
DILTLRLDVMMETENRLHFTIKDPANRRYEVPLETPHVHSRAPSPLYSVE
FSEEPFGVIVRRQLDGRVLLNTTVAPLFFADQFLQLSTSLPSQYITGLAE
HLSPLMLSTSWTRITLWNRDLAPTPGANLYGSHPFYLALEDGGSAHGVFL
LNSNAMDVVLQPSPALSWRSTGGILDVYIFLGPEPKSVVQQYLDVVGYPF
MPPYWGLGFHLCRWGYSSTAITRQVVENMTRAHFPLDVQWNDLDYMDSRR
DFTFNKDGFRDFPAMVQELHQGGRRYMMIVDPAISSSGPAGSYRPYDEGL
RRGVFITNETGQPLIGKVWPGSTAFPDFTNPTALAWWEDMVAEFHDQVPF
DGMWIDMNEPSNFIRGSEDGCPNNELENPPYVPGVVGGTLQAATICASSH
QFLSTHYNLHNLYGLTEAIASHRALVKARGTRPFVISRSTFAGHGRYAGH
WTGDVWSSWEQLASSVPEILQFNLLGVPLVGADVCGFLGNTSEELCVRWT
QLGAFYPFMRNHNSLLSLPQEPYSFSEPAQQAMRKALTLRYALLPHLYTL
FHQAHVAGETVARPLFLEFPKDSSTWTVDHQLLWGEALLITPVLQAGKAE
VTGYFPLGTWYDLQTVPVEALGSLPPPPAAPREPAIHSEGQWVTLPAPLD
TINVHLRAGYIIPLQGPGLTTTESRQQPMALAVALTKGGEARGELFWDDG
ESLEVLERGAYTQVIFLARNNTIVNELVRVTSEGAGLQLQKVTVLGVATA
PQQVLSNGVPVSNFTYSPDTKVLDICVSLLMGEQFLVSWC(SEQ ID NO
: 683)

12835B

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPITFG
QGTRLEIKGGGGSGGGGSGGGGSEVQLVESGGGLIQPGGSLRLSCEASGF
TFRNYEMNWVRQAPGKGLEWVSYISSSGNMKDYAESVKGRFTISRDNVKN
SLQLQMNSLRVEDTAVYYCARDEFPYGMDVWGQGTTVTVSSGGGGSGGGG
SAHPGRPRAVPTQCDVPPNSRFDCAPDKAITQEQCEARGCCYIPAKQGLQ
GAQMGQPWCFFPPSYPSYKLENLSSSEMGYTATLTRTTPTFFPKDILTLR
LDVMMETENRLHFTIKDPANRRYEVPLETPHVHSRAPSPLYSVEFSEEPF
GVIVRRQLDGRVLLNTTVAPLFFADQFLQLSTSLPSQYITGLAEHLSPLM
LSTSWTRITLWNRDLAPTPGANLYGSHPFYLALEDGGSAHGVFLLNSNAM
DVVLQPSPALSWRSTGGILDVYIFLGPEPKSVVQQYLDVVGYPFMPPYWG
LGFHLCRWGYSSTAITRQVVENMTRAHFPLDVQWNDLDYMDSRRDFTFNK
DGFRDFPAMVQELHQGGRRYMMIVDPAISSSGPAGSYRPYDEGLRRGVFI
TNETGQPLIGKVWPGSTAFPDFTNPTALAWWEDMVAEFHDQVPFDGMWID
MNEPSNFIRGSEDGCPNNELENPPYVPGVVGGTLQAATICASSHQFLSTH
YNLHNLYGLTEAIASHRALVKARGTRPFVISRSTFAGHGRYAGHWTGDVW
SSWEQLASSVPEILQFNLLGVPLVGADVCGFLGNTSEELCVRWTQLGAFY
PFMRNHNSLLSLPQEPYSFSEPAQQAMRKALTLRYALLPHLYTLFHQAHV
AGETVARPLFLEFPKDSSTWTVDHQLLWGEALLITPVLQAGKAEVTGYFP
LGTWYDLQTVPVEALGSLPPPPAAPREPAIHSEGQWVTLPAPLDTINVHL
RAGYIIPLQGPGLTTTESRQQPMALAVALTKGGEARGELFWDDGESLEVL
ERGAYTQVIFLARNNTIVNELVRVTSEGAGLQLQKVTVLGVATAPQQVLS
NGVPVSNFTYSPDTKVLDICVSLLMGEQFLVSWC(SEQ ID NO: 684)

12839B (REGN16822)

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPITFG
QGTRLEIKGGGGSGGGGSGGGGSQVQLVESGGGVVQPGRSLRLSCAASGF
PFSNYVMYWVRQAPGKGLEWVALIFFDGKKNYHADSVKGRFTITRDNSKN
MLYLQMNSLRPEDAAVYYCAKIHCPNGVCYKGYYGMDVWGQGTTVTVSSG
GGGSGGGGSAHPGRPRAVPTQCDVPPNSRFDCAPDKAITQEQCEARGCCY
IPAKQGLQGAQMGQPWCFFPPSYPSYKLENLSSSEMGYTATLTRTTPTFF
PKDILTLRLDVMMETENRLHFTIKDPANRRYEVPLETPHVHSRAPSPLYS
VEFSEEPFGVIVRRQLDGRVLLNTTVAPLFFADQFLQLSTSLPSQYITGL
AEHLSPLMLSTSWTRITLWNRDLAPTPGANLYGSHPFYLALEDGGSAHGV
FLLNSNAMDVVLQPSPALSWRSTGGILDVYIFLGPEPKSVVQQYLDVVGY
PFMPPYWGLGFHLCRWGYSSTAITRQVVENMTRAHFPLDVQWNDLDYMDS
RRDFTFNKDGFRDFPAMVQELHQGGRRYMMIVDPAISSSGPAGSYRPYDE
GLRRGVFITNETGQPLIGKVWPGSTAFPDFTNPTALAWWEDMVAEFHDQV
PFDGMWIDMNEPSNFIRGSEDGCPNNELENPPYVPGVVGGTLQAATICAS
SHQFLSTHYNLHNLYGLTEAIASHRALVKARGTRPFVISRSTFAGHGRYA
GHWTGDVWSSWEQLASSVPEILQFNLLGVPLVGADVCGFLGNTSEELCVR
WTQLGAFYPFMRNHNSLLSLPQEPYSFSEPAQQAMRKALTLRYALLPHLY
TLFHQAHVAGETVARPLFLEFPKDSSTWTVDHQLLWGEALLITPVLQAGK
AEVTGYFPLGTWYDLQTVPVEALGSLPPPPAAPREPAIHSEGQWVTLPAP
LDTINVHLRAGYIIPLQGPGLTTTESRQQPMALAVALTKGGEARGELFWD
DGESLEVLERGAYTQVIFLARNNTIVNELVRVTSEGAGLQLQKVTVLGVA
TAPQQVLSNGVPVSNFTYSPDTKVLDICVSLLMGEQFLVSWC(SEQ ID
NO: 571)

12841B (REGN16823)

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPITFG
QGTRLEIKGGGGSGGGGSGGGGSEVQLVESGGGLVQPGGSLRLSCAASGF
TFSNYWMNWVRQAPGKGLEWVANIKEDGGKKLYVDSVKGRFTISRDNAKN
SLFLQMNSLRAEDTAVYYCAREDTTLVVDYYYYGMDVWGQGTTVTVSSGG
GGSGGGGSAHPGRPRAVPTQCDVPPNSRFDCAPDKAITQEQCEARGCCYI
PAKQGLQGAQMGQPWCFFPPSYPSYKLENLSSSEMGYTATLTRTTPTFFP
KDILTLRLDVMMETENRLHFTIKDPANRRYEVPLETPHVHSRAPSPLYSV
EFSEEPFGVIVRRQLDGRVLLNTTVAPLFFADQFLQLSTSLPSQYITGLA
EHLSPLMLSTSWTRITLWNRDLAPTPGANLYGSHPFYLALEDGGSAHGVF
LLNSNAMDVVLQPSPALSWRSTGGILDVYIFLGPEPKSVVQQYLDVVGYP
FMPPYWGLGFHLCRWGYSSTAITRQVVENMTRAHFPLDVQWNDLDYMDSR
RDFTFNKDGFRDFPAMVQELHQGGRRYMMIVDPAISSSGPAGSYRPYDEG
LRRGVFITNETGQPLIGKVWPGSTAFPDFTNPTALAWWEDMVAEFHDQVP
FDGMWIDMNEPSNFIRGSEDGCPNNELENPPYVPGVVGGTLQAATICASS
HQFLSTHYNLHNLYGLTEAIASHRALVKARGTRPFVISRSTFAGHGRYAG
HWTGDVWSSWEQLASSVPEILQFNLLGVPLVGADVCGFLGNTSEELCVRW
TQLGAFYPFMRNHNSLLSLPQEPYSFSEPAQQAMRKALTLRYALLPHLYT
LFHQAHVAGETVARPLFLEFPKDSSTWTVDHQLLWGEALLITPVLQAGKA
EVTGYFPLGTWYDLQTVPVEALGSLPPPPAAPREPAIHSEGQWVTLPAPL
DTINVHLRAGYIIPLQGPGLTTTESRQQPMALAVALTKGGEARGELFWDD
GESLEVLERGAYTQVIFLARNNTIVNELVRVTSEGAGLQLQKVTVLGVAT
APQQVLSNGVPVSNFTYSPDTKVLDICVSLLMGEQFLVSWC(SEQ ID N
O: 685)

12843B (REGN16824)

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPITFG
QGTRLEIKGGGGSGGGGSGGGGSEVQLVESGGGLVQPGGSLRLSCAASGF
TFNIFEMNWVRQAPGKGLEWISYISSRGTTTYYADSVRGRFTISRDNAKN
SLYLQMNSLRAEDTAVYYCARDYEATIPFDFWGQGTLVTVSSGGGGSGGG
GSAHPGRPRAVPTQCDVPPNSRFDCAPDKAITQEQCEARGCCYIPAKQGL
QGAQMGQPWCFFPPSYPSYKLENLSSSEMGYTATLTRTTPTFFPKDILTL
RLDVMMETENRLHFTIKDPANRRYEVPLETPHVHSRAPSPLYSVEFSEEP
FGVIVRRQLDGRVLLNTTVAPLFFADQFLQLSTSLPSQYITGLAEHLSPL
MLSTSWTRITLWNRDLAPTPGANLYGSHPFYLALEDGGSAHGVFLLNSNA
MDVVLQPSPALSWRSTGGILDVYIFLGPEPKSVVQQYLDVVGYPFMPPYW
GLGFHLCRWGYSSTAITRQVVENMTRAHFPLDVQWNDLDYMDSRRDFTFN
KDGFRDFPAMVQELHQGGRRYMMIVDPAISSSGPAGSYRPYDEGLRRGVF
ITNETGQPLIGKVWPGSTAFPDFTNPTALAWWEDMVAEFHDQVPFDGMWI
DMNEPSNFIRGSEDGCPNNELENPPYVPGVVGGTLQAATICASSHQFLST
HYNLHNLYGLTEAIASHRALVKARGTRPFVISRSTFAGHGRYAGHWTGDV
WSSWEQLASSVPEILQFNLLGVPLVGADVCGFLGNTSEELCVRWTQLGAF
YPFMRNHNSLLSLPQEPYSFSEPAQQAMRKALTLRYALLPHLYTLFHQAH
VAGETVARPLFLEFPKDSSTWTVDHQLLWGEALLITPVLQAGKAEVTGYF
PLGTWYDLQTVPVEALGSLPPPPAAPREPAIHSEGQWVTLPAPLDTINVH
LRAGYIIPLQGPGLTTTESRQQPMALAVALTKGGEARGELFWDDGESLEV
LERGAYTQVIFLARNNTIVNELVRVTSEGAGLQLQKVTVLGVATAPQQVL
SNGVPVSNFTYSPDTKVLDICVSLLMGEQFLVSWC(SEQ ID NO: 572
)

12844B

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPITFG
QGTRLEIKGGGGSGGGGSGGGGSEVQLVESGGSVVRPGGSLRLSCEASGF
TFDDYGMSWVRQDPGKGLEWVSGINWNGDRTNYADSVKGRFIISRDNAKN
SVYLQMNSLRAEDSALYHCARDQGLGVAATLDYWGQGTLVTVSSGGGGSG
GGGSAHPGRPRAVPTQCDVPPNSRFDCAPDKAITQEQCEARGCCYIPAKQ
GLQGAQMGQPWCFFPPSYPSYKLENLSSSEMGYTATLTRTTPTFFPKDIL
TLRLDVMMETENRLHFTIKDPANRRYEVPLETPHVHSRAPSPLYSVEFSE
EPFGVIVRRQLDGRVLLNTTVAPLFFADQFLQLSTSLPSQYITGLAEHLS
PLMLSTSWTRITLWNRDLAPTPGANLYGSHPFYLALEDGGSAHGVFLLNS
NAMDVVLQPSPALSWRSTGGILDVYIFLGPEPKSVVQQYLDVVGYPFMPP
YWGLGFHLCRWGYSSTAITRQVVENMTRAHFPLDVQWNDLDYMDSRRDFT
FNKDGFRDFPAMVQELHQGGRRYMMIVDPAISSSGPAGSYRPYDEGLRRG
VFITNETGQPLIGKVWPGSTAFPDFTNPTALAWWEDMVAEFHDQVPFDGM
WIDMNEPSNFIRGSEDGCPNNELENPPYVPGVVGGTLQAATICASSHQFL
STHYNLHNLYGLTEAIASHRALVKARGTRPFVISRSTFAGHGRYAGHWTG
DVWSSWEQLASSVPEILQFNLLGVPLVGADVCGFLGNTSEELCVRWTQLG
AFYPFMRNHNSLLSLPQEPYSFSEPAQQAMRKALTLRYALLPHLYTLFHQ
AHVAGETVARPLFLEFPKDSSTWTVDHQLLWGEALLITPVLQAGKAEVTG
YFPLGTWYDLQTVPVEALGSLPPPPAAPREPAIHSEGQWVTLPAPLDTIN
VHLRAGYIIPLQGPGLTTTESRQQPMALAVALTKGGEARGELFWDDGESL
EVLERGAYTQVIFLARNNTIVNELVRVTSEGAGLQLQKVTVLGVATAPQQ
VLSNGVPVSNFTYSPDTKVLDICVSLLMGEQFLVSWC(SEQ ID NO: 6
86)

12845B (REGN16825)

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPITFG
QGTRLEIKGGGGSGGGGSGGGGSEVQLVESGGGLVQPGGSLRLSCAASGF
TVSNYEMNWVRQAPGKGLEWVSYISSSTSNIYYADSVKGRFTISRDNAEN
SLYLQMNSLRVEDTAVYYCVRDGIVVVPVGRGYYYYGLDVWGQGTTVTVS
SGGGGSGGGGSAHPGRPRAVPTQCDVPPNSRFDCAPDKAITQEQCEARGC
CYIPAKQGLQGAQMGQPWCFFPPSYPSYKLENLSSSEMGYTATLTRTTPT
FFPKDILTLRLDVMMETENRLHFTIKDPANRRYEVPLETPHVHSRAPSPL
YSVEFSEEPFGVIVRRQLDGRVLLNTTVAPLFFADQFLQLSTSLPSQYIT
GLAEHLSPLMLSTSWTRITLWNRDLAPTPGANLYGSHPFYLALEDGGSAH
GVFLLNSNAMDVVLQPSPALSWRSTGGILDVYIFLGPEPKSVVQQYLDVV
GYPFMPPYWGLGFHLCRWGYSSTAITRQVVENMTRAHFPLDVQWNDLDYM
DSRRDFTFNKDGFRDFPAMVQELHQGGRRYMMIVDPAISSSGPAGSYRPY
DEGLRRGVFITNETGQPLIGKVWPGSTAFPDFTNPTALAWWEDMVAEFHD
QVPFDGMWIDMNEPSNFIRGSEDGCPNNELENPPYVPGVVGGTLQAATIC
ASSHQFLSTHYNLHNLYGLTEAIASHRALVKARGTRPFVISRSTFAGHGR
YAGHWTGDVWSSWEQLASSVPEILQFNLLGVPLVGADVCGFLGNTSEELC
VRWTQLGAFYPFMRNHNSLLSLPQEPYSFSEPAQQAMRKALTLRYALLPH
LYTLFHQAHVAGETVARPLFLEFPKDSSTWTVDHQLLWGEALLITPVLQA
GKAEVTGYFPLGTWYDLQTVPVEALGSLPPPPAAPREPAIHSEGQWVTLP
APLDTINVHLRAGYIIPLQGPGLTTTESRQQPMALAVALTKGGEARGELF
WDDGESLEVLERGAYTQVIFLARNNTIVNELVRVTSEGAGLQLQKVTVLG
VATAPQQVLSNGVPVSNFTYSPDTKVLDICVSLLMGEQFLVSWC(SEQ I
D NO: 687)

12847B (REGN16826)

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPITFG
QGTRLEIKGGGGSGGGGSGGGGSEVQLVESGGGLVQPGRSLRLSCAASGF
TFDDYAMNWVRQAPGKGLEWVSGISWSSGSMDYADSVKGRFTISRDNAKN
SLYLQMNSLRTEDTALYYCAKAREVGDYYGMDVWGQGTTVTVSSGGGGSG
GGGSAHPGRPRAVPTQCDVPPNSRFDCAPDKAITQEQCEARGCCYIPAKQ
GLQGAQMGQPWCFFPPSYPSYKLENLSSSEMGYTATLTRTTPTFFPKDIL
TLRLDVMMETENRLHFTIKDPANRRYEVPLETPHVHSRAPSPLYSVEFSE
EPFGVIVRRQLDGRVLLNTTVAPLFFADQFLQLSTSLPSQYITGLAEHLS
PLMLSTSWTRITLWNRDLAPTPGANLYGSHPFYLALEDGGSAHGVFLLNS
NAMDVVLQPSPALSWRSTGGILDVYIFLGPEPKSVVQQYLDVVGYPFMPP
YWGLGFHLCRWGYSSTAITRQVVENMTRAHFPLDVQWNDLDYMDSRRDFT
FNKDGFRDFPAMVQELHQGGRRYMMIVDPAISSSGPAGSYRPYDEGLRRG
VFITNETGQPLIGKVWPGSTAFPDFTNPTALAWWEDMVAEFHDQVPFDGM
WIDMNEPSNFIRGSEDGCPNNELENPPYVPGVVGGTLQAATICASSHQFL
STHYNLHNLYGLTEAIASHRALVKARGTRPFVISRSTFAGHGRYAGHWTG
DVWSSWEQLASSVPEILQFNLLGVPLVGADVCGFLGNTSEELCVRWTQLG
AFYPFMRNHNSLLSLPQEPYSFSEPAQQAMRKALTLRYALLPHLYTLFHQ
AHVAGETVARPLFLEFPKDSSTWTVDHQLLWGEALLITPVLQAGKAEVTG
YFPLGTWYDLQTVPVEALGSLPPPPAAPREPAIHSEGQWVTLPAPLDTIN
VHLRAGYIIPLQGPGLTTTESRQQPMALAVALTKGGEARGELFWDDGESL
EVLERGAYTQVIFLARNNTIVNELVRVTSEGAGLQLQKVTVLGVATAPQQ
VLSNGVPVSNFTYSPDTKVLDICVSLLMGEQFLVSWC(SEQ ID NO: 5
73)

12848B (REGN16827)

EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPWTFG
QGTKVEIKGGGGSGGGGSGGGGSEVQLVESGGGLVQPGRSLTLSCAASGF
TFDNFGMHWVRQGPGKGLEWVSGLTWNSGVIGYADSVKGRFTISRDNAKN
SLYLQMNSLRPEDTALYYCAKDIRNYGPFDYWGQGTLVTVSSGGGGSGGG
GSAHPGRPRAVPTQCDVPPNSRFDCAPDKAITQEQCEARGCCYIPAKQGL
QGAQMGQPWCFFPPSYPSYKLENLSSSEMGYTATLTRTTPTFFPKDILTL
RLDVMMETENRLHFTIKDPANRRYEVPLETPHVHSRAPSPLYSVEFSEEP
FGVIVRRQLDGRVLLNTTVAPLFFADQFLQLSTSLPSQYITGLAEHLSPL
MLSTSWTRITLWNRDLAPTPGANLYGSHPFYLALEDGGSAHGVFLLNSNA
MDVVLQPSPALSWRSTGGILDVYIFLGPEPKSVVQQYLDVVGYPFMPPYW
GLGFHLCRWGYSSTAITRQVVENMTRAHFPLDVQWNDLDYMDSRRDFTFN
KDGFRDFPAMVQELHQGGRRYMMIVDPAISSSGPAGSYRPYDEGLRRGVF
ITNETGQPLIGKVWPGSTAFPDFTNPTALAWWEDMVAEFHDQVPFDGMWI
DMNEPSNFIRGSEDGCPNNELENPPYVPGVVGGTLQAATICASSHQFLST
HYNLHNLYGLTEAIASHRALVKARGTRPFVISRSTFAGHGRYAGHWTGDV
WSSWEQLASSVPEILQFNLLGVPLVGADVCGFLGNTSEELCVRWTQLGAF
YPFMRNHNSLLSLPQEPYSFSEPAQQAMRKALTLRYALLPHLYTLFHQAH
VAGETVARPLFLEFPKDSSTWTVDHQLLWGEALLITPVLQAGKAEVTGYF
PLGTWYDLQTVPVEALGSLPPPPAAPREPAIHSEGQWVTLPAPLDTINVH
LRAGYIIPLQGPGLTTTESRQQPMALAVALTKGGEARGELFWDDGESLEV
LERGAYTQVIFLARNNTIVNELVRVTSEGAGLQLQKVTVLGVATAPQQVL
SNGVPVSNFTYSPDTKVLDICVSLLMGEQFLVSWC(SEQ ID NO: 688
)

12850B (REGN16828)

EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPWTFG
QGTKVEIKGGGGSGGGGSGGGGSQVQLVQSGAEVKKPGSSVKVSCKASGG
TFNTYAITWVRQAPGQGLEWMGGIIPISGIAEYAQKFQGRVTITTDDSST
TAYMELNSLRSEDTAVYYCASWNYALYYFYGMDVWGRGTTVTVSSGGGGS
GGGGSAHPGRPRAVPTQCDVPPNSRFDCAPDKAITQEQCEARGCCYIPAK
QGLQGAQMGQPWCFFPPSYPSYKLENLSSSEMGYTATLTRTTPTFFPKDI
LTLRLDVMMETENRLHFTIKDPANRRYEVPLETPHVHSRAPSPLYSVEFS
EEPFGVIVRRQLDGRVLLNTTVAPLFFADQFLQLSTSLPSQYITGLAEHL
SPLMLSTSWTRITLWNRDLAPTPGANLYGSHPFYLALEDGGSAHGVFLLN
SNAMDVVLQPSPALSWRSTGGILDVYIFLGPEPKSVVQQYLDVVGYPFMP
PYWGLGFHLCRWGYSSTAITRQVVENMTRAHFPLDVQWNDLDYMDSRRDF
TFNKDGFRDFPAMVQELHQGGRRYMMIVDPAISSSGPAGSYRPYDEGLRR
GVFITNETGQPLIGKVWPGSTAFPDFTNPTALAWWEDMVAEFHDQVPFDG
MWIDMNEPSNFIRGSEDGCPNNELENPPYVPGVVGGTLQAATICASSHQF
LSTHYNLHNLYGLTEAIASHRALVKARGTRPFVISRSTFAGHGRYAGHWT
GDVWSSWEQLASSVPEILQFNLLGVPLVGADVCGFLGNTSEELCVRWTQL
GAFYPFMRNHNSLLSLPQEPYSFSEPAQQAMRKALTLRYALLPHLYTLFH
QAHVAGETVARPLFLEFPKDSSTWTVDHQLLWGEALLITPVLQAGKAEVT
GYFPLGTWYDLQTVPVEALGSLPPPPAAPREPAIHSEGQWVTLPAPLDTI
NVHLRAGYIIPLQGPGLTTTESRQQPMALAVALTKGGEARGELFWDDGES
LEVLERGAYTQVIFLARNNTIVNELVRVTSEGAGLQLQKVTVLGVATAPQ
QVLSNGVPVSNFTYSPDTKVLDICVSLLMGEQFLVSWC(SEQ ID NO:
689)

31863B

DIQMTQSPSSLSASIGDRVTITCRASQGISNYLAWYQQKPGKVPKLLIYA
ASTLQSGVPSRFSGSGSGTDFTLTISSLQPEDVATYYCQNHNSVPLTFGG
GTKVEIKGGGGSGGGGSGGGGSEVQLVESGGGLVQPGGSLRLSCAASGFT
FNSYAMTWVRQAPGKGLEWVSFIGGSTGNTYYAGSVKGRFTISSDNSKKT
LYLQMNSLRAEDTAVYYCAKGGAARRMEYFQHWGQGTLVTVSSGGGGSGG
GGSAHPGRPRAVPTQCDVPPNSRFDCAPDKAITQEQCEARGCCYIPAKQG
LQGAQMGQPWCFFPPSYPSYKLENLSSSEMGYTATLTRTTPTFFPKDILT
LRLDVMMETENRLHFTIKDPANRRYEVPLETPHVHSRAPSPLYSVEFSEE
PFGVIVRRQLDGRVLLNTTVAPLFFADQFLQLSTSLPSQYITGLAEHLSP
LMLSTSWTRITLWNRDLAPTPGANLYGSHPFYLALEDGGSAHGVFLLNSN
AMDVVLQPSPALSWRSTGGILDVYIFLGPEPKSVVQQYLDVVGYPFMPPY
WGLGFHLCRWGYSSTAITRQVVENMTRAHFPLDVQWNDLDYMDSRRDFTF
NKDGFRDFPAMVQELHQGGRRYMMIVDPAISSSGPAGSYRPYDEGLRRGV
FITNETGQPLIGKVWPGSTAFPDFTNPTALAWWEDMVAEFHDQVPFDGMW
IDMNEPSNFIRGSEDGCPNNELENPPYVPGVVGGTLQAATICASSHQFLS
THYNLHNLYGLTEAIASHRALVKARGTRPFVISRSTFAGHGRYAGHWTGD
VWSSWEQLASSVPEILQFNLLGVPLVGADVCGFLGNTSEELCVRWTQLGA
FYPFMRNHNSLLSLPQEPYSFSEPAQQAMRKALTLRYALLPHLYTLFHQA
HVAGETVARPLFLEFPKDSSTWTVDHQLLWGEALLITPVLQAGKAEVTGY
FPLGTWYDLQTVPVEALGSLPPPPAAPREPAIHSEGQWVTLPAPLDTINV
HLRAGYIIPLQGPGLTTTESRQQPMALAVALTKGGEARGELFWDDGESLE
VLERGAYTQVIFLARNNTIVNELVRVTSEGAGLQLQKVTVLGVATAPQQV
LSNGVPVSNFTYSPDTKVLDICVSLLMGEQFLVSWC(SEQ ID NO: 69
0)

31874B

DIQMTQSPSSLSASVGDRVTITCRASQGISNYLAWYQQKPGKVPNLLIYA
ASTLQSGVPSRFSGSGSGTDFTLTISSLQPEDVATYYCQKYNSAPLTFGG
GTKVEIKGGGGSGGGGSGGGGSEVQLVESGGGLVQPGGSLRLSCAASGFA
FSSYAMTWVRQAPGKGLEWVSVISGTGGSTYYADSVKGRFTISRDNSKNT
LYLQMNSLRAEDTAVYYCAKGGAARRMEYFQYWGQGTLVTVSSGGGGSGG
GGSAHPGRPRAVPTQCDVPPNSRFDCAPDKAITQEQCEARGCCYIPAKQG
LQGAQMGQPWCFFPPSYPSYKLENLSSSEMGYTATLTRTTPTFFPKDILT
LRLDVMMETENRLHFTIKDPANRRYEVPLETPHVHSRAPSPLYSVEFSEE
PFGVIVRRQLDGRVLLNTTVAPLFFADQFLQLSTSLPSQYITGLAEHLSP
LMLSTSWTRITLWNRDLAPTPGANLYGSHPFYLALEDGGSAHGVFLLNSN
AMDVVLQPSPALSWRSTGGILDVYIFLGPEPKSVVQQYLDVVGYPFMPPY
WGLGFHLCRWGYSSTAITRQVVENMTRAHFPLDVQWNDLDYMDSRRDFTF
NKDGFRDFPAMVQELHQGGRRYMMIVDPAISSSGPAGSYRPYDEGLRRGV
FITNETGQPLIGKVWPGSTAFPDFTNPTALAWWEDMVAEFHDQVPFDGMW
IDMNEPSNFIRGSEDGCPNNELENPPYVPGVVGGTLQAATICASSHQFLS
THYNLHNLYGLTEAIASHRALVKARGTRPFVISRSTFAGHGRYAGHWTGD
VWSSWEQLASSVPEILQFNLLGVPLVGADVCGFLGNTSEELCVRWTQLGA
FYPFMRNHNSLLSLPQEPYSFSEPAQQAMRKALTLRYALLPHLYTLFHQA
HVAGETVARPLFLEFPKDSSTWTVDHQLLWGEALLITPVLQAGKAEVTGY
FPLGTWYDLQTVPVEALGSLPPPPAAPREPAIHSEGQWVTLPAPLDTINV
HLRAGYIIPLQGPGLTTTESRQQPMALAVALTKGGEARGELFWDDGESLE
VLERGAYTQVIFLARNNTIVNELVRVTSEGAGLQLQKVTVLGVATAPQQV
LSNGVPVSNFTYSPDTKVLDICVSLLMGEQFLVSWC(SEQ ID NO: 69
1)

69261

DIVMTQSPLSLPVTPGEPASISCRSSQSLLHSNGYNYLDWYLQKPGQSPQ
FLIYLGSNRASGVPDRFSGSGSGTDFTLKINRVEAEDVGVYYCMQALQTP
YTFGQGTKLEIKGGGGSGGGGSGGGGSQVQLVESGGGLVKPGGSLRLSCA
ASGFTFSVYYMNWIRQAPGKGLEWVSYISSSGSTIYYADSVKGRFTISRD
NAKNSLYLQMNSLRAEDTAVYYCGREGYSGTYSYYGMDVWGQGTTVTVSS
GGGGSGGGGSAHPGRPRAVPTQCDVPPNSRFDCAPDKAITQEQCEARGCC
YIPAKQGLQGAQMGQPWCFFPPSYPSYKLENLSSSEMGYTATLTRTTPTF
FPKDILTLRLDVMMETENRLHFTIKDPANRRYEVPLETPHVHSRAPSPLY
SVEFSEEPFGVIVRRQLDGRVLLNTTVAPLFFADQFLQLSTSLPSQYITG
LAEHLSPLMLSTSWTRITLWNRDLAPTPGANLYGSHPFYLALEDGGSAHG
VFLLNSNAMDVVLQPSPALSWRSTGGILDVYIFLGPEPKSVVQQYLDVVG
YPFMPPYWGLGFHLCRWGYSSTAITRQVVENMTRAHFPLDVQWNDLDYMD
SRRDFTFNKDGFRDFPAMVQELHQGGRRYMMIVDPAISSSGPAGSYRPYD
EGLRRGVFITNETGQPLIGKVWPGSTAFPDFTNPTALAWWEDMVAEFHDQ
VPFDGMWIDMNEPSNFIRGSEDGCPNNELENPPYVPGVVGGTLQAATICA
SSHQFLSTHYNLHNLYGLTEAIASHRALVKARGTRPFVISRSTFAGHGRY
AGHWTGDVWSSWEQLASSVPEILQFNLLGVPLVGADVCGFLGNTSEELCV
RWTQLGAFYPFMRNHNSLLSLPQEPYSFSEPAQQAMRKALTLRYALLPHL
YTLFHQAHVAGETVARPLFLEFPKDSSTWTVDHQLLWGEALLITPVLQAG
KAEVTGYFPLGTWYDLQTVPVEALGSLPPPPAAPREPAIHSEGQWVTLPA
PLDTINVHLRAGYIIPLQGPGLTTTESRQQPMALAVALTKGGEARGELFW
DDGESLEVLERGAYTQVIFLARNNTIVNELVRVTSEGAGLQLQKVTVLGV
ATAPQQVLSNGVPVSNFTYSPDTKVLDICVSLLMGEQFLVSWC(SEQ ID
NO: 692)

69263

DIQMTQSPSSLSASVGDRVTITCRASQDISHYSAWYQQKPGKLPNLLIYA
ASTLQSGVPSRFSGSGSGTDFSLTTSSLQPEDVATYYCQKYNSVPLTFGG
GTKVEIKGGGGSGGGGSGGGGSEVQLVESGGGLVQPGRSLRLSCAVSGFT
FDDYAMHWVRQAPGKGLEWVSGISWNSGTRGYADSVKGRFTISRDNAKNS
LYLQMNSLRGEDTALYYCVKDITISPNYYGMDVWGQGTTVTVSSGGGGSG
GGGSAHPGRPRAVPTQCDVPPNSRFDCAPDKAITQEQCEARGCCYIPAKQ
GLQGAQMGQPWCFFPPSYPSYKLENLSSSEMGYTATLTRTTPTFFPKDIL
TLRLDVMMETENRLHFTIKDPANRRYEVPLETPHVHSRAPSPLYSVEFSE
EPFGVIVRRQLDGRVLLNTTVAPLFFADQFLQLSTSLPSQYITGLAEHLS
PLMLSTSWTRITLWNRDLAPTPGANLYGSHPFYLALEDGGSAHGVFLLNS
NAMDVVLQPSPALSWRSTGGILDVYIFLGPEPKSVVQQYLDVVGYPFMPP
YWGLGFHLCRWGYSSTAITRQVVENMTRAHFPLDVQWNDLDYMDSRRDFT
FNKDGFRDFPAMVQELHQGGRRYMMIVDPAISSSGPAGSYRPYDEGLRRG
VFITNETGQPLIGKVWPGSTAFPDFTNPTALAWWEDMVAEFHDQVPFDGM
WIDMNEPSNFIRGSEDGCPNNELENPPYVPGVVGGTLQAATICASSHQFL
STHYNLHNLYGLTEAIASHRALVKARGTRPFVISRSTFAGHGRYAGHWTG
DVWSSWEQLASSVPEILQFNLLGVPLVGADVCGFLGNTSEELCVRWTQLG
AFYPFMRNHNSLLSLPQEPYSFSEPAQQAMRKALTLRYALLPHLYTLFHQ
AHVAGETVARPLFLEFPKDSSTWTVDHQLLWGEALLITPVLQAGKAEVTG
YFPLGTWYDLQTVPVEALGSLPPPPAAPREPAIHSEGQWVTLPAPLDTIN
VHLRAGYIIPLQGPGLTTTESRQQPMALAVALTKGGEARGELFWDDGESL
EVLERGAYTQVIFLARNNTIVNELVRVTSEGAGLQLQKVTVLGVATAPQQ
VLSNGVPVSNFTYSPDTKVLDICVSLLMGEQFLVSWC(SEQ ID NO: 6
93)

69305

DIQMTQSPSSLSASVGDRVTITCRASQSIDRYLNWYRQKPGKAPKLLIYT
TSSLQSGVPSRFSGSGSGTDFTLTLSSLQPEDFATYYCQQSYSPPLTFGG
GTKVEIKGGGGSGGGGSGGGGSQVQLVESGGGVVQPGRSLRLSCAASGFT
FSSYGMHWVRQAPGKGLEWVAVIWYDGSNKYYADSVKGRFTISRDISKNT
LYLQMNSLRAEDTAVYYCAGQLDLFFDYWGQGTLVTVSSGGGGSGGGGSA
HPGRPRAVPTQCDVPPNSRFDCAPDKAITQEQCEARGCCYIPAKQGLQGA
QMGQPWCFFPPSYPSYKLENLSSSEMGYTATLTRTTPTFFPKDILTLRLD
VMMETENRLHFTIKDPANRRYEVPLETPHVHSRAPSPLYSVEFSEEPFGV
IVRRQLDGRVLLNTTVAPLFFADQFLQLSTSLPSQYITGLAEHLSPLMLS
TSWTRITLWNRDLAPTPGANLYGSHPFYLALEDGGSAHGVFLLNSNAMDV
VLQPSPALSWRSTGGILDVYIFLGPEPKSVVQQYLDVVGYPFMPPYWGLG
FHLCRWGYSSTAITRQVVENMTRAHFPLDVQWNDLDYMDSRRDFTFNKDG
FRDFPAMVQELHQGGRRYMMIVDPAISSSGPAGSYRPYDEGLRRGVFITN
ETGQPLIGKVWPGSTAFPDFTNPTALAWWEDMVAEFHDQVPFDGMWIDMN
EPSNFIRGSEDGCPNNELENPPYVPGVVGGTLQAATICASSHQFLSTHYN
LHNLYGLTEAIASHRALVKARGTRPFVISRSTFAGHGRYAGHWTGDVWSS
WEQLASSVPEILQFNLLGVPLVGADVCGFLGNTSEELCVRWTQLGAFYPF
MRNHNSLLSLPQEPYSFSEPAQQAMRKALTLRYALLPHLYTLFHQAHVAG
ETVARPLFLEFPKDSSTWTVDHQLLWGEALLITPVLQAGKAEVTGYFPLG
TWYDLQTVPVEALGSLPPPPAAPREPAIHSEGQWVTLPAPLDTINVHLRA
GYIIPLQGPGLTTTESRQQPMALAVALTKGGEARGELFWDDGESLEVLER
GAYTQVIFLARNNTIVNELVRVTSEGAGLQLQKVTVLGVATAPQQVLSNG
VPVSNFTYSPDTKVLDICVSLLMGEQFLVSWC(SEQ ID NO: 694)

69307 (REGN16817)

DIQMTQSPSSVSASVGDRVTITCRASQGISSWLAWYQQKPGKAPKLLIYA
ASSLQSGVPSRFSGSGSGTDFTLTISSLQPEDFATYYCQKADSLPYAFGQ
GTKLEIKGGGGSGGGGSGGGGSEVQLVESGGGLVQPGGSLRLSCTASGFT
FSNYWMTWVRQAPGKGLEWVANIKEDGSEKEYVDSVKGRFTISRDNAKNS
LYLQMNSLRGEDTAVYYCARDGEQLVDYYYYYVMDVWGQGTTVTVSSGGG
GSGGGGSAHPGRPRAVPTQCDVPPNSRFDCAPDKAITQEQCEARGCCYIP
AKQGLQGAQMGQPWCFFPPSYPSYKLENLSSSEMGYTATLTRTTPTFFPK
DILTLRLDVMMETENRLHFTIKDPANRRYEVPLETPHVHSRAPSPLYSVE
FSEEPFGVIVRRQLDGRVLLNTTVAPLFFADQFLQLSTSLPSQYITGLAE
HLSPLMLSTSWTRITLWNRDLAPTPGANLYGSHPFYLALEDGGSAHGVFL
LNSNAMDVVLQPSPALSWRSTGGILDVYIFLGPEPKSVVQQYLDVVGYPF
MPPYWGLGFHLCRWGYSSTAITRQVVENMTRAHFPLDVQWNDLDYMDSRR
DFTFNKDGFRDFPAMVQELHQGGRRYMMIVDPAISSSGPAGSYRPYDEGL
RRGVFITNETGQPLIGKVWPGSTAFPDFTNPTALAWWEDMVAEFHDQVPF
DGMWIDMNEPSNFIRGSEDGCPNNELENPPYVPGVVGGTLQAATICASSH
QFLSTHYNLHNLYGLTEAIASHRALVKARGTRPFVISRSTFAGHGRYAGH
WTGDVWSSWEQLASSVPEILQFNLLGVPLVGADVCGFLGNTSEELCVRWT
QLGAFYPFMRNHNSLLSLPQEPYSFSEPAQQAMRKALTLRYALLPHLYTL
FHQAHVAGETVARPLFLEFPKDSSTWTVDHQLLWGEALLITPVLQAGKAE
VTGYFPLGTWYDLQTVPVEALGSLPPPPAAPREPAIHSEGQWVTLPAPLD
TINVHLRAGYIIPLQGPGLTTTESRQQPMALAVALTKGGEARGELFWDDG
ESLEVLERGAYTQVIFLARNNTIVNELVRVTSEGAGLQLQKVTVLGVATA
PQQVLSNGVPVSNFTYSPDTKVLDICVSLLMGEQFLVSWC(SEQ ID NO
: 695)

69323 (REGN16816)

DIQMTQSPSSLSASVGDRVTITCRASQSISSYLNWYQQKPGKAPKVLIYA
ASSLQSGVPSRFSGSGSGTDFTLTISSLQPEDFATYYCQQSYSIPLTFGG
GTKVEIKGGGGSGGGGSGGGGSEVQLVESGGGLVQPGRSLRLSCAASGFT
FDDYAMHWVRQAPGKGLEWVSGISWNSGYIGYADSVKGRFTISRDNAENS
LHLQMNSLRAEDTALYYCARGGSTLVRGVKGGYYGMDVWGQGTTVTVSSG
GGGSGGGGSAHPGRPRAVPTQCDVPPNSRFDCAPDKAITQEQCEARGCCY
IPAKQGLQGAQMGQPWCFFPPSYPSYKLENLSSSEMGYTATLTRTTPTFF
PKDILTLRLDVMMETENRLHFTIKDPANRRYEVPLETPHVHSRAPSPLYS
VEFSEEPFGVIVRRQLDGRVLLNTTVAPLFFADQFLQLSTSLPSQYITGL
AEHLSPLMLSTSWTRITLWNRDLAPTPGANLYGSHPFYLALEDGGSAHGV
FLLNSNAMDVVLQPSPALSWRSTGGILDVYIFLGPEPKSVVQQYLDVVGY
PFMPPYWGLGFHLCRWGYSSTAITRQVVENMTRAHFPLDVQWNDLDYMDS
RRDFTFNKDGFRDFPAMVQELHQGGRRYMMIVDPAISSSGPAGSYRPYDE
GLRRGVFITNETGQPLIGKVWPGSTAFPDFTNPTALAWWEDMVAEFHDQV
PFDGMWIDMNEPSNFIRGSEDGCPNNELENPPYVPGVVGGTLQAATICAS
SHQFLSTHYNLHNLYGLTEAIASHRALVKARGTRPFVISRSTFAGHGRYA
GHWTGDVWSSWEQLASSVPEILQFNLLGVPLVGADVCGFLGNTSEELCVR
WTQLGAFYPFMRNHNSLLSLPQEPYSFSEPAQQAMRKALTLRYALLPHLY
TLFHQAHVAGETVARPLFLEFPKDSSTWTVDHQLLWGEALLITPVLQAGK
AEVTGYFPLGTWYDLQTVPVEALGSLPPPPAAPREPAIHSEGQWVTLPAP
LDTINVHLRAGYIIPLQGPGLTTTESRQQPMALAVALTKGGEARGELFWD
DGESLEVLERGAYTQVIFLARNNTIVNELVRVTSEGAGLQLQKVTVLGVA
TAPQQVLSNGVPVSNFTYSPDTKVLDICVSLLMGEQFLVSWC(SEQ ID
NO: 696)

69326

EIVMTQSPATLSVSPGERATLSCRASQSVSSNFAWYQQKPGQAPRLLIYS
ASSRATGIPVRFSGSGSGTEFTLTISSLQSEDFAVYYCQQYNIWPRTFGQ
GTKVEIKGGGGSGGGGSGGGGSEVQLVESGGGLVQPGGSLRLSCAVSGFI
FSSYEMNWVRQAPGKGLEWVSYISSSGSTIFYADSVKGRFTISRDNAKNS
LYLQMNSLRAEDTAVYYCVSGVVLFDVWGQGTMVTVSSGGGGSGGGGSAH
PGRPRAVPTQCDVPPNSRFDCAPDKAITQEQCEARGCCYIPAKQGLQGAQ
MGQPWCFFPPSYPSYKLENLSSSEMGYTATLTRTTPTFFPKDILTLRLDV
MMETENRLHFTIKDPANRRYEVPLETPHVHSRAPSPLYSVEFSEEPFGVI
VRRQLDGRVLLNTTVAPLFFADQFLQLSTSLPSQYITGLAEHLSPLMLST
SWTRITLWNRDLAPTPGANLYGSHPFYLALEDGGSAHGVFLLNSNAMDVV
LQPSPALSWRSTGGILDVYIFLGPEPKSVVQQYLDVVGYPFMPPYWGLGF
HLCRWGYSSTAITRQVVENMTRAHFPLDVQWNDLDYMDSRRDFTFNKDGF
RDFPAMVQELHQGGRRYMMIVDPAISSSGPAGSYRPYDEGLRRGVFITNE
TGQPLIGKVWPGSTAFPDFTNPTALAWWEDMVAEFHDQVPFDGMWIDMNE
PSNFIRGSEDGCPNNELENPPYVPGVVGGTLQAATICASSHQFLSTHYNL
HNLYGLTEAIASHRALVKARGTRPFVISRSTFAGHGRYAGHWTGDVWSSW
EQLASSVPEILQFNLLGVPLVGADVCGFLGNTSEELCVRWTQLGAFYPFM
RNHNSLLSLPQEPYSFSEPAQQAMRKALTLRYALLPHLYTLFHQAHVAGE
TVARPLFLEFPKDSSTWTVDHQLLWGEALLITPVLQAGKAEVTGYFPLGT
WYDLQTVPVEALGSLPPPPAAPREPAIHSEGQWVTLPAPLDTINVHLRAG
YIIPLQGPGLTTTESRQQPMALAVALTKGGEARGELFWDDGESLEVLERG
AYTQVIFLARNNTIVNELVRVTSEGAGLQLQKVTVLGVATAPQQVLSNGV
PVSNFTYSPDTKVLDICVSLLMGEQFLVSWC(SEQ ID NO: 697)

69329

DIQMTQSPSSVSASVGDRVTITCRASQGISSWLAWYQQKPGKAPKLLIYA
ASSLQSGVPSRFSGSGSGTDFTLTISSLQPEDFATYYCQKANSFPYTFGQ
GTKLEIKGGGGSGGGGSGGGGSEVQLVESGGGLVQPGGSLRLSCAASGFT
FSNYWMTWVRQAPGKGLEWVANIKEDGSEKDYVDSVKGRFTISRDNAKNS
LYLQMNSLRGEDTAVYYCARDGEQLVDYYYYYVMDVWGQGTTVTVSSGGG
GSGGGGSAHPGRPRAVPTQCDVPPNSRFDCAPDKAITQEQCEARGCCYIP
AKQGLQGAQMGQPWCFFPPSYPSYKLENLSSSEMGYTATLTRTTPTFFPK
DILTLRLDVMMETENRLHFTIKDPANRRYEVPLETPHVHSRAPSPLYSVE
FSEEPFGVIVRRQLDGRVLLNTTVAPLFFADQFLQLSTSLPSQYITGLAE
HLSPLMLSTSWTRITLWNRDLAPTPGANLYGSHPFYLALEDGGSAHGVFL
LNSNAMDVVLQPSPALSWRSTGGILDVYIFLGPEPKSVVQQYLDVVGYPF
MPPYWGLGFHLCRWGYSSTAITRQVVENMTRAHFPLDVQWNDLDYMDSRR
DFTFNKDGFRDFPAMVQELHQGGRRYMMIVDPAISSSGPAGSYRPYDEGL
RRGVFITNETGQPLIGKVWPGSTAFPDFTNPTALAWWEDMVAEFHDQVPF
DGMWIDMNEPSNFIRGSEDGCPNNELENPPYVPGVVGGTLQAATICASSH
QFLSTHYNLHNLYGLTEAIASHRALVKARGTRPFVISRSTFAGHGRYAGH
WTGDVWSSWEQLASSVPEILQFNLLGVPLVGADVCGFLGNTSEELCVRWT
QLGAFYPFMRNHNSLLSLPQEPYSFSEPAQQAMRKALTLRYALLPHLYTL
FHQAHVAGETVARPLFLEFPKDSSTWTVDHQLLWGEALLITPVLQAGKAE
VTGYFPLGTWYDLQTVPVEALGSLPPPPAAPREPAIHSEGQWVTLPAPLD
TINVHLRAGYIIPLQGPGLTTTESRQQPMALAVALTKGGEARGELFWDDG
ESLEVLERGAYTQVIFLARNNTIVNELVRVTSEGAGLQLQKVTVLGVATA
PQQVLSNGVPVSNFTYSPDTKVLDICVSLLMGEQFLVSWC(SEQ ID NO
: 698)

69331

DIQLTQSPSSLSASVGDRVTITCWASQGISSYLAWYQQKPGKAPKLLIYA
ASTLQSGVPSRFSGSGSGTEFTLTISSLQPEDFATYYCQQLNSYPLTFGG
GTKVEIKGGGGSGGGGSGGGGSQVQLVESGGGVVQPGRSLRLSCIASGFT
FSVYGIHWVRQAPGKGLEWMAVISHDGNIKHYADSVKGRFTISRDNSKNT
LYLQINSLRTEDTAVYYCAKDTWNSLDTFDIWGQGTMVTVSSGGGGSGGG
GSAHPGRPRAVPTQCDVPPNSRFDCAPDKAITQEQCEARGCCYIPAKQGL
QGAQMGQPWCFFPPSYPSYKLENLSSSEMGYTATLTRTTPTFFPKDILTL
RLDVMMETENRLHFTIKDPANRRYEVPLETPHVHSRAPSPLYSVEFSEEP
FGVIVRRQLDGRVLLNTTVAPLFFADQFLQLSTSLPSQYITGLAEHLSPL
MLSTSWTRITLWNRDLAPTPGANLYGSHPFYLALEDGGSAHGVFLLNSNA
MDVVLQPSPALSWRSTGGILDVYIFLGPEPKSVVQQYLDVVGYPFMPPYW
GLGFHLCRWGYSSTAITRQVVENMTRAHFPLDVQWNDLDYMDSRRDFTFN
KDGFRDFPAMVQELHQGGRRYMMIVDPAISSSGPAGSYRPYDEGLRRGVF
ITNETGQPLIGKVWPGSTAFPDFTNPTALAWWEDMVAEFHDQVPFDGMWI
DMNEPSNFIRGSEDGCPNNELENPPYVPGVVGGTLQAATICASSHQFLST
HYNLHNLYGLTEAIASHRALVKARGTRPFVISRSTFAGHGRYAGHWTGDV
WSSWEQLASSVPEILQFNLLGVPLVGADVCGFLGNTSEELCVRWTQLGAF
YPFMRNHNSLLSLPQEPYSFSEPAQQAMRKALTLRYALLPHLYTLFHQAH
VAGETVARPLFLEFPKDSSTWTVDHQLLWGEALLITPVLQAGKAEVTGYF
PLGTWYDLQTVPVEALGSLPPPPAAPREPAIHSEGQWVTLPAPLDTINVH
LRAGYIIPLQGPGLTTTESRQQPMALAVALTKGGEARGELFWDDGESLEV
LERGAYTQVIFLARNNTIVNELVRVTSEGAGLQLQKVTVLGVATAPQQVL
SNGVPVSNFTYSPDTKVLDICVSLLMGEQFLVSWC(SEQ ID NO: 699
)

69332

AIQMTQSPSSLSASVGDRVTITCRASQGIRNDLGWYQQKPGKAPKLLIYA
ASTLQSGVPSRFSGSGSGTDFTLTISSLQPEDFATYYCLQDYNYPFTFGP
GTKVDIKGGGGSGGGGSGGGGSQVTLRESGPALVKPSQTLTLTCTFSGFS
LNTYGMFVSWIRQPPGKALEWLAHIHWDDDKYYSTSLKTRLTISKDTSKN
QVVLTMTNMDPVDTATYYCARGHNNLNYIIHWGQGTLVTVSSGGGGSGGG
GSAHPGRPRAVPTQCDVPPNSRFDCAPDKAITQEQCEARGCCYIPAKQGL
QGAQMGQPWCFFPPSYPSYKLENLSSSEMGYTATLTRTTPTFFPKDILTL
RLDVMMETENRLHFTIKDPANRRYEVPLETPHVHSRAPSPLYSVEFSEEP
FGVIVRRQLDGRVLLNTTVAPLFFADQFLQLSTSLPSQYITGLAEHLSPL
MLSTSWTRITLWNRDLAPTPGANLYGSHPFYLALEDGGSAHGVFLLNSNA
MDVVLQPSPALSWRSTGGILDVYIFLGPEPKSVVQQYLDVVGYPFMPPYW
GLGFHLCRWGYSSTAITRQVVENMTRAHFPLDVQWNDLDYMDSRRDFTFN
KDGFRDFPAMVQELHQGGRRYMMIVDPAISSSGPAGSYRPYDEGLRRGVF
ITNETGQPLIGKVWPGSTAFPDFTNPTALAWWEDMVAEFHDQVPFDGMWI
DMNEPSNFIRGSEDGCPNNELENPPYVPGVVGGTLQAATICASSHQFLST
HYNLHNLYGLTEAIASHRALVKARGTRPFVISRSTFAGHGRYAGHWTGDV
WSSWEQLASSVPEILQFNLLGVPLVGADVCGFLGNTSEELCVRWTQLGAF
YPFMRNHNSLLSLPQEPYSFSEPAQQAMRKALTLRYALLPHLYTLFHQAH
VAGETVARPLFLEFPKDSSTWTVDHQLLWGEALLITPVLQAGKAEVTGYF
PLGTWYDLQTVPVEALGSLPPPPAAPREPAIHSEGQWVTLPAPLDTINVH
LRAGYIIPLQGPGLTTTESRQQPMALAVALTKGGEARGELFWDDGESLEV
LERGAYTQVIFLARNNTIVNELVRVTSEGAGLQLQKVTVLGVATAPQQVL
SNGVPVSNFTYSPDTKVLDICVSLLMGEQFLVSWC(SEQ ID NO: 700
)

69340

EIVLTQSPATLSLSPGERATLSCRASQSVSSYLAWYQQKPGQAPRLLIHD
VSNRATGIPARFSGSGSGTDFTLTISSLEPEDFVVYYCQQRSDWPITFGQ
GTRLEIKGGGGSGGGGSGGGGSEVQLVESGGGLVQPGRSLRLSCAASGFT
FDDKAMHWVRQVPGKGLEWISGISWNSGTIGYADSVKGRFIISRDNAKNS
LYLQMNSLRAEDTALYYCAKDGDTSGWYWYGLDVWGQGTTVTVSSGGGGS
GGGGSAHPGRPRAVPTQCDVPPNSRFDCAPDKAITQEQCEARGCCYIPAK
QGLQGAQMGQPWCFFPPSYPSYKLENLSSSEMGYTATLTRTTPTFFPKDI
LTLRLDVMMETENRLHFTIKDPANRRYEVPLETPHVHSRAPSPLYSVEFS
EEPFGVIVRRQLDGRVLLNTTVAPLFFADQFLQLSTSLPSQYITGLAEHL
SPLMLSTSWTRITLWNRDLAPTPGANLYGSHPFYLALEDGGSAHGVFLLN
SNAMDVVLQPSPALSWRSTGGILDVYIFLGPEPKSVVQQYLDVVGYPFMP
PYWGLGFHLCRWGYSSTAITRQVVENMTRAHFPLDVQWNDLDYMDSRRDF
TFNKDGFRDFPAMVQELHQGGRRYMMIVDPAISSSGPAGSYRPYDEGLRR
GVFITNETGQPLIGKVWPGSTAFPDFTNPTALAWWEDMVAEFHDQVPFDG
MWIDMNEPSNFIRGSEDGCPNNELENPPYVPGVVGGTLQAATICASSHQF
LSTHYNLHNLYGLTEAIASHRALVKARGTRPFVISRSTFAGHGRYAGHWT
GDVWSSWEQLASSVPEILQFNLLGVPLVGADVCGFLGNTSEELCVRWTQL
GAFYPFMRNHNSLLSLPQEPYSFSEPAQQAMRKALTLRYALLPHLYTLFH
QAHVAGETVARPLFLEFPKDSSTWTVDHQLLWGEALLITPVLQAGKAEVT
GYFPLGTWYDLQTVPVEALGSLPPPPAAPREPAIHSEGQWVTLPAPLDTI
NVHLRAGYIIPLQGPGLTTTESRQQPMALAVALTKGGEARGELFWDDGES
LEVLERGAYTQVIFLARNNTIVNELVRVTSEGAGLQLQKVTVLGVATAPQ
QVLSNGVPVSNFTYSPDTKVLDICVSLLMGEQFLVSWC(SEQ ID NO:
701)

69348

DIQMTQSPSSLSASVGDRVTITCRASQSIRNVLGWFQQKPGKAPQRLIYA
ASSLQSGVPSRFSGSGSGTEFTLTISSLQPEDFATYYCLQHNFYPLTFGG
GTKVEIKGGGGSGGGGSGGGGSQVQLVESGGGVVQPGRSLRLSCAASGFT
FTTYGMHWVRQAPGKGLEWVAVIWYDGSNKYYGDSVKGRFTISRDNSKNT
LYLQMNSLRVDDTAVYYCTRTHGYTRSSDGFDYWGQGTLVTVSSGGGGSG
GGGSAHPGRPRAVPTQCDVPPNSRFDCAPDKAITQEQCEARGCCYIPAKQ
GLQGAQMGQPWCFFPPSYPSYKLENLSSSEMGYTATLTRTTPTFFPKDIL
TLRLDVMMETENRLHFTIKDPANRRYEVPLETPHVHSRAPSPLYSVEFSE
EPFGVIVRRQLDGRVLLNTTVAPLFFADQFLQLSTSLPSQYITGLAEHLS
PLMLSTSWTRITLWNRDLAPTPGANLYGSHPFYLALEDGGSAHGVFLLNS
NAMDVVLQPSPALSWRSTGGILDVYIFLGPEPKSVVQQYLDVVGYPFMPP
YWGLGFHLCRWGYSSTAITRQVVENMTRAHFPLDVQWNDLDYMDSRRDFT
FNKDGFRDFPAMVQELHQGGRRYMMIVDPAISSSGPAGSYRPYDEGLRRG
VFITNETGQPLIGKVWPGSTAFPDFTNPTALAWWEDMVAEFHDQVPFDGM
WIDMNEPSNFIRGSEDGCPNNELENPPYVPGVVGGTLQAATICASSHQFL
STHYNLHNLYGLTEAIASHRALVKARGTRPFVISRSTFAGHGRYAGHWTG
DVWSSWEQLASSVPEILQFNLLGVPLVGADVCGFLGNTSEELCVRWTQLG
AFYPFMRNHNSLLSLPQEPYSFSEPAQQAMRKALTLRYALLPHLYTLFHQ
AHVAGETVARPLFLEFPKDSSTWTVDHQLLWGEALLITPVLQAGKAEVTG
YFPLGTWYDLQTVPVEALGSLPPPPAAPREPAIHSEGQWVTLPAPLDTIN
VHLRAGYIIPLQGPGLTTTESRQQPMALAVALTKGGEARGELFWDDGESL
EVLERGAYTQVIFLARNNTIVNELVRVTSEGAGLQLQKVTVLGVATAPQQ
VLSNGVPVSNFTYSPDTKVLDICVSLLMGEQFLVSWC(SEQ ID NO: 7
02);

MHRPRRRGTRPPPLALLAALLLAARGADADIQMTQSPSSVSASVGDRVTI
TCRASQGIASWLAWYQQKPGKAPELLIYAASSLQGGVPSRFSGSGSGTDF
TLTISSLQPEDFAIYYCQQANYFPWTFGQGTKVEIKGGGGSGGGGSGGGG
SQITLKESGPTLVKPTQTLTLTCTFSGFSLSTSGVGVVWIRQPPGKALEW
LALIYWNDHKRYSPSLGSRLTITKDTSKNQVVLTMTNMDPVDTATYYCAH
YSGSYSYYYYGLDVWGQGTTVTVSSGGGGSGGGGSAHPGRPRAVPTQCDV
PPNSRFDCAPDKAITQEQCEARGCCYIPAKQGLQGAQMGQPWCFFPPSYP
SYKLENLSSSEMGYTATLTRTTPTFFPKDILTLRLDVMMETENRLHFTIK
DPANRRYEVPLETPHVHSRAPSPLYSVEFSEEPFGVIVRRQLDGRVLLNT
TVAPLFFADQFLQLSTSLPSQYITGLAEHLSPLMLSTSWTRITLWNRDLA
PTPGANLYGSHPFYLALEDGGSAHGVFLLNSNAMDVVLQPSPALSWRSTG
GILDVYIFLGPEPKSVVQQYLDVVGYPFMPPYWGLGFHLCRWGYSSTAIT
RQVVENMTRAHFPLDVQWNDLDYMDSRRDFTFNKDGFRDFPAMVQELHQG
GRRYMMIVDPAISSSGPAGSYRPYDEGLRRGVFITNETGQPLIGKVWPGS
TAFPDFTNPTALAWWEDMVAEFHDQVPFDGMWIDMNEPSNFIRGSEDGCP
NNELENPPYVPGVVGGTLQAATICASSHQFLSTHYNLHNLYGLTEAIASH
RALVKARGTRPFVISRSTFAGHGRYAGHWTGDVWSSWEQLASSVPEILQF
NLLGVPLVGADVCGFLGNTSEELCVRWTQLGAFYPFMRNHNSLLSLPQEP
YSFSEPAQQAMRKALTLRYALLPHLYTLFHQAHVAGETVARPLFLEFPKD
SSTWTVDHQLLWGEALLITPVLQAGKAEVTGYFPLGTWYDLQTVPVEALG
SLPPPPAAPREPAIHSEGQWVTLPAPLDTINVHLRAGYIIPLQGPGLTTT
ESRQQPMALAVALTKGGEARGELFWDDGESLEVLERGAYTQVIFLARNNT
IVNELVRVTSEGAGLQLQKVTVLGVATAPQQVLSNGVPVSNFTYSPDTKV
LDICVSLLMGEQFLVSWC(SEQ ID NO: 703;

optionally lacking the N-terminal MHRPRRRGTRPPPLALLAALLLAARGADA (SEQ ID NO: 709) sequence);

MHRPRRRGTRPPPLALLAALLLAARGADADIQMTQSPSSLSASVGDRVTI
TCRASQSISSYLNWYQQKPGKAPKLLIYAASSLQSGVPSRFSGSGSGTDF
TLTISSLQPEDFATYYCQQSYSTPPITFGQGTRLEIKGGGGSGGGGSGGG
GSQVQLVESGGGVVQPGRSLRLSCAASGFPFSNYVMYWVRQAPGKGLEWV
ALIFFDGKKNYHADSVKGRFTITRDNSKNMLYLQMNSLRPEDAAVYYCAK
IHCPNGVCYKGYYGMDVWGQGTTVTVSSGGGGSGGGGSAHPGRPRAVPTQ
CDVPPNSRFDCAPDKAITQEQCEARGCCYIPAKQGLQGAQMGQPWCFFPP
SYPSYKLENLSSSEMGYTATLTRTTPTFFPKDILTLRLDVMMETENRLHF
TIKDPANRRYEVPLETPHVHSRAPSPLYSVEFSEEPFGVIVRRQLDGRVL
LNTTVAPLFFADQFLQLSTSLPSQYITGLAEHLSPLMLSTSWTRITLWNR
DLAPTPGANLYGSHPFYLALEDGGSAHGVFLLNSNAMDVVLQPSPALSWR
STGGILDVYIFLGPEPKSVVQQYLDVVGYPFMPPYWGLGFHLCRWGYSST
AITRQVVENMTRAHFPLDVQWNDLDYMDSRRDFTFNKDGFRDFPAMVQEL
HQGGRRYMMIVDPAISSSGPAGSYRPYDEGLRRGVFITNETGQPLIGKVW
PGSTAFPDFTNPTALAWWEDMVAEFHDQVPFDGMWIDMNEPSNFIRGSED
GCPNNELENPPYVPGVVGGTLQAATICASSHQFLSTHYNLHNLYGLTEAI
ASHRALVKARGTRPFVISRSTFAGHGRYAGHWTGDVWSSWEQLASSVPEI
LQFNLLGVPLVGADVCGFLGNTSEELCVRWTQLGAFYPFMRNHNSLLSLP
QEPYSFSEPAQQAMRKALTLRYALLPHLYTLFHQAHVAGETVARPLFLEF
PKDSSTWTVDHQLLWGEALLITPVLQAGKAEVTGYFPLGTWYDLQTVPVE
ALGSLPPPPAAPREPAIHSEGQWVTLPAPLDTINVHLRAGYIIPLQGPGL
TTTESRQQPMALAVALTKGGEARGELFWDDGESLEVLERGAYTQVIFLAR
NNTIVNELVRVTSEGAGLQLQKVTVLGVATAPQQVLSNGVPVSNFTYSPD
TKVLDICVSLLMGEQFLVSWC(SEQ ID NO: 704;

optionally lacking the N-terminal MHRPRRRGTRPPPLALLAALLLAARGADA (SEQ ID NO: 709) sequence);

MHRPRRRGTRPPPLALLAALLLAARGADADIQMTQSPSSLSASVGDRVTI
TCRASQSISSYLNWYQQKPGKAPKLLIYAASSLQSGVPSRFSGSGSGTDF
TLTISSLQPEDFATYYCQQSYSTPPITFGQGTRLEIKGGGGSGGGGSGGG
GSEVQLVESGGGLVQPGGSLRLSCAASGFTFNIFEMNWVRQAPGKGLEWI
SYISSRGTTTYYADSVRGRFTISRDNAKNSLYLQMNSLRAEDTAVYYCAR
DYEATIPFDFWGQGTLVTVSSGGGGSGGGGSAHPGRPRAVPTQCDVPPNS
RFDCAPDKAITQEQCEARGCCYIPAKQGLQGAQMGQPWCFFPPSYPSYKL
ENLSSSEMGYTATLTRTTPTFFPKDILTLRLDVMMETENRLHFTIKDPAN
RRYEVPLETPHVHSRAPSPLYSVEFSEEPFGVIVRRQLDGRVLLNTTVAP
LFFADQFLQLSTSLPSQYITGLAEHLSPLMLSTSWTRITLWNRDLAPTPG
ANLYGSHPFYLALEDGGSAHGVFLLNSNAMDVVLQPSPALSWRSTGGILD
VYIFLGPEPKSVVQQYLDVVGYPFMPPYWGLGFHLCRWGYSSTAITRQVV
ENMTRAHFPLDVQWNDLDYMDSRRDFTFNKDGFRDFPAMVQELHQGGRRY
MMIVDPAISSSGPAGSYRPYDEGLRRGVFITNETGQPLIGKVWPGSTAFP
DFTNPTALAWWEDMVAEFHDQVPFDGMWIDMNEPSNFIRGSEDGCPNNEL
ENPPYVPGVVGGTLQAATICASSHQFLSTHYNLHNLYGLTEAIASHRALV
KARGTRPFVISRSTFAGHGRYAGHWTGDVWSSWEQLASSVPEILQFNLLG
VPLVGADVCGFLGNTSEELCVRWTQLGAFYPFMRNHNSLLSLPQEPYSFS
EPAQQAMRKALTLRYALLPHLYTLFHQAHVAGETVARPLFLEFPKDSSTW
TVDHQLLWGEALLITPVLQAGKAEVTGYFPLGTWYDLQTVPVEALGSLPP
PPAAPREPAIHSEGQWVTLPAPLDTINVHLRAGYIIPLQGPGLTTTESRQ
QPMALAVALTKGGEARGELFWDDGESLEVLERGAYTQVIFLARNNTIVNE
LVRVTSEGAGLQLQKVTVLGVATAPQQVLSNGVPVSNFTYSPDTKVLDIC
VSLLMGEQFLVSWC(SEQ ID NO: 705;

optionally lacking the N-terminal MHRPRRRGTRPPPLALLAALLLAARGADA (SEQ ID NO: 709) sequence);

MHRPRRRGTRPPPLALLAALLLAARGADADIQMTQSPSSLSASVGDRVTI
TCRASQSISSYLNWYQQKPGKAPKLLIYAASSLQSGVPSRFSGSGSGTDF
TLTISSLQPEDFATYYCQQSYSTPPITFGQGTRLEIKGGGGSGGGGSGGG
GSEVQLVESGGGLVQPGRSLRLSCAASGFTFDDYAMNWVRQAPGKGLEWV
SGISWSSGSMDYADSVKGRFTISRDNAKNSLYLQMNSLRTEDTALYYCAK
AREVGDYYGMDVWGQGTTVTVSSGGGGSGGGGSAHPGRPRAVPTQCDVPP
NSRFDCAPDKAITQEQCEARGCCYIPAKQGLQGAQMGQPWCFFPPSYPSY
KLENLSSSEMGYTATLTRTTPTFFPKDILTLRLDVMMETENRLHFTIKDP
ANRRYEVPLETPHVHSRAPSPLYSVEFSEEPFGVIVRRQLDGRVLLNTTV
APLFFADQFLQLSTSLPSQYITGLAEHLSPLMLSTSWTRITLWNRDLAPT
PGANLYGSHPFYLALEDGGSAHGVFLLNSNAMDVVLQPSPALSWRSTGGI
LDVYIFLGPEPKSVVQQYLDVVGYPFMPPYWGLGFHLCRWGYSSTAITRQ
VVENMTRAHFPLDVQWNDLDYMDSRRDFTFNKDGFRDFPAMVQELHQGGR
RYMMIVDPAISSSGPAGSYRPYDEGLRRGVFITNETGQPLIGKVWPGSTA
FPDFTNPTALAWWEDMVAEFHDQVPFDGMWIDMNEPSNFIRGSEDGCPNN
ELENPPYVPGVVGGTLQAATICASSHQFLSTHYNLHNLYGLTEAIASHRA
LVKARGTRPFVISRSTFAGHGRYAGHWTGDVWSSWEQLASSVPEILQFNL
LGVPLVGADVCGFLGNTSEELCVRWTQLGAFYPFMRNHNSLLSLPQEPYS
FSEPAQQAMRKALTLRYALLPHLYTLFHQAHVAGETVARPLFLEFPKDSS
TWTVDHQLLWGEALLITPVLQAGKAEVTGYFPLGTWYDLQTVPVEALGSL
PPPPAAPREPAIHSEGQWVTLPAPLDTINVHLRAGYIIPLQGPGLTTTES
RQQPMALAVALTKGGEARGELFWDDGESLEVLERGAYTQVIFLARNNTIV
NELVRVTSEGAGLQLQKVTVLGVATAPQQVLSNGVPVSNFTYSPDTKVLD
ICVSLLMGEQFLVSWC(SEQ ID NO: 706;

optionally lacking the N-terminal MHRPRRRGTRPPPLALLAALLLAARGADA (SEQ ID NO: 709) sequence)

In order to validate the anti-human TfR antibodies that were screened for binding in vitro, we performed in vivo mouse studies in Tfrc^{hum/hum} knock-in mice to evaluate blood-brain-barrier (BBB) crossing. Eleven clones that had mature hGAA protein in brain homogenate detected by western blot were selected from this first screen of 31 antibodies.

GAA fusions by hydrodynamic delivery (HDD). Three-month-old human TFRC knock-in mice were injected with DNA plasmids expressing the various anti-hTfR antibodies in the anti-hTfRscfv:2xG4S:hGAA format under the liver-specific mouse TTR promoter. Mice received 50 µg of DNA in 0.9% sterile saline diluted to 10% of the mouse’s body weight (0.1 mL/g body weight). 48 hours post-injection, tissues were dissected from mice immediately after sacrifice by CO₂ asphyxiation, snap frozen in liquid nitrogen, and stored at -80° C.

Tissue lysates were prepared by lysis in RIPA buffer with protease inhibitors (1861282, Thermo Fisher, Waltham, MA, USA). Tissue lysates were homogenized with a bead homogenizer (FastPrep5, MP Biomedicals, Santa Ana, CA, USA). Cells or tissue lysates were run on SDS-PAGE gels using the Novex system (LifeTech Thermo, XPO4200BOX, LC2675, LC3675, LC2676). Gels were transferred to low-fluorescence polyvinylidene fluoridev (PVDF) membrane (IPFL07810, LI-COR, Lincoln, NE, USA) and stained with Revert 700 Total Protein Stain (TPS; 926-11010 LI-COR, Lincoln, NE, USA), followed by blocking with Odyssey blocking buffer (927-500000, LI-COR, Lincoln, NE, USA) in Tris buffer saline with 0.1% Tween 20 and staining with antibodies against GAA (ab137068, Abcam, Cambridge, MA, USA), or anti-GAPDH (ab9484, Abcam, Cambridge, MA, USA) and the appropriate secondary (926-32213 or 925-68070, LI-COR, Lincoln, NE, USA). Blots were imaged with a LI-COR Odyssey CLx.

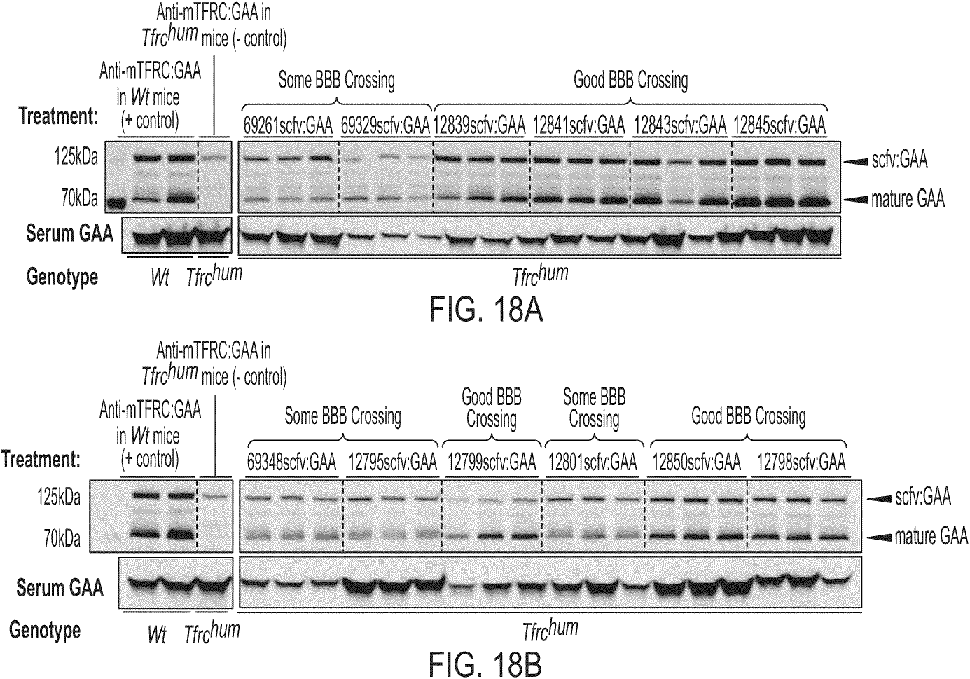

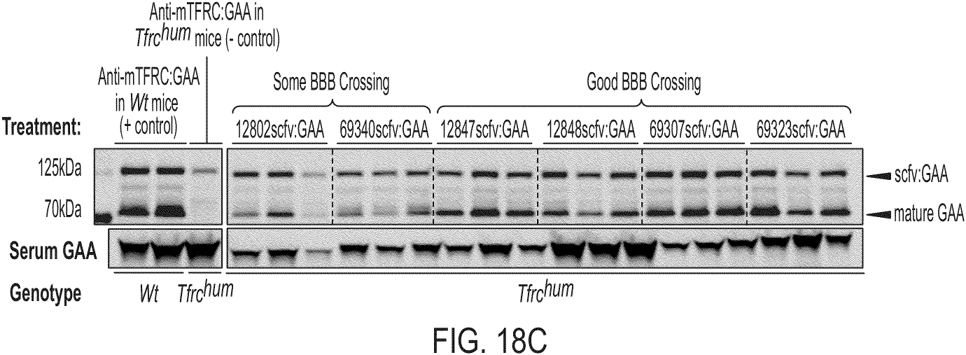

Protein band intensity was quantified in LI-COR Image Studio software. The quantification of the mature 77 kDa GAA band for each sample was determined by first normalizing to the lane’s TPS signal, then normalizing to GAA levels in the serum (loading control and liver expression control, respectively). Values were then compared to the positive control group anti-mouse TfRscfv:hGAA in Wt mice, and negative control group anti-mTfRscfv:hGAA in Tfrc^{hum/hum} mice (FIGS. 18A-18C, Table 31). The 8D3 scFv (anti-mouse TfR scFv) has the heavy chain amino acid sequence:

EVQLVESGGGLVQPGNSLTLSCVASGFTFSNYGMHWIRQAPKKGLEWIAM
IYYDSSKMNYADTVKGRFTISRDNSKNTLYLEMNSLRSEDTAMYYCAVPT
SHYVVDVWGQGVSVTVSS (SEQ ID NO: 536),

and the light chain amino acid sequence:

DIQMTQSPASLSASLEEIVTITCQASQDIGNWLAWYQQKPGKSPQLLIYG
ATSLADGVPSRFSGSRSGTQFSLKISRVQVEDIGIYYCLQAYNTPWTFGG
GTKLELK (SEQ ID NO: 537).

TABLE 31

Quantification of mature hGAA protein in brain homogenate from mice treated HDD with anti-hTfRscfv:hGAA plasmids

| Treatment group | Genotype | Mature hGAA protein in brain (normalized to positive control) |
|---|---|---|
| anti-mTfRscfv:hGAA (positive control) | Wt | 1.00±0.43^{∗} |
| anti-mTfRscfv:hGAA (negative control) | Tfrc^{hum/hum} | 0.02±0.03 |
| 69261scfv:hGAA | Tfrc^{hum/hum} | 0.67±0.50 |
| 69307scfv:hGAA | Tfrc^{hum/hum} | 1.08±0.19 |
| 69323scfv:hGAA | Tfrc^{hum/hum} | 0.91±0.46 |
| 69329scfv:hGAA | Tfrc^{hum/hum} | 0.65±0.13 |
| 69340scfv:hGAA | Tfrc^{hum/hum} | 0.55±0.58 |
| 69348scfv:hGAA | Tfrc^{hum/hum} | 0.50±0.05 |
| 12795scfv:hGAA | Tfrc^{hum/hum} | 0.27±0.20 |
| 12798scfv:hGAA | Tfrc^{hum/hum} | 0.72±0.42 |
| 12799scfv:hGAA | Tfrc^{hum/hum} | 1.05±0.51^{∗} |
| 12801scfv:hGAA | Tfrc^{hum/hum} | 0.49±0.18 |
| 12802scfv:hGAA | Tfrc^{hum/hum} | 0.29±0.27 |
| 12839scfv:hGAA | Tfrc^{hum/hum} | 1.29±0.27^{∗∗} |
| 12841scfv:hGAA | Tfrc^{hum/hum} | 1.72±0.06^{∗∗∗} |
| 12843scfv:hGAA | Tfrc^{hum/hum} | 1.79±0.85^{∗∗∗} |
| 12845scfv:hGAA | Tfrc^{hum/hum} | 3.08±0.92^{∗∗∗} |
| 12847scfv:hGAA | Tfrc^{hum/hum} | 1.24±0.30 |
| 12848scfv:hGAA | Tfrc^{hum/hum} | 0.59±0.16 |
| 12850scfv:hGAA | Tfrc^{hum/hum} | 0.47±0.05 |

Data were quantified from western blot as arbitrary units (FIGS. 18A-18C). All values are mean ± SD, n=3-6 per group. One Way ANOVA vs. negative control anti-mTfRscfv:hGAA in Tfrc^{hum/hum} mice; ^{∗}p<0.05; ^{∗∗}p<0.005; ^{∗∗∗}p<0.0001.

Capillary depletion of brain samples following HDD of anti-hTfRscfv:hGAA plasmids. Selected anti-hTfRscfv:hGAA from Table 31 were tested in a secondary screen in Tfrc^{hum} mice to determine whether hGAA was present in the brain parenchyma, and not trapped in the BBB endothelial cells. We selected four scFvs (12799, 12839, 12843, and 12847) from this screen based on mature hGAA in the parenchyma fraction on western blot, as well as high affinity to cynomolgus TfR.

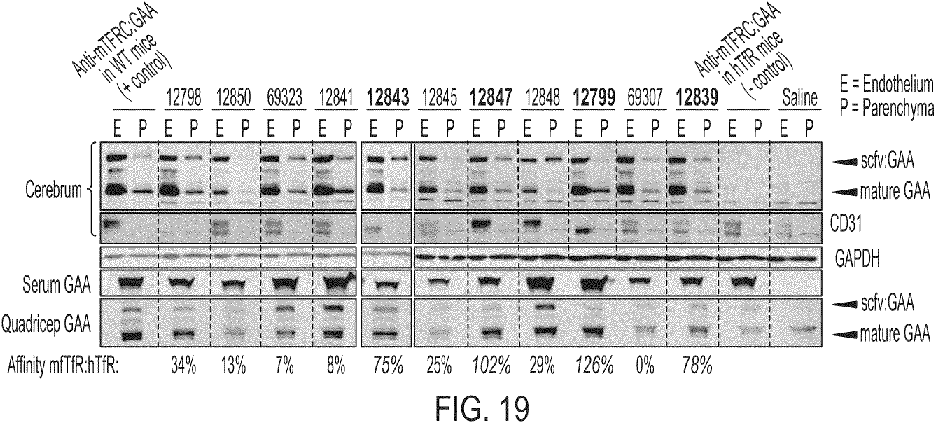

Three-month-old animals were treated HDD as detailed above. 48 hours post-injection, mice were perfused with 30 mL 0.9% saline immediately after sacrifice by CO₂ asphyxiation. A 2 mm coronal slice of cerebrum was taken between bregma and -2 mm bregma and placed in 700 µL physiological buffer (10 mM HEPES, 4 mM KCI, 2.8 mM CaCl₂, 1 mM MgSO₄, 1 mM NaH₂PO₄, 10 mM D-glucose in 0.9% saline pH 7.4) on ice. Brain slices were gently homogenized on ice with a glass dounce homogenizer. An equivalent volume of 26% dextran (MW 70,000 Da) in physiological buffer was added (final 13% dextran) and homogenized 10 more strokes. Parenchyma (supernatant) and endothelial (pellet) fractions were separated by centrifugation at 5,400 g for 15 min at 4° C. Anti-hGAA western blot was performed on fractions as detailed above (FIG. 19, Table 32). Blots were also probed with anti-CD31 endothelial marker (Abcam ab182982).

TABLE 32

Quantification of mature hGAA protein in brain parenchyma fractions and BBB endothelial fractions of mice treated HDD with anti-hTfRscfv:hGAA plasmids

| Treatment group | Genotype | Mature hGAA protein in brain parenchyma (normalized to positive control) | Mature hGAA protein in brain endothelium (normalized to positive control) | Affinity to mfTfR (% of hTfR binding) |
|---|---|---|---|---|
| anti-mTfRscfv:hGAA (positive control) | Wt | 1.00 | 5.82 | ND |
| anti-mTfRscfv:hGAA (negative control) | Tfrc^{hum/hum} | 0.00 | 0.01 | ND |
| 69307scfv:hGAA | Tfrc^{hum/hum} | 1.24 | 10.73 | 0% |
| 69323scfv:hGAA | Tfrc^{hum/hum} | 0.62 | 4.18 | 7% |
| 12798scfv:hGAA | Tfrc^{hum/hum} | 0.91 | 8.37 | 34% |
| 12799scfv:hGAA | Tfrc^{hum/hum} | 0.44 | 3.99 | 126% |
| 12839scfv:hGAA | Tfrc^{hum/hum} | 0.05 | 0.84 | 78% |
| 12841scfv:hGAA | Tfrc^{hum/hum} | 0.78 | 4.23 | 8% |
| 12843scfv:hGAA | Tfrc^{hum/hum} | 1.13 | 12.99 | 75% |
| 12845scfv:hGAA | Tfrc^{hum/hum} | 2.04 | 13.06 | 25% |
| 12847scfv:hGAA | Tfrc^{hum/hum} | 0.60 | 4.96 | 102% |
| 12848scfv:hGAA | Tfrc^{hum/hum} | 0.17 | 1.24 | 29% |
| 12850scfv:hGAA | Tfrc^{hum/hum} | 0.22 | 2.25 | 13% |

hGAA protein was quantified from western blot as arbitrary units (FIG. 19). n=1 per group. Affinity to cynomolgus macaque TfR Luminex data, calculated as percent of binding to hTfR: (mfTfR binding ÷ hTfR binding) × 100.

TABLE 33

Quantification of hGAA protein in quadricep of mice treated HDD with anti-hTfRscfv:hGAA plasmids

| Treatment group | Genotype | hGAA protein in quadricep (normalized to positive control) |
|---|---|---|
| Saline (vehicle) | Tfrc^{hum/hum} | 0.38±0.25 |
| anti-mTfRscfv:hGAA (positive control) | Wt | 1.07±0.27 |
| anti-mTfRscfv:hGAA (negative control) | Tfrc^{hum/hum} | 0.56±0.17 |
| 69307scfv:hGAA | Tfrc^{hum/hum} | 0.58±0.18 |
| 69323scfv:hGAA | Tfrc^{hum/hum} | 1.10±0.19 |
| 12798scfv:hGAA | Tfrc^{hum/hum} | 1.33±0.56 |
| 12799scfv:hGAA | Tfrc^{hum/hum} | 0.67±0.18 |
| 12839scfv:hGAA | Tfrc^{hum/hum} | 1.80±0.18 |
| 12841scfv:hGAA | Tfrc^{hum/hum} | 1.15±0.12 |
| 12843scfv:hGAA | Tfrc^{hum/hum} | 1.78±0.43 |
| 12845scfv:hGAA | Tfrc^{hum/hum} | 1.70±1.33 |
| 12847scfv:hGAA | Tfrc^{hum/hum} | 7.74±9.42 |
| 12848scfv:hGAA | Tfrc^{hum/hum} | 0.82±0.18 |
| 12850scfv:hGAA | Tfrc^{hum/hum} | 0.76±0.34 |

Data were quantified from western blot as arbitrary units (FIG. 19). All values are mean ± SD, n=2-4 per group.

Capillary depletion of mouse brain samples following liver-depot AAV8 anti-hTfRscfv:hGAA treatment. To confirm our HDD screen findings in a more long-term treatment model, we treated Tfrc^{hum} mice with selected anti-hTfRscfv:GAA delivered as episomal liver depot AAV8 anti-hTfRscfv:GAA under the TTR promoter. We found that all 4 anti-hTfRscfv:GAA delivered mature hGAA to the brain parenchyma when delivered as AAV8.

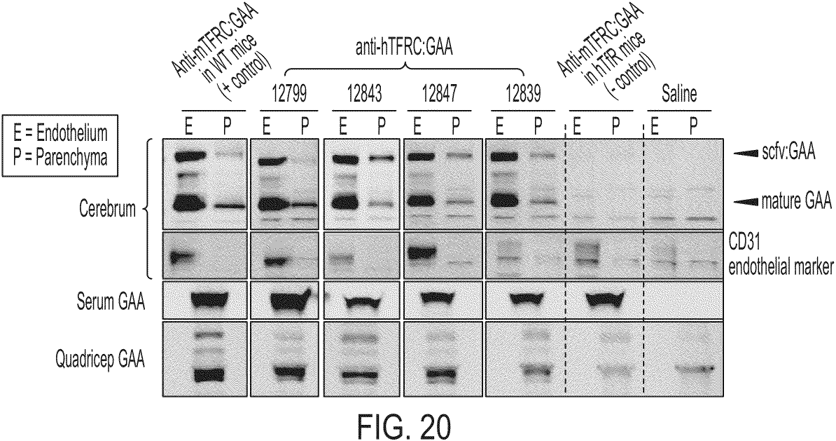

AAVproduction and in vivo transduction. Recombinant AAV8 (AAV2/8) was produced in HEK293 cells. Cells were transfected with three plasmids encoding adenovirus helper genes, AAV8 rep and cap genes, and recombinant AAV genomes containing transgenes flanked by AAV2 inverted terminal repeats (ITRs). On day 5, cells and medium were collected, centrifuged, and processed for AAV purification. Cell pellets were lysed by freeze-thaw and cleared by centrifugation. Processed cell lysates and medium were overlaid onto iodixanol gradients columns and centrifuged in an ultracentrifuge. Virus fractions were removed from the interface between the 40% and 60% iodixanol solutions and exchanged into 1xPBS with desalting columns. AAV vg were quantified by ddPCR. AAVs were diluted in PBS + 0.001% F-68 Pluronic immediately prior to injection. Three-month-old Tfrc^{hum} mice were dosed with 3e12 vg/kg body weight in a volume of ~100 µL. Mice were sacrificed 4 weeks post injection and capillary depletion and western blotting were performed as described above (FIG. 20, Table 34).

TABLE 34

Quantification of mature hGAA protein in brain parenchyma fractions and BBB endothelial fractions of mice treated with liver-depot AAV8 anti-hTfRscfv:hGAA

| Treatment group | Genotype | Mature hGAA protein in brain parenchyma (normalized to positive control) | Mature hGAA protein in brain endothelium (normalized to positive control) |
|---|---|---|---|
| anti-mTfRscfv:hGAA (positive control) | Wt | 1.00 | 1.00 |
| anti-mTfRscfv:hGAA (negative control) | Tfrc^{hum/hum} | 0.02 | 0.01 |
| 12799scfv:hGAA | Tfrc^{hum/hum} | 0.94 | 0.94 |
| 12839scfv:hGAA | Tfrc^{hum/hum} | 0.49 | 0.62 |
| 12843scfv:hGAA | Tfrc^{hum/hum} | 0.61 | 0.63 |
| 12847scfv:hGAA | Tfrc^{hum/hum} | 1.90 | 1.33 |

Data were quantified from western blot as arbitrary units (FIG. 20). n=1 per group.

Rescue of glycogen storage phenotype in Gaa^{-/-}/Tfrc^{hum} mice with AAV8 episomal liver depot anti-hTfRscfv:GAA. We tested four of the anti-hTfRscfv:GAA from the above experiment in Pompe disease model mice to determine whether hTfRscfv:GAA rescued the glycogen storage phenotype. We found that all four (12839, 12843, 12847, 12799) normalized glycogen to Wt levels.

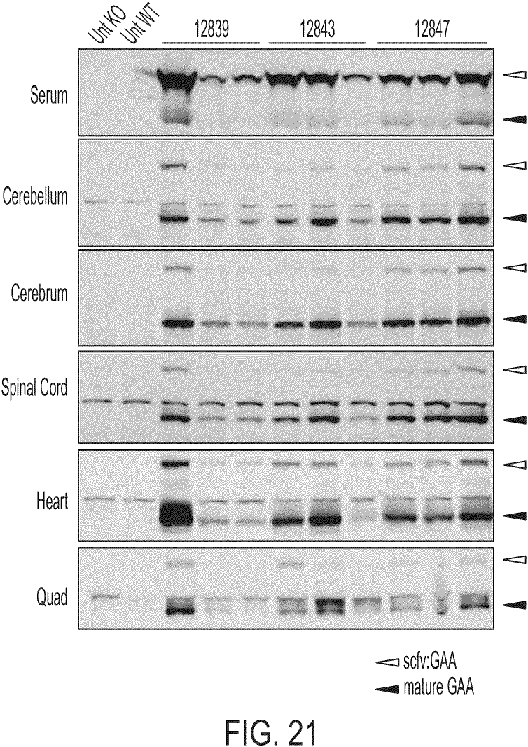

AAV production and in vivo transduction were performed as above. Three-month-old Gaa^{-/-}/Tfrc^{hum} mice were dosed with 2e12 vg/kg AAV8. Tissues were harvested 4 weeks post-injection and flash-frozen as above. hGAA Western blot was performed as above (FIG. 21, Table 35).

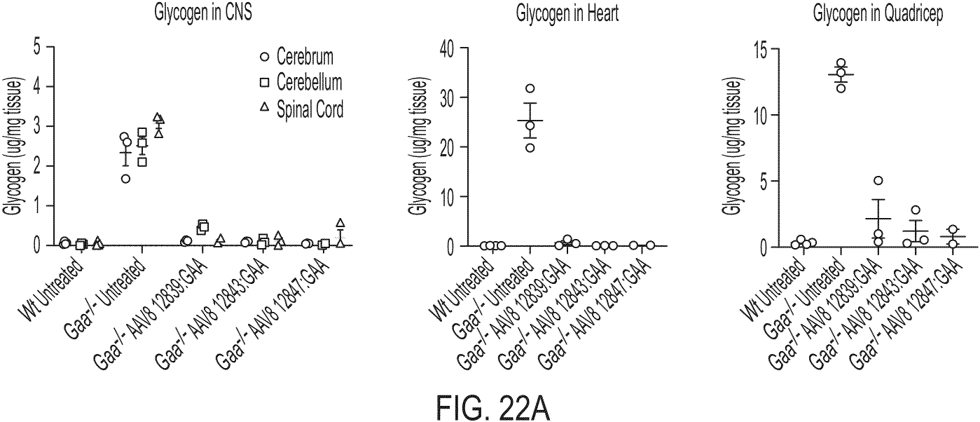

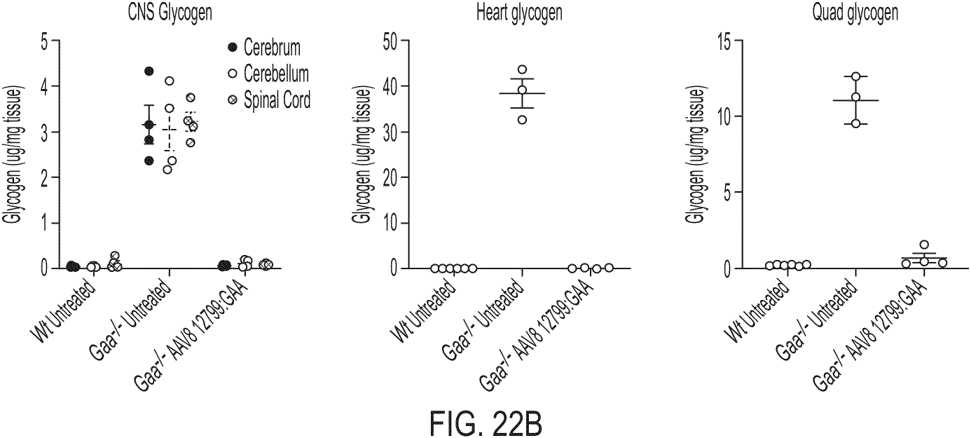

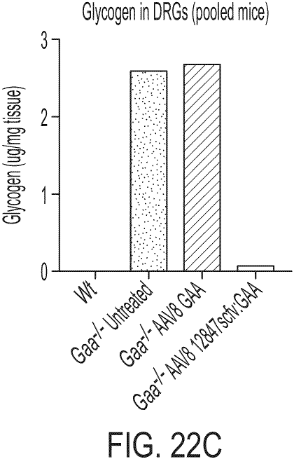

Glycogen quantification (Table 36, FIGS. 22A-22C). Tissues were dissected from mice immediately after sacrifice by CO₂ asphyxiation, snap frozen in liquid nitrogen, and stored at -80° C. Tissues were lysed on a benchtop homogenizer with stainless steel beads in distilled water for glycogen measurements or RIPA buffer for protein analyses. Glycogen analysis lysates were boiled and centrifuged to clear debris. Glycogen measurements were performed fluorometrically with a commercial kit according to manufacturer’s instructions (K646, BioVision, Milpitas, CA, USA). All groups had normal iron homeostasis at 4 weeks post-injection (serum iron, TIBC, hepcidin, tissue iron, tissue transferrin).

TABLE 35

Quantification of hGAA protein in tissues of Gaa^{-/-}/Tfrc^{hum} mice treated with liver-depot AAV8 anti-hTfRscfv:hGAA

| Treatment group | n | Serum ^{∗} | Liver ^{∗} | Cerebrum ^{∗∗} | Cerebellum ^{∗∗} | Spinal Cord ^{∗∗} | Heart ^{∗∗} | Quadricep ^{∗∗} |
|---|---|---|---|---|---|---|---|---|
| Gaa^{-/-} Untreated | 1 | 0.00 | 0.02 | 0.00 | 0.00 | 0.00 | 0.02 | 0.01 |
| Gaa^{-/-} 12839scfv:hGAA | 3 | 2.42± 2.41 | 1.63± 0.96 | 0.14± 0.12 | 0.13± 0.12 | 0.19± 0.19 | 0.53± 0.52 | 0.14± 0.16 |
| Gaa^{-/-} 12843scfv:hGAA | 3 | 2.07± 1.35 | 2.23± 0.08 | 0.17± 0.07 | 0.11± 0.05 | 0.17± 0.09 | 0.49± 0.31 | 0.18± 0.06 |
| Gaa^{-/-} 12847scfv:hGAA | 3 | 1.56± 0.71 | 1.40± 0.13 | 0.25± 0.04 | 0.21± 0.09 | 0.42± 0.19 | 0.58± 0.17 | 0.19± 0.08 |

Data were quantified from western blot as arbitrary units (FIG. 21). All values are mean ± SD, n=1-3 per group. ^{∗}Total hGAA protein; ^{∗∗}Mature hGAA protein.

TABLE 36

Quantification of glycogen in tissues of Gaa^{-/-}/Tfrc^{hum} mice treated with liver-depot AAV8 anti-hTfRscfv:hGAA

| Treatment group | Cerebrum | Cerebellum | Spinal Cord | Heart | Quadricep | DRG |
|---|---|---|---|---|---|---|
| Wt Untreated | 0.06±0.04^{∗} | 0.01±0.04^{∗} | 0.05±0.05^{∗} | 0.08±0.02^{∗} | 0.34±0.19^{∗} | 0.018 |
| Gaa-/- Untreated | 2.34±0.58 | 2.51±0.38 | 3.08±0.23 | 25.30±6.06 | 13.05±0.98 | 2.58 |
| Gaa-/- 12839scfv:hGAA | 0.11±0.03^{∗} | 0.46±0.08^{∗} | 0.08±0.10^{∗} | 0.68±0.68^{∗} | 2.15±2.52^{∗} | |
| Gaa-/- 12843scfv:hGAA | 0.09±0.02^{∗} | 0.09±0.08^{∗} | 0.13±0.13^{∗} | 0.09±0.01^{∗} | 1.22±1.39^{∗} | |
| Gaa-/- 12847scfv:hGAA | 0.05±0.01^{∗} | 0.02±0.03^{∗} | 0.20±0.33^{∗} | 0.11±0.11^{∗} | 0.80±0.79^{∗} | 0.066 |
| Treatment group | Cerebrum | Cerebellum | Spinal Cord | Heart | Quadricep | |
| Wt Untreated | 0.043±0.016^{∗} | 0.023±0.008^{∗} | 0.05±0.093^{∗} | 0.083±0.022^{∗} | 0.247±0.045^{∗} | |
| Gaa-/- Untreated | 3.16±0.837 | 3.043±0.932 | 3.22±0.414 | 34.48±5.580 | 11.05±2.192 | |
| Gaa-/- 12799scfv:hGAA | 0.063±0.023^{∗} | 0.107±0.078^{∗} | 0.084±0.019^{∗} | 0.123±0.113^{∗} | 0.725±0.599^{∗} | |

All values are glycogen µg/mg tissue, mean ± SD, n=3-4 per group. One Way ANOVA ^{∗}p<0.0001 vs. Gaa^{-/-} Untreated group. For DRGs, a single value was generated from pooled lumbar DRGs from 5 mice per group.

Rescue of glycogen storage in brain and muscle in Gaa^{-/-}/Tfrc^{hum} mice with AAV8 episomal liver depot anti-hTfRscfv:GAA. We tested three selected anti-hTfRscfv:GAA (12799, 12843, and 12847) in Pompe disease model mice to determine whether hTfRscfv:GAA rescued the glycogen storage phenotype. In this experiment, we performed histology on brain and muscle sections to visualize glycogen in the tissues. We found that all three selected anti-hTfRscfv:GAA reduced glycogen staining in the brain and muscle. We selected 12847scfv:GAA for further analysis based on these data.

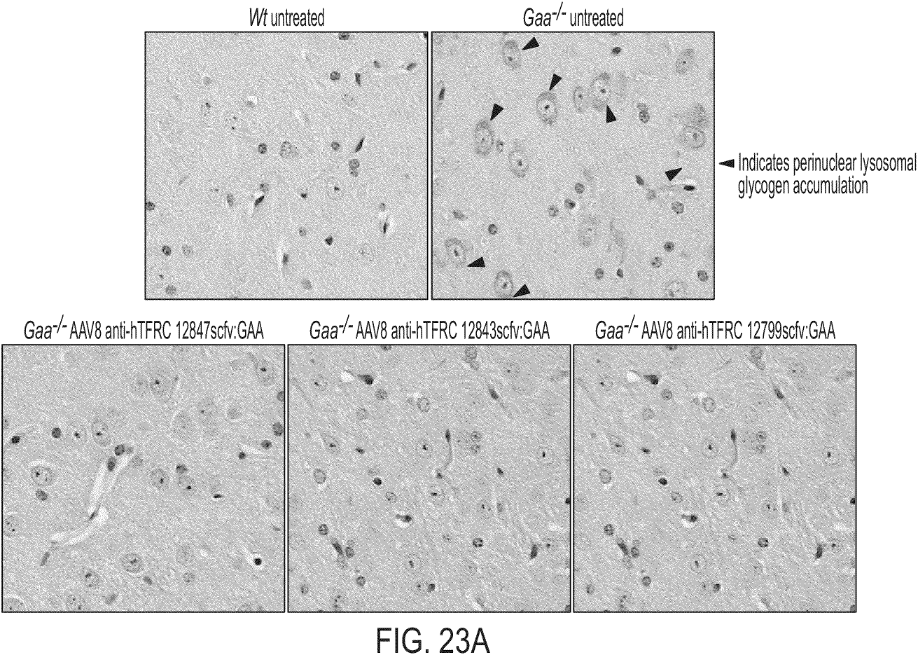

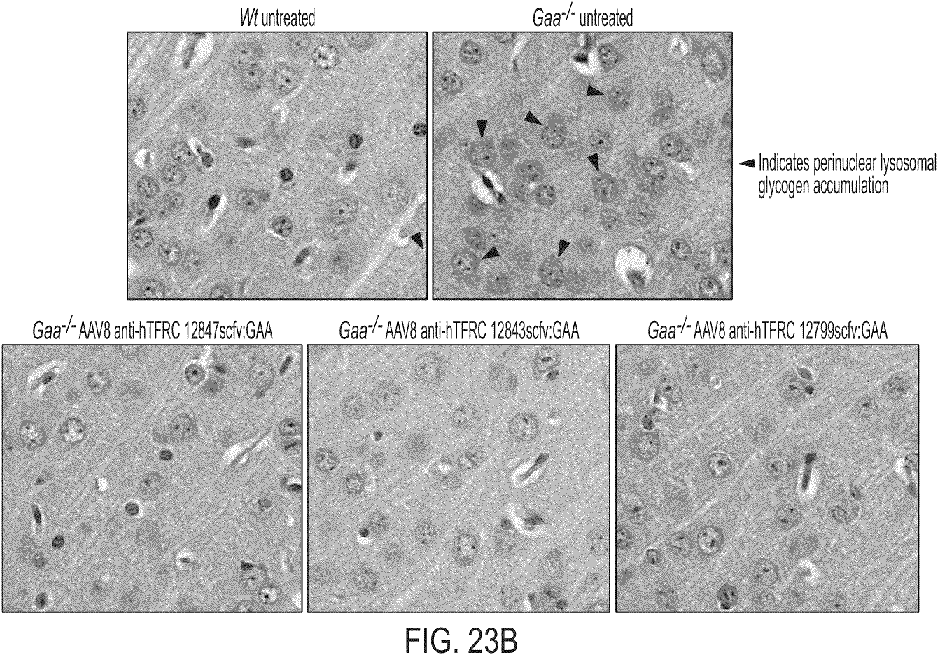

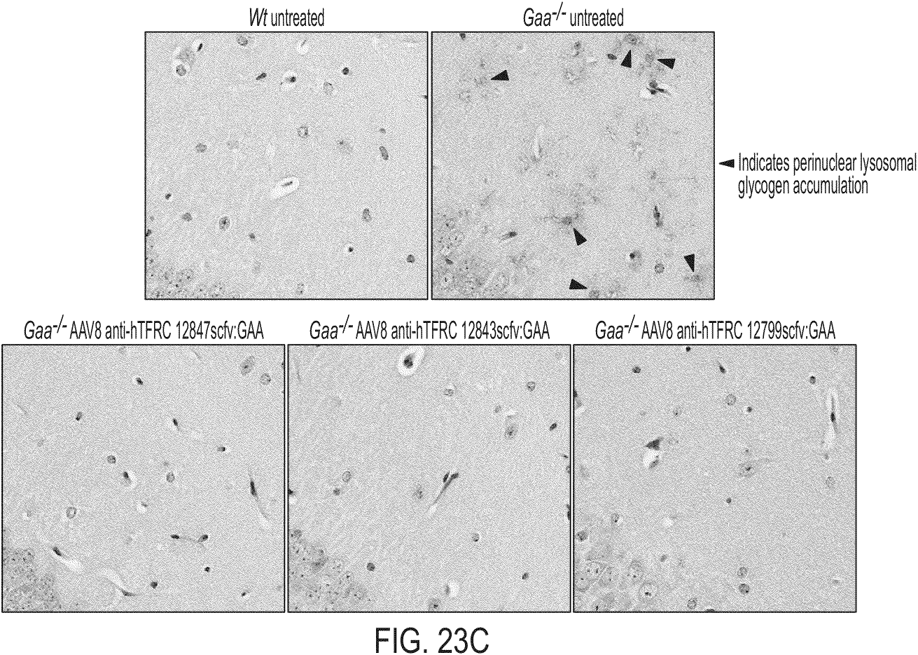

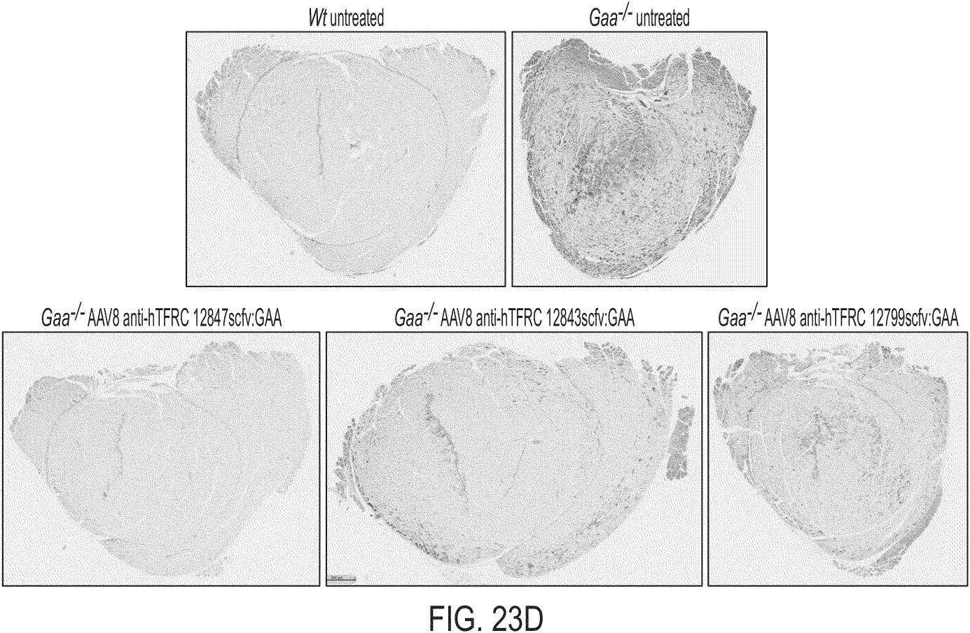

AAV production and in vivo transduction were performed as above. Three-month old Gaa^{-/-}/Tfrc^{hum} mice were dosed with 4e11 vg/kg AAV8. 4 weeks post-injection, tissues were frozen for glycogen analysis as above (Table 37). For histology, animals were perfused with saline (0.9% NaCl), and tissues were drop-fixed overnight in 10% Normal Buffered Formalin. Tissues were washed 3x in PBS and stored in PBS/0.01% sodium azide until embedding. Tissues were embedded in paraffin and 5um sections were cut from brain (coronal, -2 mm bregma) and quadricep (fiber cross-section). Sections were stained with Periodic Acid-Schiff and Hematoxylin using standard protocols (FIGS. 23A-23D).

TABLE 37

Quantification of glycogen in tissues of Gaa^{-/-}/Tfrc^{hum} mice treated with liver-depot AAV8 anti-hTfRscfv:hGAA

| Treatment group | Cerebellum | Quadricep |
|---|---|---|
| Wt Untreated | 0.02±0.03^{∗} | 0.55±0.10^{∗} |
| Gaa-/- Untreated | 1.91±0.26 | 12.19±3.02 |
| Gaa-/- 12799scfv:hGAA | 0.10±0.06^{∗} | 1.34±0.9^{∗} |
| Gaa-/- 12843scfv:hGAA | 0.09±0.06^{∗} | 1.09±1.27^{∗} |
| Gaa-/- 12847scfv:hGAA | 0.07±0.06^{∗} | 0.72±0.64^{∗} |

All values are glycogen µg/mg tissue, mean ± SD, n=5-8 per group. One Way ANOVA ^{∗}p<0.0001 vs. Gaa^{-/-} Untreated group.

Insertion of anti-hTfR 12847scfv:GAA in Gaa^{-/-}/Tfrc^{hum} mice. We tested the selected anti-hTfR 12847scfv:GAA in Pompe disease model mice by albumin insertion to determine whether we could replicate the results we saw with episomal AAV8 liver depot expression. Albumin insertion of 12847scfv:GAA delivered mature hGAA protein to the brain and muscle, and rescued the glycogen storage phenotype in Gaa^{-/-}/Tfrc^{hum} mice. These data were produced with the native 12847scfv:GAA sequence that is not optimized.

We compared 12847scfv:GAA to the muscle-targeted anti-hCD63scfv:GAA in Gaa^{-/-} /Cd63^{hum} mice. In this particular experiment, the expression of anti-hCD63scfv:GAA was lower than usual and does not deliver as much GAA protein to the muscle nor normalize glycogen as it usually does. This may make it appear that anti-hCD63scfv:GAA is less effective than 12847scfv:GAA in the muscle but in most experiments we found them to be comparable in the muscle.

AAVproduction. A promoterless AAV genome plasmid was created with the 12847scfv:GAA sequence and the mouse albumin exon 1 splice acceptor site at the 3′ end. Recombinant AAV8 (AAV2/8) was produced in HEK293 cells. Cells were transfected with three plasmids encoding adenovirus helper genes, AAV8 rep and cap genes, and recombinant AAV genomes containing transgenes flanked by AAV2 inverted terminal repeats (ITRs). On day 5, cells and medium were collected, centrifuged, and processed for AAV purification. Cell pellets were lysed by freeze-thaw and cleared by centrifugation. Processed cell lysates and medium were overlaid onto iodixanol gradients columns and centrifuged in an ultracentrifuge. Virus fractions were removed from the interface between the 40% and 60% iodixanol solutions and exchanged into 1xPBS with desalting columns. AAV vg were quantified by ddPCR.

In vivo CRISPR/Cas9 insertion into the albumin locus. 3-month old Gaa^{-/-}/Tfrc^{hum} mice were dosed via tail vein injection with 3e12 vg/kg AAV8 12847scfv:GAA and 3 mg/kg LNP G666/Cas9 mRNA diluted in PBS + 0.001% F-68 Pluronic. Mice were sacrificed 3 weeks post injection. Negative control mice received insertion AAV8 without LNP. Positive control mice were dosed with 4e11 vg/kg episomal liver depot AAV8 12847scfv:GAA under the TTR promoter (phenotype rescue data previously shown). Tissues were dissected from mice immediately after sacrifice by CO₂ asphyxiation, snap frozen in liquid nitrogen, and stored at -80° C. Blood was collected from mice by cardiac puncture immediately following CO₂ asphyxiation and serum was separated using serum separator tubes (BD Biosciences, 365967).

TABLE 38

Treatment Groups and Controls

| | | |
|---|---|---|
| Treatment group | Genotype | Function |
| Wt Untreated | Tfrc^{hum} | Normal untreated mouse control |
| Gaa^{-/-} untreated | Gaa^{-/-}/ Tfrc^{hum} | Untreated Pompe disease mouse |
| Gaa^{-/-}insertion AAV only | Gaa^{-/-}/ Tfrc^{hum} | Negative control for insertion (no Cas9/gRNA delivered) |
| Gaa^{-/-}episomal AAV8 TTR 12847scfv:hGAA | Gaa^{-/-}/ Tfrc^{hum} | Positive control, previously shown rescue of glycogen storage phenotype |
| Gaa^{-/-} insertion 12847scfv:hGAA | Gaa^{-/-}/ Tfrc^{hum} | Experimental insertion group |
| Gaa^{-/-} untreated | Gaa^{-/-}/ Cd63^{hum} | Untreated Pompe disease mouse (CD63 humanized) |
| Gaa^{-/-} insertion anti-CD63scfv:hGAA | Gaa^{-/-}/ Cd63^{hum} | Negative control for BBB-crossing (muscle targeted) |

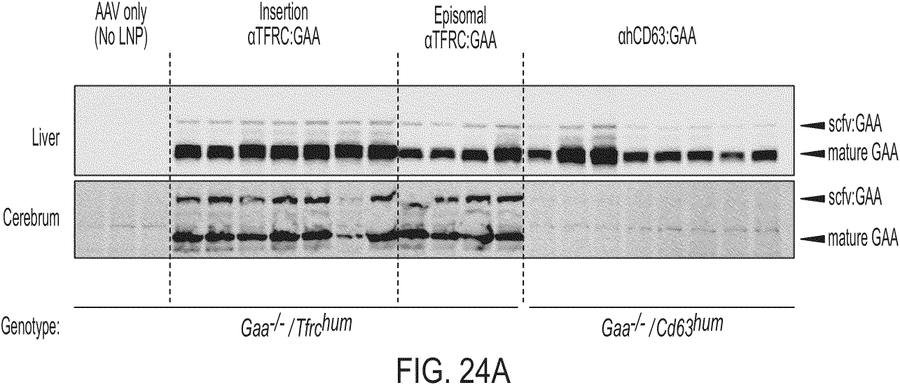

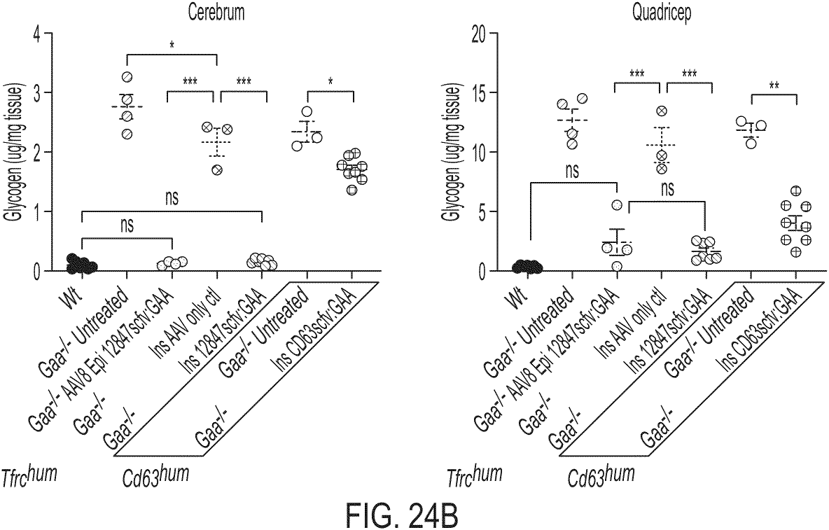

Western blot (Table 39, FIG. 24A). Tissue lysates were prepared by lysis in RIPA buffer with protease inhibitors (1861282, Thermo Fisher, Waltham, MA, USA). Tissue lysates were homogenized with a bead homogenizer (FastPrep5, MP Biomedicals, Santa Ana, CA, USA). Cells or tissue lysates were run on SDS-PAGE gels using the Novex system (LifeTech Thermo, XPO4200BOX, LC2675, LC3675, LC2676). Gels were transferred to low-fluorescence polyvinylidene fluoridev (PVDF) membrane (IPFL07810, LI-COR, Lincoln, NE, USA) and stained with Revert 700 Total Protein Stain (TPS; 926-11010 LI-COR, Lincoln, NE, USA), followed by blocking with Odyssey blocking buffer (927-500000, LI-COR, Lincoln, NE, USA) in Tris buffer saline with 0.1% Tween 20 and staining with antibodies against GAA (ab137068, Abcam, Cambridge, MA, USA), or anti-GAPDH (ab9484, Abcam, Cambridge, MA, USA) and the appropriate secondary (926-32213 or 925-68070, LI-COR, Lincoln, NE, USA). Blots were imaged with a LI-COR Odyssey CLx.

Protein band intensity was quantified in LI-COR Image Studio software. The quantification of the mature 77 kDa GAA band for each sample was determined by normalizing to the lane’s TPS signal (loading control).

Glycogen quantification (Table 40, FIG. 24B). Tissues were dissected from mice immediately after sacrifice by CO₂ asphyxiation, snap frozen in liquid nitrogen, and stored at -80° C. Tissues were lysed on a benchtop homogenizer with stainless steel beads in distilled water for glycogen measurements or RIPA buffer for protein analyses. Glycogen analysis lysates were boiled and centrifuged to clear debris. Glycogen measurements were performed fluorometrically with a commercial kit according to manufacturer’s instructions (K646, BioVision, Milpitas, CA, USA).

TABLE 39

Quantification of hGAA protein in tissues of Gaa^{-/-}/Tfrc^{hum} mice treated with insertion anti-hTfR 12847scfv:hGAA

| Treatment group | Liver total hGAA | Serum total hGAA | Cerebrum mature hGAA | Quadricep mature hGAA |
|---|---|---|---|---|
| Gaa^{-/-} insertion AAV only negative control | 0.02±0.003 | 0.03±0.02 | 0.002±0.001 | 0.006±0.002 |
| Gaa^{-/-} episomal AAV8 TTR 12847scfv:hGAA | 2.35±0.72 | 3.65±2.09 | 0.49±0.20^{§§} | 0.148±0.043^{§§} |
| Gaa^{-/-} insertion 12847scfv:hGAA | 4.31±0.87^{∗} | 3.47±2.37 | 0.57±0.26^{§§} | 0.141±0.062^{§§} |
| Gaa^{-/-} insertion anti-CD63scfv:hGAA | 2.67±1.04^{∗} | 0.93±0.55^{∗} | 0.01±0.003 | 0.060±0.037 |

All values are arbitrary units, mean ± SD, n=3-8 per group. One Way ANOVA ^{∗}p<0.05 vs. Gaa^{-/-} episomal AAV8 TTR 12847scfv:GAA group; ^{§§}p<0.001 vs. AAV only negative control group.

TABLE 40

Quantification of glycogen in tissues of Gaa^{-/-}/Tfrc^{hum} mice treated with insertion anti-hTfR 12847scfv:hGAA

| Treatment group | Cerebrum | Quadricep |
|---|---|---|
| Wt untreated | 0.10±0.07 | 0.37±0.13 |
| Gaa^{-/-}/Tfrc^{hum} untreated (Tfrc^{hum}) | 2.76±0.41 | 12.75±1.88 |
| Gaa^{-/-}/Tfrc^{hum} insertion AAV only | 2.17±0.40^{∗} | 10.64±2.56 |
| Gaa^{-/-}/Tfrc^{hum} episomal AAV8 TTR 12847scfv:hGAA | 0.13+-0.03^{∗∗∗§} | 2.44±2.21^{∗∗∗§} |
| Gaa^{-/-}/Tfr^{hum} insertion 12847scfv:hGAA | 0.16±0.05^{∗∗∗§} | 1.67±0.76^{∗∗∗§} |
| Gaa^{-/-}/Cd63^{hum} untreated | 2.34±0.30 | 11.91±1.01 |
| Gaa^{-/-}/Cd63^{hum} insertion anti-CD63scfv:hGAA | 1.71±0.20^{∗} | 4.06±0.13^{∗∗} |

All values are glycogen µg/mg tissue, mean ± SD, n=3-8 per group. One Way ANOVA ^{∗}p<0.01 vs. Gaa^{-/-}/Cd63^{hum} untreated group; ^{∗∗}p<0.001 vs. Gaa^{-/-}/Cd63^{hum} untreated group; ^{∗∗∗}p<0.0001 vs. Gaa^{-/-}/Tfrc^{hum} untreated group; non-significant vs. Wt untreated group.

Similar experiments are then performed in which neonatal Gaa^{-/-};Tfrc^{hu/hu} mice are dosed intravenously at P1 with the following: (1) recombinant AAV8 encoding anti-TfR:GAA; or (2) LNP-g666 and recombinant AAV8 anti-TfR:GAA insertion template. Untreated Gaa^{-/-} ;Tfrc^{hu/hu} mice and wild type mice are used as controls. Blood is collected and serum prepared at various time points post-administrations, and tissues are collected at various time points post-administration. Serum anti-TfR:GAA levels and glycogen levels in various muscle and CNS tissues are measured over the time course.

To assess whether glycogen reduction translates into improved muscle function, the mice are tested on grip strength apparatuses at a time point post-administration. Limb grip strength is measured with a force meter (Columbus Instruments, Columbus, OH, USA). All tests are performed in triplicate.

In summary, the combination of the highly precise and targeted CRISPR/Cas9 technology delivered by LNP and the anti-TfR:GAA DNA template delivered by the selected rAAV8 vector allows for long-term expression of anti-TfR:GAA protein from hepatocytes and delivery to muscle cells and CNS cells affected in PD, potentially providing a life-long effective treatment to PD patients, including neonatal patients.

### Example 7. Optimized Anti-TfR:GAA DNA Templates

Optimized anti-TfR:GAA templates were designed and generated to develop a lead for non-human primate (NHP) studies. To select a development candidate, several versions of the four candidate anti-TfR:GAA insertion templates were generated in which the nucleotide sequence encoding the anti-TfR:GAA is modified (e.g., by depleting CpGs). Tables 41 and 42 list the different versions of anti-TfR:GAA inserts designed. Each of the anti-TfR:GAA inserts in Table 42 use the optimized GAA sequence set forth in SEQ ID NO: 176.

TABLE 41

Anti-TfR:GAA Inserts for Insertion Cassettes

| Anti-TfR:GAA Insert | CpGs | SEQ ID NO (optimized GAA) |
|---|---|---|
| 12799 - DC | 0 | 205 |
| 12799 - GS | 0 | 206 |
| 12799 - 1^{st} generation | 160 | 174 |
| 12839 - DC | 0 | 207 |
| 12839 - GS | 0 | 208 |
| 12839 - 1^{st} generation | 163 | 174 |
| 12843 - DC | 0 | 209 |
| 12843 - GS | 0 | 210 |
| 12843 - 1^{st} generation | 160 | 174 |
| 12847 - DC | 0 | 211 |
| 12847 - GS | 0 | 212 |
| 12847 - 1^{st} generation | 160 | 174 |

TABLE 42

Anti-TfR:GAA Inserts for Insertion Cassettes

| Anti-TfR:GAA Insert | CpGs in Transgene | SEQ ID NO |
|---|---|---|
| 12799 1^{st} generation | 160 | 574 |
| 12799 GA 0 | 0 | 578 |
| 12799 GS 0 | 0 | 579 |
| 12799 GS 0v2 | 0 | 580 |
| 12843 1^{st} generation | 160 | 576 |
| 12843 GA 0 | 0 | 581 |
| 12843 GS 0 | 0 | 582 |
| 12843 GS 0v2 | 0 | 583 |
| 12847 1^{st} generation | 160 | 577 |
| 12847 GA 0 | 0 | 584 |
| 12847 GS 0 | 0 | 585 |
| 12847 GS 0v2 | 0 | 586 |

Peripheral blood mononuclear cells (PBMCs) are isolated from human blood. Plasmacytoid dendritic cells (pDCs) are enriched and combined with pBMCs (1e4 pDCs + 1e5 PBMCs per well). The cells are incubated for 16-18 hours with AAV or control CpG-oligodeoxynucleotides (ODNs). The supernatants are harvested, and an IFNα ELISA is performed. This assay assesses whether CpG-depleted anti-TfR:GAA sequences exhibit reduced IFN-I responses in a primary human plasmacytoid DC-based assay as compared to non-CpG-depleted sequences.

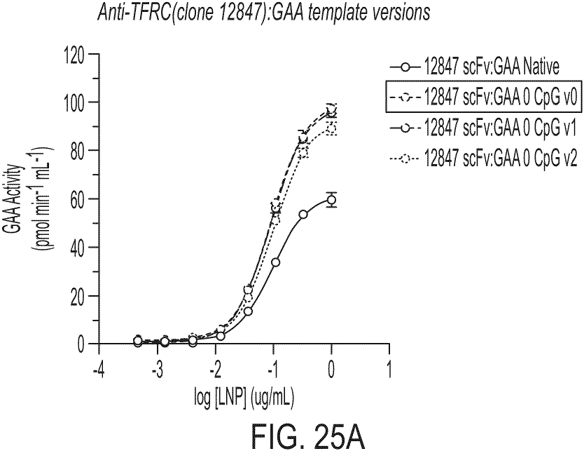

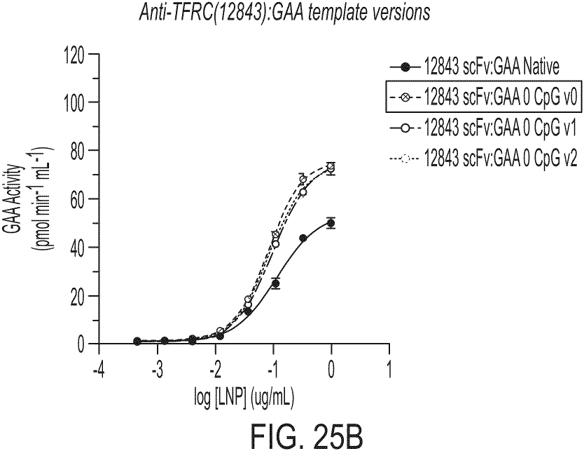

Activity of the various 12847 optimized anti-TfR:GAA templates (SEQ ID NOS: 732-735 or 584-586 (coding sequences)) and 12843 optimized anti-TfR:GAA templates (SEQ ID NOS: 729-731 or 581-583 (coding sequences)) was tested in a primary human hepatocyte assay. AAV templates were packaged into AAV2 viruses. Primary human hepatocytes were grown in 96-well plates and administered the AAV containing the template DNA and LNP-g9860 at fixed MOI (6e4) with LNP dose titration. Supernatants were collected 7 days post-dosing and stored at -80° C. Supernatants were thawed and GAA activity in the supernatants was measured using a 4-methylumbelliferone-based fluorometric assay (K690, BioVision, Milpitas, CA, USA) as a measurement of amount of enzymatically active GAA produced and secreted from the cells. As shown in FIGS. 25A-25B, all CpG-depleted anti-TfR:GAA templates exhibited increased GAA activity in primary human hepatocyte supernatant compared to the native anti-TfR:GAA templates.

Activity of the optimized templates is tested in a primary human hepatocyte assay. AAV templates are packaged into AAV2 viruses. Primary human hepatocytes are grown in 96-well plates and administered the AAV containing the template DNA and LNP-g9860 at fixed LNP concentration with AAV dose titration. Supernatants are collected 7 days post-dosing and stored at -80° C. Supernatants are thawed and GAA activity in the supernatants is measured using a 4-methylumbelliferone-based fluorometric assay (K690, BioVision, Milpitas, CA, USA) as a measurement of amount of enzymatically active GAA produced and secreted from the cells.

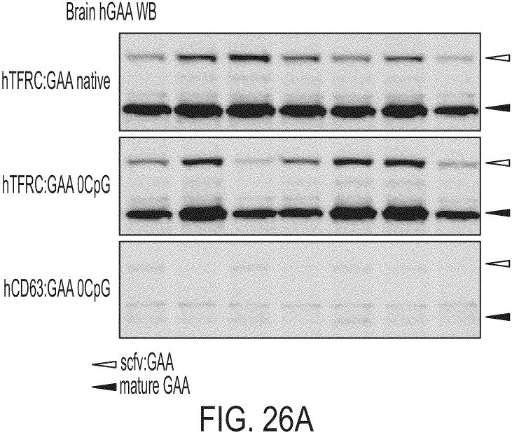

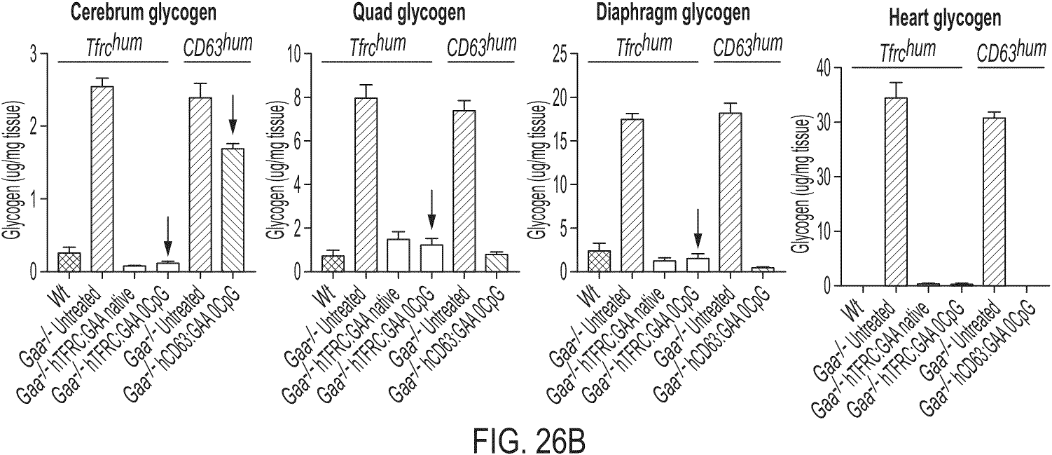

Activity of the 12847 scFv:GAA 0 CpG v0 optimized template (SEQ ID NO: 733 or 584 (coding sequence)) was then validated in the PD mouse model, Gaa^{-/-};Tfrc^{hu/hu}, as described in Example 6. Three-month old mice (Gaa^{-/-};Tfrc^{hu/hu} mice and Gaa^{-/-};CD63^{hu/hu} mice) were dosed intravenously with 3 mg/kg LNP-g9860 and 3ev12 vg/kg AAV8 anti-TfR:GAA templates (native or 12847 0 CpG v0) and optimized anti-CD63:GAA template (GA 0 CpG anti-CD63:GAA template; SEQ ID NO: 736 or 196 (coding sequence)), respectively. Western blots for GAA (scFv:GAA and mature GAA) were done as in Example 6 and confirmed delivery of GAA to the brain (cerebrum) following albumin insertion of the native anti-TfR:GAA template or the 0 CpG anti-TfR:GAA template (FIG. 26A). Glycogen quantification in cerebrum, quadriceps, diaphragm, and heart was also done as in Example 6 and confirmed that albumin insertion of the 0 CpG anti-TfR:GAA templates retained TfR binding and GAA activity in vivo and that the CpG depleted sequence was as effective as the native sequence at rescuing the glycogen storage phenotype in Gaa^{-/-};Tfrc^{hu/hu} mice (FIG. 26B and Table 43).

TABLE 43

Quantification of glycogen in tissues of Gaa^{-/-}/Tfrc^{hum} mice treated with anti-hTfRscfv:hGAA insertion templates

| Treatment group | Cerebrum | Diaphragm | Heart | Quadricep |
|---|---|---|---|---|
| hTFRC Wt | 0.256±0.175^{∗} | 2.44±1.97^{∗} | 0.042±0.022^{∗} | 0.724±0.611^{∗} |
| hTFRC Gaa-/- Untreated | 2.56±0.237 | 17.54±1.72 | 34.56±6.36 | 7.96±1.38 |
| hTFRC Gaa^{-/-} 12847:GAA native | 0.076±0.022^{∗} | 1.30±0.973^{∗} | 0.405±0.351^{∗} | 1.48±0.867^{∗} |
| hTFRC Gaa^{-/-} 12847:GAA 0CpG | 0.114±0.049^{∗} | 1.55±1.60^{∗} | 0.35±0.372^{∗} | 1.21:0.821^{∗} |
| hCD63 Gaa^{-/-} Untreated | 2.39±0.432 | 18.30±2.62 | 30.88±2.451 | 7.37±1.134 |
| hCD63 Gaa^{-/-} CD63:GAA 0CpG | 1.68±0.220^{∗} | 0.544±0.294^{∗} | 0.0634±0.045^{∗} | 0.803±0.297^{∗} |

All values are glycogen µg/mg tissue, mean ± SD, n=5-8 per group. One Way ANOVA ^{∗}p<0.0001 vs. hTFRC Gaa^{-/-} Untreated group.

Expression of the optimized templates (12847 scFv:GAA 0 CpG v0 (SEQ ID NO: 733 or 584 (coding sequence)), and 12843 scFv:GAA 0 CpG v0 (SEQ ID NO: 729 or 581 (coding sequence))) is evaluated in non-human primates. Expression is evaluated by administering LNP-g9860 as described in Example 1 and rAAV8 comprising each optimized template. Expression is analyzed over a multi-week study. Tissues are also collected for analysis of biodistribution of GAA, and GAA activity is assessed in collected tissues.

TABLE 44

Study to evaluate expression of CpG-depleted anti-TfR:GAA insertion templates in non-human primates

| Group | Name | AAV8 Vector | AAV8 Dose | LNP Dose | Guide | n |
|---|---|---|---|---|---|---|
| 1 | Vehicle control | Vehicle only | Vehicle only | Vehicle only | N/A | 1 |
| 2 | Anti-CD63:GAA AAV only control | Anti-CD63:GAA | 1.5e13 vg/kg | Vehicle only | N/A | 1 |
| 3 | Anti-CD63:GAA | Anti-CD63:GAA | 1.5e13 vg/kg | 3 mg/kg LNP | G9860 | 4 |
| 4 | Anti-CD63:GAA alt gRNA | Anti-CD63:GAA | 1.5e13 vg/kg | 3 mg/kg LNP | G9844 | 4 |
| 5 | 12847 Anti-TfR:GAA AAV only control | Anti-TfR:GAA #1 | 1.5e13 vg/kg | Vehicle only | N/A | 1 |
| 6 | 12847 Anti-TfR:GAA | Anti-TfR:GAA #1 | 1.5e13 vg/kg | 3 mg/kg LNP | G9860 | 4 |
| 7 | 12847 Anti-TfR:GAA alt gRNA | Anti-TfR:GAA #1 | 1.5e13 vg/kg | 3 mg/kg LNP | G9844 | 4 |
| 8 | 12843 Anti-TfR:GAA AAV only control | Anti-TfR:GAA #2 | 1.5e13 vg/kg | Vehicle only | N/A | 1 |
| 9 | 12843 Anti-TfR:GAA | Anti-TfR:GAA #2 | 1.5e13 vg/kg | 3 mg/kg LNP | G9860 | 4 |
| 10 | 12843 Anti-TfR:GAA alt gRNA | Anti-TfR:GAA #2 | 1.5e13 vg/kg | 3 mg/kg LNP | G9844 | 4 |
| | | | | | Total animals: | 28 |

TABLE 45

Additional GAA sequences

| | |
|---|---|
| Sequence name | Amino acids |
| GAA_1 77 (SEQ ID NO: 213) | GHILLHDFLLVPRELSGSSPVLEETHPAHQQGASRPGPRDAQAHPGRPRAVPTQCDVPP NSRFDCAPDKAITQEQCEARGCCYIPAKQGLQGAQMGQPWCFFPPSYPSYKLENLSSSE MGYTATLTRTTPTFFPKDILTLRLDVMMETENRLHFTIKDPANRRYEVPLETPHVHSRA PSPLYSVEFSEEPFGVIVRRQLDGRVLLNTTVAPLFFADQFLQLSTSLPSQYITGLAEHLS PLMLSTSWTRITLWNRDLAPTPGANLYGSHPFYLALEDGGSAHGVFLLNSNAMDVVL QPSPALSWRSTGGILDVYIFLGPEPKSVVQQYLDVVGYPFMPPYWGLGFHLCRWGYSS TAITRQVVENMTRAHFPLDVQWNDLDYMDSRRDFTFNKDGFRDFPAMVQELHQGGR RYMMIVDPAISSSGPAGSYRPYDEGLRRGVFITNETGQPLIGKVWPGSTAFPDFTNPTA LAWWEDMVAEFHDQVPFDGMWIDMNEPSNFIRGSEDGCPNNELENPPYVPGVVGGT LQAATICASSHQFLSTHYNLHNLYGLTEAIASHRALVKARGTRPFVISRSTFAGHGRYA GHWTGDVWSSWEQLASSVPEILQFNLLGVPLVGADVCGFLGNTSEELCVRWTQLGAF YPFMRNHNSLLSLPQEPYSFSEPAQQAMRKALTLRYALLPHLYTLFHQAHVAGETVAR PLFLEFPKDSSTWTVDHQLLWGEALLITPVLQAGKAEVTGYFPLGTWYDLQTVPVEAL GSLPPPPAAPREPAIHSEGQWVTLPAPLDTINVHLRAGYIIPLQGPGLTTTESRQQPMAL AVALTKGGEARGELFWDDGESLEVLERGAYTQVIFLARNNTIVNELVRVTSEGAGLQL QKVTVLGVATAPQQVLSNGVPVSNFTYSPDTKVLDICVSLLMGEQFLVSWC |
| GAA_2 22 (SEQ ID NO: 214) | LLVPRELSGSSPVLEETHPAHQQGASRPGPRDAQAHPGRPRAVPTQCDVPPNSRFDCAP DKAITQEQCEARGCCYIPAKQGLQGAQMGQPWCFFPPSYPSYKLENLSSSEMGYTATL TRTTPTFFPKDILTLRLDVMMETENRLHFTIKDPANRRYEVPLETPHVHSRAPSPLYSVE FSEEPFGVIVRRQLDGRVLLNTTVAPLFFADQFLQLSTSLPSQYITGLAEHLSPLMLSTS WTRITLWNRDLAPTPGANLYGSHPFYLALEDGGSAHGVFLLNSNAMDVVLQPSPALS WRSTGGILDVYIFLGPEPKSVVQQYLDVVGYPFMPPYWGLGFHLCRWGYSSTAITRQV VENMTRAHFPLDVQWNDLDYMDSRRDFTFNKDGFRDFPAMVQELHQGGRRYMMIV DPAISSSGPAGSYRPYDEGLRRGVFITNETGQPLIGKVWPGSTAFPDFTNPTALAWWED MVAEFHDQVPFDGMWIDMNEPSNFIRGSEDGCPNNELENPPYVPGVVGGTLQAATICA SSHQFLSTHYNLHNLYGLTEAIASHRALVKARGTRPFVISRSTFAGHGRYAGHWTGDV WSSWEQLASSVPEILQFNLLGVPLVGADVCGFLGNTSEELCVRWTQLGAFYPFMRNH NSLLSLPQEPYSFSEPAQQAMRKALTLRYALLPHLYTLFHQAHVAGETVARPLFLEFPK DSSTWTVDHQLLWGEALLITPVLQAGKAEVTGYFPLGTWYDLQTVPVEALGSLPPPPA APREPAIHSEGQWVTLPAPLDTINVHLRAGYIIPLQGPGLTTTESRQQPMALAVALTKG GEARGELFWDDGESLEVLERGAYTQVIFLARNNTIVNELVRVTSEGAGLQLQKVTVLG VATAPQQVLSNGVPVSNFTYSPDTKVLDICVSLLMGEQFLVSWC |
| GAA_3 61 (SEQ ID NO: 215) | AHPGRPRAVPTQCDVPPNSRFDCAPDKAITQEQCEARGCCYIPAKQGLQGAQMGQPW CFFPPSYPSYKLENLSSSEMGYTATLTRTTPTFFPKDILTLRLDVMMETENRLHFTIKDP ANRRYEVPLETPHVHSRAPSPLYSVEFSEEPFGVIVRRQLDGRVLLNTTVAPLFFADQFL QLSTSLPSQYITGLAEHLSPLMLSTSWTRITLWNRDLAPTPGANLYGSHPFYLALEDGG SAHGVFLLNSNAMDVVLQPSPALSWRSTGGILDVYIFLGPEPKSVVQQYLDVVGYPFM PPYWGLGFHLCRWGYSSTAITRQVVENMTRAHFPLDVQWNDLDYMDSRRDFTFNKD GFRDFPAMVQELHQGGRRYMMIVDPAISSSGPAGSYRPYDEGLRRGVFITNETGQPLIG KVWPGSTAFPDFTNPTALAWWEDMVAEFHDQVPFDGMWIDMNEPSNFIRGSEDGCPN NELENPPYVPGVVGGTLQAATICASSHQFLSTHYNLHNLYGLTEAIASHRALVKARGT RPFVISRSTFAGHGRYAGHWTGDVWSSWEQLASSVPEILQFNLLGVPLVGADVCGFLG NTSEELCVRWTQLGAFYPFMRNHNSLLSLPQEPYSFSEPAQQAMRKALTLRYALLPHL YTLFHQAHVAGETVARPLFLEFPKDSSTWTVDHQLLWGEALLITPVLQAGKAEVTGYF PLGTWYDLQTVPVEALGSLPPPPAAPREPAIHSEGQWVTLPAPLDTINVHLRAGYIIPLQ GPGLTTTESRQQPMALAVALTKGGEARGELFWDDGESLEVLERGAYTQVIFLARNNTI VNELVRVTSEGAGLQLQKVTVLGVATAPQQVLSNGVPVSNFTYSPDTKVLDICVSLLM GEQFLVSWC |

We claim:

1. A composition comprising a nucleic acid construct comprising a coding sequence for a multidomain therapeutic protein comprising a delivery domain fused to a lysosomal alpha-glucosidase, wherein the lysosomal alpha-glucosidase coding sequence is CpG-depleted relative to a wild type lysosomal alpha-glucosidase coding sequence, optionally wherein the delivery domain is a CD63-binding delivery domain or a TfR-binding delivery domain.

2. A composition comprising a nucleic acid construct comprising a coding sequence for a multidomain therapeutic protein comprising a CD63-binding delivery domain fused to a lysosomal alpha-glucosidase, wherein the lysosomal alpha-glucosidase coding sequence is CpG-depleted relative to a wild type lysosomal alpha-glucosidase coding sequence.

3. The composition of claim 1 or 2, wherein the CD63-binding delivery domain is fused to the lysosomal alpha-glucosidase protein via a peptide linker.

4. The composition of any one of claims 1-3, wherein the lysosomal alpha-glucosidase lacks the lysosomal alpha-glucosidase signal peptide and propeptide.

5. The composition of any one of claims 1-4, wherein the lysosomal alpha-glucosidase comprises the sequence set forth in SEQ ID NO: 173.

6. The composition of any one of claims 1-5, wherein the lysosomal alpha-glucosidase consists of the sequence set forth in SEQ ID NO: 173.

7. The composition of any one of claims 1-6, wherein the lysosomal alpha-glucosidase coding sequence is codon-optimized and CpG-depleted.

8. The composition of any one of claims 1-7, wherein the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and 205-212,
optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176.

9. The composition of any one of claims 1-8, wherein the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and 205-212 and encodes a lysosomal alpha-glucosidase protein comprising SEQ ID NO: 173,
optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176 and encodes a lysosomal alpha-glucosidase protein comprising SEQ ID NO: 173.

10. The composition of any one of claims 1-9, wherein the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and 205-212, is codon-optimized and CpG-depleted, and encodes a lysosomal alpha-glucosidase protein comprising SEQ ID NO: 173,
optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176, is codon-optimized and CpG-depleted, and encodes a lysosomal alpha-glucosidase protein comprising SEQ ID NO: 173.

11. The composition of any one of claims 1-10, wherein the lysosomal alpha-glucosidase coding sequence comprises the sequence set forth in any one of SEQ ID NOS: 174-182 and 205-212,
optionally wherein the lysosomal alpha-glucosidase coding sequence comprises the sequence set forth in SEQ ID NO: 176.

12. The composition of any one of claims 1-11, wherein the lysosomal alpha-glucosidase coding sequence consists of the sequence set forth in any one of SEQ ID NOS: 174-182 and 205-212,
optionally wherein the lysosomal alpha-glucosidase coding sequence consists of the sequence set forth in SEQ ID NO: 176.

13. The composition of any one of claims 1-12, wherein the coding sequence for the CD63-binding delivery domain is codon-optimized or CpG-depleted.

14. The composition of any one of claims 1-13, wherein the coding sequence for the CD63-binding delivery domain is codon-optimized and CpG-depleted.

15. The composition of any one of claims 1-14, wherein the CD63-binding delivery domain comprises an anti-CD63 antigen-binding protein.

16. The composition of any one of claims 1-15, wherein the CD63-binding delivery domain comprises an anti-CD63 antibody, antibody fragment, or single-chain variable fragment (scFv).

17. The composition of claim 16, wherein the CD63-binding delivery domain is the single-chain variable fragment (scFv).

18. The composition of claim 17, wherein the scFv comprises the sequence set forth in SEQ ID NO: 183.

19. The composition of claim 17 or 18, wherein the scFv consists of the sequence set forth in SEQ ID NO: 183.

20. The composition of any one of claims 1-19, wherein the scFv coding sequence is at least 90%, at least 91%, at least 92%, at least 93%, at least 94%, at least 95%, at least 96%, at least 97%, at least 98%, or at least 99% identical to any one of SEQ ID NOS: 184-192,
optionally wherein the scFv coding sequence is at least 90%, at least 91%, at least 92%, at least 93%, at least 94%, at least 95%, at least 96%, at least 97%, at least 98%, or at least 99% identical to SEQ ID NO: 186.

21. The composition of any one of claims 1-20, wherein the scFv coding sequence is at least 90%, at least 91%, at least 92%, at least 93%, at least 94%, at least 95%, at least 96%, at least 97%, at least 98%, or at least 99% identical to any one of SEQ ID NOS: 184-192 and encodes an scFv comprising SEQ ID NO: 183,
optionally wherein the scFv coding sequence is at least 90%, at least 91%, at least 92%, at least 93%, at least 94%, at least 95%, at least 96%, at least 97%, at least 98%, or at least 99% identical to SEQ ID NO: 186 and encodes an scFv comprising SEQ ID NO: 183.

22. The composition of any one of claims 1-21, wherein the scFv coding sequence is at least 90%, at least 91%, at least 92%, at least 93%, at least 94%, at least 95%, at least 96%, at least 97%, at least 98%, or at least 99% identical to any one of SEQ ID NOS: 184-192, is codon-optimized and CpG-depleted, and encodes an scFv comprising SEQ ID NO: 183,
optionally wherein the scFv coding sequence is at least 90%, at least 91%, at least 92%, at least 93%, at least 94%, at least 95%, at least 96%, at least 97%, at least 98%, or at least 99% identical to SEQ ID NO: 186, is codon-optimized and CpG-depleted, and encodes an scFv comprising SEQ ID NO: 183.

23. The composition of any one of claims 1-22, wherein the scFv coding sequence comprises the sequence set forth in any one of SEQ ID NOS: 184-192,
optionally wherein the scFv coding sequence comprises the sequence set forth in SEQ ID NO: 186.

24. The composition of any one of claims 1-23, wherein the scFv coding sequence consists of the sequence set forth in any one of SEQ ID NOS: 184-192,
optionally wherein the scFv coding sequence consists of the sequence set forth in SEQ ID NO: 186.

25. The composition of any one of claims 1-24, wherein the coding sequence for the multidomain therapeutic protein is codon-optimized or CpG-depleted.

26. The composition of any one of claims 1-25, wherein the coding sequence for the multidomain therapeutic protein is codon-optimized and CpG-depleted.

27. The composition of any one of claims 1-26, wherein the multidomain therapeutic protein comprises the sequence set forth in SEQ ID NO: 193.

28. The composition of any one of claims 1-27, wherein the multidomain therapeutic protein consists of the sequence set forth in SEQ ID NO: 193.

29. The composition of any one of claims 1-28, wherein the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 194-202,
optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 196,
optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 736.

30. The composition of any one of claims 1-29, wherein the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 194-202, and the multidomain therapeutic protein comprises the sequence set forth in SEQ ID NO: 193,
optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 196, and the multidomain therapeutic protein comprises the sequence set forth in SEQ ID NO: 193,
optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 736, and the multidomain therapeutic protein comprises the sequence set forth in SEQ ID NO: 193.

31. The composition of any one of claims 1-30, wherein the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 194-202 and is codon-optimized and CpG-depleted, and the multidomain therapeutic protein comprises the sequence set forth in SEQ ID NO: 193,
optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 196 and is codon-optimized and CpG-depleted, and the multidomain therapeutic protein comprises the sequence set forth in SEQ ID NO: 193,
optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 736, the coding sequence for the multidomain therapeutic protein is codon-optimized and CpG-depleted, and the multidomain therapeutic protein comprises the sequence set forth in SEQ ID NO: 193.

32. The composition of any one of claims 1-31, wherein the coding sequence for the multidomain therapeutic protein comprises the sequence set forth in any one of SEQ ID NOS: 194-202,
optionally wherein the coding sequence for the multidomain therapeutic protein comprises the sequence set forth in SEQ ID NO: 196,
optionally wherein the nucleic acid construct comprises the sequence set forth in SEQ ID NO: 736.

33. The composition of any one of claims 1-32, wherein the coding sequence for the multidomain therapeutic protein consists of the sequence set forth in any one of SEQ ID NOS: 194-202,
optionally wherein the coding sequence for the multidomain therapeutic protein consists of the sequence set forth in SEQ ID NO: 196,
optionally wherein the nucleic acid construct comprises the sequence set forth in SEQ ID NO: 736.

34. The composition of any one of claims 1-33, wherein the nucleic acid construct comprises a splice acceptor upstream of the coding sequence for the multidomain therapeutic protein.

35. The composition of any one of claims 1-34, wherein the nucleic acid construct comprises a polyadenylation signal or sequence downstream of the coding sequence for the multidomain therapeutic protein.

36. The composition of any one of claims 1-35, wherein the nucleic acid construct comprises a splice acceptor upstream of the coding sequence for the multidomain therapeutic protein, and the nucleic acid construct comprises a polyadenylation signal or sequence downstream of the coding sequence for the multidomain therapeutic protein.

37. The composition of any one of claims 1-36, wherein the nucleic acid construct does not comprise a homology arm.

38. The composition of any one of claims 1-36, wherein the nucleic acid construct comprises homology arms.

39. The composition of any one of claims 1-38, wherein the nucleic acid construct does not comprise a promoter that drives the expression of the multidomain therapeutic protein.

40. The composition of any one of claims 1-38, wherein the coding sequence for the multidomain therapeutic protein is operably linked to a promoter, optionally wherein the promoter is a liver-specific promoter.

41. The composition of any one of claims 1-40, wherein the nucleic acid construct is single-stranded DNA or double-stranded DNA, optionally wherein the nucleic acid construct is single-stranded DNA.

42. The composition of any one of claims 1-41, wherein the nucleic acid construct comprises from 5′ to 3′: a splice acceptor, the coding sequence for the multidomain therapeutic protein, and a polyadenylation signal or sequence,
wherein the coding sequence for the multidomain therapeutic protein comprises any one of SEQ ID NOS: 194-202, optionally wherein the coding sequence for the multidomain therapeutic protein comprises the sequence set forth in SEQ ID NO: 196, optionally wherein the nucleic acid construct comprises the sequence set forth in SEQ ID NO: 736,
wherein the nucleic acid construct does not comprise a promoter that drives the expression of the multidomain therapeutic protein, and
wherein the nucleic acid construct does not comprise a homology arm.

43. The composition of any one of claims 1-42, wherein the nucleic acid construct is in a nucleic acid vector or a lipid nanoparticle.

44. The composition of claim 43, wherein the nucleic acid construct is in the nucleic acid vector.

45. The composition of claim 44, wherein the nucleic acid vector is a viral vector.

46. The composition of claim 43 or 44, wherein the nucleic acid vector is an adeno-associated viral (AAV) vector,
optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

47. The composition of claim 46, wherein the AAV vector is a single-stranded AAV (ssAAV) vector.

48. The composition of claim 46 or 47, wherein the AAV vector is derived from an AAV8 vector, an AAV3B vector, an AAV5 vector, an AAV6 vector, an AAV7 vector, an AAV9 vector, an AAVrh.74 vector, or an AAVhu.37 vector.

49. The composition of claim 48, wherein the AAV vector is a recombinant AAV8 (rAAV8) vector.

50. The composition of claim 49, wherein the AAV vector is a single-stranded rAAV8 vector.

51. The composition of any one of claims 1-50, wherein the nucleic acid construct comprises from 5′ to 3′: a splice acceptor, the coding sequence for the multidomain therapeutic protein, and a polyadenylation signal or sequence,
wherein the coding sequence for the multidomain therapeutic protein comprises any one of SEQ ID NOS: 194-202, optionally wherein the coding sequence for the multidomain therapeutic protein comprises the sequence set forth in SEQ ID NO: 196, optionally wherein the nucleic acid construct comprises the sequence set forth in SEQ ID NO: 736,
wherein the nucleic acid construct does not comprise a promoter that drives the expression of the multidomain therapeutic protein,
wherein the nucleic acid construct does not comprise a homology arm, and
wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

52. The composition of any one of claims 1-51, wherein the nucleic acid construct is CpG-depleted.

53. The composition of any one of claims 1-51, further comprising a nuclease agent that targets a nuclease target site in a target genomic locus.

54. The composition of claim 53, wherein the target genomic locus is an albumin gene, optionally wherein the albumin gene is a human albumin gene.

55. The composition of claim 54, wherein the nuclease target site is in intron 1 of the albumin gene.

56. The composition of any one of claims 53-55, wherein the nuclease agent comprises:
(a) a zinc finger nuclease (ZFN);
(b) a transcription activator-like effector nuclease (TALEN); or
(c) (i) a Cas protein or a nucleic acid encoding the Cas protein; and
(ii) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

57. The composition of any one of claims 53-55, wherein the nuclease agent comprises:
(a) a Cas protein or a nucleic acid encoding the Cas protein; and
(b) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

58. The composition of claim 57, wherein the guide RNA target sequence is in intron 1 of an albumin gene.

59. The composition of claim 58, wherein the albumin gene is a human albumin gene.

60. The composition of any one of claims 57-59, wherein:
(I) the DNA-targeting segment comprises at least 17, at least 18, at least 19, or at least 20 contiguous nucleotides of the sequence set forth in any one of SEQ ID NOS: 30-61, optionally wherein the DNA-targeting segment comprises at least 17, at least 18, at least 19, or at least 20 contiguous nucleotides of the sequence set forth in any one of SEQ ID NOS: 36, 30, 33, and 41; and/or
(II) the DNA-targeting segment is at least 90% or at least 95% identical to the sequence set forth in any one of SEQ ID NOS: 30-61, optionally wherein the DNA-targeting segment is at least 90% or at least 95% identical to the sequence set forth in any one of SEQ ID NOS: 36, 30, 33, and 41.

61. The composition of any one of claims 57-60, wherein the DNA-targeting segment comprises any one of SEQ ID NOS: 30-61, optionally wherein the DNA-targeting segment comprises any one of SEQ ID NOS: 36, 30, 33, and 41.

62. The composition of any one of claims 57-61, wherein the DNA-targeting segment consists of any one of SEQ ID NOS: 30-61, optionally wherein the DNA-targeting segment consists of any one of SEQ ID NOS: 36, 30, 33, and 41.

63. The composition of any one of claims 57-62, wherein the guide RNA comprises any one of SEQ ID NOS: 62-125, optionally wherein the guide RNA comprises any one of SEQ ID NOS: 68, 100, 62, 94, 65, 97, 73, and 105.

64. The composition of any one of claims 57-63, wherein:
(I) the DNA-targeting segment comprises at least 17, at least 18, at least 19, or at least 20 contiguous nucleotides of SEQ ID NO: 36; and/or
(II) the DNA-targeting segment is at least 90% or at least 95% identical to SEQ ID NO: 36.

65. The composition of any one of claims 57-64, wherein the DNA-targeting segment comprises SEQ ID NO: 36.

66. The composition of any one of claims 57-65, wherein the DNA-targeting segment consists of SEQ ID NO: 36.

67. The composition of any one of claims 57-66, wherein the guide RNA comprises SEQ ID NO: 68 or 100.

68. The composition of any one of claims 57-67, wherein the guide RNA in the form of RNA.

69. The composition of any one of claims 57-68, wherein the guide RNA comprises at least one modification.

70. The composition of claim 69, wherein the at least one modification comprises a 2′-O-methyl-modified nucleotide.

71. The composition of claim 69 or 70, wherein the at least one modification comprises a phosphorothioate bond between nucleotides.

72. The composition of any one of claims 69-71, wherein the at least one modification comprises a modification at one or more of the first five nucleotides at the 5′ end of the guide RNA.

73. The composition of any one of claims 69-72, wherein the at least one modification comprises a modification at one or more of the last five nucleotides at the 3′ end of the guide RNA.

74. The composition of any one of claims 69-73, wherein the at least one modification comprises phosphorothioate bonds between the first four nucleotides at the 5′ end of the guide RNA.

75. The composition of any one of claims 69-74, wherein the at least one modification comprises phosphorothioate bonds between the last four nucleotides at the 3′ end of the guide RNA.

76. The composition of any one of claims 69-75, wherein the at least one modification comprises 2′-O-methyl-modified nucleotides at the first three nucleotides at the 5′ end of the guide RNA.

77. The composition of any one of claims 69-76, wherein the at least one modification comprises 2′-O-methyl-modified nucleotides at the last three nucleotides at the 3′ end of the guide RNA.

78. The composition of any one of claims 69-77, wherein the at least one modification comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

79. The composition of any one of claims 69-78, wherein the guide RNA is a single guide RNA (sgRNA).

80. The composition of any one of claims 69-79, wherein the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

81. The composition of any one of claims 57-80, wherein the Cas protein is a Cas9 protein.

82. The composition of claim 81, wherein the Cas9 protein is derived from a Streptococcus pyogenes Cas9 protein, a Staphylococcus aureus Cas9 protein, a Campylobacter jejuni Cas9 protein, a Streptococcus thermophilus Cas9 protein, or a Neisseria meningitidis Cas9 protein.

83. The composition of claim 81, wherein the Cas protein is derived from a Streptococcus pyogenes Cas9 protein.

84. The composition of any one of claims 57-83, wherein the Cas protein comprises the sequence set forth in SEQ ID NO: 11.

85. The composition of any one of claims 57-84, wherein the nucleic acid encoding the Cas protein is codon-optimized for expression in a mammalian cell or a human cell.

86. The composition of any one of claims 57-85, wherein the composition comprises the nucleic acid encoding the Cas protein, wherein the nucleic acid comprises an mRNA encoding the Cas protein.

87. The composition of claim 86, wherein the mRNA encoding the Cas protein comprises at least one modification.

88. The composition of claim 86 or 87, wherein the mRNA encoding the Cas protein is modified to comprise a modified uridine at one or more or all uridine positions.

89. The composition of claim 88, wherein the modified uridine is pseudouridine or N1-methyl-pseudouridine, optionally N1-methyl-pseudouridine.

90. The composition of claim 88 or 89, wherein the mRNA encoding the Cas protein is fully substituted with pseudouridine or N1-methyl-pseudouridine, optionally N1-methyl-pseudouridine.

91. The composition of any one of claims 86-90, wherein the mRNA encoding the Cas protein comprises a 5′ cap.

92. The composition of any one of claims 86-91, wherein the mRNA encoding the Cas protein comprises a polyadenylation sequence.

93. The composition of any one of claims 86-92, wherein the mRNA encoding the Cas protein comprises the sequence set forth in SEQ ID NO: 226, 225, or 12.

94. The composition of any one of claims 57-93, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine or N1-methyl-pseudouridine, optionally N1-methyl-pseudouridine, comprises a 5′ cap, and comprises a polyadenylation sequence.

95. The composition of any one of claims 57-94, wherein the guide RNA in the form of RNA, and the guide RNA comprises SEQ ID NO: 68 or 100, and
wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 226, 225, or 12.

96. The composition of claim 95, wherein the nucleic acid construct comprises from 5′ to 3′: a splice acceptor, the coding sequence for the multidomain therapeutic protein, and a polyadenylation signal or sequence,
wherein the coding sequence for the multidomain therapeutic protein comprises any one of SEQ ID NOS: 194-202, optionally wherein the coding sequence for the multidomain therapeutic protein comprises the sequence set forth in SEQ ID NO: 196, optionally wherein the nucleic acid construct comprises the sequence set forth in SEQ ID NO: 736,
wherein the nucleic acid construct does not comprise a promoter that drives the expression of the multidomain therapeutic protein,
wherein the nucleic acid construct does not comprise a homology arm, and
wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

97. The composition of any one of claims 57-94, wherein the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and
wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine or N1-methyl-pseudouridine, optionally N1-methyl-pseudouridine, comprises a 5′ cap, and comprises a polyadenylation sequence.

98. The composition of claim 97, wherein the nucleic acid construct comprises from 5′ to 3′: a splice acceptor, the coding sequence for the multidomain therapeutic protein, and a polyadenylation signal or sequence,
wherein the coding sequence for the multidomain therapeutic protein comprises any one of SEQ ID NOS: 194-202, optionally wherein the coding sequence for the multidomain therapeutic protein comprises the sequence set forth in SEQ ID NO: 196, optionally wherein the nucleic acid construct comprises the sequence set forth in SEQ ID NO: 736,
wherein the nucleic acid construct does not comprise a promoter that drives the expression of the multidomain therapeutic protein,
wherein the nucleic acid construct does not comprise a homology arm, and
wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

99. The composition of any one of claims 57-98, wherein the Cas protein or the nucleic acid encoding the Cas protein and the guide RNA or the one or more DNAs encoding the guide RNA are associated with a lipid nanoparticle.

100. The composition of claim 99, wherein the lipid nanoparticle comprises a cationic lipid, a neutral lipid, a helper lipid, and a stealth lipid.

101. The composition of claim 100, wherein the cationic lipid is Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate).

102. The composition of claim 100 or 101, wherein the neutral lipid is distearoylphosphatidylcholine or 1,2-distearoyl-sn-glycero-3-phosphocholine (DSPC).

103. The composition of any one of claims 100-102, wherein the helper lipid is cholesterol.

104. The composition of any one of claims 100-103, wherein the stealth lipid is 1,2-dimyristoyl-rac-glycero-3-methoxypolyethylene glycol-2000 (PEG2k-DMG).

105. The composition of any one of claims 100-104, wherein the cationic lipid is Lipid A, the neutral lipid is DSPC, the helper lipid is cholesterol, and the stealth lipid is PEG2k-DMG.

106. The composition of any one of claims 99-105, wherein the lipid nanoparticle comprises four lipids at the following molar ratios: about 50 mol% Lipid A, about 9 mol% DSPC, about 38 mol% cholesterol, and about 3 mol% PEG2k-DMG.

107. The composition of any one of claims 99-106, wherein the albumin gene is a human albumin gene,
wherein the guide RNA in the form of RNA, and the guide RNA comprises SEQ ID NO: 68 or 100,
wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 226, 225, or 12, and
wherein the guide RNA and the mRNA encoding the Cas protein are associated with a lipid nanoparticle comprising Lipid A, DSPC, cholesterol, and PEG2k-DMG, optionally at the following molar ratios: about 50 mol% Lipid A, about 9 mol% DSPC, about 38 mol% cholesterol, and about 3 mol% PEG2k-DMG.

108. The composition of claim 107, wherein the nucleic acid construct comprises from 5′ to 3′: a splice acceptor, the coding sequence for the multidomain therapeutic protein, and a polyadenylation signal or sequence,
wherein the coding sequence for the multidomain therapeutic protein comprises any one of SEQ ID NOS: 194-202, optionally wherein the coding sequence for the multidomain therapeutic protein comprises the sequence set forth in SEQ ID NO: 196, optionally wherein the nucleic acid construct comprises the sequence set forth in SEQ ID NO: 736,
wherein the nucleic acid construct does not comprise a promoter that drives the expression of the multidomain therapeutic protein,
wherein the nucleic acid construct does not comprise a homology arm, and
wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

109. The composition of any one of claims 57-106, wherein the albumin gene is a human albumin gene,
wherein the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA,
wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine or N1-methyl-pseudouridine, optionally N1-methyl-pseudouridine, comprises a 5′ cap, and comprises a polyadenylation sequence, and
wherein the guide RNA and the mRNA encoding the Cas protein are associated with a lipid nanoparticle comprising Lipid A, DSPC, cholesterol, and PEG2k-DMG, optionally at the following molar ratios: about 50 mol% Lipid A, about 9 mol% DSPC, about 38 mol% cholesterol, and about 3 mol% PEG2k-DMG.

110. The composition of claim 109, wherein the nucleic acid construct comprises from 5′ to 3′: a splice acceptor, the coding sequence for the multidomain therapeutic protein, and a polyadenylation signal or sequence,
wherein the coding sequence for the multidomain therapeutic protein comprises any one of SEQ ID NOS: 194-202, optionally wherein the coding sequence for the multidomain therapeutic protein comprises the sequence set forth in SEQ ID NO: 196, optionally wherein the nucleic acid construct comprises the sequence set forth in SEQ ID NO: 736,
wherein the nucleic acid construct does not comprise a promoter that drives the expression of the multidomain therapeutic protein,
wherein the nucleic acid construct does not comprise a homology arm, and
wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

111. The composition of any one of claims 1-110, for use in a method of inserting the nucleic acid encoding the multidomain therapeutic protein into a target genomic locus in a cell or a population of cells.

112. The composition of any one of claims 1-110, for use in a method of expressing the multidomain therapeutic protein from a target genomic locus in a cell or a population of cells or for use in a method of expressing the multidomain therapeutic protein in a cell or a population of cells.

113. The composition of any one of claims 1-110, for use in a method of inserting the nucleic acid encoding the multidomain therapeutic protein into a target genomic locus in a cell or a population of cells in a subject.

114. The composition of any one of claims 1-110, for use in a method of expressing the multidomain therapeutic protein from a target genomic locus in a cell or a population of cells in a subject or for use in a method of expressing the multidomain therapeutic protein in a cell or a population of cells in a subject.

115. The composition of any one of claims 1-110, for use in a method of treating a lysosomal alpha-glucosidase deficiency in a subject in need thereof.

116. The composition of any one of claims 1-110, for use in a method of reducing glycogen accumulation in a tissue in a subject in need thereof.

117. The composition of any one of claims 1-110, for use in a method of treating Pompe disease in a subject in need thereof.

118. The composition of any one of claims 1-110, for use in a method of preventing or reducing the onset of a sign or symptom of Pompe disease in a neonatal subject in need thereof.

119. A cell comprising the composition of any one of claims 1-110.

120. The cell of claim 119, wherein the nucleic acid construct is integrated into a target genomic locus, and wherein the multidomain therapeutic protein is expressed from the target genomic locus, or wherein the nucleic acid construct is integrated into intron 1 of an endogenous albumin locus, and wherein the multidomain therapeutic protein is expressed from the endogenous albumin locus.

121. The cell of claim 119 or 120, wherein the cell is a liver cell.

122. The cell of claim 121, wherein the liver cell is a hepatocyte.

123. The cell of any one of claims 119-122, wherein the cell is a human cell.

124. The cell of any one of claims 119-123, wherein the cell is a neonatal cell.

125. The cell of claim 124, wherein the neonatal cell is from a human neonatal subject within 24 weeks after birth.

126. The cell of claim 124, wherein the neonatal cell is from a human neonatal subject within 12 weeks after birth.

127. The cell of claim 124, wherein the neonatal cell is from a human neonatal subject within 8 weeks after birth.

128. The cell of claim 124, wherein the neonatal cell is from a human neonatal subject within 4 weeks after birth.

129. The cell of any one of claims 119-123, wherein the cell is not a neonatal cell.

130. The cell of any one of claims 119-129, wherein the cell is in vivo.

131. The cell of any one of claims 119-129, wherein the cell is in vitro or ex vivo.

132. A method of inserting a nucleic acid encoding a multidomain therapeutic protein comprising a delivery domain fused to a lysosomal alpha-glucosidase into a target genomic locus in a cell or a population of cells, comprising administering to the cell or the population of cells the composition of any one of claims 53-110, optionally wherein the delivery domain is a CD63-binding delivery domain or a TfR-binding delivery domain,
wherein the nuclease agent cleaves the nuclease target site in the target genomic locus, and the nucleic acid construct is inserted into the target genomic locus.

133. A method of inserting a nucleic acid encoding a multidomain therapeutic protein comprising a CD63-binding delivery domain fused to a lysosomal alpha-glucosidase into a target genomic locus in a cell or a population of cells, comprising administering to the cell or the population of cells the composition of any one of claims 53-110,
wherein the nuclease agent cleaves the nuclease target site in the target genomic locus, and the nucleic acid construct is inserted into the target genomic locus.

134. A method of expressing a multidomain therapeutic protein comprising a delivery domain fused to a lysosomal alpha-glucosidase in a cell or a population of cells, comprising administering to the cell or the population of cells the composition of any one of claims 1-52, optionally wherein the delivery domain is a CD63-binding delivery domain or a TfR-binding delivery domain,
wherein the coding sequence for the multidomain therapeutic protein is operably linked to a promoter in the nucleic acid construct and is expressed in the cell or population of cells.

135. A method of expressing a multidomain therapeutic protein comprising a delivery domain fused to a lysosomal alpha-glucosidase from a target genomic locus in a cell or a population of cells, comprising administering to the cell or the population of cells the composition of any one of claims 53-110, optionally wherein the delivery domain is a CD63-binding delivery domain or a TfR-binding delivery domain,
wherein the nuclease agent cleaves the nuclease target site in the target genomic locus, the nucleic acid construct is inserted into the target genomic locus to create a modified target genomic locus, and the multidomain therapeutic protein comprising the delivery domain fused to the lysosomal alpha-glucosidase is expressed from the modified target genomic locus.

136. A method of expressing a multidomain therapeutic protein comprising a CD63-binding delivery domain fused to a lysosomal alpha-glucosidase in a cell or a population of cells, comprising administering to the cell or the population of cells the composition of any one of claims 1-52,
wherein the coding sequence for the multidomain therapeutic protein is operably linked to a promoter in the nucleic acid construct and is expressed in the cell or population of cells.

137. A method of expressing a multidomain therapeutic protein comprising a CD63-binding delivery domain fused to a lysosomal alpha-glucosidase from a target genomic locus in a cell or a population of cells, comprising administering to the cell or the population of cells the composition of any one of claims 53-110,
wherein the nuclease agent cleaves the nuclease target site in the target genomic locus, the nucleic acid construct is inserted into the target genomic locus to create a modified target genomic locus, and the multidomain therapeutic protein comprising the CD63-binding delivery domain fused to the lysosomal alpha-glucosidase is expressed from the modified target genomic locus.

138. The method of any one of claims 132-137, wherein the cell is a liver cell or the population of cells is a population of liver cells.

139. The method of any one of claims 132-138, wherein the cell is a hepatocyte or the population of cells is a population of hepatocytes.

140. The method of any one of claims 132-139, wherein the cell is a human cell or the population of cells is a population of human cells.

141. The method of any one of claims 132-140, wherein the cell is a neonatal cell or the population of cells is a population of neonatal cells.

142. The method of claim 141, wherein the neonatal cell or the population of neonatal cells is from a human neonatal subject within 24 weeks after birth, optionally wherein the neonatal cell or the population of neonatal cells is from a human neonatal subject within 12 weeks after birth.

143. The method of claim 141, wherein the neonatal cell or the population of neonatal cells is from a human neonatal subject within 8 weeks after birth, optionally wherein the neonatal cell or the population of neonatal cells is from a human neonatal subject within 4 weeks after birth.

144. The method of any one of claims 132-140, wherein the cell is not a neonatal cell or the population of cells is not a population of neonatal cells.

145. The method of any one of claims 132-144, wherein the cell is in vitro or ex vivo or the population of cells is in vitro or ex vivo.

146. The method of any one of claims 132-144, wherein the cell is in vivo in a subject or the population of cells is in vivo in a subject.

147. A method of inserting a nucleic acid encoding a multidomain therapeutic protein comprising a delivery domain fused to a lysosomal alpha-glucosidase into a target genomic locus in a cell in a subject, comprising administering to the subject the composition of any one of claims 53-110, optionally wherein the delivery domain is a CD63-binding delivery domain or a TfR-binding delivery domain,
wherein the nuclease agent cleaves the nuclease target site in the target genomic locus, and the nucleic acid construct is inserted into the target genomic locus.

148. A method of inserting a nucleic acid encoding a multidomain therapeutic protein comprising a CD63-binding delivery domain fused to a lysosomal alpha-glucosidase into a target genomic locus in a cell in a subject, comprising administering to the subject the composition of any one of claims 53-110,
wherein the nuclease agent cleaves the nuclease target site in the target genomic locus, and the nucleic acid construct is inserted into the target genomic locus.

149. A method of expressing a multidomain therapeutic protein comprising a delivery domain fused to a lysosomal alpha-glucosidase protein in a cell in a subject, comprising administering to the subject the composition of any one of claims 1-52, optionally wherein the delivery domain is a CD63-binding delivery domain or a TfR-binding delivery domain,
wherein the coding sequence for the multidomain therapeutic protein is operably linked to a promoter in the nucleic acid construct and is expressed in the cell.

150. A method of expressing a multidomain therapeutic protein comprising a delivery domain fused to a lysosomal alpha-glucosidase protein from a target genomic locus in a cell in a subject, comprising administering to the subject the composition of any one of claims 53-110, optionally wherein the delivery domain is a CD63-binding delivery domain or a TfR-binding delivery domain,
wherein the nuclease agent cleaves the nuclease target site in the target genomic locus, the nucleic acid construct is inserted into the target genomic locus to create a modified target genomic locus, and the multidomain therapeutic protein comprising the delivery domain fused to the lysosomal alpha-glucosidase is expressed from the modified target genomic locus.

151. A method of expressing a multidomain therapeutic protein comprising a CD63-binding delivery domain fused to a lysosomal alpha-glucosidase protein in a cell in a subject, comprising administering to the subject the composition of any one of claims 1-52,
wherein the coding sequence for the multidomain therapeutic protein is operably linked to a promoter in the nucleic acid construct and is expressed in the cell.

152. A method of expressing a multidomain therapeutic protein comprising a CD63-binding delivery domain fused to a lysosomal alpha-glucosidase protein from a target genomic locus in a cell in a subject, comprising administering to the subject the composition of any one of claims 53-110,
wherein the nuclease agent cleaves the nuclease target site in the target genomic locus, the nucleic acid construct is inserted into the target genomic locus to create a modified target genomic locus, and the multidomain therapeutic protein comprising the CD63-binding delivery domain fused to the lysosomal alpha-glucosidase is expressed from the modified target genomic locus.

153. The method of any one of claims 149-152, wherein the expressed multidomain therapeutic protein is delivered to and internalized by skeletal muscle and heart tissue in the subject.

154. The method of any one of claims 147-153, wherein the cell is a liver cell.

155. The method of any one of claims 147-154, wherein the cell is a hepatocyte.

156. The method of any one of claims 147-155, wherein the cell is a human cell.

157. The method of any one of claims 147-156, wherein the cell is a neonatal cell.

158. The method of claim 157, wherein the neonatal subject is a human subject within 24 weeks after birth, optionally wherein the neonatal subject is a human subject within 12 weeks after birth.

159. The method of claim 157, wherein the neonatal subject is a human subject within 8 weeks after birth, optionally wherein the neonatal subject is a human subject within 4 weeks after birth.

160. The method of any one of claims 147-156, wherein the cell is not a neonatal cell.

161. A method of treating a lysosomal alpha-glucosidase deficiency in a subject in need thereof, comprising administering to the subject the composition of any one of claims 1-52,
wherein the coding sequence for the multidomain therapeutic protein is operably linked to a promoter in the nucleic acid construct and is expressed in the subject, optionally wherein the delivery domain is a CD63-binding delivery domain or a TfR-binding delivery domain.

162. A method of treating a lysosomal alpha-glucosidase deficiency in a subject in need thereof, comprising administering to the subject the composition of any one of claims 53-110,
wherein the nuclease agent cleaves the nuclease target site in the target genomic locus, the nucleic acid construct is inserted into the target genomic locus to create a modified target genomic locus, and the multidomain therapeutic protein comprising the delivery domain fused to the lysosomal alpha-glucosidase is expressed from the modified target genomic locus, optionally wherein the delivery domain is a CD63-binding delivery domain or a TfR-binding delivery domain.

163. A method of treating a lysosomal alpha-glucosidase deficiency in a subject in need thereof, comprising administering to the subject the composition of any one of claims 1-52,
wherein the coding sequence for the multidomain therapeutic protein is operably linked to a promoter in the nucleic acid construct and is expressed in the subject.

164. A method of treating a lysosomal alpha-glucosidase deficiency in a subject in need thereof, comprising administering to the subject the composition of any one of claims 53-110,
wherein the nuclease agent cleaves the nuclease target site in the target genomic locus, the nucleic acid construct is inserted into the target genomic locus to create a modified target genomic locus, and the multidomain therapeutic protein comprising the CD63-binding delivery domain fused to the lysosomal alpha-glucosidase is expressed from the modified target genomic locus.

165. A method of reducing glycogen accumulation in a tissue in a subject in need thereof, comprising administering to the subject the composition of any one of claims 1-52,
wherein the coding sequence for the multidomain therapeutic protein is operably linked to a promoter in the nucleic acid construct and is expressed in the subject and reduces glycogen accumulation in the tissue, optionally wherein the delivery domain is a CD63-binding delivery domain or a TfR-binding delivery domain.

166. A method of reducing glycogen accumulation in a tissue in a subject in need thereof, comprising administering to the subject the composition of any one of claims 53-110,
wherein the nuclease agent cleaves the nuclease target site in the target genomic locus, the nucleic acid construct is inserted into the target genomic locus to create a modified target genomic locus, and the multidomain therapeutic protein comprising the delivery domain fused to the lysosomal alpha-glucosidase is expressed from the modified target genomic locus and reduces glycogen accumulation in the tissue, optionally wherein the delivery domain is a CD63-binding delivery domain or a TfR-binding delivery domain.

167. A method of reducing glycogen accumulation in a tissue in a subject in need thereof, comprising administering to the subject the composition of any one of claims 1-52,
wherein the coding sequence for the multidomain therapeutic protein is operably linked to a promoter in the nucleic acid construct and is expressed in the subject and reduces glycogen accumulation in the tissue.

168. A method of reducing glycogen accumulation in a tissue in a subject in need thereof, comprising administering to the subject the composition of any one of claims 53-110,
wherein the nuclease agent cleaves the nuclease target site in the target genomic locus, the nucleic acid construct is inserted into the target genomic locus to create a modified target genomic locus, and the multidomain therapeutic protein comprising the CD63-binding delivery domain fused to the lysosomal alpha-glucosidase is expressed from the modified target genomic locus and reduces glycogen accumulation in the tissue.

169. The method of any one of claims 146-168, wherein the subject has Pompe disease.

170. A method of treating Pompe disease in a subject in need thereof, comprising administering to the subject the composition of any one of claims 1-52,
wherein the coding sequence for the multidomain therapeutic protein is operably linked to a promoter in the nucleic acid construct and is expressed in the subject, thereby treating the Pompe disease, optionally wherein the delivery domain is a CD63-binding delivery domain or a TfR-binding delivery domain.

171. A method of treating Pompe disease in a subject in need thereof, comprising administering to the subject the composition of any one of claims 53-110,
wherein the nuclease agent cleaves the nuclease target site in the target genomic locus, the nucleic acid construct is inserted into the target genomic locus to create a modified target genomic locus, and the multidomain therapeutic protein comprising the delivery domain fused to the lysosomal alpha-glucosidase is expressed from the modified target genomic locus, thereby treating the Pompe disease, optionally wherein the delivery domain is a CD63-binding delivery domain or a TfR-binding delivery domain.

172. A method of treating Pompe disease in a subject in need thereof, comprising administering to the subject the composition of any one of claims 1-52,
wherein the coding sequence for the multidomain therapeutic protein is operably linked to a promoter in the nucleic acid construct and is expressed in the subject, thereby treating the Pompe disease.

173. A method of treating Pompe disease in a subject in need thereof, comprising administering to the subject the composition of any one of claims 53-110,
wherein the nuclease agent cleaves the nuclease target site in the target genomic locus, the nucleic acid construct is inserted into the target genomic locus to create a modified target genomic locus, and the multidomain therapeutic protein comprising the CD63-binding delivery domain fused to the lysosomal alpha-glucosidase is expressed from the modified target genomic locus, thereby treating the Pompe disease.

174. The method of any one of claims 169-173, wherein the Pompe disease is infantile-onset Pompe disease.

175. The method of any one of claims 169-173, wherein the Pompe disease is late-onset Pompe disease.

176. The method of any one of claims 146-175, wherein the subject is a human subject.

177. The method of any one of claims 146-176, wherein the subject is a neonatal subject, optionally wherein the neonatal subject is a human subject within 24 weeks after birth, within 12 weeks after birth, within 8 weeks after birth, or within 4 weeks after birth.

178. The method of any one of claims 146-176, wherein the subject is not a neonatal subject.

179. The method of any one of claims 146-178, wherein the method results in a therapeutically effective level of circulating multidomain therapeutic protein or lysosomal alpha-glucosidase in the subject.

180. The method of any one of claims 146-179, wherein the method reduces glycogen accumulation in skeletal muscle, heart tissue, or central nervous system tissue in the subject, optionally wherein the method reduces glycogen accumulation in skeletal muscle and heart tissue in the subject, and optionally wherein the method results in reduced glycogen levels in skeletal muscle and heart tissue in the subject comparable to wild type levels at the same age.

181. The method of any one of claims 146-180, wherein the method improves muscle strength in the subject or prevents loss of muscle strength in the subject compared to a control subject.

182. The method of claim 181, wherein the method results in the subject having muscle strength comparable to wild type levels at the same age.

183. A method of preventing or reducing the onset of a sign or symptom of Pompe disease in a subject in need thereof, comprising administering to the subject the composition of any one of claims 1-52,
wherein the coding sequence for the multidomain therapeutic protein is operably linked to a promoter in the nucleic acid construct and is expressed in the subject, thereby preventing or reducing the onset of a sign or symptom of the Pompe disease in the subject, optionally wherein the delivery domain is a CD63-binding delivery domain or a TfR-binding delivery domain.

184. A method of preventing or reducing the onset of a sign or symptom of Pompe disease in a subject in need thereof, comprising administering to the subject the composition of any one of claims 53-110,
wherein the nuclease agent cleaves the nuclease target site, the nucleic acid construct is inserted into the target genomic locus to create a modified target genomic locus, and the multidomain therapeutic protein comprising the delivery domain fused to the lysosomal alpha-glucosidase is expressed from the modified target genomic locus, thereby preventing or reducing the onset of a sign or symptom of the Pompe disease in the subject, optionally wherein the delivery domain is a CD63-binding delivery domain or a TfR-binding delivery domain.

185. A method of preventing or reducing the onset of a sign or symptom of Pompe disease in a subject in need thereof, comprising administering to the subject the composition of any one of claims 1-52,
wherein the coding sequence for the multidomain therapeutic protein is operably linked to a promoter in the nucleic acid construct and is expressed in the subject, thereby preventing or reducing the onset of a sign or symptom of the Pompe disease in the subject.

186. A method of preventing or reducing the onset of a sign or symptom of Pompe disease in a subject in need thereof, comprising administering to the subject the composition of any one of claims 53-110,
wherein the nuclease agent cleaves the nuclease target site, the nucleic acid construct is inserted into the target genomic locus to create a modified target genomic locus, and the multidomain therapeutic protein comprising the CD63-binding delivery domain fused to the lysosomal alpha-glucosidase is expressed from the modified target genomic locus, thereby preventing or reducing the onset of a sign or symptom of the Pompe disease in the subject.

187. The method of any one of claims 183-186, wherein the Pompe disease is infantile-onset Pompe disease.

188. The method of any one of claims 183-186, wherein the Pompe disease is late-onset Pompe disease.

189. The method of any one of claims 183-188, wherein the method results in a therapeutically effective level of circulating multidomain therapeutic protein or lysosomal alpha-glucosidase in the subject.

190. The method of any one of claims 183-189, wherein the method prevents or reduces glycogen accumulation in skeletal muscle, heart, or central nervous system tissue in the subject.

191. The method of any one of claims 183-190, wherein the method prevents or reduces glycogen accumulation in skeletal muscle and heart tissue in the subject.

192. The method of any one of claims 183-191, wherein the subject is a human subject.

193. The method of any one of claims 183-192, wherein the subject is a neonatal subject.

194. The method of claim 193, wherein the neonatal subject is a human subject within 24 weeks after birth, optionally wherein the neonatal subject is a human subject within 12 weeks after birth.

195. The method of claim 193, wherein the neonatal subject is a human subject within 8 weeks after birth, optionally wherein the neonatal subject is a human subject within 4 weeks after birth.

196. The method of any one of claims 183-192, wherein the subject is not a neonatal subject.

197. The method of any one of claims 146-196, wherein the method results in increased expression of the multidomain therapeutic protein in the subject compared to a method comprising administering an episomal expression vector encoding the multidomain therapeutic protein to a control subject.

198. The method of any one of claims 146-197, wherein the method results in increased serum levels of the multidomain therapeutic protein in the subject compared to a method comprising administering an episomal expression vector encoding the multidomain therapeutic protein to a control subject.

199. The method of any one of claims 146-198, wherein the method results in serum levels of the multidomain therapeutic protein in the subject of at least about 1 µg/mL, at least about 2 µg/mL, at least about 3 µg/mL, at least about 4 µg/mL, at least about 5 µg/mL, at least about 6 µg/mL, at least about 7 µg/mL, at least about 8 µg/mL, at least about 9 µg/mL, or at least about 10 µg/mL.

200. The method of any one of claims 146-199, wherein the method results in serum levels of the multidomain therapeutic protein in the subject of at least about 2 µg/mL or at least about 5 µg/mL.

201. The method of any one of claims 146-200, wherein the method results in serum levels of the multidomain therapeutic protein in the subject of between about 2 µg/mL and about 30 µg/mL or between about 2 µg/mL and about 20 µg/mL.

202. The method of any one of claims 146-201, wherein the method results in serum levels of the multidomain therapeutic protein in the subject of between about 5 µg/mL and about 30 µg/mL or between about 5 µg/mL and about 20 µg/mL.

203. The method of any one of claims 146-202, wherein the method achieves lysosomal alpha-glucosidase activity levels of at least about 40% of normal, at least about 45% of normal, at least about 50%, at least about 60%, at least about 70%, at least about 80%, at least about 90%, or 100% of normal.

204. The method of any one of claims 146-203, wherein:
(I) the subject has infantile-onset Pompe disease, and the method achieves lysosomal alpha-glucosidase expression or activity levels of at least about 1% or more than about 1% of normal; or
(II) the subject has late-onset Pompe disease, and the method achieves lysosomal alpha-glucosidase expression or activity levels of at least about 40% of normal or more than about 40% of normal.

205. The method of any one of claims 146-204, wherein the expression or activity of the multidomain therapeutic protein is at least 50% of the expression or activity of the multidomain therapeutic protein at a peak level of expression measured for the subject at 24 weeks after the administering.

206. The method of any one of claims 146-205, wherein the expression or activity of the multidomain therapeutic protein is at least 50% of the expression or activity of the multidomain therapeutic protein at a peak level of expression measured for the subject at one year after the administering.

207. The method of any one of claims 146-206, wherein the expression or activity of the multidomain therapeutic protein is at least 60% of the expression or activity of the multidomain therapeutic protein at a peak level of expression measured for the subject at 24 weeks after the administering.

208. The method of any one of claims 146-207, wherein the expression or activity of the multidomain therapeutic protein is at least 50% of the expression or activity of the multidomain therapeutic protein at a peak level of expression measured for the subject at two years after the administering.

209. The method of any one of claims 146-208, wherein the expression or activity of the multidomain therapeutic protein is at least 60% of the expression or activity of the multidomain therapeutic protein at a peak level of expression measured for the subject at 2 years after the administering.

210. The method of any one of claims 146-209, wherein the expression or activity of the multidomain therapeutic protein is at least 60% of the expression or activity of the multidomain therapeutic protein at a peak level of expression measured for the subject at 24 weeks after the administering.

211. The method of any one of claim 146-210, wherein the method further comprises assessing preexisting AAV immunity in the subject prior to administering the nucleic acid construct to the subject.

212. The method of claim 211, wherein the preexisting AAV immunity is preexisting AAV8 immunity.

213. The method of claim 211 or 212, wherein assessing preexisting AAV immunity comprises assessing immunogenicity using a total antibody immune assay or a neutralizing antibody assay.

214. The method of any one of claims 132-213, wherein the nucleic acid construct is administered simultaneously with the nuclease agent or the one or more nucleic acids encoding the nuclease agent.

215. The method of any one of claims 132-213, wherein the nucleic acid construct is not administered simultaneously with the nuclease agent or the one or more nucleic acids encoding the nuclease agent.

216. The method of claim 215, wherein the nucleic acid construct is administered prior to the nuclease agent or the one or more nucleic acids encoding the nuclease agent.

217. The method of claim 215, wherein the nucleic acid construct is administered after the nuclease agent or the one or more nucleic acids encoding the nuclease agent.

218. A composition comprising a nucleic acid construct comprising a coding sequence for a multidomain therapeutic protein comprising a TfR-binding delivery domain fused to a lysosomal alpha-glucosidase, wherein the lysosomal alpha-glucosidase coding sequence is CpG-depleted relative to a wild type lysosomal alpha-glucosidase coding sequence.

219. The composition of claim 218, wherein the TfR-binding delivery domain is fused to the lysosomal alpha-glucosidase protein via a peptide linker.

220. The composition of claim 218 or 219, wherein the lysosomal alpha-glucosidase lacks the lysosomal alpha-glucosidase signal peptide and propeptide.

221. The composition of any one of claims 218-220, wherein the lysosomal alpha-glucosidase comprises the sequence set forth in SEQ ID NO: 173.

222. The composition of any one of claims 218-221, wherein the lysosomal alpha-glucosidase consists of the sequence set forth in SEQ ID NO: 173.

223. The composition of any one of claims 218-222, wherein the lysosomal alpha-glucosidase coding sequence is codon-optimized and CpG-depleted.

224. The composition of any one of claims 218-223, wherein the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and 205-212,
optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176.

225. The composition of any one of claims 218-224, wherein the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and 205-212 and encodes a lysosomal alpha-glucosidase protein comprising SEQ ID NO: 173,
optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176 and encodes a lysosomal alpha-glucosidase protein comprising SEQ ID NO: 173.

226. The composition of any one of claims 218-225, wherein the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and 205-212, is codon-optimized and CpG-depleted, and encodes a lysosomal alpha-glucosidase protein comprising SEQ ID NO: 173,
optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176, is codon-optimized and CpG-depleted, and encodes a lysosomal alpha-glucosidase protein comprising SEQ ID NO: 173.

227. The composition of any one of claims 218-226, wherein the lysosomal alpha-glucosidase coding sequence comprises the sequence set forth in any one of SEQ ID NOS: 174-182 and 205-212,
optionally wherein the lysosomal alpha-glucosidase coding sequence comprises the sequence set forth in SEQ ID NO: 176.

228. The composition of any one of claims 218-227, wherein the lysosomal alpha-glucosidase coding sequence consists of the sequence set forth in any one of SEQ ID NOS: 174-182 and 205-212,
optionally wherein the lysosomal alpha-glucosidase coding sequence consists of the sequence set forth in SEQ ID NO: 176.

229. The composition of any one of claims 218-228, wherein the coding sequence for the TfR-binding delivery domain is codon-optimized or CpG-depleted.

230. The composition of any one of claims 218-229, wherein the coding sequence for the TfR-binding delivery domain is codon-optimized and CpG-depleted.

231. The composition of any one of claims 218-230, wherein the TfR-binding delivery domain comprises an anti-TfR antigen-binding protein.

232. The composition of claim 231, wherein the anti-TfR antigen binding protein comprises:
(i) a HCVR that comprises the HCDR1, HCDR2 and HCDR3 of a HCVR comprising the amino acid sequence set forth in SEQ ID NO: 217, 227, 237, 247, 257, 267, 277, 287, 297, 307, 317, 327, 337, 347, 357, 367, 377, 387, 397, 407, 417, 427, 437, 447, 457, 467, 477, 487, 497, 507, 517, or 527 (or a variant thereof); and/or
(ii) a LCVR that comprises the LCDR1, LCDR2 and LCDR3 of a LCVR comprising the amino acid sequence set forth in SEQ ID NO: 222, 232, 242, 252, 262, 272, 282, 292, 302, 312, 322, 332, 342, 352, 362, 372, 382, 392, 402, 412, 422, 432, 442, 452, 462, 472, 482, 492, 502, 512, 522, or 532 (or a variant thereof).

233. The composition of claim 231 or 232, wherein the anti-TfR antigen binding protein comprises:
(1) a HCVR comprising the HCDR1, HCDR2 and HCDR3 of a HCVR that comprises the amino acid sequence set forth in SEQ ID NO: 217 (or a variant thereof); and a LCVR comprising the LCDR1, LCDR2 and LCDR3 of a LCVR that comprises the amino acid sequence set forth in SEQ ID NO: 222 (or a variant thereof);
(2) a HCVR comprising the HCDR1, HCDR2 and HCDR3 of a HCVR that comprises the amino acid sequence set forth in SEQ ID NO: 227 (or a variant thereof); and a LCVR comprising the LCDR1, LCDR2 and LCDR3 of a LCVR that comprises the amino acid sequence set forth in SEQ ID NO: 232 (or a variant thereof);
(3) a HCVR comprising the HCDR1, HCDR2 and HCDR3 of a HCVR that comprises the amino acid sequence set forth in SEQ ID NO: 237 (or a variant thereof); and a LCVR comprising the LCDR1, LCDR2 and LCDR3 of a LCVR that comprises the amino acid sequence set forth in SEQ ID NO: 242 (or a variant thereof);
(4) a HCVR comprising the HCDR1, HCDR2 and HCDR3 of a HCVR that comprises the amino acid sequence set forth in SEQ ID NO: 247 (or a variant thereof); and a LCVR comprising the LCDR1, LCDR2 and LCDR3 of a LCVR that comprises the amino acid sequence set forth in SEQ ID NO: 252 (or a variant thereof);
(5) a HCVR comprising the HCDR1, HCDR2 and HCDR3 of a HCVR that comprises the amino acid sequence set forth in SEQ ID NO: 257 (or a variant thereof); and a LCVR comprising the LCDR1, LCDR2 and LCDR3 of a LCVR that comprises the amino acid sequence set forth in SEQ ID NO: 262 (or a variant thereof);
(6) a HCVR comprising the HCDR1, HCDR2 and HCDR3 of a HCVR that comprises the amino acid sequence set forth in SEQ ID NO: 267 (or a variant thereof); and a LCVR comprising the LCDR1, LCDR2 and LCDR3 of a LCVR that comprises the amino acid sequence set forth in SEQ ID NO: 272 (or a variant thereof);
(7) a HCVR comprising the HCDR1, HCDR2 and HCDR3 of a HCVR that comprises the amino acid sequence set forth in SEQ ID NO: 277 (or a variant thereof); and a LCVR comprising the LCDR1, LCDR2 and LCDR3 of a LCVR that comprises the amino acid sequence set forth in SEQ ID NO: 282 (or a variant thereof);
(8) a HCVR comprising the HCDR1, HCDR2 and HCDR3 of a HCVR that comprises the amino acid sequence set forth in SEQ ID NO: 287 (or a variant thereof); and a LCVR comprising the LCDR1, LCDR2 and LCDR3 of a LCVR that comprises the amino acid sequence set forth in SEQ ID NO: 292 (or a variant thereof);
(9) a HCVR comprising the HCDR1, HCDR2 and HCDR3 of a HCVR that comprises the amino acid sequence set forth in SEQ ID NO: 297 (or a variant thereof); and a LCVR comprising the LCDR1, LCDR2 and LCDR3 of a LCVR that comprises the amino acid sequence set forth in SEQ ID NO: 302 (or a variant thereof);
(10) a HCVR comprising the HCDR1, HCDR2 and HCDR3 of a HCVR that comprises the amino acid sequence set forth in SEQ ID NO: 307 (or a variant thereof); and a LCVR comprising the LCDR1, LCDR2 and LCDR3 of a LCVR that comprises the amino acid sequence set forth in SEQ ID NO: 312 (or a variant thereof);
(11) a HCVR comprising the HCDR1, HCDR2 and HCDR3 of a HCVR that comprises the amino acid sequence set forth in SEQ ID NO: 317 (or a variant thereof); and a LCVR comprising the LCDR1, LCDR2 and LCDR3 of a LCVR that comprises the amino acid sequence set forth in SEQ ID NO: 322 (or a variant thereof);
(12) a HCVR comprising the HCDR1, HCDR2 and HCDR3 of a HCVR that comprises the amino acid sequence set forth in SEQ ID NO: 327 (or a variant thereof); and a LCVR comprising the LCDR1, LCDR2 and LCDR3 of a LCVR that comprises the amino acid sequence set forth in SEQ ID NO: 332 (or a variant thereof);
(13) a HCVR comprising the HCDR1, HCDR2 and HCDR3 of a HCVR that comprises the amino acid sequence set forth in SEQ ID NO: 337 (or a variant thereof); and a LCVR comprising the LCDR1, LCDR2 and LCDR3 of a LCVR that comprises the amino acid sequence set forth in SEQ ID NO: 342 (or a variant thereof);
(14) a HCVR comprising the HCDR1, HCDR2 and HCDR3 of a HCVR that comprises the amino acid sequence set forth in SEQ ID NO: 347 (or a variant thereof); and a LCVR comprising the LCDR1, LCDR2 and LCDR3 of a LCVR that comprises the amino acid sequence set forth in SEQ ID NO: 352 (or a variant thereof);
(15) a HCVR comprising the HCDR1, HCDR2 and HCDR3 of a HCVR that comprises the amino acid sequence set forth in SEQ ID NO: 357 (or a variant thereof); and a LCVR comprising the LCDR1, LCDR2 and LCDR3 of a LCVR that comprises the amino acid sequence set forth in SEQ ID NO: 362 (or a variant thereof);
(16) a HCVR comprising the HCDR1, HCDR2 and HCDR3 of a HCVR that comprises the amino acid sequence set forth in SEQ ID NO: 367 (or a variant thereof); and a LCVR comprising the LCDR1, LCDR2 and LCDR3 of a LCVR that comprises the amino acid sequence set forth in SEQ ID NO: 372 (or a variant thereof);
(17) a HCVR comprising the HCDR1, HCDR2 and HCDR3 of a HCVR that comprises the amino acid sequence set forth in SEQ ID NO: 377 (or a variant thereof); and a LCVR comprising the LCDR1, LCDR2 and LCDR3 of a LCVR that comprises the amino acid sequence set forth in SEQ ID NO: 382 (or a variant thereof);
(18) a HCVR comprising the HCDR1, HCDR2 and HCDR3 of a HCVR that comprises the amino acid sequence set forth in SEQ ID NO: 387 (or a variant thereof); and a LCVR comprising the LCDR1, LCDR2 and LCDR3 of a LCVR that comprises the amino acid sequence set forth in SEQ ID NO: 392 (or a variant thereof);
(19) a HCVR comprising the HCDR1, HCDR2 and HCDR3 of a HCVR that comprises the amino acid sequence set forth in SEQ ID NO: 397 (or a variant thereof); and a LCVR comprising the LCDR1, LCDR2 and LCDR3 of a LCVR that comprises the amino acid sequence set forth in SEQ ID NO: 402 (or a variant thereof);
(20) a HCVR comprising the HCDR1, HCDR2 and HCDR3 of a HCVR that comprises the amino acid sequence set forth in SEQ ID NO: 407 (or a variant thereof); and a LCVR comprising the LCDR1, LCDR2 and LCDR3 of a LCVR that comprises the amino acid sequence set forth in SEQ ID NO: 412 (or a variant thereof);
(21) a HCVR comprising the HCDR1, HCDR2 and HCDR3 of a HCVR that comprises the amino acid sequence set forth in SEQ ID NO: 417 (or a variant thereof); and a LCVR comprising the LCDR1, LCDR2 and LCDR3 of a LCVR that comprises the amino acid sequence set forth in SEQ ID NO: 422 (or a variant thereof);
(22) a HCVR comprising the HCDR1, HCDR2 and HCDR3 of a HCVR that comprises the amino acid sequence set forth in SEQ ID NO: 427 (or a variant thereof); and a LCVR comprising the LCDR1, LCDR2 and LCDR3 of a LCVR that comprises the amino acid sequence set forth in SEQ ID NO: 432 (or a variant thereof);
(23) a HCVR comprising the HCDR1, HCDR2 and HCDR3 of a HCVR that comprises the amino acid sequence set forth in SEQ ID NO: 437 (or a variant thereof); and a LCVR comprising the LCDR1, LCDR2 and LCDR3 of a LCVR that comprises the amino acid sequence set forth in SEQ ID NO: 442 (or a variant thereof);
(24) a HCVR comprising the HCDR1, HCDR2 and HCDR3 of a HCVR that comprises the amino acid sequence set forth in SEQ ID NO: 447 (or a variant thereof); and a LCVR comprising the LCDR1, LCDR2 and LCDR3 of a LCVR that comprises the amino acid sequence set forth in SEQ ID NO: 452 (or a variant thereof);
(25) a HCVR comprising the HCDR1, HCDR2 and HCDR3 of a HCVR that comprises the amino acid sequence set forth in SEQ ID NO: 457 (or a variant thereof); and a LCVR comprising the LCDR1, LCDR2 and LCDR3 of a LCVR that comprises the amino acid sequence set forth in SEQ ID NO: 462 (or a variant thereof);
(26) a HCVR comprising the HCDR1, HCDR2 and HCDR3 of a HCVR that comprises the amino acid sequence set forth in SEQ ID NO: 467 (or a variant thereof); and a LCVR comprising the LCDR1, LCDR2 and LCDR3 of a LCVR that comprises the amino acid sequence set forth in SEQ ID NO: 472 (or a variant thereof);
(27) a HCVR comprising the HCDR1, HCDR2 and HCDR3 of a HCVR that comprises the amino acid sequence set forth in SEQ ID NO: 477 (or a variant thereof); and a LCVR comprising the LCDR1, LCDR2 and LCDR3 of a LCVR that comprises the amino acid sequence set forth in SEQ ID NO: 482 (or a variant thereof);
(28) a HCVR comprising the HCDR1, HCDR2 and HCDR3 of a HCVR that comprises the amino acid sequence set forth in SEQ ID NO: 487 (or a variant thereof); and a LCVR comprising the LCDR1, LCDR2 and LCDR3 of a LCVR that comprises the amino acid sequence set forth in SEQ ID NO: 492 (or a variant thereof);
(29) a HCVR comprising the HCDR1, HCDR2 and HCDR3 of a HCVR that comprises the amino acid sequence set forth in SEQ ID NO: 497 (or a variant thereof); and a LCVR comprising the LCDR1, LCDR2 and LCDR3 of a LCVR that comprises the amino acid sequence set forth in SEQ ID NO: 502 (or a variant thereof);
(30) a HCVR comprising the HCDR1, HCDR2 and HCDR3 of a HCVR that comprises the amino acid sequence set forth in SEQ ID NO: 507 (or a variant thereof); and a LCVR comprising the LCDR1, LCDR2 and LCDR3 of a LCVR that comprises the amino acid sequence set forth in SEQ ID NO: 512 (or a variant thereof);
(31) a HCVR comprising the HCDR1, HCDR2 and HCDR3 of a HCVR that comprises the amino acid sequence set forth in SEQ ID NO: 517 (or a variant thereof); and a LCVR comprising the LCDR1, LCDR2 and LCDR3 of a LCVR that comprises the amino acid sequence set forth in SEQ ID NO: 522 (or a variant thereof); or
(32) a HCVR comprising the HCDR1, HCDR2 and HCDR3 of a HCVR that comprises the amino acid sequence set forth in SEQ ID NO: 527 (or a variant thereof); and a LCVR comprising the LCDR1, LCDR2 and LCDR3 of a LCVR that comprises the amino acid sequence set forth in SEQ ID NO: 532 (or a variant thereof).

234. The composition of any one of claims 231-233, wherein the anti-TfR antigen binding protein comprises:
(1) a HCVR comprising the HCDR1, HCDR2 and HCDR3 of a HCVR that comprises the amino acid sequence set forth in SEQ ID NO: 437 (or a variant thereof); and a LCVR comprising the LCDR1, LCDR2 and LCDR3 of a LCVR that comprises the amino acid sequence set forth in SEQ ID NO: 442 (or a variant thereof); or
(2) a HCVR comprising the HCDR1, HCDR2 and HCDR3 of a HCVR that comprises the amino acid sequence set forth in SEQ ID NO: 457 (or a variant thereof); and a LCVR comprising the LCDR1, LCDR2 and LCDR3 of a LCVR that comprises the amino acid sequence set forth in SEQ ID NO: 462 (or a variant thereof).

235. The composition of any one of claims 231-234, wherein the anti-TfR antigen binding protein comprises:
(a) a HCVR that comprises: an HCDR1 comprising the amino acid sequence set forth in SEQ ID NO: 218 (or a variant thereof), an HCDR2 comprising the amino acid sequence set forth in SEQ ID NO: 219 (or a variant thereof), and an HCDR3 comprising the amino acid sequence set forth in SEQ ID NO: 220 (or a variant thereof); and a LCVR that comprises: an LCDR1 comprising the amino acid sequence set forth in SEQ ID NO: 223 (or a variant thereof), an LCDR2 comprising the amino acid sequence set forth in SEQ ID NO: 224 (or a variant thereof), and an LCDR3 comprising the amino acid sequence set forth in SEQ ID NO: 225 (or a variant thereof);
(b) a HCVR that comprises: an HCDR1 comprising the amino acid sequence set forth in SEQ ID NO: 228 (or a variant thereof), an HCDR2 comprising the amino acid sequence set forth in SEQ ID NO: 229 (or a variant thereof), and an HCDR3 comprising the amino acid sequence set forth in SEQ ID NO: 230 (or a variant thereof); and a LCVR that comprises: an LCDR1 comprising the amino acid sequence set forth in SEQ ID NO: 233 (or a variant thereof), an LCDR2 comprising the amino acid sequence set forth in SEQ ID NO: 234 (or a variant thereof), and an LCDR3 comprising the amino acid sequence set forth in SEQ ID NO: 235 (or a variant thereof);
(c) a HCVR that comprises: an HCDR1 comprising the amino acid sequence set forth in SEQ ID NO: 238 (or a variant thereof), an HCDR2 comprising the amino acid sequence set forth in SEQ ID NO: 239 (or a variant thereof), and an HCDR3 comprising the amino acid sequence set forth in SEQ ID NO: 240 (or a variant thereof); and a LCVR that comprises: an LCDR1 comprising the amino acid sequence set forth in SEQ ID NO: 243 (or a variant thereof), an LCDR2 comprising the amino acid sequence set forth in SEQ ID NO: 244 (or a variant thereof), and an LCDR3 comprising the amino acid sequence set forth in SEQ ID NO: 245 (or a variant thereof);
(d) a HCVR that comprises: an HCDR1 comprising the amino acid sequence set forth in SEQ ID NO: 248 (or a variant thereof), an HCDR2 comprising the amino acid sequence set forth in SEQ ID NO: 249 (or a variant thereof), and an HCDR3 comprising the amino acid sequence set forth in SEQ ID NO: 250 (or a variant thereof); and a LCVR that comprises: an LCDR1 comprising the amino acid sequence set forth in SEQ ID NO: 253 (or a variant thereof), an LCDR2 comprising the amino acid sequence set forth in SEQ ID NO: 254 (or a variant thereof), and an LCDR3 comprising the amino acid sequence set forth in SEQ ID NO: 255 (or a variant thereof);
(e) a HCVR that comprises: an HCDR1 comprising the amino acid sequence set forth in SEQ ID NO: 258 (or a variant thereof), an HCDR2 comprising the amino acid sequence set forth in SEQ ID NO: 259 (or a variant thereof), and an HCDR3 comprising the amino acid sequence set forth in SEQ ID NO: 260 (or a variant thereof); and a LCVR that comprises: an LCDR1 comprising the amino acid sequence set forth in SEQ ID NO: 263 (or a variant thereof), an LCDR2 comprising the amino acid sequence set forth in SEQ ID NO: 264 (or a variant thereof), and an LCDR3 comprising the amino acid sequence set forth in SEQ ID NO: 265 (or a variant thereof);
(f) a HCVR that comprises: an HCDR1 comprising the amino acid sequence set forth in SEQ ID NO: 268 (or a variant thereof), an HCDR2 comprising the amino acid sequence set forth in SEQ ID NO: 269 (or a variant thereof), and an HCDR3 comprising the amino acid sequence set forth in SEQ ID NO: 270 (or a variant thereof); and a LCVR that comprises: an LCDR1 comprising the amino acid sequence set forth in SEQ ID NO: 273 (or a variant thereof), an LCDR2 comprising the amino acid sequence set forth in SEQ ID NO: 274 (or a variant thereof), and an LCDR3 comprising the amino acid sequence set forth in SEQ ID NO: 275 (or a variant thereof);
(g) a HCVR that comprises: an HCDR1 comprising the amino acid sequence set forth in SEQ ID NO: 278 (or a variant thereof), an HCDR2 comprising the amino acid sequence set forth in SEQ ID NO: 279 (or a variant thereof), and an HCDR3 comprising the amino acid sequence set forth in SEQ ID NO: 280 (or a variant thereof); and a LCVR that comprises: an LCDR1 comprising the amino acid sequence set forth in SEQ ID NO: 283 (or a variant thereof), an LCDR2 comprising the amino acid sequence set forth in SEQ ID NO: 284 (or a variant thereof), and an LCDR3 comprising the amino acid sequence set forth in SEQ ID NO: 285 (or a variant thereof);
(h) a HCVR that comprises: an HCDR1 comprising the amino acid sequence set forth in SEQ ID NO: 288 (or a variant thereof), an HCDR2 comprising the amino acid sequence set forth in SEQ ID NO: 289 (or a variant thereof), and an HCDR3 comprising the amino acid sequence set forth in SEQ ID NO: 290 (or a variant thereof); and a LCVR that comprises: an LCDR1 comprising the amino acid sequence set forth in SEQ ID NO: 293 (or a variant thereof), an LCDR2 comprising the amino acid sequence set forth in SEQ ID NO: 294 (or a variant thereof), and an LCDR3 comprising the amino acid sequence set forth in SEQ ID NO: 295 (or a variant thereof);
(i) a HCVR that comprises: an HCDR1 comprising the amino acid sequence set forth in SEQ ID NO: 298 (or a variant thereof), an HCDR2 comprising the amino acid sequence set forth in SEQ ID NO: 299 (or a variant thereof), and an HCDR3 comprising the amino acid sequence set forth in SEQ ID NO: 300 (or a variant thereof); and a LCVR that comprises: an LCDR1 comprising the amino acid sequence set forth in SEQ ID NO: 303 (or a variant thereof), an LCDR2 comprising the amino acid sequence set forth in SEQ ID NO: 304 (or a variant thereof), and an LCDR3 comprising the amino acid sequence set forth in SEQ ID NO: 305 (or a variant thereof);
(j) a HCVR that comprises: an HCDR1 comprising the amino acid sequence set forth in SEQ ID NO: 308 (or a variant thereof), an HCDR2 comprising the amino acid sequence set forth in SEQ ID NO: 309 (or a variant thereof), and an HCDR3 comprising the amino acid sequence set forth in SEQ ID NO: 310 (or a variant thereof); and a LCVR that comprises: an LCDR1 comprising the amino acid sequence set forth in SEQ ID NO: 313 (or a variant thereof), an LCDR2 comprising the amino acid sequence set forth in SEQ ID NO: 314 (or a variant thereof), and an LCDR3 comprising the amino acid sequence set forth in SEQ ID NO: 315 (or a variant thereof);
(k) a HCVR that comprises: an HCDR1 comprising the amino acid sequence set forth in SEQ ID NO: 318 (or a variant thereof), an HCDR2 comprising the amino acid sequence set forth in SEQ ID NO: 319 (or a variant thereof), and an HCDR3 comprising the amino acid sequence set forth in SEQ ID NO: 320 (or a variant thereof); and a LCVR that comprises: an LCDR1 comprising the amino acid sequence set forth in SEQ ID NO: 323 (or a variant thereof), an LCDR2 comprising the amino acid sequence set forth in SEQ ID NO: 324 (or a variant thereof), and an LCDR3 comprising the amino acid sequence set forth in SEQ ID NO: 325 (or a variant thereof);
(1) a HCVR that comprises: an HCDR1 comprising the amino acid sequence set forth in SEQ ID NO: 328 (or a variant thereof), an HCDR2 comprising the amino acid sequence set forth in SEQ ID NO: 329 (or a variant thereof), and an HCDR3 comprising the amino acid sequence set forth in SEQ ID NO: 330 (or a variant thereof); and a LCVR that comprises: an LCDR1 comprising the amino acid sequence set forth in SEQ ID NO: 333 (or a variant thereof), an LCDR2 comprising the amino acid sequence set forth in SEQ ID NO: 334 (or a variant thereof), and an LCDR3 comprising the amino acid sequence set forth in SEQ ID NO: 335 (or a variant thereof);
(m) a HCVR that comprises: an HCDR1 comprising the amino acid sequence set forth in SEQ ID NO: 338 (or a variant thereof), an HCDR2 comprising the amino acid sequence set forth in SEQ ID NO: 339 (or a variant thereof), and an HCDR3 comprising the amino acid sequence set forth in SEQ ID NO: 340 (or a variant thereof); and a LCVR that comprises: an LCDR1 comprising the amino acid sequence set forth in SEQ ID NO: 343 (or a variant thereof), an LCDR2 comprising the amino acid sequence set forth in SEQ ID NO: 344 (or a variant thereof), and an LCDR3 comprising the amino acid sequence set forth in SEQ ID NO: 345 (or a variant thereof);
(n) a HCVR that comprises: an HCDR1 comprising the amino acid sequence set forth in SEQ ID NO: 348 (or a variant thereof), an HCDR2 comprising the amino acid sequence set forth in SEQ ID NO: 349 (or a variant thereof), and an HCDR3 comprising the amino acid sequence set forth in SEQ ID NO: 350 (or a variant thereof); and a LCVR that comprises: an LCDR1 comprising the amino acid sequence set forth in SEQ ID NO: 353 (or a variant thereof), an LCDR2 comprising the amino acid sequence set forth in SEQ ID NO: 354 (or a variant thereof), and an LCDR3 comprising the amino acid sequence set forth in SEQ ID NO: 355 (or a variant thereof);
(o) a HCVR that comprises: an HCDR1 comprising the amino acid sequence set forth in SEQ ID NO: 358 (or a variant thereof), an HCDR2 comprising the amino acid sequence set forth in SEQ ID NO: 359 (or a variant thereof), and an HCDR3 comprising the amino acid sequence set forth in SEQ ID NO: 360 (or a variant thereof); and a LCVR that comprises: an LCDR1 comprising the amino acid sequence set forth in SEQ ID NO: 363 (or a variant thereof), an LCDR2 comprising the amino acid sequence set forth in SEQ ID NO: 364 (or a variant thereof), and an LCDR3 comprising the amino acid sequence set forth in SEQ ID NO: 365 (or a variant thereof);
(p) a HCVR that comprises: an HCDR1 comprising the amino acid sequence set forth in SEQ ID NO: 368 (or a variant thereof), an HCDR2 comprising the amino acid sequence set forth in SEQ ID NO: 369 (or a variant thereof), and an HCDR3 comprising the amino acid sequence set forth in SEQ ID NO: 370 (or a variant thereof); and a LCVR that comprises: an LCDR1 comprising the amino acid sequence set forth in SEQ ID NO: 373 (or a variant thereof), an LCDR2 comprising the amino acid sequence set forth in SEQ ID NO: 374 (or a variant thereof), and an LCDR3 comprising the amino acid sequence set forth in SEQ ID NO: 375 (or a variant thereof);
(q) a HCVR that comprises: an HCDR1 comprising the amino acid sequence set forth in SEQ ID NO: 378 (or a variant thereof), an HCDR2 comprising the amino acid sequence set forth in SEQ ID NO: 379 (or a variant thereof), and an HCDR3 comprising the amino acid sequence set forth in SEQ ID NO: 380 (or a variant thereof); and a LCVR that comprises: an LCDR1 comprising the amino acid sequence set forth in SEQ ID NO: 383 (or a variant thereof), an LCDR2 comprising the amino acid sequence set forth in SEQ ID NO: 384 (or a variant thereof), and an LCDR3 comprising the amino acid sequence set forth in SEQ ID NO: 385 (or a variant thereof);
(r) a HCVR that comprises: an HCDR1 comprising the amino acid sequence set forth in SEQ ID NO: 388 (or a variant thereof), an HCDR2 comprising the amino acid sequence set forth in SEQ ID NO: 389 (or a variant thereof), and an HCDR3 comprising the amino acid sequence set forth in SEQ ID NO: 390 (or a variant thereof); and a LCVR that comprises: an LCDR1 comprising the amino acid sequence set forth in SEQ ID NO: 393 (or a variant thereof), an LCDR2 comprising the amino acid sequence set forth in SEQ ID NO: 394 (or a variant thereof), and an LCDR3 comprising the amino acid sequence set forth in SEQ ID NO: 395 (or a variant thereof);
(s) a HCVR that comprises: an HCDR1 comprising the amino acid sequence set forth in SEQ ID NO: 398 (or a variant thereof), an HCDR2 comprising the amino acid sequence set forth in SEQ ID NO: 399 (or a variant thereof), and an HCDR3 comprising the amino acid sequence set forth in SEQ ID NO: 400 (or a variant thereof); and a LCVR that comprises: an LCDR1 comprising the amino acid sequence set forth in SEQ ID NO: 403 (or a variant thereof), an LCDR2 comprising the amino acid sequence set forth in SEQ ID NO: 404 (or a variant thereof), and an LCDR3 comprising the amino acid sequence set forth in SEQ ID NO: 405 (or a variant thereof);
(t) a HCVR that comprises: an HCDR1 comprising the amino acid sequence set forth in SEQ ID NO: 408 (or a variant thereof), an HCDR2 comprising the amino acid sequence set forth in SEQ ID NO: 409 (or a variant thereof), and an HCDR3 comprising the amino acid sequence set forth in SEQ ID NO: 410 (or a variant thereof); and a LCVR that comprises: an LCDR1 comprising the amino acid sequence set forth in SEQ ID NO: 413 (or a variant thereof), an LCDR2 comprising the amino acid sequence set forth in SEQ ID NO: 414 (or a variant thereof), and an LCDR3 comprising the amino acid sequence set forth in SEQ ID NO: 415 (or a variant thereof);
(u) a HCVR that comprises: an HCDR1 comprising the amino acid sequence set forth in SEQ ID NO: 418 (or a variant thereof), an HCDR2 comprising the amino acid sequence set forth in SEQ ID NO: 419 (or a variant thereof), and an HCDR3 comprising the amino acid sequence set forth in SEQ ID NO: 420 (or a variant thereof); and a LCVR that comprises: an LCDR1 comprising the amino acid sequence set forth in SEQ ID NO: 423 (or a variant thereof), an LCDR2 comprising the amino acid sequence set forth in SEQ ID NO: 424 (or a variant thereof), and an LCDR3 comprising the amino acid sequence set forth in SEQ ID NO: 425 (or a variant thereof);
(v) a HCVR that comprises: an HCDR1 comprising the amino acid sequence set forth in SEQ ID NO: 428 (or a variant thereof), an HCDR2 comprising the amino acid sequence set forth in SEQ ID NO: 429 (or a variant thereof), and an HCDR3 comprising the amino acid sequence set forth in SEQ ID NO: 430 (or a variant thereof); and a LCVR that comprises: an LCDR1 comprising the amino acid sequence set forth in SEQ ID NO: 433 (or a variant thereof), an LCDR2 comprising the amino acid sequence set forth in SEQ ID NO: 434 (or a variant thereof), and an LCDR3 comprising the amino acid sequence set forth in SEQ ID NO: 435 (or a variant thereof);
(w) a HCVR that comprises: an HCDR1 comprising the amino acid sequence set forth in SEQ ID NO: 438 (or a variant thereof), an HCDR2 comprising the amino acid sequence set forth in SEQ ID NO: 439 (or a variant thereof), and an HCDR3 comprising the amino acid sequence set forth in SEQ ID NO: 440 (or a variant thereof); and a LCVR that comprises: an LCDR1 comprising the amino acid sequence set forth in SEQ ID NO: 443 (or a variant thereof), an LCDR2 comprising the amino acid sequence set forth in SEQ ID NO: 444 (or a variant thereof), and an LCDR3 comprising the amino acid sequence set forth in SEQ ID NO: 445 (or a variant thereof);
(x) a HCVR that comprises: an HCDR1 comprising the amino acid sequence set forth in SEQ ID NO: 448 (or a variant thereof), an HCDR2 comprising the amino acid sequence set forth in SEQ ID NO: 449 (or a variant thereof), and an HCDR3 comprising the amino acid sequence set forth in SEQ ID NO: 450 (or a variant thereof); and a LCVR that comprises: an LCDR1 comprising the amino acid sequence set forth in SEQ ID NO: 453 (or a variant thereof), an LCDR2 comprising the amino acid sequence set forth in SEQ ID NO: 454 (or a variant thereof), and an LCDR3 comprising the amino acid sequence set forth in SEQ ID NO: 455 (or a variant thereof);
(y) a HCVR that comprises: an HCDR1 comprising the amino acid sequence set forth in SEQ ID NO: 458 (or a variant thereof), an HCDR2 comprising the amino acid sequence set forth in SEQ ID NO: 459 (or a variant thereof), and an HCDR3 comprising the amino acid sequence set forth in SEQ ID NO: 460 (or a variant thereof); and a LCVR that comprises: an LCDR1 comprising the amino acid sequence set forth in SEQ ID NO: 463 (or a variant thereof), an LCDR2 comprising the amino acid sequence set forth in SEQ ID NO: 464 (or a variant thereof), and an LCDR3 comprising the amino acid sequence set forth in SEQ ID NO: 465 (or a variant thereof);
(z) a HCVR that comprises: an HCDR1 comprising the amino acid sequence set forth in SEQ ID NO: 468 (or a variant thereof), an HCDR2 comprising the amino acid sequence set forth in SEQ ID NO: 469 (or a variant thereof), and an HCDR3 comprising the amino acid sequence set forth in SEQ ID NO: 470 (or a variant thereof); and a LCVR that comprises: an LCDR1 comprising the amino acid sequence set forth in SEQ ID NO: 473 (or a variant thereof), an LCDR2 comprising the amino acid sequence set forth in SEQ ID NO: 474 (or a variant thereof), and an LCDR3 comprising the amino acid sequence set forth in SEQ ID NO: 475 (or a variant thereof);
(aa) a HCVR that comprises: an HCDR1 comprising the amino acid sequence set forth in SEQ ID NO: 478 (or a variant thereof), an HCDR2 comprising the amino acid sequence set forth in SEQ ID NO: 479 (or a variant thereof), and an HCDR3 comprising the amino acid sequence set forth in SEQ ID NO: 480 (or a variant thereof); and a LCVR that comprises: an LCDR1 comprising the amino acid sequence set forth in SEQ ID NO: 483 (or a variant thereof), an LCDR2 comprising the amino acid sequence set forth in SEQ ID NO: 484 (or a variant thereof), and an LCDR3 comprising the amino acid sequence set forth in SEQ ID NO: 485 (or a variant thereof);
(ab) a HCVR that comprises: an HCDR1 comprising the amino acid sequence set forth in SEQ ID NO: 488 (or a variant thereof), an HCDR2 comprising the amino acid sequence set forth in SEQ ID NO: 489 (or a variant thereof), and an HCDR3 comprising the amino acid sequence set forth in SEQ ID NO: 490 (or a variant thereof); and a LCVR that comprises: an LCDR1 comprising the amino acid sequence set forth in SEQ ID NO: 493 (or a variant thereof), an LCDR2 comprising the amino acid sequence set forth in SEQ ID NO: 494 (or a variant thereof), and an LCDR3 comprising the amino acid sequence set forth in SEQ ID NO: 495 (or a variant thereof);
(ac) a HCVR that comprises: an HCDR1 comprising the amino acid sequence set forth in SEQ ID NO: 498 (or a variant thereof), an HCDR2 comprising the amino acid sequence set forth in SEQ ID NO: 499 (or a variant thereof), and an HCDR3 comprising the amino acid sequence set forth in SEQ ID NO: 500 (or a variant thereof); and a LCVR that comprises: an LCDR1 comprising the amino acid sequence set forth in SEQ ID NO: 503 (or a variant thereof), an LCDR2 comprising the amino acid sequence set forth in SEQ ID NO: 504 (or a variant thereof), and an LCDR3 comprising the amino acid sequence set forth in SEQ ID NO: 505 (or a variant thereof);
(ad) a HCVR that comprises: an HCDR1 comprising the amino acid sequence set forth in SEQ ID NO: 508 (or a variant thereof), an HCDR2 comprising the amino acid sequence set forth in SEQ ID NO: 509 (or a variant thereof), and an HCDR3 comprising the amino acid sequence set forth in SEQ ID NO: 510 (or a variant thereof); and a LCVR that comprises: an LCDR1 comprising the amino acid sequence set forth in SEQ ID NO: 513 (or a variant thereof), an LCDR2 comprising the amino acid sequence set forth in SEQ ID NO: 514 (or a variant thereof), and an LCDR3 comprising the amino acid sequence set forth in SEQ ID NO: 515 (or a variant thereof);
(ae) a HCVR that comprises: an HCDR1 comprising the amino acid sequence set forth in SEQ ID NO: 518 (or a variant thereof), an HCDR2 comprising the amino acid sequence set forth in SEQ ID NO: 519 (or a variant thereof), and an HCDR3 comprising the amino acid sequence set forth in SEQ ID NO: 520 (or a variant thereof); and a LCVR that comprises: an LCDR1 comprising the amino acid sequence set forth in SEQ ID NO: 523 (or a variant thereof), an LCDR2 comprising the amino acid sequence set forth in SEQ ID NO: 524 (or a variant thereof), and an LCDR3 comprising the amino acid sequence set forth in SEQ ID NO: 525 (or a variant thereof); and/or
(af) a HCVR that comprises: an HCDR1 comprising the amino acid sequence set forth in SEQ ID NO: 528 (or a variant thereof), an HCDR2 comprising the amino acid sequence set forth in SEQ ID NO: 529 (or a variant thereof), and an HCDR3 comprising the amino acid sequence set forth in SEQ ID NO: 530 (or a variant thereof); and a LCVR that comprises: an LCDR1 comprising the amino acid sequence set forth in SEQ ID NO: 533 (or a variant thereof), an LCDR2 comprising the amino acid sequence set forth in SEQ ID NO: 534 (or a variant thereof), and an LCDR3 comprising the amino acid sequence set forth in SEQ ID NO: 535 (or a variant thereof).

236. The composition of any one of claims 231-235, wherein the anti-TfR antigen binding protein comprises:
(a) a HCVR that comprises: an HCDR1 comprising the amino acid sequence set forth in SEQ ID NO: 438 (or a variant thereof), an HCDR2 comprising the amino acid sequence set forth in SEQ ID NO: 439 (or a variant thereof), and an HCDR3 comprising the amino acid sequence set forth in SEQ ID NO: 440 (or a variant thereof); and a LCVR that comprises: an LCDR1 comprising the amino acid sequence set forth in SEQ ID NO: 443 (or a variant thereof), an LCDR2 comprising the amino acid sequence set forth in SEQ ID NO: 444 (or a variant thereof), and an LCDR3 comprising the amino acid sequence set forth in SEQ ID NO: 445 (or a variant thereof); or
(b) a HCVR that comprises: an HCDR1 comprising the amino acid sequence set forth in SEQ ID NO: 458 (or a variant thereof), an HCDR2 comprising the amino acid sequence set forth in SEQ ID NO: 459 (or a variant thereof), and an HCDR3 comprising the amino acid sequence set forth in SEQ ID NO: 460 (or a variant thereof); and a LCVR that comprises: an LCDR1 comprising the amino acid sequence set forth in SEQ ID NO: 463 (or a variant thereof), an LCDR2 comprising the amino acid sequence set forth in SEQ ID NO: 464 (or a variant thereof), and an LCDR3 comprising the amino acid sequence set forth in SEQ ID NO: 465 (or a variant thereof).

237. The composition of any one of claims 231-236, wherein the anti-TfR antigen binding protein comprises:
(i) a HCVR that comprises the amino acid sequence set forth in SEQ ID NO: 217 (or a variant thereof); and a LCVR that comprises the amino acid sequence set forth in SEQ ID NO: 222 (or a variant thereof);
(ii) a HCVR that comprises the amino acid sequence set forth in SEQ ID NO: 227 (or a variant thereof); and a LCVR that comprises the amino acid sequence set forth in SEQ ID NO: 232 (or a variant thereof);
(iii) a HCVR that comprises the amino acid sequence set forth in SEQ ID NO: 237 (or a variant thereof); and a LCVR that comprises the amino acid sequence set forth in SEQ ID NO: 242 (or a variant thereof);
(iv) a HCVR that comprises the amino acid sequence set forth in SEQ ID NO: 247 (or a variant thereof); and a LCVR that comprises the amino acid sequence set forth in SEQ ID NO: 252 (or a variant thereof);
(v) a HCVR that comprises the amino acid sequence set forth in SEQ ID NO: 257 (or a variant thereof); and a LCVR that comprises the amino acid sequence set forth in SEQ ID NO: 262 (or a variant thereof);
(vi) a HCVR that comprises the amino acid sequence set forth in SEQ ID NO: 267 (or a variant thereof); and a LCVR that comprises the amino acid sequence set forth in SEQ ID NO: 272 (or a variant thereof);
(vii) a HCVR that comprises the amino acid sequence set forth in SEQ ID NO: 277 (or a variant thereof); and a LCVR that comprises the amino acid sequence set forth in SEQ ID NO: 282 (or a variant thereof);
(viii) a HCVR that comprises the amino acid sequence set forth in SEQ ID NO: 287 (or a variant thereof); and a LCVR that comprises the amino acid sequence set forth in SEQ ID NO: 292 (or a variant thereof);
(ix) a HCVR that comprises the amino acid sequence set forth in SEQ ID NO: 297 (or a variant thereof); and a LCVR that comprises the amino acid sequence set forth in SEQ ID NO: 302 (or a variant thereof);
(x) a HCVR that comprises the amino acid sequence set forth in SEQ ID NO: 307 (or a variant thereof); and a LCVR that comprises the amino acid sequence set forth in SEQ ID NO: 312 (or a variant thereof);
(xi) a HCVR that comprises the amino acid sequence set forth in SEQ ID NO: 317 (or a variant thereof); and a LCVR that comprises the amino acid sequence set forth in SEQ ID NO: 322 (or a variant thereof);
(xii) a HCVR that comprises the amino acid sequence set forth in SEQ ID NO: 327 (or a variant thereof); and a LCVR that comprises the amino acid sequence set forth in SEQ ID NO: 332 (or a variant thereof);
(xiii) a HCVR that comprises the amino acid sequence set forth in SEQ ID NO: 337 (or a variant thereof); and a LCVR that comprises the amino acid sequence set forth in SEQ ID NO: 342 (or a variant thereof);
(xiv) a HCVR that comprises the amino acid sequence set forth in SEQ ID NO: 347 (or a variant thereof); and a LCVR that comprises the amino acid sequence set forth in SEQ ID NO: 352 (or a variant thereof);
(xv) a HCVR that comprises the amino acid sequence set forth in SEQ ID NO: 357 (or a variant thereof); and a LCVR that comprises the amino acid sequence set forth in SEQ ID NO: 362 (or a variant thereof);
(xvi) a HCVR that comprises the amino acid sequence set forth in SEQ ID NO: 367 (or a variant thereof); and a LCVR that comprises the amino acid sequence set forth in SEQ ID NO: 372 (or a variant thereof);
(xvii) a HCVR that comprises the amino acid sequence set forth in SEQ ID NO: 377 (or a variant thereof); and a LCVR that comprises the amino acid sequence set forth in SEQ ID NO: 382 (or a variant thereof);
(xviii) a HCVR that comprises the amino acid sequence set forth in SEQ ID NO: 387 (or a variant thereof); and a LCVR that comprises the amino acid sequence set forth in SEQ ID NO: 392 (or a variant thereof);
(xix) a HCVR that comprises the amino acid sequence set forth in SEQ ID NO: 397 (or a variant thereof); and a LCVR that comprises the amino acid sequence set forth in SEQ ID NO: 402 (or a variant thereof);
(xx) a HCVR that comprises the amino acid sequence set forth in SEQ ID NO: 407 (or a variant thereof); and a LCVR that comprises the amino acid sequence set forth in SEQ ID NO: 412 (or a variant thereof);
(xxi) a HCVR that comprises the amino acid sequence set forth in SEQ ID NO: 417 (or a variant thereof); and a LCVR that comprises the amino acid sequence set forth in SEQ ID NO: 422 (or a variant thereof);
(xxii) a HCVR that comprises the amino acid sequence set forth in SEQ ID NO: 427 (or a variant thereof); and a LCVR that comprises the amino acid sequence set forth in SEQ ID NO: 432 (or a variant thereof);
(xxiii) a HCVR that comprises the amino acid sequence set forth in SEQ ID NO: 437 (or a variant thereof); and a LCVR that comprises the amino acid sequence set forth in SEQ ID NO: 442 (or a variant thereof);
(xxiv) a HCVR that comprises the amino acid sequence set forth in SEQ ID NO: 447 (or a variant thereof); and a LCVR that comprises the amino acid sequence set forth in SEQ ID NO: 452 (or a variant thereof);
(xxv) a HCVR that comprises the amino acid sequence set forth in SEQ ID NO: 457 (or a variant thereof); and a LCVR that comprises the amino acid sequence set forth in SEQ ID NO: 462 (or a variant thereof);
(xxvi) a HCVR that comprises the amino acid sequence set forth in SEQ ID NO: 467 (or a variant thereof); and a LCVR that comprises the amino acid sequence set forth in SEQ ID NO: 472 (or a variant thereof);
(xxvii) a HCVR that comprises the amino acid sequence set forth in SEQ ID NO: 477 (or a variant thereof); and a LCVR that comprises the amino acid sequence set forth in SEQ ID NO: 482 (or a variant thereof);
(xxviii) a HCVR that comprises the amino acid sequence set forth in SEQ ID NO: 487 (or a variant thereof); and a LCVR that comprises the amino acid sequence set forth in SEQ ID NO: 492 (or a variant thereof);
(xxix) a HCVR that comprises the amino acid sequence set forth in SEQ ID NO: 497 (or a variant thereof); and a LCVR that comprises the amino acid sequence set forth in SEQ ID NO: 502 (or a variant thereof);
(xxx) a HCVR that comprises the amino acid sequence set forth in SEQ ID NO: 507 (or a variant thereof); and a LCVR that comprises the amino acid sequence set forth in SEQ ID NO: 512 (or a variant thereof);
(xxxi) a HCVR that comprises the amino acid sequence set forth in SEQ ID NO: 517 (or a variant thereof); and a LCVR that comprises the amino acid sequence set forth in SEQ ID NO: 522 (or a variant thereof); and/or
(xxxii) a HCVR that comprises the amino acid sequence set forth in SEQ ID NO: 527 (or a variant thereof); and a LCVR that comprises the amino acid sequence set forth in SEQ ID NO: 532 (or a variant thereof).

238. The composition of any one of claims 231-237, wherein the anti-TfR antigen binding protein comprises:
(i) a HCVR that comprises the amino acid sequence set forth in SEQ ID NO: 437 (or a variant thereof); and a LCVR that comprises the amino acid sequence set forth in SEQ ID NO: 442 (or a variant thereof); or
(ii) a HCVR that comprises the amino acid sequence set forth in SEQ ID NO: 457 (or a variant thereof); and a LCVR that comprises the amino acid sequence set forth in SEQ ID NO: 462 (or a variant thereof).

239. The composition of any one of claims 218-238, wherein the TfR-binding delivery domain comprises an anti-TfR antibody, antibody fragment, or single-chain variable fragment (scFv).

240. The composition of claim 239, wherein the TfR-binding delivery domain is the single-chain variable fragment (scFv), optionally wherein the multidomain therapeutic protein comprises domains arranged in the following orientation: N′-Heavy chain variable region-Light chain variable region-lysosomal alpha-glucosidase-C′ or N′-Light chain variable region-Heavy chain variable region-lysosomal alpha-glucosidase-C′,
optionally wherein the scFv and lysosomal alpha-glucosidase are connected by a peptide linker, and optionally wherein the peptide linker which is -(GGGGS)ₘ- (SEQ ID NO: 600); wherein m is 1, 2, 3, 4, 5, 6, 7, 8, 9, or 10,
optionally wherein the scFv variable regions are connected by a peptide linker, and optionally wherein the peptide linker which is -(GGGGS)ₘ- (SEQ ID NO: 600); wherein m is 1, 2, 3, 4, 5, 6, 7, 8, 9, or 10.

241. The composition of claim 240, wherein the multidomain therapeutic protein comprises a heavy chain variable region (V_{H}) and a light chain variable region (V_{L}), and a lysosomal alpha-glucosidase, wherein the V_{H}, V_{L} and lysosomal alpha-glucosidase are arranged as follows:
(i) V_{L}-V_{L}-lysosomal alpha-glucosidase;
(ii) V_{H}-V_{L}-lysosomal alpha-glucosidase;
(iii) V_{L}-[(GGGGS)₃]-V_{H}-[(GGGGS)₂]-lysosomal alpha-glucosidase; or
(iv) V_{H}-[(GGGGS)₃]-V_{L}-[(GGGGS)₂]-lysosomal alpha-glucosidase.

242. The composition of claim 240 or 241, wherein the scFv comprises the sequence set forth in any one of SEQ ID NOS: 540, 549, 551, and 554,
optionally wherein the scFv comprises the sequence set forth in SEQ ID NO: 554.

243. The composition of any one of claims 240-242, wherein the scFv consists of the sequence set forth in any one of SEQ ID NOS: 540, 549, 551, and 554,
optionally wherein the scFv consists of the sequence set forth in SEQ ID NOS: 554.

244. The composition of any one of claims 218-243, wherein the scFv coding sequence is at least 90%, at least 91%, at least 92%, at least 93%, at least 94%, at least 95%, at least 96%, at least 97%, at least 98%, or at least 99% identical to any one of SEQ ID NOS: 587-599,
optionally wherein the scFv coding sequence is at least 90%, at least 91%, at least 92%, at least 93%, at least 94%, at least 95%, at least 96%, at least 97%, at least 98%, or at least 99% identical to any one of SEQ ID NOS: 593-595,
optionally wherein the scFv coding sequence is at least 90%, at least 91%, at least 92%, at least 93%, at least 94%, at least 95%, at least 96%, at least 97%, at least 98%, or at least 99% identical to SEQ ID NO: 593.

245. The composition of any one of claims 218-244, wherein the scFv coding sequence is at least 90%, at least 91%, at least 92%, at least 93%, at least 94%, at least 95%, at least 96%, at least 97%, at least 98%, or at least 99% identical to any one of SEQ ID NOS: 587-599 and encodes an scFv comprising any one of SEQ ID NOS: 540, 549, 551, and 554,
optionally wherein the scFv coding sequence is at least 90%, at least 91%, at least 92%, at least 93%, at least 94%, at least 95%, at least 96%, at least 97%, at least 98%, or at least 99% identical to any one of SEQ ID NOS: 593-595 and encodes an scFv comprising SEQ ID NO: 554,
optionally wherein the scFv coding sequence is at least 90%, at least 91%, at least 92%, at least 93%, at least 94%, at least 95%, at least 96%, at least 97%, at least 98%, or at least 99% identical to SEQ ID NO: 593 and encodes an scFv comprising SEQ ID NO: 554.

246. The composition of any one of claims 218-245, wherein the scFv coding sequence is at least 90%, at least 91%, at least 92%, at least 93%, at least 94%, at least 95%, at least 96%, at least 97%, at least 98%, or at least 99% identical to any one of SEQ ID NOS: 587-599, is codon-optimized and CpG-depleted, and encodes an scFv comprising any one of SEQ ID NOS: 540, 549, 551, and 554,
optionally wherein the scFv coding sequence is at least 90%, at least 91%, at least 92%, at least 93%, at least 94%, at least 95%, at least 96%, at least 97%, at least 98%, or at least 99% identical to any one of SEQ ID NOS: 593-595, is codon-optimized and CpG-depleted, and encodes an scFv comprising SEQ ID NO: 554,
optionally wherein the scFv coding sequence is at least 90%, at least 91%, at least 92%, at least 93%, at least 94%, at least 95%, at least 96%, at least 97%, at least 98%, or at least 99% identical to SEQ ID NO: 593, the scFv coding sequence is codon-optimized and CpG-depleted, and encodes an scFv comprising SEQ ID NO: 554.

247. The composition of any one of claims 218-246, wherein the scFv coding sequence comprises the sequence set forth in any one of SEQ ID NOS: 587-599,
optionally wherein the scFv coding sequence comprises the sequence set forth in any one of SEQ ID NOS: 593-595,
optionally wherein the scFv coding sequence comprises the sequence set forth in SEQ ID NO: 593.

248. The composition of any one of claims 218-247, wherein the scFv coding sequence consists of the sequence set forth in any one of SEQ ID NOS: 587-599,
optionally wherein the scFv coding sequence consists of the sequence set forth in any one of SEQ ID NOS: 593-595,
optionally wherein the scFv coding sequence consists of the sequence set forth in SEQ ID NO: 593.

249. The composition of any one of claims 218-248, wherein the coding sequence for the multidomain therapeutic protein is codon-optimized or CpG-depleted.

250. The composition of any one of claims 218-249, wherein the coding sequence for the multidomain therapeutic protein is codon-optimized and CpG-depleted.

251. The composition of any one of claims 218-250, wherein the multidomain therapeutic protein comprises the sequence set forth in any one of SEQ ID NOS: 570-573,
optionally wherein the multidomain therapeutic protein comprises the sequence set forth in SEQ ID NO: 573.

252. The composition of any one of claims 218-251, wherein the multidomain therapeutic protein consists of the sequence set forth in any one of SEQ ID NOS: 570-573,
optionally wherein the multidomain therapeutic protein consists of the sequence set forth in SEQ ID NO: 573.

253. The composition of any one of claims 218-252, wherein the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 574-586,
optionally wherein the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 584-586,
optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 584,
optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 733.

254. The composition of any one of claims 218-253, wherein the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 574-586, and the multidomain therapeutic protein comprises the sequence set forth in any one of SEQ ID NOS: 570-573,
optionally wherein the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 584-586, and the multidomain therapeutic protein comprises the sequence set forth in SEQ ID NO: 573,
optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 584, and the multidomain therapeutic protein comprises the sequence set forth in SEQ ID NO: 573,
optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 733, and the multidomain therapeutic protein comprises the sequence set forth in SEQ ID NO: 573.

255. The composition of any one of claims 218-254, wherein the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 574-586 and is codon-optimized and CpG-depleted, and the multidomain therapeutic protein comprises the sequence set forth in any one of SEQ ID NOS: 570-573,
optionally wherein the coding sequence for the multidomain therapeutic protein is at least 90%, at least 91%, at least 92%, at least 93%, at least 94%, at least 95%, at least 96%, at least 97%, at least 98%, or at least 99% identical to any one of SEQ ID NOS: 584-586 and is codon-optimized and CpG-depleted, and the multidomain therapeutic protein comprises the sequence set forth in SEQ ID NO: 573,
optionally wherein the coding sequence for the multidomain therapeutic protein is at least 90%, at least 91%, at least 92%, at least 93%, at least 94%, at least 95%, at least 96%, at least 97%, at least 98%, or at least 99% identical to SEQ ID NO: 584, the coding sequence for the multidomain therapeutic protein is codon-optimized and CpG-depleted, and the multidomain therapeutic protein comprises the sequence set forth in SEQ ID NO: 573,
optionally wherein the nucleic acid construct comprises a sequence at least 90%, at least 91%, at least 92%, at least 93%, at least 94%, at least 95%, at least 96%, at least 97%, at least 98%, or at least 99% identical to SEQ ID NO: 733, the coding sequence for the multidomain therapeutic protein is codon-optimized and CpG-depleted, and the multidomain therapeutic protein comprises the sequence set forth in SEQ ID NO: 573.

256. The composition of any one of claims 218-255, wherein the coding sequence for the multidomain therapeutic protein comprises the sequence set forth in any one of SEQ ID NOS: 574-586,
optionally wherein the coding sequence for the multidomain therapeutic protein comprises the sequence set forth in any one of SEQ ID NOS: 584-586,
optionally wherein the coding sequence for the multidomain therapeutic protein comprises the sequence set forth in SEQ ID NO: 584,
optionally wherein the nucleic acid construct comprises the sequence set forth in SEQ ID NO: 733.

257. The composition of any one of claims 218-256, wherein the coding sequence for the multidomain therapeutic protein consists of the sequence set forth in any one of SEQ ID NOS: 574-586,
optionally wherein the coding sequence for the multidomain therapeutic protein consists of the sequence set forth in any one of SEQ ID NOS: 584-586,
optionally wherein the coding sequence for the multidomain therapeutic protein consists of the sequence set forth in SEQ ID NO: 584,
optionally wherein the nucleic acid construct comprises the sequence set forth in SEQ ID NO: 733.

258. The composition of any one of claims 218-257, wherein the nucleic acid construct comprises a splice acceptor upstream of the coding sequence for the multidomain therapeutic protein.

259. The composition of any one of claims 218-258, wherein the nucleic acid construct comprises a polyadenylation signal or sequence downstream of the coding sequence for the multidomain therapeutic protein.

260. The composition of any one of claims 218-259, wherein the nucleic acid construct comprises a splice acceptor upstream of the coding sequence for the multidomain therapeutic protein, and the nucleic acid construct comprises a polyadenylation signal or sequence downstream of the coding sequence for the multidomain therapeutic protein.

261. The composition of any one of claims 218-260, wherein the nucleic acid construct does not comprise a homology arm.

262. The composition of any one of claims 218-260, wherein the nucleic acid construct comprises homology arms.

263. The composition of any one of claims 218-262, wherein the nucleic acid construct does not comprise a promoter that drives the expression of the multidomain therapeutic protein.

264. The composition of any one of claims 218-262, wherein the coding sequence for the multidomain therapeutic protein is operably linked to a promoter, optionally wherein the promoter is a liver-specific promoter.

265. The composition of any one of claims 218-264, wherein the nucleic acid construct is single-stranded DNA or double-stranded DNA, optionally wherein the nucleic acid construct is single-stranded DNA.

266. The composition of any one of claims 218-265, wherein the nucleic acid construct comprises from 5′ to 3′: a splice acceptor, the coding sequence for the multidomain therapeutic protein, and a polyadenylation signal or sequence,
wherein the lysosomal alpha-glucosidase coding sequence comprises the sequence set forth in any one of SEQ ID NOS: 174-182 and 205-212, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733,
wherein the nucleic acid construct does not comprise a promoter that drives the expression of the multidomain therapeutic protein, and
wherein the nucleic acid construct does not comprise a homology arm.

267. The composition of any one of claims 218-266, wherein the nucleic acid construct is in a nucleic acid vector or a lipid nanoparticle.

268. The composition of claim 267, wherein the nucleic acid construct is in the nucleic acid vector.

269. The composition of claim 268, wherein the nucleic acid vector is a viral vector.

270. The composition of claim 267 or 268, wherein the nucleic acid vector is an adeno-associated viral (AAV) vector,
optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

271. The composition of claim 270, wherein the AAV vector is a single-stranded AAV (ssAAV) vector.

272. The composition of claim 270 or 271, wherein the AAV vector is derived from an AAV8 vector, an AAV3B vector, an AAV5 vector, an AAV6 vector, an AAV7 vector, an AAV9 vector, an AAVrh.74 vector, or an AAVhu.37 vector.

273. The composition of claim 272, wherein the AAV vector is a recombinant AAV8 (rAAV8) vector.

274. The composition of claim 273, wherein the AAV vector is a single-stranded rAAV8 vector.

275. The composition of any one of claims 218-274, wherein the nucleic acid construct comprises from 5′ to 3′: a splice acceptor, the coding sequence for the multidomain therapeutic protein, and a polyadenylation signal or sequence,
wherein the lysosomal alpha-glucosidase coding sequence comprises the sequence set forth in any one of SEQ ID NOS: 174-182 and 205-212, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733,
wherein the nucleic acid construct does not comprise a promoter that drives the expression of the multidomain therapeutic protein,
wherein the nucleic acid construct does not comprise a homology arm, and
wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

276. The composition of any one of claims 218-275, wherein the nucleic acid construct is CpG-depleted.

277. The composition of any one of claims 218-276, further comprising a nuclease agent that targets a nuclease target site in a target genomic locus.

278. The composition of claim 277, wherein the target genomic locus is an albumin gene, optionally wherein the albumin gene is a human albumin gene.

279. The composition of claim 278, wherein the nuclease target site is in intron 1 of the albumin gene.

280. The composition of any one of claims 277-279, wherein the nuclease agent comprises:
(a) a zinc finger nuclease (ZFN);
(b) a transcription activator-like effector nuclease (TALEN); or
(c) (i) a Cas protein or a nucleic acid encoding the Cas protein; and
(ii) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

281. The composition of any one of claims 277-279, wherein the nuclease agent comprises:
(a) a Cas protein or a nucleic acid encoding the Cas protein; and
(b) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

282. The composition of claim 281, wherein the guide RNA target sequence is in intron 1 of an albumin gene.

283. The composition of claim 282, wherein the albumin gene is a human albumin gene.

284. The composition of any one of claims 281-283, wherein:
(I) the DNA-targeting segment comprises at least 17, at least 18, at least 19, or at least 20 contiguous nucleotides of the sequence set forth in any one of SEQ ID NOS: 30-61, optionally wherein the DNA-targeting segment comprises at least 17, at least 18, at least 19, or at least 20 contiguous nucleotides of the sequence set forth in any one of SEQ ID NOS: 36, 30, 33, and 41; and/or
(II) the DNA-targeting segment is at least 90% or at least 95% identical to the sequence set forth in any one of SEQ ID NOS: 30-61, optionally wherein the DNA-targeting segment is at least 90% or at least 95% identical to the sequence set forth in any one of SEQ ID NOS: 36, 30, 33, and 41.

285. The composition of any one of claims 281-284, wherein the DNA-targeting segment comprises any one of SEQ ID NOS: 30-61, optionally wherein the DNA-targeting segment comprises any one of SEQ ID NOS: 36, 30, 33, and 41.

286. The composition of any one of claims 281-285, wherein the DNA-targeting segment consists of any one of SEQ ID NOS: 30-61, optionally wherein the DNA-targeting segment consists of any one of SEQ ID NOS: 36, 30, 33, and 41.

287. The composition of any one of claims 281-286, wherein the guide RNA comprises any one of SEQ ID NOS: 62-125, optionally wherein the guide RNA comprises any one of SEQ ID NOS: 68, 100, 62, 94, 65, 97, 73, and 105.

288. The composition of any one of claims 281-287, wherein:
(I) the DNA-targeting segment comprises at least 17, at least 18, at least 19, or at least 20 contiguous nucleotides of SEQ ID NO: 36; and/or
(II) the DNA-targeting segment is at least 90% or at least 95% identical to SEQ ID NO: 36.

289. The composition of any one of claims 281-288, wherein the DNA-targeting segment comprises SEQ ID NO: 36.

290. The composition of any one of claims 281-289, wherein the DNA-targeting segment consists of SEQ ID NO: 36.

291. The composition of any one of claims 281-290, wherein the guide RNA comprises SEQ ID NO: 68 or 100.

292. The composition of any one of claims 281-291, wherein the guide RNA in the form of RNA.

293. The composition of any one of claims 281-292, wherein the guide RNA comprises at least one modification.

294. The composition of claim 293, wherein the at least one modification comprises a 2′-O-methyl-modified nucleotide.

295. The composition of claim 293 or 294, wherein the at least one modification comprises a phosphorothioate bond between nucleotides.

296. The composition of any one of claims 293-295, wherein the at least one modification comprises a modification at one or more of the first five nucleotides at the 5′ end of the guide RNA.

297. The composition of any one of claims 293-296, wherein the at least one modification comprises a modification at one or more of the last five nucleotides at the 3′ end of the guide RNA.

298. The composition of any one of claims 293-297, wherein the at least one modification comprises phosphorothioate bonds between the first four nucleotides at the 5′ end of the guide RNA.

299. The composition of any one of claims 293-298, wherein the at least one modification comprises phosphorothioate bonds between the last four nucleotides at the 3′ end of the guide RNA.

300. The composition of any one of claims 293-299, wherein the at least one modification comprises 2′-O-methyl-modified nucleotides at the first three nucleotides at the 5′ end of the guide RNA.

301. The composition of any one of claims 293-300, wherein the at least one modification comprises 2′-O-methyl-modified nucleotides at the last three nucleotides at the 3′ end of the guide RNA.

302. The composition of any one of claims 293-301, wherein the at least one modification comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

303. The composition of any one of claims 281-302, wherein the guide RNA is a single guide RNA (sgRNA).

304. The composition of any one of claims 281-303, wherein the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

305. The composition of any one of claims 281-304, wherein the Cas protein is a Cas9 protein.

306. The composition of claim 305, wherein the Cas9 protein is derived from a Streptococcus pyogenes Cas9 protein, a Staphylococcus aureus Cas9 protein, a Campylobacter jejuni Cas9 protein, a Streptococcus thermophilus Cas9 protein, or a Neisseria meningitidis Cas9 protein.

307. The composition of claim 305, wherein the Cas protein is derived from a Streptococcus pyogenes Cas9 protein.

308. The composition of any one of claims 281-307, wherein the Cas protein comprises the sequence set forth in SEQ ID NO: 11.

309. The composition of any one of claims 281-308, wherein the nucleic acid encoding the Cas protein is codon-optimized for expression in a mammalian cell or a human cell.

310. The composition of any one of claims 281-309, wherein the composition comprises the nucleic acid encoding the Cas protein, wherein the nucleic acid comprises an mRNA encoding the Cas protein.

311. The composition of claim 310, wherein the mRNA encoding the Cas protein comprises at least one modification.

312. The composition of claim 310 or 311, wherein the mRNA encoding the Cas protein is modified to comprise a modified uridine at one or more or all uridine positions.

313. The composition of claim 312, wherein the modified uridine is pseudouridine or N1-methyl-pseudouridine, optionally N1-methyl-pseudouridine.

314. The composition of claim 312 or 313, wherein the mRNA encoding the Cas protein is fully substituted with pseudouridine or N1-methyl-pseudouridine, optionally N1-methyl-pseudouridine.

315. The composition of any one of claims 310-314, wherein the mRNA encoding the Cas protein comprises a 5′ cap.

316. The composition of any one of claims 310-315, wherein the mRNA encoding the Cas protein comprises a polyadenylation sequence.

317. The composition of any one of claims 310-316, wherein the mRNA encoding the Cas protein comprises the sequence set forth in SEQ ID NO: 226, 225, or 12.

318. The composition of any one of claims 281-317, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine or N1-methyl-pseudouridine, optionally N1-methyl-pseudouridine, comprises a 5′ cap, and comprises a polyadenylation sequence.

319. The composition of any one of claims 281-318, wherein the guide RNA in the form of RNA, and the guide RNA comprises SEQ ID NO: 68 or 100, and
wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 226, 225, or 12.

320. The composition of claim 319, wherein the nucleic acid construct comprises from 5′ to 3′: a splice acceptor, the coding sequence for the multidomain therapeutic protein, and a polyadenylation signal or sequence,
wherein the lysosomal alpha-glucosidase coding sequence comprises the sequence set forth in any one of SEQ ID NOS: 174-182 and 205-212, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733,
wherein the nucleic acid construct does not comprise a promoter that drives the expression of the multidomain therapeutic protein,
wherein the nucleic acid construct does not comprise a homology arm, and
wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

321. The composition of any one of claims 281-318, wherein the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and
wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine or N1-methyl-pseudouridine, optionally N1-methyl-pseudouridine, comprises a 5′ cap, and comprises a polyadenylation sequence.

322. The composition of claim 321, wherein the nucleic acid construct comprises from 5′ to 3′: a splice acceptor, the coding sequence for the multidomain therapeutic protein, and a polyadenylation signal or sequence,
wherein the lysosomal alpha-glucosidase coding sequence comprises the sequence set forth in any one of SEQ ID NOS: 174-182 and 205-212, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733,
wherein the nucleic acid construct does not comprise a promoter that drives the expression of the multidomain therapeutic protein,
wherein the nucleic acid construct does not comprise a homology arm, and
wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

323. The composition of any one of claims 281-322, wherein the Cas protein or the nucleic acid encoding the Cas protein and the guide RNA or the one or more DNAs encoding the guide RNA are associated with a lipid nanoparticle.

324. The composition of claim 323, wherein the lipid nanoparticle comprises a cationic lipid, a neutral lipid, a helper lipid, and a stealth lipid.

325. The composition of claim 324, wherein the cationic lipid is Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate).

326. The composition of claim 324 or 325, wherein the neutral lipid is distearoylphosphatidylcholine or 1,2-distearoyl-sn-glycero-3-phosphocholine (DSPC).

327. The composition of any one of claims 324-326, wherein the helper lipid is cholesterol.

328. The composition of any one of claims 324-327, wherein the stealth lipid is 1,2-dimyristoyl-rac-glycero-3-methoxypolyethylene glycol-2000 (PEG2k-DMG).

329. The composition of any one of claims 324-328, wherein the cationic lipid is Lipid A, the neutral lipid is DSPC, the helper lipid is cholesterol, and the stealth lipid is PEG2k-DMG.

330. The composition of any one of claims 323-329, wherein the lipid nanoparticle comprises four lipids at the following molar ratios: about 50 mol% Lipid A, about 9 mol% DSPC, about 38 mol% cholesterol, and about 3 mol% PEG2k-DMG.

331. The composition of any one of claims 281-330, wherein the albumin gene is a human albumin gene,
wherein the guide RNA in the form of RNA, and the guide RNA comprises SEQ ID NO: 68 or 100,
wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 226, 225, or 12, and
wherein the guide RNA and the mRNA encoding the Cas protein are associated with a lipid nanoparticle comprising Lipid A, DSPC, cholesterol, and PEG2k-DMG, optionally at the following molar ratios: about 50 mol% Lipid A, about 9 mol% DSPC, about 38 mol% cholesterol, and about 3 mol% PEG2k-DMG.

332. The composition of claim 331, wherein the nucleic acid construct comprises from 5′ to 3′: a splice acceptor, the coding sequence for the multidomain therapeutic protein, and a polyadenylation signal or sequence,
wherein the lysosomal alpha-glucosidase coding sequence comprises the sequence set forth in any one of SEQ ID NOS: 174-182 and 205-212, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733,
wherein the nucleic acid construct does not comprise a promoter that drives the expression of the multidomain therapeutic protein,
wherein the nucleic acid construct does not comprise a homology arm, and
wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

333. The composition of any one of claims 281-330, wherein the albumin gene is a human albumin gene,
wherein the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA,
wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine or N1-methyl-pseudouridine, optionally N1-methyl-pseudouridine, comprises a 5′ cap, and comprises a polyadenylation sequence, and
wherein the guide RNA and the mRNA encoding the Cas protein are associated with a lipid nanoparticle comprising Lipid A, DSPC, cholesterol, and PEG2k-DMG, optionally at the following molar ratios: about 50 mol% Lipid A, about 9 mol% DSPC, about 38 mol% cholesterol, and about 3 mol% PEG2k-DMG.

334. The composition of claim 333, wherein the nucleic acid construct comprises from 5′ to 3′: a splice acceptor, the coding sequence for the multidomain therapeutic protein, and a polyadenylation signal or sequence,
wherein the lysosomal alpha-glucosidase coding sequence comprises the sequence set forth in any one of SEQ ID NOS: 174-182 and 205-212, optionally wherein the lysosomal alpha-glucosidase coding sequence comprises the sequence set forth in SEQ ID NO: 176, optionally wherein the coding sequence for the multidomain therapeutic protein comprises the sequence set forth in any one of SEQ ID NOS: 574-586, and optionally wherein the coding sequence for the multidomain therapeutic protein comprises the sequence set forth in any one of SEQ ID NOS: 584-586, optionally wherein the coding sequence for the multidomain therapeutic protein comprises the sequence set forth in SEQ ID NO: 584, optionally wherein the nucleic acid construct comprises the sequence set forth in SEQ ID NO: 733,
wherein the nucleic acid construct does not comprise a promoter that drives the expression of the multidomain therapeutic protein,
wherein the nucleic acid construct does not comprise a homology arm, and
wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

335. The composition of any one of claims 218-334, for use in a method of inserting the nucleic acid encoding the multidomain therapeutic protein into a target genomic locus in a cell or a population of cells.

336. The composition of any one of claims 218-334, for use in a method of expressing the multidomain therapeutic protein from a target genomic locus in a cell or a population of cells or for use in a method of expressing the multidomain therapeutic protein in a cell or a population of cells.

337. The composition of any one of claims 218-334, for use in a method of inserting the nucleic acid encoding the multidomain therapeutic protein into a target genomic locus in a cell or a population of cells in a subject.

338. The composition of any one of claims 218-334, for use in a method of expressing the multidomain therapeutic protein from a target genomic locus in a cell or a population of cells in a subject or for use in a method of expressing the multidomain therapeutic protein in a cell or a population of cells in a subject.

339. The composition of any one of claims 218-334, for use in a method of treating a lysosomal alpha-glucosidase deficiency in a subject in need thereof.

340. The composition of any one of claims 218-334, for use in a method of reducing glycogen accumulation in a tissue in a subject in need thereof.

341. The composition of any one of claims 218-334, for use in a method of treating Pompe disease in a subject in need thereof.

342. The composition of any one of claims 218-334, for use in a method of preventing or reducing the onset of a sign or symptom of Pompe disease in a neonatal subject in need thereof.

343. A cell comprising the composition of any one of claims 218-334.

344. The cell of claim 343, wherein the nucleic acid construct is integrated into a target genomic locus, and wherein the multidomain therapeutic protein is expressed from the target genomic locus, or wherein the nucleic acid construct is integrated into intron 1 of an endogenous albumin locus, and wherein the multidomain therapeutic protein is expressed from the endogenous albumin locus.

345. The cell of claim 343 or 344, wherein the cell is a liver cell.

346. The cell of any one of claims 343-345, wherein the liver cell is a hepatocyte.

347. The cell of any one of claims 343-346, wherein the cell is a human cell.

348. The cell of any one of claims 343-347, wherein the cell is a neonatal cell.

349. The cell of claim 348, wherein the neonatal cell is from a human neonatal subject within 24 weeks after birth.

350. The cell of claim 348, wherein the neonatal cell is from a human neonatal subject within 12 weeks after birth.

351. The cell of claim 348, wherein the neonatal cell is from a human neonatal subject within 8 weeks after birth.

352. The cell of claim 348, wherein the neonatal cell is from a human neonatal subject within 4 weeks after birth.

353. The cell of any one of claims 343-347, wherein the cell is not a neonatal cell.

354. The cell of any one of claims 343-353, wherein the cell is in vivo.

355. The cell of any one of claims 343-353, wherein the cell is in vitro or ex vivo.

356. A method of inserting a nucleic acid encoding a multidomain therapeutic protein comprising a TfR-binding delivery domain fused to a lysosomal alpha-glucosidase into a target genomic locus in a cell or a population of cells, comprising administering to the cell or the population of cells the composition of any one of claims 277-334,
wherein the nuclease agent cleaves the nuclease target site in the target genomic locus, and the nucleic acid construct is inserted into the target genomic locus.

357. A method of expressing a multidomain therapeutic protein comprising a TfR-binding delivery domain fused to a lysosomal alpha-glucosidase in a cell or a population of cells, comprising administering to the cell or the population of cells the composition of any one of claims 218-276,
wherein the coding sequence for the multidomain therapeutic protein is operably linked to a promoter in the nucleic acid construct and is expressed in the cell or population of cells.

358. A method of expressing a multidomain therapeutic protein comprising a TfR-binding delivery domain fused to a lysosomal alpha-glucosidase from a target genomic locus in a cell or a population of cells, comprising administering to the cell or the population of cells the composition of any one of claims 277-334,
wherein the nuclease agent cleaves the nuclease target site in the target genomic locus, the nucleic acid construct is inserted into the target genomic locus to create a modified target genomic locus, and the multidomain therapeutic protein comprising the TfR-binding delivery domain fused to the lysosomal alpha-glucosidase is expressed from the modified target genomic locus.

359. The method of any one of claims 356-358, wherein the cell is a liver cell or the population of cells is a population of liver cells, optionally wherein the cell is a hepatocyte or the population of cells is a population of hepatocytes.

360. The method of any one of claims 356-359, wherein the cell is a human cell or the population of cells is a population of human cells.

361. The method of any one of claims 356-360, wherein the cell is a neonatal cell or the population of cells is a population of neonatal cells.

362. The method of claim 361, wherein the neonatal cell or the population of neonatal cells is from a human neonatal subject within 24 weeks after birth.

363. The method of claim 361, wherein the neonatal cell or the population of neonatal cells is from a human neonatal subject within 12 weeks after birth.

364. The method of claim 361, wherein the neonatal cell or the population of neonatal cells is from a human neonatal subject within 8 weeks after birth.

365. The method of claim 361, wherein the neonatal cell or the population of neonatal cells is from a human neonatal subject within 4 weeks after birth.

366. The method of any one of claims 356-360, wherein the cell is not a neonatal cell or the population of cells is not a population of neonatal cells.

367. The method of any one of claims 356-366, wherein the cell is in vitro or ex vivo or the population of cells is in vitro or ex vivo.

368. The method of any one of claims 356-366, wherein the cell is in vivo in a subject or the population of cells is in vivo in a subject.

369. A method of inserting a nucleic acid encoding a multidomain therapeutic protein comprising a TfR-binding delivery domain fused to a lysosomal alpha-glucosidase into a target genomic locus in a cell in a subject, comprising administering to the subject the composition of any one of claims 277-334,
wherein the nuclease agent cleaves the nuclease target site in the target genomic locus, and the nucleic acid construct is inserted into the target genomic locus.

370. A method of expressing a multidomain therapeutic protein comprising a TfR-binding delivery domain fused to a lysosomal alpha-glucosidase protein in a cell in a subject, comprising administering to the subject the composition of any one of claims 218-276,
wherein the coding sequence for the multidomain therapeutic protein is operably linked to a promoter in the nucleic acid construct and is expressed in the cell.

371. A method of expressing a multidomain therapeutic protein comprising a TfR-binding delivery domain fused to a lysosomal alpha-glucosidase protein from a target genomic locus in a cell in a subject, comprising administering to the subject the composition of any one of claims 277-334,
wherein the nuclease agent cleaves the nuclease target site in the target genomic locus, the nucleic acid construct is inserted into the target genomic locus to create a modified target genomic locus, and the multidomain therapeutic protein comprising the TfR-binding delivery domain fused to the lysosomal alpha-glucosidase is expressed from the modified target genomic locus.

372. The method of claim 370, wherein the expressed multidomain therapeutic protein is delivered to and internalized by skeletal muscle, heart, and central nervous system tissue in the subject.

373. The method of any one of claims 369-372, wherein the cell is a liver cell, optionally wherein the cell is a hepatocyte.

374. The method of any one of claims 369-373, wherein the cell is a human cell.

375. The method of any one of claims 369-374, wherein the cell is a neonatal cell.

376. The method of claim 375, wherein the neonatal subject is a human subject within 24 weeks after birth.

377. The method of claim 375, wherein the neonatal subject is a human subject within 12 weeks after birth.

378. The method of claim 375, wherein the neonatal subject is a human subject within 8 weeks after birth.

379. The method of claim 375, wherein the neonatal subject is a human subject within 4 weeks after birth.

380. The method of any one of claims 369-374, wherein the cell is not a neonatal cell.

381. A method of treating a lysosomal alpha-glucosidase deficiency in a subject in need thereof, comprising administering to the subject the composition of any one of claims 218-276,
wherein the coding sequence for the multidomain therapeutic protein is operably linked to a promoter in the nucleic acid construct and is expressed in the subject.

382. A method of treating a lysosomal alpha-glucosidase deficiency in a subject in need thereof, comprising administering to the subject the composition of any one of claims 277-334,
wherein the nuclease agent cleaves the nuclease target site in the target genomic locus, the nucleic acid construct is inserted into the target genomic locus to create a modified target genomic locus, and the multidomain therapeutic protein comprising the TfR-binding delivery domain fused to the lysosomal alpha-glucosidase is expressed from the modified target genomic locus.

383. A method of reducing glycogen accumulation in a tissue in a subject in need thereof, comprising administering to the subject the composition of any one of claims 218-276,
wherein the coding sequence for the multidomain therapeutic protein is operably linked to a promoter in the nucleic acid construct and is expressed in the subject and reduces glycogen accumulation in the tissue.

384. A method of reducing glycogen accumulation in a tissue in a subject in need thereof, comprising administering to the subject the composition of any one of claims 277-334,
wherein the nuclease agent cleaves the nuclease target site in the target genomic locus, the nucleic acid construct is inserted into the target genomic locus to create a modified target genomic locus, and the multidomain therapeutic protein comprising the TfR-binding delivery domain fused to the lysosomal alpha-glucosidase is expressed from the modified target genomic locus and reduces glycogen accumulation in the tissue.

385. The method of any one of claims 368-384, wherein the subject has Pompe disease.

386. A method of treating Pompe disease in a subject in need thereof, comprising administering to the subject the composition of any one of claims 218-276,
wherein the coding sequence for the multidomain therapeutic protein is operably linked to a promoter in the nucleic acid construct and is expressed in the subject, thereby treating the Pompe disease.

387. A method of treating Pompe disease in a subject in need thereof, comprising administering to the subject the composition of any one of claims 277-334,
wherein the nuclease agent cleaves the nuclease target site in the target genomic locus, the nucleic acid construct is inserted into the target genomic locus to create a modified target genomic locus, and the multidomain therapeutic protein comprising the TfR-binding delivery domain fused to the lysosomal alpha-glucosidase is expressed from the modified target genomic locus, thereby treating the Pompe disease.

388. The method of any one of claims 385-387, wherein the Pompe disease is infantile-onset Pompe disease.

389. The method of any one of claims 385-387, wherein the Pompe disease is late-onset Pompe disease.

390. The method of any one of claims 368-389, wherein the subject is a human subject.

391. The method of any one of claims 368-390, wherein the subject is a neonatal subject.

392. The method of claim 391, wherein the neonatal subject is a human subject within 24 weeks after birth, optionally wherein the neonatal subject is a human subject within 12 weeks after birth, within 8 weeks after birth, or within 4 weeks after birth.

393. The method of any one of claims 368-390, wherein the subject is not a neonatal subject.

394. The method of any one of claims 368-393, wherein the method results in a therapeutically effective level of circulating multidomain therapeutic protein or lysosomal alpha-glucosidase in the subject.

395. The method of any one of claims 368-394, wherein the method reduces glycogen accumulation in skeletal muscle, heart, or central nervous system tissue in the subject.

396. The method of claim 395, wherein the method reduces glycogen accumulation in skeletal muscle, heart, and central nervous system tissue in the subject.

397. The method of claim 396, wherein the method results in reduced glycogen levels in skeletal muscle, heart, and central nervous system tissue in the subject comparable to wild type levels at the same age.

398. The method of any one of claims 368-397, wherein the method improves muscle strength in the subject or prevents loss of muscle strength in the subject compared to a control subject.

399. The method of claim 398, wherein the method results in the subject having muscle strength comparable to wild type levels at the same age.

400. A method of preventing or reducing the onset of a sign or symptom of Pompe disease in a neonatal subject in need thereof, comprising administering to the neonatal subject the composition of any one of claims 218-276,
wherein the coding sequence for the multidomain therapeutic protein is operably linked to a promoter in the nucleic acid construct and is expressed in the subject, thereby preventing or reducing the onset of a sign or symptom of the Pompe disease in the subject.

401. A method of preventing or reducing the onset of a sign or symptom of Pompe disease in a neonatal subject in need thereof, comprising administering to the neonatal subject the composition of any one of claims 277-334,
wherein the nuclease agent cleaves the nuclease target site, the nucleic acid construct is inserted into the target genomic locus to create a modified target genomic locus, and the multidomain therapeutic protein comprising the TfR-binding delivery domain fused to the lysosomal alpha-glucosidase is expressed from the modified target genomic locus, thereby preventing or reducing the onset of a sign or symptom of the Pompe disease in the subject.

402. The method of claim 400 or 401, wherein the Pompe disease is infantile-onset Pompe disease.

403. The method of claim 400 or 401, wherein the Pompe disease is late-onset Pompe disease.

404. The method of any one of claims 400-403, wherein the method results in a therapeutically effective level of circulating multidomain therapeutic protein or lysosomal alpha-glucosidase in the subject.

405. The method of any one of claims 400-404, wherein the method prevents or reduces glycogen accumulation in skeletal muscle, heart, or central nervous system tissue in the subject.

406. The method of any one of claims 400-405, wherein the method prevents or reduces glycogen accumulation in skeletal muscle, heart, and central nervous system tissue in the subject.

407. The method of any one of claims 400-406, wherein the subject is a human subject.

408. The method of any one of claims 400-407, wherein the subject is a neonatal subject.

409. The method of claim 408, wherein the neonatal subject is a human subject within 24 weeks after birth, optionally wherein the neonatal subject is a human subject within 12 weeks after birth.

410. The method of claim 408, wherein the neonatal subject is a human subject within 8 weeks after birth.

411. The method of claim 408, wherein the neonatal subject is a human subject within 4 weeks after birth.

412. The method of any one of claims 368-407, wherein the subject is not a neonatal subject.

413. The method of any one of claims 368-412, wherein the method results in increased expression of the multidomain therapeutic protein in the subject compared to a method comprising administering an episomal expression vector encoding the multidomain therapeutic protein to a control subject.

414. The method of any one of claims 368-413, wherein the method results in increased serum levels of the multidomain therapeutic protein in the subject compared to a method comprising administering an episomal expression vector encoding the multidomain therapeutic protein to a control subject.

415. The method of any one of claims 368-414, wherein the method results in serum levels of the multidomain therapeutic protein in the subject of at least about 1 µg/mL, at least about 2 µg/mL, at least about 3 µg/mL, at least about 4 µg/mL, at least about 5 µg/mL, at least about 6 µg/mL, at least about 7 µg/mL, at least about 8 µg/mL, at least about 9 µg/mL, or at least about 10 µg/mL.

416. The method of any one of claims 368-415, wherein the method results in serum levels of the multidomain therapeutic protein in the subject of at least about 2 µg/mL or at least about 5 µg/mL.

417. The method of any one of claims 368-416, wherein the method results in serum levels of the multidomain therapeutic protein in the subject of between about 2 µg/mL and about 30 µg/mL or between about 2 µg/mL and about 20 µg/mL.

418. The method of any one of claims 368-417, wherein the method results in serum levels of the multidomain therapeutic protein in the subject of between about 5 µg/mL and about 30 µg/mL or between about 5 µg/mL and about 20 µg/mL.

419. The method of any one of claims 368-418, wherein the method achieves lysosomal alpha-glucosidase activity levels of at least about 40% of normal, at least about 45% of normal, at least about 50%, at least about 60%, at least about 70%, at least about 80%, at least about 90%, or 100% of normal.

420. The method of any one of claims 368-419, wherein:
(I) the subject has infantile-onset Pompe disease, and the method achieves lysosomal alpha-glucosidase expression or activity levels of at least about 1% or more than about 1% of normal; or
(II) the subject has late-onset Pompe disease, and the method achieves lysosomal alpha-glucosidase expression or activity levels of at least about 40% of normal or more than about 40% of normal.

421. The method of any one of claims 368-420, wherein the expression or activity of the multidomain therapeutic protein is at least 50% of the expression or activity of the multidomain therapeutic protein at a peak level of expression measured for the subject at 24 weeks after the administering.

422. The method of any one of claims 368-421, wherein the expression or activity of the multidomain therapeutic protein is at least 50% of the expression or activity of the multidomain therapeutic protein at a peak level of expression measured for the subject at one year after the administering.

423. The method of any one of claims 368-422, wherein the expression or activity of the multidomain therapeutic protein is at least 60% of the expression or activity of the multidomain therapeutic protein at a peak level of expression measured for the subject at 24 weeks after the administering.

424. The method of any one of claims 368-423, wherein the expression or activity of the multidomain therapeutic protein is at least 50% of the expression or activity of the multidomain therapeutic protein at a peak level of expression measured for the subject at two years after the administering.

425. The method of any one of claims 368-424, wherein the expression or activity of the multidomain therapeutic protein is at least 60% of the expression or activity of the multidomain therapeutic protein at a peak level of expression measured for the subject at 2 years after the administering.

426. The method of any one of claims 368-425, wherein the expression or activity of the multidomain therapeutic protein is at least 60% of the expression or activity of the multidomain therapeutic protein at a peak level of expression measured for the subject at 24 weeks after the administering.

427. The method of any one of claim 368-426, wherein the method further comprises assessing preexisting AAV immunity in the subject prior to administering the nucleic acid construct to the subject.

428. The method of claim 427, wherein the preexisting AAV immunity is preexisting AAV8 immunity.

429. The method of claim 427 or 428, wherein assessing preexisting AAV immunity comprises assessing immunogenicity using a total antibody immune assay or a neutralizing antibody assay.

430. The method of any one of claims 356-429, wherein the nucleic acid construct is administered simultaneously with the nuclease agent or the one or more nucleic acids encoding the nuclease agent.

431. The method of any one of claims 356-429, wherein the nucleic acid construct is not administered simultaneously with the nuclease agent or the one or more nucleic acids encoding the nuclease agent.

432. The method of claim 431, wherein the nucleic acid construct is administered prior to the nuclease agent or the one or more nucleic acids encoding the nuclease agent.

433. The method of claim 431, wherein the nucleic acid construct is administered after the nuclease agent or the one or more nucleic acids encoding the nuclease agent.

434. A composition comprising a nucleic acid construct comprising a coding sequence for lysosomal alpha-glucosidase, wherein the lysosomal alpha-glucosidase coding sequence is CpG-depleted relative to a wild type lysosomal alpha-glucosidase coding sequence.

435. The composition of claim 434, wherein the lysosomal alpha-glucosidase lacks the lysosomal alpha-glucosidase signal peptide and propeptide.

436. The composition of claim 434 or 435, wherein the lysosomal alpha-glucosidase comprises the sequence set forth in SEQ ID NO: 173.

437. The composition of any one of claims 434-436, wherein the lysosomal alpha-glucosidase consists of the sequence set forth in SEQ ID NO: 173.

438. The composition of any one of claims 434-437, wherein the lysosomal alpha-glucosidase coding sequence is codon-optimized and CpG-depleted.

439. The composition of any one of claims 434-438, wherein the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and 205-212,
optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176.

440. The composition of any one of claims 434-439, wherein the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and 205-212 and encodes a lysosomal alpha-glucosidase protein comprising SEQ ID NO: 173,
optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176 and encodes a lysosomal alpha-glucosidase protein comprising SEQ ID NO: 173.

441. The composition of any one of claims 434-440, wherein the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NOS: 174-182 and 205-212, is codon-optimized and CpG-depleted, and encodes a lysosomal alpha-glucosidase protein comprising SEQ ID NO: 173,
optionally wherein the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 176, is codon-optimized and CpG-depleted, and encodes a lysosomal alpha-glucosidase protein comprising SEQ ID NO: 173.

442. The composition of any one of claims 434-441, wherein the lysosomal alpha-glucosidase coding sequence comprises the sequence set forth in any one of SEQ ID NOS: 174-182 and 205-212,
optionally wherein the lysosomal alpha-glucosidase coding sequence comprises the sequence set forth in SEQ ID NO: 176.

443. The composition of any one of claims 434-442, wherein the lysosomal alpha-glucosidase coding sequence consists of the sequence set forth in any one of SEQ ID NOS: 174-182 and 205-212,
optionally wherein the lysosomal alpha-glucosidase coding sequence consists of the sequence set forth in SEQ ID NO: 176.

444. The composition of any one of claims 434-443, wherein the nucleic acid construct comprises a splice acceptor upstream of the lysosomal alpha-glucosidase coding sequence.

445. The composition of any one of claims 434-444, wherein the nucleic acid construct comprises a polyadenylation signal or sequence downstream of the lysosomal alpha-glucosidase coding sequence.

446. The composition of any one of claims 434-445, wherein the nucleic acid construct comprises a splice acceptor upstream of the lysosomal alpha-glucosidase coding sequence, and the nucleic acid construct comprises a polyadenylation signal or sequence downstream of the lysosomal alpha-glucosidase coding sequence.

447. The composition of any one of claims 434-446, wherein the nucleic acid construct does not comprise a homology arm.

448. The composition of any one of claims 434-446, wherein the nucleic acid construct comprises homology arms.

449. The composition of any one of claims 434-448, wherein the nucleic acid construct does not comprise a promoter that drives the expression of the lysosomal alpha-glucosidase.

450. The composition of any one of claims 434-448, wherein the lysosomal alpha-glucosidase coding sequence is operably linked to a promoter, optionally wherein the promoter is a liver-specific promoter.

451. The composition of any one of claims 434-450, wherein the nucleic acid construct is single-stranded DNA or double-stranded DNA, optionally wherein the nucleic acid construct is single-stranded DNA.

452. The composition of any one of claims 434-451, wherein the nucleic acid construct comprises from 5′ to 3′: a splice acceptor, the lysosomal alpha-glucosidase coding sequence, and a polyadenylation signal or sequence,
wherein the lysosomal alpha-glucosidase coding sequence comprises any one of SEQ ID NOS: 174-182 and 205-212, optionally wherein the lysosomal alpha-glucosidase coding sequence comprises SEQ ID NO: 176,
wherein the nucleic acid construct does not comprise a promoter that drives the expression of the lysosomal alpha-glucosidase, and
wherein the nucleic acid construct does not comprise a homology arm.

453. The composition of any one of claims 434-452, wherein the nucleic acid construct is in a nucleic acid vector or a lipid nanoparticle.

454. The composition of claim 453, wherein the nucleic acid construct is in the nucleic acid vector.

455. The composition of claim 454, wherein the nucleic acid vector is a viral vector.

456. The composition of claim 453 or 454, wherein the nucleic acid vector is an adeno-associated viral (AAV) vector,
optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

457. The composition of claim 456, wherein the AAV vector is a single-stranded AAV (ssAAV) vector.

458. The composition of claim 456 or 457, wherein the AAV vector is derived from an AAV8 vector, an AAV3B vector, an AAV5 vector, an AAV6 vector, an AAV7 vector, an AAV9 vector, an AAVrh.74 vector, or an AAVhu.37 vector.

459. The composition of claim 458, wherein the AAV vector is a recombinant AAV8 (rAAV8) vector.

460. The composition of claim 459, wherein the AAV vector is a single-stranded rAAV8 vector.

461. The composition of any one of claims 434-460, wherein the nucleic acid construct comprises from 5′ to 3′: a splice acceptor, the lysosomal alpha-glucosidase coding sequence, and a polyadenylation signal or sequence,
wherein the lysosomal alpha-glucosidase coding sequence comprises any one of SEQ ID NOS: 174-182 and 205-212, optionally wherein the lysosomal alpha-glucosidase coding sequence comprises SEQ ID NO: 176,
wherein the nucleic acid construct does not comprise a promoter that drives the expression of the lysosomal alpha-glucosidase,
wherein the nucleic acid construct does not comprise a homology arm, and
wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

462. The composition of any one of claims 434-461, wherein the nucleic acid construct is CpG-depleted.

463. The composition of any one of claims 434-462, further comprising a nuclease agent that targets a nuclease target site in a target genomic locus.

464. The composition of claim 463, wherein the target genomic locus is an albumin gene, optionally wherein the albumin gene is a human albumin gene.

465. The composition of claim 464, wherein the nuclease target site is in intron 1 of the albumin gene.

466. The composition of any one of claims 463-465, wherein the nuclease agent comprises:
(a) a zinc finger nuclease (ZFN);
(b) a transcription activator-like effector nuclease (TALEN); or
(c) (i) a Cas protein or a nucleic acid encoding the Cas protein; and
(ii) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

467. The composition of any one of claims 463-465, wherein the nuclease agent comprises:
(a) a Cas protein or a nucleic acid encoding the Cas protein; and
(b) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

468. The composition of claim 467, wherein the guide RNA target sequence is in intron 1 of an albumin gene.

469. The composition of claim 468, wherein the albumin gene is a human albumin gene.

470. The composition of any one of claims 467-469, wherein:
(I) the DNA-targeting segment comprises at least 17, at least 18, at least 19, or at least 20 contiguous nucleotides of the sequence set forth in any one of SEQ ID NOS: 30-61, optionally wherein the DNA-targeting segment comprises at least 17, at least 18, at least 19, or at least 20 contiguous nucleotides of the sequence set forth in any one of SEQ ID NOS: 36, 30, 33, and 41; and/or
(II) the DNA-targeting segment is at least 90% or at least 95% identical to the sequence set forth in any one of SEQ ID NOS: 30-61, optionally wherein the DNA-targeting segment is at least 90% or at least 95% identical to the sequence set forth in any one of SEQ ID NOS: 36, 30, 33, and 41.

471. The composition of any one of claims 467-470, wherein the DNA-targeting segment comprises any one of SEQ ID NOS: 30-61, optionally wherein the DNA-targeting segment comprises any one of SEQ ID NOS: 36, 30, 33, and 41.

472. The composition of any one of claims 467-471, wherein the DNA-targeting segment consists of any one of SEQ ID NOS: 30-61, optionally wherein the DNA-targeting segment consists of any one of SEQ ID NOS: 36, 30, 33, and 41.

473. The composition of any one of claims 467-472, wherein the guide RNA comprises any one of SEQ ID NOS: 62-125, optionally wherein the guide RNA comprises any one of SEQ ID NOS: 68, 100, 62, 94, 65, 97, 73, and 105.

474. The composition of any one of claims 467-473, wherein:
(I) the DNA-targeting segment comprises at least 17, at least 18, at least 19, or at least 20 contiguous nucleotides of SEQ ID NO: 36; and/or
(II) the DNA-targeting segment is at least 90% or at least 95% identical to SEQ ID NO: 36.

475. The composition of any one of claims 467-474, wherein the DNA-targeting segment comprises SEQ ID NO: 36.

476. The composition of any one of claims 467-475, wherein the DNA-targeting segment consists of SEQ ID NO: 36.

477. The composition of any one of claims 467-476, wherein the guide RNA comprises SEQ ID NO: 68 or 100.

478. The composition of any one of claims 467-477, wherein the guide RNA in the form of RNA.

479. The composition of any one of claims 467-478, wherein the guide RNA comprises at least one modification.

480. The composition of claim 479, wherein the at least one modification comprises a 2′-O-methyl-modified nucleotide.

481. The composition of claim 479 or 480, wherein the at least one modification comprises a phosphorothioate bond between nucleotides.

482. The composition of any one of claims 479-481, wherein the at least one modification comprises a modification at one or more of the first five nucleotides at the 5′ end of the guide RNA.

483. The composition of any one of claims 479-482, wherein the at least one modification comprises a modification at one or more of the last five nucleotides at the 3′ end of the guide RNA.

484. The composition of any one of claims 479-483, wherein the at least one modification comprises phosphorothioate bonds between the first four nucleotides at the 5′ end of the guide RNA.

485. The composition of any one of claims 479-484, wherein the at least one modification comprises phosphorothioate bonds between the last four nucleotides at the 3′ end of the guide RNA.

486. The composition of any one of claims 479-485, wherein the at least one modification comprises 2′-O-methyl-modified nucleotides at the first three nucleotides at the 5′ end of the guide RNA.

487. The composition of any one of claims 479-486, wherein the at least one modification comprises 2′-O-methyl-modified nucleotides at the last three nucleotides at the 3′ end of the guide RNA.

488. The composition of any one of claims 479-487, wherein the at least one modification comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

489. The composition of any one of claims 467-488, wherein the guide RNA is a single guide RNA (sgRNA).

490. The composition of any one of claims 467-489, wherein the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

491. The composition of any one of claims 467-490, wherein the Cas protein is a Cas9 protein.

492. The composition of claim 491, wherein the Cas9 protein is derived from a Streptococcus pyogenes Cas9 protein, a Staphylococcus aureus Cas9 protein, a Campylobacter jejuni Cas9 protein, a Streptococcus thermophilus Cas9 protein, or a Neisseria meningitidis Cas9 protein.

493. The composition of claim 491, wherein the Cas protein is derived from a Streptococcus pyogenes Cas9 protein.

494. The composition of any one of claims 467-493, wherein the Cas protein comprises the sequence set forth in SEQ ID NO: 11.

495. The composition of any one of claims 467-494, wherein the nucleic acid encoding the Cas protein is codon-optimized for expression in a mammalian cell or a human cell.

496. The composition of any one of claims 467-495, wherein the composition comprises the nucleic acid encoding the Cas protein, wherein the nucleic acid comprises an mRNA encoding the Cas protein.

497. The composition of claim 496, wherein the mRNA encoding the Cas protein comprises at least one modification.

498. The composition of claim 496 or 497, wherein the mRNA encoding the Cas protein is modified to comprise a modified uridine at one or more or all uridine positions.

499. The composition of claim 498, wherein the modified uridine is pseudouridine or N1-methyl-pseudouridine, optionally N1-methyl-pseudouridine.

500. The composition of claim 498 or 499, wherein the mRNA encoding the Cas protein is fully substituted with pseudouridine or N1-methyl-pseudouridine, optionally N1-methyl-pseudouridine.

501. The composition of any one of claims 496-500, wherein the mRNA encoding the Cas protein comprises a 5′ cap.

502. The composition of any one of claims 496-501, wherein the mRNA encoding the Cas protein comprises a polyadenylation sequence.

503. The composition of any one of claims 496-502, wherein the mRNA encoding the Cas protein comprises the sequence set forth in SEQ ID NO: 226, 225, or 12.

504. The composition of any one of claims 467-503, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine or N1-methyl-pseudouridine, optionally N1-methyl-pseudouridine, comprises a 5′ cap, and comprises a polyadenylation sequence.

505. The composition of any one of claims 467-504, wherein the guide RNA in the form of RNA, and the guide RNA comprises SEQ ID NO: 68 or 100, and
wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 226, 225, or 12.

506. The composition of claim 505, wherein the nucleic acid construct comprises from 5′ to 3′: a splice acceptor, the lysosomal alpha-glucosidase coding sequence, and a polyadenylation signal or sequence,
wherein the lysosomal alpha-glucosidase coding sequence comprises any one of SEQ ID NOS: 174-182 and 205-212, optionally wherein the lysosomal alpha-glucosidase coding sequence comprises SEQ ID NO: 176,
wherein the nucleic acid construct does not comprise a promoter that drives the expression of the lysosomal alpha-glucosidase,
wherein the nucleic acid construct does not comprise a homology arm, and
wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

507. The composition of any one of claims 467-504, wherein the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and
wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine or N1-methyl-pseudouridine, optionally N1-methyl-pseudouridine, comprises a 5′ cap, and comprises a polyadenylation sequence.

508. The composition of claim 507, wherein the nucleic acid construct comprises from 5′ to 3′: a splice acceptor, the lysosomal alpha-glucosidase coding sequence, and a polyadenylation signal or sequence,
wherein the lysosomal alpha-glucosidase coding sequence comprises any one of SEQ ID NOS: 174-182 and 205-212, optionally wherein the lysosomal alpha-glucosidase coding sequence comprises SEQ ID NO: 176,
wherein the nucleic acid construct does not comprise a promoter that drives the expression of the lysosomal alpha-glucosidase,
wherein the nucleic acid construct does not comprise a homology arm, and
wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

509. The composition of any one of claims 467-508, wherein the Cas protein or the nucleic acid encoding the Cas protein and the guide RNA or the one or more DNAs encoding the guide RNA are associated with a lipid nanoparticle.

510. The composition of claim 509, wherein the lipid nanoparticle comprises a cationic lipid, a neutral lipid, a helper lipid, and a stealth lipid.

511. The composition of claim 510, wherein the cationic lipid is Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate).

512. The composition of claim 510 or 511, wherein the neutral lipid is distearoylphosphatidylcholine or 1,2-distearoyl-sn-glycero-3-phosphocholine (DSPC).

513. The composition of any one of claims 510-512, wherein the helper lipid is cholesterol.

514. The composition of any one of claims 510-513, wherein the stealth lipid is 1,2-dimyristoyl-rac-glycero-3-methoxypolyethylene glycol-2000 (PEG2k-DMG).

515. The composition of any one of claims 510-514, wherein the cationic lipid is Lipid A, the neutral lipid is DSPC, the helper lipid is cholesterol, and the stealth lipid is PEG2k-DMG.

516. The composition of any one of claims 509-515, wherein the lipid nanoparticle comprises four lipids at the following molar ratios: about 50 mol% Lipid A, about 9 mol% DSPC, about 38 mol% cholesterol, and about 3 mol% PEG2k-DMG.

517. The composition of any one of claims 467-516, wherein the albumin gene is a human albumin gene,
wherein the guide RNA in the form of RNA, and the guide RNA comprises SEQ ID NO: 68 or 100,
wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 226, 225, or 12, and
wherein the guide RNA and the mRNA encoding the Cas protein are associated with a lipid nanoparticle comprising Lipid A, DSPC, cholesterol, and PEG2k-DMG, optionally at the following molar ratios: about 50 mol% Lipid A, about 9 mol% DSPC, about 38 mol% cholesterol, and about 3 mol% PEG2k-DMG.

518. The composition of claim 517, wherein the nucleic acid construct comprises from 5′ to 3′: a splice acceptor, the lysosomal alpha-glucosidase coding sequence, and a polyadenylation signal or sequence,
wherein the lysosomal alpha-glucosidase coding sequence comprises any one of SEQ ID NOS: 174-182 and 205-212, optionally wherein the lysosomal alpha-glucosidase coding sequence comprises SEQ ID NO: 176,
wherein the nucleic acid construct does not comprise a promoter that drives the expression of the lysosomal alpha-glucosidase,
wherein the nucleic acid construct does not comprise a homology arm, and
wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

519. The composition of any one of claims 467-516, wherein the albumin gene is a human albumin gene,
wherein the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA,
wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine or N1-methyl-pseudouridine, optionally N1-methyl-pseudouridine, comprises a 5′ cap, and comprises a polyadenylation sequence, and
wherein the guide RNA and the mRNA encoding the Cas protein are associated with a lipid nanoparticle comprising Lipid A, DSPC, cholesterol, and PEG2k-DMG, optionally at the following molar ratios: about 50 mol% Lipid A, about 9 mol% DSPC, about 38 mol% cholesterol, and about 3 mol% PEG2k-DMG.

520. The composition of claim 519, wherein the nucleic acid construct comprises from 5′ to 3′: a splice acceptor, the lysosomal alpha-glucosidase coding sequence, and a polyadenylation signal or sequence,
wherein the lysosomal alpha-glucosidase coding sequence comprises any one of SEQ ID NOS: 174-182 and 205-212, optionally wherein the lysosomal alpha-glucosidase coding sequence comprises SEQ ID NO: 176,
wherein the nucleic acid construct does not comprise a promoter that drives the expression of the lysosomal alpha-glucosidase,
wherein the nucleic acid construct does not comprise a homology arm, and
wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

521. The composition of any one of claims 434-520, for use in a method of inserting the lysosomal alpha-glucosidase coding sequence into a target genomic locus in a cell or a population of cells.

522. The composition of any one of claims 434-520, for use in a method of expressing the lysosomal alpha-glucosidase from a target genomic locus in a cell or a population of cells or for use in a method of expressing the lysosomal alpha-glucosidase in a cell or a population of cells.

523. The composition of any one of claims 434-520, for use in a method of inserting the lysosomal alpha-glucosidase coding sequence into a target genomic locus in a cell or a population of cells in a subject.

524. The composition of any one of claims 434-520, for use in a method of expressing the lysosomal alpha-glucosidase from a target genomic locus in a cell or a population of cells in a subject or for use in a method of expressing the lysosomal alpha-glucosidase in a cell or a population of cells in a subject.

525. The composition of any one of claims 434-520, for use in a method of treating a lysosomal alpha-glucosidase deficiency in a subject in need thereof.

526. The composition of any one of claims 434-520, for use in a method of reducing glycogen accumulation in a tissue in a subject in need thereof.

527. The composition of any one of claims 434-520, for use in a method of treating Pompe disease in a subject in need thereof.

528. The composition of any one of claims 434-520, for use in a method of preventing or reducing the onset of a sign or symptom of Pompe disease in a neonatal subject in need thereof.

529. A cell comprising the composition of any one of claims 434-520.

530. The cell of claim 529, wherein the nucleic acid construct is integrated into a target genomic locus, and wherein the lysosomal alpha-glucosidase is expressed from the target genomic locus, or wherein the nucleic acid construct is integrated into intron 1 of an endogenous albumin locus, and wherein the lysosomal alpha-glucosidase is expressed from the endogenous albumin locus.

531. The cell of claim 529 or 530, wherein the cell is a liver cell.

532. The cell of claim 531, wherein the liver cell is a hepatocyte.

533. The cell of any one of claims 529-532, wherein the cell is a human cell.

534. The cell of any one of claims 529-533, wherein the cell is a neonatal cell.

535. The cell of claim 534, wherein the neonatal cell is from a human neonatal subject within 24 weeks after birth.

536. The cell of claim 534, wherein the neonatal cell is from a human neonatal subject within 12 weeks after birth.

537. The cell of claim 534, wherein the neonatal cell is from a human neonatal subject within 8 weeks after birth.

538. The cell of claim 534, wherein the neonatal cell is from a human neonatal subject within 4 weeks after birth.

539. The cell of any one of claims 529-533, wherein the cell is not a neonatal cell.

540. The cell of any one of claims 529-539, wherein the cell is in vivo.

541. The cell of any one of claims 529-539, wherein the cell is in vitro or ex vivo.

542. A method of inserting a lysosomal alpha-glucosidase coding sequence into a target genomic locus in a cell or a population of cells, comprising administering to the cell or the population of cells the composition of any one of claims 463-520,
wherein the nuclease agent cleaves the nuclease target site in the target genomic locus, and the nucleic acid construct is inserted into the target genomic locus.

543. A method of expressing a lysosomal alpha-glucosidase in a cell or a population of cells, comprising administering to the cell or the population of cells the composition of any one of claims 434-462,
wherein the lysosomal alpha-glucosidase coding sequence is operably linked to a promoter in the nucleic acid construct and is expressed in the cell or population of cells.

544. A method of expressing a lysosomal alpha-glucosidase from a target genomic locus in a cell or a population of cells, comprising administering to the cell or the population of cells the composition of any one of claims 463-520,
wherein the nuclease agent cleaves the nuclease target site in the target genomic locus, the nucleic acid construct is inserted into the target genomic locus to create a modified target genomic locus, and the lysosomal alpha-glucosidase is expressed from the modified target genomic locus.

545. The method of any one of claims 542-544, wherein the cell is a liver cell or the population of cells is a population of liver cells, optionally wherein the cell is a hepatocyte or the population of cells is a population of hepatocytes.

546. The method of any one of claims 542-545, wherein the cell is a human cell or the population of cells is a population of human cells.

547. The method of any one of claims 542-546, wherein the cell is a neonatal cell or the population of cells is a population of neonatal cells.

548. The method of claim 547, wherein the neonatal cell or the population of neonatal cells is from a human neonatal subject within 24 weeks after birth.

549. The method of claim 547, wherein the neonatal cell or the population of neonatal cells is from a human neonatal subject within 12 weeks after birth.

550. The method of claim 547, wherein the neonatal cell or the population of neonatal cells is from a human neonatal subject within 8 weeks after birth.

551. The method of claim 547, wherein the neonatal cell or the population of neonatal cells is from a human neonatal subject within 4 weeks after birth.

552. The method of any one of claims 542-546, wherein the cell is not a neonatal cell or the population of cells is not a population of neonatal cells.

553. The method of any one of claims 542-552, wherein the cell is in vitro or ex vivo or the population of cells is in vitro or ex vivo.

554. The method of any one of claims 542-552, wherein the cell is in vivo in a subject or the population of cells is in vivo in a subject.

555. A method of inserting a lysosomal alpha-glucosidase coding sequence into a target genomic locus in a cell in a subject, comprising administering to the subject the composition of any one of claims 463-520,
wherein the nuclease agent cleaves the nuclease target site in the target genomic locus, and the nucleic acid construct is inserted into the target genomic locus.

556. A method of expressing a lysosomal alpha-glucosidase in a cell in a subject, comprising administering to the subject the composition of any one of claims 434-462,
wherein the lysosomal alpha-glucosidase coding sequence is operably linked to a promoter in the nucleic acid construct and is expressed in the cell.

557. A method of expressing a lysosomal alpha-glucosidase from a target genomic locus in a cell in a subject, comprising administering to the subject the composition of any one of claims 463-520,
wherein the nuclease agent cleaves the nuclease target site in the target genomic locus, the nucleic acid construct is inserted into the target genomic locus to create a modified target genomic locus, and the lysosomal alpha-glucosidase is expressed from the modified target genomic locus.

558. The method of any one of claims 555-557, wherein the cell is a liver cell, optionally wherein the cell is a hepatocyte.

559. The method of any one of claims 555-558, wherein the cell is a human cell.

560. The method of any one of claims 555-559, wherein the cell is a neonatal cell in a neonatal subject.

561. The method of claim 560, wherein the neonatal subject is a human subject within 24 weeks after birth.

562. The method of claim 560, wherein the neonatal subject is a human subject within 12 weeks after birth.

563. The method of claim 560, wherein the neonatal subject is a human subject within 8 weeks after birth.

564. The method of claim 560, wherein the neonatal subject is a human subject within 4 weeks after birth.

565. The method of any one of claims 555-559, wherein the cell is not a neonatal cell.

566. A method of treating a lysosomal alpha-glucosidase deficiency in a subject in need thereof, comprising administering to the subject the composition of any one of claims 434-462,
wherein the lysosomal alpha-glucosidase coding sequence is operably linked to a promoter in the nucleic acid construct and is expressed in the subject.

567. A method of treating a lysosomal alpha-glucosidase deficiency in a subject in need thereof, comprising administering to the subject the composition of any one of claims 463-520,
wherein the nuclease agent cleaves the nuclease target site in the target genomic locus, the nucleic acid construct is inserted into the target genomic locus to create a modified target genomic locus, and the lysosomal alpha-glucosidase is expressed from the modified target genomic locus.

568. A method of reducing glycogen accumulation in a tissue in a subject in need thereof, comprising administering to the subject the composition of any one of claims 434-462,
wherein the lysosomal alpha-glucosidase coding sequence is operably linked to a promoter in the nucleic acid construct and is expressed in the subject and reduces glycogen accumulation in the tissue.

569. A method of reducing glycogen accumulation in a tissue in a subject in need thereof, comprising administering to the subject the composition of any one of claims 463-520,
wherein the nuclease agent cleaves the nuclease target site in the target genomic locus, the nucleic acid construct is inserted into the target genomic locus to create a modified target genomic locus, and the lysosomal alpha-glucosidase is expressed from the modified target genomic locus and reduces glycogen accumulation in the tissue.

570. The method of any one of claims 554-569, wherein the subject has Pompe disease.

571. A method of treating Pompe disease in a subject in need thereof, comprising administering to the subject the composition of any one of claims 434-462,
wherein the lysosomal alpha-glucosidase coding sequence is operably linked to a promoter in the nucleic acid construct and is expressed in the subject, thereby treating the Pompe disease.

572. A method of treating Pompe disease in a subject in need thereof, comprising administering to the subject the composition of any one of claims 463-520,
wherein the nuclease agent cleaves the nuclease target site in the target genomic locus, the nucleic acid construct is inserted into the target genomic locus to create a modified target genomic locus, and the lysosomal alpha-glucosidase is expressed from the modified target genomic locus, thereby treating the Pompe disease.

573. The method of any one of claims 570-572, wherein the Pompe disease is infantile-onset Pompe disease.

574. The method of any one of claims 570-572, wherein the Pompe disease is late-onset Pompe disease.

575. The method of any one of claims 554-574, wherein the subject is a human subject.

576. The method of any one of claims 554-575, wherein the subject is a neonatal subject.

577. The method of claim 576, wherein the neonatal subject is a human subject within 24 weeks after birth, optionally wherein the neonatal subject is a human subject within 12 weeks after birth, within 8 weeks after birth, or within 4 weeks after birth.

578. The method of any one of claims 554-575, wherein the subject is not a neonatal subject.

579. The method of any one of claims 554-578, wherein the method results in a therapeutically effective level of circulating lysosomal alpha-glucosidase in the subject.

580. The method of any one of claims 554-579, wherein the method reduces glycogen accumulation in skeletal muscle, heart tissue, or central nervous system tissue in the subject.

581. The method of claim 580, wherein the method reduces glycogen accumulation in skeletal muscle and heart tissue in the subject.

582. The method of claim 581, wherein the method results in reduced glycogen levels in skeletal muscle and heart tissue in the subject comparable to wild type levels at the same age.

583. The method of any one of claims 554-582, wherein the method improves muscle strength in the subject or prevents loss of muscle strength in the subject compared to a control subject.

584. The method of claim 583, wherein the method results in the subject having muscle strength comparable to wild type levels at the same age.

585. A method of preventing or reducing the onset of a sign or symptom of Pompe disease in a subject in need thereof, comprising administering to the subject the composition of any one of claims 434-462,
wherein the lysosomal alpha-glucosidase coding sequence is operably linked to a promoter in the nucleic acid construct and is expressed in the subject, thereby preventing or reducing the onset of a sign or symptom of the Pompe disease in the subject.

586. A method of preventing or reducing the onset of a sign or symptom of Pompe disease in a subject in need thereof, comprising administering to the subject the composition of any one of claims 463-520,
wherein the nuclease agent cleaves the nuclease target site, the nucleic acid construct is inserted into the target genomic locus to create a modified target genomic locus, and the lysosomal alpha-glucosidase is expressed from the modified target genomic locus, thereby preventing or reducing the onset of a sign or symptom of the Pompe disease in the subject.

587. The method of claim 585 or 586, wherein the Pompe disease is infantile-onset Pompe disease.

588. The method of claim 585 or 586, wherein the Pompe disease is late-onset Pompe disease.

589. The method of any one of claims 585-588, wherein the method results in a therapeutically effective level of circulating lysosomal alpha-glucosidase in the subject.

590. The method of any one of claims 585-589, wherein the method prevents or reduces glycogen accumulation in skeletal muscle, heart, or central nervous system tissue in the subject.

591. The method of any one of claims 585-590, wherein the method prevents or reduces glycogen accumulation in skeletal muscle and heart tissue in the subject.

592. The method of any one of claims 585-591, wherein the subject is a human subject.

593. The method of any one of claims 585-592, wherein the subject is a neonatal subject.

594. The method of claim 593, wherein the neonatal subject is a human subject within 24 weeks after birth, optionally wherein the neonatal subject is a human subject within 12 weeks after birth.

595. The method of claim 593, wherein the neonatal subject is a human subject within 8 weeks after birth.

596. The method of claim 593, wherein the neonatal subject is a human subject within 4 weeks after birth.

597. The method of any one of claims 585-592, wherein the subject is not a neonatal subject.

598. The method of any one of claims 554-597, wherein the method results in increased expression of lysosomal alpha-glucosidase in the subject compared to a method comprising administering an episomal expression vector encoding the lysosomal alpha-glucosidase to a control subject.

599. The method of any one of claims 554-598, wherein the method results in increased serum levels of the lysosomal alpha-glucosidase in the subject compared to a method comprising administering an episomal expression vector encoding the lysosomal alpha-glucosidase to a control subject.

600. The method of any one of claims 554-599, wherein the method results in serum levels of the lysosomal alpha-glucosidase in the subject of at least about 1 µg/mL, at least about 2 µg/mL, at least about 3 µg/mL, at least about 4 µg/mL, at least about 5 µg/mL, at least about 6 µg/mL, at least about 7 µg/mL, at least about 8 µg/mL, at least about 9 µg/mL, or at least about 10 µg/mL.

601. The method of any one of claims 554-600, wherein the method results in serum levels of the lysosomal alpha-glucosidase in the subject of at least about 2 µg/mL or at least about 5 µg/mL.

602. The method of any one of claims 554-601, wherein the method results in serum levels of the lysosomal alpha-glucosidase in the subject of between about 2 µg/mL and about 30 µg/mL or between about 2 µg/mL and about 20 µg/mL.

603. The method of any one of claims 554-602, wherein the method results in serum levels of the lysosomal alpha-glucosidase in the subject of between about 5 µg/mL and about 30 µg/mL or between about 5 µg/mL and about 20 µg/mL.

604. The method of any one of claims 554-603, wherein the method achieves lysosomal alpha-glucosidase activity levels of at least about 40% of normal, at least about 45% of normal, at least about 50%, at least about 60%, at least about 70%, at least about 80%, at least about 90%, or 100% of normal.

605. The method of any one of claims 554-604, wherein:
(I) the subject has infantile-onset Pompe disease, and the method achieves lysosomal alpha-glucosidase expression or activity levels of at least about 1% or more than about 1% of normal; or
(II) the subject has late-onset Pompe disease, and the method achieves lysosomal alpha-glucosidase expression or activity levels of at least about 40% of normal or more than about 40% of normal.

606. The method of any one of claims 554-605, wherein the expression or activity of the lysosomal alpha-glucosidase is at least 50% of the expression or activity of the lysosomal alpha-glucosidase at a peak level of expression measured for the subject at 24 weeks after the administering.

607. The method of any one of claims 554-606, wherein the expression or activity of the lysosomal alpha-glucosidase is at least 50% of the expression or activity of the lysosomal alpha-glucosidase at a peak level of expression measured for the subject at one year after the administering.

608. The method of any one of claims 554-607, wherein the expression or activity of the lysosomal alpha-glucosidase is at least 60% of the expression or activity of the lysosomal alpha-glucosidase at a peak level of expression measured for the subject at 24 weeks after the administering.

609. The method of any one of claims 554-608, wherein the expression or activity of the lysosomal alpha-glucosidase is at least 50% of the expression or activity of the lysosomal alpha-glucosidase at a peak level of expression measured for the subject at two years after the administering.

610. The method of any one of claims 554-609, wherein the expression or activity of the lysosomal alpha-glucosidase is at least 60% of the expression or activity of the lysosomal alpha-glucosidase at a peak level of expression measured for the subject at 2 years after the administering.

611. The method of any one of claims 554-610, wherein the expression or activity of the lysosomal alpha-glucosidase is at least 60% of the expression or activity of the lysosomal alpha-glucosidase at a peak level of expression measured for the subject at 24 weeks after the administering.

612. The method of any one of claim 554-611, wherein the method further comprises assessing preexisting AAV immunity in the subject prior to administering the nucleic acid construct to the subject.

613. The method of claim 612, wherein the preexisting AAV immunity is preexisting AAV8 immunity.

614. The method of claim 612 or 613, wherein assessing preexisting AAV immunity comprises assessing immunogenicity using a total antibody immune assay or a neutralizing antibody assay.

615. The method of any one of claims 542-614, wherein the nucleic acid construct is administered simultaneously with the nuclease agent or the one or more nucleic acids encoding the nuclease agent.

616. The method of any one of claims 542-614, wherein the nucleic acid construct is not administered simultaneously with the nuclease agent or the one or more nucleic acids encoding the nuclease agent.

617. The method of claim 616, wherein the nucleic acid construct is administered prior to the nuclease agent or the one or more nucleic acids encoding the nuclease agent.

618. The method of claim 616, wherein the nucleic acid construct is administered after the nuclease agent or the one or more nucleic acids encoding the nuclease agent.

619. A method of inserting a nucleic acid encoding a multidomain therapeutic protein comprising a delivery domain fused to a lysosomal alpha-glucosidase into a target genomic locus in a neonatal cell or a population of neonatal cells, comprising administering to the neonatal cell or the population of neonatal cells:
(a) a nucleic acid construct comprising a coding sequence for the multidomain therapeutic protein comprising the delivery domain fused to the lysosomal alpha-glucosidase, optionally wherein the delivery domain is a CD63-binding delivery domain or a TfR-binding delivery domain; and
(b) a nuclease agent or one or more nucleic acids encoding the nuclease agent, wherein the nuclease agent targets a nuclease target site in the target genomic locus,
wherein the nuclease agent cleaves the nuclease target site, and the nucleic acid construct is inserted into the target genomic locus.

620. A method of inserting a nucleic acid encoding a multidomain therapeutic protein comprising a CD63-binding delivery domain fused to a lysosomal alpha-glucosidase into a target genomic locus in a neonatal cell or a population of neonatal cells, comprising administering to the neonatal cell or the population of neonatal cells:
(a) a nucleic acid construct comprising a coding sequence for the multidomain therapeutic protein comprising the CD63-binding delivery domain fused to the lysosomal alpha-glucosidase; and
(b) a nuclease agent or one or more nucleic acids encoding the nuclease agent, wherein the nuclease agent targets a nuclease target site in the target genomic locus,
wherein the nuclease agent cleaves the nuclease target site, and the nucleic acid construct is inserted into the target genomic locus.

621. A method of expressing a multidomain therapeutic protein comprising a delivery domain fused to a lysosomal alpha-glucosidase from a target genomic locus in a neonatal cell or a population of neonatal cells, comprising administering to the neonatal cell or the population of neonatal cells:
(a) a nucleic acid construct comprising a coding sequence for the multidomain therapeutic protein comprising the delivery domain fused to the lysosomal alpha-glucosidase, optionally wherein the delivery domain is a CD63-binding delivery domain or a TfR-binding delivery domain; and
(b) a nuclease agent or one or more nucleic acids encoding the nuclease agent, wherein the nuclease agent targets a nuclease target site in the target genomic locus,
wherein the nuclease agent cleaves the nuclease target site, the nucleic acid construct is inserted into the target genomic locus to create a modified target genomic locus, and the multidomain therapeutic protein comprising the delivery domain fused to the lysosomal alpha-glucosidase is expressed from the modified target genomic locus.

622. A method of expressing a multidomain therapeutic protein comprising a CD63-binding delivery domain fused to a lysosomal alpha-glucosidase from a target genomic locus in a neonatal cell or a population of neonatal cells, comprising administering to the neonatal cell or the population of neonatal cells:
(a) a nucleic acid construct comprising a coding sequence for the multidomain therapeutic protein comprising the CD63-binding delivery domain fused to the lysosomal alpha-glucosidase; and
(b) a nuclease agent or one or more nucleic acids encoding the nuclease agent, wherein the nuclease agent targets a nuclease target site in the target genomic locus,
wherein the nuclease agent cleaves the nuclease target site, the nucleic acid construct is inserted into the target genomic locus to create a modified target genomic locus, and the multidomain therapeutic protein comprising the CD63-binding delivery domain fused to the lysosomal alpha-glucosidase is expressed from the modified target genomic locus.

623. A method of inserting a nucleic acid encoding a multidomain therapeutic protein comprising a delivery domain fused to a lysosomal alpha-glucosidase into a target genomic locus in a neonatal cell in a neonatal subject, comprising administering to the neonatal subject:
(a) a nucleic acid construct comprising a coding sequence for the multidomain therapeutic protein comprising the delivery domain fused to the lysosomal alpha-glucosidase, optionally wherein the delivery domain is a CD63-binding delivery domain or a TfR-binding delivery domain; and
(b) a nuclease agent or one or more nucleic acids encoding the nuclease agent, wherein the nuclease agent targets a nuclease target site in the target genomic locus,
wherein the nuclease agent cleaves the nuclease target site, and the nucleic acid construct is inserted into the target genomic locus.

624. A method of inserting a nucleic acid encoding a multidomain therapeutic protein comprising a CD63-binding delivery domain fused to a lysosomal alpha-glucosidase into a target genomic locus in a neonatal cell in a neonatal subject, comprising administering to the neonatal subject:
(a) a nucleic acid construct comprising a coding sequence for the multidomain therapeutic protein comprising the CD63-binding delivery domain fused to the lysosomal alpha-glucosidase; and
(b) a nuclease agent or one or more nucleic acids encoding the nuclease agent, wherein the nuclease agent targets a nuclease target site in the target genomic locus,
wherein the nuclease agent cleaves the nuclease target site, and the nucleic acid construct is inserted into the target genomic locus.

625. A method of expressing a multidomain therapeutic protein comprising a delivery domain fused to a lysosomal alpha-glucosidase protein from a target genomic locus in a neonatal cell in a neonatal subject, comprising administering to the neonatal subject:
(a) a nucleic acid construct comprising a coding sequence for the multidomain therapeutic protein comprising the delivery domain fused to the lysosomal alpha-glucosidase, optionally wherein the delivery domain is a CD63-binding delivery domain or a TfR-binding delivery domain; and
(b) a nuclease agent or one or more nucleic acids encoding the nuclease agent, wherein the nuclease agent targets a nuclease target site in the target genomic locus,
wherein the nuclease agent cleaves the nuclease target site, the nucleic acid construct is inserted into the target genomic locus to create a modified target genomic locus, and the multidomain therapeutic protein comprising the delivery domain fused to the lysosomal alpha-glucosidase is expressed from the modified target genomic locus.

626. A method of expressing a multidomain therapeutic protein comprising a CD63-binding delivery domain fused to a lysosomal alpha-glucosidase protein from a target genomic locus in a neonatal cell in a neonatal subject, comprising administering to the neonatal subject:
(a) a nucleic acid construct comprising a coding sequence for the multidomain therapeutic protein comprising the CD63-binding delivery domain fused to the lysosomal alpha-glucosidase; and
(b) a nuclease agent or one or more nucleic acids encoding the nuclease agent, wherein the nuclease agent targets a nuclease target site in the target genomic locus,
wherein the nuclease agent cleaves the nuclease target site, the nucleic acid construct is inserted into the target genomic locus to create a modified target genomic locus, and the multidomain therapeutic protein comprising the CD63-binding delivery domain fused to the lysosomal alpha-glucosidase is expressed from the modified target genomic locus.

627. A method of treating a lysosomal alpha-glucosidase deficiency in a neonatal subject in need thereof, comprising administering to the neonatal subject:
(a) a nucleic acid construct comprising a coding sequence for a multidomain therapeutic protein comprising a delivery domain fused to a lysosomal alpha-glucosidase, optionally wherein the delivery domain is a CD63-binding delivery domain or a TfR-binding delivery domain; and
(b) a nuclease agent or one or more nucleic acids encoding the nuclease agent, wherein the nuclease agent targets a nuclease target site in a target genomic locus,
wherein the nuclease agent cleaves the nuclease target site, the nucleic acid construct is inserted into the target genomic locus to create a modified target genomic locus, and the multidomain therapeutic protein comprising the delivery domain fused to the lysosomal alpha-glucosidase is expressed from the modified target genomic locus.

628. A method of treating a lysosomal alpha-glucosidase deficiency in a neonatal subject in need thereof, comprising administering to the neonatal subject:
(a) a nucleic acid construct comprising a coding sequence for a multidomain therapeutic protein comprising a CD63-binding delivery domain fused to a lysosomal alpha-glucosidase; and
(b) a nuclease agent or one or more nucleic acids encoding the nuclease agent, wherein the nuclease agent targets a nuclease target site in a target genomic locus,
wherein the nuclease agent cleaves the nuclease target site, the nucleic acid construct is inserted into the target genomic locus to create a modified target genomic locus, and the multidomain therapeutic protein comprising the CD63-binding delivery domain fused to the lysosomal alpha-glucosidase is expressed from the modified target genomic locus.

629. A method of reducing glycogen accumulation in a tissue in a neonatal subject in need thereof, comprising administering to the neonatal subject:
(a) a nucleic acid construct comprising a coding sequence for a multidomain therapeutic protein comprising a delivery domain fused a lysosomal alpha-glucosidase, optionally wherein the delivery domain is a CD63-binding delivery domain or a TfR-binding delivery domain; and
(b) a nuclease agent or one or more nucleic acids encoding the nuclease agent, wherein the nuclease agent targets a nuclease target site in a target genomic locus,
wherein the nuclease agent cleaves the nuclease target site, the nucleic acid construct is inserted into the target genomic locus to create a modified target genomic locus, and the multidomain therapeutic protein comprising the delivery domain fused to the lysosomal alpha-glucosidase is expressed from the modified target genomic locus and reduces glycogen accumulation in the tissue.

630. A method of reducing glycogen accumulation in a tissue in a neonatal subject in need thereof, comprising administering to the neonatal subject:
(a) a nucleic acid construct comprising a coding sequence for a multidomain therapeutic protein comprising a CD63-binding delivery domain fused a lysosomal alpha-glucosidase; and
(b) a nuclease agent or one or more nucleic acids encoding the nuclease agent, wherein the nuclease agent targets a nuclease target site in a target genomic locus,
wherein the nuclease agent cleaves the nuclease target site, the nucleic acid construct is inserted into the target genomic locus to create a modified target genomic locus, and the multidomain therapeutic protein comprising the CD63-binding delivery domain fused to the lysosomal alpha-glucosidase is expressed from the modified target genomic locus and reduces glycogen accumulation in the tissue.

631. A method of treating Pompe disease in a neonatal subject in need thereof, comprising administering to the neonatal subject:
(a) a nucleic acid construct comprising a coding sequence for a multidomain therapeutic protein comprising a delivery domain fused to a lysosomal alpha-glucosidase, optionally wherein the delivery domain is a CD63-binding delivery domain or a TfR-binding delivery domain; and
(b) a nuclease agent or one or more nucleic acids encoding the nuclease agent, wherein the nuclease agent targets a nuclease target site in a target genomic locus,
wherein the nuclease agent cleaves the nuclease target site, the nucleic acid construct is inserted into the target genomic locus to create a modified target genomic locus, and the multidomain therapeutic protein comprising the delivery domain fused to the lysosomal alpha-glucosidase is expressed from the modified target genomic locus, thereby treating the Pompe disease.

632. A method of treating Pompe disease in a neonatal subject in need thereof, comprising administering to the neonatal subject:
(a) a nucleic acid construct comprising a coding sequence for a multidomain therapeutic protein comprising a CD63-binding delivery domain fused to a lysosomal alpha-glucosidase; and
(b) a nuclease agent or one or more nucleic acids encoding the nuclease agent, wherein the nuclease agent targets a nuclease target site in a target genomic locus,
wherein the nuclease agent cleaves the nuclease target site, the nucleic acid construct is inserted into the target genomic locus to create a modified target genomic locus, and the multidomain therapeutic protein comprising the CD63-binding delivery domain fused to the lysosomal alpha-glucosidase is expressed from the modified target genomic locus, thereby treating the Pompe disease.

633. A method of preventing or reducing the onset of a sign or symptom of Pompe disease in a neonatal subject in need thereof, comprising administering to the neonatal subject:
(a) a nucleic acid construct comprising a coding sequence for a multidomain therapeutic protein comprising a delivery domain fused to a lysosomal alpha-glucosidase, optionally wherein the delivery domain is a CD63-binding delivery domain or a TfR-binding delivery domain; and
(b) a nuclease agent or one or more nucleic acids encoding the nuclease agent, wherein the nuclease agent targets a nuclease target site in a target genomic locus,
wherein the nuclease agent cleaves the nuclease target site, the nucleic acid construct is inserted into the target genomic locus to create a modified target genomic locus, and the multidomain therapeutic protein comprising the delivery domain fused to the lysosomal alpha-glucosidase is expressed from the modified target genomic locus, thereby preventing or reducing the onset of a sign or symptom of the Pompe disease in the subject.

634. A method of preventing or reducing the onset of a sign or symptom of Pompe disease in a neonatal subject in need thereof, comprising administering to the neonatal subject:
(a) a nucleic acid construct comprising a coding sequence for a multidomain therapeutic protein comprising a CD63-binding delivery domain fused to a lysosomal alpha-glucosidase; and
(b) a nuclease agent or one or more nucleic acids encoding the nuclease agent, wherein the nuclease agent targets a nuclease target site in a target genomic locus,
wherein the nuclease agent cleaves the nuclease target site, the nucleic acid construct is inserted into the target genomic locus to create a modified target genomic locus, and the multidomain therapeutic protein comprising the CD63-binding delivery domain fused to the lysosomal alpha-glucosidase is expressed from the modified target genomic locus, thereby preventing or reducing the onset of a sign or symptom of the Pompe disease in the subject.

635. A neonatal cell or a population of neonatal cells made by the method of any one of claims 619-634.

636. A neonatal cell or a population of neonatal cells comprising a nucleic acid construct inserted into a target genomic locus, wherein the nucleic acid construct comprises a coding sequence for a multidomain therapeutic protein comprising a delivery domain fused to a lysosomal alpha-glucosidase inserted into a target genomic locus, optionally wherein the delivery domain is a CD63-binding delivery domain or a TfR-binding delivery domain.

637. A neonatal cell or a population of neonatal cells comprising a nucleic acid construct inserted into a target genomic locus, wherein the nucleic acid construct comprises a coding sequence for a multidomain therapeutic protein comprising a CD63-binding delivery domain fused to a lysosomal alpha-glucosidase inserted into a target genomic locus.

638. A method of inserting a nucleic acid encoding a multidomain therapeutic protein comprising a TfR-binding delivery domain fused to a lysosomal alpha-glucosidase into a target genomic locus in a neonatal cell or a population of neonatal cells, comprising administering to the neonatal cell or the population of neonatal cells:
(a) a nucleic acid construct comprising a coding sequence for the multidomain therapeutic protein comprising the TfR-binding delivery domain fused to the lysosomal alpha-glucosidase; and
(b) a nuclease agent or one or more nucleic acids encoding the nuclease agent, wherein the nuclease agent targets a nuclease target site in the target genomic locus,
wherein the nuclease agent cleaves the nuclease target site, and the nucleic acid construct is inserted into the target genomic locus.

639. A method of expressing a multidomain therapeutic protein comprising a TfR-binding delivery domain fused to a lysosomal alpha-glucosidase from a target genomic locus in a neonatal cell or a population of neonatal cells, comprising administering to the neonatal cell or the population of neonatal cells:
(a) a nucleic acid construct comprising a coding sequence for the multidomain therapeutic protein comprising the TfR-binding delivery domain fused to the lysosomal alpha-glucosidase; and
(b) a nuclease agent or one or more nucleic acids encoding the nuclease agent, wherein the nuclease agent targets a nuclease target site in the target genomic locus,
wherein the nuclease agent cleaves the nuclease target site, the nucleic acid construct is inserted into the target genomic locus to create a modified target genomic locus, and the multidomain therapeutic protein comprising the TfR-binding delivery domain fused to the lysosomal alpha-glucosidase is expressed from the modified target genomic locus.

640. A method of inserting a nucleic acid encoding a multidomain therapeutic protein comprising a TfR-binding delivery domain fused to a lysosomal alpha-glucosidase into a target genomic locus in a neonatal cell in a neonatal subject, comprising administering to the neonatal subject:
(a) a nucleic acid construct comprising a coding sequence for the multidomain therapeutic protein comprising the TfR-binding delivery domain fused to the lysosomal alpha-glucosidase; and
(b) a nuclease agent or one or more nucleic acids encoding the nuclease agent, wherein the nuclease agent targets a nuclease target site in the target genomic locus,
wherein the nuclease agent cleaves the nuclease target site, and the nucleic acid construct is inserted into the target genomic locus.

641. A method of expressing a multidomain therapeutic protein comprising a TfR-binding delivery domain fused to a lysosomal alpha-glucosidase protein from a target genomic locus in a neonatal cell in a neonatal subject, comprising administering to the neonatal subject:
(a) a nucleic acid construct comprising a coding sequence for the multidomain therapeutic protein comprising the TfR-binding delivery domain fused to the lysosomal alpha-glucosidase; and
(b) a nuclease agent or one or more nucleic acids encoding the nuclease agent, wherein the nuclease agent targets a nuclease target site in a target gene at the target genomic locus,
wherein the nuclease agent cleaves the nuclease target site, the nucleic acid construct is inserted into the target genomic locus to create a modified target genomic locus, and the multidomain therapeutic protein comprising the TfR-binding delivery domain fused to the lysosomal alpha-glucosidase is expressed from the modified target genomic locus.

642. A method of treating a lysosomal alpha-glucosidase deficiency in a neonatal subject in need thereof, comprising administering to the neonatal subject:
(a) a nucleic acid construct comprising a coding sequence for a multidomain therapeutic protein comprising a TfR-binding delivery domain fused to a lysosomal alpha-glucosidase; and
(b) a nuclease agent or one or more nucleic acids encoding the nuclease agent, wherein the nuclease agent targets a nuclease target site in a target genomic locus,
wherein the nuclease agent cleaves the nuclease target site, the nucleic acid construct is inserted into the target genomic locus to create a modified target genomic locus, and the multidomain therapeutic protein comprising the TfR-binding delivery domain fused to the lysosomal alpha-glucosidase is expressed from the modified target genomic locus.

643. A method of reducing glycogen accumulation in a tissue in a neonatal subject in need thereof, comprising administering to the neonatal subject:
(a) a nucleic acid construct comprising a coding sequence for a multidomain therapeutic protein comprising a TfR-binding delivery domain fused to a lysosomal alpha-glucosidase; and
(b) a nuclease agent or one or more nucleic acids encoding the nuclease agent, wherein the nuclease agent targets a nuclease target site in a target genomic locus,
wherein the nuclease agent cleaves the nuclease target site, the nucleic acid construct is inserted into the target genomic locus to create a modified target genomic locus, and the multidomain therapeutic protein comprising the TfR-binding delivery domain fused to the lysosomal alpha-glucosidase is expressed from the modified target genomic locus and reduces glycogen accumulation in the tissue.

644. A method of treating Pompe disease in a neonatal subject in need thereof, comprising administering to the neonatal subject:
(a) a nucleic acid construct comprising a coding sequence for a multidomain therapeutic protein comprising a TfR-binding delivery domain fused to a lysosomal alpha-glucosidase; and
(b) a nuclease agent or one or more nucleic acids encoding the nuclease agent, wherein the nuclease agent targets a nuclease target site in a target genomic locus,
wherein the nuclease agent cleaves the nuclease target site, the nucleic acid construct is inserted into the target genomic locus to create a modified target genomic locus, and the multidomain therapeutic protein comprising the TfR-binding delivery domain fused to the lysosomal alpha-glucosidase is expressed from the modified target genomic locus, thereby treating the Pompe disease.

645. A method of preventing or reducing the onset of a sign or symptom of Pompe disease in a neonatal subject in need thereof, comprising administering to the neonatal subject:
(a) a nucleic acid construct comprising a coding sequence for a multidomain therapeutic protein comprising a TfR-binding delivery domain fused to a lysosomal alpha-glucosidase; and
(b) a nuclease agent or one or more nucleic acids encoding the nuclease agent, wherein the nuclease agent targets a nuclease target site in a target genomic locus,
wherein the nuclease agent cleaves the nuclease target site, the nucleic acid construct is inserted into the target genomic locus to create a modified target genomic locus, and the multidomain therapeutic protein comprising the TfR-binding delivery domain fused to the lysosomal alpha-glucosidase is expressed from the modified target genomic locus, thereby preventing or reducing the onset of a sign or symptom of the Pompe disease in the subject.

646. A neonatal cell or a population of neonatal cells made by the method of any one of claims 638-645.

647. A neonatal cell or a population of neonatal cells comprising a nucleic acid construct inserted into a target genomic locus, wherein the nucleic acid construct comprises a coding sequence for a multidomain therapeutic protein comprising a TfR-binding delivery domain fused to a lysosomal alpha-glucosidase inserted into a target genomic locus.
